(19) United States
(12) Patent Application Publication
(10) Pub. No.: US 2022/0169628 A1
(43) Pub. Date: Jun. 2, 2022

(54) THERAPEUTIC AGENTS AND METHODS OF TREATMENT

(71) Applicant: University of Florida Research Foundation, Incorporated, Gainesville, FL (US)

(72) Inventors: Guangrong Zheng, Gainesville, FL (US); Daohong Zhou, Waldo, FL (US); Pratik Pal, Gainesville, FL (US); Xingui Liu, Gainesville, FL (US); Dinesh Thummuri, Gainesville, FL (US); Wanyi HU, Gainesville, FL (US); Peiyi Zhang, Gainesville, FL (US); Dongwen Lyu, Gainesville, FL (US); Yaxia Yuan, Gainesville, FL (US); Xuan Zhang, Gainesville, FL (US)

(73) Assignee: University of Florida Research Foundation, Incorporated, Gainesville, FL (US)

(21) Appl. No.: 17/429,207

(22) PCT Filed: Feb. 7, 2020
(86) PCT No.: PCT/US2020/017364

§ 371 Date: Aug. 6, 2021

Related U.S. Application Data
(60) Provisional application No. 62/803,299, filed on Feb. 8, 2019.

(51) Int. Cl. C07D 401/04; C07D 417/12; A61P 35/02; C07D 417/14; A61K 45/06

(52) U.S. Cl. CPC C07D 401/04; C07D 417/12; A61K 45/06; C07D 417/14; A61P 35/02

(57) ABSTRACT

The invention is directed towards compounds (e.g., Formula (I)), their mechanism of action, and methods of modulating proliferation activity, and methods of treating diseases and disorders using the compounds described herein (e.g., Formula (I)).

### CROSS REFERENCE TO RELATED APPLICATIONS

This application claims priority to U.S. Provisional Application No. 62/803,299, filed Feb. 8, 2019, which is incorporated herein by reference in its entirety.

### GOVERNMENT SUPPORT INFORMATION

This invention was made with government support under Grant Nos. CA219836 and CA223371 awarded by the National Institutes of Health. The government has certain rights in the invention.

### BACKGROUND

The B-cell lymphoma 2 (Bcl-2) protein family, consisting of pro- and anti-apoptotic members, plays a critical role in determining cell fate through regulation of the intrinsic apoptosis pathway. The anti-apoptotic Bcl-2 family proteins, such as Bcl-2, Bcl-xL, Bcl-w, and Mcl-1, are upregulated in many cancers and associated with tumor initiation, progression, and resistance to chemo- and targeted therapies. Thus, these anti-apoptotic Bcl-2 proteins are attractive targets for the development of novel anti-cancer agents (Lessene et al., Nat Rev Drug Discov 7: 989-1000, 2008; Vogler et al., Cell Death Differ 2009; 16: 360-367; Delbridge et al., Nat Rev Cancer 16: 99-109, 2016). Numerous BcI-2 small molecule inhibitors have been reported (Bajwa et al., Expert Opin Ther Patents 22:37-55, 2012; Vogler, Adv Med. 1-14, 2014; Ashkenazi et al., 16: 273-284, 2017). The following are some of the BcI-2 small molecule inhibitors that have been investigated at various stages of drug development: ABT-737 (US20070072860), navitoclax (ABT-263, WO2009155386), venetoclax (ABT-199, WO2010138588), obatoclax (GX 15-070, WO2004106328), (−)-gossypol (AT-101, WO2002097053), sabutoclax (BI-97C1, WO2010120943), TW-37 (WO2006023778), BM-1252 (APG-1252), and A-1155463 (VV02010080503).

Venetoclax, a selective Bcl-2 inhibitor, was approved by the FDA in 2016 for the treatment of chronic lymphocytic leukemia (CLL) with 17-p deletion. Venetoclax was designed to have high selectivity for BcI-2 over BcI-xL to avoid the on-target platelet toxicity (Souers et al., Nat Med 19: 202-208, 2013). Platelets depend on Bcl-xL to maintain their viability, therefore dose-limiting thrombocytopenia has been observed in animals and/or humans treated with ABT-737 (Schoenwaelder et al., Blood 118: 1663-1674, 2011), ABT-263 (Tse et al., Cancer Res 68: 3421-3428, 2008; Roberts et al., Bri J Haematol 170: 669-678, 2015), BM-1197 (Bai et al., PLoS ONE 9:e99404, 2014), or A-1155463 (Tao et al., ACS Med Chem Lett 5:1088-1093,2014), due to their inhibition of Bcl-xL. However, many CLL patients are resistant to venetoclax (Roberts et al., N Engl J Med 374: 311-322, 2016) and upregulation of Bcl-xL by microenvironmental survival signals has been identified as the major component accountable for the resistance, consistent with the high efficacy of Bcl-2/Bcl-xL dual inhibitor ABT-263 in killing venetoclax resistant CLL cells (Oppermann et al., Blood 128: 934-947, 2016). In addition, Bcl-xL is generally more frequently overexpressed than Bcl-2 in solid tumors. Importantly, promising results have been documented from preclinical and clinical studies of ABT-263, as a single-agent or in combination with other antitumor agents, against several solid and hematologic malignancies (Delbridge et al., Nat Rev Cancer 16: 99-109, 2016). Therefore, it is highly desirable to develop a strategy that can retain the antitumor versatility and efficacy of the Bcl-xL inhibitors, while spare their on-target platelet toxicity.

Thus, there is a need in the art to develop compounds that can retain the antitumor versatility and efficacy of the Bcl-xL inhibitors, while avoiding their on-target platelet toxicity.

### BRIEF SUMMARY OF THE INVENTION

The invention is directed towards compounds (e.g., Formula (I)), their mechanism of action, and methods of modulating proliferation activity, and methods of treating diseases and disorders using the compounds described herein (e.g., Formula (I)). In another aspect, the disease or disorder is cancer. In another aspect, the cancer is a Bcl-2-mediated cancer. In another aspect, the cancer is chronic lymphocyctic leukemia.

In another aspect, the invention is directed to a compound of Formula (I), or a pharmaceutically acceptable salt thereof:

Y-L₂-R-L₁-Y₂ Formula (I);

wherein L₁ is independently

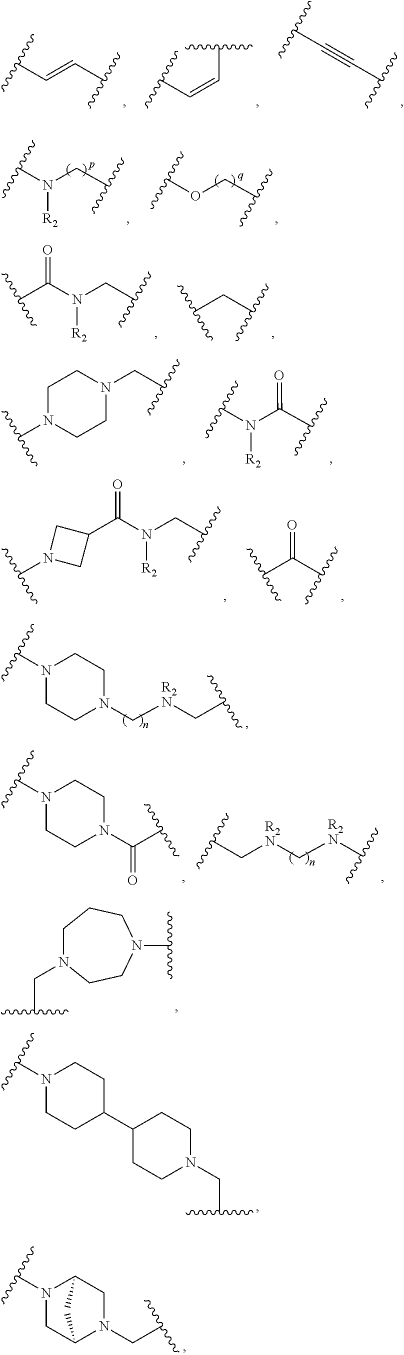

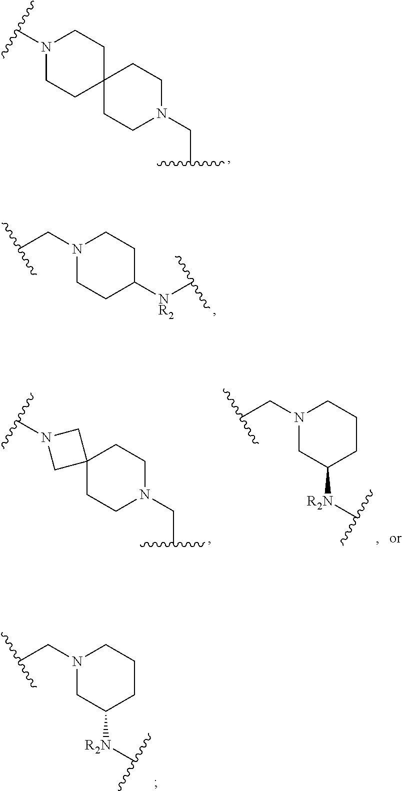

R is independently

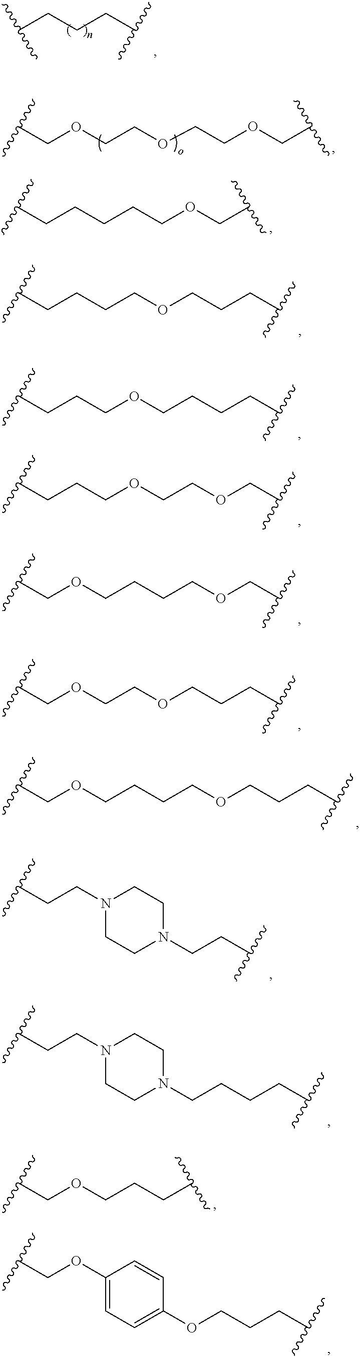

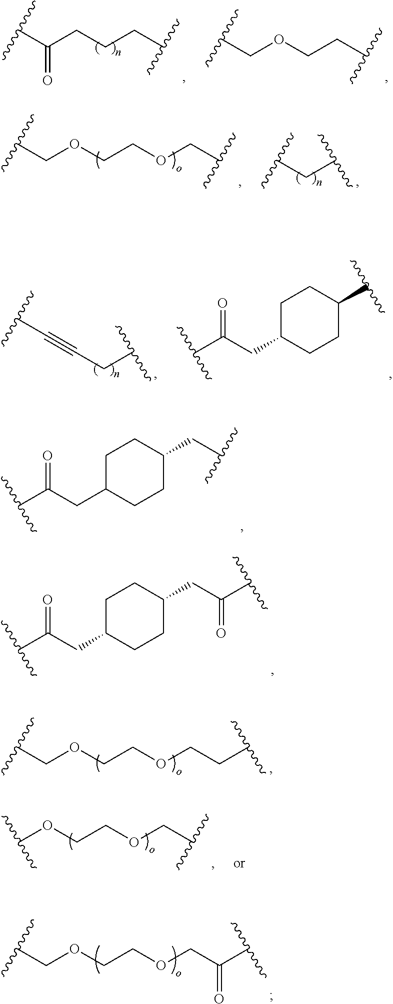

L₂ is independently

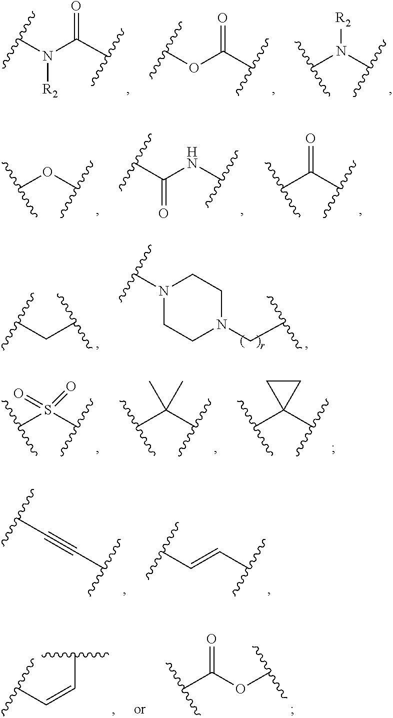

Y is independently

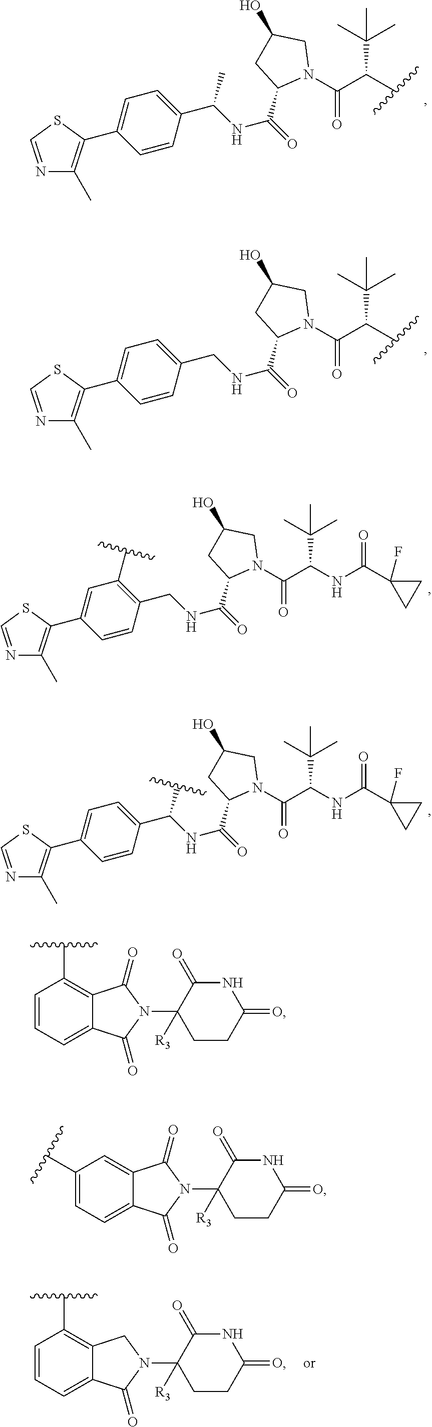

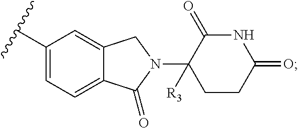

Y₂ is independently

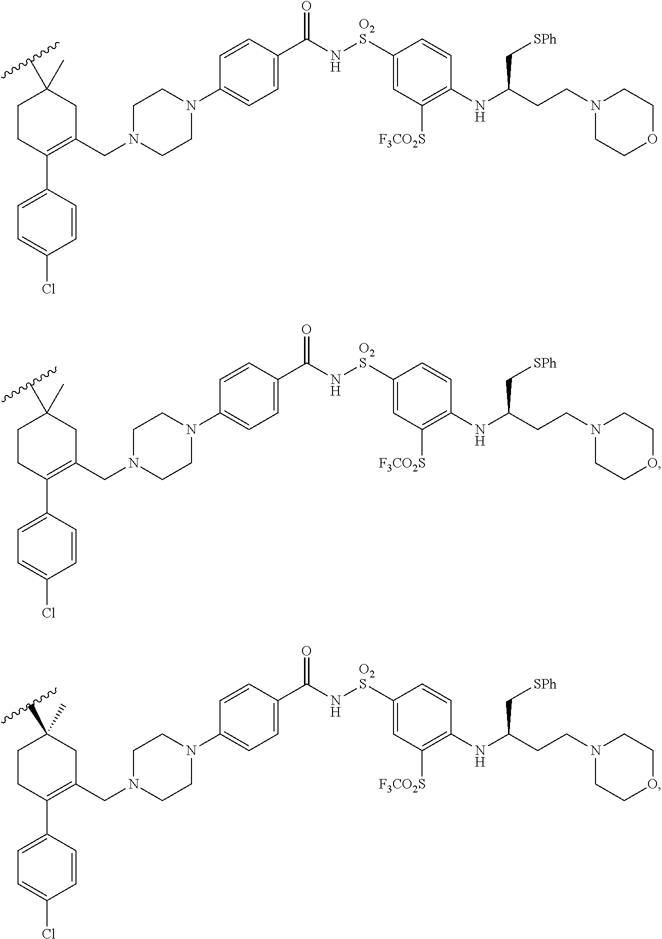

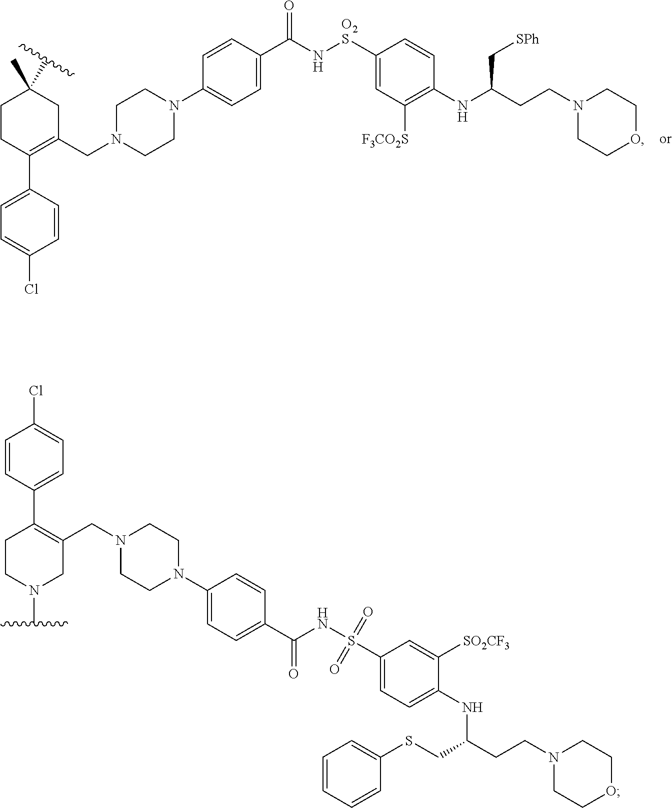

each R₂ is independently H, optionally substituted alkyl, or optionally substituted cycloalkyl;

each R₃ is independently H, D, CH₃, or F; and

each n, o, p, and q is independently 0-10, inclusive.

In another aspect, the invention is a compound of Formula (I), or a pharmaceutically acceptable salt thereof, wherein Y is

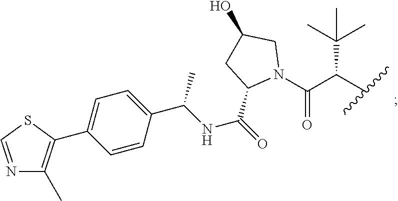 [SMILES: *[C@H](C(=O)N1C[C@H](O)C[C@H]1C(=O)N[C@@H](C)c1ccc(-c2scnc2C)cc1)C(C)(C)C]

### Y₂ is

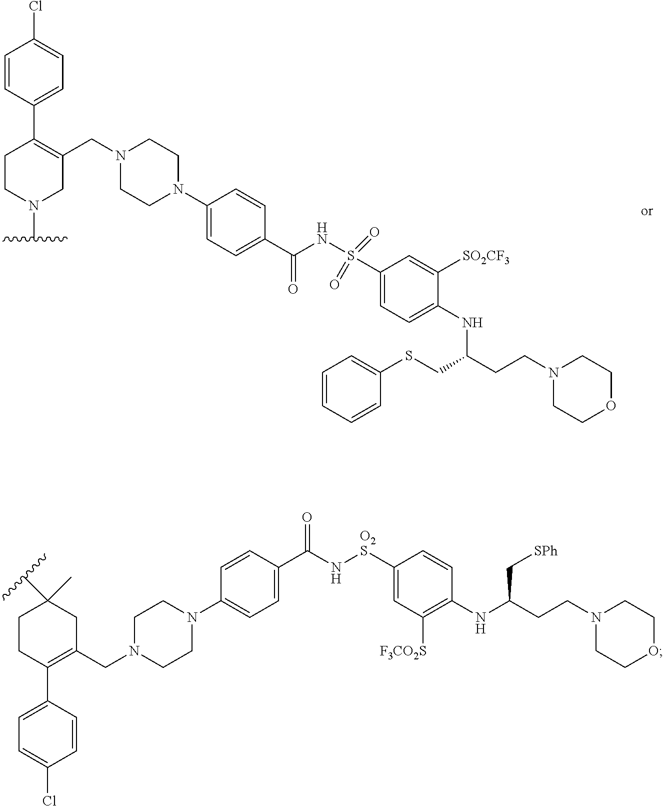

and
R is independently

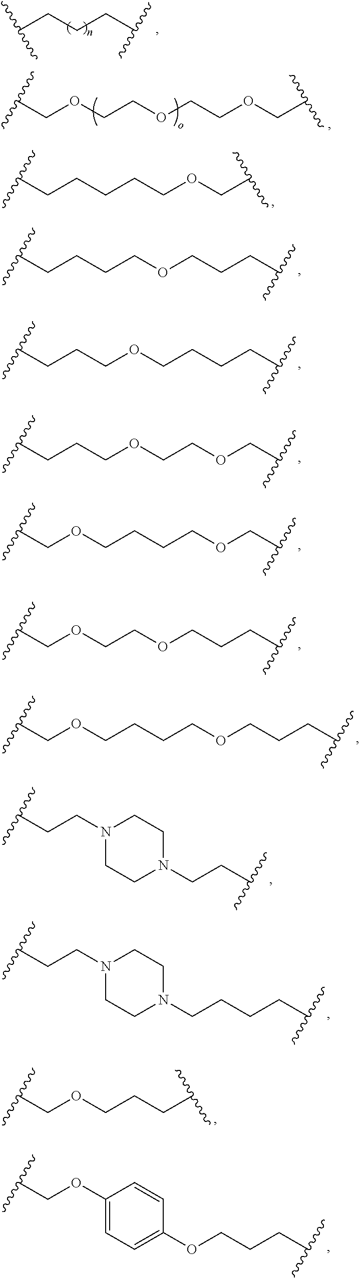

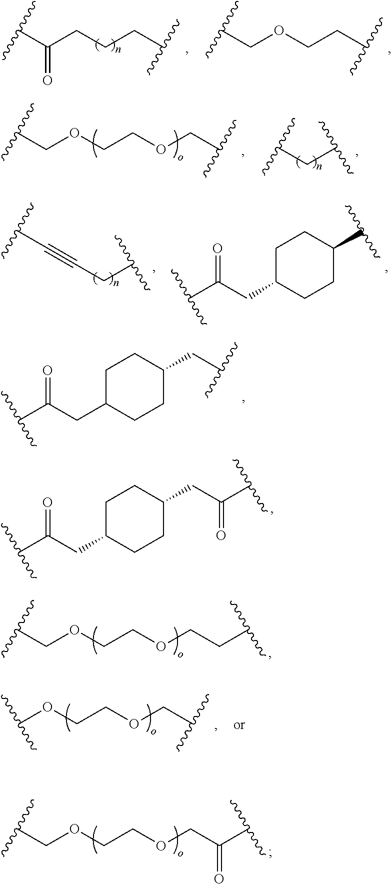

In another aspect, the invention is a compound of Formula (I), or a pharmaceutically acceptable salt thereof, wherein Y is

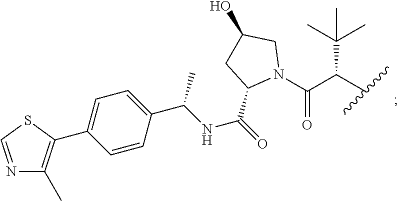 [SMILES: *[C@H](C(=O)N1C[C@H](O)C[C@H]1C(=O)N[C@@H](C)c1ccc(-c2scnc2C)cc1)C(C)(C)C]

### Y₂ is

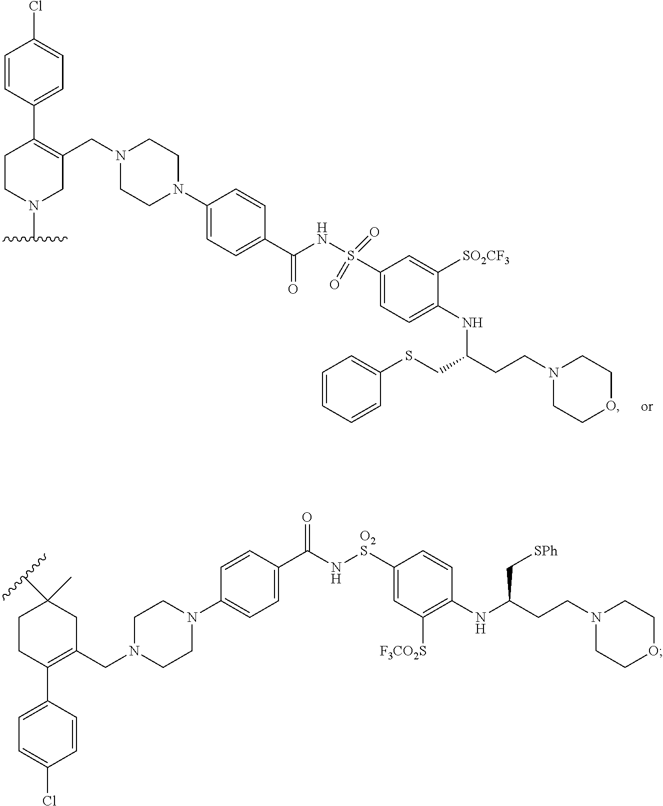

and
L₁ is independently

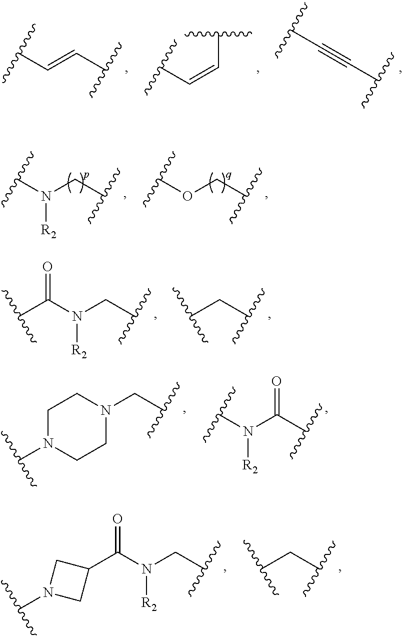

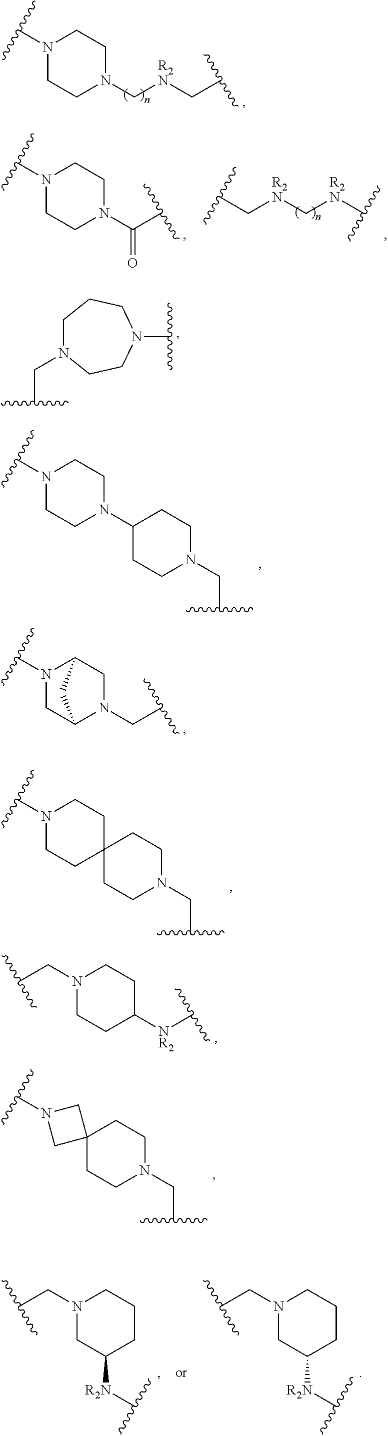

In another aspect, the invention is directed to a compound of Formula (I), or a pharmaceutically acceptable salt, hydrate, solvate, or prodrug thereof:

Y-L₂-R-L₁-Y₂ Formula (I);

wherein L₁ is independently

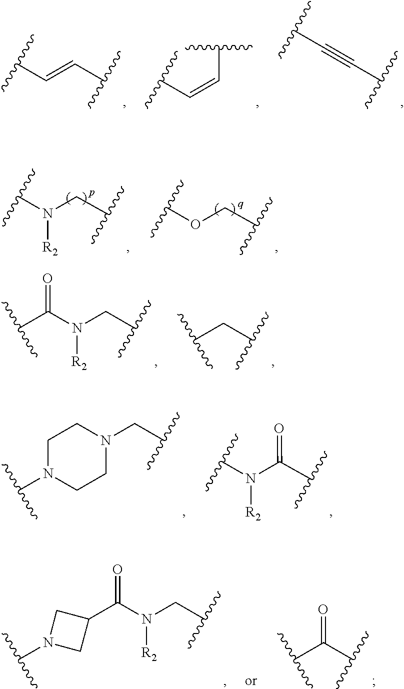

R is independently

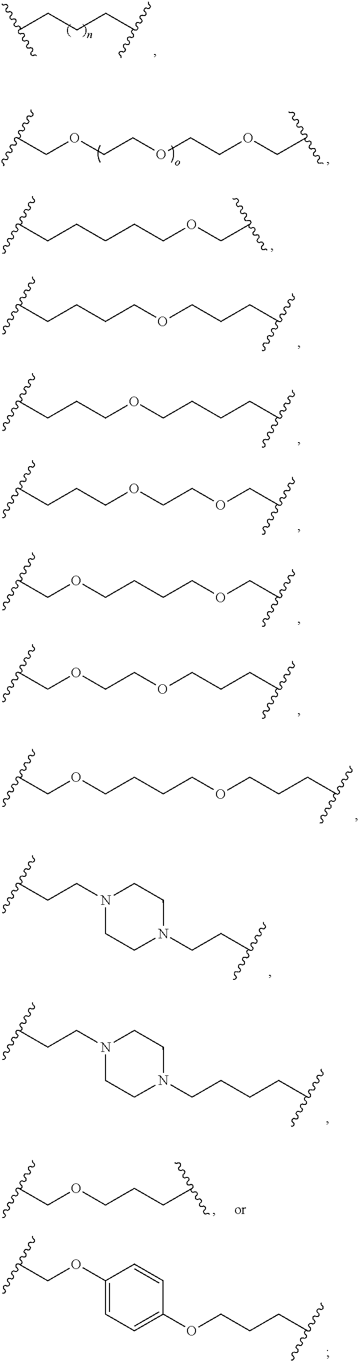

L₂ is independently

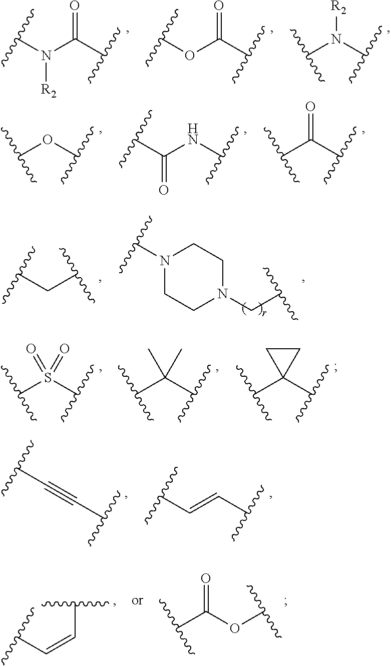

Y is independently

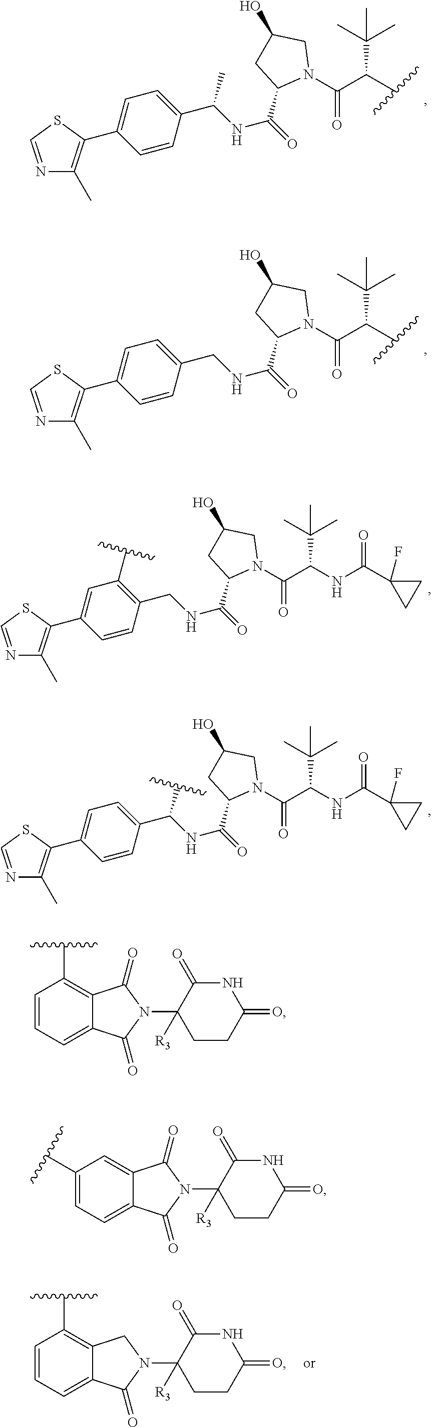

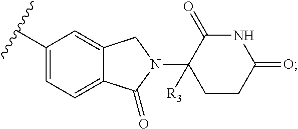

Y₂ is independently

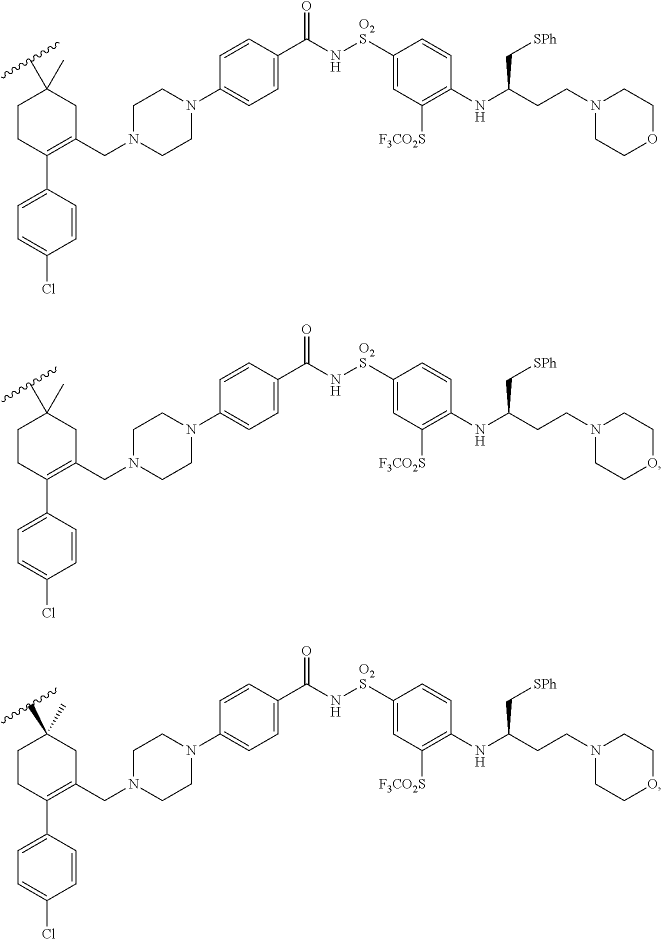

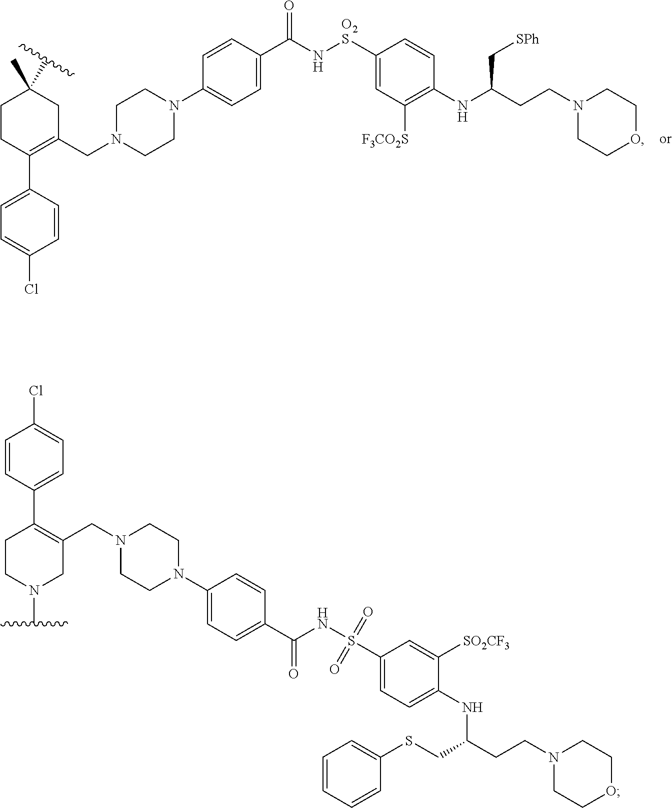

each R₂ is independently H, optionally substituted alkyl, or optionally substituted cycloalkyl;

each R₃ is independently H, D, CH₃, or F; and

each n, o, p, and q is independently 0-10, inclusive.

In another aspect, R is

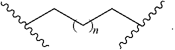

In another aspect, n is 3-8, inclusive.

In another aspect, R is

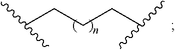

and n is 3-8, inclusive.

In another aspect, L₂ is independently

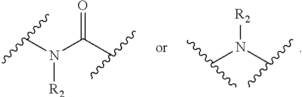

In another aspect, L₂ is independently

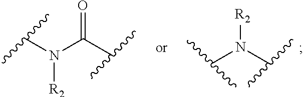

### and R is

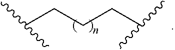

In another aspect, L₂ is independently

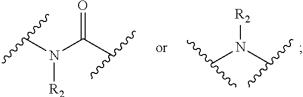

### R is

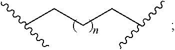

and n is 3-8, inclusive.

In another aspect, L₁ is independently

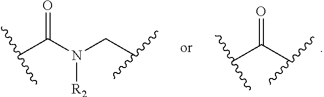

In another aspect, L₁ is independently

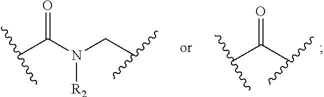

### and R is

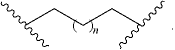

In another aspect, L₁ is independently

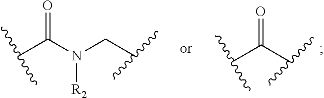

### R is

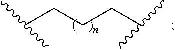

and n is 3-8, inclusive.

In another aspect, L₂ is independently

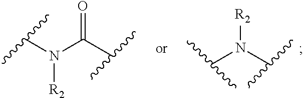

and L₁ is independently

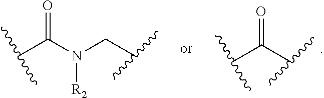

In another aspect, L₂ is independently

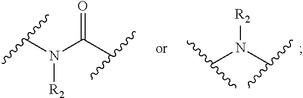

L₁ is independently

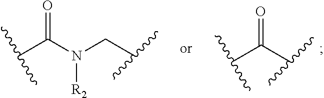

### and R is

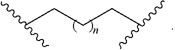

In another aspect, L₂ is independently

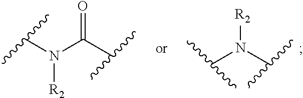

L₁ is independently

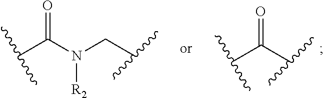

### R is

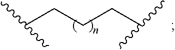

and n is 3-8, inclusive.

In another aspect, Y is

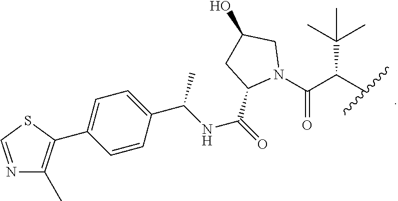 [SMILES: *[C@H](C(=O)N1C[C@H](O)C[C@H]1C(=O)N[C@@H](C)c1ccc(-c2scnc2C)cc1)C(C)(C)C]

In another aspect, Y is

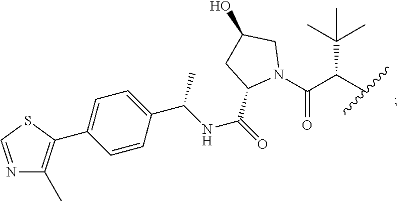 [SMILES: *[C@H](C(=O)N1C[C@H](O)C[C@H]1C(=O)N[C@@H](C)c1ccc(-c2scnc2C)cc1)C(C)(C)C]

### and L₂ is

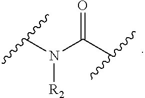

In another aspect, Y is

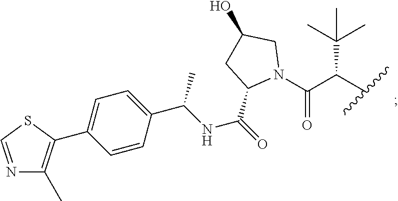 [SMILES: *[C@H](C(=O)N1C[C@H](O)C[C@H]1C(=O)N[C@@H](C)c1ccc(-c2scnc2C)cc1)C(C)(C)C]

### L₂ is

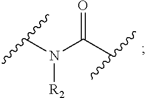

### and R is

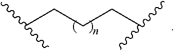

In another aspect, Y is

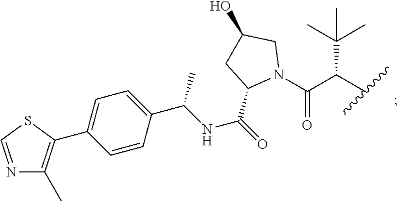 [SMILES: *[C@H](C(=O)N1C[C@H](O)C[C@H]1C(=O)N[C@@H](C)c1ccc(-c2scnc2C)cc1)C(C)(C)C]

### L₂ is

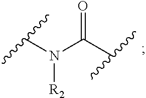

### R is

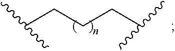

and n is 3-8, inclusive.

In another aspect, Y is

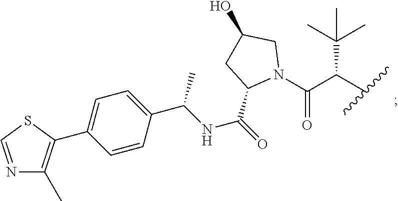 [SMILES: *[C@H](C(=O)N1C[C@H](O)C[C@H]1C(=O)N[C@@H](C)c1ccc(-c2scnc2C)cc1)C(C)(C)C]

### L₂ is

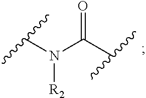

and L₁ is independently

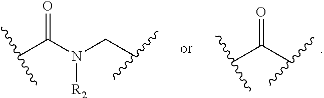

In another aspect, Y is

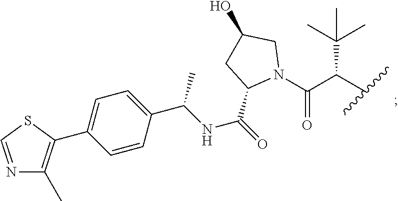 [SMILES: *[C@H](C(=O)N1C[C@H](O)C[C@H]1C(=O)N[C@@H](C)c1ccc(-c2scnc2C)cc1)C(C)(C)C]

### L₂ is

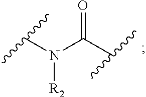

L₁ is independently

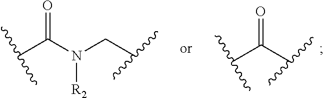

### and R is

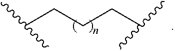

In another aspect Y is

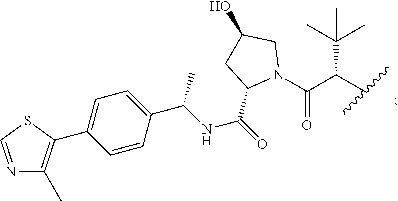 [SMILES: *[C@H](C(=O)N1C[C@H](O)C[C@H]1C(=O)N[C@@H](C)c1ccc(-c2scnc2C)cc1)C(C)(C)C]

### L₂ is

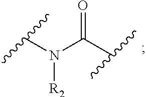

L₁ is independently

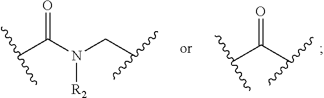

### R is

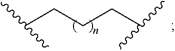

and n is 3-8, inclusive.

In another aspect, Y is

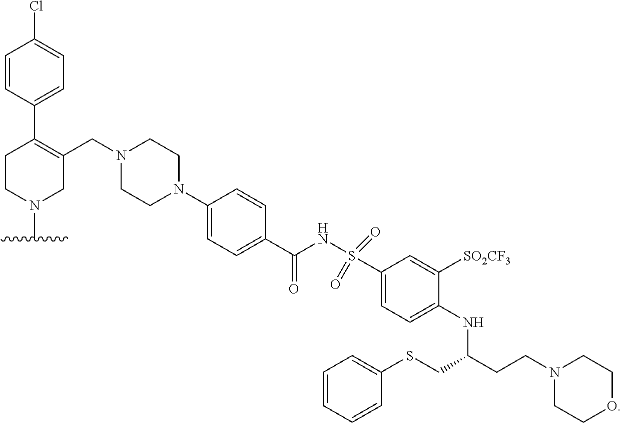

In another aspect, Y is

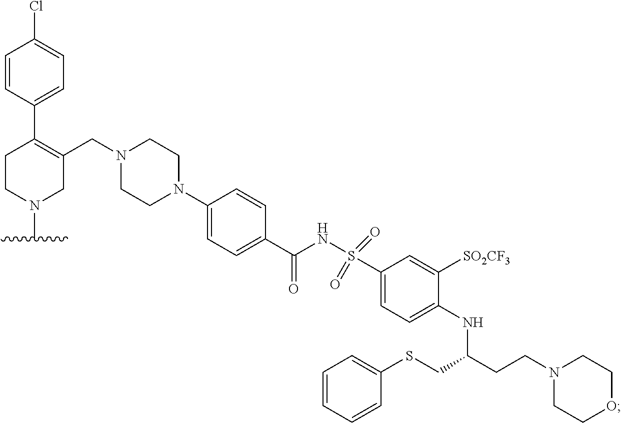

### and L₂ is

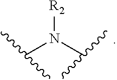

In another aspect, Y is

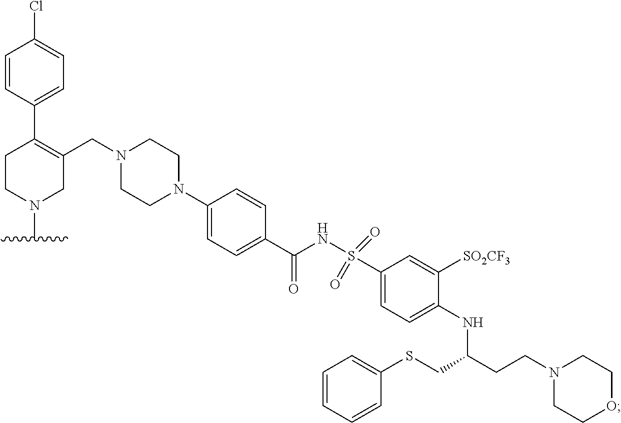

### L₂ is

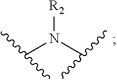

### and R is

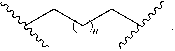

In another aspect, Y is

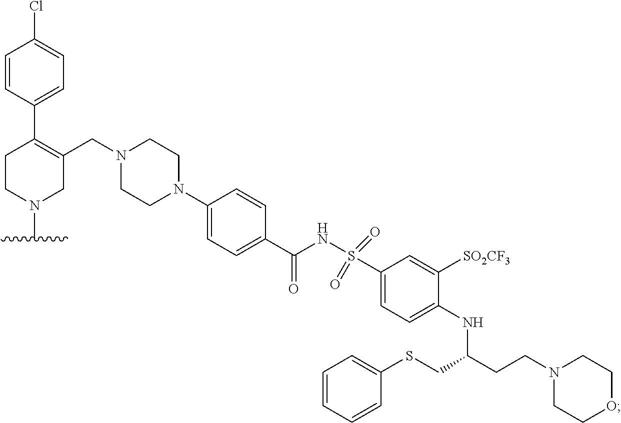

### L₂ is

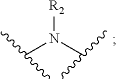

### R is

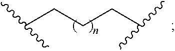

and n is 3-8, inclusive.

In another aspect, Y is

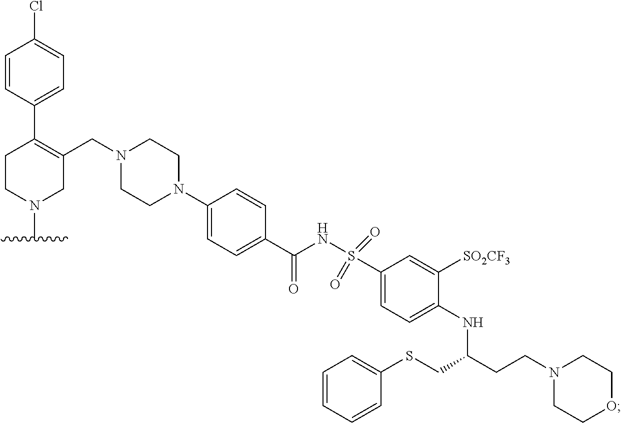

### L₂ is

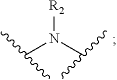

and L₁ is independently

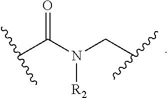

In another aspect, Y is

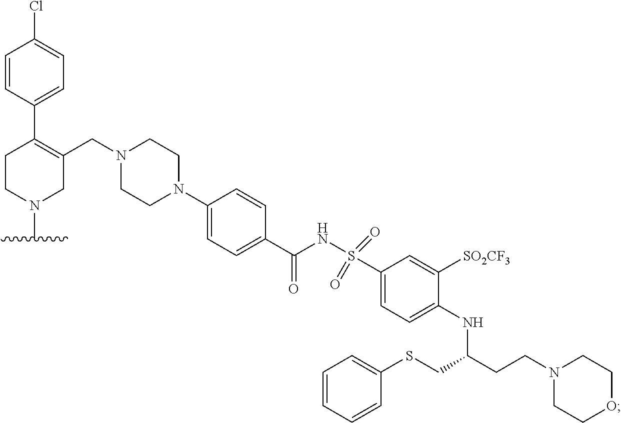

### L₂ is

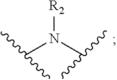

L₁ is independently

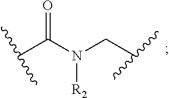

### and R is

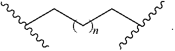

In another aspect, Y is

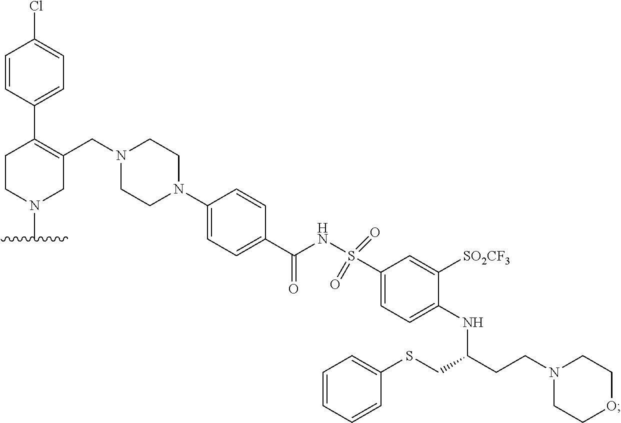

### L₂ is

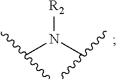

L₁ is independently

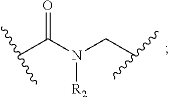

### R is

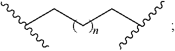

and n is 3-8, inclusive.

In another aspect, Y is

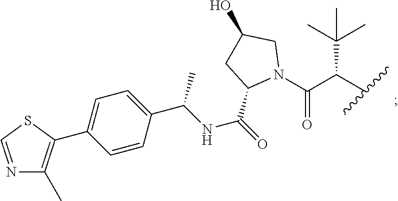 [SMILES: *[C@H](C(=O)N1C[C@H](O)C[C@H]1C(=O)N[C@@H](C)c1ccc(-c2scnc2C)cc1)C(C)(C)C]

### Y₂ is

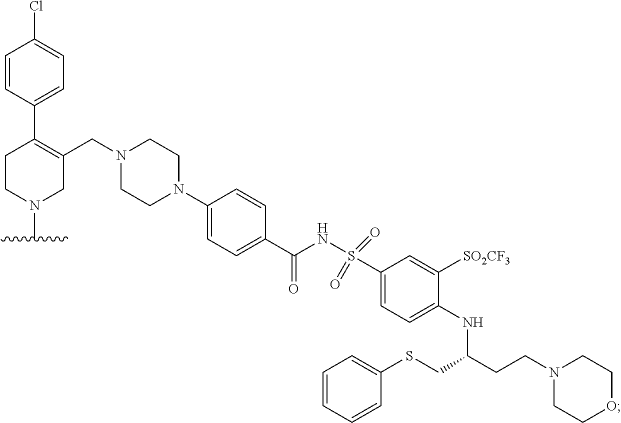

### and L₂ is

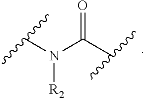

In another aspect, Y is

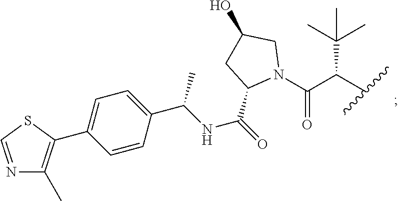 [SMILES: *[C@H](C(=O)N1C[C@H](O)C[C@H]1C(=O)N[C@@H](C)c1ccc(-c2scnc2C)cc1)C(C)(C)C]

### Y₂ is

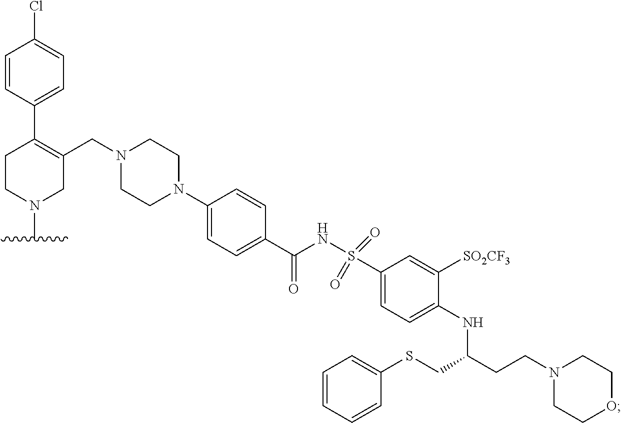

### L₂ is

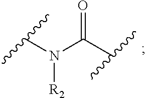

### and R is

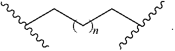

In another aspect, Y is

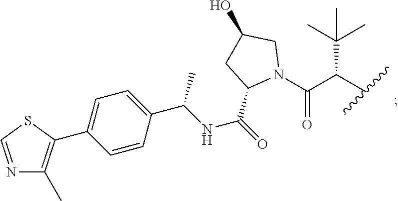 [SMILES: *[C@H](C(=O)N1C[C@H](O)C[C@H]1C(=O)N[C@@H](C)c1ccc(-c2scnc2C)cc1)C(C)(C)C]

### Y₂ is

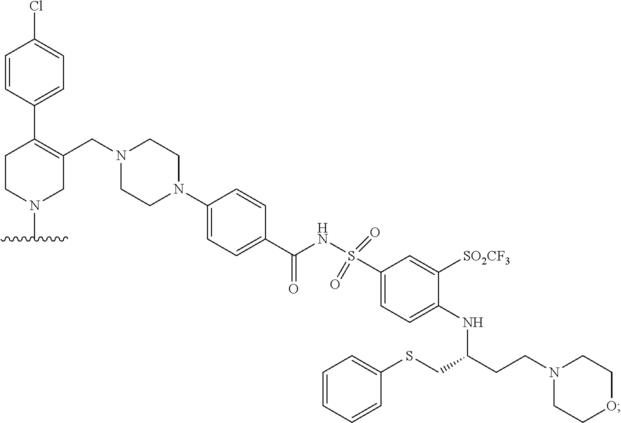

### L₂ is

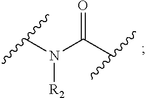

### R is

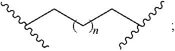

and n is 3-8, inclusive.

In another aspect, Y is

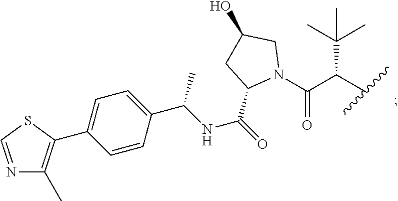 [SMILES: *[C@H](C(=O)N1C[C@H](O)C[C@H]1C(=O)N[C@@H](C)c1ccc(-c2scnc2C)cc1)C(C)(C)C]

### Y₂ is

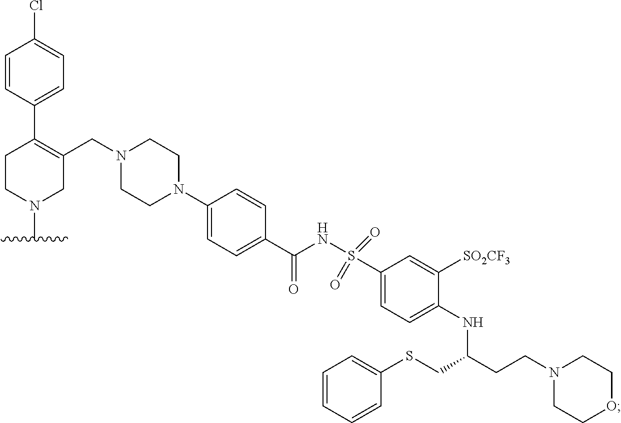

### L₂ is

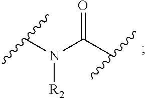

and L₁ is independently

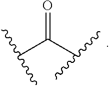 [SMILES: *C(*)=O]

In another aspect Y is

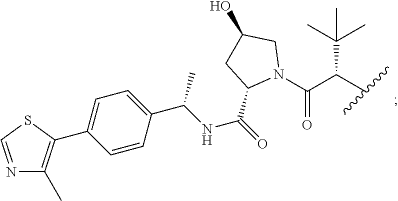 [SMILES: *[C@H](C(=O)N1C[C@H](O)C[C@H]1C(=O)N[C@@H](C)c1ccc(-c2scnc2C)cc1)C(C)(C)C]

### Y₂ is

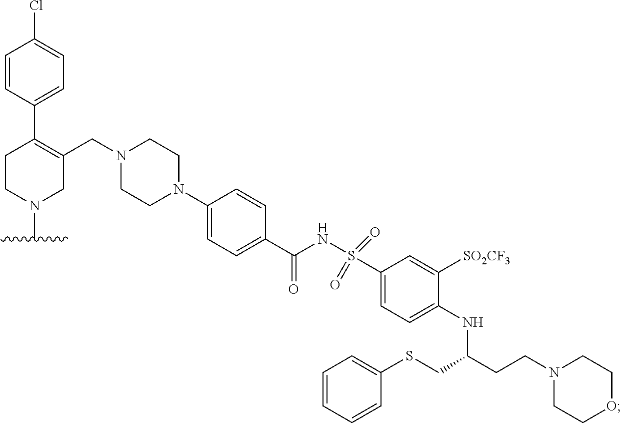

### L₂ is

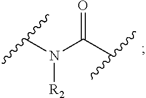

L₁ is independently

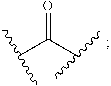 [SMILES: *C(*)=O]

### and R is

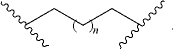

In another aspect, Y is

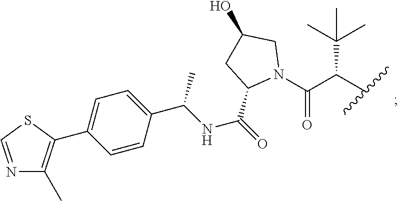 [SMILES: *[C@H](C(=O)N1C[C@H](O)C[C@H]1C(=O)N[C@@H](C)c1ccc(-c2scnc2C)cc1)C(C)(C)C]

### Y₂ is

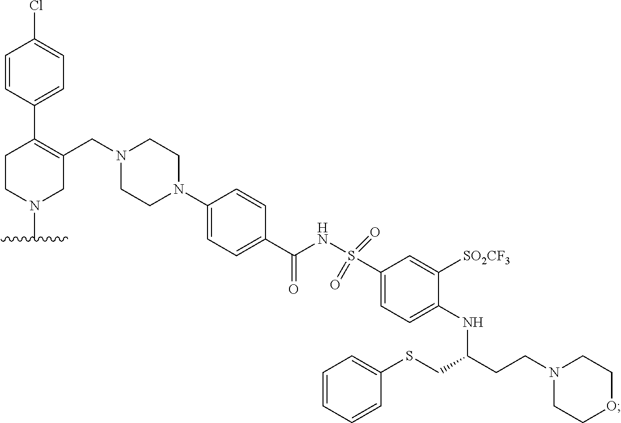

### L₂ is

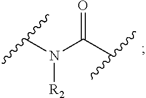

L₁ is independently

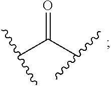 [SMILES: *C(*)=O]

### R is

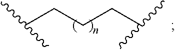

and n is 3-8, inclusive.

In another aspect, the compound of Formula (I) is:

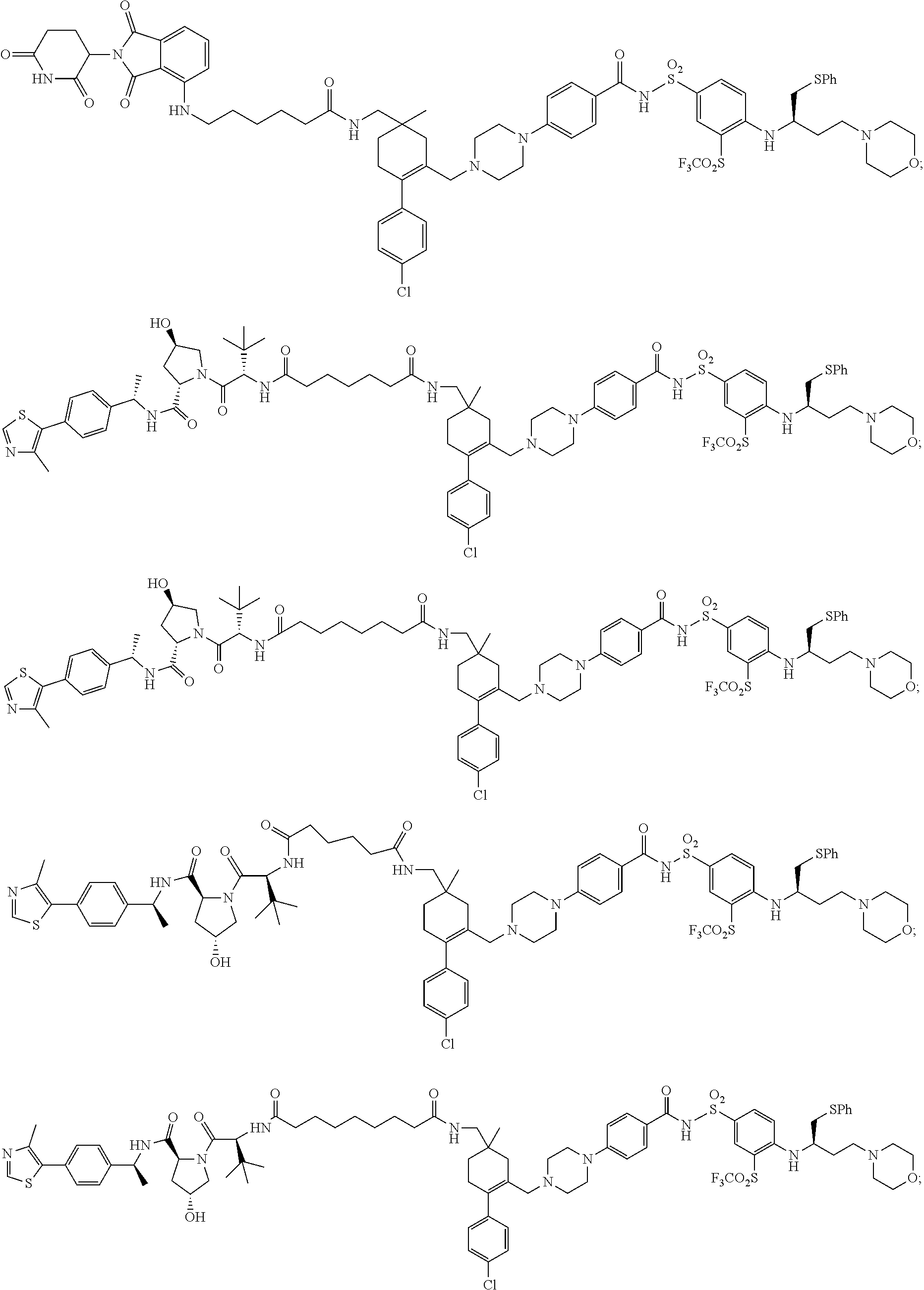

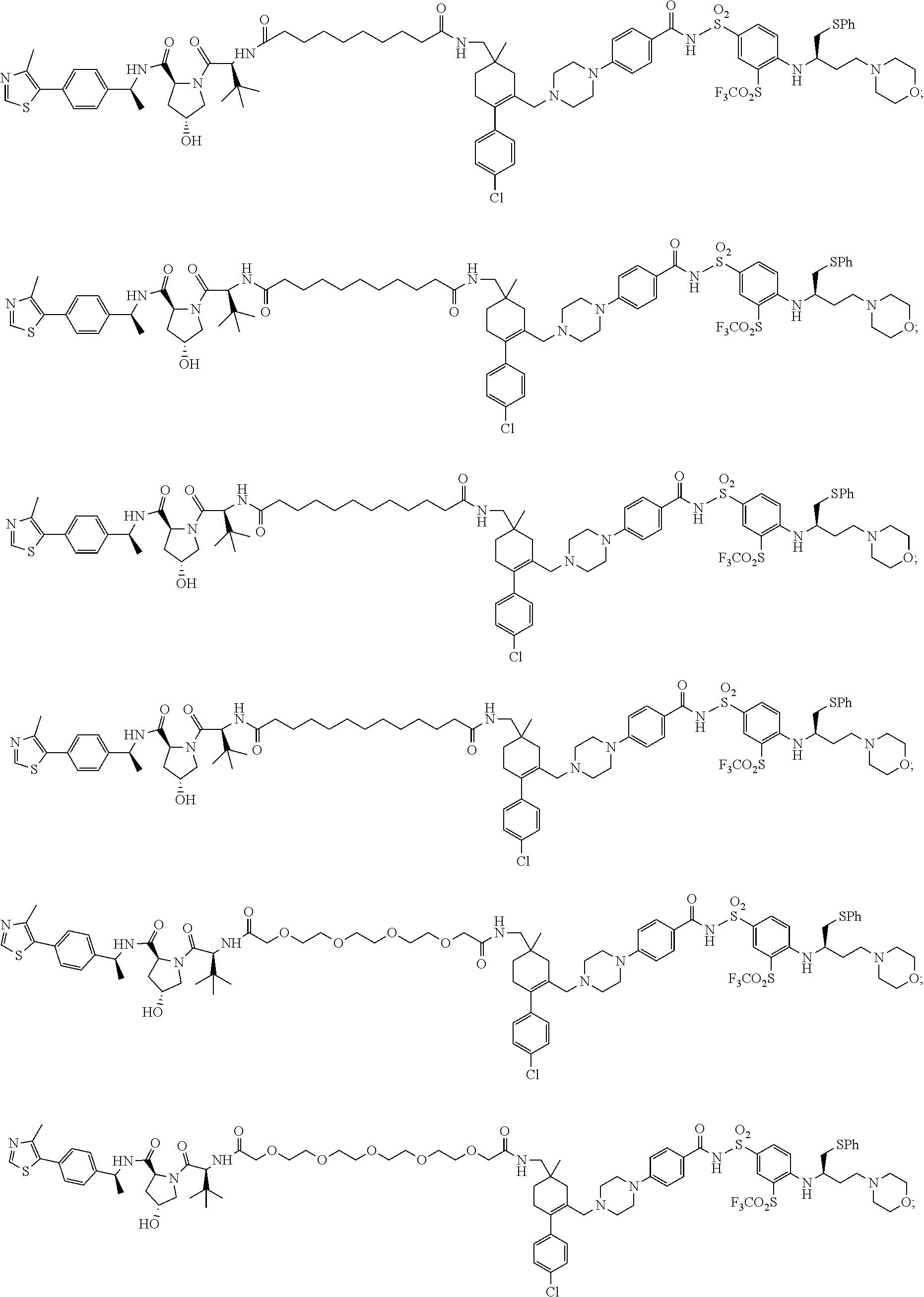

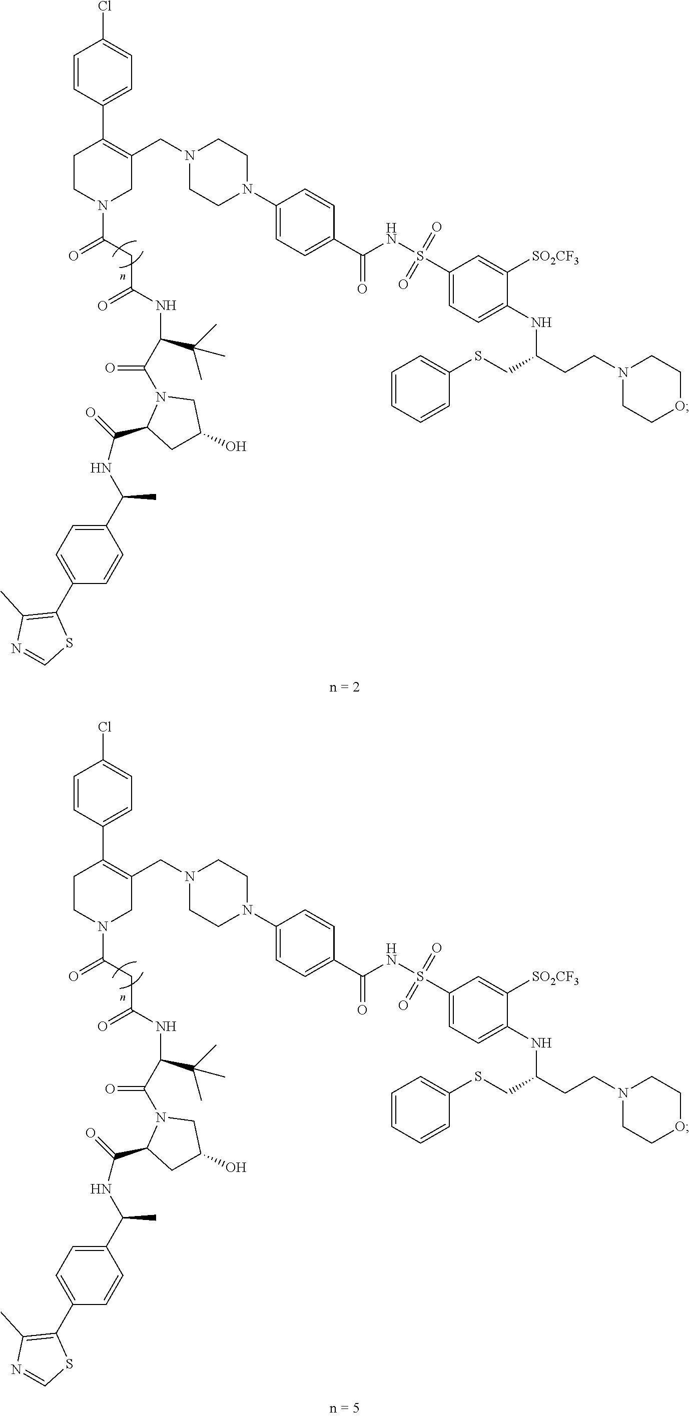

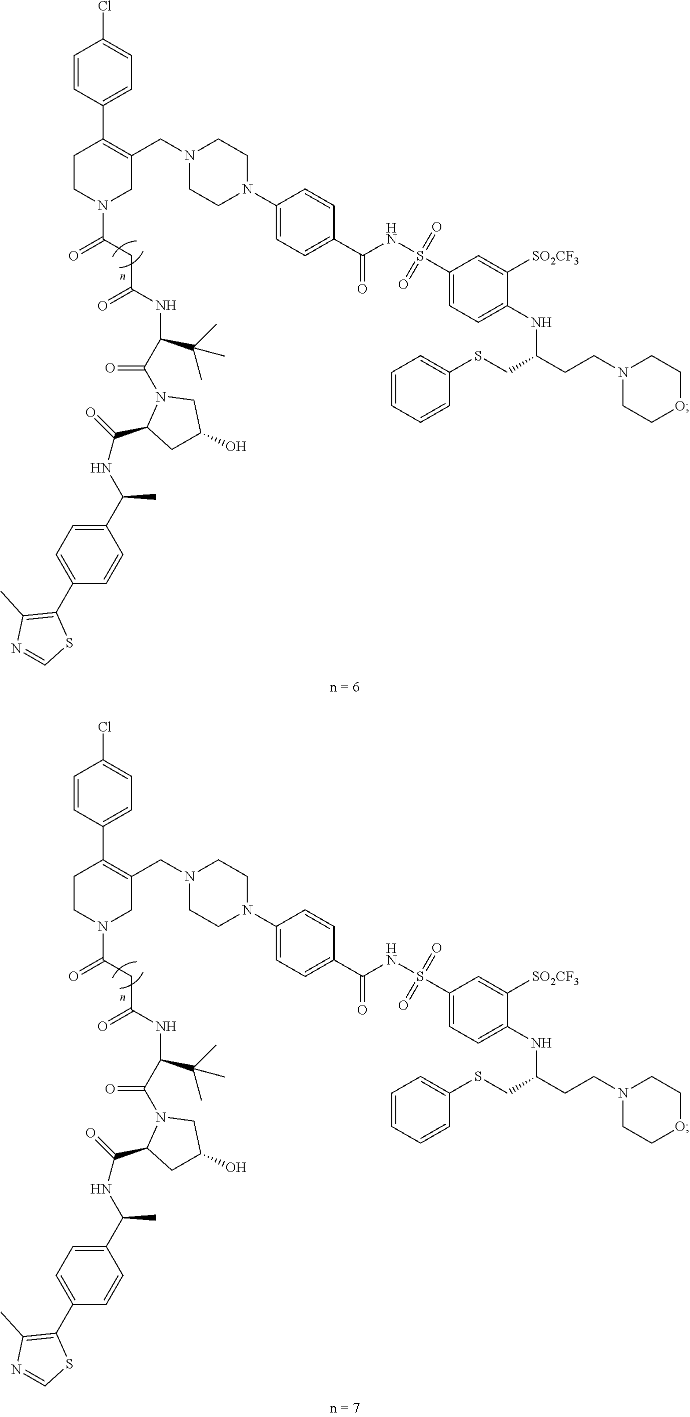

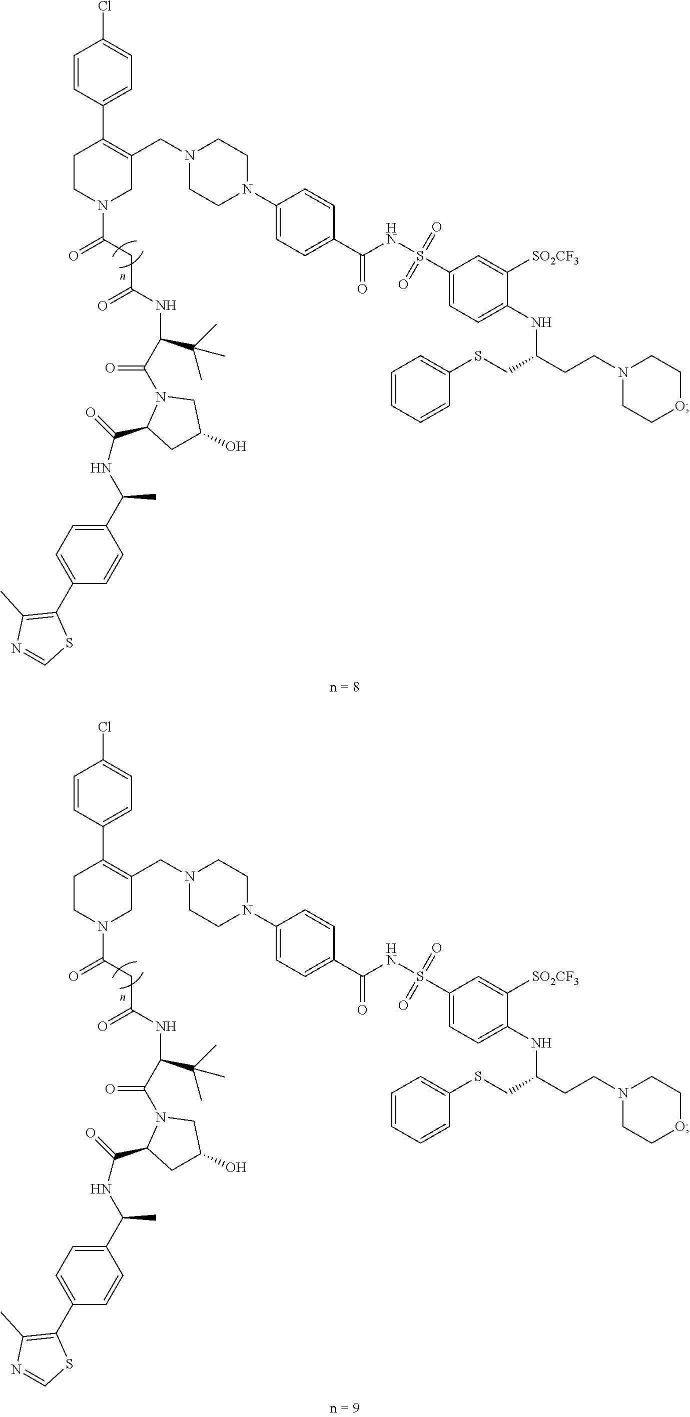

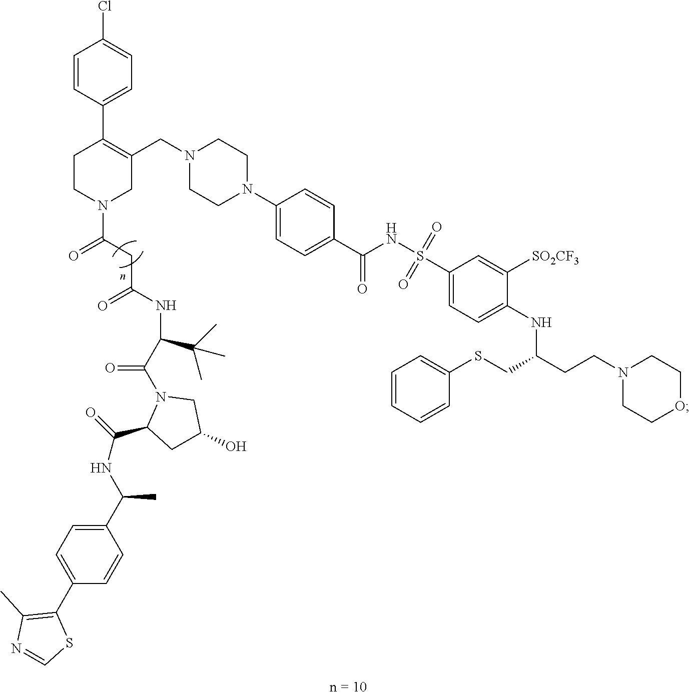

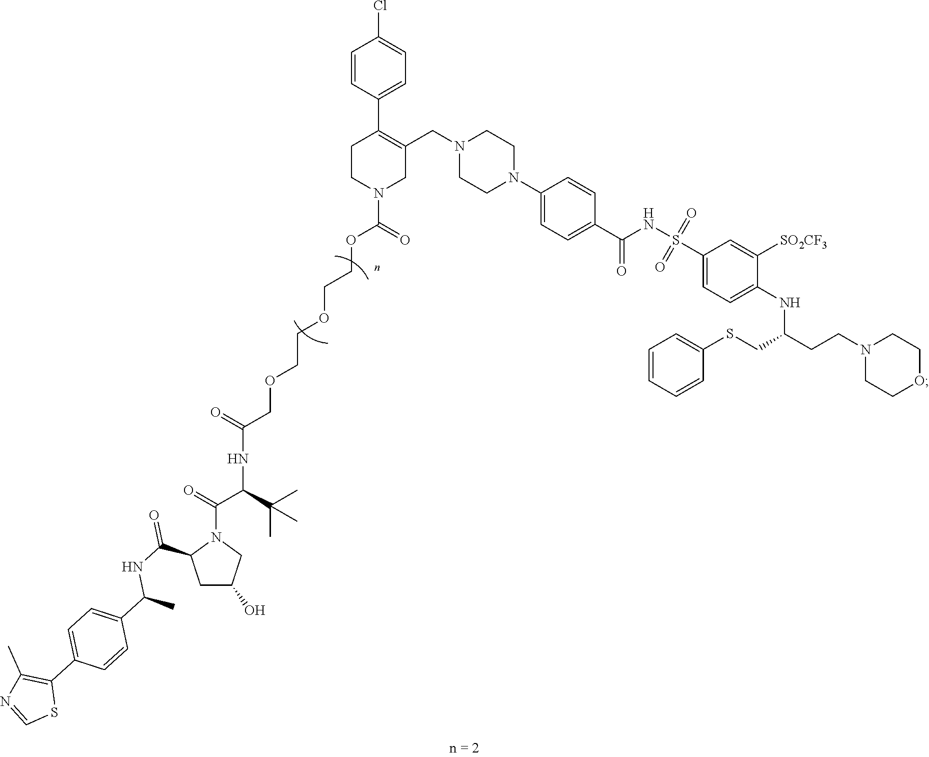

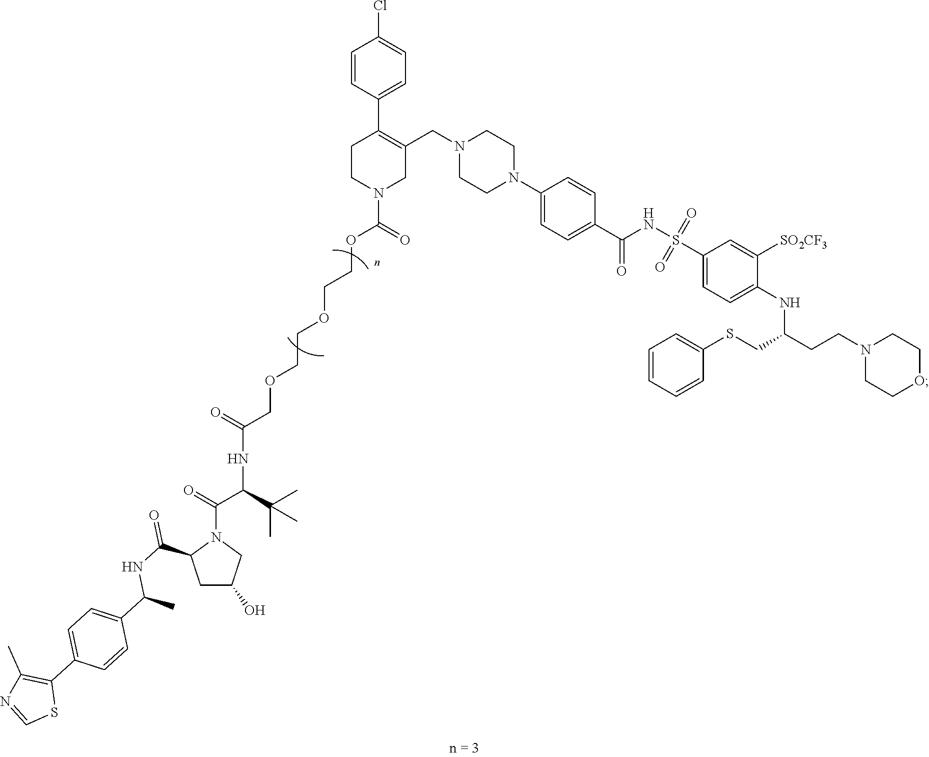

or a pharmaceutically acceptable salt, hydrate, solvate, or prodrug thereof.

In another aspect, the compound of Formula (I) is:

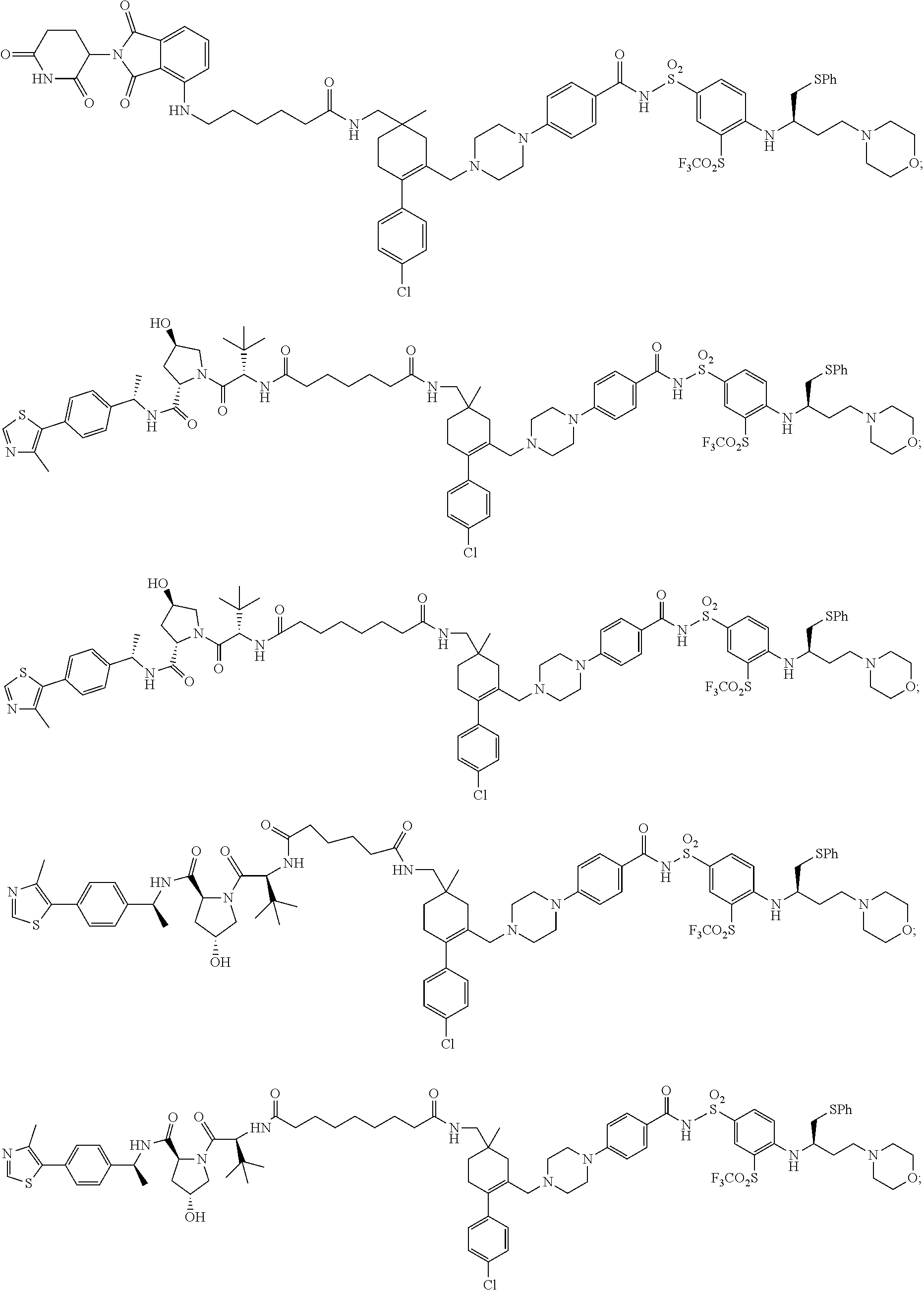

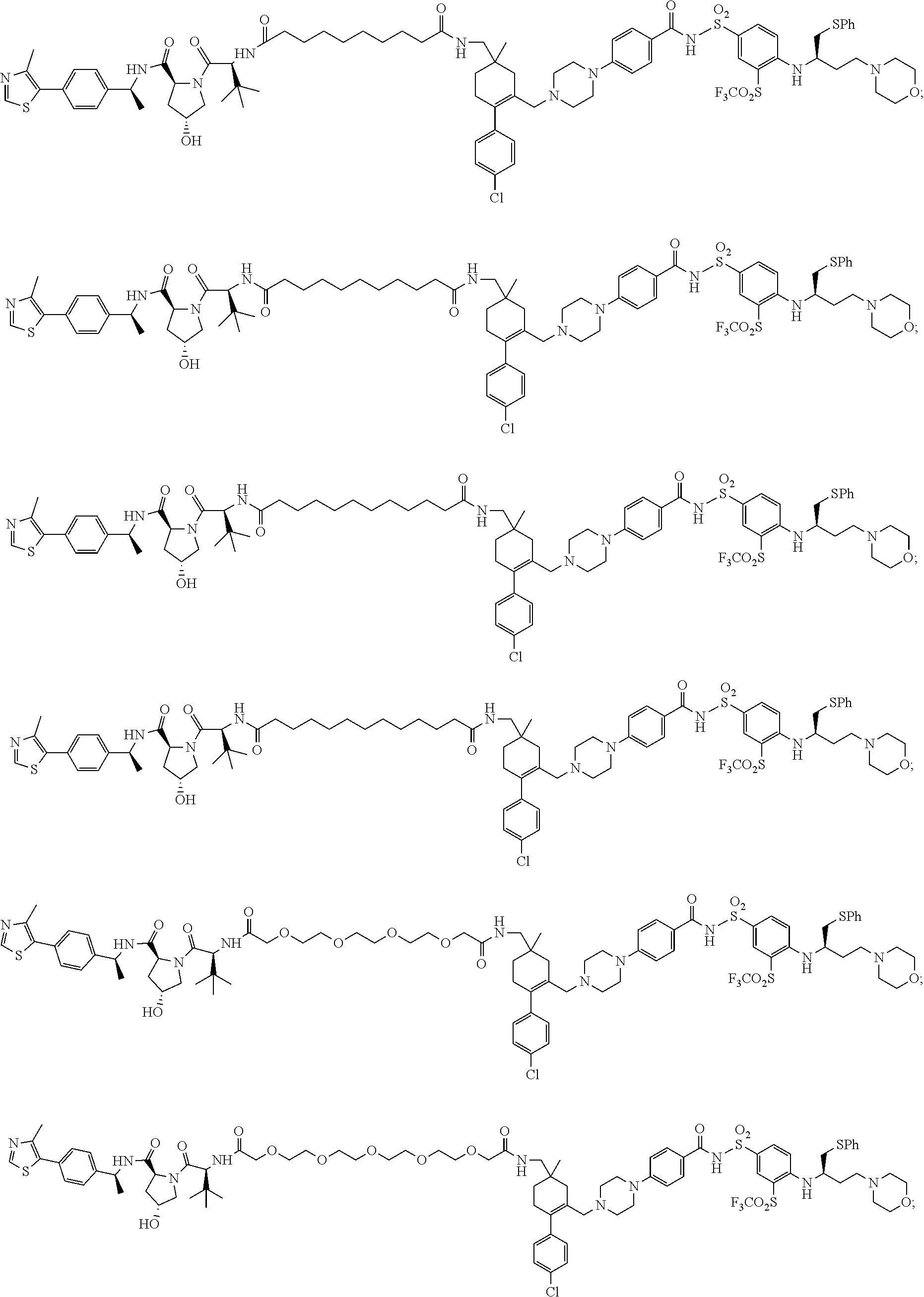

or a pharmaceutically acceptable salt, hydrate, solvate, or prodrug thereof.

In another aspect, the invention provides a compound of Table 3, or a pharmaceutically acceptable salt, hydrate, solvate, or prodrug thereof.

In another aspect, the invention provides a pharmaceutical composition comprising a compound described herein (e.g., Formula (I)), or a pharmaceutically acceptable salt, hydrate, solvate, or prodrug thereof, and a pharmaceutically acceptable carrier. In another aspect, the pharmaceutical composition of claim 34, further comprising an additional agent. In another aspect, the additional agent is an anti-cancer agent. In another aspect, the anti-cancer agent is an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

In another aspect, the invention provides a method of degrading Bcl-2 proteins, the method comprising administering an effective amount of a compound described herein (e.g., Formula (I)), or a pharmaceutically acceptable salt, hydrate, solvate, or prodrug thereof. In another aspect, the compound is administered in vitro. In another aspect, the compound is administered in vivo. In another aspect, the method further comprises administering the compound to a subject.

In another aspect, the invention provides a method of treating a disease or disorder in a subject in need thereof, the method comprising administering an effective amount of a compound described herein (e.g., Formula (I)), or a pharmaceutically acceptable salt, hydrate, solvate, or prodrug thereof. In another aspect, the disease is cancer. In another aspect, the cancer is a solid tumor. In another aspect, the cancer is chronic lymphocyctic leukemia. In another aspect, the subject is a mammal. In another aspect, the subject is a human.

In another aspect, the invention provides a method of treating a subject suffering from or susceptible to a disease or disorder, the method comprising administering an effective amount of a compound described herein (e.g., Formula (I)), or a pharmaceutically acceptable salt, hydrate, solvate, or prodrug thereof. In another aspect, the disease is cancer. In another aspect, the cancer is a solid tumor. In another aspect, the cancer is chronic lymphocyctic leukemia. In another aspect, the subject is a mammal. In another aspect, the subject is a human.

In another aspect, the invention provides a method of treating a Bcl-2-mediated cancer in a subject in need thereof, the method comprising administering an effective amount of a compound described herein (e.g., Formula (I)), or a pharmaceutically acceptable salt, hydrate, solvate, or prodrug thereof, wherein the platelet toxicity of the compound is less than other Bcl-2 inhibitors. In another aspect, the Bcl-2-mediated cancer is chronic lymphocyctic leukemia. In another aspect, the other Bcl-2 inhibitor is ABT-737, navitoclax (ABT-263), venetoclax (ABT-199), obatoclax (GX 15-070), (−)-gossypol (AT-101), sabutoclax (BI-97C1), TW-37, BM-1252 (APG-1252), or A-1155463. In another aspect, the other Bcl-2 inhibitor is venetoclax or ABT-263.

In another aspect, the invention provides a method of treating a subject suffering from or susceptible to a Bcl-2-mediated cancer, the method comprising administering an effective amount of a compound described herein (e.g., Formula (I)), or a pharmaceutically acceptable salt, hydrate, solvate, or prodrug thereof, wherein the platelet toxicity of the compound is less than other Bcl-2 inhibitors. In another aspect, the Bcl-2-mediated cancer is chronic lymphocyctic leukemia. In another aspect, the other Bcl-2 inhibitor is ABT-737, navitoclax (ABT-263), venetoclax (ABT-199), obatoclax (GX 15-070), (−)-gossypol (AT-101), sabutoclax (BI-97C1), TW-37, BM-1252 (APG-1252), or A-1155463. In another aspect, the other Bcl-2 inhibitor is venetoclax or ABT-263.

In another aspect, the invention provides a method of treating a Bcl-2-mediated cancer in a subject in need thereof, the method comprising administering an effective amount of a compound described herein (e.g., Formula (I)), or a pharmaceutically acceptable salt, hydrate, solvate, or prodrug thereof, such that ratio of human platelet toxicity (IC₅₀) to anticancer activity (IC₅₀) of the compound is greater than one. In another aspect, wherein the Bcl-2-mediated cancer is chronic lymphocyctic leukemia. In another aspect, wherein the anticancer activity is measured in MOLT-4 cells. In another aspect, wherein the ratio is greater than 2.5. In another aspect, wherein the ratio is greater than 5. In another aspect, wherein the ratio is greater than 10. In another aspect, wherein the ratio is greater than 20. In another aspect, wherein the ratio is greater than 40.

In another aspect, the invention provides a method of treating a subject suffering from or susceptible to a Bcl-2-mediated cancer, the method comprising administering an effective amount of a compound described herein (e.g., Formula (I)), or a pharmaceutically acceptable salt, hydrate, solvate, or prodrug thereof, such that ratio of human platelet toxicity (IC₅₀) to anticancer activity (IC₅₀) of the compound is greater than one. In another aspect, wherein the Bcl-2-mediated cancer is chronic lymphocyctic leukemia. In another aspect, wherein the anticancer activity is measured in MOLT-4 cells. In another aspect, wherein the ratio is greater than 2.5. In another aspect, wherein the ratio is greater than 5. In another aspect, wherein the ratio is greater than 10. In another aspect, wherein the ratio is greater than 20. In another aspect, wherein the ratio is greater than 40.

Compounds of the present invention are bivalent compounds that are able to promote the degradation of the anti-apoptotic Bcl-2 family of proteins. These bivalent compounds connect a Bcl-2 small molecule inhibitor or ligand to an E3 ligase binding moiety, such as von Hippel-Landau (VHL) E3 ligase binding moiety (such as HIF-1α-derived (R)-hydroxyproline containing VHL E3 ligase ligands) or cereblon (CRBN) E3 ligase binding moiety (thalidomide derivatives such as pomalidomide). VHL is part of the cullin-2 (CUL2) containing E3 ubiquitin ligase complex elongin BC-CUL2-VHL (known as CRL2VHL) responsible for degradation of the transcription factor HIF-1α. (R)-Hydroxyproline containing VHL E3 ligase ligands derived from HIF-1α have been identified with high affinity. CRBN is part of the cullin-4 (CUL4) containing E3 ubiquitin ligase complex CUL4-RBX1-DDB1-CRBN (known as CRL4CRBN). Thalidomide and its derivatives, such as lenalidomide and pomalidomide, interact specifically with this CRBN complex and induce degradation of essential IKAROS transcription factors. CC-122, a non-phthalimide analogue of thalidomide, also interacts with CRBN E3 ligase complex but induces the degradation of lymphoid transcription factor Aiolos. The bivalent compounds can actively recruit anti-apoptotic BcI-2 family of proteins to an E3 ubiquitin ligase, such as CRBN or VHL E3 ligase, resulting in their degradation by ubiquitin proteasome system.

Platelets depend on BcI-xL protein for survival. Thus, inhibition of BcI-xL protein in platelets causes thrombocytopenia which limits the use of Bcl-xL inhibitors as cancer therapeutic agents. Given the well-documented importance of Bcl-xL in solid tumors and its contribution to drug resistance, strategies devised to minimize the on-target platelet toxicity associated with the inhibition of BcI-xL could boost the therapeutic applications of drugs like ABT-263, a dual Bcl-2/Bcl-xL inhibitor, in cancer. The compounds in the present invention were designed to recruit an E3 ligase, such as CRBN or VHL E3 ligase, that is minimally expressed in platelets for the targeted degradation of BcI-xL.

Thus, the compounds described herein (e.g., Formula (I)) have reduced platelet toxicity compared with their corresponding Bcl-2/Bcl-xL inhibitors. Accordingly, the present disclosure provides compositions and methods for selectively degrading anti-apoptotic Bcl-2 family of proteins.

### BRIEF DESCRIPTION OF THE DRAWINGS

The present invention is further described below with reference to the following non-limiting examples and with reference to the following figures, in which:

FIG. 1. depicts the Western blotting analysis of Bcl-xL and apoptotic proteins 16 h after treatment with Compound 53 in MOLT-4 cells.

FIG. 2. depicts dose response curves of ABT-263 and Compound 53 in MOLT-4 T-ALL cells & human platelets determined by MTS assay.

FIG. 3. depicts the densitometric analysis of BCL-X_{L} degradation by Compound 53.

FIG. 4. depicts the ability of various compounds to form a ternary complex with the VHL complex and BCL-X_{L}.

FIG. 5. depicts the inability of the non-PROTAC compound shown to induce BCL-X_{L} degradation in Molt4 T-ALL cells.

FIG. 6. depicts the dose-dependent degradation of BCL-X_{L} in Molt4 T-ALL cells by Compound 26.

FIG. 7. depicts the inhibitory effects of degrader #5, degrader #41 and degrader #42 (the chiral pure diastereomers of degrader #5), and ABT-263 on MOLT-4, RS4; 11, NCI-H146 cells, and human platelets.

FIG. 8. shows degraders #5, #41, and #42 dose-dependently induced the degradation of Bcl-xL in MOLT-4 cells with DC₅₀ (concentration with 50% degradation) values of 21.5 nM, 100.5 nM, and 11.5 nM, respectively.

FIG. 9. shows degrader #5 did not affect Bcl-xL levels in human platelets.

FIG. 10. shows degraders #5 and #83 induced cleavage of caspase-3 and PARP in MOLT-4 cells after 16 h treatment.

FIG. 11. shows degraders #83, #84, and #85 formed ternary complexes with the VHL E3 ligase complex and Bcl-xL while their Bcl-xL binding portion (Bcl-xL ligand) did not.

### DETAILED DESCRIPTION

### Definitions

In order that the invention may be more readily understood, certain terms are first defined here for convenience.

As used herein, the term “treating” a disorder encompasses ameliorating, mitigating and/or managing the disorder and/or conditions that may cause the disorder. The terms “treating” and “treatment” refer to a method of alleviating or abating a disease and/or its attendant symptoms. In accordance with the present invention, “treating” includes blocking, inhibiting, attenuating, modulating, reversing the effects of and reducing the occurrence of e.g., the harmful effects of a disorder.

As used herein, “inhibiting” encompasses reducing and halting progression.

The term “modulate” refers to increases or decreases in the activity of a cell in response to exposure to a compound of the invention.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. Particularly, in embodiments the compound is at least 85% pure, more preferably at least 90% pure, more preferably at least 95% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “peptide” is a sequence of at least two amino acids. Peptides can consist of short as well as long amino acid sequences, including proteins.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “protein” refers to series of amino acid residues connected one to the other by peptide bonds between the alpha-amino and carboxy groups of adjacent residues.

Amino acids may be referred to herein by either their commonly known three letter symbols or by the one-letter symbols recommended by the IUPAC-IUB Biochemical Nomenclature Commission.

As to amino acid sequences, one of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “administration” or “administering” includes routes of introducing the compound(s) to a subject to perform their intended function. Examples of routes of administration which can be used include injection (subcutaneous, intravenous, parenterally, intraperitoneally, intrathecal), topical, oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the elastase inhibitor compound are outweighed by the therapeutically beneficial effects.

The phrases “systemic administration,” “administered systemically”, “peripheral administration” and “administered peripherally” as used herein mean the administration of a compound(s), drug or other material, such that it enters the patient's system and, thus, is subject to metabolism and other like processes.

The term “therapeutically effective amount” refers to that amount of the compound being administered sufficient to prevent development of or alleviate to some extent one or more of the symptoms of the condition or disorder being treated.

A therapeutically effective amount of compound (i.e., an effective dosage) may range from about 0.005 μg/kg to about 200 mg/kg, preferably about 0.1 mg/kg to about 200 mg/kg, more preferably about 10 mg/kg to about 100 mg/kg of body weight. In other embodiments, the therapeutically effect amount may range from about 1.0 pM to about 500 nM. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.005 μg/kg to about 200 mg/kg of body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound used for treatment may increase or decrease over the course of a particular treatment.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The term “subject” refers to animals such as mammals, including, but not limited to, primates (e.g., humans), cows, sheep, goats, horses, dogs, cats, rabbits, rats, mice and the like. In certain embodiments, the subject is a human.

Furthermore the compounds of the invention include olefins having either geometry: “Z” refers to what is referred to as a “cis” (same side) conformation whereas “E” refers to what is referred to as a “trans” (opposite side) conformation. With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

As used herein, the term “alkyl” refers to a straight-chained or branched hydrocarbon group containing 1 to 12 carbon atoms. The term “lower alkyl” refers to a C1-C6 alkyl chain. Examples of alkyl groups include methyl, ethyl, n-propyl, isopropyl, tert-butyl, and n-pentyl. Alkyl groups may be optionally substituted with one or more substituents.

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The sp² or sp carbons of an alkenyl group and an alkynyl group, respectively, may optionally be the point of attachment of the alkenyl or alkynyl groups.

The term “alkoxy” refers to an —O-alkyl radical.

As used herein, the term “halogen”, “hal” or “halo” means —F, —Cl, —Br or —I.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alkylating agents are any reagent that is capable of effecting the alkylation of the functional group at issue (e.g., oxygen atom of an alcohol, nitrogen atom of an amino group). Alkylating agents are known in the art, including in the references cited herein, and include alkyl halides (e.g., methyl iodide, benzyl bromide or chloride), alkyl sulfates (e.g., methyl sulfate), or other alkyl group-leaving group combinations known in the art. Leaving groups are any stable species that can detach from a molecule during a reaction (e.g., elimination reaction, substitution reaction) and are known in the art, including in the references cited herein, and include halides (e.g., I—, Cl—, Br—, F—), hydroxy, alkoxy (e.g., —OMe, —O-t-Bu), acyloxy anions (e.g., —OAc, —OC(O)CF₃), sulfonates (e.g., mesyl, tosyl), acetamides (e.g., —NHC(O)Me), carbamates (e.g., N(Me)C(O)Ot-Bu), phosphonates (e.g., —OP(O)(OEt)₂), water or alcohols (protic conditions), and the like.

In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy.

“Bcl-2” as used herein alone or as part of a group references to a member of the Bcl-2 family of proteins comprise the following Bcl-xL, MCL-1, Bcl-W, BFL-1/A1, Bcl-B, BAX, BAK, and BOK.

### Compounds of the Invention

Compounds delineated herein (i.e., Formula I) include salt, hydrate and solvates thereof. They include all compounds delineated in schemes herein, whether intermediate or final compounds in a process.

Compounds of the invention can be obtained from natural sources or made or modified made by means known in the art of organic synthesis. Methods for optimizing reaction conditions, if necessary minimizing competing by-products, are known in the art. Reaction optimization and scale-up may advantageously utilize high-speed parallel synthesis equipment and computer-controlled microreactors (e.g. Design And Optimization in Organic Synthesis, 2^{nd} Edition, Carlson R, Ed, 2005; Elsevier Science Ltd.; Jähnisch, K et al, Angew. Chem. Int. Ed. Engl. 2004 43: 406; and references therein). Additional reaction schemes and protocols may be determined by the skilled artesian by use of commercially available structure-searchable database software, for instance, SciFinder® (CAS division of the American Chemical Society) and CrossFire Beilstein® (Elsevier MDL), or by appropriate keyword searching using an internet search engine such as Google® or keyword databases such as the US Patent and Trademark Office text database. For example, compounds of formulae herein can be made using methodology known in the art, including Doi et al., Org Lett. 2006 Feb. 2; 8(3):531-4; Ma, et al., Chemistry. 2006 Oct. 10; 12(29):7615-26; and Chen et al., Proc Natl Acad Sci USA. 2004 Aug. 17; 101(33):12067-72.

The compounds herein may also contain linkages (e.g., carbon-carbon bonds) wherein bond rotation is restricted about that particular linkage, e.g. restriction resulting from the presence of a ring or double bond. Accordingly, all cis/trans and E/Z isomers are expressly included in the present invention. The compounds herein may also be represented in multiple tautomeric forms, in such instances, the invention expressly includes all tautomeric forms of the compounds described herein, even though only a single tautomeric form may be represented. All such isomeric forms of such compounds herein are expressly included in the present invention. All crystal forms and polymorphs of the compounds described herein are expressly included in the present invention. All hydrate and solvate forms of the compounds described herein are expressly included in the present invention. Also embodied are extracts and fractions comprising compounds of the invention. The term isomers is intended to include diastereoisomers, enantiomers, regioisomers, structural isomers, rotational isomers, tautomers, and the like. For compounds which contain one or more stereogenic centers, e.g., chiral compounds, the methods of the invention may be carried out with an enantiomerically enriched compound, a racemate, or a mixture of diastereomers.

Preferred enantiomerically enriched compounds have an enantiomeric excess of 50% or more, more preferably the compound has an enantiomeric excess of 60%, 70%, 80%, 90%, 95%, 98%, or 99% or more. In preferred embodiments, only one enantiomer or diastereomer of a chiral compound of the invention is administered to cells or a subject.

The compounds of the formulae herein can be synthesized using methodology similarly to that described in Chen, Q. Y.; Liu, Y.; Cai, W.; Luesch, H. Improved Total Synthesis and Biological Evaluation of Potent Apratoxin S4 Based Anticancer Agents with Differential Stability and Further Enhanced Activity. J. Med. Chem. 2014, 57 (7):p. 3011-302; and in WO2012/158933.

### Methods of Treatment

In another aspect, the invention provides a method of degrading Bcl-2 proteins, the method comprising administering an effective amount of a compound described herein (e.g., Formula (I)), or a pharmaceutically acceptable salt, hydrate, solvate, or prodrug thereof. In another aspect, the compound is administered in vitro. In another aspect, the compound is administered in vivo. In another aspect, the method further comprises administering the compound to a subject.

In another aspect, the invention provides a method of treating a disease or disorder in a subject in need thereof, the method comprising administering an effective amount of a compound described herein (e.g., Formula (I)), or a pharmaceutically acceptable salt, hydrate, solvate, or prodrug thereof. In another aspect, the disease is cancer. In another aspect, the cancer is a solid tumor. In another aspect, the cancer is chronic lymphocyctic leukemia. In another aspect, the subject is a mammal. In another aspect, the subject is a human.

In another aspect, the invention provides a method of treating a subject suffering from or susceptible to a disease or disorder, the method comprising administering an effective amount of a compound described herein (e.g., Formula (I)), or a pharmaceutically acceptable salt, hydrate, solvate, or prodrug thereof. In another aspect, the disease is cancer. In another aspect, the cancer is a solid tumor. In another aspect, the cancer is chronic lymphocyctic leukemia. In another aspect, the subject is a mammal. In another aspect, the subject is a human.

In another aspect, the invention provides a method of treating a Bcl-2-mediated cancer in a subject in need thereof, the method comprising administering an effective amount of a compound described herein (e.g., Formula (I)), or a pharmaceutically acceptable salt, hydrate, solvate, or prodrug thereof, such that platelet toxicity is reduced relative to other Bcl-2 inhibitors. In another aspect, the Bcl-2-mediated cancer is chronic lymphocyctic leukemia. In another aspect, the other Bcl-2 inhibitor is ABT-737, navitoclax (ABT-263), venetoclax (ABT-199), obatoclax (GX 15-070), (−)-gossypol (AT-101), sabutoclax (BI-97C1), TW-37, BM-1252 (APG-1252), or A-1155463. In another aspect, the other Bcl-2 inhibitor is venetoclax or ABT-263.

In another aspect, the invention provides a method of treating a subject suffering from or susceptible to a Bcl-2-mediated cancer, the method comprising administering an effective amount of a compound described herein (e.g., Formula (I)), or a pharmaceutically acceptable salt, hydrate, solvate, or prodrug thereof, such that platelet toxicity is reduced relative to other Bcl-2 inhibitors. In another aspect, the Bcl-2-mediated cancer is chronic lymphocyctic leukemia. In another aspect, the other Bcl-2 inhibitor is ABT-737, navitoclax (ABT-263), venetoclax (ABT-199), obatoclax (GX 15-070), (−)-gossypol (AT-101), sabutoclax (BI-97C1), TW-37, BM-1252 (APG-1252), or A-1155463. In another aspect, the other Bcl-2 inhibitor is venetoclax or ABT-263.

In another aspect, the invention provides a method of treating a Bcl-2-mediated cancer in a subject in need thereof, the method comprising administering an effective amount of a compound described herein (e.g., Formula (I)), or a pharmaceutically acceptable salt, hydrate, solvate, or prodrug thereof, such that ratio of human platelet toxicity (IC₅₀) to anticancer activity (IC₅₀) is less than that of other Bcl-2 inhibitors. In another aspect, wherein the Bcl-2-mediated cancer is chronic lymphocyctic leukemia. In another aspect, wherein the other Bcl-2 inhibitor is venetoclax or ABT-263. In another aspect, wherein the anticancer activity is measured in MOLT-4 cells. In another aspect, wherein the ratio is greater than 1. In another aspect, wherein the ratio is greater than 10. In another aspect, wherein the ratio is greater than 20. In another aspect, wherein the ratio is greater than 40.

In another aspect, the invention provides a method of treating a subject suffering from or susceptible to a Bcl-2-mediated cancer, the method comprising administering an effective amount of a compound described herein (e.g., Formula (I)), or a pharmaceutically acceptable salt, hydrate, solvate, or prodrug thereof, such that ratio of human platelet toxicity (IC₅₀) to anticancer activity (IC₅₀) is less than that of other Bcl-2 inhibitors. In another aspect, wherein the Bcl-2-mediated cancer is chronic lymphocyctic leukemia. In another aspect, wherein the other Bcl-2 inhibitor is venetoclax or ABT-263. In another aspect, wherein the anticancer activity is measured in MOLT-4 cells. In another aspect, wherein the ratio is greater than 1. In another aspect, wherein the ratio is greater than 10. In another aspect, wherein the ratio is greater than 20. In another aspect, wherein the ratio is greater than 40.

The present disclosure encompasses a method of selectively killing one or more cancer cells in a sample, the method comprising contacting a composition comprising an effective amount of a compound of Formula (I) with the sample. In another aspect, the present disclosure encompasses a method of selectively killing one or more cancer cells in a subject in need thereof, the method comprising administering to the subject a composition comprising a therapeutically effective amount of a compound of Formula (I).

By selectively killing one or more cancer cells is meant a composition of the invention does not appreciably kill non-cancer cells at the same concentration. In one embodiment, a composition of the invention has reduced platelet toxicity and retained or improved toxicity in cancer cells when compared to similar BCL-2 inhibitors. Accordingly, the median lethal dose or LD50 of the inhibitor in non-cancer cells may be about 5 to about 50 times higher than the LD50 of the inhibitor in cancer cells. As used herein, the LD50 is the concentration of inhibitor required to kill half the cells in the cell sample. For example, the LD50 of the inhibitor in non-cancer cells may be greater than about 5, about 6, about 7, about 8, about 9 or about 10 times higher than the LD50 of the inhibitor in cancer cells. Alternatively, the LD50 of the inhibitor in non-cancer cells may be greater than about 10, about 15, about 20, about 25, about 30, about 35, about 40, about 45, or about 50 times higher than the LD50 of the inhibitor in cancer cells. Additionally, the LD50 of the inhibitor in non-cancer cells may be greater than 50 times higher than the LD50 of the inhibitor in cancer cells. In a specific embodiment, the LD50 of the inhibitor in non-cancer cells is greater than 10 times higher than the LD500 of the inhibitor in cancer cells. In another specific embodiment, the LD50 of the inhibitor in non-cancer cells is greater than 20 times higher than the LD50 of the inhibitor in cancer cells.

Non-limiting examples of neoplasms or cancers that may be treated include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas, breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, choriocarcinoma, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, glioblastoma, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood). In certain embodiments, a cancer is selected from the group consisting of synovial sarcoma, Burkitt lymphoma, Hodgkin lymphoma, multiple myeloma, neuroblastoma, glioblastoma, small cell lung cancer, pancreatic cancer, hepatocellular (liver) cancer, endometrial cancer, ovarian cancer, cervical cancer, breast cancer, prostate cancer, bladder cancer, melanoma, rhabdomyosarcoma, osteosarcoma/malignant fibrous histiocytoma of bone, choriocarcinoma, kidney cancer (renal cell cancer), thyroid cancer, and leukemias (acute lymphoblastic, acute myeloid, chronic lymphocytic, and chronic myelogenous).

### Pharmaceutical Compositions

In one aspect, the invention provides a pharmaceutical composition comprising the compound of any of the formulae herein (e.g., Formula (I)), and a pharmaceutically acceptable carrier.

In another embodiment, the invention provides a pharmaceutical composition wherein the compound of any of the formulae herein is a compound of any of Formula I and a pharmaceutically acceptable carrier. In another aspect, the composition further comprises an additional agent. In another aspect, the additional agent is an anti-cancer agent. In another aspect, the anticancer agent is alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

Non-limiting examples of suitable alkylating agents include altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lipoplatin, lomustine (CCNU), mafosfamide, mannosulfan, mechlorethamine, mechlorethamine oxide hydrochloride, melphalan, meturedopa, mustine (mechlorethamine), mitobronitol, nimustine, novembichin, oxaliplatin, phenesterine, piposulfan, prednimustine, ranimustine, satraplatin, semustine, temozolomide, thiotepa, treosulfan, triaziquone, triethylenemelamine, triethylenephosphoramide (TEPA), triethylenethiophosphaoramide (thiotepa), trimethylolomelamine, trofosfamide, uracil mustard and uredopa.

Suitable anti-metabolites include, but are not limited to aminopterin, ancitabine, azacitidine, 8-azaguanine, 6-azauridine, capecitabine, carmofur (1-hexylcarbomoyl-5-fluorouracil), cladribine, clofarabine, cytarabine (cytosine arabinoside (Ara-C)), decitabine, denopterin, dideoxyuridine, doxifluridine, enocitabine, floxuridine, fludarabine, 5-fluorouracil, gemcetabine, hydroxyurea (hydroxycarbamide), leucovorin (folinic acid), 6-mercaptopurine, methotrexate, nafoxidine, nelarabine, oblimersen, pemetrexed, pteropterin, raltitrexed, tegofur, tiazofurin, thiamiprine, tioguanine (thioguanine), and trimetrexate.

Non-limiting examples of suitable anti-tumor antibiotics include aclacinomysin, aclarubicin, actinomycins, adriamycin, aurostatin (for example, monomethyl auristatin E), authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, epoxomicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, sparsomycin, streptonigrin, streptozocin, tubercidin, valrubicin, ubenimex, zinostatin, and zorubicin.

Non-limiting examples of suitable anti-cytoskeletal agents include cabazitaxel, colchicines, demecolcine, docetaxel, epothilones, ixabepilone, macromycin, omacetaxine mepesuccinate, ortataxel, paclitaxel (for example, DHA-paclitaxel), taxane, tesetaxel, vinblastine, vincristine, vindesine, and vinorelbine.

Suitable topoisomerase inhibitors include, but are not limited to, amsacrine, etoposide (VP-16), irinotecan, mitoxantrone, RFS 2000, teniposide, and topotecan.

Non-limiting examples of suitable anti-hormonal agents such as aminoglutethimide, antiestrogen, aromatase inhibiting 4(5)-imidazoles, bicalutamide, finasteride, flutamide, fluvestrant, goserelin, 4-hydroxytamoxifen, keoxifene, leuprolide, LY117018, mitotane, nilutamide, onapristone, raloxifene, tamoxifen, toremifene, and trilostane.

Examples of targeted therapeutic agents include, without limit, monoclonal antibodies such as alemtuzumab, cartumaxomab, edrecolomab, epratuzumab, gemtuzumab, gemtuzumab ozogamicin, glembatumumab vedotin, ibritumomab tiuxetan, reditux, rituximab, tositumomab, and trastuzumab; protein kinase inhibitors such as bevacizumab, cetuximab, crizonib, dasatinib, erlotinib, gefitinib, imatinib, lapatinib, mubritinib, nilotinib, panitumumab, pazopanib, sorafenib, sunitinib, toceranib, and vandetanib;

Angiogeneisis inhibitors such as angiostatin, bevacizumab, denileukin diftitox, endostatin, everolimus, genistein, interferon alpha, interleukin-2, interleukin-12, pazopanib, pegaptanib, ranibizumab, rapamycin (sirolimus), temsirolimus, and thalidomide; and growth inhibitory polypeptides such as bortazomib, erythropoietin, interleukins (e.g., IL-1, IL-2, IL-3, IL-6), leukemia inhibitory factor, interferons, romidepsin, thrombopoietin, TNF-α, CD30 ligand, 4-1BB ligand, and Apo-1 ligand.

Non-limiting examples of photodynamic therapeutic agents include aminolevulinic acid, methyl aminolevulinate, retinoids (alitretinon, tamibarotene, tretinoin), and temoporfin.

Other antineoplastic agents include anagrelide, arsenic trioxide, asparaginase, bexarotene, bropirimine, celecoxib, chemically linked Fab, efaproxiral, etoglucid, ferruginol, lonidamide, masoprocol, miltefosine, mitoguazone, talapanel, trabectedin, and vorinostat.

In one aspect, the invention provides a kit comprising an effective amount of a compound of any of the formulae herein (e.g., Formula (I)), in unit dosage form, together with instructions for administering the compound to a subject suffering from or susceptible to cancer. In another aspect, the cancer is a solid tumor. In another aspect, the cancer is chronic lymphocyctic leukemia.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The invention also provides a pharmaceutical composition, comprising an effective amount a compound described herein and a pharmaceutically acceptable carrier. In an embodiment, compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic (or unacceptably toxic) to the patient.

In use, at least one compound according to the present invention is administered in a pharmaceutically effective amount to a subject in need thereof in a pharmaceutical carrier by intravenous, intramuscular, subcutaneous, or intracerebro ventricular injection or by oral administration or topical application. In accordance with the present invention, a compound of the invention may be administered alone or in conjunction with a second, different therapeutic. By “in conjunction with” is meant together, substantially simultaneously or sequentially. In one embodiment, a compound of the invention is administered acutely. The compound of the invention may therefore be administered for a short course of treatment, such as for about 1 day to about 1 week. In another embodiment, the compound of the invention may be administered over a longer period of time to ameliorate chronic disorders, such as, for example, for about one week to several months depending upon the condition to be treated.

By “pharmaceutically effective amount” as used herein is meant an amount of a compound of the invention, high enough to significantly positively modify the condition to be treated but low enough to avoid serious side effects (at a reasonable benefit/risk ratio), within the scope of sound medical judgment. A pharmaceutically effective amount of a compound of the invention will vary with the particular goal to be achieved, the age and physical condition of the patient being treated, the severity of the underlying disease, the duration of treatment, the nature of concurrent therapy and the specific apratoxin compound employed. For example, a therapeutically effective amount of a compound of the invention administered to a child or a neonate will be reduced proportionately in accordance with sound medical judgment. The effective amount of a compound of the invention will thus be the minimum amount which will provide the desired effect.

The compound may be administered parenterally or intraperitoneally. Dispersions can also be prepared, for example, in glycerol, liquid polyethylene glycols, and mixtures thereof, and in oils.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage. The carrier can be a solvent or dispersion medium containing, for example, water, DMSO, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the compound of the invention in the required amount in the appropriate solvent with various of the other ingredients enumerated above, as required, followed by filtered sterilization. Generally, dispersions are prepared by incorporating the various sterilized compounds into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yields a powder of the active ingredient plus any additional desired ingredient from previously sterile-filtered solution thereof.

For oral therapeutic administration, the compound may be incorporated with excipients and used in the form of ingestible tablets, buccal tablets, troches, capsules, elixirs, suspensions, syrups, wafers, and the like. Compositions or preparations according to the present invention are prepared so that an oral dosage unit form contains compound concentration sufficient to treat a disorder in a subject.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### EXAMPLES

The present invention will now be demonstrated using specific examples that are not to be construed as limiting.

### Compound Preparation

### Preparation of 9: tert-butyl (R)-4-(4-chlorophenyl)-5-((4-(4-(((4-((4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridine-1(2H)-carboxylate (9)

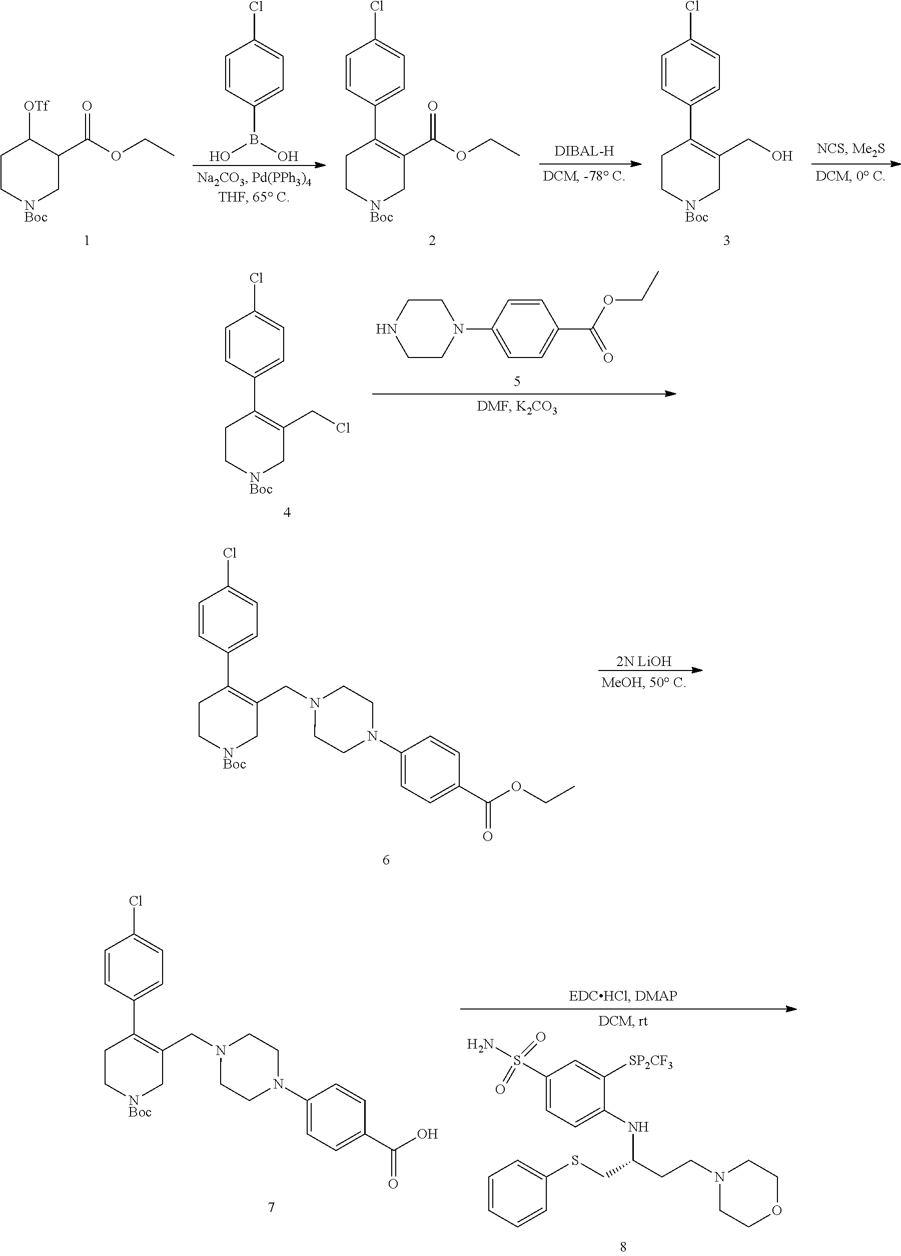

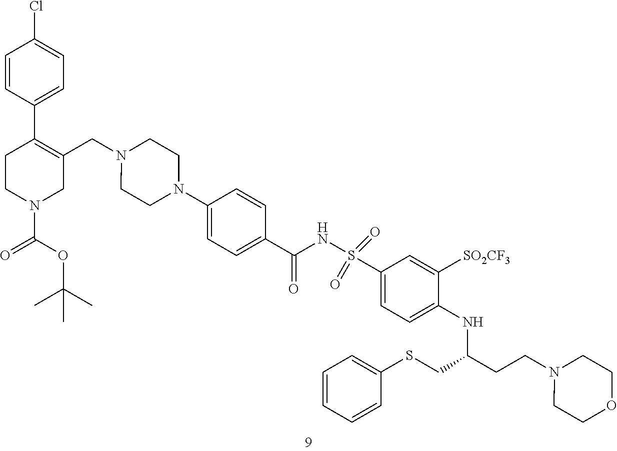

Synthesis of 1-(tert-butyl) 3-ethyl 4-(4-chlorophenyl)-5,6-dihydropyridine-1,3(2H)-dicarboxylate (2): To a solution of trifluoromethanesulfonate 1 (200 mg, 0.5 mmol) and 4-chlorophenylboronic acid (93 mg, 0.6 mmol) in THE (3.4 mL) was added aqueous solution of Na₂CO₃ (2.0 M, 0.77 mL). The resulting mixture was degassed by purging N₂. Pd(PPh₃)₄ (10 mg, 0.0087 mmol) was then added and the mixture was heated to 65° C. for 3 h. The mixture was filtered through a pad of celite. The filtrate was diluted with ethyl acetate and washed with water and then brine. The organic layer was collected, dried over sodium sulfate, filtered, and condensed under reduced pressure to afford the title compound (150 mg, 83% yield). ¹H NMR (600 MHz, CDCl₃) δ 7.30 (d, J=8.5 Hz, 2H), 7.06 (d, J=8.5 Hz, 2H), 4.24 (s, 2H), 3.96 (q, J=7.1 Hz, 2H), 3.60 (t, J=5.6 Hz, 2H), 2.46 (s, 2H), 1.50 (s, 9H), 0.97 (s, 3H) ppm.

Synthesis of tert-butyl 4-(4-chlorophenyl)-5-(hydroxymethyl)-3,6-dihydropyridine-1(2H)-carboxylate (3): To a solution of compound 2 (80 mg, 0.22 mmol) in THF (2 mL) at −78° C. was added DIBAL-H solution (1.2 M in tolulene, 0.73 mL, 0.88 mmol). The resulting mixture was stirred at −78° C. for 2-3 h. Several drops of methanol were added to quench the reaction. After warming to room temperature, the mixture was diluted with ethyl acetate and poured into 10 mL saturated Rochelle solution. After stirring at room temperature overnight, the mixture was well layered. The organic phase was collected and washed with water and brine, dried over sodium sulfate, filtered, and condensed to afford a residue which was purified by silica gel column flash chromatography (ethyl acetate/Hexanes 5:1-3:1) to yield the title compound (60 mg, 86%). ¹H NMR (600 MHz, CDCl₃) δ 7.33-7.28 (m, 2H), 7.16-7.07 (m, 2H), 4.11 (s, 2H), 4.00 (s, 2H), 3.58 (t, J=5.7 Hz, 2H), 2.37 (s, 2H), 1.48 (s, 9H) ppm.

Synthesis of tert-butyl tert-butyl 5-(chloromethyl)-4-(4-chlorophenyl)-3,6-dihydropyridine-1(2H)-carboxylate (4): To a stirring solution of NCS (83 mg, 0.62 mmol) in dry DCM (1 mL) was added Me₂S (50 μL, 0.68 mmol) at 0° C. A solution of compound 3 (100 mg, 0.31 mmol) in DCM (0.5 mL) was then added dropwise. The resulting mixture was stirred at 0° C. until fully conversion of alcohol compound (approximately 1 h). Water was added to quench the reaction, and the mixture was then extracted with ethyl acetate for 3 times. The combined organic phases were washed with brine, dried over sodium sulfate, filtered, and condensed under reduced pressure to afford a residue which was chromatographed on silica gel (4:1 hexanes/ethyl acetate) to yield the chloride product (100 mg, 95% yield) ¹H NMR (600 MHz, CDCl₃) δ 7.37-7.33 (m, 2H), 7.22-7.13 (m, 2H), 4.11 (s, 2H), 3.93 (s, 2H), 3.60 (t, J=5.6 Hz, 2H), 2.41 (s, 2H), 1.50 (s, 9H) ppm.

Synthesis of tert-butyl 4-(4-chlorophenyl)-5-((4-(4-(ethoxycarbonyl)phenyl)piperazin-1-yl)methyl)-3,6-dihydropyridine-1(2H)-carboxylate (6): To a stirring solution of compound 4 (50 mg, 0.15 mmol) in DMF was added compound 5 (34.4 mg, 0.15 mmol) and Cs₂CO₃ (95 mg, 0.29 mmol). After stirring at room temperature for 1.5 h, water was added, and the mixture was extracted with ethyl acetate for 3 times. The combined organic phases were washed with water and brine, dried over sodium sulfate, filtered, and condensed under reduced pressure to afford a residue which was chromatographed on silica gel (5:1 hexanes/ethyl acetate) to yield the title compound (40 mg, 51% yield). ¹H NMR (600 MHz, CDCl₃) δ 7.90 (d, J=8.9 Hz, 2H), 7.30 (d, J=8.3 Hz, 2H), 7.03 (d, J=8.3 Hz, 2H), 6.81 (d, J=8.8 Hz, 2H), 4.32 (q, J=7.1 Hz, 2H), 4.07 (s, 2H), 3.59 (t, J=5.2 Hz, 2H), 3.32-3.22 (m, 4H), 2.90 (s, 2H), 2.38 (dd, J=11.3, 6.4 Hz, 6H), 1.50 (s, 9H), 1.36 (t, J=7.1 Hz, 3H) ppm; ESI m/z 534.2 (M+H)⁺.

Synthesis of 4-(4-((1-(tert-butoxycarbonyl)-4-(4-chlorophenyl)-1,2,5,6-tetrahydropyridin-3-yl)methyl)piperazin-1-yl)benzoic acid (7): To a stirring solution of compound 6 (200 mg, 0.37 mmol) in methanol (3 mL) was added LiOH aqueous (2 N, 1 mL). The resulting mixture was heated to 55° C. and stirred at this temperature for 3 h. Upon cool down to room temperature, the pH of the mixture was adjust to 7 with 3N HCl. The mixture was then extracted with ethyl acetate and the combined organic layers were washed with brine, dried over sodium sulphate, and condensed to afford a residue which was chromatographed on silical gel (3:1 hexanes/ethyl acetate) to afford the title compound (180 mg, 95% yield). ¹H NMR (600 MHz, CDCl₃) δ 7.95 (d, J=8.7 Hz, 2H), 7.33 (d, J=8.1 Hz, 2H), 7.07-7.00 (m, 2H), 6.81 (d, J=8.6 Hz, 2H), 4.11 (s, 2H), 3.60 (t, J=5.4 Hz, 2H), 3.35 (s, 4H), 3.07 (s, 2H), 2.8-2.15 (m, 6H), 1.49 (s, 9H) ppm; ESI m/z 512.2 (M+H)⁺.

Synthesis of tert-butyl (R)-4-(4-chlorophenyl)-5-((4-(4-(((4-((4-morpholino-1-(phenylthio) butan-2-yl)amino)-3-((trifluoromethyl)sulfonyl)phenyl)sulfonyl)carbamoyl) phenyl) piperazin-1-yl)methyl)-3,6-dihydropyridine-1(2H)-carboxylate (9): To a stirring solution of compound 7 (100 mg, 0.2 mmol) in DCM (2.5 mL) was added (R)-4-((4-morpholino-1-(phenylthio)butan-2-yl)amino)-3-((trifluoromethyl)sulfonyl)benzenesulfonamide (8) (97 mg, 0.18 mmol), DMAP (48 mg, 0.39 mmol), and N-(3-Dimethylaminopropyl)-N′-ethylcarbodiimide hydrochloride (41 mg, 0.22 mmol) sequentially. The resulting mixture was allowed to stir at room temperature overnight and then condensed under reduced pressure to afford a residue which was chromatographed on silical gel (20:1 DCM:MeOH) to yield the title compound (127 mg, 62% yield) as a yellowish solid. ¹H NMR (600 MHz, CDCl₃) δ 8.36 (d, J=2.2 Hz, 1H), 8.11 (dd, J=9.2, 2.1 Hz, 1H), 7.66 (d, J=9.0 Hz, 2H), 7.37 (dd, J=5.2, 3.4 Hz, 2H), 7.34-7.26 (m, 5H), 7.26-7.24 (m, 1H), 7.07 (d, J=8.6 Hz, 1H), 7.03-6.98 (m, 2H), 6.78 (d, J=8.8 Hz, 2H), 6.61 (d, J=9.5 Hz, 1H), 4.06 (s, 2H), 3.96-3.87 (m, 1H), 3.70-3.64 (m, 5H), 3.64-3.57 (m, 3H), 3.49-3.42 (m, 1H), 3.26 (s, 4H), 3.10 (dd, J=13.9, 5.1 Hz, 1H), 3.02 (dd, J=13.9, 7.2 Hz, 1H), 2.89 (s, 2H), 2.50-2.42 (m, 2H), 2.41-2.30 (m, 10H), 2.12 (ddd, J=10.4, 5.1, 1.9 Hz, 1H), 2.10 (s, 1H), 1.68 (dq, J=8.1, 5.6 Hz, 1H), 1.49 (s, 9H) ppm. ESI m/z 1047.2 (M+H)⁺.

### General Preparation of 15-18:

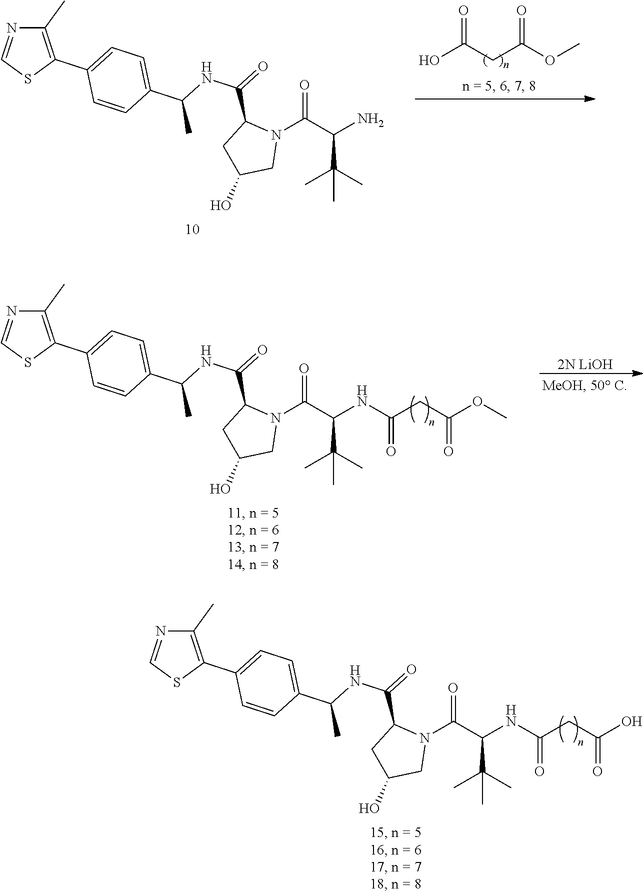

To a solution of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide (10) (1.0 eq) in DCM was added an corresponding carboxylic acid (1.1 eq), triethyl amine, and HATU (1.1 eq) sequentially. After stirring at room temperature overnight, the mixture was condensed and chromatographed on silical gel to afford the compounds 11-14.

To a solution of 11-14 in methanol was added 2N LiOH aqueous (Methol/LiOH_{aq}=2/1 v/v). After stirring at 50° C. for 2 h, the mixture was cooled down and the pH was adjusted to 7 with 3N HCl. The mixture was then extracted with methylene chloride (3×) and the combined organic layers were washed with brine, dried over sodium sulfate, and condensed to afford compounds 15-18, which were used to next step without purification.

Compound 15: ¹H NMR (400 MHz, CDCl₃ and CD₃OD) δ 8.72 (s, 1H), 8.05-7.89 (m, 1H), 7.43-7.33 (m, 4H), 7.24-7.08 (m, 1H), 5.14-4.95 (m, 1H), 4.73-4.40 (m, 3H), 4.00-3.93 (m, 1H), 3.76-3.59 (m, 1H), 2.52 (s, 3H), 2.38-2.05 (m, 6H), 1.71-1.49 (m, 9H), 1.04 (s, 9H).

Compound 16: ¹H NMR (400 MHz, CDCl₃) δ 8.72 (s, 1H), 7.59 (d, J=7.8 Hz, 1H), 7.40-7.33 (m, 4H), 6.92 (d, J=8.7 Hz, 1H), 5.15-4.98 (m, 1H), 4.76-4.67 (m, 1H), 4.62 (d, J=8.9 Hz, 1H), 4.52 (s, 1H), 4.04 (d, J=11.2 Hz, 1H), 3.74-3.59 (m, 1H), 2.51 (s, 3H), 2.39-2.10 (m, 6H), 1.66-1.45 (m, 7H), 1.35-1.27 (m, 4H), 1.03 (s, 9H).

### General Preparation of 19-21:

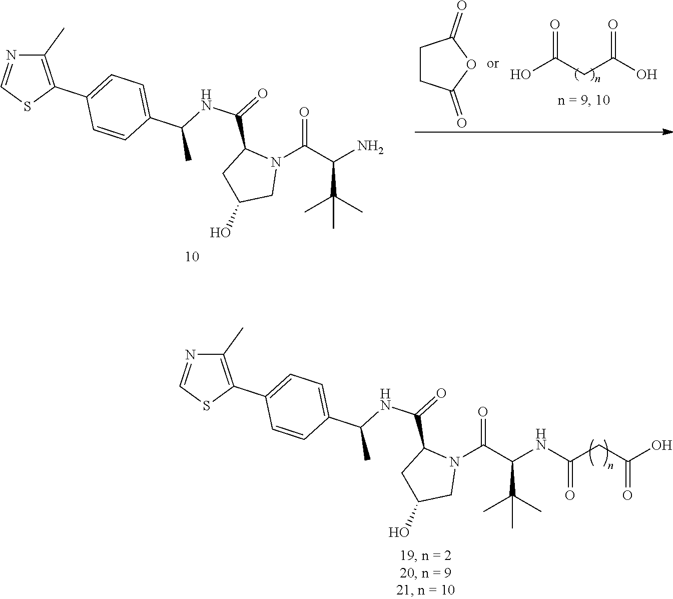

To a solution of compound 10 (1 eq) in DCM was added triethyl amine, carboxylic acid (5 eq), and HATU (1.1 eq) sequentially. After stirring at room temperature overnight, the mixture was condensed and chromatographed on silical gel to afford the compounds 19-21.

Compound 19: ¹H NMR (400 MHz, CDCl₃) δ 8.67 (s, 1H), 7.94 (d, J=8.2 Hz, 1H), 7.79 (d, J=7.7 Hz, 1H), 7.44-7.34 (m, 4H), 5.13-5.03 (m, 1H), 4.81-4.73 (m, 1H), 4.51-4.38 (m, 2H), 4.15 (d, J=11.4 Hz, 1H), 3.54 (dd, J=11.4, 3.5 Hz, 1H), 2.64-2.37 (m, 8H), 2.16-2.06 (m, 1H), 1.47 (d, J=6.9 Hz, 3H), 1.05 (s, 9H).

Compound 21: ¹H NMR (600 MHz, CDCl₃) δ 8.71 (s, 1H), 7.42 (d, J=8.2 Hz, 2H), 7.39 (d, J=8.2 Hz, 2H), 7.30-7.28 (m, 1H), 7.04 (d, J=9.1 Hz, 1H), 5.14-5.08 (m, 1H), 4.69 (dd, J=17.2, 8.7 Hz, 2H), 4.54 (s, 1H), 4.16 (d, J=11.5 Hz, 1H), 3.66 (dd, J=11.3, 3.5 Hz, 1H), 2.48 (s, 3H), 2.46 (ddd, J=12.8, 7.9, 4.5 Hz, 1H), 2.38-2.32 (m, 2H), 2.23 (dt, J=8.5, 6.3 Hz, 2H), 2.12 (dd, J=13.4, 8.0 Hz, 1H), 1.68-1.55 (m, 4H), 1.50 (d, J=6.9 Hz, 3H), 1.40-1.24 (m, 13H), 1.05 (s, 9H). ESI+, m/z 657 [M+H]⁺.

### General Preparation of 12-28:

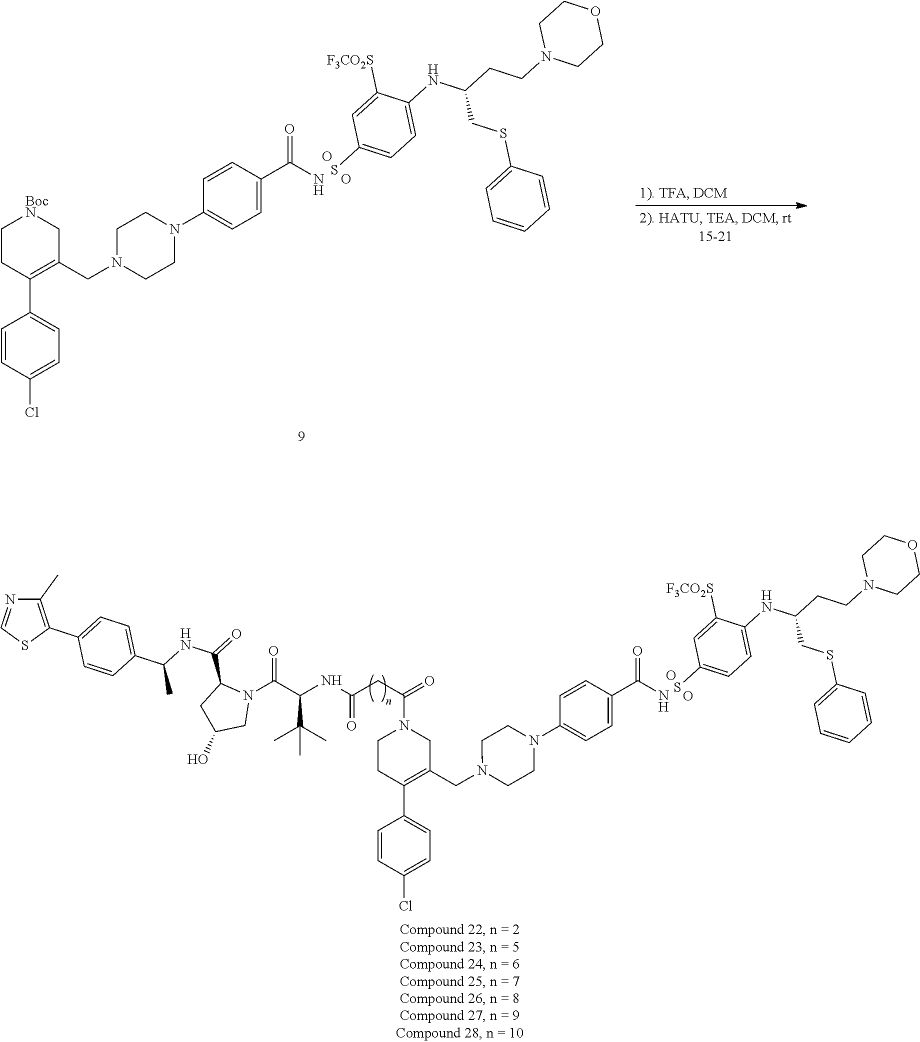

To a solution of compound 9 (1 eq) in DCM was added TFA (10 eq), after stirring at room temperature for 30 min, the resulting mixture was condensed to remove TFA. The residue was redissolved in DCM and treated with triethyl amine (3 eq), carboxylic acid compound (15, 16, 17, 18, 19, 20, or 21), and HATU (1.1 eq) at room temperature overnight. The reaction mixture was then condensed and chromatographed on silical gel to afford compounds 22-28.

### Example 1: (2S,4R)-1-((S)-2-(4-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-4-oxobutanamido)-3,3-dimethylbutanoyl)-4-hydroxy-N—((S)-1-(4-(4-methylthiazol-5-yl)phenyl)ethyl)pyrrolidine-2-carboxamide (22)

Compound 22 was obtained from compound 9 and compound 15. ¹H NMR (600 MHz, CDCl₃) δ 8.71 (d, J=10.1 Hz, 1H), 8.33 (d, J=1.9 Hz, 1H), 8.15 (ddd, J=9.2, 4.2, 2.2 Hz, 1H), 7.95 (d, J=29.5 Hz, 1H), 7.67 (dd, J=18.5, 8.9 Hz, 2H), 7.43-7.37 (m, 6H), 7.32 (dd, J=7.9, 6.9 Hz, 2H), 7.28-7.21 (m, 3H), 7.06 (d, J=8.6 Hz, 1H), 6.89 (d, J=8.0 Hz, 1H), 6.86-6.77 (m, 3.5H), 6.69 (d, J=8.6 Hz, 0.5H), 6.63 (dd, J=9.5, 2.5 Hz, 1H), 5.12 (dd, J=13.4, 6.9 Hz, 1H), 4.76 (dt, J=28.4, 8.3 Hz, 1H), 4.62 (d, J=9.0 Hz, 0.5H), 4.46 (s, 0.5H), 4.42-4.36 (m, 1H), 4.25-4.19 (d, J=17.7 Hz, 0.5H), 4.10 (d, J=17.6 Hz, 0.5H), 4.06-3.99 (m, 1H), 3.92 (d, J=10.9 Hz, 2H), 3.88-3.76 (m, 1H), 3.68 (d, J=2.1 Hz, 4H), 3.59-3.53 (m, 1.5H), 3.33-3.18 (m, 4.5H), 3.12 (dd, J=14.0, 5.1 Hz, 1H), 3.04 (ddd, J=13.8, 7.1, 4.1 Hz, 1.5H), 2.95-2.79 (m, 3H), 2.79-2.57 (m, 3H), 2.51 (dd, J=8.9, 4.4 Hz, 3H), 2.48-2.27 (m, 12H), 2.18-2.10 (m, 2H), 2.03-1.95 (m, 1H), 1.70 (dd, J=13.6, 7.0 Hz, 1.5H), 1.52 (dd, J=6.8, 4.6 Hz, 3H), 1.07 (d, J=11.6 Hz, 9H). ESI⁺, m/z 1474.4 [M+H]⁺.

### Example 2: (2S,4R)-1-((S)-2-(7-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (23)

Compound 23 was obtained from compound 9 and compound 16. ¹H NMR (600 MHz, CDCl₃) δ 8.71 (t, J=7.6 Hz, 1H), 8.38-8.34 (m, 1H), 8.15 (d, J=9.3 Hz, 1H), 7.67 (d, J=8.3 Hz, 2H), 7.53-7.37 (m, 8H), 7.36-7.31 (m, 4H), 7.08 (d, J=8.6 Hz, 1H), 7.04-7.00 (m, 2H), 6.78 (dd, J=16.8, 9.1 Hz, 2H), 6.65 (t, J=9.7 Hz, 1H), 6.27 (dd, J=18.1, 9.8 Hz, 1H), 5.16-5.06 (m, 1H), 4.82-4.73 (m, 1H), 4.65-4.59 (m, 1H), 4.53 (d, J=21.7 Hz, 1H), 4.26 (s, 1H), 4.19-4.10 (m, 2H), 3.94 (s, 1H), 3.83-3.75 (m, 1H), 3.73-3.55 (m, 6H), 3.26 (s, 4H), 3.13 (dd, J=13.9, 5.0 Hz, 1H), 3.09-3.02 (m, 1H), 2.93 (d, J=11.0 Hz, 2H), 2.56-2.48 (m, 5H), 2.38 (ddd, J=24.8, 13.2, 7.2 Hz, 11H), 2.23-2.03 (m, 5H), 1.76-1.69 (m, 2H), 1.67-1.57 (m, 5H), 1.52-1.47 (m, 3H), 1.38 (ddd, J=21.8, 14.7, 7.4 Hz, 2H), 1.06 (s, 9H). ESI⁺, m/z 1515.4 [M+H]⁺.

### Example 3: (2S,4R)-1-((S)-2-(8-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (24)

Compound 24 was obtained from compound 9 and compound 17. ¹H NMR (600 MHz, CDCl₃) δ 8.71 (t, J=8.1 Hz, 1H), 8.36 (dd, J=4.6, 2.1 Hz, 1H), 8.15 (dd, J=9.2, 2.1 Hz, 1H), 7.71 (dd, J=53.3, 8.9 Hz, 2H), 7.55-7.36 (m, 7H), 7.32 (dd, J=12.0, 5.7 Hz, 4H), 7.12-6.94 (m, 3H), 6.79 (dd, J=34.6, 9.0 Hz, 2H), 6.64 (t, J=9.9 Hz, 1H), 6.28 (d, J=8.9 Hz, 1H), 5.17-5.05 (m, 1H), 4.81-4.46 (m, 3H), 4.32-4.10 (m, 3H), 3.97-3.87 (m, 1H), 3.86-3.72 (m, 2H), 3.71-3.53 (m, 6H), 3.24 (s, 4H), 3.13 (dd, J=13.9, 5.1 Hz, 1H), 3.05 (ddd, J=13.7, 7.1, 4.5 Hz, 1H), 2.96-2.89 (m, 2H), 2.55-2.50 (m, 3H), 2.46 (s, 2H), 2.45-2.30 (m, 12H), 2.16-2.08 (m, 2H), 2.07 (t, J=7.7 Hz, 1H), 1.72-1.62 (m, 4H), 1.57-1.46 (m, 4H), 1.40-1.29 (m, 5H), 1.07 (d, J=2.5 Hz, 9H). ESI⁺, m/z 1529.8 [M+H]⁺.

### Example 4: (2S,4R)-1-((S)-2-(9-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide (25)

Compound 25 was obtained from compound 9 and compound 18. ¹H NMR (600 MHz, CDCl₃) δ 8.71 (d, J=9.9 Hz, 1H), 8.41-8.26 (m, 1H), 8.14 (t, J=9.8 Hz, 1H), 7.71 (dd, J=84.6, 8.9 Hz, 2H), 7.61-7.37 (m, 7H), 7.36-7.30 (m, 4H), 7.09 (dd, J=15.0, 6.9 Hz, 1H), 7.03 (t, J=8.5 Hz, 2H), 6.79 (dd, J=31.8, 9.0 Hz, 2H), 6.64 (t, J=9.1 Hz, 1H), 6.26 (dd, J=42.7, 8.5 Hz, 1H), 5.18-5.07 (m, 1H), 4.83-4.44 (m, 3H), 4.33-4.10 (m, 3H), 3.87 (dd, J=29.7, 22.3 Hz, 2H), 3.78-3.47 (m, 7H), 3.24 (d, J=4.3 Hz, 4H), 3.13 (dd, J=13.9, 5.0 Hz, 1H), 3.05 (dd, J=13.8, 7.1 Hz, 1H), 2.93 (d, J=10.6 Hz, 2H), 2.54 (s, 3H), 2.46 (s, 2H), 2.39 (ddd, J=20.9, 11.9, 6.4 Hz, 14H), 2.17-2.10 (m, 2H), 2.06-1.98 (m, 1H), 1.72-1.63 (m, 4H), 1.51 (dd, J=12.9, 6.9 Hz, 3H), 1.48-1.43 (m, 1H), 1.39-1.31 (m, 5H), 1.07 (d, J=7.9 Hz, 9H). ESI⁺, m/z 1544.8 [M+H]⁺.

### Example 5: (2S,4R)-1-((S)-2-(10-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-10-oxodecanamido)-3,3-dimethylbutanoyl)-4-hydroxy-N—((S)-1-(4-(4-methylthiazol-5-yl)phenyl)ethyl)pyrrolidine-2-carboxamide (26)

Compound 26 was obtained from compound 9 and compound 19. ¹H NMR (600 MHz, CDCl₃) δ 8.70 (d, J=6.2 Hz, 1H), 8.39-8.25 (m, 1H), 8.13 (t, J=10.4 Hz, 1H), 7.74 (dd, J=94.6, 8.9 Hz, 2H), 7.53-7.37 (m, 6H), 7.33 (dt, J=9.9, 8.2 Hz, 5H), 7.08 (t, J=8.6 Hz, 1H), 7.03 (t, J=7.7 Hz, 2H), 6.78 (dd, J=19.5, 9.1 Hz, 2H), 6.64 (t, J=8.9 Hz, 1H), 6.36-6.16 (m, 1H), 5.12 (dd, J=14.9, 7.6 Hz, 1H), 4.86-4.47 (m, 3H), 4.32-4.19 (m, 2H), 4.14 (dt, J=24.4, 10.4 Hz, 1H), 3.97 (dt, J=22.9, 8.4 Hz, 2H), 3.74-3.58 (m, 7H), 3.29-3.19 (m, 4H), 3.12 (dd, J=13.8, 5.1 Hz, 1H), 3.05 (dd, J=13.8, 7.1 Hz, 1H), 2.94 (s, 2H), 2.54 (s, 3H), 2.48-2.29 (m, 14H), 2.12 (dd, J=19.6, 12.7 Hz, 2H), 1.75-1.58 (m, 6H), 1.50 (dd, J=14.3, 6.9 Hz, 3H), 1.43 (d, J=15.1 Hz, 2H), 1.38-1.29 (m, 5H), 1.20-1.15 (m, 2H), 1.07 (d, J=6.1 Hz, 9H). ESI⁺, m/z 1557.6 [M+H]⁺.

### Example 6: (2S,4R)-1-((S)-2-(11-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-11-oxoundecanamido)-3,3-dimethylbutanoyl)-4-hydroxy-N—((S)-1-(4-(4-methylthiazol-5-yl)phenyl)ethyl)pyrrolidine-2-carboxamide (27)

Compound 27 was obtained from compound 9 and compound 20. ¹H NMR (600 MHz, CDCl₃) δ 8.68 (s, 1H), 8.31 (dd, J=33.0, 1.8 Hz, 1H), 8.16-8.06 (m, 1H), 7.73 (dd, J=74.2, 8.9 Hz, 2H), 7.46-7.35 (m, 7H), 7.35-7.28 (m, 5H), 7.25 (t, J=4.7 Hz, 1H), 7.08-6.96 (m, 3H), 6.76 (dd, J=15.5, 9.0 Hz, 2H), 6.62 (t, J=9.7 Hz, 1H), 6.27 (dd, J=76.0, 8.7 Hz, 1H), 5.13-5.06 (m, 1H), 4.82-4.48 (m, 3H), 4.17 (ddd, J=33.1, 22.7, 13.3 Hz, 3H), 3.91 (s, 1H), 3.83-3.71 (m, 2H), 3.70-3.56 (m, 6H), 3.22 (s, 4H), 3.10 (dd, J=13.9, 5.1 Hz, 1H), 3.03 (dd, J=13.8, 7.1 Hz, 1H), 2.91 (s, 2H), 2.51 (d, J=6.8 Hz, 3H), 2.46-2.27 (m, 14H), 2.14-2.03 (m, 4H), 1.73-1.60 (m, 4H), 1.48 (dd, J=10.4, 7.1 Hz, 3H), 1.34 (dt, J=22.5, 7.4 Hz, 4H), 1.27 (s, 2H), 1.19 (d, J=7.0 Hz, 2H), 1.16-1.07 (m, 3H), 1.05 (d, J=9.7 Hz, 9H). ESI⁺, m/z 1571 [M+H]⁺.

### Example 7: (2S,4R)-1-((S)-2-(12-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-12-oxododecanamido)-3,3-dimethylbutanoyl)-4-hydroxy-N—((S)-1-(4-(4-methylthiazol-5-yl)phenyl)ethyl)pyrrolidine-2-carboxamide (28)

Compound 28 was obtained from compound 9 and compound 21. ¹H NMR (600 MHz, CDCl₃) δ 8.69 (s, 1H), 8.33 (dd, J=31.9, 1.8 Hz, 1H), 8.17-8.08 (m, 1H), 7.76 (dd, J=63.9, 8.9 Hz, 2H), 7.46-7.36 (m, 7H), 7.35-7.29 (m, 5H), 7.10-6.94 (m, 3H), 6.77 (t, J=8.2 Hz, 2H), 6.64 (t, J=9.9 Hz, 1H), 6.45-6.19 (m, 1H), 5.14-5.05 (m, 1H), 4.82-4.68 (m, 2H), 4.54 (s, 1H), 4.23 (dd, J=34.1, 17.2 Hz, 2H), 4.14 (dd, J=11.9, 4.6 Hz, 1H), 3.98-3.86 (m, 2H), 3.66 (ddd, J=16.1, 14.1, 11.1 Hz, 7H), 3.25 (s, 4H), 3.12 (dd, J=13.8, 5.0 Hz, 1H), 3.04 (dd, J=13.8, 7.1 Hz, 1H), 2.93 (s, 2H), 2.54 (d, J=4.1 Hz, 3H), 2.38 (td, J=27.5, 14.1 Hz, 14H), 2.17-2.07 (m, 4H), 1.75-1.56 (m, 5H), 1.49 (d, J=6.9 Hz, 3H), 1.46 (d, J=6.2 Hz, 2H), 1.39-1.30 (m, 4H), 1.24-1.19 (m, 2H), 1.18-1.10 (m, 4H), 1.07 (d, J=9.1 Hz, 9H). ESI⁺, m/z 1585 [M+H]⁺.

### General Preparation of 31-32:

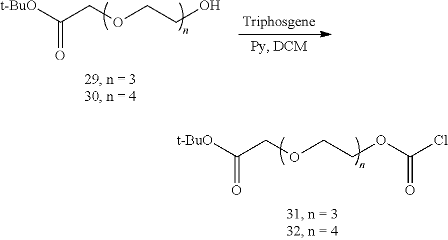

To a solution of alcohol compound 28 or 30 (1 eq) in DCM was added triphosgene (0.5 eq) and pyridine (1 eq) at 0° C., the resulting mixture was warm to room temperature and stirred for 2 h. The mixture was then diluted with ethyl acetate and washed with aqueous HCl solution, brine, and dried over sodium sulfate. Condensation of the mixture give the corresponding product 31 and 32, respectively, as a residue which was used in the next step without further purification.

### General Preparation of 33-34:

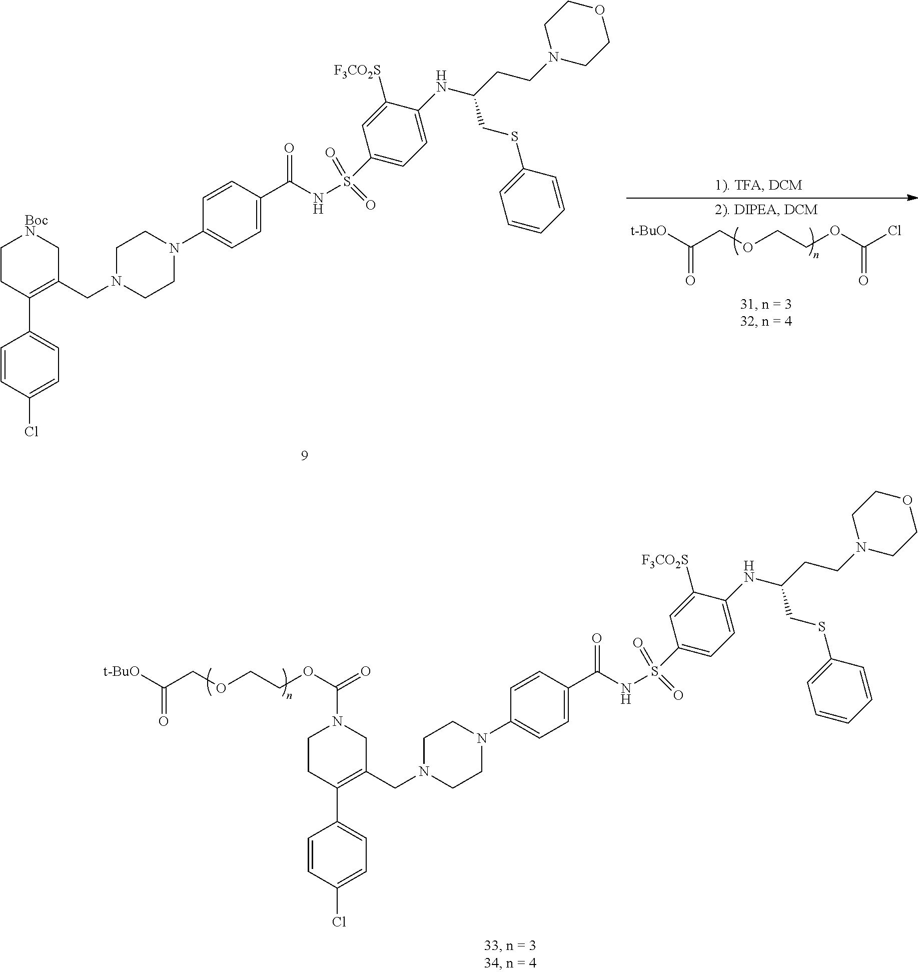

To a solution of chloride compound 31 or 32 (2 eq) in DCM was added compound 9 (1 eq) and DIPEA (6 eq). The resulting mixture was stirred at room temperature overnight, and condensed to give a residue which was chromatographed on silical gel to afford the corresponding product 33 and 34, respectively.

Compound 33. ¹H NMR (600 MHz, CDCl₃) δ 8.35 (s, 1H), 8.12 (d, J=8.8 Hz, 1H), 7.67 (d, J=24.4 Hz, 2H), 7.37 (d, J=7.4 Hz, 2H), 7.33-7.27 (m, 5H), 7.10-I 7.03 (m, 1H), 7.01 (d, J=8.4 Hz, 2H), 6.79 (s, 2H), 6.61 (d, J=9.3 Hz, 1H), 4.34-4.19 (m, 2H), 4.12 (s, 2H), 4.01 (s, 2H), 3.90 (s, 1H), 3.75-3.72 (m, 2H), 3.66 (dd, J=14.8, 9.6 Hz, 12H), 3.57 (s, 2H), 3.26 (s, 4H), 3.10 (dd, J=13.9, 5.0 Hz, 1H), 3.02 (dd, J=13.9, 7.2 Hz, 1H), 2.90 (s, 2H), 2.49-2.24 (m, 12H), 2.19-2.06 (m, 1H), 1.75-1.62 (m, 1H), 1.47 (s,

Compound 34. ¹H NMR (600 MHz, CDCl₃) δ 8.33 (d, J=1.6 Hz, 1H), 8.00 (dd, J=12.0, 5.1 Hz, 1H), 7.86 (s, 2H), 7.38 (dd, J=6.3, 5.0 Hz, 2H), 7.32-7.27 (m, 5H), 7.04-6.98 (m, 2H), 6.86 (s, 1H), 6.77 (d, J=8.0 Hz, 2H), 6.51 (dd, J=9.3, 5.7 Hz, 1H), 4.31-4.26 (m, 2H), 4.12 (s, 2H), 4.02 (s, 2H), 3.88-3.80 (m, 1H), 3.76-3.72 (m, 2H), 3.70-3.62 (m, 14H), 3.62-3.59 (m, 4H), 3.22 (s, 4H), 3.08 (dd, J=13.8, 4.8 Hz, 1H), 2.95 (dd, J=13.8, 7.8 Hz, 1H), 2.89 (s, 2H), 2.41 (s, 2H), 2.40-2.30 (m, 10H), 2.16-2.07 (m, 2H), 1.65-1.61 (m, 1H), 1.47 (s, 9H); ESI m/z 1281.3 [M+H]⁺.

### General Preparation of 35-36:

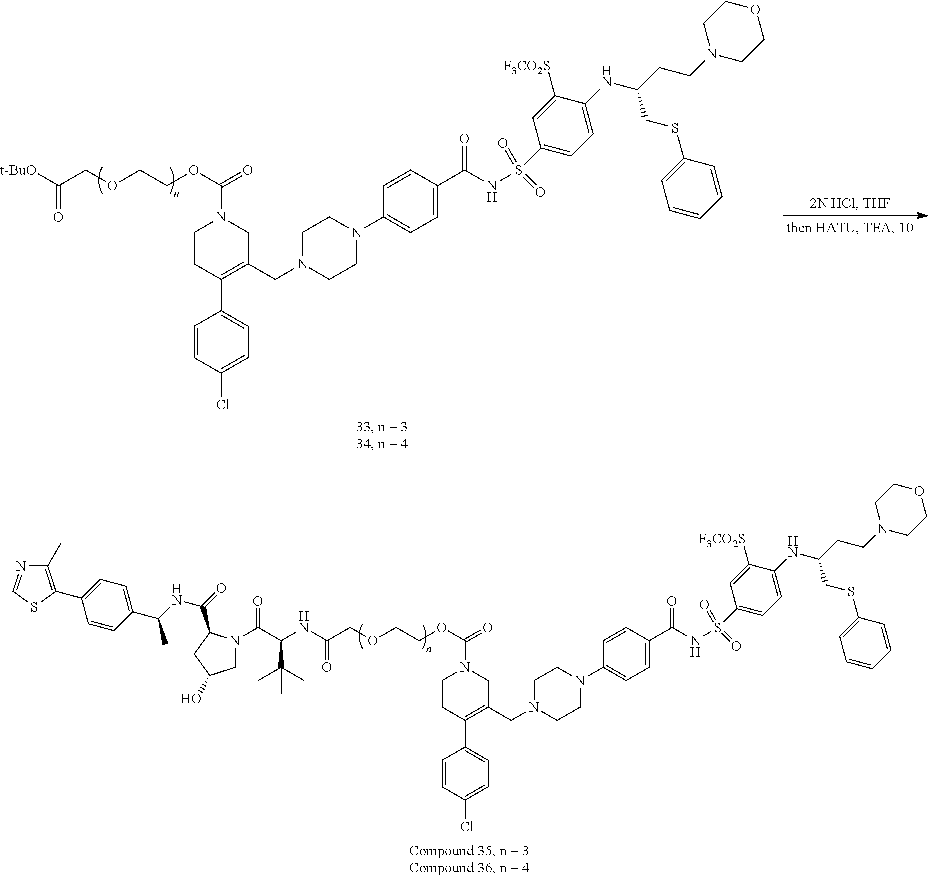

To a solution of compound 33 or 34 (1 eq) in THF was added 4 N HCl solution (in 1,4-dioxane). The resulting mixture was stirred at room temperature for 2 h and then condensed under reduced pressure to give a residue which was then treated with triethylamine (3 eq), compound 10 (1 eq) and HATU (1.1 eq) in DCM for overnight to afford Compounds 35 and 36, respectively.

### Example 8: (S)-13-((2S,4R)-4-hydroxy-2-(((S)-1-(4-(4-methylthiazol-5-yl)phenyl)ethyl)carbamoyl)pyrrolidine-1-carbonyl)-14,14-dimethyl-11-oxo-3,6,9-trioxa-12-azapentadecyl 4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridine-1(2H)-carboxylate (35)

¹H NMR (600 MHz, CDCl₃) δ 8.71 (s, 1H), 8.36 (s, 1H), 8.14 (d, J=9.0 Hz, 1H), 7.77-7.70 (m, 2H), 7.58-7.52 (m, 1H), 7.40 (ddd, J=9.1, 8.2, 6.4 Hz, 6H), 7.33 (dd, J=9.8, 4.6 Hz, 5H), 7.29 (d, J=1.2 Hz, 1H), 7.08 (d, J=8.5 Hz, 1H), 7.05-7.01 (m, 2H), 6.80 (d, J=7.3 Hz, 2H), 6.65 (d, J=8.9 Hz, 1H), 5.13 (dd, J=13.2, 6.0 Hz, 1H), 4.79 (s, 1H), 4.67 (s, 1H), 4.56 (s, 1H), 4.33 (dt, J=8.9, 5.8 Hz, 3H), 4.27-4.17 (m, 1H), 4.15 (s, 2H), 4.01 (p, J=5.0 Hz, 2H), 3.93 (s, 1H), 3.74 (d, J=4.2 Hz, 2H), 3.72-3.64 (m, 10H), 3.62-3.53 (m, 2H), 3.31-3.22 (m, 4H), 3.13 (dd, J=13.9, 5.1 Hz, 1H), 3.05 (dd, J=13.9, 7.1 Hz, 1H), 2.93 (s, 2H), 2.52 (s, 3H), 2.45 (s, 2H), 2.43-2.33 (m, 10H), 2.18-2.09 (m, 2H), 1.51 (d, J=6.9 Hz, 3H), 1.39-1.35 (m, 4H), 1.09 (s, 9H). ESI⁺, m/z 1607.5 [M+H]⁺.

### Example 9: (S)-16-((2S,4R)-4-hydroxy-2-(((S)-1-(4-(4-methylthiazol-5-yl)phenyl)ethyl)carbamoyl)pyrrolidine-1-carbonyl)-17,17-dimethyl-14-oxo-3,6,9,12-tetraoxa-15-azaoctadecyl 4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridine-1(2H)-carboxylate (36)

¹H NMR (600 MHz, CDCl₃) δ 8.70 (s, 1H), 8.35 (s, 1H), 8.13 (dd, J=9.2, 2.0 Hz, 1H), 7.76 (s, 1H), 7.73-7.64 (m, 1H), 7.40 (dt, J=17.9, 8.4 Hz, 6H), 7.36-7.31 (m, 6H), 7.06 (d, J=8.6 Hz, 1H), 7.03 (d, J=8.4 Hz, 2H), 6.79 (d, J=8.5 Hz, 2H), 6.63 (d, J=9.3 Hz, 1H), 5.16-5.06 (m, 1H), 4.77 (t, J=7.9 Hz, 1H), 4.66 (s, 1H), 4.54 (s, 1H), 4.36-4.28 (m, 2H), 4.16 (d, J=18.6 Hz, 3H), 3.97 (s, 1H), 3.92 (dd, J=7.7, 4.2 Hz, 2H), 3.75 (dd, J=8.3, 3.5 Hz, 2H), 3.68 (dd, J=10.6, 5.1 Hz, 12H), 3.59 (s, 4H), 3.27 (d, J=4.7 Hz, 4H), 3.12 (dd, J=13.9, 5.0 Hz, 1H), 3.04 (dd, J=13.9, 7.3 Hz, 1H), 2.92 (s, 2H), 2.53 (s, 3H), 2.44 (s, 2H), 2.37 (dd, J=12.0, 6.3 Hz, 8H), 2.34-2.29 (m, 2H), 2.12 (dd, J=18.1, 12.5 Hz, 2H), 1.74-1.64 (m, 4H), 1.49 (d, J=6.9 Hz, 3H), 1.35 (t, J=7.3 Hz, 2H), 1.09 (s, 9H). ESI⁺, m/z 1651 [M+H]⁺.

### Preparation of 38:

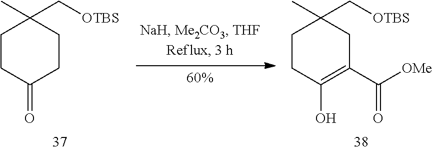 [SMILES: CC1(CO[Si](C)(C)C(C)(C)C)CCC(=O)CC1 ; COC(=O)C1=C(O)CCC(C)(CO[Si](C)(C)C(C)(C)C)C1]

To a solution of compound 37 (7.05 g, 27.54 mmol) in THF (90 mL) at 0° C. was added NaH (3.3 g, 82.62 mmol) portion wise and the mixture was stirred at the same temperature for 1 h. Neat Me₂CO₃ (7.4 g, 82.62 mmol) was added to the mixture and the solution was heated under reflux for 3 h. The reaction was quenched with saturated NH₄Cl solution at 0° C. and the THF was removed under reduced pressure. The residue was diluted with EtOAc and washed with water and brine. The organic portion was dried over anhydrous Na₂SO₄ and the solvent was removed under reduced pressure. The crude material was purified by flash chromatography (Hexanes/EtOAc=10:1) to afford 38 (5.1 g, 16.5 mmol) in 60% yield. ¹H NMR (600 MHz, Chloroform-d) δ 12.15 (s, 1H), 3.75 (s, 3H), 3.35 (d, J=9.6 Hz, 1H), 3.28 (d, J=9.5 Hz, 1H), 2.28 (ddd, J=7.4, 5.4, 1.3 Hz, 2H), 2.12 (dt, J=15.9, 1.7 Hz, 1H), 1.94-1.90 (m, 1H), 1.62 (dt, J=13.2, 7.4 Hz, 1H), 1.40 (ddt, J 13.4, 6.1, 1.3 Hz, 1H), 0.90 (s, 3H), 0.89 (s, 9H), 0.02 (d, J=1.4 Hz, 6H) ppm.

### Preparation of 39:

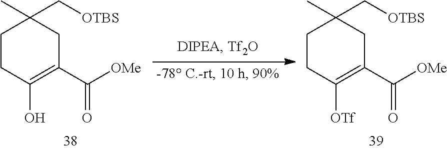 [SMILES: COC(=O)C1=C(O)CCC(C)(CO[Si](C)(C)C(C)(C)C)C1 ; COC(=O)C1=C(OS(=O)(=O)C(F)(F)F)CCC(C)(CO[Si](C)(C)C(C)(C)C)C1]

To a stirring solution of compound 38 (5.1 g, 16.5 mmol) in DCM (65 mL) was added DIPEA (14.5 mL, 82.5 mmol) at −78° C. and the mixture was stirred for 0.5 h at the same temperature. Tf₂O (4.2 ml, 24.75 mmol) was added to the reaction mixture and it was stirred for 10 h at room temperature. The reaction was diluted with DCM (100 mL) and quenched with water. The organic portion was washed with dilute HCl followed by brine solution. The organic portion was dried over anhydrous Na₂SO₄ and the solvent was removed in vacuo. The crude material was purified by flash chromatography (Hexanes/EtOAc=10:1) to get the pure triflate 39 (6.6 g, 14.8 mmol) in 90% yield. ¹H NMR (600 MHz, Chloroform-d) δ 3.80 (s, 3H), 3.37 (d, J=9.7 Hz, 1H), 3.30 (d, J=9.6 Hz, 1H), 2.41 (ddd, J=15.4, 7.2, 3.0 Hz, 3H), 2.20-2.15 (m, 1H), 1.77-1.71 (m, 1H), 1.47 (ddd, J=11.9, 8.2, 5.3 Hz, 1H), 0.93 (s, 3H), 0.89 (s, 9H), 0.03 (s, 6H) ppm.

### Preparation of 40:

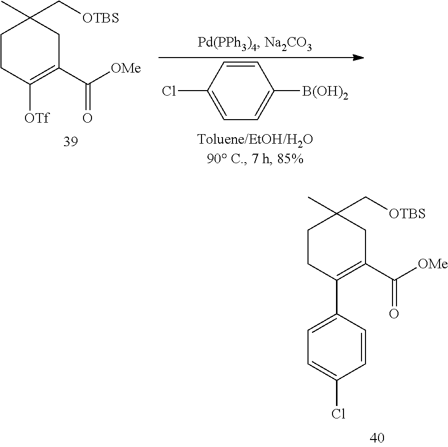 [SMILES: COC(=O)C1=C(OS(=O)(=O)C(F)(F)F)CCC(C)(CO[Si](C)(C)C(C)(C)C)C1 ; OB(O)c1ccc(Cl)cc1 ; COC(=O)C1=C(c2ccc(Cl)cc2)CCC(C)(CO[Si](C)(C)C(C)(C)C)C1]

To the solution of triflate 39 (6.6 g, 14.8 mmol) in toluene (28 mL) and EtOH (14.8 mL) was added 2N Na₂CO₃ solution (14.8 mL). The above mixture was purged with argon for 15 min and 4-chlorophenylboronic acid (3 g, 19.24 mmol) and Pd(PPh₃)₄ (170 mg, 0.148 mmol) was added to it. The mixture was heated to 90° C. and the reaction was completed in 7 h. Ethanol was removed in vacuo and the reaction was diluted with EtOAc (150 mL). The above mixture was washed with water and brine solution. The organic portion was dried over anhydrous Na₂SO₄ and the solvent was removed in vacuo. The crude material was purified by flash chromatography (Hexanes/EtOAc=10:1) to get the pure ester 40 (5.1 g, 12.58 mmol) in 85% yield. ¹H NMR (600 MHz, Chloroform-d) δ 7.28 (d, J=8.4 Hz, 2H), 7.06 (d, J=8.4 Hz, 2H), 3.45 (s, 3H), 3.40 (d, J=9.5 Hz, 1H), 3.34 (d, J=9.5 Hz, 1H), 2.38-2.30 (m, 3H), 2.14-2.09 (m, 1H), 1.67 (dt, J=13.9, 7.2 Hz, 1H), 1.43 (dtd, J=12.9, 5.6, 1.4 Hz, 1H), 0.95 (s, 3H), 0.90 (s, 9H), 0.04 (d, J=2.7 Hz, 6H) ppm.

### Preparation of 41:

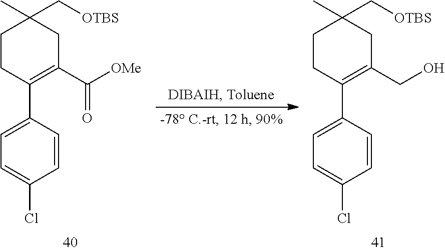 [SMILES: COC(=O)C1=C(c2ccc(Cl)cc2)CCC(C)(CO[Si](C)(C)C(C)(C)C)C1 ; CC1(CO[Si](C)(C)C(C)(C)C)CCC(c2ccc(Cl)cc2)=C(CO)C1]

To a solution of ester 40 (5.1 g, 12.58 mmol) in toluene (48 mL) was added DIBAL-H (1M in toluene, 28 mL) at −78° C. and the mixture stirred for 5 h at room temp. The reaction was diluted with 50 ml toluene and was quenched by adding saturated solution of Rochelle's salt at 0° C. drop wise. Then the reaction was filtered through celite and the filtrate was dried over anhydrous Na₂SO₄ and concentrated in vacuo. The crude material was purified by flash chromatography (Hexanes/EtOAc=3:1) to get the pure alcohol 41 (4.3 g, 11.32 mmol) in 90% yield. ¹H NMR (600 MHz, Chloroform-d) δ 7.29 (d, J=8.4 Hz, 2H), 7.08 (d, J=8.4 Hz, 2H), 3.92 (d, J=3.9 Hz, 2H), 3.42-3.32 (m, 2H), 2.31-2.23 (m, 2H), 2.21-2.15 (m, 1H), 1.95-1.89 (m, 1H), 1.63 (ddd, J=13.0, 8.0, 6.6 Hz, 1H), 1.42 (ddt, J=12.9, 5.8, 1.3 Hz, 1H), 0.95 (s, 3H), 0.91 (s, 9H), 0.05 (s, 6H) ppm.

### Preparation of 42:

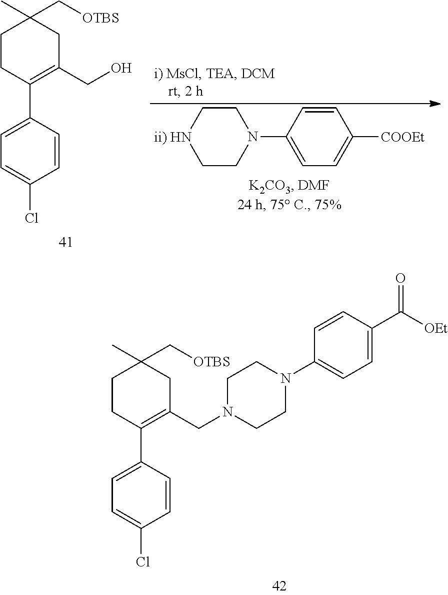

To a stirring solution of alcohol 41 (4.3 g, 11.32 mmol) DCM (55 mL) at 0° C. was added triethylamine (3.1 ml, 22.64 mmol) followed by the addition of methanesulfonyl chloride (1.3 mL, 17 mmol). The reaction was stirred for 2 h at rt and then quenched with saturated NaHCO₃. The reaction was diluted with 50 ml DCM and the organic part was washed with water followed by brine. The organic portion was dried over anhydrous Na₂SO₄ and the solvent was removed in vacuo to get the crude product which was used in the next step without further purification.

The crude mesylate from above was dissolved in DMF (25 mL) followed by the addition of K₂CO₃ (3.1 g, 22.64 mmol) and ethyl 4-(piperazin-1-yl)benzoate (3.4 g, 14.71 mmol). The mixture was stirred at 75° C. for 24 h. Upon consumption of the starting material (monitored by TLC), the mixture was allowed to come to the room temperature and diluted with 150 mL EtOAc and successively washed with water (25 mL×3) and brine solution. The organic portion was dried over anhydrous Na₂SO₄ and the solvent was removed in vacuo. The crude material was purified by flash chromatography (Hexanes/EtOAc=2:1) to afford the title compound 42 (5.0 g g, 8.49 mmol) in 75% yield in two steps. ¹H NMR (600 MHz, Chloroform-d) δ 7.90 (d, J=9.0 Hz, 2H), 7.27 (d, J=8.4 Hz, 2H), 7.00 (d, J=8.4 Hz, 2H), 6.81 (d, J=9.0 Hz, 2H), 4.32 (q, J=7.1 Hz, 2H), 3.41-3.33 (m, 2H), 3.25 (t, J=5.1 Hz, 4H), 2.80 (s, 2H), 2.39-2.32 (m, 4H), 2.28-2.19 (m, 2H), 2.17-2.11 (m, 1H), 1.94-1.89 (m, 1H), 1.63 (ddd, J=13.2, 8.6, 6.4 Hz, 1H), 1.46-1.40 (m, 1H), 1.36 (t, J=7.1 Hz, 3H), 0.94 (s, 3H), 0.91 (s, 9H), 0.05 (d, J=0.9 Hz, 6H) ppm.

### Preparation of 43:

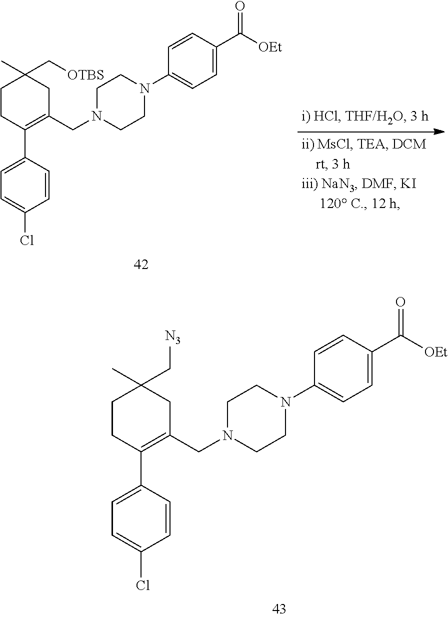 [SMILES: CCOC(=O)c1ccc(N2CCN(CC3=C(c4ccc(Cl)cc4)CCC(C)(CO[Si](C)(C)C(C)(C)C)C3)CC2)cc1 ; CCOC(=O)c1ccc(N2CCN(CC3=C(c4ccc(Cl)cc4)CCC(C)(CN=[N+]=[N-])C3)CC2)cc1]

Compound 42 (5.0 g g, 8.49 mmol) was dissolved in 40 mL THE followed by the addition of 3N HCl (10 mL) and the mixture was stirred at room temperature for 3 h. Upon consumption of the staring material (monitored by TLC) the pH was adjusted to 7 by adding saturated NaHCO₃ solution. THE was removed under reduced pressure and the mixture was mixed with EtOAc (200 mL). The organic part was washed with water and brine solution. The organic portion was dried over anhydrous Na₂SO₄ and the solvent was removed in vacuo. The crude material was in the next step without further purification.

To a stirring solution of crude alcohol in DCM (35 mL) at 0° C. was added triethylamine (2 mL, 15.28 mmol) followed by the addition of methanesulfonyl chloride (0.88 mL, 11.46 mmol). The reaction was stirred for 3 h at room temperature and then quenched with saturated NaHCO₃. The reaction was diluted with 40 mL DCM and the organic part was washed with water followed by brine. The organic portion was dried over anhydrous Na₂SO₄ and the solvent was removed in vacuo to get the crude product which was used in the next step without further purification.

To a solution of the crude mesylate from the previous reaction in DMF (14 mL) was added NaN₃ (988 mg, 15.2 mmol) and KI (cat. amt). The above mixture was heated to 120° C. for 12 h. Upon completion of the reaction the mixture was diluted with EtOAc (150 mL) and was washed successively with water (20 ml×3) and brine solution. The organic portion was dried over anhydrous Na₂SO₄ and the solvent was removed in vacuo. The crude material was purified by flash chromatography (Hexane:EtOAc=2:1) to afford 43 (3.48 g, 6.87 mmol) in 81% yield in three steps. ¹H NMR (600 MHz, Chloroform-d) δ 7.90 (d, J=9.0 Hz, 2H), 7.28 (d, J=8.4 Hz, 2H), 6.99 (d, J=8.4 Hz, 2H), 6.81 (d, J=9.0 Hz, 2H), 4.32 (q, J=7.1 Hz, 2H), 3.29-3.25 (m, 4H), 3.22 (d, J=11.8 Hz, 2H), 2.80 (s, 2H), 2.35 (t, J=5.1 Hz, 4H), 2.31-2.23 (m, 2H), 2.17-2.12 (m, 1H), 2.05-2.01 (m, 1H), 1.62 (dt, J=13.7, 6.9 Hz, 1H), 1.54-1.48 (m, 1H), 1.36 (t, J=7.1 Hz, 3H), 1.04 (s, 3H).

### Preparation of 44:

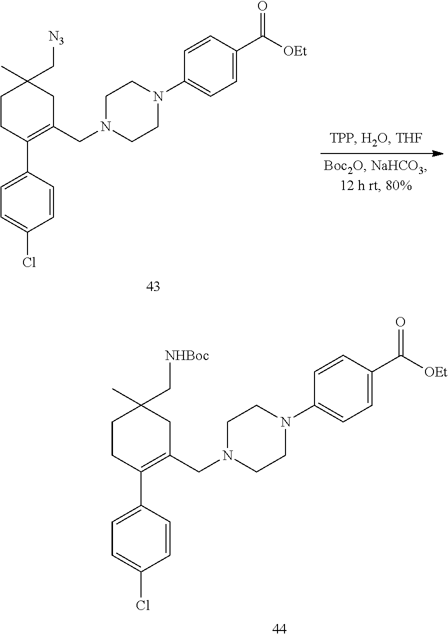 [SMILES: CCOC(=O)c1ccc(N2CCN(CC3=C(c4ccc(Cl)cc4)CCC(C)(CN=[N+]=[N-])C3)CC2)cc1 ; CCOC(=O)c1ccc(N2CCN(CC3=C(c4ccc(Cl)cc4)CCC(C)(CNC(=O)OC(C)(C)C)C3)CC2)cc1]

To a stirring solution of the azide 43 (3.48 g, 6.87 mmol) in THF (24 mL) and water (4 mL) was added triphenylphosphine (3.6 g, 13.7 mmol) and the reaction was stirred for 3 h at room temperature. To the mixture was added Boc₂O (2.2 g, 10.3 mmol) and NaHCO₃ (1.7 g, 20.6 mmol) and the reaction was stirred for 12 h. Upon completion of the reaction mixture THE was removed under reduced pressure and the reaction was diluted with EtOAc (150 ml). The organic portion was washed with water and brine solution. The organic solution was dried over anhydrous Na₂SO₄ and the solvent was removed in vacuo. The crude material was purified by flash chromatography (Hexane:EtOAc=4:1) to get the pure ester 44 (3.1 g, 5.4 mmol) in 80% yield. ¹H NMR (600 MHz, Chloroform-d) δ 7.91-7.87 (m, 2H), 7.29-7.26 (m, 2H), 7.01-6.97 (m, 2H), 6.83-6.77 (m, 2H), 4.74 (t, J=6.4 Hz, 1H), 4.31 (q, J=7.1 Hz, 2H), 3.25 (t, J=5.1 Hz, 4H), 3.13 (dd, J=13.5, 7.0 Hz, 1H), 3.05 (dd, J=13.5, 6.0 Hz, 1H), 2.79 (t, J=10.3 Hz, 2H), 2.38-2.22 (m, 6H), 2.10 (d, J=17.5 Hz, 1H), 1.98 (d, J=17.3 Hz, 2H), 1.58-1.49 (m, 2H), 1.43 (s, 9H), 1.36 (t, J=7.1 Hz, 3H), 0.97 (s, 3H).

### Preparation of 45:

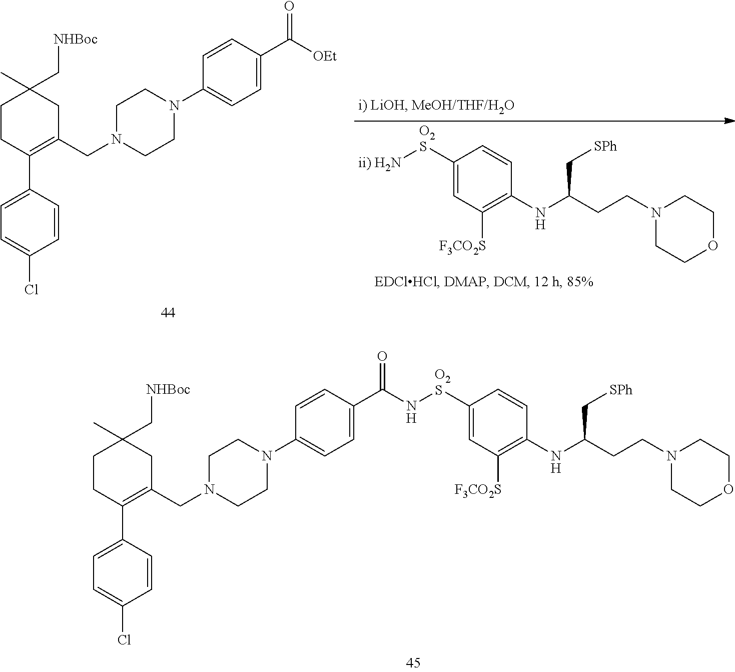

To a stirring solution of compound 44 (291 mg, 0.5 mmol) in MeOH (5 mL) and THE (1 mL) was added LiOH.H₂O (42 mg, 1 mmol) solution in H₂O (1 mL) and the mixture was stirred for 10 h at room temperature. Once the starting material was consumed the pH of the reaction was adjusted to 6 using 1N HCl. Organic solvents were removed from the mixture and crude was diluted with EtOAc (150 mL). The organic portion was washed with water and brine solution. The organic solution was dried over anhydrous Na₂SO₄ and the solvent was removed in vacuo. The crude powder was used in the next step without further purification.

To the stirring solution of the crude acid in DCM (5 mL) was added successively (R)-4-((4-morpholino-1-(phenylthio)butan-2-yl)amino)-3-((trifluoromethyl)sulfonyl)benzenesulfonamide (221 mg, 0.4 mmol), EDCI.HCl (238 mg, 1.25 mmol) and DMAP (152 mg, 1.25 mmol). The mixture was stirred at room temperature for 12 h. Once the amine was consumed, DCM was removed in vacuo and the crued was directly loaded in the column and was purified by flash chromatography (DCM/MeOH=95:7) to afford 45 (368 mg, 0.34 mmol) in 85% yield with respect to the amine. MS (ESI): [M+H]⁺=1089.1

### Preparation of 46:

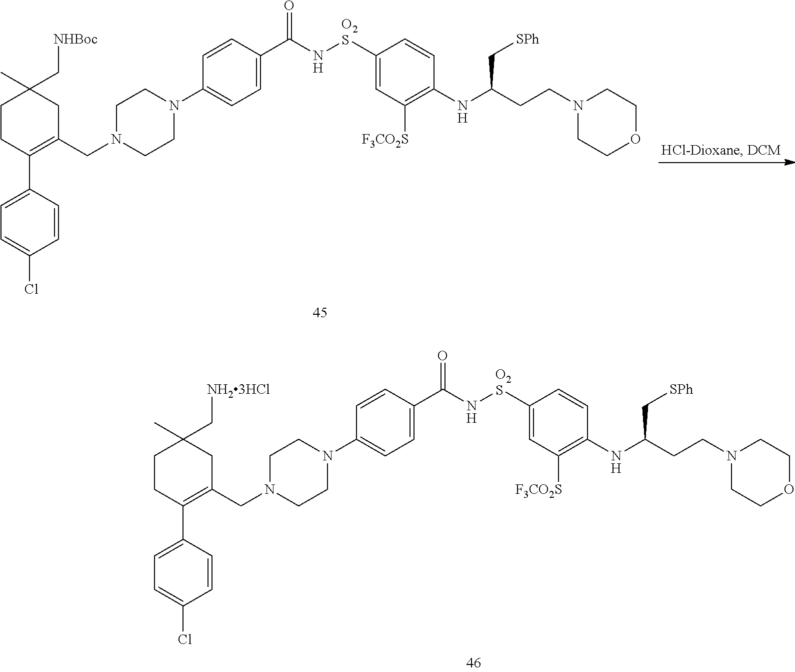

To a stirring solution of compound 45 (368 mg, 0.34 mmol) in DCM (5 mL) was added 4N HCl solution (0.34 mL, 1.36 mmol) in dioxane and the mixture was stirred at room temperature for 5 h. After consumption of the starting material the solvent was removed in vacuo and the remaining white power was washed with Et₂O (3 mL). The HCl salt of 46 was used directly without further purification. MS (ESI): [M+H]⁺=989.1.

### General Preparation of 49-56:

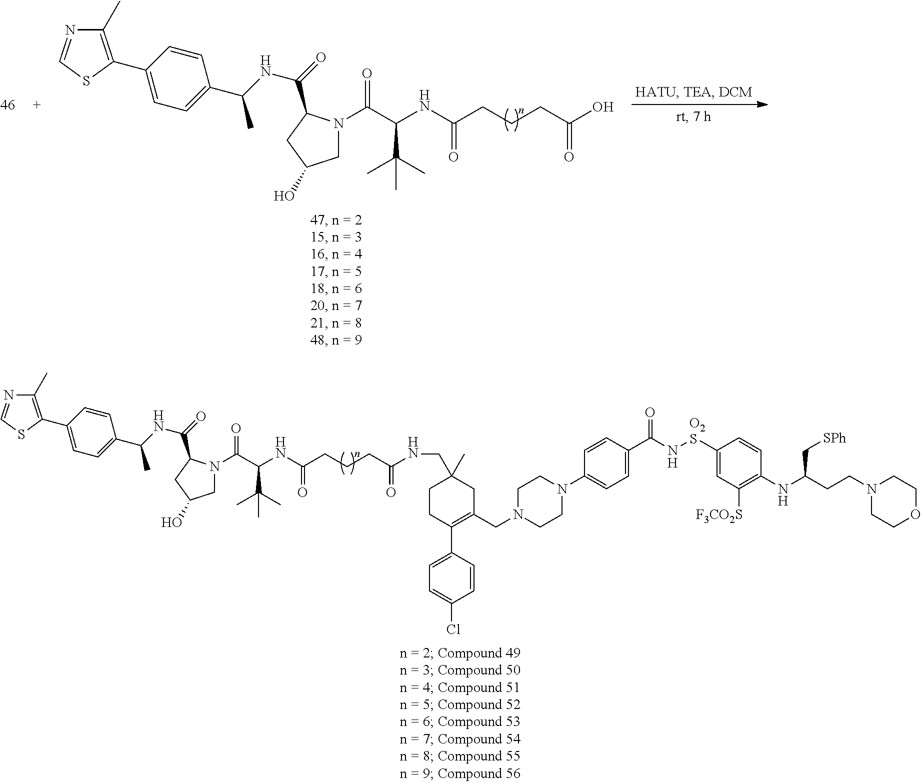

### Example 10: N′-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N⁶—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)adipamide (49)

To a stirring solution of 46 (12 mg, 0.011 mmol) and acid 47 (7 mg, 0.012 mmol) in DCM (1 mL) was added trimethylamine (0.01 mL, 0.066 mmol) and HATU (5 mg, 0.012 mmol) at room temperature. The reaction was stirred for at the same temperature for 8 h. Upon completion of the reaction the solvent was removed in vacuo and the crude product was purified by flash column chromatography (DCM:MeOH:TEA=96:3:1). After getting the product from the column the compound was mixed with 15 ml DCM and washed with sat. NH₄C1. The organic portion was dried over Na₂SO₄ and DCM was evaporated in vacuo to get Compound 49 as a pure white solid (7.71 mg, 0.005 mmol). 9.4 Hz, 1H), 7.69 (dd, J=8.7, 6.3 Hz, 2H), 7.47-7.44 (m, 1H), 7.39-7.35 (m, 6H), 7.33-7.28 (m, 4H), 7.25-7.22 (m, 1H), 7.01 (dd, J=8.3, 1.8 Hz, 2H), 6.96 (d, J=8.6 Hz, 1H), 6.62 (d, J=9.2 Hz, 3H), 5.09 (t, J=7.2 Hz, 1H), 4.74 (q, J=8.4 Hz, 1H), 4.61 (dd, J=9.0, 2.9 Hz, 1H), 4.47 (s, 1H), 4.09 (t, J=9.3 Hz, 1H), 3.90 (s, 1H), 3.65 (d, J=10.1 Hz, 5H), 3.57 (d, J=11.1 Hz, 1H), 3.35 (s, 6H), 3.11 (dd, J=13.8, 5.1 Hz, 2H), 3.03-2.99 (m, 1H), 2.49 (d, J=2.1 Hz, 3H), 2.44 (d, J=5.4 Hz, 3H), 2.35 (d, J=15.4 Hz, 7H), 2.20-2.14 (m, 3H), 2.14-2.06 (m, 5H), 2.00 (d, J=7.4 Hz, 2H), 1.67 (dd, J=14.3, 7.6 Hz, 2H), 1.54 (t, J=6.9 Hz, 5H), 1.46 (d, J=7.0 Hz, 5H), 1.29-1.23 (m, 3H), 1.03 (s, 9H), 0.99 (d, J=2.9 Hz, 3H).

### Example 11: N¹-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N⁷—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)heptanediamide (50)

Synthesized by using the same procedure used for Example 10 except acid 47 was replaced with acid 15 in 47% yield. ¹H NMR (600 MHz, Chloroform-d) δ 8.70 (d, J=1.3 Hz, 1H), 8.36 (t, J=2.5 Hz, 1H), 8.10 (ddd, J=9.3, 4.9, 2.3 Hz, 1H), 7.72 (dd, J=14.0, 8.6 Hz, 2H), 7.46 (d, J=2.8 Hz, 1H), 7.43-7.38 (m, 6H), 7.33 (dd, J=3.1, 1.7 Hz, 3H), 7.28 (s, 1H), 7.05-7.02 (m, 2H), 7.02-6.98 (m, 1H), 6.65 (d, J=9.6 Hz, 3H), 6.36 (s, 1H), 5.12 (t, J=7.2 Hz, 1H), 4.77 (dd, J=8.2, 5.6 Hz, 1H), 4.70 (d, J=8.9 Hz, 1H), 4.66 (d, J=8.8 Hz, 1H), 4.52 (s, 1H), 4.13 (q, J=8.8, 6.6 Hz, 1H), 3.93 (s, 1H), 3.71-3.65 (m, 4H), 3.62-3.59 (m, 1H), 3.31 (d, J=14.6 Hz, 6H), 3.13 (dd, J=13.8, 5.1 Hz, 2H), 3.05-3.01 (m, 1H), 2.53 (s, 3H), 2.45 (s, 4H), 2.41-2.32 (m, 7H), 2.23-2.16 (m, 4H), 2.13 (s, 4H), 2.08-2.00 (m, 3H), 1.73-1.66 (m, 2H), 1.57 (d, J=7.6 Hz, 4H), 1.50 (dd, J=6.9, 3.4 Hz, 5H), 1.36-1.30 (m, 2H), 1.24-1.19 (m, 2H), 1.06 (d, J=3.9 Hz, 9H), 1.01 (d, J=7.5 Hz, 3H).

### Example 12: N¹-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N⁸—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)octanediamide (51)

Synthesized by using the same procedure used for Example 10 except acid 47 was replaced with acid 16 in 51% yield. ¹H NMR (600 MHz, Chloroform-d) δ 8.67 (s, 1H), 8.32-8.29 (m, 1H), 8.11-8.07 (m, 1H), 7.75 (d, J=8.6 Hz, 1H), 7.72 (d, J=8.6 Hz, 1H), 7.41-7.36 (m, 6H), 7.32-7.29 (m, 3H), 7.24 (s, 1H), 7.00 (dd, J=8.4, 1.6 Hz, 4H), 6.72 (d, J=8.5 Hz, 2H), 6.62 (d, J=9.1 Hz, 1H), 6.30 (s, 1H), 6.24 (s, 1H), 5.11-5.09 (m, 1H), 4.74 (t, J=7.5 Hz, 2H), 4.69 (d, J=8.9 Hz, 1H), 4.51 (s, 1H), 4.15 (d, J=11.5 Hz, 1H), 3.90 (s, 1H), 3.65 (s, 4H), 3.58 (d, J=11.4 Hz, 1H), 3.22 (s, 6H), 3.13-3.08 (m, 2H), 3.02 (dd, J=13.9, 7.1 Hz, 2H), 2.51 (d, J=4.8 Hz, 4H), 2.42 (s, 3H), 2.35 (s, 3H), 2.30 (s, 4H), 2.14-2.08 (m, 4H), 2.04 (s, 3H), 1.63 (s, 7H), 1.48 (d, J=7.0 Hz, 4H), 1.41 (d, J=7.2 Hz, 3H), 1.19-1.11 (m, 5H), 1.05 (d, J=3.6 Hz, 9H), 0.99 (d, J=3.1 Hz, 3H).

### Example 13: N¹-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N⁹—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)nonanediamide (52)

Synthesized by using the same procedure used for Example 10 except acid 47 was replaced with acid 17 in 54% yield. ¹H NMR (600 MHz, Chloroform-d) δ 8.70 (s, 1H), 8.33 (dd, J=5.2, 2.2 Hz, 1H), 8.12-8.09 (m, 1H), 7.77 (dd, J=13.2, 8.6 Hz, 2H), 7.48-7.45 (m, 1H), 7.43-7.38 (m, 6H), 7.34-7.31 (m, 4H), 7.04-7.01 (m, 3H), 6.71 (s, 2H), 6.64 (dd, J=9.7, 3.6 Hz, 1H), 6.35 (d, J=9.5 Hz, 1H), 5.12 (td, J=7.2, 4.0 Hz, 1H), 4.78-4.73 (m, 2H), 4.53 (s, 1H), 4.15 (d, J=10.3 Hz, 1H), 3.93 (s, 2H), 3.67 (d, J=7.9 Hz, 5H), 3.62 (d, J=11.4 Hz, 1H), 3.25 (s, 6H), 3.13 (dd, J=13.8, 5.0 Hz, 2H), 3.04 (dd, J=13.9, 7.2 Hz, 2H), 2.54 (s, 3H), 2.46 (d, J=8.7 Hz, 4H), 2.35 (s, 6H), 2.19-2.11 (m, 5H), 1.70 (d, J=6.8 Hz, 5H), 1.57 (d, J=8.9 Hz, 6H), 1.53-1.48 (m, 3H), 1.45-1.39 (m, 3H), 1.24-1.19 (m, 4H), 1.13 (s, 5H), 1.07 (d, J=2.0 Hz, 9H), 1.01 (s, 3H).

### Example 14: N¹-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N¹⁰—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)decanediamide (53)

Synthesized by using the same procedure used for Example 10 except acid 47 was replaced with acid 18 in 55% yield. ¹H NMR (600 MHz, Chloroform-d) δ 8.70 (s, 1H), 8.33 (d, J=1.9 Hz, 1H), 8.11-8.09 (m, 1H), 7.77-7.73 (m, 2H), 7.43-7.37 (m, 7H), 7.34-7.31 (m, 4H), 7.04-7.00 (m, 3H), 6.70 (d, J=8.5 Hz, 2H), 6.64 (d, J=9.2 Hz, 1H), 6.32 (dd, J=14.7, 8.9 Hz, 1H), 5.11 (td, J=7.3, 3.9 Hz, 1H), 4.78-4.68 (m, 3H), 4.53 (s, 1H), 4.16 (d, J=11.5 Hz, 1H), 3.92 (s, 1H), 3.68 (q, J=5.8, 5.3 Hz, 4H), 3.64-3.60 (m, 1H), 3.32 (s, 1H), 3.25 (s, 5H), 3.13 (dd, J=13.8, 5.0 Hz, 3H), 3.04 (dd, J=13.8, 7.2 Hz, 2H), 2.54 (s, 4H), 2.45 (s, 3H), 2.35 (s, 6H), 2.22-2.17 (m, 3H), 2.17-2.10 (m, 5H), 1.69 (dd, J=14.5, 8.0 Hz, 3H), 1.58 (dt, J=22.9, 8.2 Hz, 5H), 1.50 (t, J=6.5 Hz, 4H), 1.45 (d, J=8.0 Hz, 1H), 1.23 (s, 4H), 1.15 (dd, J=14.4, 6.8 Hz, 6H), 1.08 (s, 9H), 1.01 (d, J=4.1 Hz, 3H).

### Example 15: N¹-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N¹¹—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)undecanediamide (54)

Synthesized by using the same procedure used for Example 10 except acid 47 was replaced with acid 20 in 51% yield. ¹H NMR (600 MHz, Chloroform-d) δ 8.67 (s, 1H), 8.30 (d, J=3.4 Hz, 1H), 8.10 (dd, J=9.4, 2.3 Hz, 1H), 7.74 (dd, J=15.9, 8.6 Hz, 3H), 7.41-7.35 (m, 6H), 7.30 (ddt, J=10.8, 6.3, 4.5 Hz, 6H), 7.17 (d, J=7.9 Hz, 1H), 7.00 (dd, J=8.2, 1.4 Hz, 4H), 6.71 (t, J=8.0 Hz, 2H), 6.62 (d, J=9.3 Hz, 1H), 6.38 (s, 1H), 6.31 (d, J=8.9 Hz, 1H), 5.08 (dt, J=10.8, 7.1 Hz, 2H), 4.73-4.68 (m, 3H), 4.52 (s, 1H), 4.15-4.10 (m, 2H), 3.90 (s, 2H), 3.66 (m, 5H), 3.60 (dd, J=11.5, 3.5 Hz, 2H), 3.23 (m, 7H), 3.12-3.08 (m, 2H), 3.02 (dd, J=13.9, 7.2 Hz, 2H), 2.51 (d, J=1.8 Hz, 4H), 2.42 (s, 4H), 2.30 (s, 9H), 2.22-2.16 (m, 4H), 2.13-2.07 (m, 3H), 1.48-1.39 (m, 8H), 1.16 (m, 4H), 1.05 (d, J=1.6 Hz, 9H), 0.99 (s, 3H).

### Example 16: N¹-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N²—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)dodecanediamide (55)

Synthesized by using the same procedure used for Example 10 except acid 47 was replaced with acid 21 in 50% yield with respect to amine 1. ¹H NMR (600 MHz, Chloroform-d) δ 8.67 (s, 1H), 8.31 (t, J=2.7 Hz, 1H), 8.10-8.08 (m, 1H), 7.74 (d, J=8.5 Hz, 2H), 7.40-7.33 (m, 6H), 7.32-7.26 (m, 6H), 7.04-6.98 (m, 3H), 6.69 (d, J=8.6 Hz, 2H), 6.62 (d, J=9.3 Hz, 1H), 6.33 (s, 1H), 5.11-5.05 (m, 2H), 4.70 (ddd, J=12.5, 8.4, 4.4 Hz, 3H), 4.51 (s, 1H), 4.17-4.09 (m, 2H), 3.91 (s, 1H), 3.66 (s, 4H), 3.60 (dd, J=11.5, 3.5 Hz, 2H), 3.32-3.17 (m, 7H), 3.12-3.07 (m, 2H), 3.02 (dd, J=13.9, 7.2 Hz, 2H), 2.51 (d, J=1.2 Hz, 4H), 2.43 (s, 3H), 2.34 (d, J=22.9 Hz, 6H), 2.18 (d, J=9.6 Hz, 5H), 2.09 (t, J=10.1 Hz, 4H), 1.61 (d, J=7.2 Hz, 5H), 1.51 (t, J=9.0 Hz, 5H), 1.46 (dd, J=7.0, 1.5 Hz, 3H), 1.41 (t, J=7.3 Hz, 2H), 1.18-1.11 (m, 7H), 1.05 (s, 9H), 0.99 (s, 3H).

### Example 17: N¹-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N¹³—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)tridecanediamide (56)

Synthesized by using the same procedure used for Example 10 except acid 47 was replaced with acid 48 in 52% yield. ¹H NMR (600 MHz, Chloroform-d) δ 8.67 (s, 1H), 8.32 (d, J=2.0 Hz, 1H), 8.08 (dt, J=9.3, 2.1 Hz, 1H), 7.72 (d, J=8.5 Hz, 2H), 7.41-7.33 (m, 7H), 7.32-7.27 (m, 5H), 7.00 (d, J=7.7 Hz, 3H), 6.67 (d, J=8.5 Hz, 2H), 6.62 (d, J=9.3 Hz, 1H), 6.29 (d, J=8.8 Hz, 1H), 5.07 (t, J=7.2 Hz, 1H), 4.72-4.69 (m, 1H), 4.65 (dd, J=8.9, 1.7 Hz, 1H), 4.51 (d, J=4.0 Hz, 1H), 4.13 (d, J=11.4 Hz, 1H), 3.90 (s, 1H), 3.66 (s, 4H), 3.62-3.58 (m, 1H), 3.27 (s, 4H), 3.18 (s, 2H), 3.10 (dd, J=13.9, 5.1 Hz, 2H), 3.02 (dd, J=13.8, 7.2 Hz, 2H), 2.51 (m, 6H), 2.38 (m, 8H), 2.18 (m, 8H), 2.09-2.02 (m, 3H), 1.68 (s, 2H), 1.60 (d, J=6.2 Hz, 3H), 1.53 (d, J=7.7 Hz, 4H), 1.46 (dd, J=6.9, 1.9 Hz, 3H), 1.23 (d, J=7.2 Hz, 2H), 1.15 (d, J=7.2 Hz, 12H), 1.05 (s, 9H), 1.01-0.96 (m, 3H).

### General Preparation of 59-60:

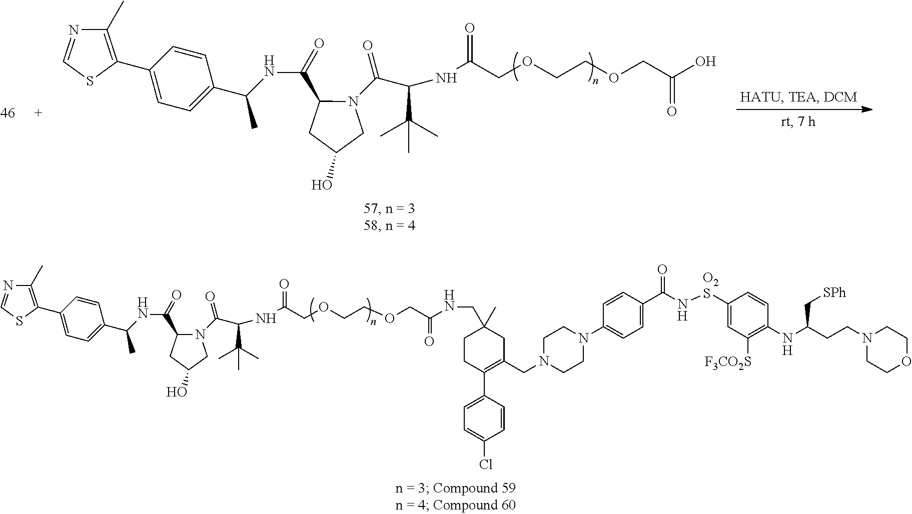

### Example 18: N¹-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N¹⁴—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-3,6,9,12-tetraoxatetradecanediamide (59)

Synthesized by using the same procedure used for Example 10 except acid 47 was replaced with acid 57 in 52% yield. ¹H NMR (600 MHz, Chloroform-d) δ 8.68 (s, 1H), 8.34 (d, J=2.3 Hz, 1H), 8.11 (dd, J=9.4, 2.3 Hz, 1H), 7.68-7.66 (m, 2H), 7.52 (d, J=7.5 Hz, 1H), 7.41-7.35 (m, 7H), 7.32-7.27 (m, 5H), 7.05 (d, J=8.6 Hz, 1H), 7.00 (d, J=7.7 Hz, 3H), 6.76 (d, J=8.6 Hz, 2H), 6.62 (d, J=9.4 Hz, 1H), 5.10 (dd, J=14.2, 7.0 Hz, 2H), 4.74 (t, J=8.0 Hz, 1H), 4.63 (d, J=8.1 Hz, 1H), 4.52 (s, 1H), 4.14 (d, J=11.4 Hz, 1H), 4.04 (s, 3H), 3.93 (d, J=6.4 Hz, 3H), 3.70-3.60 (m, 16H), 3.25 (m, 5H), 3.10 (q, J=7.2 Hz, 4H), 3.02 (dd, J=13.8, 7.2 Hz, 2H), 2.49 (d, J=1.1 Hz, 3H), 2.35 (m, 4H), 2.11 (m, 4H), 2.01 (d, J=17.0 Hz, 3H), 1.71-1.66 (m, 3H), 1.59-1.54 (m, 3H), 1.47 (s, 2H), 1.39 (d, J=7.4 Hz, 4H), 1.06 (s, 9H), 0.99 (d, J=2.5 Hz, 3H).

### Example 19: N¹-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N¹⁷—((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-3,6,9,12,15-pentaoxaheptadecanediamide (60)

Synthesized by using the same procedure used for Example 10 except acid 47 was replaced with acid 58 in 49% yield. ¹H NMR (600 MHz, Chloroform-d) δ 8.70 (s, 1H), 8.35 (s, 1H), 8.14 (dd, J=9.2, 2.3 Hz, 2H), 7.70 (dd, J=9.0, 3.9 Hz, 2H), 7.43-7.37 (m, 7H), 7.33 (d, J=7.7 Hz, 3H), 7.31-7.29 (m, 2H), 7.08 (d, J=8.6 Hz, 2H), 7.02 (d, J=8.1 Hz, 2H), 6.79 (d, J=8.7 Hz, 2H), 6.64 (d, J=9.3 Hz, 2H), 5.13 (d, J=17.0 Hz, 2H), 4.77 (s, 2H), 4.65 (dd, J=8.6, 2.9 Hz, 1H), 4.54 (s, 1H), 4.16 (d, J=11.4 Hz, 1H), 4.06 (s, 2H), 4.00 (d, J=6.6 Hz, 2H), 3.95 (dd, J=15.5, 8.4 Hz, 3H), 3.73-3.59 (m, 20H), 3.37 (s, 2H), 3.26 (s, 4H), 3.04 (dd, J=13.9, 7.2 Hz, 3H), 2.82 (s, 3H), 2.54-2.51 (m, 3H), 2.44 (s, 3H), 2.37-2.32 (m, 4H), 2.12 (m, 4H), 2.04 (t, J=13.6 Hz, 3H), 1.50 (d, J=6.9 Hz, 4H), 1.09 (s, 9H), 1.02 (s, 3H).

### Example 20: 4-(4-((4′-chloro-4-((6-((2-(2,6-dioxopiperidin-3-yl)-1,3-dioxoisoindolin-4-yl)amino)hexanamido)methyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (62)

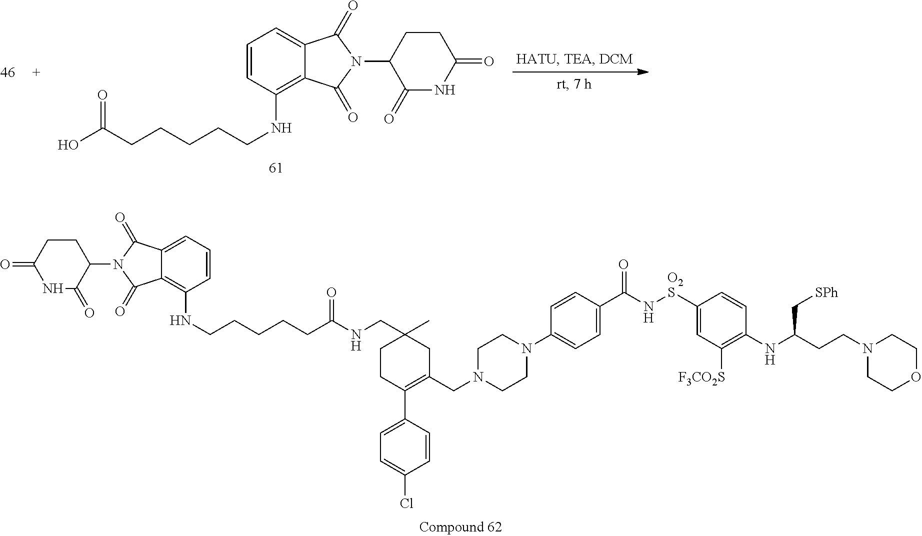

Synthesized by using the same procedure used for Example 10 except acid 47 was replaced with acid 61 in 47% yield. ¹H NMR (600 MHz, Chloroform-d) δ 8.88 (s, 1H), 8.36 (d, J=2.3 Hz, 1H), 8.06-8.02 (m, 1H), 7.63 (d, J=8.5 Hz, 2H), 7.40-7.34 (m, 5H), 7.31 (ddd, J=7.6, 6.5, 1.2 Hz, 2H), 7.25 (s, 1H), 7.02 (dt, J=9.1, 1.9 Hz, 2H), 6.97-6.89 (m, 2H), 6.71 (s, 1H), 6.66 (d, J=9.2 Hz, 1H), 6.41 (s, 1H), 5.96 (s, 1H), 4.93-4.81 (m, 1H), 3.92 (s, 1H), 3.66 (s, 5H), 3.54 (s, 2H), 3.21-3.08 (m, 4H), 3.02 (dd, J=13.8, 7.2 Hz, 2H), 2.94 (s, 3H), 2.85 (dd, J=12.7, 2.8 Hz, 2H), 2.73 (d, J=11.1 Hz, 3H), 2.49-2.28 (m, 11H), 2.22 (t, J=7.4 Hz, 3H), 2.10 (d, J=11.6 Hz, 3H), 1.71-1.58 (m, 5H), 1.54 (d, J=6.6 Hz, 3H), 1.34-1.15 (m, 3H), 1.03-0.99 (m, 3H).

### Example 21: Cell Viability Assay

Cancer cells from different tissue origins including acute lymphoblastic leukemia (MOLT4 and RS4; 11), small cell lung cancer (NCI-H146 or simply H146), and multiple myeloma (EJM and H929) were incubated with increasing concentrations of Examples 1-21 or ABT-263 for 72 h. Cell viability was measured by tetrazolium-based MTS assay. 5×10⁴ to 1×10⁵ suspension cells or 3×10³ to 5×10³ adherent cells were seeded and treated in 96-well plates for 72 h. The EC₅₀ values of individual agents were calculated with GraphPad Prism.

### Example 22: Protein Degradation Assays in MOLT4 Cells and Human Platelets

MOLT4 cells and human platelets were incubated with increasing concentrations of test compounds for 16 h. The cells were harvested and lysed in RIPA lysis buffer supplemented with protease and phosphatase inhibitor cocktails. An equal amount of protein (20 μg/lane) was resolved on a pre-cast 4-20% SDS-PAGE gel. Proteins were subsequently transferred to NOVEX PVDF membranes by electrophoresis. The membranes were blocked in blocking buffer (5% non-fat dry milk in TBS-T), and incubated with primary antibodies (at optimized concentrations) overnight at 4° C. After three washings in TBS-T, the membranes were incubated with an appropriate HRP-conjugated secondary antibody for 1 h at room temperature. After extensive washing for three times, the proteins of interest were detected with ECL western blotting detection reagents and recorded with autoradiography (Pierce Biotech, Rockford, Ill., USA). The primary antibodies for Bcl-xL (Cat #2762), Bcl-2 (Cat #2872), Mcl-1 (Cat #5453) and β-actin (Cat #4970) were purchased from Cell Signaling technology. The relative band intensity was measured using ImageJ software and normalized to b-actin. The DC₅₀ (concentration with 50% degradation) was calculated using GraphPad Prism.

### Example 23: Ternary Complex Assay

To detect ternary complex formation induced by the compounds, AlphaLISA assay was used to measure luminescence signals arisen from proximity of BCL-X_{L} bounded acceptor beads and VHL- or CRBN-bounded donor beads. Briefly, to a 96-well PCR plate, 10 μL of 20 nM 6-His tagged BCL-X_{L} protein was mixed with 10 μL of 20 nM GST-tagged VHL complex protein and 10 μL of serially diluted testing compounds. After incubating at room temperature for 30 min, 5 μL of 160 μg/mL Glutathione donor beads (PerkinElmer) was added and the mixture was incubated at dark for 15 min. 5 μL of 160 μg/mL of anti-His acceptor beads were added lastly and the mixture was incubated for an additional 45 min before transferred to two adjacent wells (17 μL each) of 384-well white OptiPlate (PerkinElmer). The luminescence signals were detected on a Biotek's Synergy Neo2 multi-mode plate reader installed with an AphaScreen filter cube. All reagents were diluted in an assay buffer of 25 mM FIEPES, pH 7.5, 100 mM NaCL, 0.1% BSA, and 0.005%5 tween 20 prior incubation.

### Results

### Compounds of the Invention Induce Degradation of BCL-X_{L} in MOLT-4 and RS4 Cells

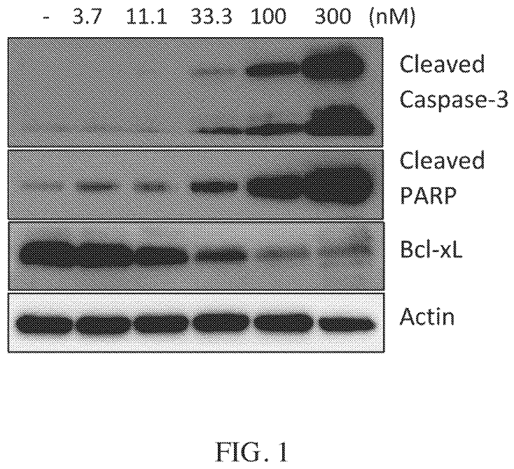

TABLE 1
demonstrates that various compounds of Formula (I) degradation of BCL-X_{L} in MOLT-4 and RS4 cells, and is further illustrated in Figure 1.

| | | MOLT-4 | RS4 |
|---|---|---|---|
| Cmpd | | IC₅₀ | IC₅₀ |
| No. | Compound Structure | (nM) | (nM) |
| 62 | 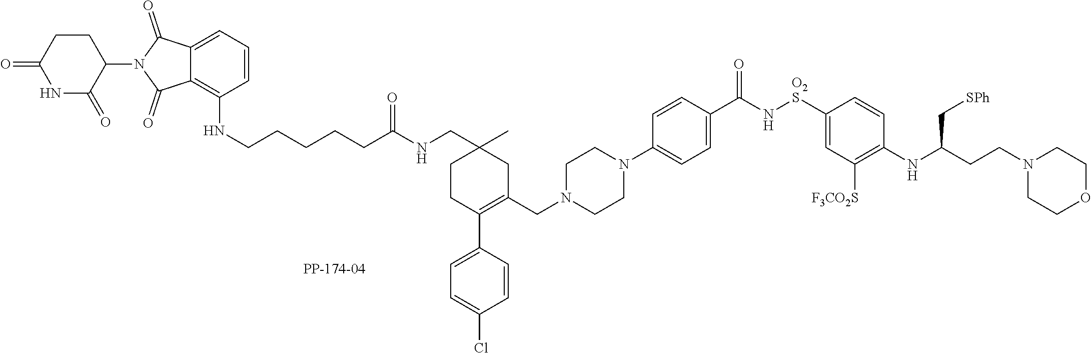 | 36 | 33 |
| 50 | 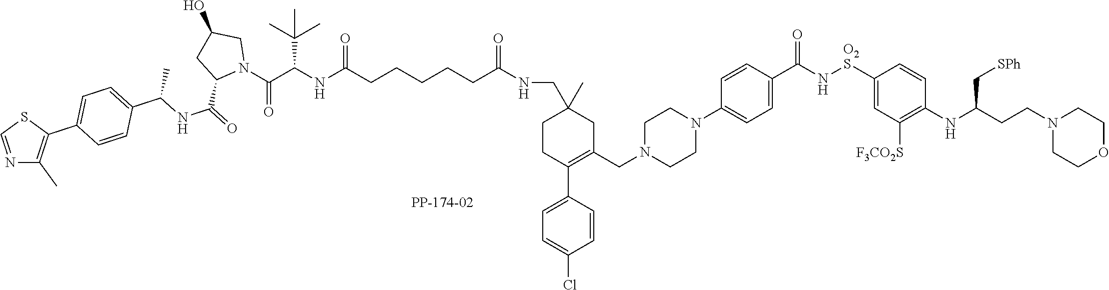 | 187 | 99 |
| 51 | 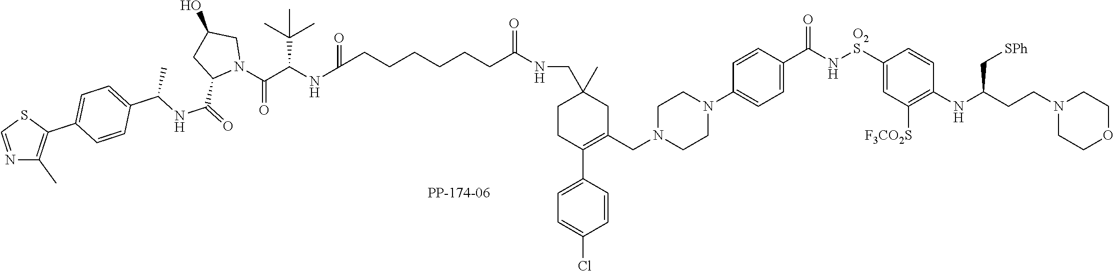 | 142 | 47 |
| 49 | 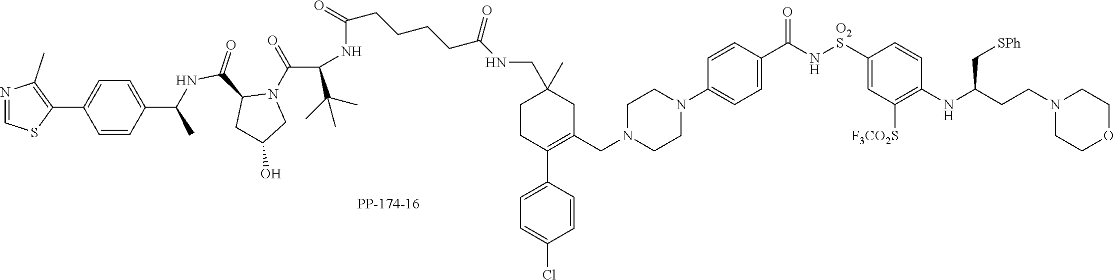 | 340 | 303 |
| 52 | 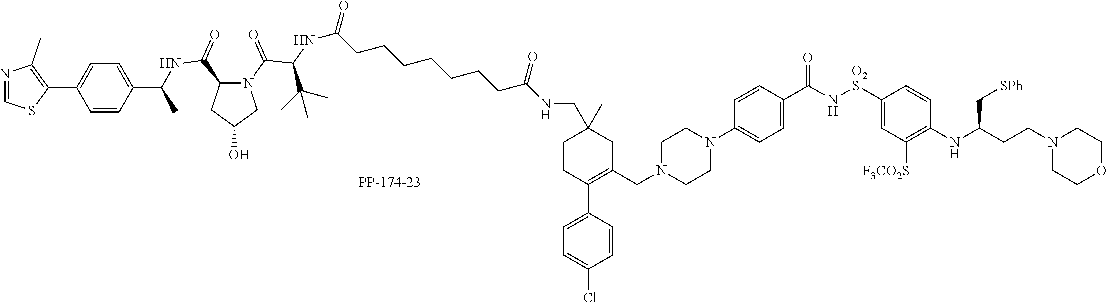 | 124 | 43 |
| 53 | 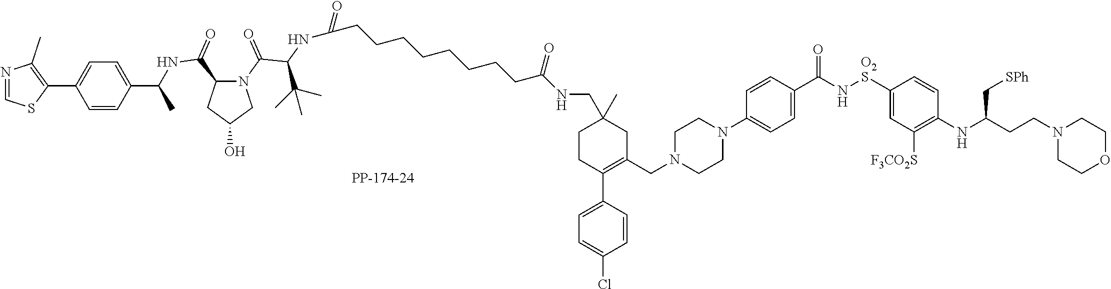 | 36 | 17.4 |
| 54 | 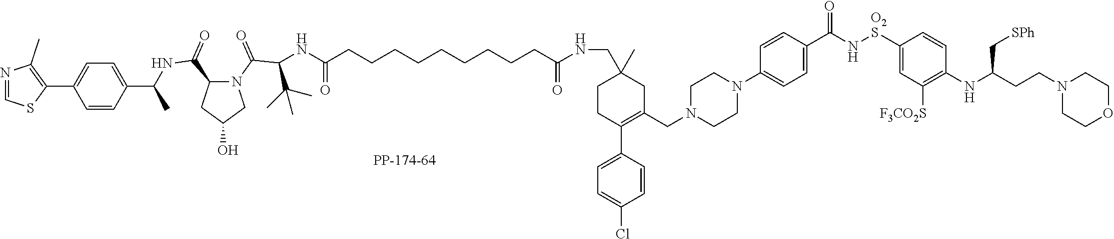 | 74 | 19.4 |
| 55 | 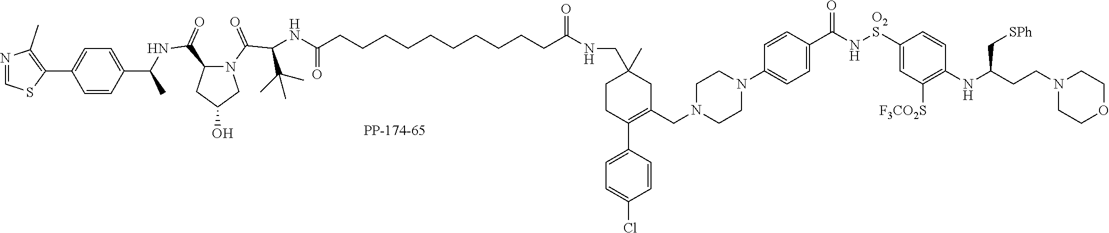 | 111 | 96 |
| 56 | 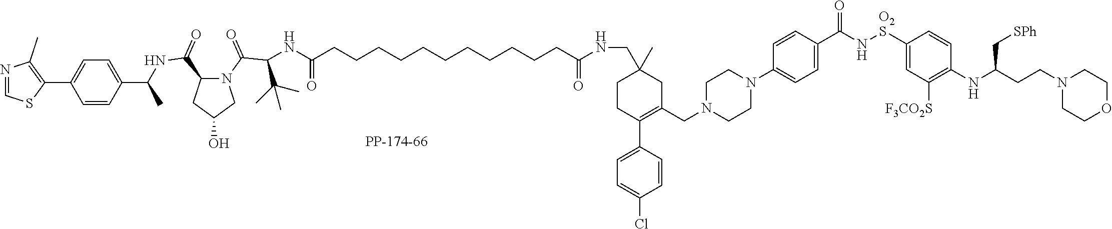 | 333 | 410 |
| 59 | 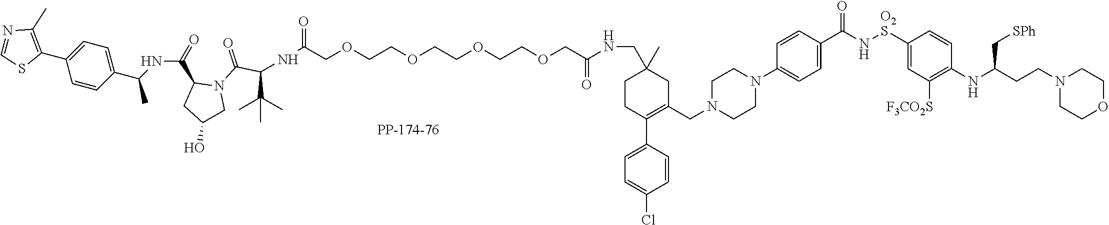 | 973 | 404 |
| 60 | 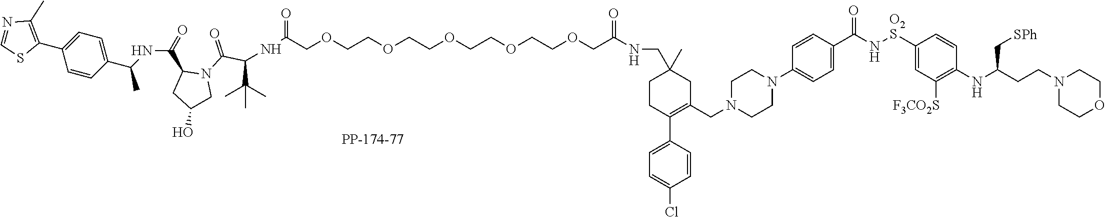 | 1411 | 758 |
| 22 | 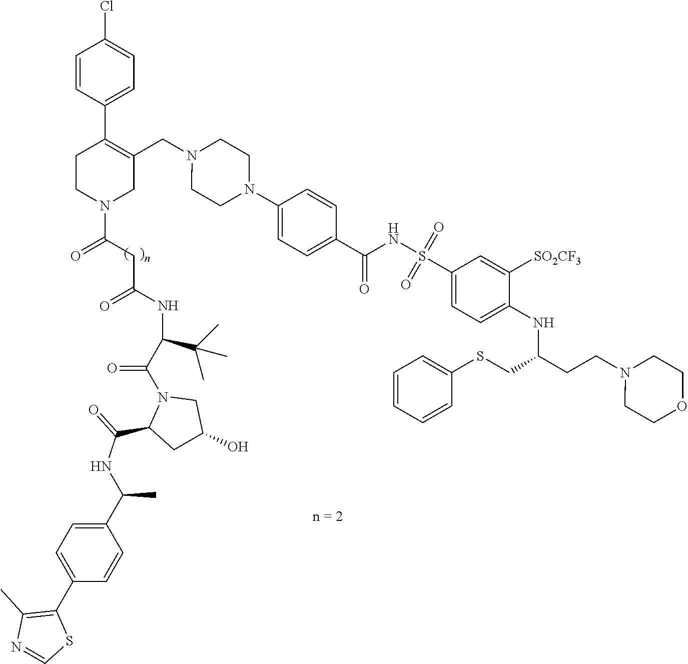 | >2000 | >2000 |
| 23 | 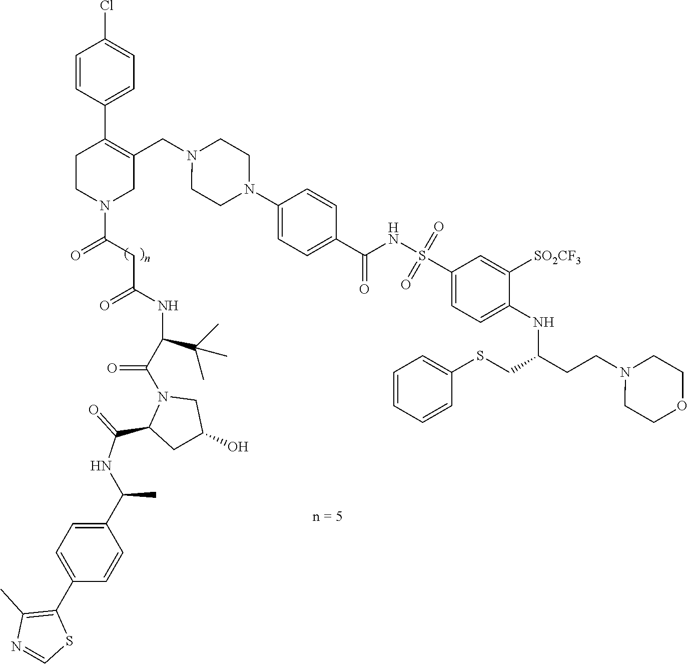 | >2000 | 810 |
| 24 | 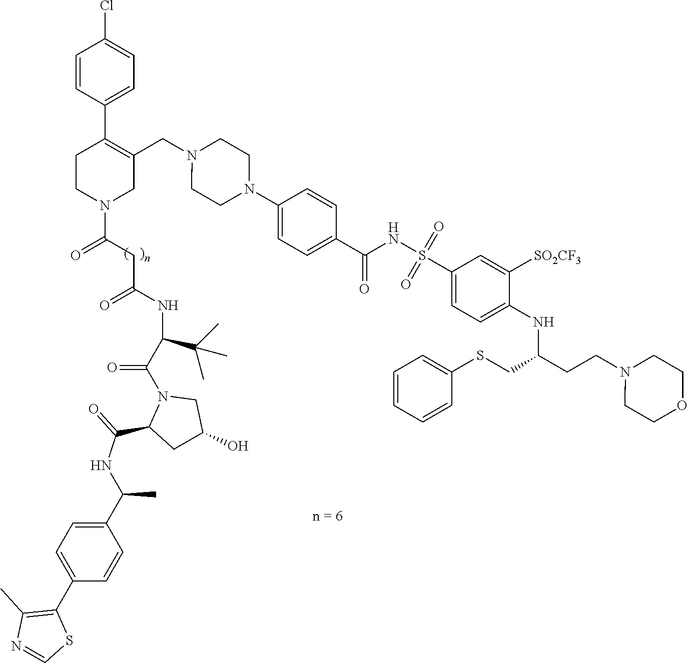 | 1043 | 611 |
| 25 | 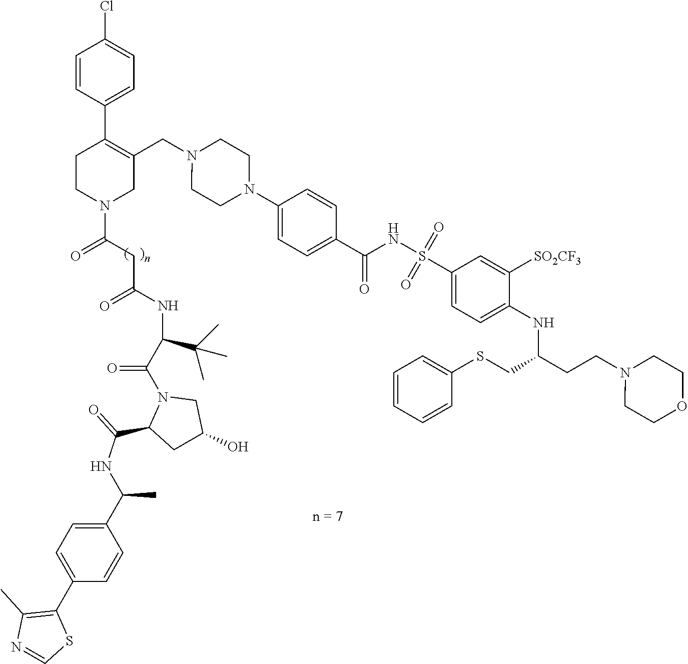 | 539 | 1195 |
| 26 | 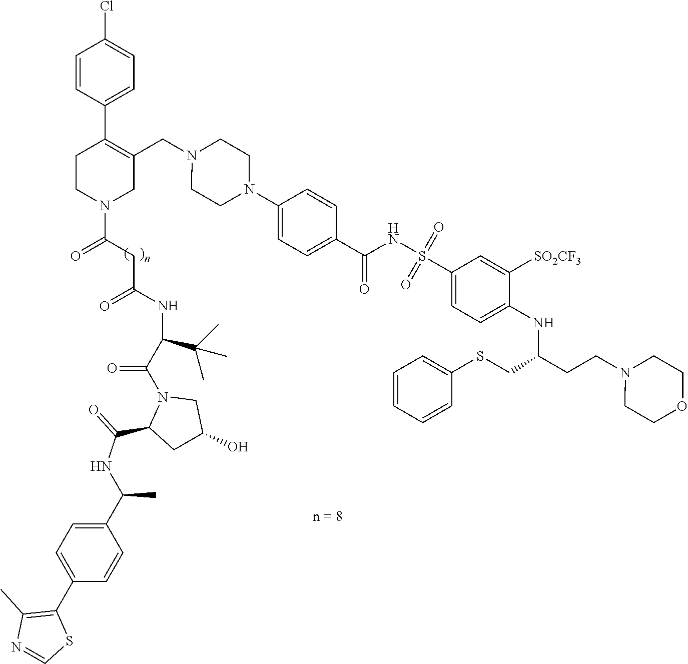 | 70.4 | 226 |
| 27 | 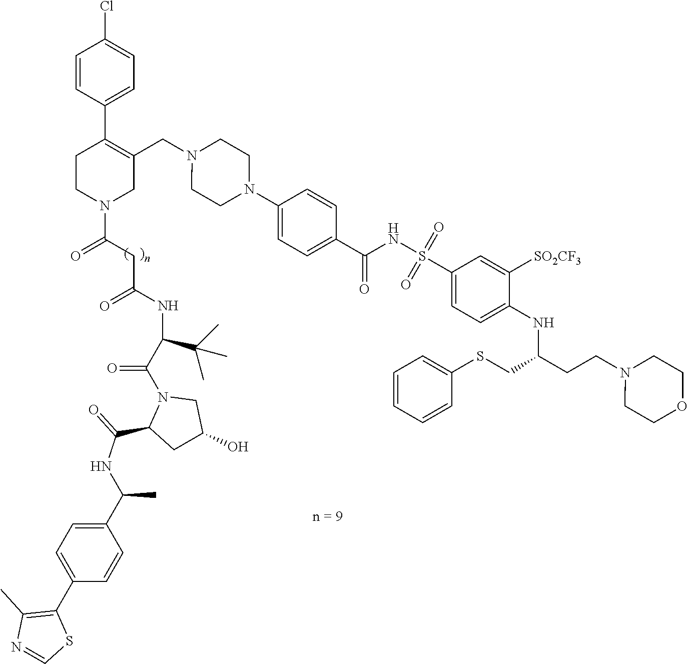 | 202 | 583 |
| 28 | 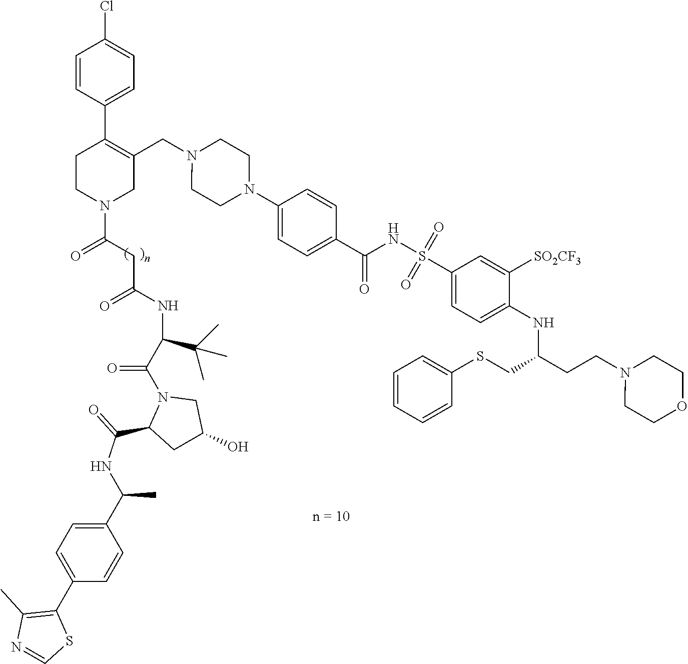 | 130 | 400 |
| 35 | 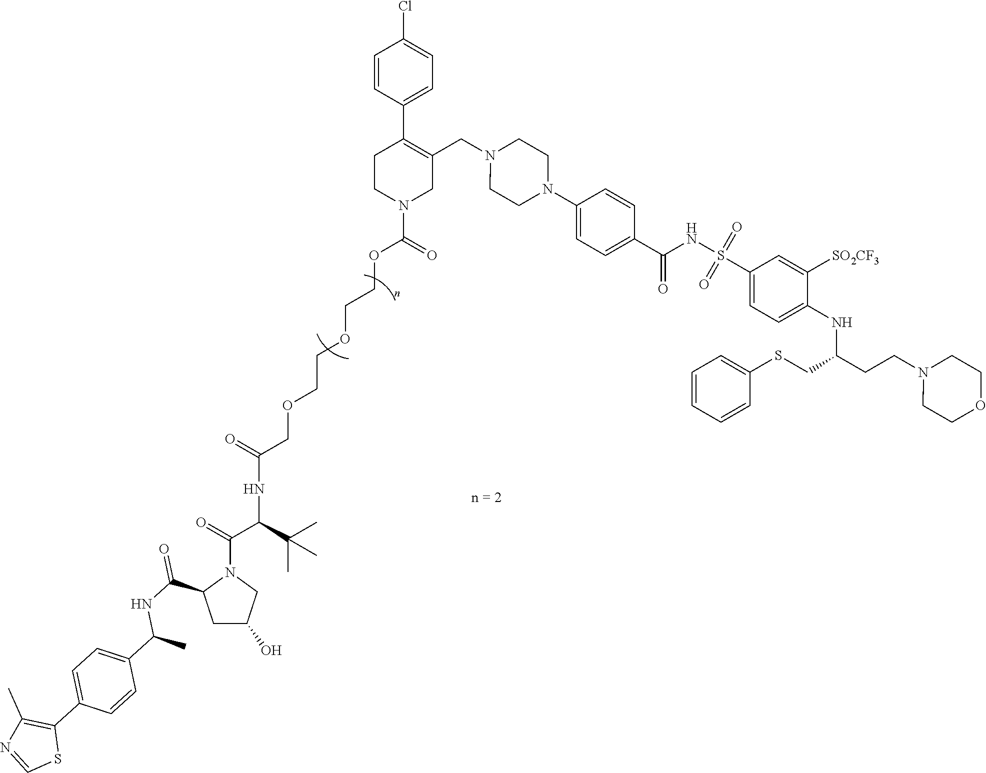 | 566 | 2059 |
| 36 | 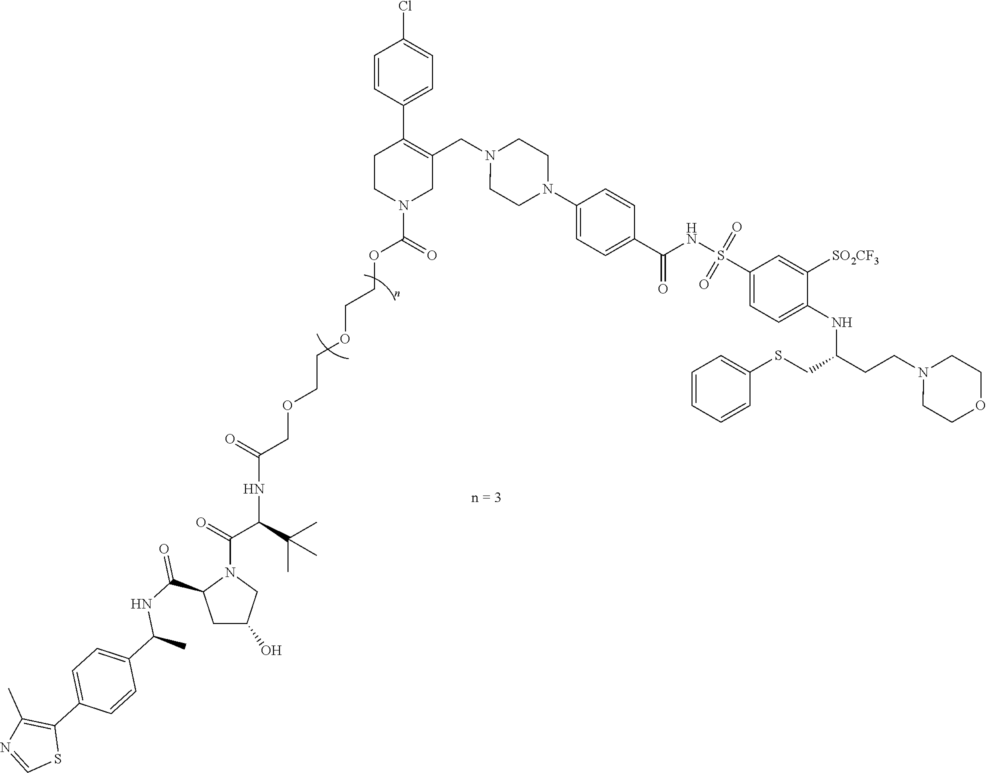 | 342 | 1748 |

### Compounds of the Invention Reduce the On-Target Toxicity (Thrombocytopenia) Relative to ABT-263

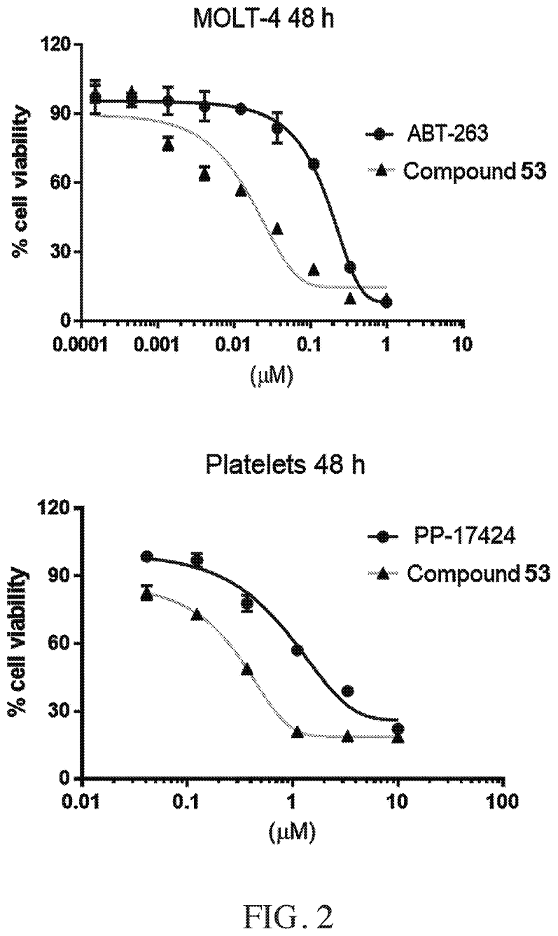

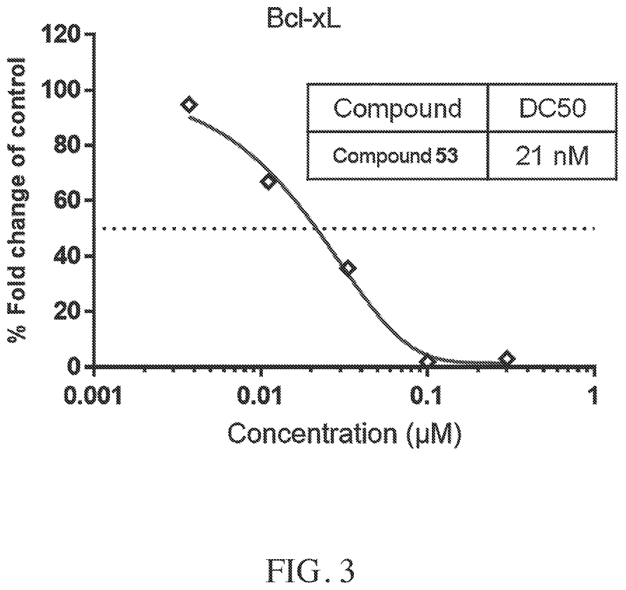

FIG. 2 depicts the inhibitory effects of both Compound 53 and ABT-263 on MOLT-4 cells and human platelets. Compound 53 is both more potent than ABT-263 in MOLT-4 cells (anti-cancer effect), but also demonstrates substantially less effects on human platelets than ABT-263, as summarized below in Table 2. Thus, the compounds of the present invention (e.g., Formula (I)) possess a far superior therapeutic index (e.g., Platelet/MOLT-4 ratio) than representative Bcl-2 inhibitors in the art (e.g., ABT-263).

TABLE 2

| | MOLT-4 | Platelets | Ratio Platelate/ |
|---|---|---|---|
| Compound | IC₅₀ nM | IC₅₀ nM | MOLT-4 |
| ABT-263 | 202 | 190 | 0.9 |
| Compound 53 | 36 | 1553 | 43 |

### Compounds of the Invention Form Ternary Complexes with VHL Complex and BCL-X_{L} and Induce BCL-X_{L} Degradation

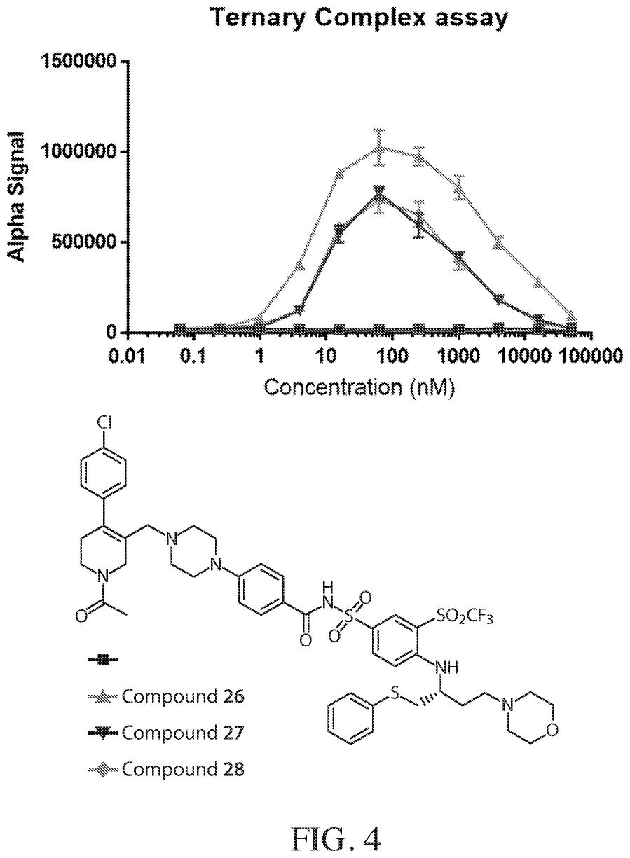

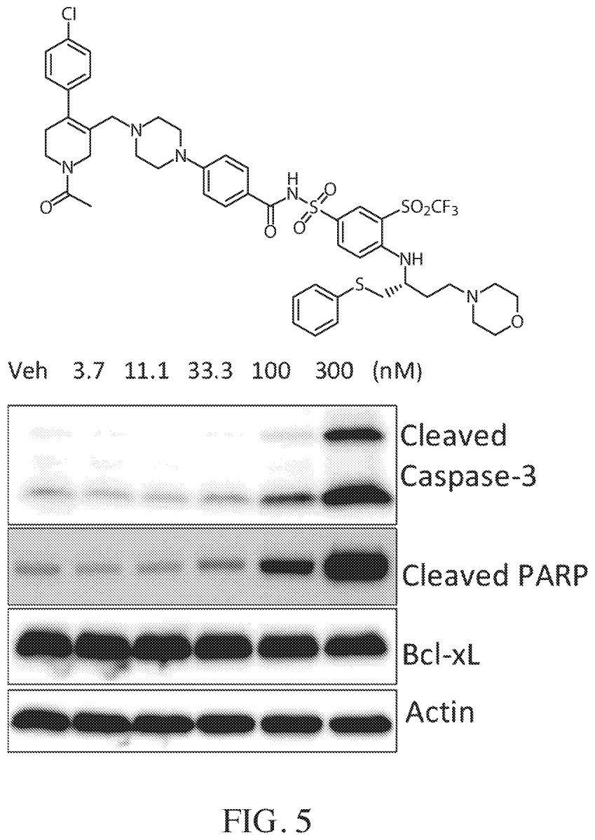

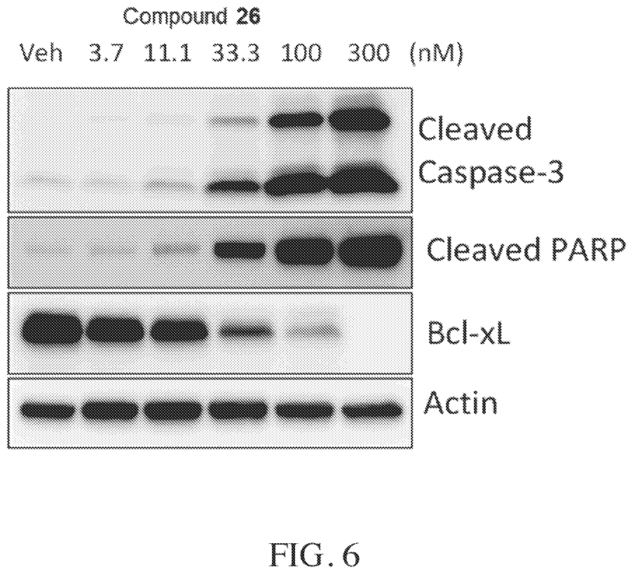

Compounds of the invention form ternary complexes with the VHL-complex and BCL-XL while non-PROTAC compounds do not (FIG. 4). Similarly, compounds of the invention induce BCL-XL degradation (FIG. 6), while the non-PROTAC compounds do not (FIG. 5).

TABLE 3
Exemplary degraders of the present disclosure and their IC₅₀ against MOLT-4 and RS4;11 cells.

| | | IC₅₀ (48 | |
|---|---|---|---|
| De- | | hours)^{a} | |
| grader | | MOL | RS4; |
| # | Structure | T-4 | 11 |
| 1 | 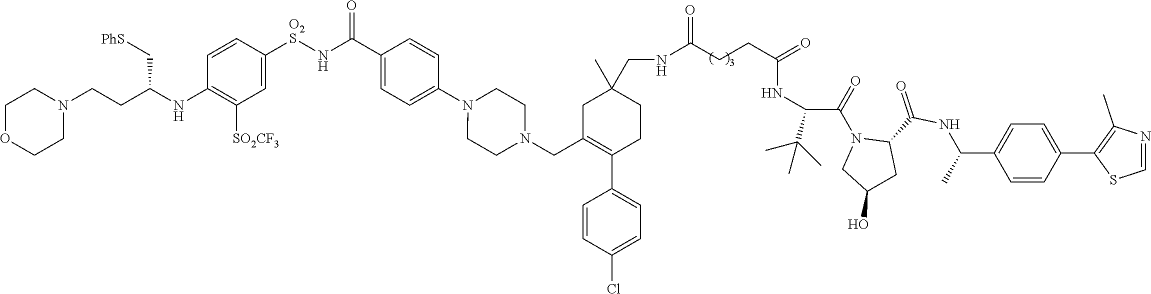 | ++ | ++ |
| 2 | 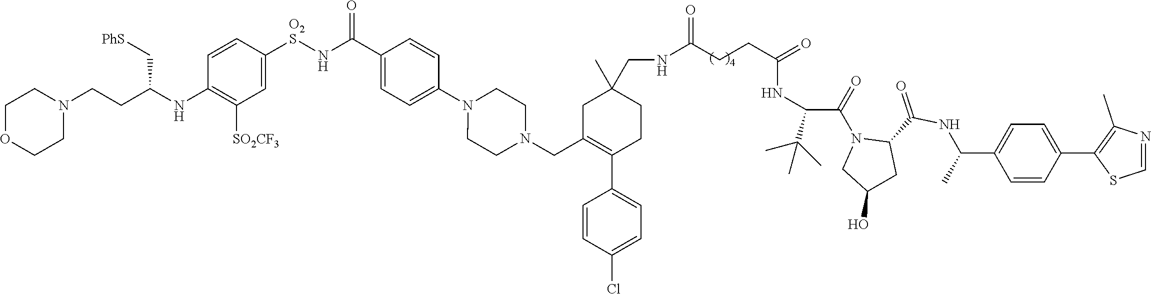 | ++ | ++ |
| 3 | 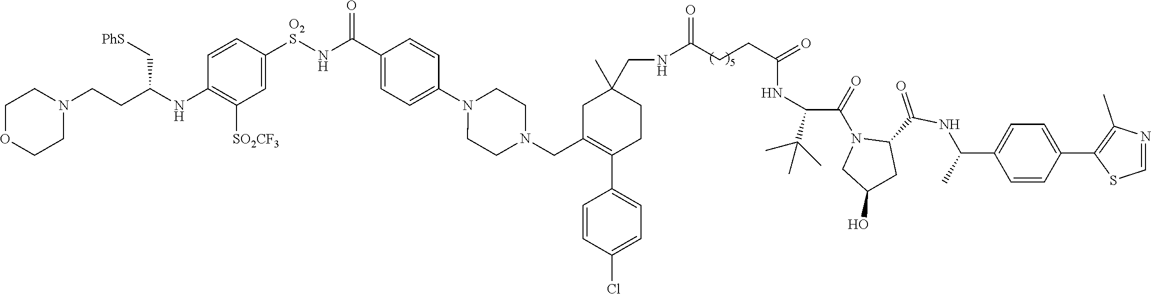 | ++ | +++ |
| 4 | 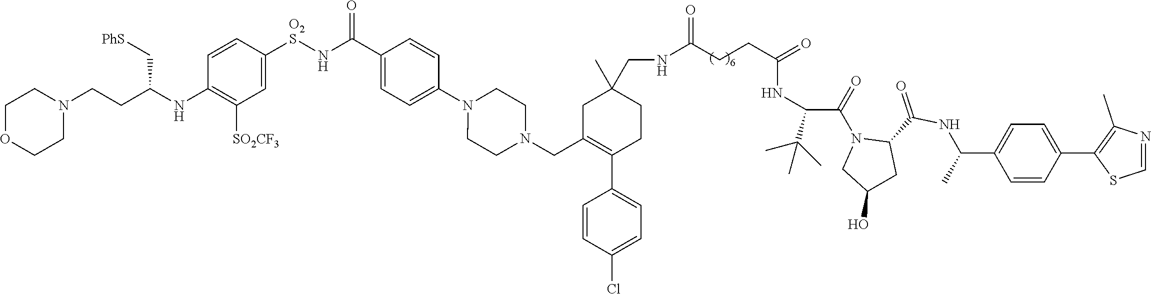 | +++ | ++++ |
| 5 | 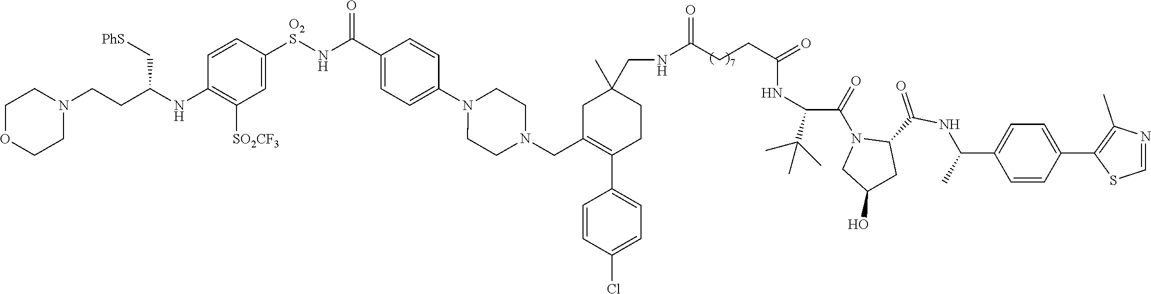 | ++++ | ++++ |
| 6 | 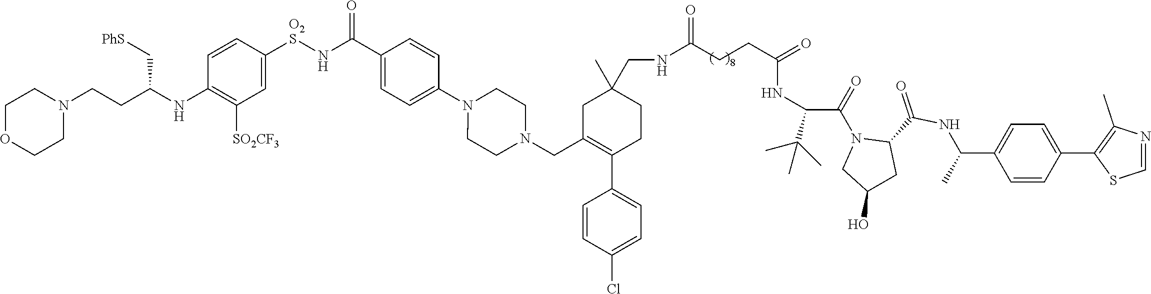 | +++ | ++++ |
| 7 | 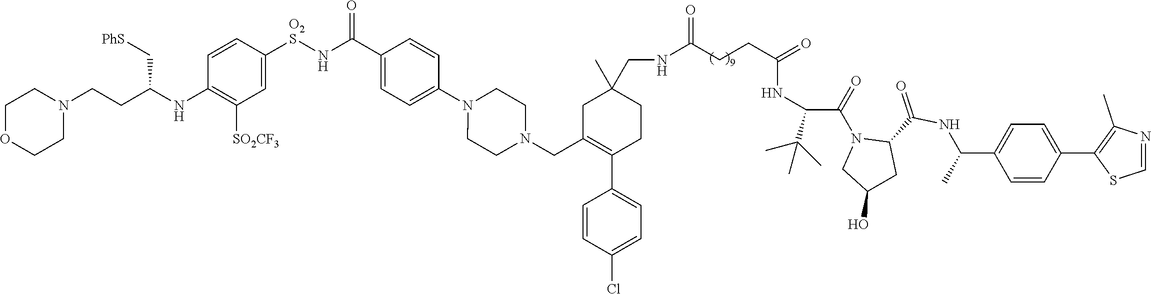 | +++ | +++ |
| 8 | 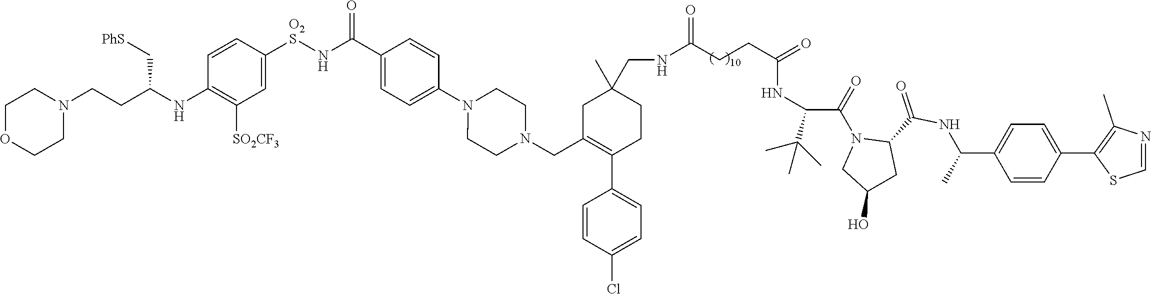 | ++ | ++ |
| 9 | 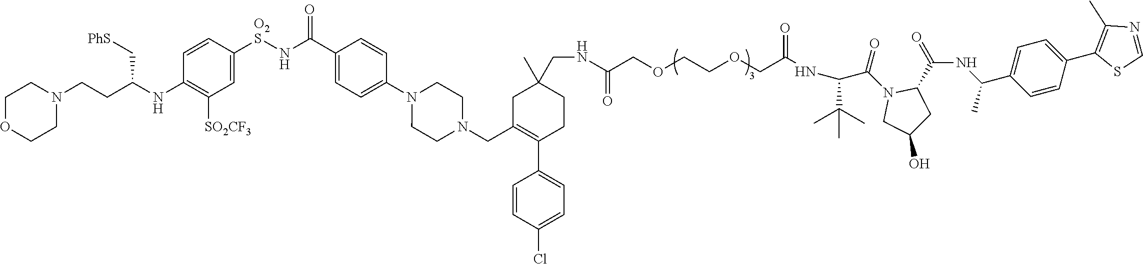 | + | ++ |
| 10 | 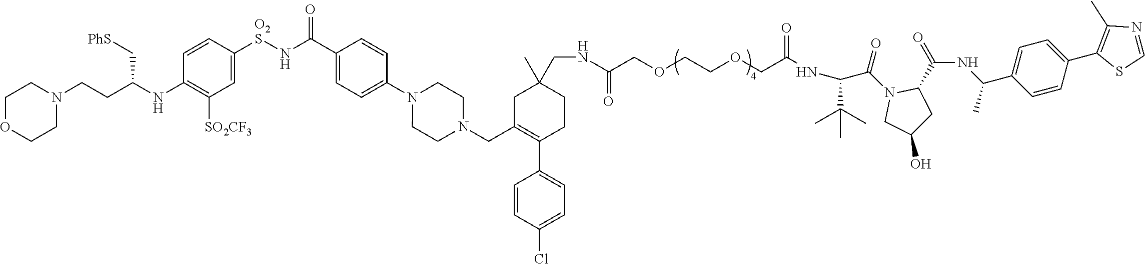 | + | + |
| 11 | 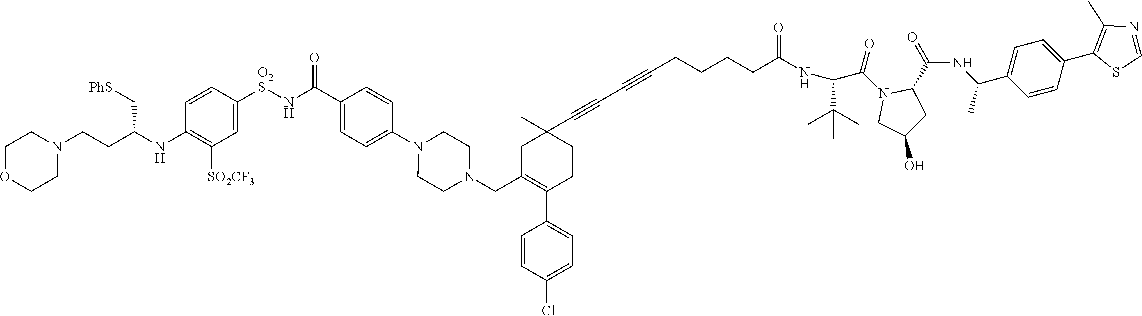 | + | + |
| 12 | 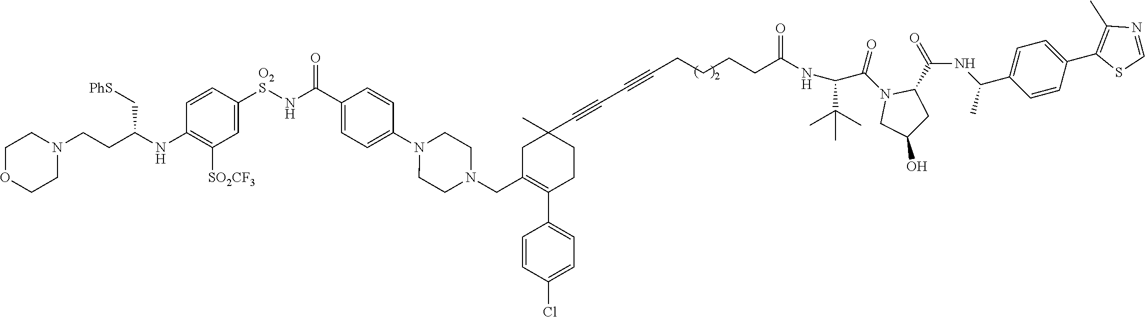 | + | + |
| 13 | 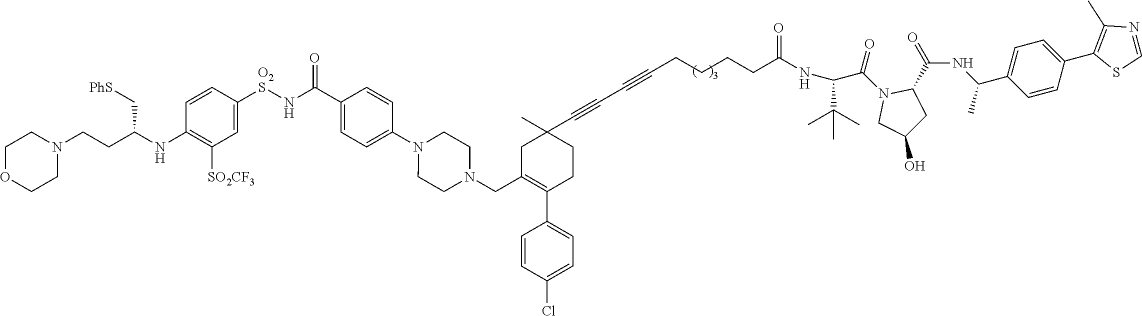 | + | + |
| 14 | 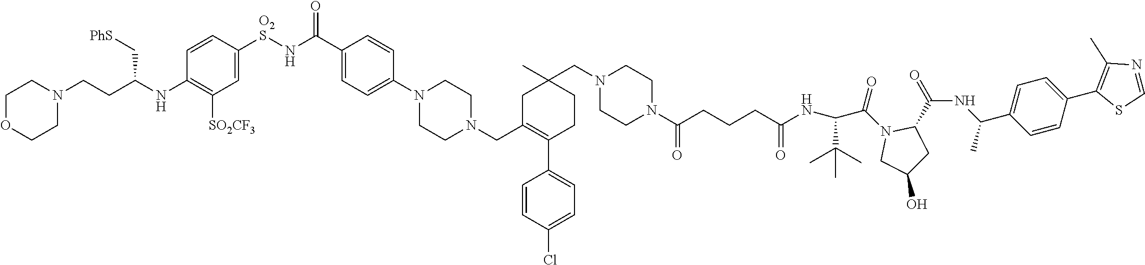 | ++ | +++ |
| 15 | 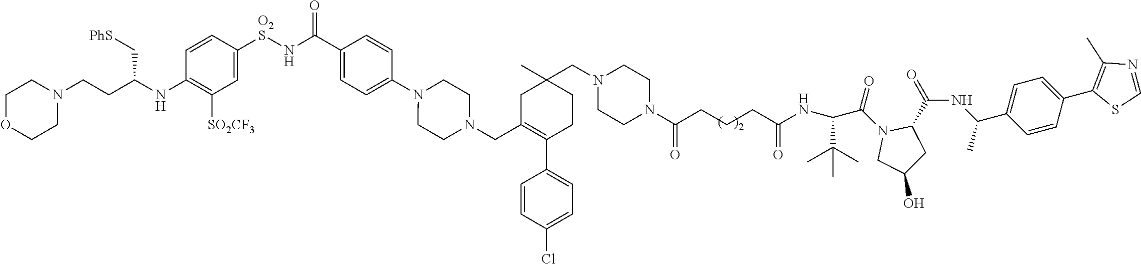 | +++ | +++ |
| 16 | 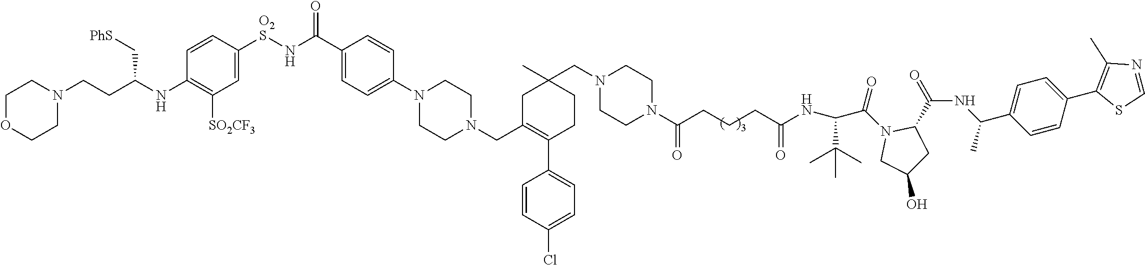 | ++++ | ++++ |
| 17 | 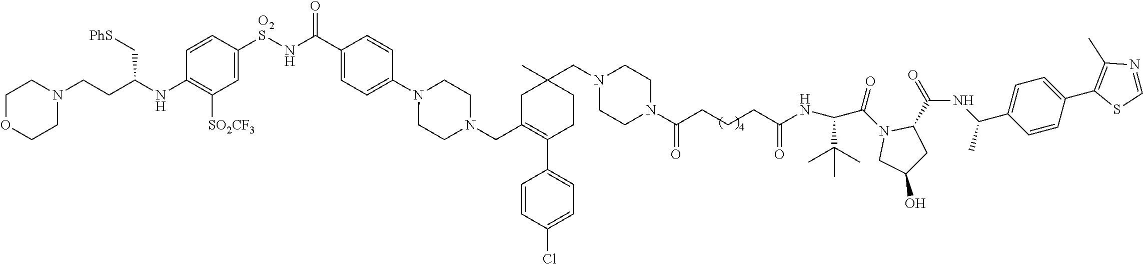 | ++++ | ++++ |
| 18 | 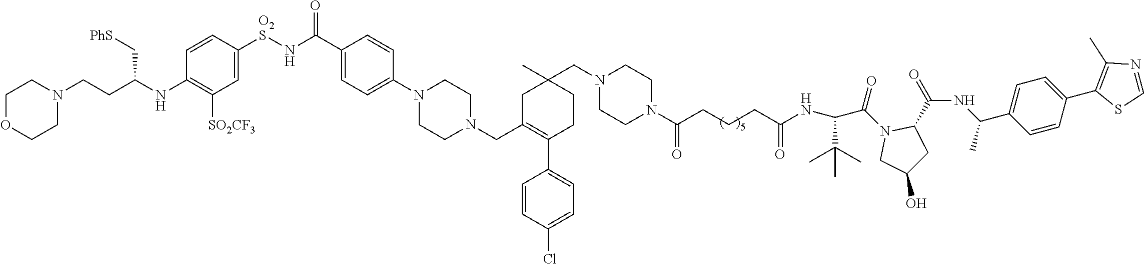 | ++++ | ++++ |
| 19 | 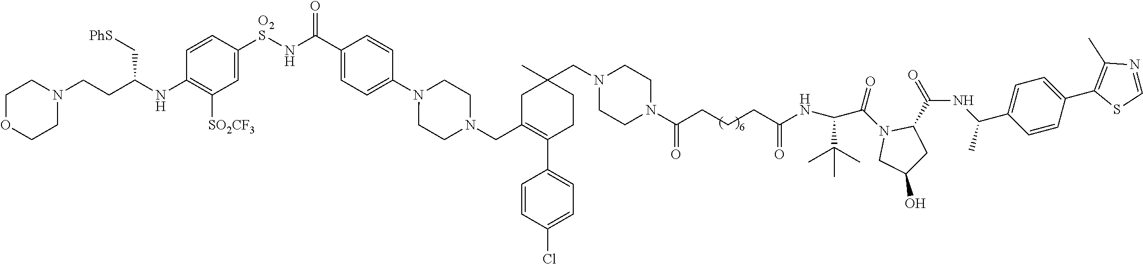 | +++ | +++ |
| 20 | 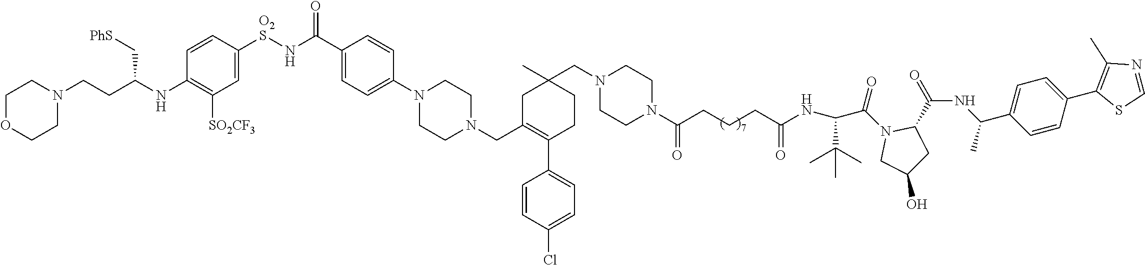 | ++ | ++ |
| 21 | 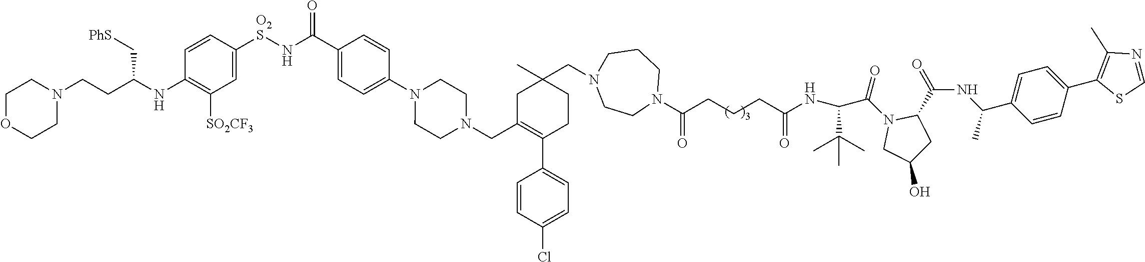 | +++ | ++++ |
| 22 | 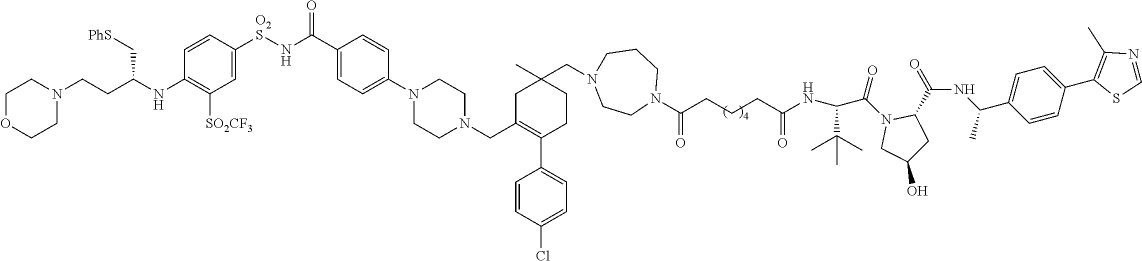 | ++++ | ++++ |
| 23 | 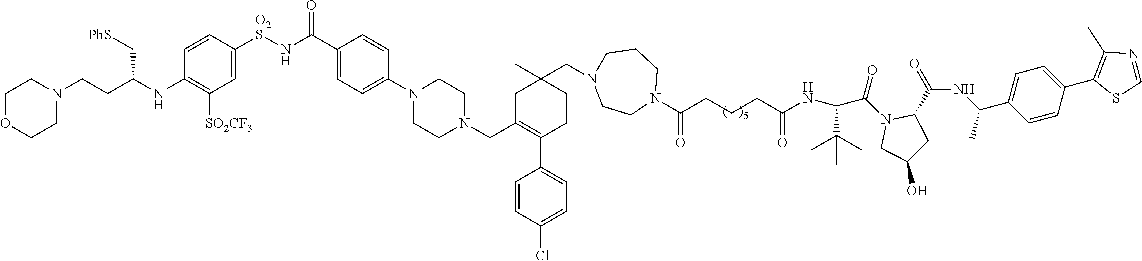 | +++ | ++++ |
| 24 | 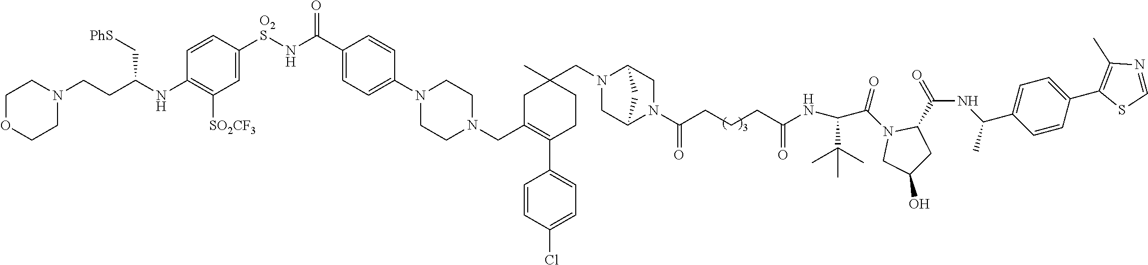 | +++ | ++++ |
| 25 | 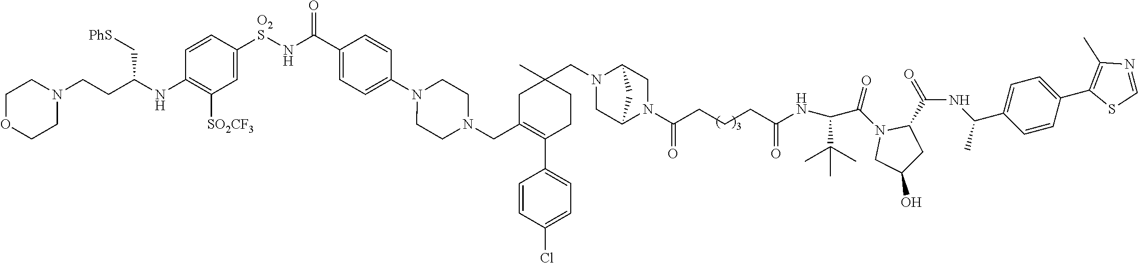 | ++++ | ++++ |
| 26 | 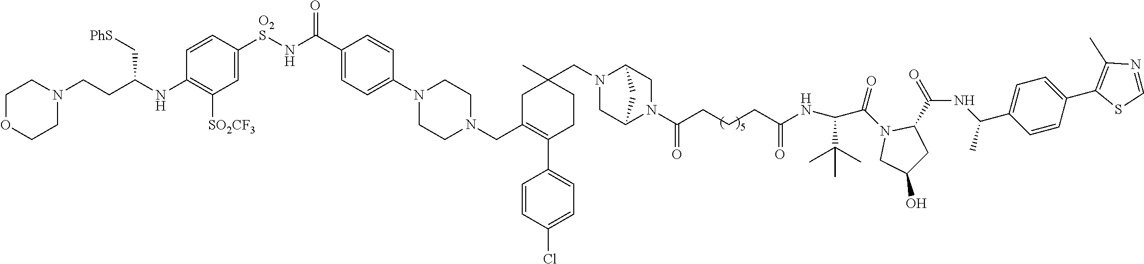 | ++++ | ++++ |
| 27 | 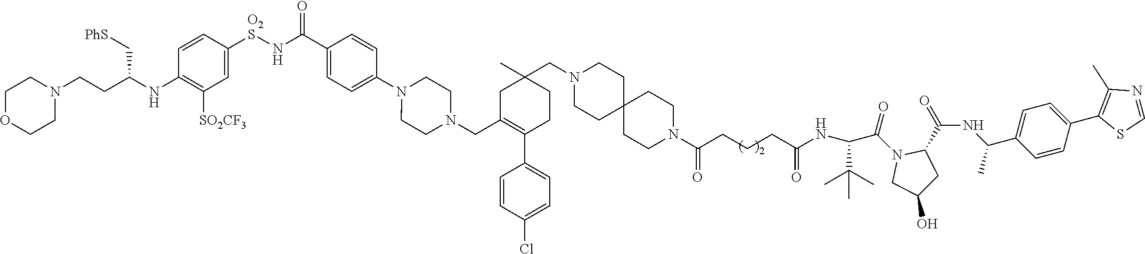 | ++++ | ++++ |
| 28 | 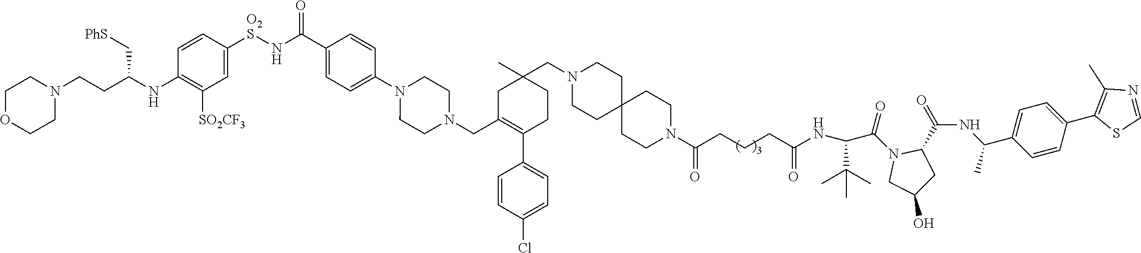 | ++++ | ++++ |
| 29 | 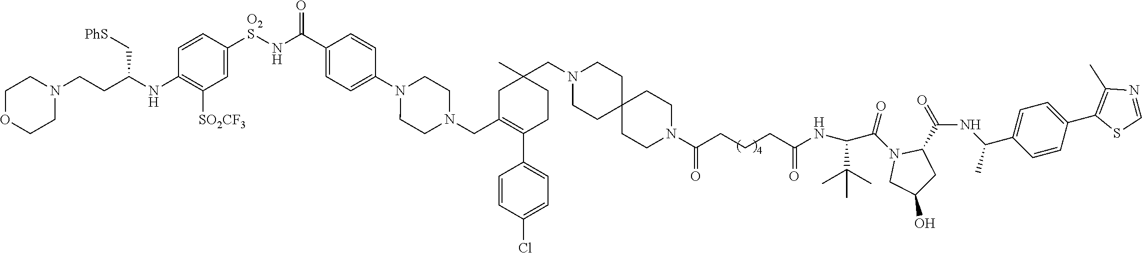 | ++++ | ++++ |
| 30 | 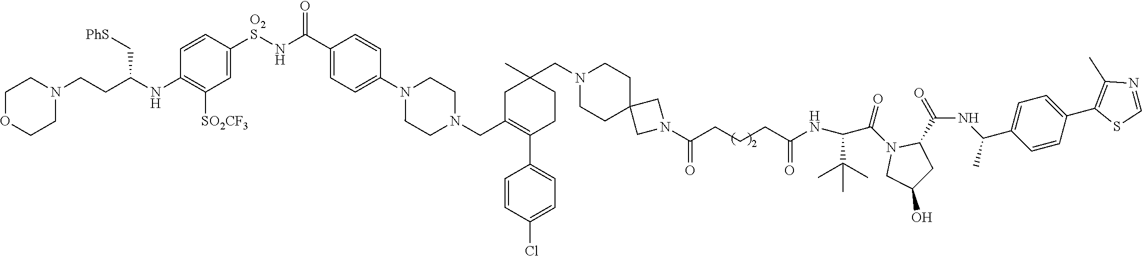 | +++ | ++++ |
| 31 | 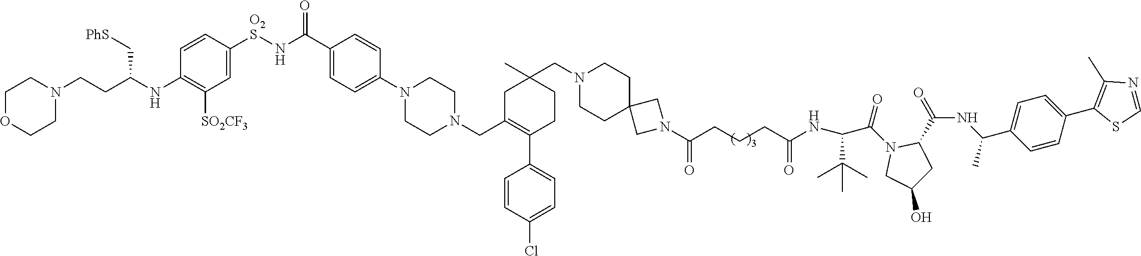 | +++ | ++++ |
| 32 | 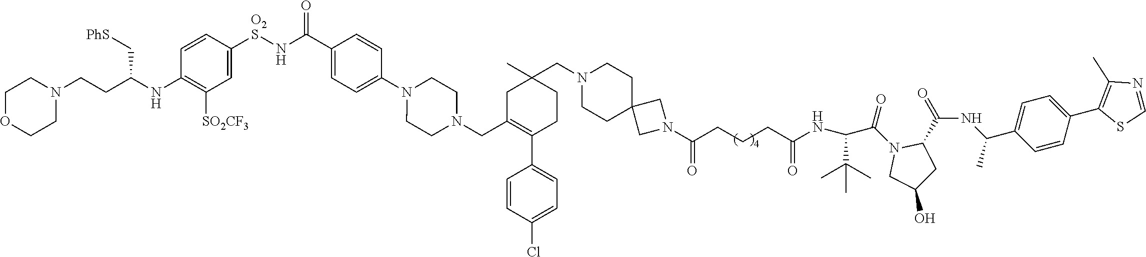 | +++ | ++++ |
| 33 | 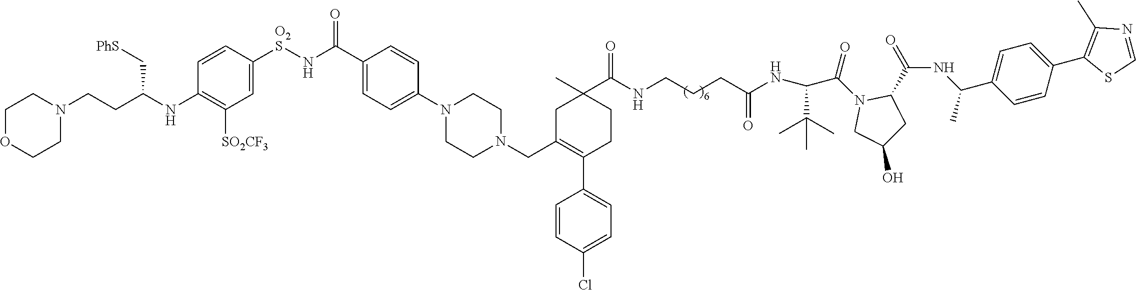 | ++ | +++ |
| 34 | 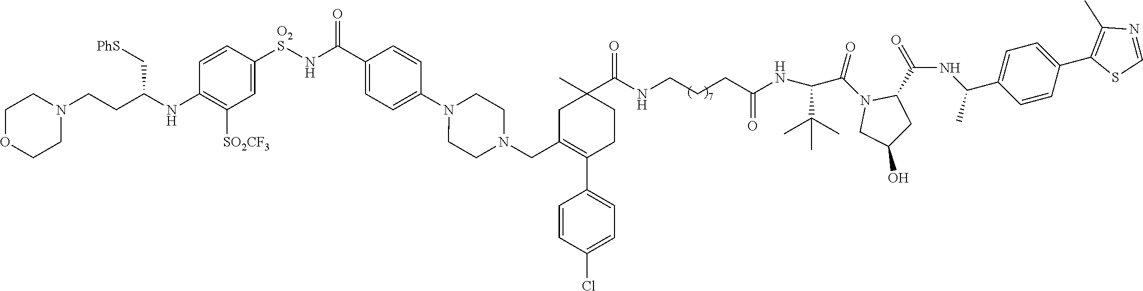 | +++ | +++ |
| 35 | 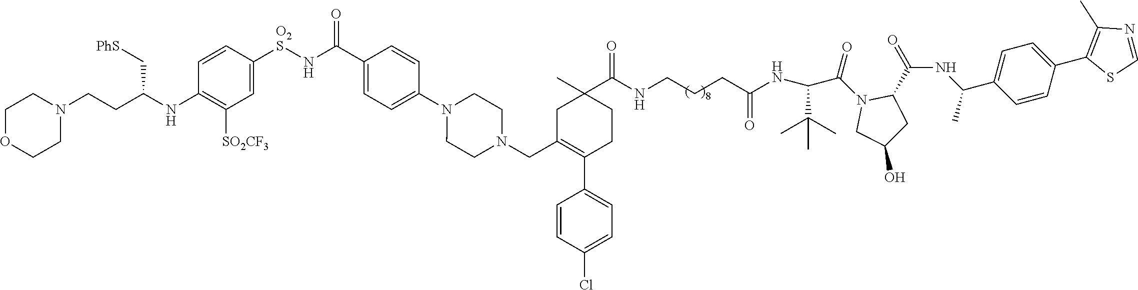 | ++ | ++ |
| 36 | 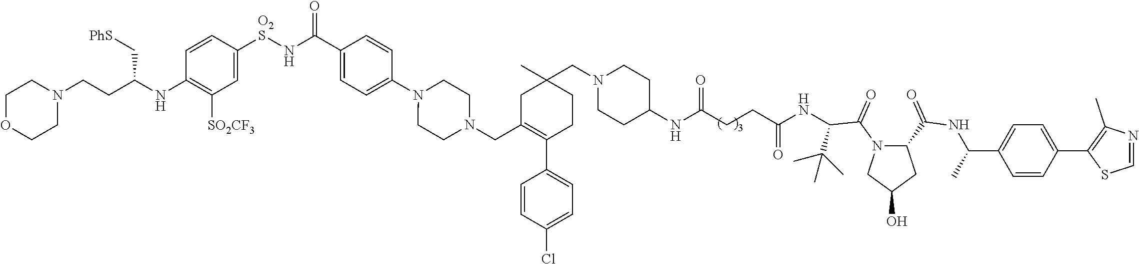 | ++ | +++ |
| 37 | 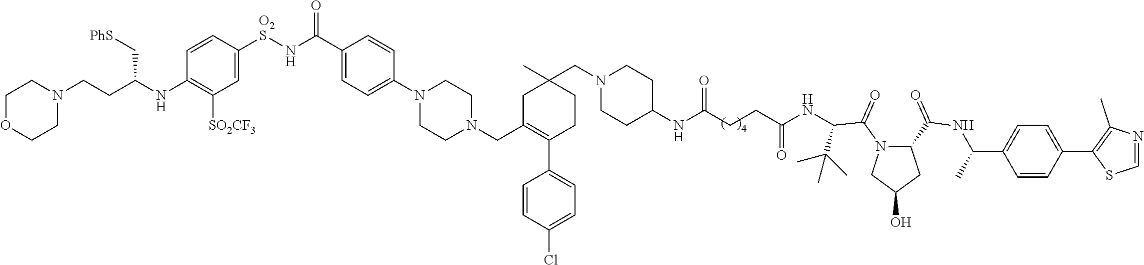 | ++ | ++++ |
| 38 | 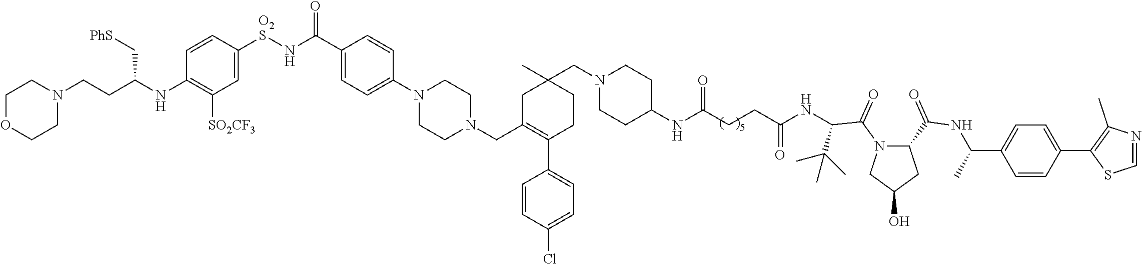 | +++ | ++++ |
| 39 | 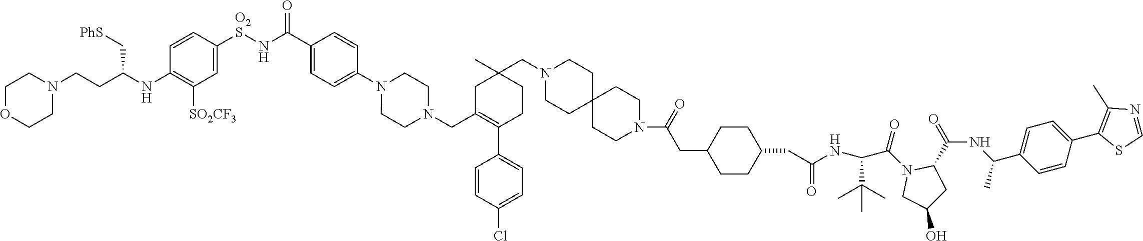 | +++ | ++++ |
| 40 | 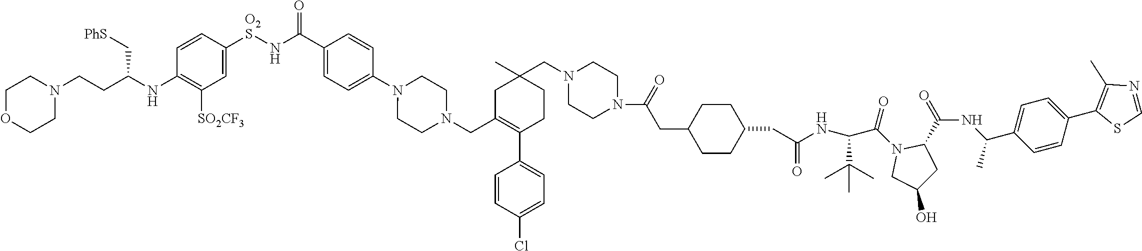 | ++++ | ++++ |
| 41 | 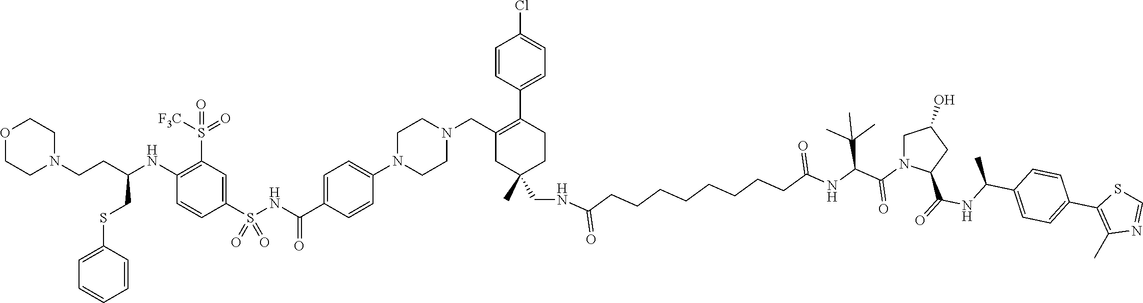 [SMILES: Cc1ncsc1-c1ccc([C@H](C)NC(=O)[C@@H]2C[C@@H](O)CN2C(=O)[C@@H](NC(=O)CCCCCCCCC(=O)NC[C@@]2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ++ | + |
| 42 | 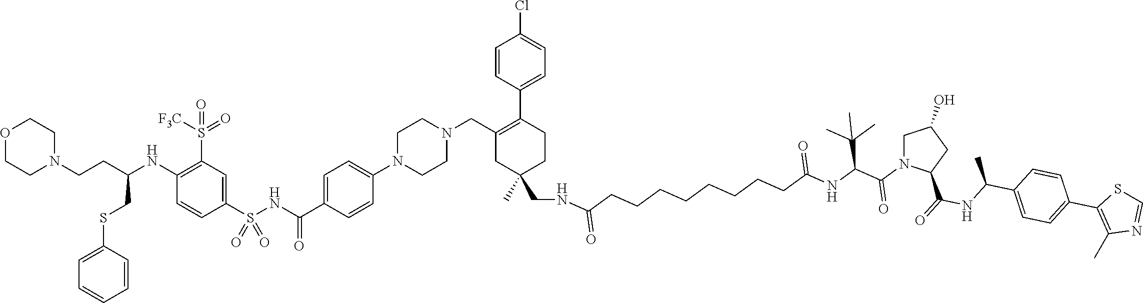 [SMILES: Cc1ncsc1-c1ccc([C@H](C)NC(=O)[C@@H]2C[C@@H](O)CN2C(=O)[C@@H](NC(=O)CCCCCCCCC(=O)NC[C@]2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ++++ | ++++ |
| 43 | 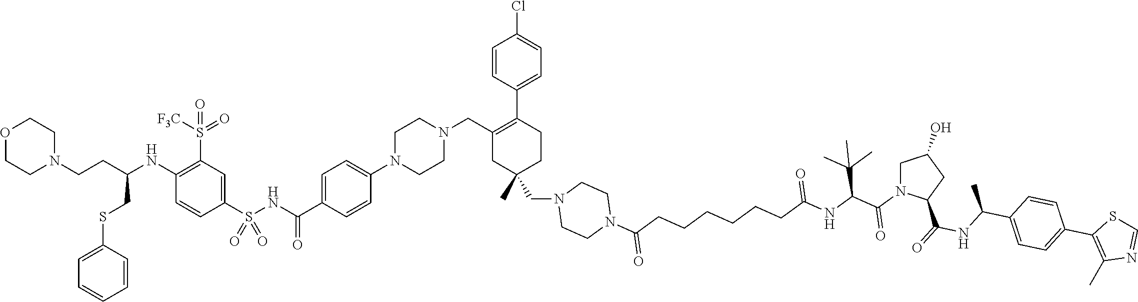 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | +++ | ++ |
| 44 | 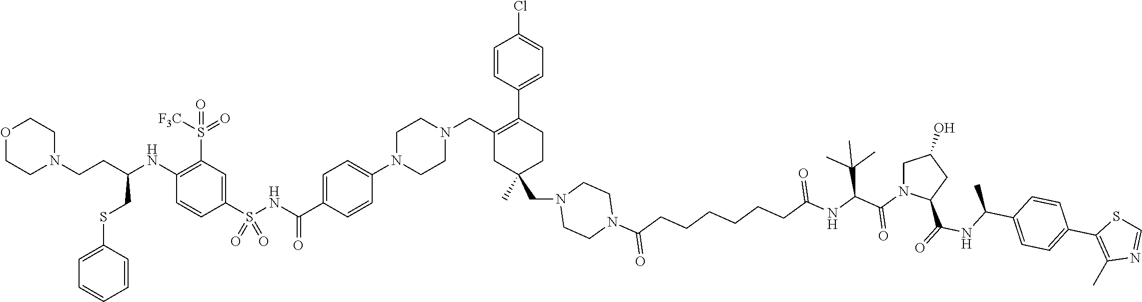 [SMILES: Cc1ncsc1-c1ccc([C@H](C)NC(=O)[C@@H]2C[C@@H](O)CN2C(=O)[C@@H](NC(=O)CCCCCCC(=O)N2CCN(C[C@]3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | ++++ | ++++ |
| 45 | 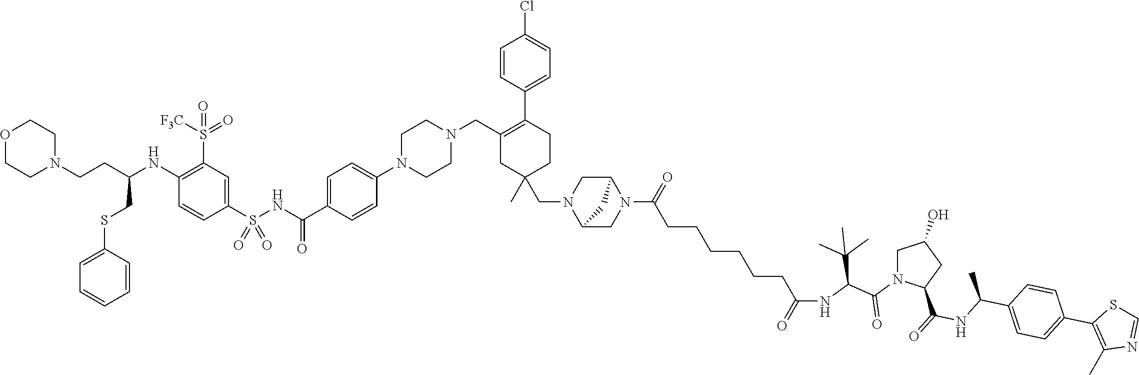 [SMILES: Cc1ncsc1-c1ccc([C@H](C)NC(=O)[C@@H]2C[C@@H](O)CN2C(=O)[C@@H](NC(=O)CCCCCCC(=O)N2C[C@@H]3C[C@H]2CN3CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | +++ | ++++ |
| 46 | 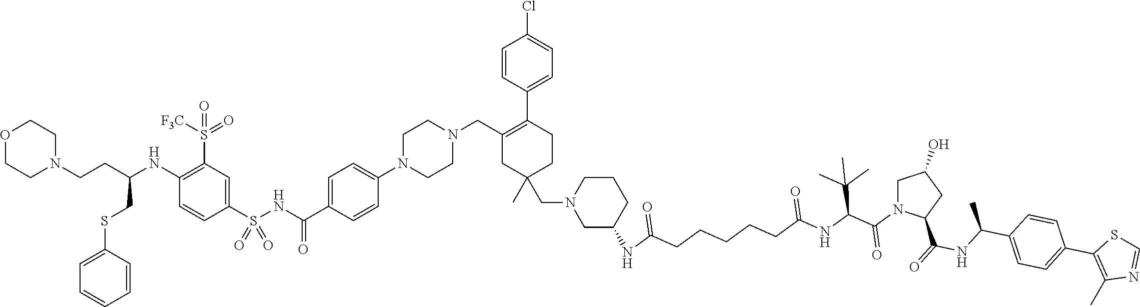 [SMILES: Cc1ncsc1-c1ccc([C@H](C)NC(=O)[C@@H]2C[C@@H](O)CN2C(=O)[C@@H](NC(=O)CCCCCC(=O)N[C@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1] | ++ | +++ |
| 47 | 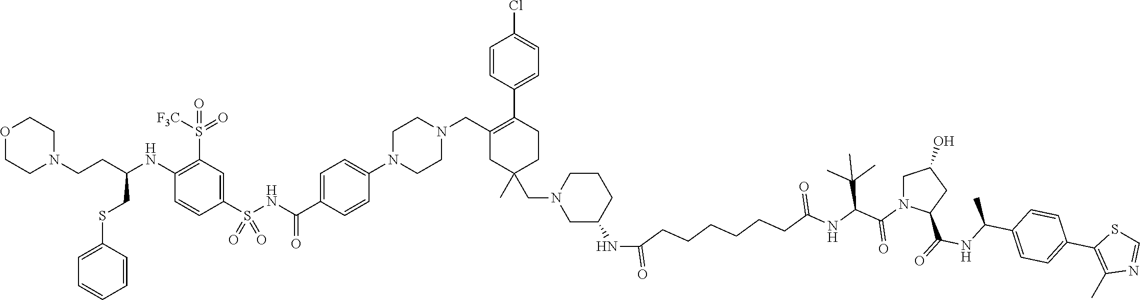 [SMILES: Cc1ncsc1-c1ccc([C@H](C)NC(=O)[C@@H]2C[C@@H](O)CN2C(=O)[C@@H](NC(=O)CCCCCCC(=O)N[C@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1] | +++ | +++ |
| 48 | 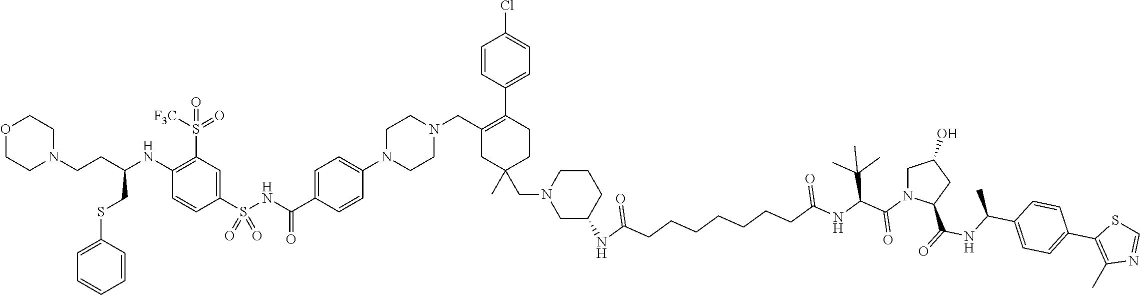 [SMILES: Cc1ncsc1-c1ccc([C@H](C)NC(=O)[C@@H]2C[C@@H](O)CN2C(=O)[C@@H](NC(=O)CCCCCCCC(=O)N[C@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1] | +++ | ++++ |
| 49 | 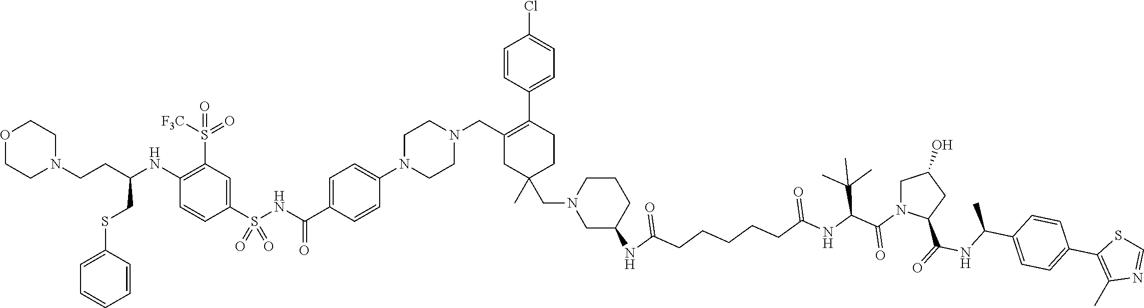 [SMILES: Cc1ncsc1-c1ccc([C@H](C)NC(=O)[C@@H]2C[C@@H](O)CN2C(=O)[C@@H](NC(=O)CCCCCC(=O)N[C@@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1] | ++ | +++ |
| 50 | 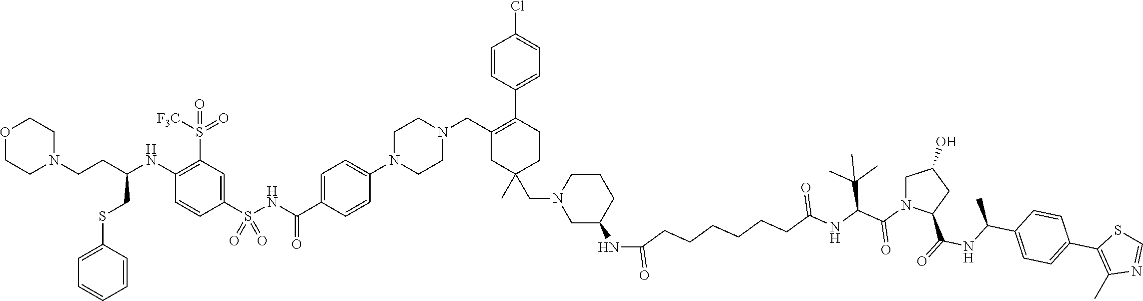 [SMILES: Cc1ncsc1-c1ccc([C@H](C)NC(=O)[C@@H]2C[C@@H](O)CN2C(=O)[C@@H](NC(=O)CCCCCCC(=O)N[C@@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1] | +++ | ++++ |
| 51 | 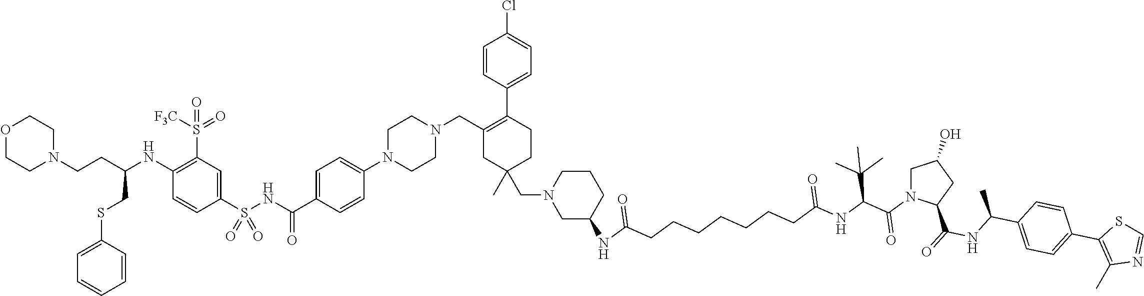 [SMILES: Cc1ncsc1-c1ccc([C@H](C)NC(=O)[C@@H]2C[C@@H](O)CN2C(=O)[C@@H](NC(=O)CCCCCCCC(=O)N[C@@H]2CCCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)C2)C(C)(C)C)cc1] | +++ | ++++ |
| 52 | 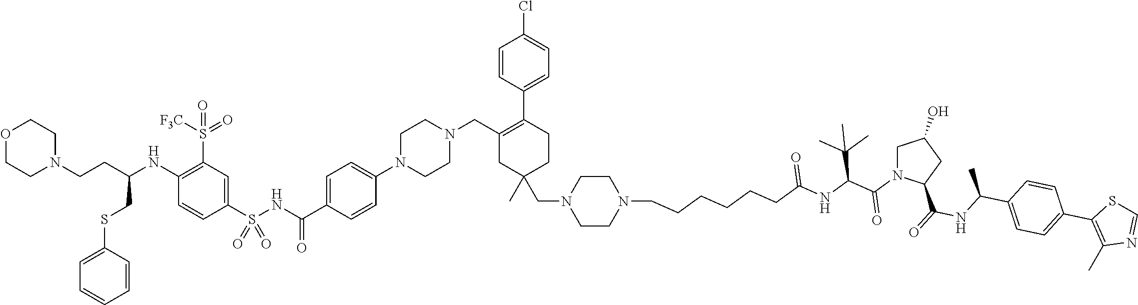 [SMILES: Cc1ncsc1-c1ccc([C@H](C)NC(=O)[C@@H]2C[C@@H](O)CN2C(=O)[C@@H](NC(=O)CCCCCCN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | +++ | +++ |
| 53 | 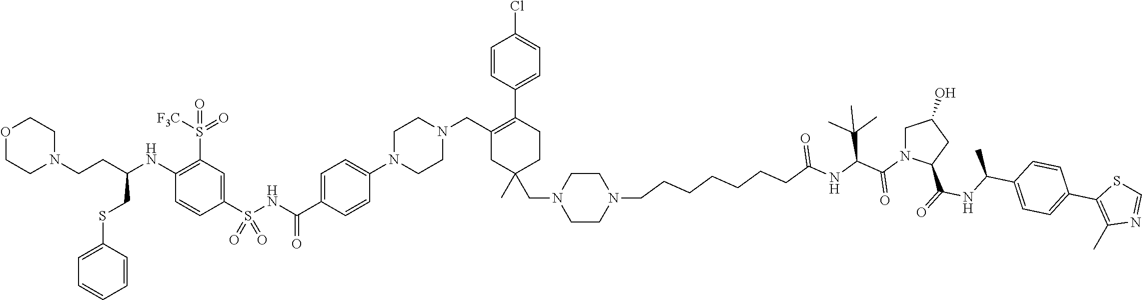 [SMILES: Cc1ncsc1-c1ccc([C@H](C)NC(=O)[C@@H]2C[C@@H](O)CN2C(=O)[C@@H](NC(=O)CCCCCCCN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | +++ | +++ |
| 54 | 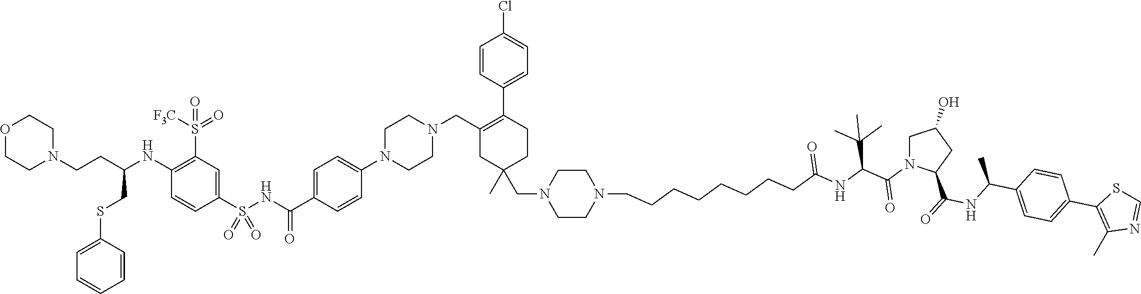 [SMILES: Cc1ncsc1-c1ccc([C@H](C)NC(=O)[C@@H]2C[C@@H](O)CN2C(=O)[C@@H](NC(=O)CCCCCCCCN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | +++ | ++ |
| 55 | 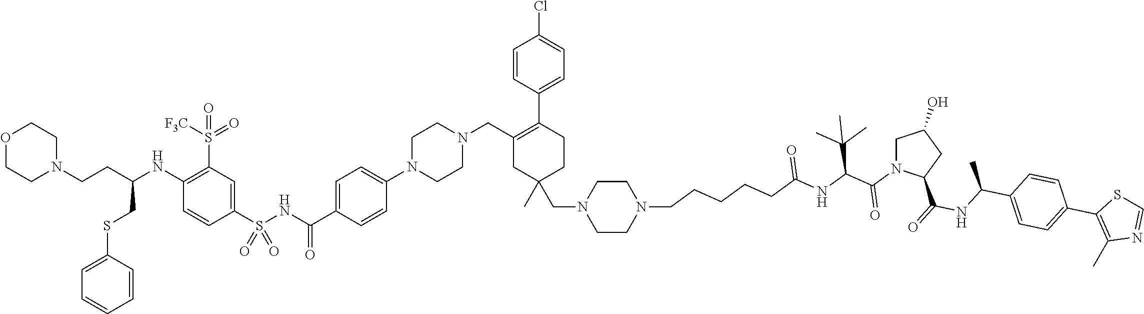 [SMILES: Cc1ncsc1-c1ccc([C@H](C)NC(=O)[C@@H]2C[C@@H](O)CN2C(=O)[C@@H](NC(=O)CCCCCN2CCN(CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | ++++ | ++++ |
| 56 | 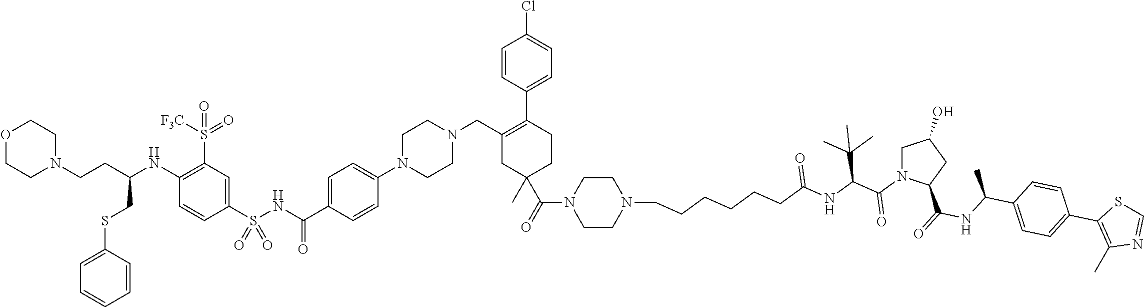 [SMILES: Cc1ncsc1-c1ccc([C@H](C)NC(=O)[C@@H]2C[C@@H](O)CN2C(=O)[C@@H](NC(=O)CCCCCCN2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | ++++ | ++++ |
| 57 | 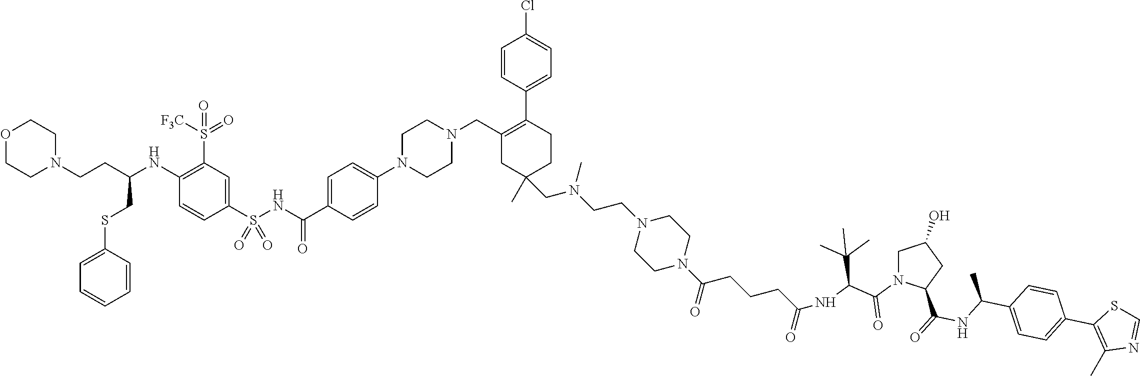 [SMILES: Cc1ncsc1-c1ccc([C@H](C)NC(=O)[C@@H]2C[C@@H](O)CN2C(=O)[C@@H](NC(=O)CCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | ++ | ++ |
| 58 | 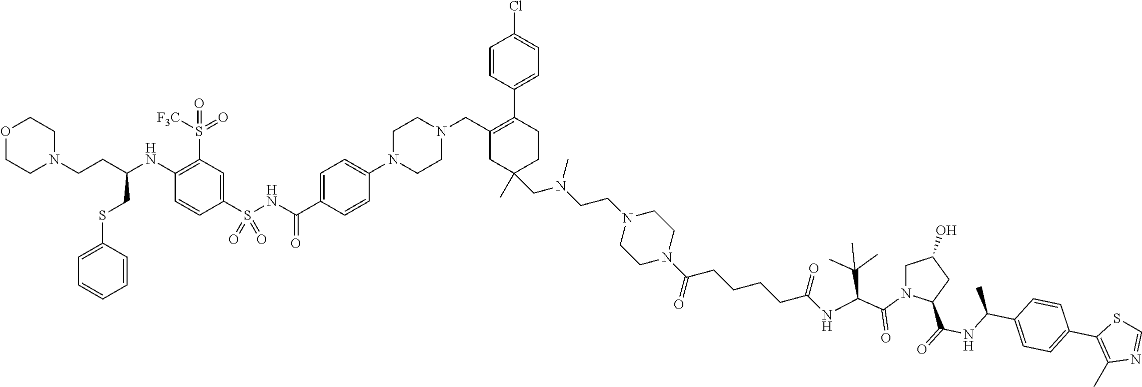 [SMILES: Cc1ncsc1-c1ccc([C@H](C)NC(=O)[C@@H]2C[C@@H](O)CN2C(=O)[C@@H](NC(=O)CCCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | +++ | +++ |
| 59 | 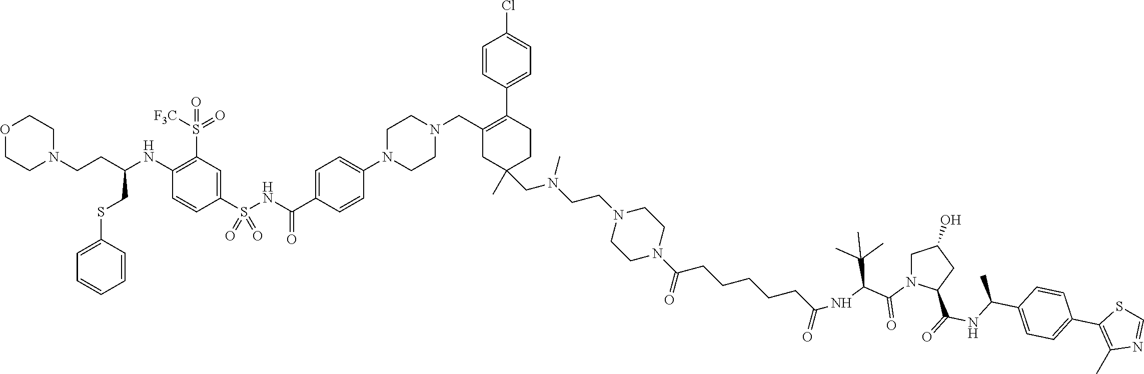 [SMILES: Cc1ncsc1-c1ccc([C@H](C)NC(=O)[C@@H]2C[C@@H](O)CN2C(=O)[C@@H](NC(=O)CCCCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | ++++ | ++++ |
| 60 | 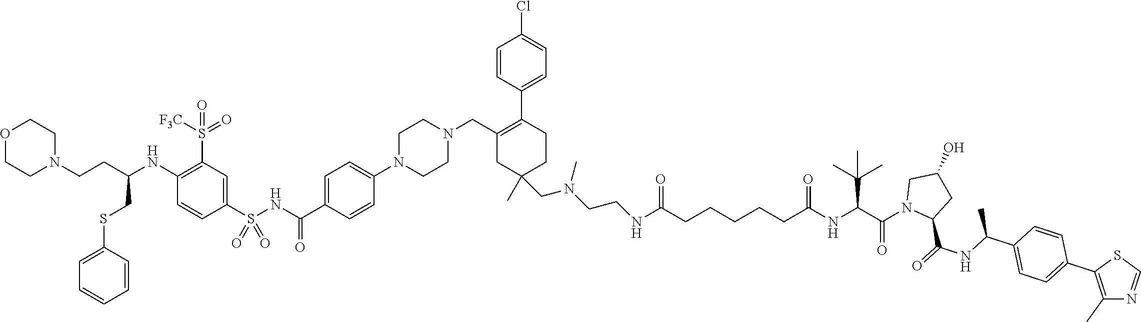 [SMILES: Cc1ncsc1-c1ccc([C@H](C)NC(=O)[C@@H]2C[C@@H](O)CN2C(=O)[C@@H](NC(=O)CCCCCC(=O)NCCN(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ++ | ++ |
| 61 | 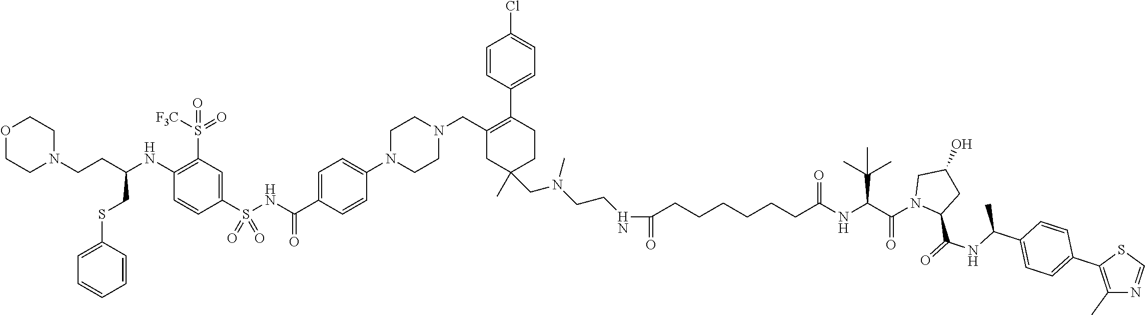 [SMILES: Cc1ncsc1-c1ccc([C@H](C)NC(=O)[C@@H]2C[C@@H](O)CN2C(=O)[C@@H](NC(=O)CCCCCCC(=O)NCCN(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ++ | +++ |
| 62 | 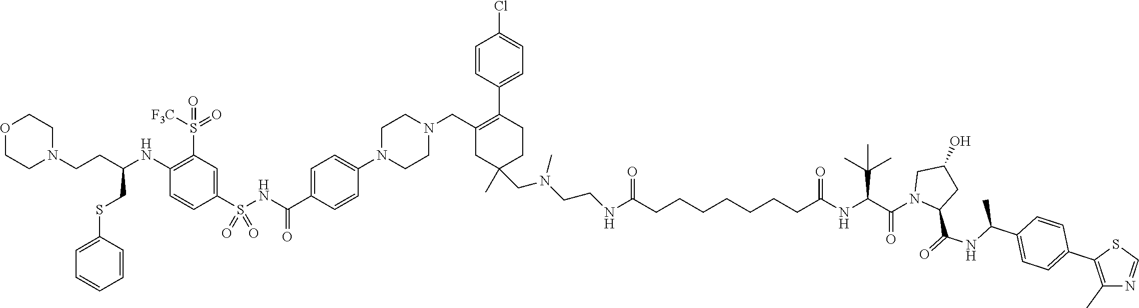 [SMILES: Cc1ncsc1-c1ccc([C@H](C)NC(=O)[C@@H]2C[C@@H](O)CN2C(=O)[C@@H](NC(=O)CCCCCCCC(=O)NCCN(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ++ | ++++ |
| 63 | 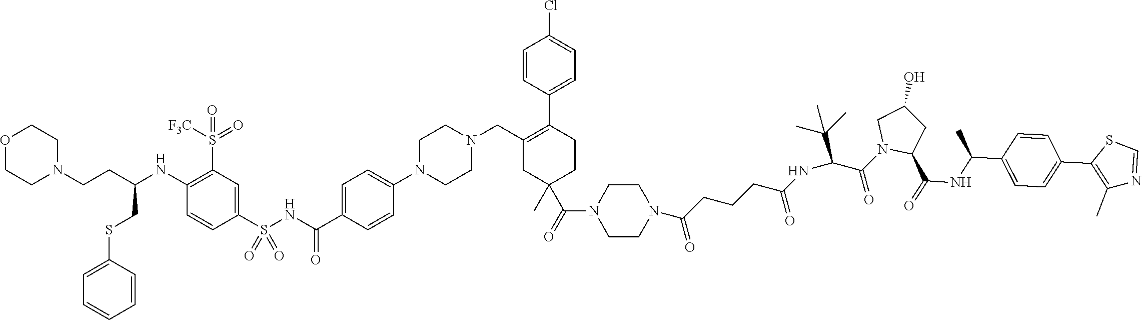 [SMILES: Cc1ncsc1-c1ccc([C@H](C)NC(=O)[C@@H]2C[C@@H](O)CN2C(=O)[C@@H](NC(=O)CCCC(=O)N2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | + | + |
| 64 | 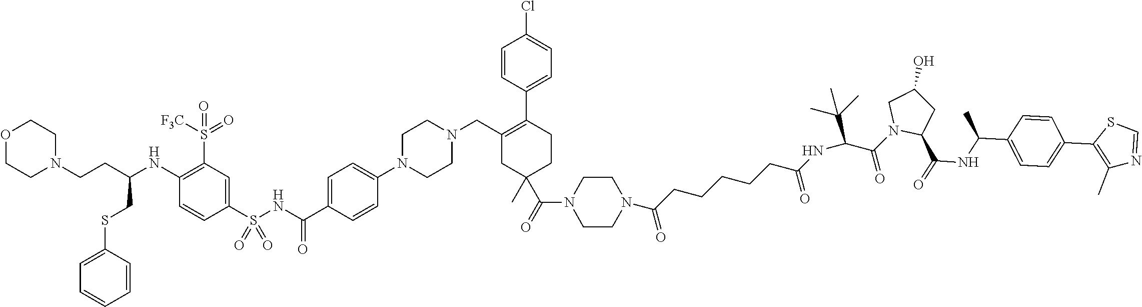 [SMILES: Cc1ncsc1-c1ccc([C@H](C)NC(=O)[C@@H]2C[C@@H](O)CN2C(=O)[C@@H](NC(=O)CCCCCC(=O)N2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | +++ | +++ |
| 65 | 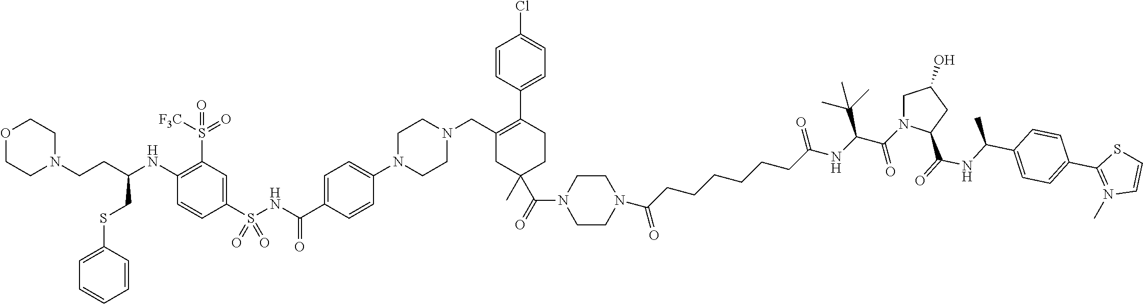 | ++++ | ++++ |
| 66 | 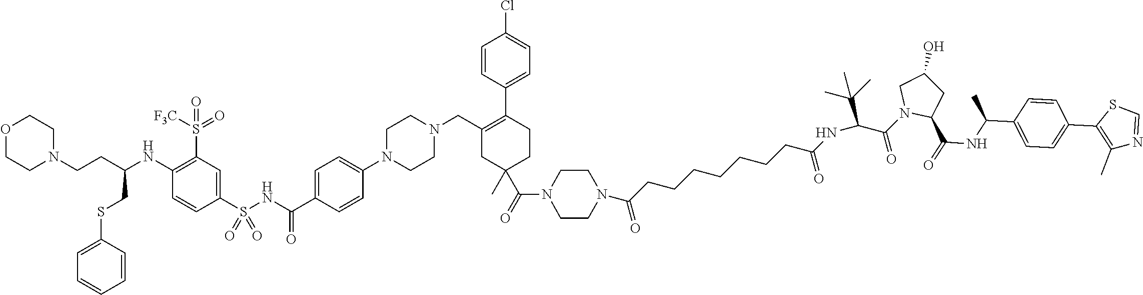 [SMILES: Cc1ncsc1-c1ccc([C@H](C)NC(=O)[C@@H]2C[C@@H](O)CN2C(=O)[C@@H](NC(=O)CCCCCCCC(=O)N2CCN(C(=O)C3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | ++++ | ++++ |
| 67 | 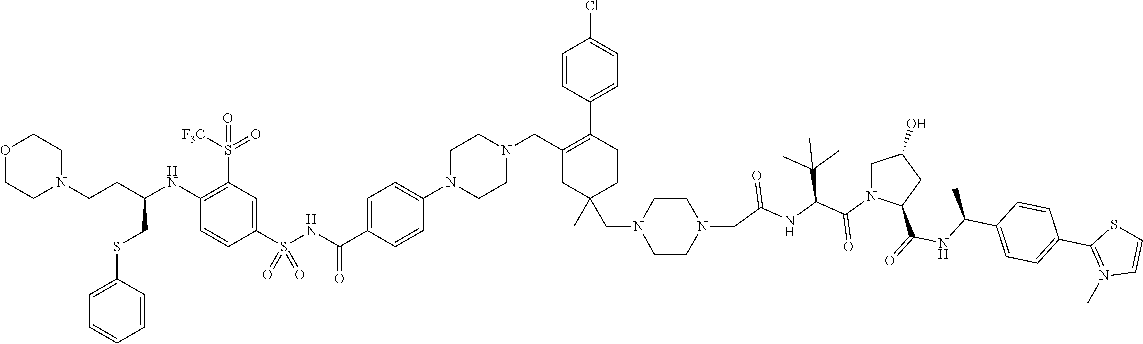 | +++ | +++ |
| 68 | 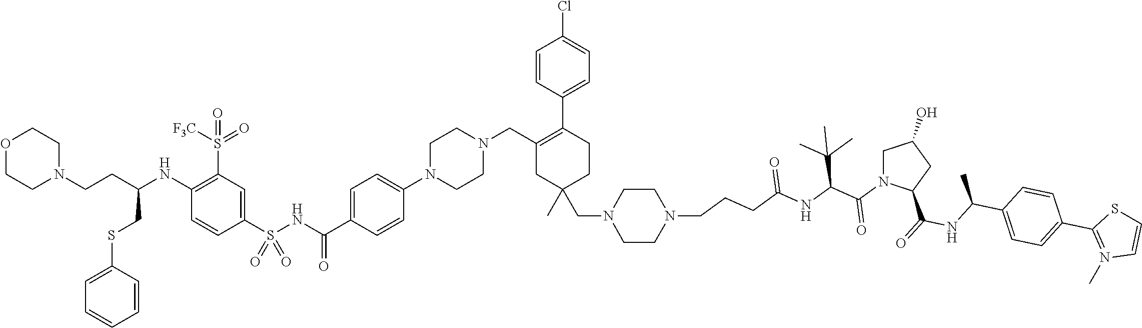 | + | ++ |
| 69 | 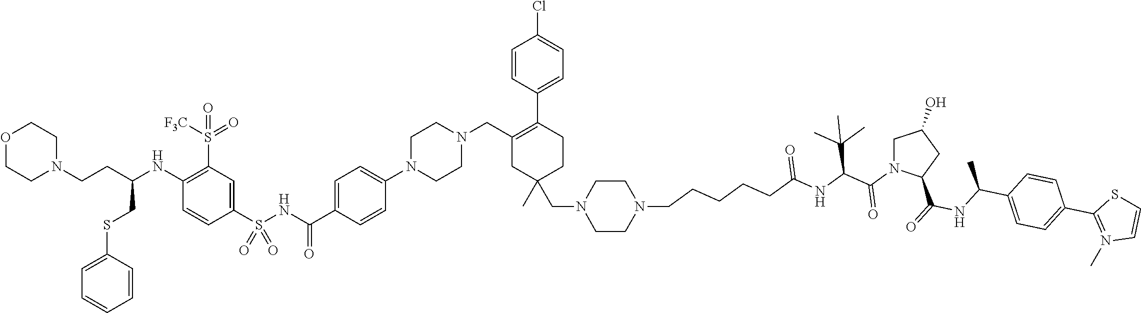 | +++ | +++ |
| 70 | 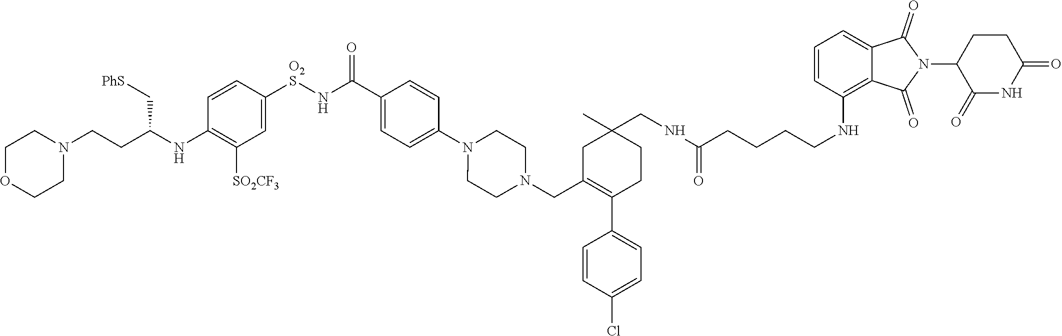 | +++ | +++ |
| 71 | 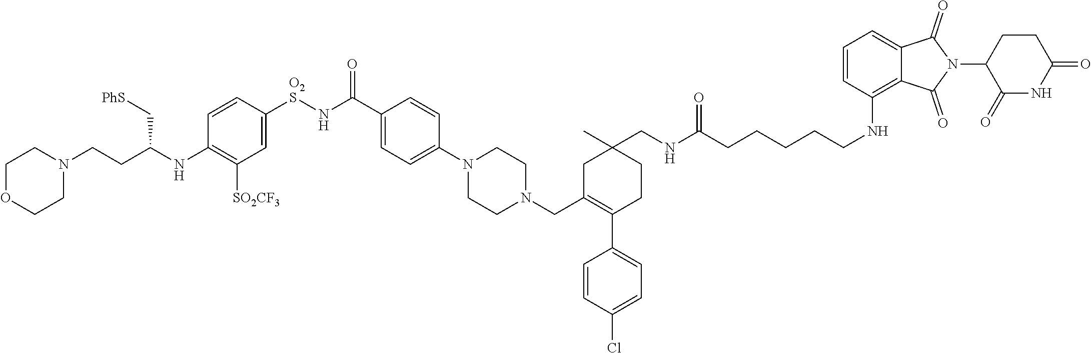 | ++++ | ++++ |
| 72 | 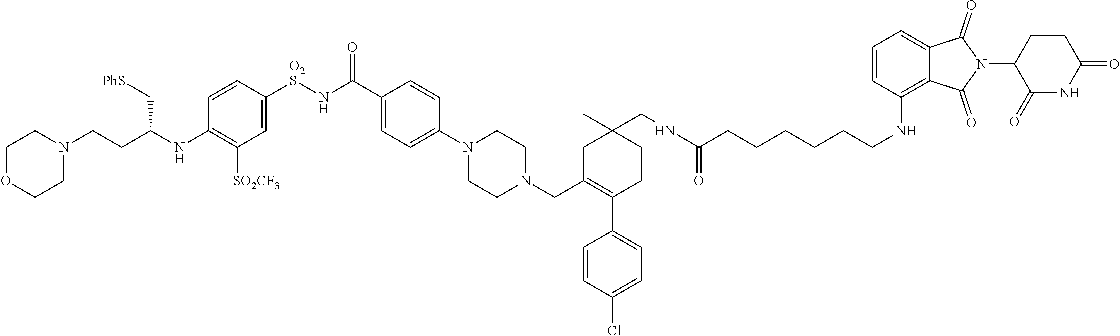 | ++++ | ++++ |
| 73 | 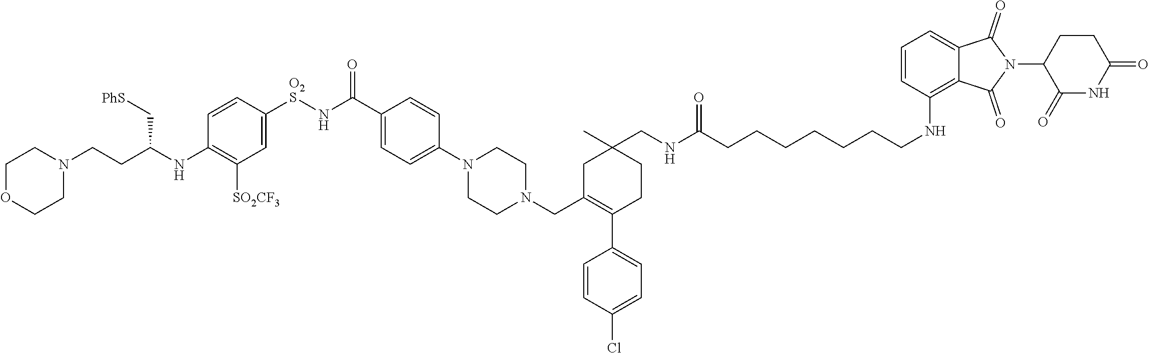 | ++++ | ++++ |
| 74 | 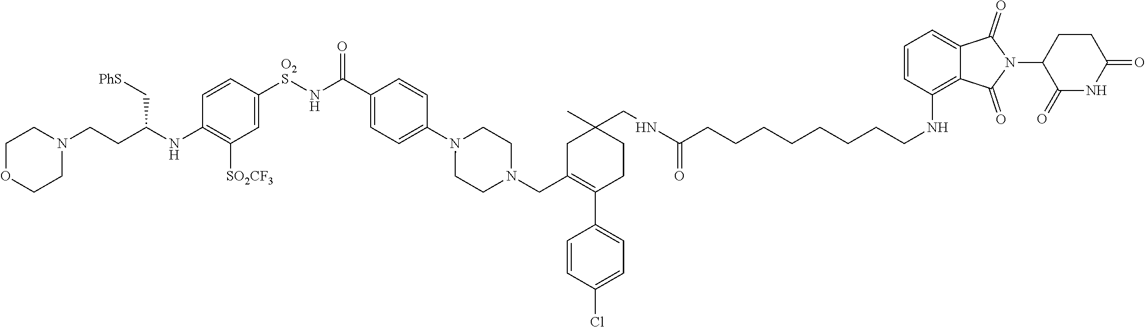 | ++++ | +++ |
| 75 | 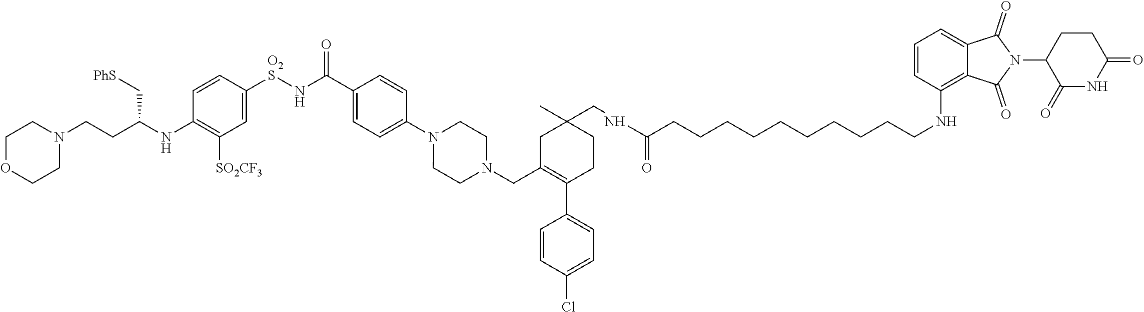 | +++ | ++ |
| 76 | 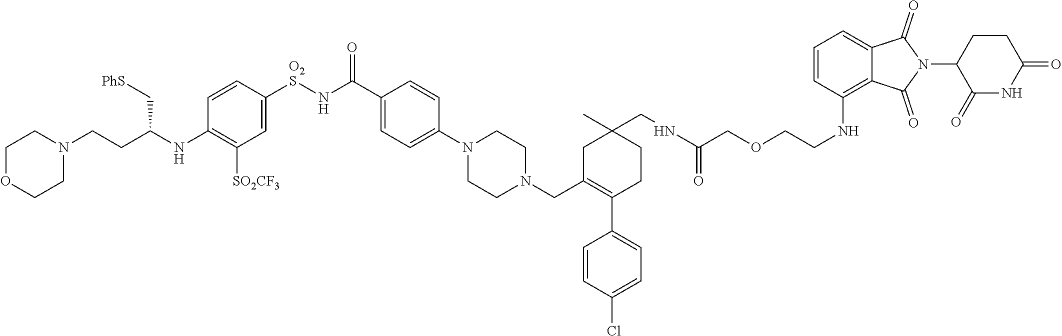 | +++ | +++ |
| 77 | 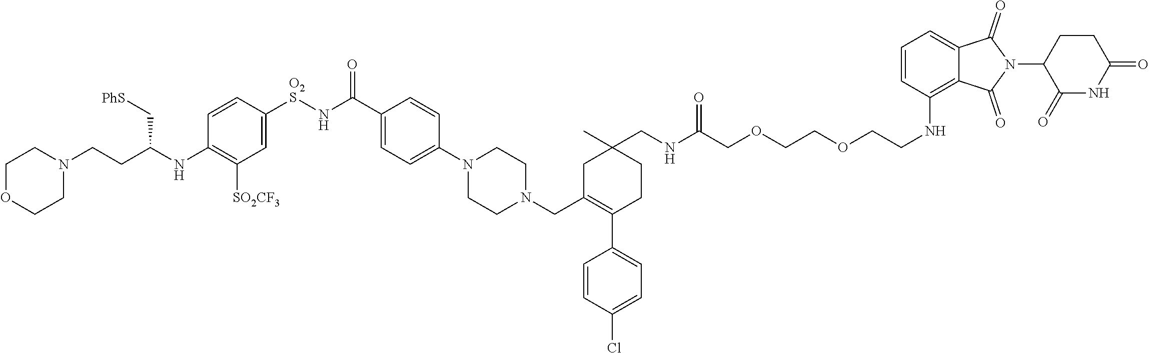 | ++++ | ++++ |
| 78 | 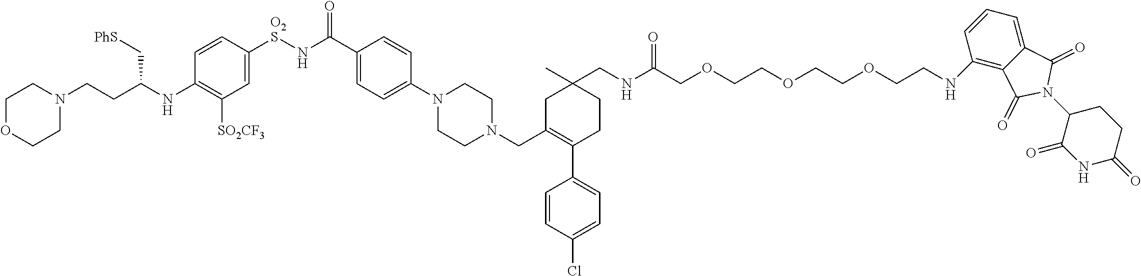 | ++++ | ++++ |
| 79 | 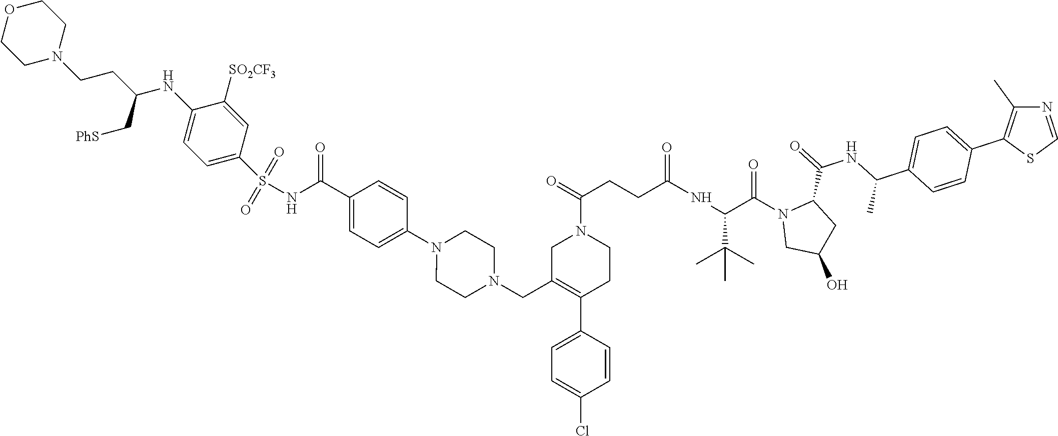 | + | + |
| 80 | 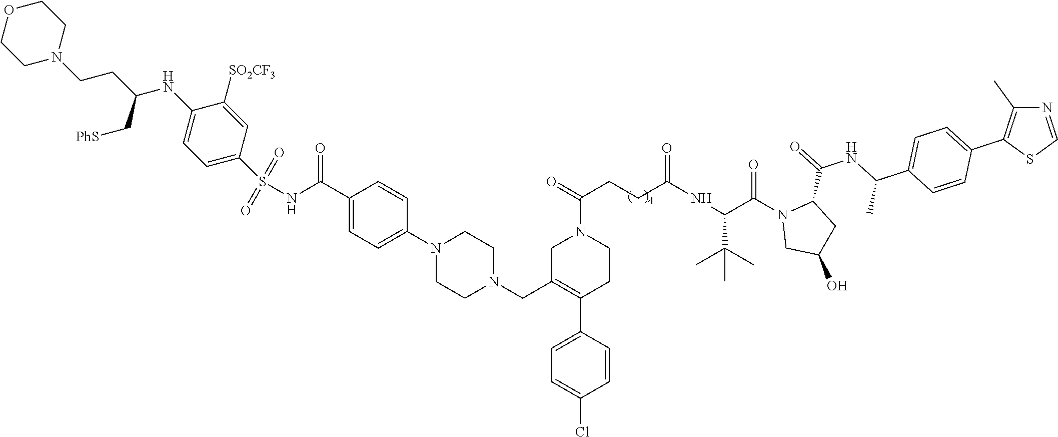 | + | + |
| 81 | 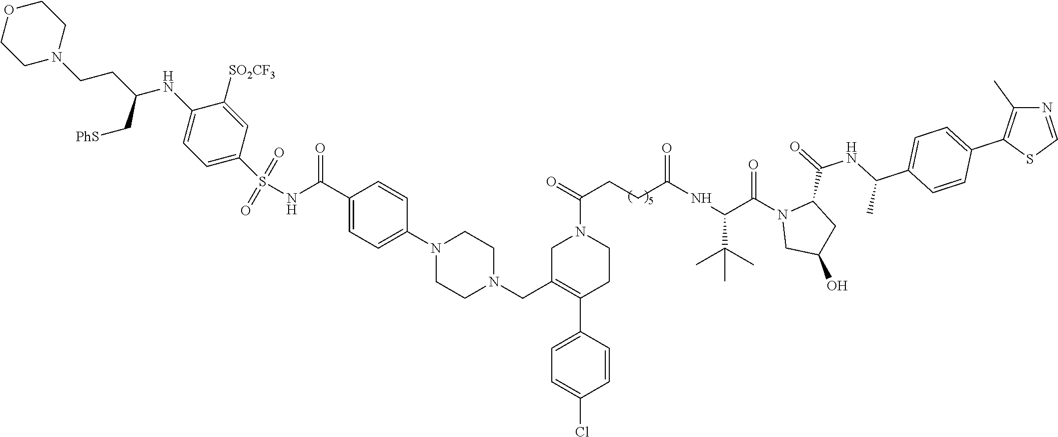 | ++ | ++ |
| 82 | 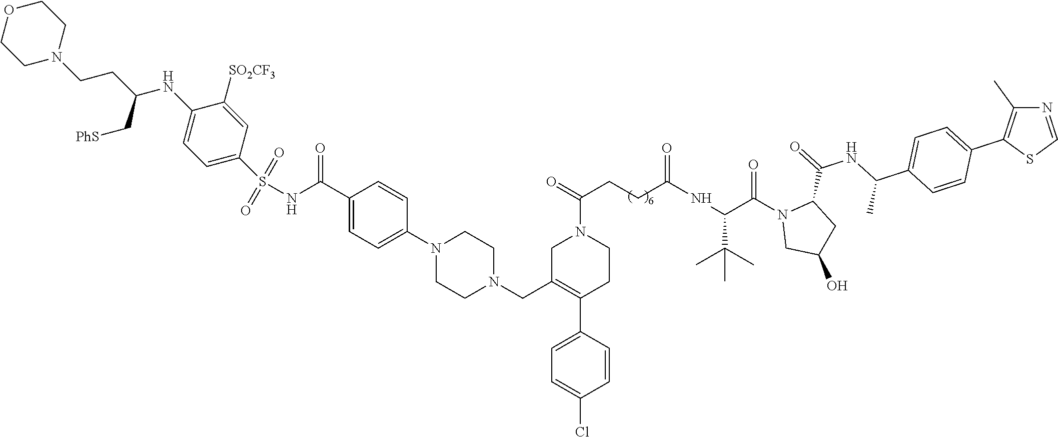 | + | + |
| 83 | 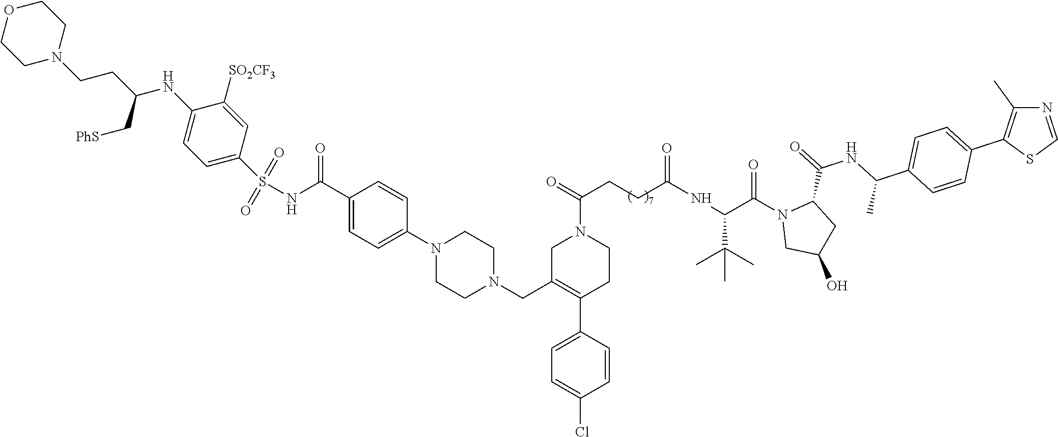 | +++ | ++ |
| 84 | 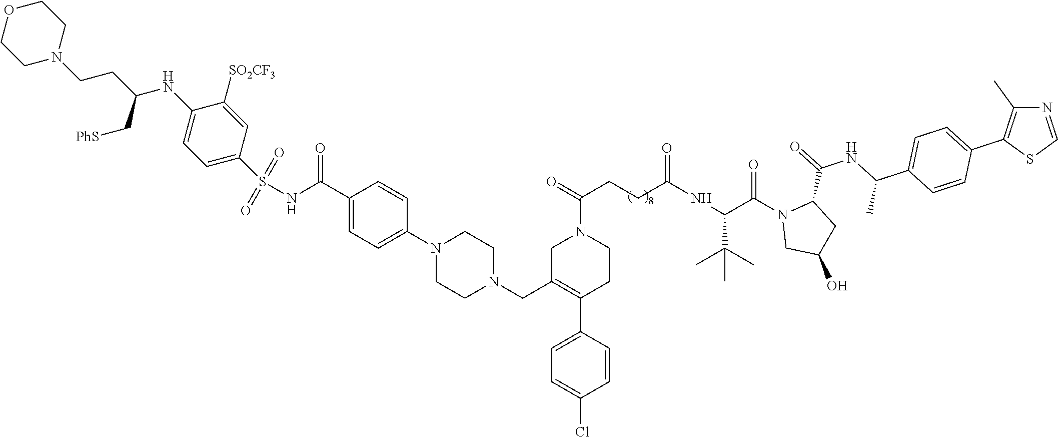 | ++ | ++ |
| 85 | 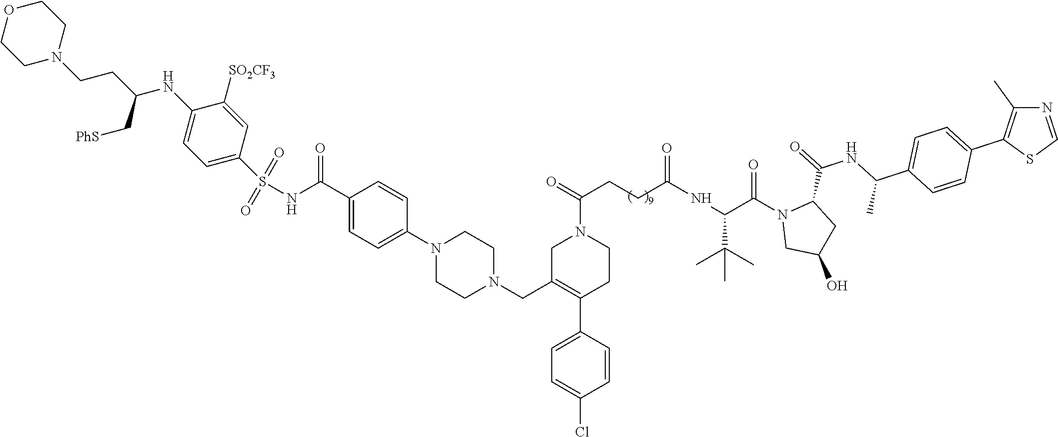 | +++ | +++ |
| 86 | 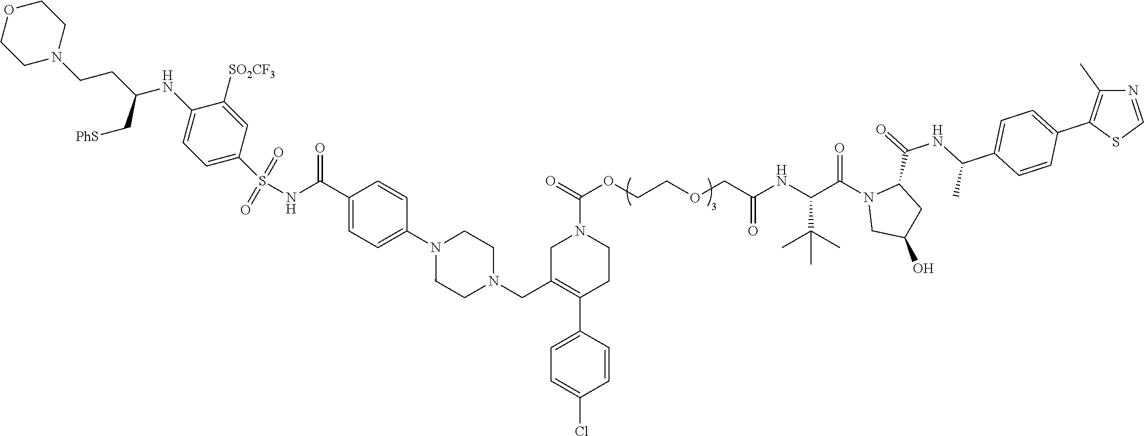 | + | + |
| 87 | 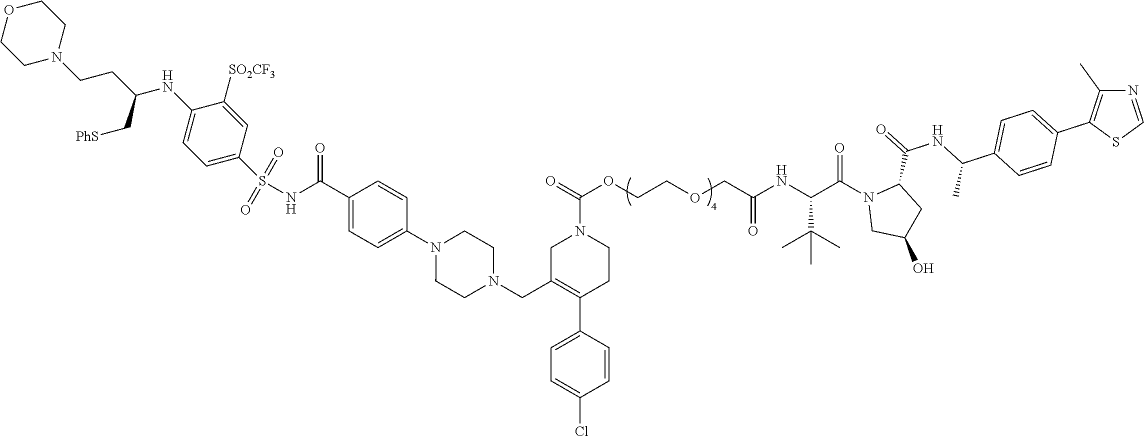 | + | + |
| 88 | 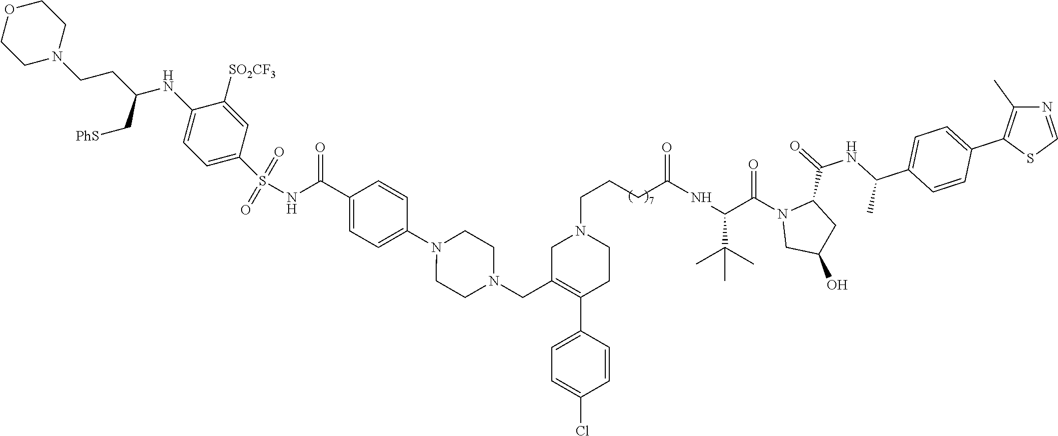 | ++ | + |
| 89 | 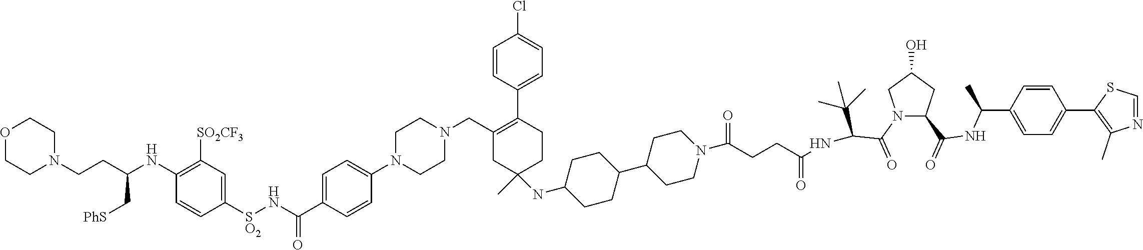 | ND | ND |
| 90 | 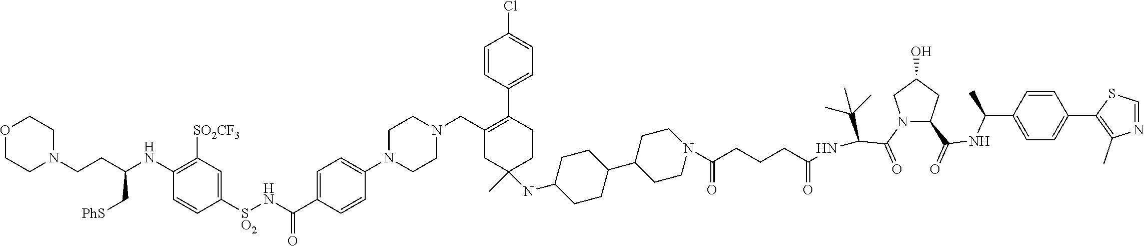 | ND | ND |
| 91 | 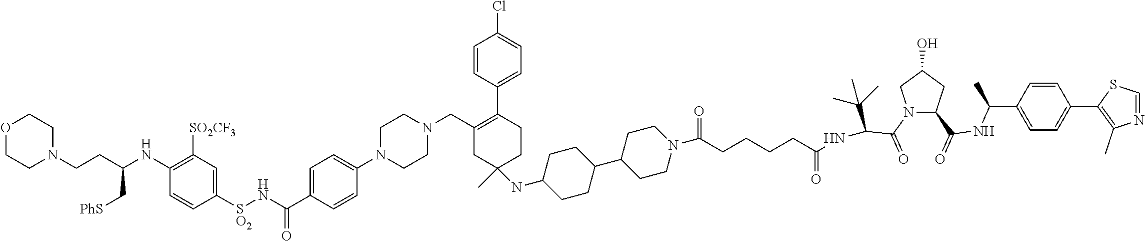 | ND | ND |
| 92 | 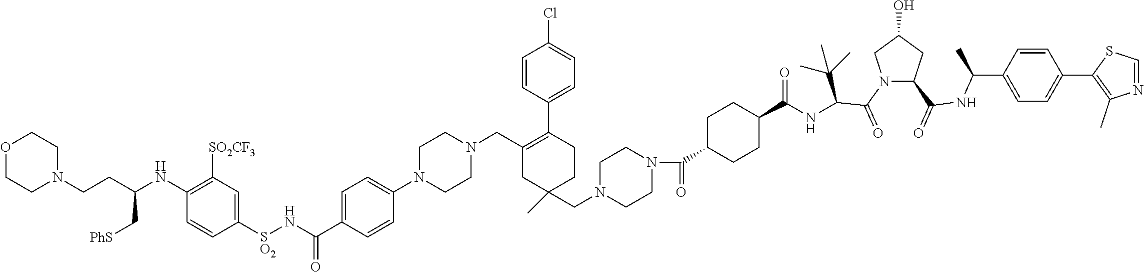 | ND | ND |
| 93 | 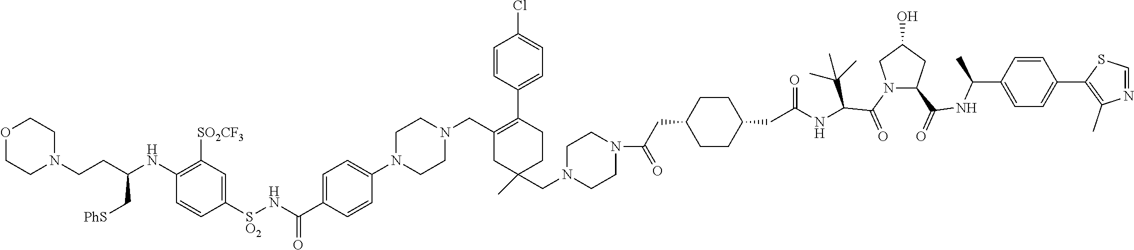 | ND | ND |
| 94 | 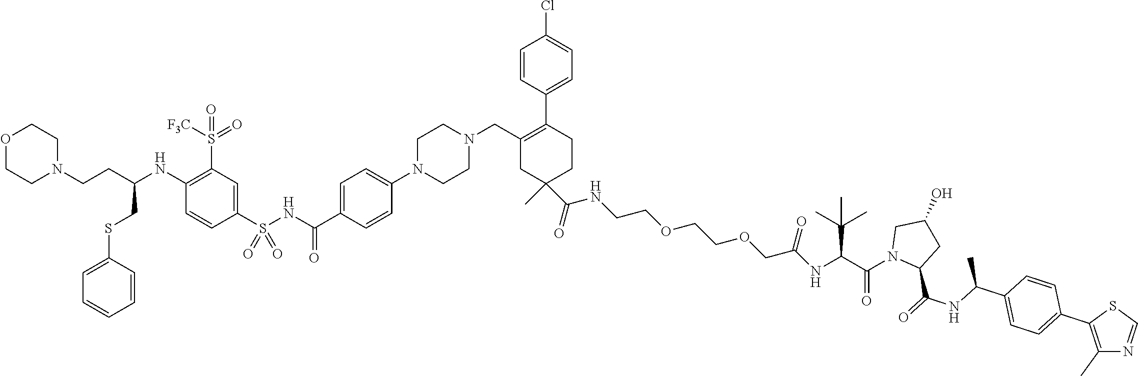 [SMILES: Cc1ncsc1-c1ccc([C@H](C)NC(=O)[C@@H]2C[C@@H](O)CN2C(=O)[C@@H](NC(=O)COCCOCCNC(=O)C2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ND | ND |
| 95 | 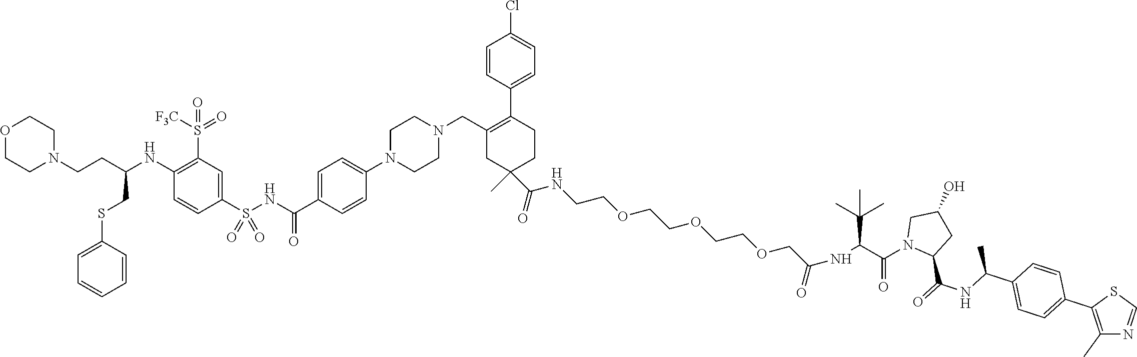 [SMILES: Cc1ncsc1-c1ccc([C@H](C)NC(=O)[C@@H]2C[C@@H](O)CN2C(=O)[C@@H](NC(=O)COCCOCCOCCNC(=O)C2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ND | ND |
| 96 | 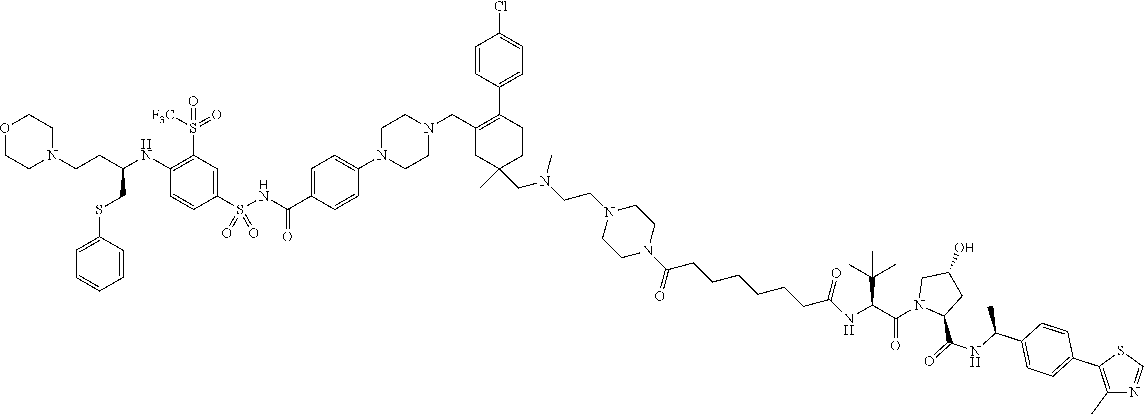 [SMILES: Cc1ncsc1-c1ccc([C@H](C)NC(=O)[C@@H]2C[C@@H](O)CN2C(=O)[C@@H](NC(=O)CCCCCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | ND | ND |
| 97 | 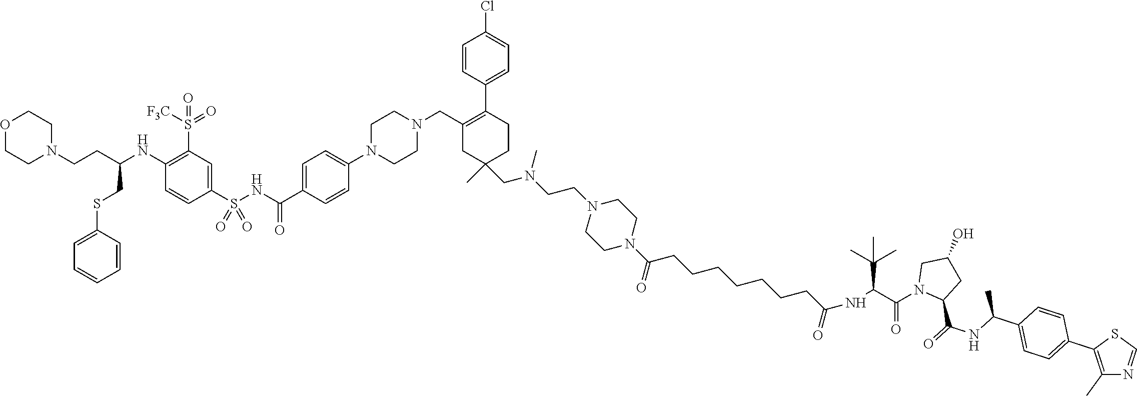 [SMILES: Cc1ncsc1-c1ccc([C@H](C)NC(=O)[C@@H]2C[C@@H](O)CN2C(=O)[C@@H](NC(=O)CCCCCCCC(=O)N2CCN(CCN(C)CC3(C)CCC(c4ccc(Cl)cc4)=C(CN4CCN(c5ccc(C(=O)NS(=O)(=O)c6ccc(N[C@H](CCN7CCOCC7)CSc7ccccc7)c(S(=O)(=O)C(F)(F)F)c6)cc5)CC4)C3)CC2)C(C)(C)C)cc1] | ND | ND |
| 98 | 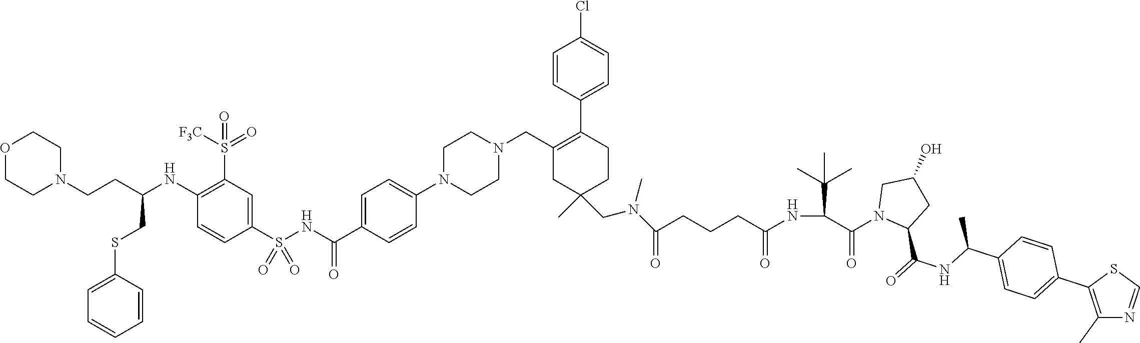 [SMILES: Cc1ncsc1-c1ccc([C@H](C)NC(=O)[C@@H]2C[C@@H](O)CN2C(=O)[C@@H](NC(=O)CCCC(=O)N(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ND | ND |
| 99 | 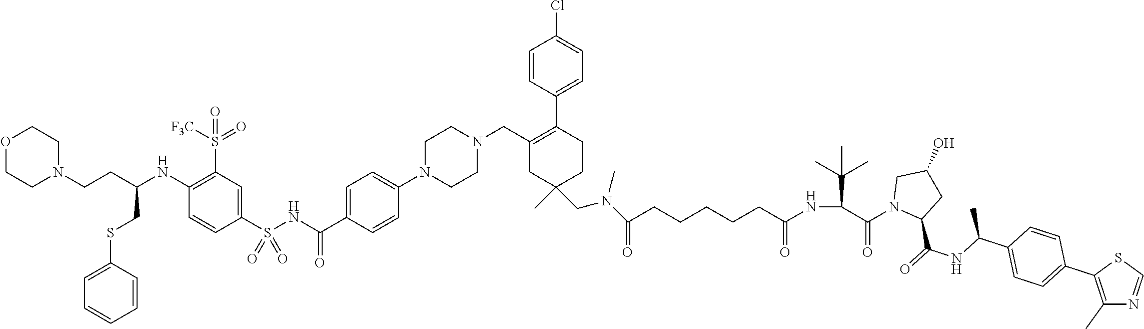 [SMILES: Cc1ncsc1-c1ccc([C@H](C)NC(=O)[C@@H]2C[C@@H](O)CN2C(=O)[C@@H](NC(=O)CCCCCC(=O)N(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ND | ND |
| 100 | 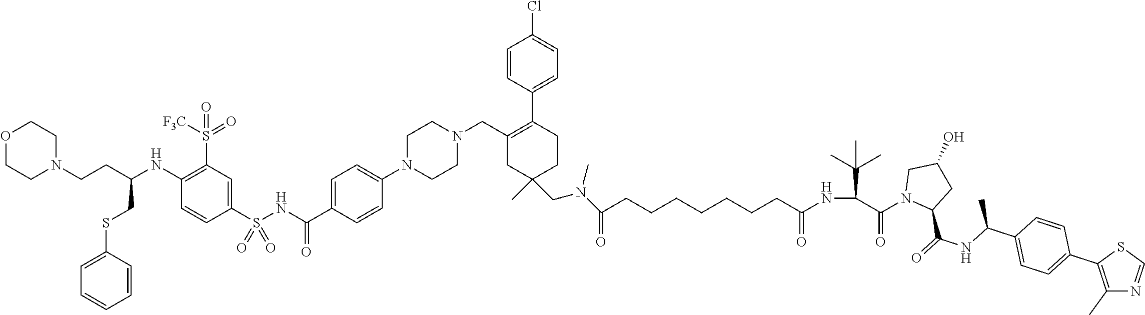 [SMILES: Cc1ncsc1-c1ccc([C@H](C)NC(=O)[C@@H]2C[C@@H](O)CN2C(=O)[C@@H](NC(=O)CCCCCCCC(=O)N(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ND | ND |
| 101 | 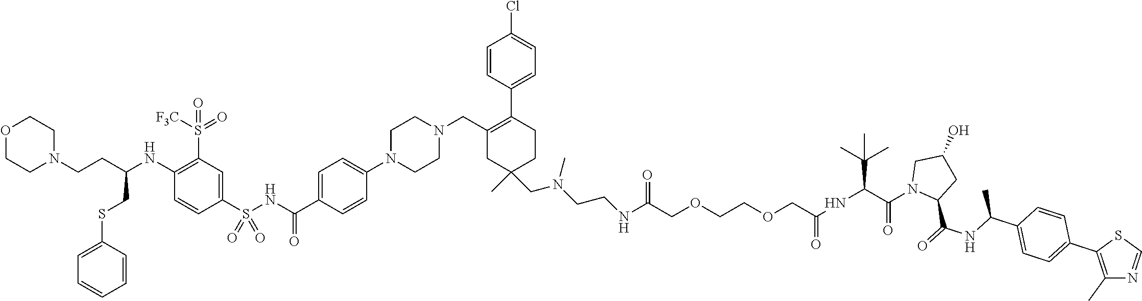 [SMILES: Cc1ncsc1-c1ccc([C@H](C)NC(=O)[C@@H]2C[C@@H](O)CN2C(=O)[C@@H](NC(=O)COCCOCC(=O)NCCN(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1] | ND | ND |

^{a}<50 nM = ++++, 50-150 nM = +++, 150-500 nM = ++, >500 nM = +, ND = not determined

### Example 24: Preparation of Degraders #1-10

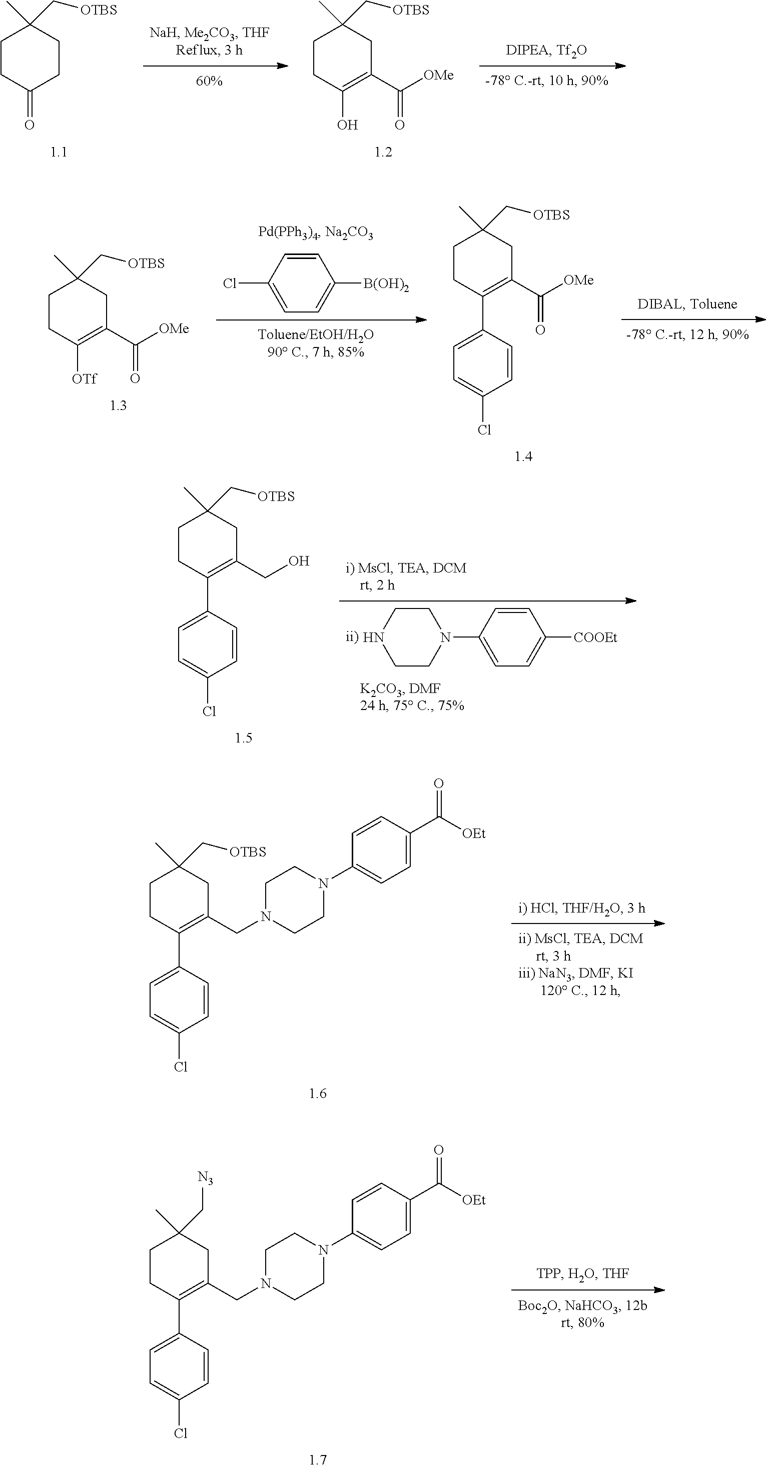

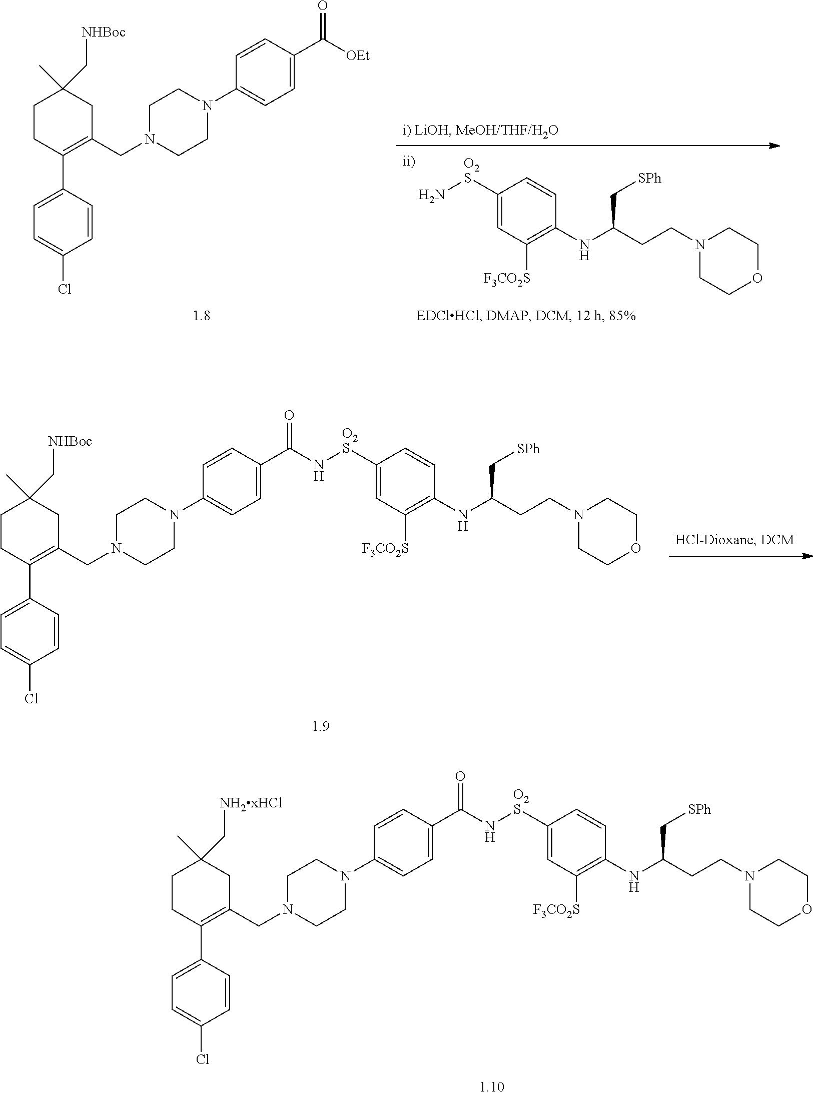

Preparation of methyl 5-((tert-butyldimethylsilyloxy)methyl)-2-hydroxy-5-methylcyclohex-1-enecarboxylate (1.2): To a solution of compound 1.1 (7.05 g, 27.54 mmol) in THF (90 mL) at 0° C. was added NaH (3.3 g, 82.62 mmol) portionwise and the mixture was stirred at the same temperature for 1 h. Neat Me₂CO₃ (7.4 g, 82.62 mmol) was added to the mixture and the solution was heated under reflux for 3 h. The reaction was quenched with saturated NH₄Cl solution at 0° C. and THE was removed under reduced pressure. The residue was diluted with EtOAc and washed with water and brine. The organic portion was dried over anhydrous Na₂SO₄, filtered, and solvent was removed under reduced pressure. The crude material was purified by flash chromatography (Hexane/EtOAc=10:1) to afford the title compound (5.1 g, 16.5 mmol, 60% yield). ¹H NMR (600 MHz, CDCl₃) δ 12.15 (s, 1H), 3.75 (s, 3H), 3.35 (d, J=9.6 Hz, 1H), 3.28 (d, J=9.5 Hz, 1H), 2.28 (ddd, J=7.4, 5.4, 1.3 Hz, 2H), 2.12 (dt, J=15.9, 1.7 Hz, 1H), 1.94-1.90 (m, 1H), 1.62 (dt, J=13.2, 7.4 Hz, 1H), 1.40 (ddt, J=13.4, 6.1, 1.3 Hz, 1H), 0.90 (s, 3H), 0.89 (s, 9H), 0.02 (d, J=1.4 Hz, 6H) ppm. ESI⁺, m/z [M+H]⁺=315.2.

Preparation of methyl 5-((tert-butyldimethylsilyloxy)methyl)-5-methyl-2-(trifluoromethylsulfonyloxy)cyclohex-1-enecarboxylate (1.3): To a stirring solution of compound 1.2 (5.1 g, 16.5 mmol) in DCM (65 mL) was added DIPEA (14.5 mL, 82.5 mmol) at −78° C. and the mixture was stirred for 0.5 h at the same temperature. Tf₂O (4.2 ml, 24.75 mmol) was added to the reaction mixture and stirred for 10 h at room temperature. The reaction was diluted with DCM (100 mL) and quenched with water. The organic portion was washed with dilute HCl followed by brine. The organic portion was dried over anhydrous Na₂SO₄, filtered, and solvent was removed under reduced pressure. The crude material was purified by flash chromatography (Hexanes/EtOAc=10:1) to afford the title compound (6.6 g, 14.8 mmol, 90% yield). ¹H NMR (600 MHz, CDCl₃) δ 3.80 (s, 3H), 3.37 (d, J=9.7 Hz, 1H), 3.30 (d, J=9.6 Hz, 1H), 2.41 (ddd, J=15.4, 7.2, 3.0 Hz, 3H), 2.20-2.15 (m, 1H), 1.77-1.71 (m, 1H), 1.47 (ddd, J=11.9, 8.2, 5.3 Hz, 1H), 0.93 (s, 3H), 0.89 (s, 9H), 0.03 (s, 6H) ppm.

Preparation of methyl 5-((tert-butyldimethylsilyloxy)methyl)-2-(4-chlorophenyl)-5-methylcyclohex-1-enecarboxylate (1.4): To the solution of triflate 1.3 (6.6 g, 14.8 mmol) in toluene (28 mL) and EtOH (14.8 mL) was added 2N Na₂CO₃ solution (14.8 mL). The above mixture was purged with argon for 15 min and 4-chlorophenylboronic acid (3 g, 19.24 mmol) and Pd(PPh₃)₄ (170 mg, 0.148 mmol) was added. The mixture was heated to 90° C. and the reaction was completed in 7 h. Ethanol was removed under reduced pressure and the reaction was diluted with EtOAc (150 mL). The above mixture was washed with water and brine. The organic portion was dried over anhydrous Na₂SO₄, filtered, and solvent was removed under reduced pressure. The crude material was purified by flash chromatography (Hexanes/EtOAc=10:1) to afford the title compound (5.1 g, 12.58 mmol, 85% yield). ¹H NMR (600 MHz, CDCl₃) δ 7.28 (d, J=8.4 Hz, 2H), 7.06 (d, J=8.4 Hz, 2H), 3.45 (s, 3H), 3.40 (d, J=9.5 Hz, 1H), 3.34 (d, J=9.5 Hz, 1H), 2.38-2.30 (m, 3H), 2.14-2.09 (m, 1H), 1.67 (dt, J=13.9, 7.2 Hz, 1H), 1.43 (dtd, J=12.9, 5.6, 1.4 Hz, 1H), 0.95 (s, 3H), 0.90 (s, 9H), 0.04 (d, J=2.7 Hz, 6H) ppm.

Preparation of (5-((tert-butyldimethylsilyloxy)methyl)-2-(4-chlorophenyl)-5-methylcyclohex-1-enyl)methanol (1.5): To a solution of ester 1.4 (5.1 g, 12.58 mmol) in toluene (48 mL) was added DIBAL-H (1M in toluene, 28 mL) at −78° C. and the mixture stirred for 5 h at room temp. The reaction was diluted with 50 mL toluene and was quenched by adding saturated solution of Rochelle's salt at 0° C. dropwise. The reaction was then filtered through celite and the filtrate was dried over anhydrous Na₂SO₄, filtered, and concentrated under reduced pressure. The crude material was purified by flash chromatography (Hexanes/EtOAc=3:1) to afford the title compound (4.3 g, 11.32 mmol, 90% yield). ¹H NMR (600 MHz, CDCl₃) δ 7.29 (d, J=8.4 Hz, 2H), 7.08 (d, J=8.4 Hz, 2H), 3.92 (d, J=3.9 Hz, 2H), 3.42-3.32 (m, 2H), 2.31-2.23 (m, 2H), 2.21-2.15 (m, 1H), 1.95-1.89 (m, 1H), 1.63 (ddd, J=13.0, 8.0, 6.6 Hz, 1H), 1.42 (ddt, J=12.9, 5.8, 1.3 Hz, 1H), 0.95 (s, 3H), 0.91 (s, 9H), 0.05 (s, 6H) ppm.

Preparation of ethyl 4-(4-((4-(((tert-butyldimethylsilyl)oxy)methyl)-4′-chloro-4-methyl-3,4,5,6-tetrahydro-[1,1′-biphenyl]-2-yl)methyl)piperazin-1-yl)benzoate (1.6): To a stirring solution of alcohol 1.5 (4.3 g, 11.32 mmol) in DCM (55 mL) at 0° C. was added triethylamine (3.1 ml, 22.64 mmol) followed by the addition of methanesulfonyl chloride (1.3 mL, 17 mmol). The reaction was stirred for 2 h at room temperature and then quenched with saturated NaHCO₃. The resulting mixture was diluted with 50 mL DCM and the organic portion was washed with water followed by brine. The organic portion was dried over anhydrous Na₂SO₄, filtered, and solvent was removed under reduced pressure to afford the crude product which was used in the next step without further purification.

The crude mesylate was dissolved in DMF (25 mL) followed by the addition of K₂CO₃ (3.1 g, 22.64 mmol) and ethyl 4-(piperazin-1-yl)benzoate (3.4 g, 14.71 mmol). The mixture was stirred at 75° C. for 24 h. Upon consumption of the starting material (monitored by TLC), the mixture was allowed to warm to room temperature and diluted with 150 mL EtOAc and successively washed with water (25 mL×3) and brine. The organic portion was dried over anhydrous Na₂SO₄, filtered, and solvent was removed under reduced pressure. The crude material was purified by flash chromatography (Hexanes/EtOAc=2:1) to afford the title compound (5.0 g, 8.49 mmol, 75% yield in two steps). ¹H NMR (600 MHz, CDCl₃) δ 7.90 (d, J=9.0 Hz, 2H), 7.27 (d, J=8.4 Hz, 2H), 7.00 (d, J=8.4 Hz, 2H), 6.81 (d, J=9.0 Hz, 2H), 4.32 (q, J=7.1 Hz, 2H), 3.41-3.33 (m, 2H), 3.25 (t, J=5.1 Hz, 4H), 2.80 (s, 2H), 2.39-2.32 (m, 4H), 2.28-2.19 (m, 2H), 2.17-2.11 (m, 1H), 1.94-1.89 (m, 1H), 1.63 (ddd, J=13.2, 8.6, 6.4 Hz, 1H), 1.46-1.40 (m, 1H), 1.36 (t, J=7.1 Hz, 3H), 0.94 (s, 3H), 0.91 (s, 9H), 0.05 (d, J=0.9 Hz, 6H) ppm.

Preparation of ethyl 4-(4-((4-(azidomethyl)-4′-chloro-4-methyl-3,4,5,6-tetrahydro-[1,1′-biphenyl]-2-yl)methyl)piperazin-1-yl)benzoate (1.7): Compound 1.6 (5.0 g g, 8.49 mmol) was dissolved in 40 mL THF followed by the addition of 3N HCl (10 mL) and the mixture was stirred at room temperature for 3 h. Upon consumption of the staring material (monitored by TLC), the acid was neutralized by adding solid Na₂CO₃ until effervescence stops. THF was removed under reduced pressure and the mixture was mixed with EtOAc (200 mL). The organic portion was washed with water and brine. The organic portion was dried over anhydrous Na₂SO₄, filtered, and solvent was removed under reduced pressure. The resulting crude material was used in the next step without further purification.

To a stirring solution of above crude alcohol in DCM (35 mL) at 0° C. was added triethylamine (2 mL, 15.28 mmol) followed by the addition of methanesulfonyl chloride (0.88 mL, 11.46 mmol). The reaction was stirred for 3 h at room temperature and then quenched with saturated NaHCO₃. The reaction was diluted with 40 mL DCM and the organic portion was washed with water followed by brine. The organic portion was dried over anhydrous Na₂SO₄, filtered, and solvent was removed under reduced pressure to afford the crude product which was used in the next step without further purification.

To a solution of the above crude mesylate in DMF (14 mL) was added NaN₃ (988 mg, 15.2 mmol) and KI (cat. amount). The resulting mixture was heated at 120° C. for 12 h. Upon completion of the reaction the mixture was diluted with EtOAc (150 mL) and was washed successively with water (20 mL×3) and brine. The organic portion was dried over anhydrous Na₂SO₄, filtered, and the solvent was removed under reduced pressure. The crude material was purified by flash chromatography (Hexane:EtOAc=2:1) to afford the title compound (3.48 g, 6.87 mmol, 81% yield in three steps). ¹H NMR (600 MHz, CDCl₃) δ 7.90 (d, J=9.0 Hz, 2H), 7.28 (d, J=8.4 Hz, 2H), 6.99 (d, J=8.4 Hz, 2H), 6.81 (d, J=9.0 Hz, 2H), 4.32 (q, J=7.1 Hz, 2H), 3.29-3.25 (m, 4H), 3.22 (d, J=11.8 Hz, 2H), 2.80 (s, 2H), 2.35 (t, J=5.1 Hz, 4H), 2.31-2.23 (m, 2H), 2.17-2.12 (m, 1H), 2.05-2.01 (m, 1H), 1.62 (dt, J=13.7, 6.9 Hz, 1H), 1.54-1.48 (m, 1H), 1.36 (t, J=7.1 Hz, 3H), 1.04 (s, 3H).

Preparation of ethyl 4-(4-((4-(((tert-butoxycarbonyl)amino)methyl)-4′-chloro-4-methyl-3,4,5,6-tetrahydro-[1,1′-biphenyl]-2-yl)methyl)piperazin-1-yl)benzoate (1.8): To a stirring solution of the azide 1.7 (3.48 g, 6.87 mmol) in THF (24 mL) and water (4 mL) was added triphenylphosphine (3.6 g, 13.7 mmol) and the reaction mixture was stirred for 3 h at room temperature. To the mixture was added Boc₂O (2.2 g, 10.3 mmol) and NaHCO₃ (1.7 g, 20.6 mmol) and the reaction was stirred for 9 h. Upon completion of the reaction mixture, THF was removed under reduced pressure and the reaction was diluted with EtOAc (150 mL). The organic portion was washed with water and brine. The organic solution was dried over anhydrous Na₂SO₄, filtered, and solvent was removed under reduced pressure. The crude material was purified by flash chromatography (Hexane/EtOAc=4:1) to afford the title compound (3.1 g, 5.4 mmol, 80% yield). ¹H NMR (600 MHz, CDCl₃) δ 7.91-7.87 (m, 2H), 7.29-7.26 (m, 2H), 7.01-6.97 (m, 2H), 6.83-6.77 (m, 2H), 4.74 (t, J=6.4 Hz, 1H), 4.31 (q, J=7.1 Hz, 2H), 3.25 (t, J=5.1 Hz, 4H), 3.13 (dd, J=13.5, 7.0 Hz, 1H), 3.05 (dd, J=13.5, 6.0 Hz, 1H), 2.79 (t, J=10.3 Hz, 2H), 2.38-2.22 (m, 6H), 2.10 (d, J=17.5 Hz, 1H), 1.98 (d, J=17.3 Hz, 2H), 1.58-1.49 (m, 2H), 1.43 (s, 9H), 1.36 (t, J=7.1 Hz, 3H), 0.97 (s, 3H).

Preparation of tert-butyl ((4′-chloro-4-methyl-6-((4-(4-(((4-(((R)-4-morpholino-1-(phenylthio) butan-2-yl)amino)-3-((trifluoromethyl)sulfonyl)phenyl)sulfonyl)carbamoyl)phenyl) piperazin-1-yl)methyl)-2,3,4,5-tetrahydro-[1,1′-biphenyl]-4-yl)methyl)carbamate (1.9): To a stirring solution of compound 1.8 (291 mg, 0.5 mmol) in MeOH (5 mL) and THF (1 mL) was added a solution LiOH.H₂O (42 mg, 1 mmol) in H₂O (1 mL) and the mixture was stirred for 10 h at room temperature. Once the starting material was consumed, the pH of the reaction was adjusted to 6.0 using 1N HCl. Solvents were removed from the mixture and crude was diluted with EtOAc (150 mL). The organic portion was washed with water and brine. The organic solution was dried over anhydrous Na₂SO₄, filtered, and solvent was removed under reduced pressure. The crude powder was used in the next step without further purification.

To a stirring solution of the above crude acid in DCM (5 mL) was added successively (R)-4-((4-morpholino-1-(phenylthio)butan-2-yl)amino)-3-((trifluoromethyl)sulfonyl)benzenesulfonamide (221 mg, 0.4 mmol), EDCI.HCl (238 mg, 1.25 mmol) and DMAP (152 mg, 1.25 mmol). The mixture was stirred at room temperature for 12 h. Once the amine was consumed, DCM was removed under reduced pressure and the crued product was directly loaded on a silica gel column and was purified by flash chromatography (DCM/MeOH=95:7) to afford the title compound (368 mg, 0.34 mmol, 85% yield with respect to the amine). ¹H NMR (600 MHz, Acetone-d₆) δ 8.32 (s, 1H), 8.10 (d, J=8.1 Hz, 1H), 7.86 (d, J=8.9 Hz, 2H), 7.41 (d, J=7.7 Hz, 2H), 7.37 (d, J=8.4 Hz, 2H), 7.31 (t, J=7.7 Hz, 2H), 7.22 (t, J=7.4 Hz, 1H), 7.19 (d, J=8.4 Hz, 2H), 7.00 (dd, J=25.6, 7.8 Hz, 2H), 6.89 (d, J=8.0 Hz, 2H), 6.00 (t, J=5.9 Hz, 1H), 4.21 (s, 1H), 3.55 (ddd, J=17.5, 8.6, 5.6 Hz, 4H), 3.36 (qd, J=14.0, 6.0 Hz, 2H), 3.30-3.26 (m, 4H), 3.22 (td, J=13.3, 6.2 Hz, 2H), 3.12 (dd, J=13.5, 6.8 Hz, 1H), 3.05 (dd, J=13.5, 6.3 Hz, 1H), 2.84 (q, J=12.4 Hz, 4H), 2.45-2.35 (m, 9H), 2.32-2.21 (m, 3H), 2.19 -2.10 (m, 2H), 1.82 (td, J=13.7, 5.2 Hz, 1H), 1.61 (dt, J=13.1, 6.6 Hz, 1H), 1.50 (dt, J=13.4, 6.5 Hz, 1H), 1.41 (s, 9H). ESI⁺, m/z [M+H]⁺=1089.1.

Preparation of 4-(4-((4-(aminomethyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrogen chloride (1.10): To a stirring solution of compound 1.9 (368 mg, 0.34 mmol) in DCM (5 mL) was added 4N HCl solution (0.34 mL, 1.36 mmol) in dioxane and the mixture was stirred at room temperature for 5 h. After consumption of the starting material, the solvent was removed under reduced pressure and the remaining white power was washed with Et₂O (8 mL). The ammonium salt 1.10 was used directly without further purification. ESI⁺, m/z [M+H]⁺=989.1.

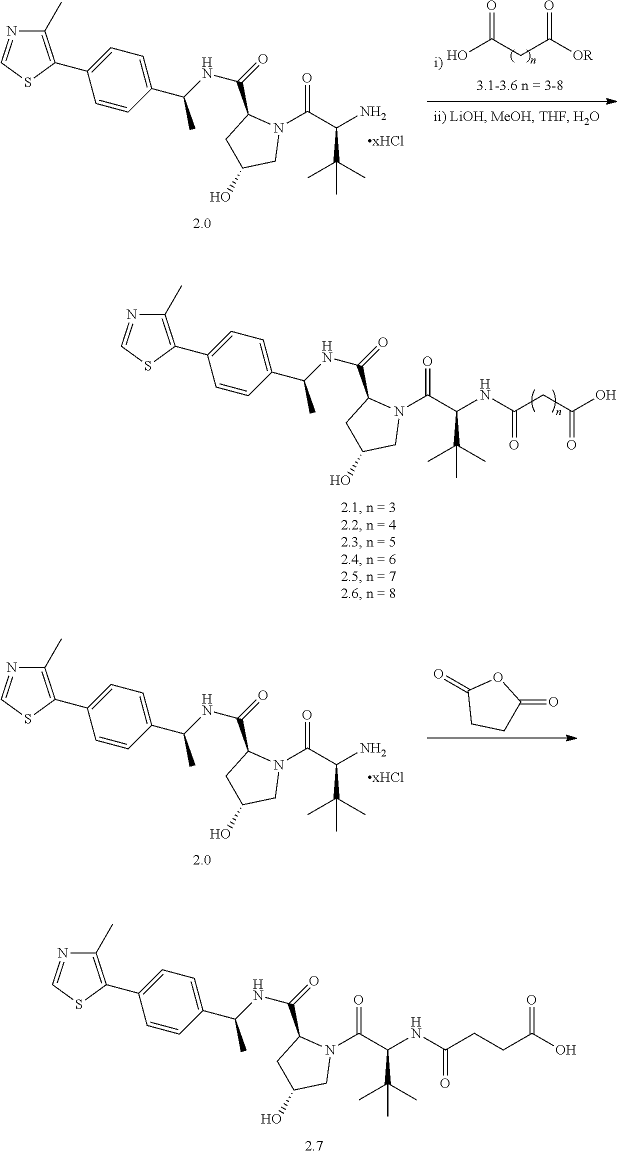

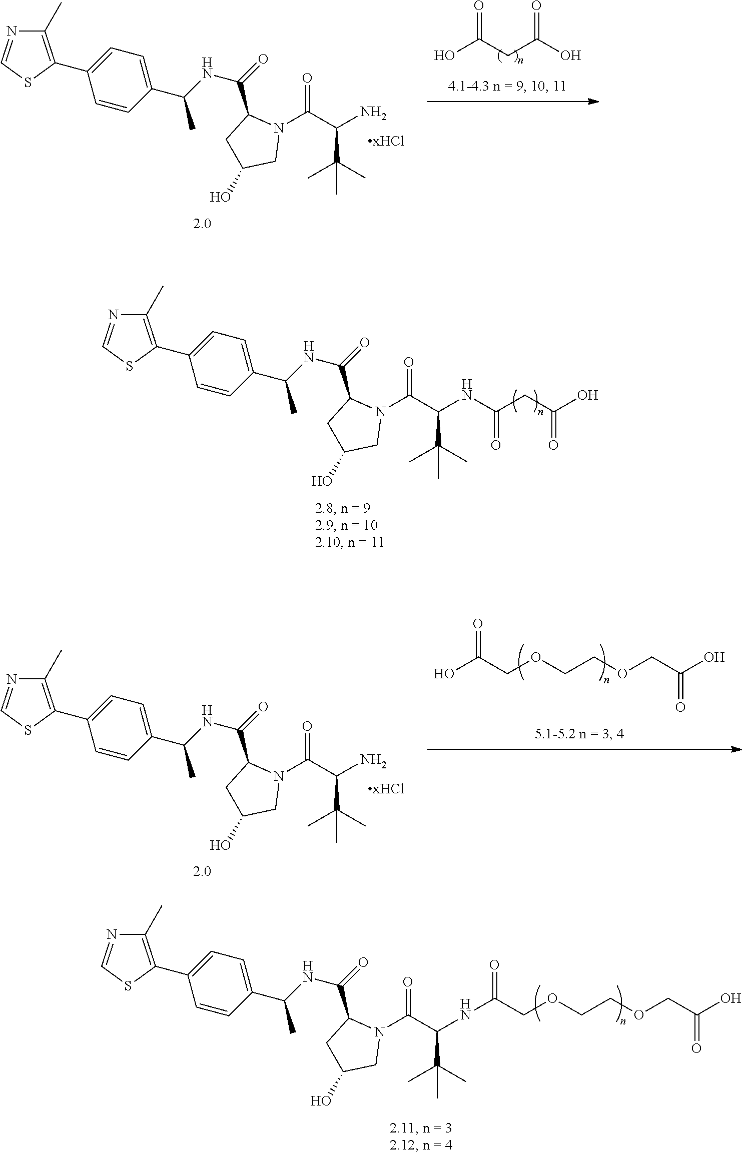

General procedure for the preparation of acids 2.1-2.6: A mixture of compound 2.0 (1.0 equiv), acid 3.x (1.1 euiv), HATU (1.2 equiv) and TEA (5 equiv) was taken in DCM and the reaction mixture was stirred at room temperature for 4 h. After the reaction was complete, the mixture was diluted with DCM and washed with saturated aqueous NH₄Cl. The organic portion was dried over anhydrous MgSO₄, filtered, and concentrated under reduced pressure. The crude ester was then diluted with THF/MeOH (1:1). To this solution LiOH.H₂O (3 equiv) aqueous solution was added and the mixture was stirred overnight. Upon completion of the reaction, pH was adjusted to 7.0 with 1N HCl. The solvents were evaporated and the residue was diluted with EtOAc. The organic portion was washed with brine and the brine was extracted with EtOAc several times. The combined organic layers were dried over anhydrous MgSO₄, filtered, and then concentrated under reduced pressure. The crude product was purified by silica gel column chromatography.

5-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic acid (2.1): ¹H NMR (400 MHz, CDCl₃) δ 8.67 (s, 1H), 7.66 (d, J=7.8 Hz, 1H), 7.45-7.32 (m, 4H), 7.19 (s, 1H), 5.15-5.02 (m, 1H), 4.80-4.69 (m, 1H), 4.57 (d, J=8.4 Hz, 1H), 4.46 (s, 1H), 4.16-4.03 (m, 1H), 3.60 (dd, J=11.1, 3.8 Hz, 1H), 2.52 (s, 3H), 2.47-1.84 (m, 8H), 1.47 (d, J=6.9 Hz, 3H), 1.05 (s, 9H) ppm.

5-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoic acid (2.2): ¹H NMR (400 MHz, CDCl₃) δ 8.67 (s, 1H), 7.66 (d, J=7.8 Hz, 1H), 7.45-7.32 (m, 4H), 7.19 (s, 1H), 5.15-5.02 (m, 1H), 4.80-4.69 (m, 1H), 4.57 (d, J=8.4 Hz, 1H), 4.46 (s, 1H), 4.16-4.03 (m, 1H), 3.60 (dd, J=11.1, 3.8 Hz, 1H), 2.52 (s, 3H), 2.47-1.84 (m, 8H), 1.47 (d, J=6.9 Hz, 3H), 1.05 (s, 9H) ppm.

7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic acid (2.3): ¹H NMR (400 MHz, CDCl₃ and CD₃OD) δ 8.72 (s, 1H), 8.05-7.89 (m, 1H), 7.43-7.33 (m, 4H), 7.24-7.08 (m, 1H), 5.14-4.95 (m, 1H), 4.73-4.40 (m, 3H), 4.00-3.93 (m, 1H), 3.76-3.59 (m, 1H), 2.52 (s, 3H), 2.38-2.05 (m, 6H), 1.71-1.49 (m, 9H), 1.04 (s, 9H), ESI⁺, m/z 587.1 [M+H]⁺.

8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoic acid (2.4): ¹H NMR (400 MHz, CDCl₃) δ 8.72 (s, 1H), 7.59 (d, J=7.8 Hz, 1H), 7.40-7.33 (m, 4H), 6.92 (d, J=8.7 Hz, 1H), 5.15-4.98 (m, 1H), 4.76-4.67 (m, 1H), 4.62 (d, J=8.9 Hz, 1H), 4.52 (s, 1H), 4.04 (d, J=11.2 Hz, 1H), 3.74-3.59 (m, 1H), 2.51 (s, 3H), 2.39-2.10 (m, 6H), 1.66-1.45 (m, 7H), 1.35-1.27 (m, 4H), 1.03 (s, 9H), ESI⁺, m/z 601.2 [M+H]⁺.

9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoic acid (2.5): ¹H NMR (600 MHz, CDCl₃) δ 8.69 (s, 1H), 7.56 (d, J=7.6 Hz, 1H), 7.42-7.35 (m, 4H), 6.78 (d, J=9.1 Hz, 1H), 5.09-5.04 (m, 1H), 4.65-4.52 (m, 2H), 4.47 (s, 1H), 3.98 (d, J=11.4 Hz, 1H), 3.63 (dd, J=11.4, 3.4 Hz, 1H), 3.40 (dt, J=3.2, 1.6 Hz, 1H), 2.52 (s, 3H), 2.29 (t, J=7.4 Hz, 2H), 2.26-2.20 (m, 3H), 2.18-2.12 (m, 1H), 1.65-1.57 (m, 4H), 1.50 (d, J=7.0 Hz, 3H), 1.36-1.29 (m, 6H), 1.03 (s, 9H), ESI⁺, m/z 615.2 [M+H]⁺.

10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecanoic acid (2.6): ¹H NMR (600 MHz, CDCl₃) δ 8.68 (s, 1H), 7.39 (dd, J=26.4, 8.2 Hz, 4H), 7.31 (d, J=7.8 Hz, 1H), 6.79 (d, J=8.6 Hz, 1H), 5.12-5.05 (m, 1H), 4.72 (t, J=8.1 Hz, 1H), 4.58 (d, J=8.9 Hz, 1H), 4.53 (s, 1H), 4.22 (d, J=11.5 Hz, 1H), 3.61 (dd, J=11.5, 3.3 Hz, 1H), 2.53 (s, 3H), 2.51-2.44 (m, 1H), 2.40-2.30 (m, 2H), 2.27-2.18 (m, 2H), 2.17 (s, 1H), 2.16-2.11 (m, 1H), 1.48 (d, J=6.9 Hz, 3H), 1.40-1.34 (m, 4H), 1.32-1.23 (m, 8H), 1.05 (s, 9H), ESI⁺, m/z 629.2 [M+H]⁺.

Preparation of 4-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-4-oxobutanoic acid (2.7): A mixture of amine salt 2, Succinic anhydride (1.1 equiv) and TEA (3 equiv) was dissolved in DCM and the reaction mixture was refluxed for 8 h. Upon completion of the reaction, DCM was evaporated and the crude solid was washed with diethyl ether to get the white powder as title compound. ¹H NMR (400 MHz, CDCl₃) δ 8.67 (s, 1H), 7.94 (d, J=8.2 Hz, 1H), 7.79 (d, J=7.7 Hz, 1H), 7.44-7.34 (m, 4H), 5.13-5.03 (m, 1H), 4.81-4.73 (m, 1H), 4.51-4.38 (m, 2H), 4.15 (d, J=11.4 Hz, 1H), 3.54 (dd, J=11.4, 3.5 Hz, 1H), 2.64-2.37 (m, 8H), 2.16-2.06 (m, 1H), 1.47 (d, J=6.9 Hz, 3H), 1.05 (s, 9H), ESI⁺, m/z 545.4 [M+H]⁺.

General procedure for the preparation of acids 2.8 to 2.12: A mixture of amine salt 2.0 (1.0 equiv), acid 4.x or 5.x (1.1 euiv), HATU (1.2 equiv), and TEA (5 equiv) was taken in DCM and the reaction mixture was stirred at room temperature for 4 h. After completion of the reaction, DCM was evaporated and the crude product was directly charged to the column purification.

11-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-11-oxoundecanoic acid (2.8): ¹H NMR (600 MHz, CDCl₃) δ 8.68 (s, 1H), 7.43-7.35 (m, 5H), 6.57 (d, J=8.9 Hz, 1H), 5.12-5.06 (m, 1H), 4.70 (t, J=8.0 Hz, 1H), 4.61 (d, J=8.9 Hz, 1H), 4.50 (s, 1H), 4.11 (d, J=11.4 Hz, 1H), 3.61 (dd, J=11.3, 3.6 Hz, 1H), 2.53 (s, 3H), 2.48-2.42 (m, 1H), 2.27 (t, J=7.3 Hz, 2H), 2.24-2.13 (m, 2H), 2.12-2.06 (m, 1H), 1.64-1.55 (m, 4H), 1.48 (d, J=6.9 Hz, 3H), 1.34-1.26 (m, 10H), 1.04 (s, 9H), ESI⁺, m/z 643.2 [M+H]⁺.

12-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-12-oxododecanoic acid (2.9): ¹H NMR (600 MHz, CDCl₃) δ 8.71 (s, 1H), 7.42 (d, J=8.2 Hz, 2H), 7.39 (d, J=8.2 Hz, 2H), 7.30-7.28 (m, 1H), 7.04 (d, J=9.1 Hz, 1H), 5.14-5.08 (m, 1H), 4.69 (dd, J=17.2, 8.7 Hz, 2H), 4.54 (s, 1H), 4.16 (d, J=11.5 Hz, 1H), 3.66 (dd, J=11.3, 3.5 Hz, 1H), 2.48 (s, 3H), 2.46 (ddd, J=12.8, 7.9, 4.5 Hz, 1H), 2.38-2.32 (m, 2H), 2.23 (dt, J=8.5, 6.3 Hz, 2H), 2.12 (dd, J=13.4, 8.0 Hz, 1H), 1.68-1.55 (m, 4H), 1.50 (d, J=6.9 Hz, 3H), 1.40-1.24 (m, 13H), 1.05 (s, 9H). ESI⁺, m/z 657 [M+H]⁺.

13-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-13-oxotridecanoic acid (2.10): ¹H NMR (600 MHz, CDCl₃) δ 8.69 (s, 1H), 7.41 (d, J=8.1 Hz, 2H), 7.37 (d, J=8.1 Hz, 2H), 7.29 (d, J=7.8 Hz, 1H), 6.74 (d, J=8.6 Hz, 1H), 5.09 (p, J=6.8 Hz, 1H), 4.71 (t, J=7.9 Hz, 1H), 4.63 (d, J=9.0 Hz, 1H), 4.52 (s, 1H), 4.18 (d, J=11.4 Hz, 1H), 3.61 (dd, J=11.3, 3.2 Hz, 1H), 2.53 (s, 3H), 2.50 (dt, J=8.0, 4.8 Hz, 1H), 2.34 (q, J=6.9 Hz, 2H), 2.21 (dt, J=15.4, 8.1 Hz, 2H), 2.14-2.07 (m, 1H), 1.62 (ddd, J=19.9, 12.9, 5.6 Hz, 5H), 1.48 (d, J=6.8 Hz, 3H), 1.36-1.21 (m, 14H), 1.04 (s, 9H).

(S)-16-((2S,4R)-4-hydroxy-2-(((S)-1-(4-(4-methylthiazol-5-yl)phenyl)ethyl)carbamoyl)pyrrolidine-1-carbonyl)-17,17-dimethyl-14-oxo-3,6,9,12-tetraoxa-15-azaoctadecanoic acid (2.11): ¹H NMR (600 MHz, CDCl₃) δ 8.65 (s, 1H), 7.78 (d, J=7.6 Hz, 1H), 7.39 (d, J=9.2 Hz, 1H), 7.36 (s, 4H), 5.10-5.03 (m, 1H), 4.72 (t, J=8.1 Hz, 1H), 4.62 (d, J=9.2 Hz, 1H), 4.49 (s, 3H), 3.99 (d, J=2.5 Hz, 2H), 3.64 (tddd, J=17.6, 14.7, 9.5, 4.5 Hz, 18H), 2.50 (s, 3H), 2.26 (ddd, J=13.1, 8.4, 4.6 Hz, 1H), 2.16 (dd, J=13.2, 8.0 Hz, 1H), 1.46 (d, J=7.0 Hz, 3H), 1.02 (s, 9H).

(S)-19-((2S,4R)-4-hydroxy-2-(((S)-1-(4-(4-methylthiazol-5-yl)phenyl)ethyl)carbamoyl)pyrrolidine-1-carbonyl)-20,20-dimethyl-17-oxo-3,6,9,12,15-pentaoxa-18-azahenicosanoic acid (2.12): ¹H NMR (600 MHz, CDCl₃) δ 8.66 (s, 1H), 7.48 (d, J=6.6 Hz, 1H), 7.37 (q, J=8.4 Hz, 5H), 5.07 (q, J=7.1 Hz, 1H), 4.74 (t, J=8.0 Hz, 1H), 4.58 (d, J=8.8 Hz, 1H), 4.51 (s, 1H), 4.12 (s, 2H), 4.08 (d, J=15.5 Hz, 2H), 3.74-3.59 (m, 18H), 2.52 (s, 3H), 2.45 (d, J=5.3 Hz, 1H), 2.13 (d, J=5.5 Hz, 1H), 1.46 (d, J=4.4 Hz, 3H), 1.04 (s, 9H).

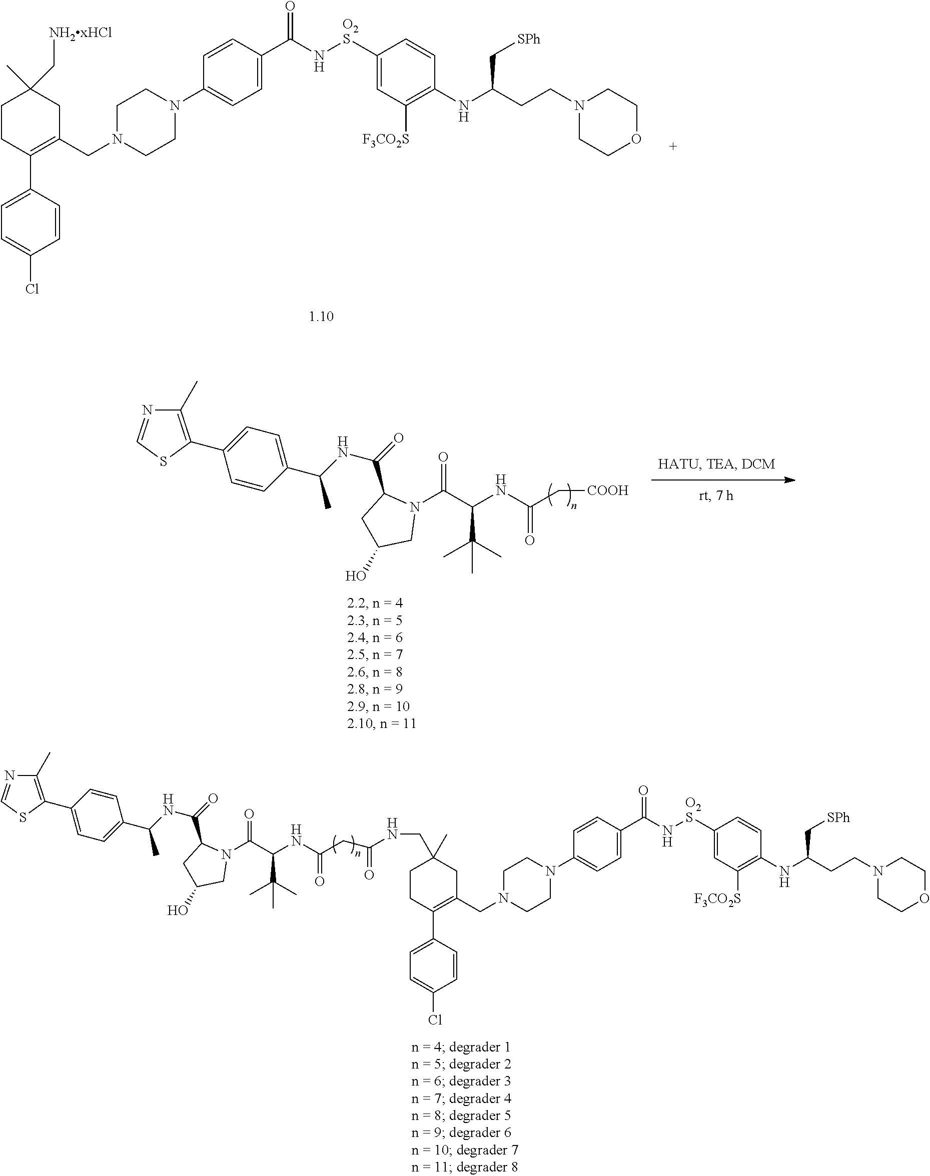

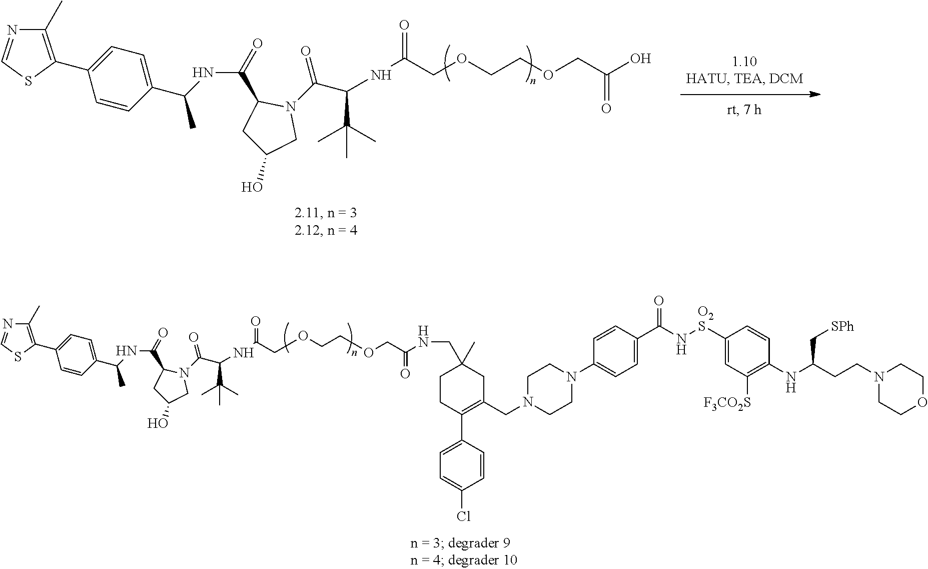

General procedure for the preparation of the DEGRADERs #1-10: To a stirring solution of amine 1.10 (12 mg, 0.011 mmol) and acid 2.x (1.1 equiv) in DCM (1 mL) was added trimethylamine (0.01 mL, 0.066 mmol) at room temperature. To the mixture HATU (5 mg, 0.012 mmol) was added and the reaction were stirred for 8 h at the same temperature. Upon completion of the reaction, the solvent was removed under reduced pressure and the crude product was purified by flash column chromatography (DCM/MeOH/TEA=96:3:1). The purified compound was mixed with 15 mL DCM and washed with saturated aqueous NH₄Cl. The organic portion was dried over Na₂SO₄, filtered, and DCM was evaporated under reduced pressure to afford the corresponding degrader.

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N6-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)adipamide (degrader #1): ¹H NMR (600 MHz, CDCl₃) δ 8.67 (s, 1H), 8.33 (d, J=2.2 Hz, 1H), 8.06 (d, J=9.4 Hz, 1H), 7.69 (dd, J=8.7, 6.3 Hz, 2H), 7.47-7.44 (m, 1H), 7.39-7.35 (m, 6H), 7.33-7.28 (m, 4H), 7.25-7.22 (m, 1H), 7.01 (dd, J=8.3, 1.8 Hz, 2H), 6.96 (d, J=8.6 Hz, 1H), 6.62 (d, J=9.2 Hz, 3H), 5.09 (t, J=7.2 Hz, 1H), 4.74 (q, J=8.4 Hz, 1H), 4.61 (dd, J=9.0, 2.9 Hz, 1H), 4.47 (s, 1H), 4.09 (t, J=9.3 Hz, 1H), 3.90 (s, 1H), 3.65 (d, J=10.1 Hz, 5H), 3.57 (d, J=11.1 Hz, 1H), 3.35 (s, 6H), 3.11 (dd, J=13.8, 5.1 Hz, 2H), 3.03-2.99 (m, 1H), 2.49 (d, J=2.1 Hz, 3H), 2.44 (d, J=5.4 Hz, 3H), 2.35 (d, J=15.4 Hz, 7H), 2.20-2.14 (m, 3H), 2.14-2.06 (m, 5H), 2.00 (d, J=7.4 Hz, 2H), 1.67 (dd, J=14.3, 7.6 Hz, 2H), 1.54 (t, J=6.9 Hz, 5H), 1.46 (d, J=7.0 Hz, 5H), 1.29-1.23 (m, 3H), 1.03 (s, 9H), 0.99 (d, J=2.9 Hz, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N7-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)heptanediamide (degrader #2): ¹H NMR (600 MHz, CDCl₃) δ 8.70 (d, J=1.3 Hz, 1H), 8.36 (t, J=2.5 Hz, 1H), 8.10 (ddd, J=9.3, 4.9, 2.3 Hz, 1H), 7.72 (dd, J=14.0, 8.6 Hz, 2H), 7.46 (d, J=2.8 Hz, 1H), 7.43-7.38 (m, 6H), 7.33 (dd, J=3.1, 1.7 Hz, 3H), 7.28 (s, 1H), 7.05-7.02 (m, 2H), 7.02-6.98 (m, 1H), 6.65 (d, J=9.6 Hz, 3H), 6.36 (s, 1H), 5.12 (t, J=7.2 Hz, 1H), 4.77 (dd, J=8.2, 5.6 Hz, 1H), 4.70 (d, J=8.9 Hz, 1H), 4.66 (d, J=8.8 Hz, 1H), 4.52 (s, 1H), 4.13 (q, J=8.8, 6.6 Hz, 1H), 3.93 (s, 1H), 3.71-3.65 (m, 4H), 3.62-3.59 (m, 1H), 3.31 (d, J=14.6 Hz, 6H), 3.13 (dd, J=13.8, 5.1 Hz, 2H), 3.05-3.01 (m, 1H), 2.53 (s, 3H), 2.45 (s, 4H), 2.41-2.32 (m, 7H), 2.23-2.16 (m, 4H), 2.13 (s, 4H), 2.08-2.00 (m, 3H), 1.73-1.66 (m, 2H), 1.57 (d, J=7.6 Hz, 4H), 1.50 (dd, J=6.9, 3.4 Hz, 5H), 1.36-1.30 (m, 2H), 1.24-1.19 (m, 2H), 1.06 (d, J=3.9 Hz, 9H), 1.01 (d, J=7.5 Hz, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N8-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)octanediamide (degrader #3): ¹H NMR (600 MHz, CDCl₃) δ 8.67 (s, 1H), 8.32-8.29 (m, 1H), 8.11-8.07 (m, 1H), 7.75 (d, J=8.6 Hz, 1H), 7.72 (d, J=8.6 Hz, 1H), 7.41-7.36 (m, 6H), 7.32-7.29 (m, 3H), 7.24 (s, 1H), 7.00 (dd, J=8.4, 1.6 Hz, 4H), 6.72 (d, J=8.5 Hz, 2H), 6.62 (d, J=9.1 Hz, 1H), 6.30 (s, 1H), 6.24 (s, 1H), 5.11-5.09 (m, 1H), 4.74 (t, J=7.5 Hz, 2H), 4.69 (d, J=8.9 Hz, 1H), 4.51 (s, 1H), 4.15 (d, J=11.5 Hz, 1H), 3.90 (s, 1H), 3.65 (s, 4H), 3.58 (d, J=11.4 Hz, 1H), 3.22 (s, 6H), 3.13-3.08 (m, 2H), 3.02 (dd, J=13.9, 7.1 Hz, 2H), 2.51 (d, J=4.8 Hz, 4H), 2.42 (s, 3H), 2.35 (s, 3H), 2.30 (s, 4H), 2.14-2.08 (m, 4H), 2.04 (s, 3H), 1.63 (s, 7H), 1.48 (d, J=7.0 Hz, 4H), 1.41 (d, J=7.2 Hz, 3H), 1.19-1.11 (m, 5H), 1.05 (d, J=3.6 Hz, 9H), 0.99 (d, J=3.1 Hz, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N9-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)nonanediamide (degrader #4): ¹H NMR (600 MHz, CDCl₃) δ 8.70 (s, 1H), 8.33 (dd, J=5.2, 2.2 Hz, 1H), 8.12-8.09 (m, 1H), 7.77 (dd, J=13.2, 8.6 Hz, 2H), 7.48-7.45 (m, 1H), 7.43-7.38 (m, 6H), 7.34-7.31 (m, 4H), 7.04-7.01 (m, 3H), 6.71 (s, 2H), 6.64 (dd, J=9.7, 3.6 Hz, 1H), 6.35 (d, J=9.5 Hz, 1H), 5.12 (td, J=7.2, 4.0 Hz, 1H), 4.78-4.73 (m, 2H), 4.53 (s, 1H), 4.15 (d, J=10.3 Hz, 1H), 3.93 (s, 2H), 3.67 (d, J=7.9 Hz, 5H), 3.62 (d, J=11.4 Hz, 1H), 3.25 (s, 6H), 3.13 (dd, J=13.8, 5.0 Hz, 2H), 3.04 (dd, J=13.9, 7.2 Hz, 2H), 2.54 (s, 3H), 2.46 (d, J=8.7 Hz, 4H), 2.35 (s, 6H), 2.19-2.11 (m, 5H), 1.70 (d, J=6.8 Hz, 5H), 1.57 (d, J=8.9 Hz, 6H), 1.53-1.48 (m, 3H), 1.45-1.39 (m, 3H), 1.24-1.19 (m, 4H), 1.13 (s, 5H), 1.07 (d, J=2.0 Hz, 9H), 1.01 (s, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N10-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)decanediamide (degrader #5): ¹H NMR (600 MHz, CDCl₃) δ 8.70 (s, 1H), 8.33 (d, J=1.9 Hz, 1H), 8.11-8.09 (m, 1H), 7.77-7.73 (m, 2H), 7.43-7.37 (m, 7H), 7.34-7.31 (m, 4H), 7.04-7.00 (m, 3H), 6.70 (d, J=8.5 Hz, 2H), 6.64 (d, J=9.2 Hz, 1H), 6.32 (dd, J=14.7, 8.9 Hz, 1H), 5.11 (td, J=7.3, 3.9 Hz, 1H), 4.78-4.68 (m, 3H), 4.53 (s, 1H), 4.16 (d, J=11.5 Hz, 1H), 3.92 (s, 1H), 3.68 (q, J=5.8, 5.3 Hz, 4H), 3.64-3.60 (m, 1H), 3.32 (s, 1H), 3.25 (s, 5H), 3.13 (dd, J=13.8, 5.0 Hz, 3H), 3.04 (dd, J=13.8, 7.2 Hz, 2H), 2.54 (s, 4H), 2.45 (s, 3H), 2.35 (s, 6H), 2.22-2.17 (m, 3H), 2.17-2.10 (m, 5H), 1.69 (dd, J=14.5, 8.0 Hz, 3H), 1.58 (dt, J=22.9, 8.2 Hz, 5H), 1.50 (t, J=6.5 Hz, 4H), 1.45 (d, J=8.0 Hz, 1H), 1.23 (s, 4H), 1.15 (dd, J=14.4, 6.8 Hz, 6H), 1.08 (s, 9H), 1.01 (d, J=4.1 Hz, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N11-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)undecanediamide (degrader #6): ¹H NMR (600 MHz, CDCl₃) δ 8.67 (s, 1H), 8.30 (d, J=3.4 Hz, 1H), 8.10 (dd, J=9.4, 2.3 Hz, 1H), 7.74 (dd, J=15.9, 8.6 Hz, 3H), 7.41-7.35 (m, 6H), 7.30 (ddt, J=10.8, 6.3, 4.5 Hz, 6H), 7.17 (d, J=7.9 Hz, 1H), 7.00 (dd, J=8.2, 1.4 Hz, 4H), 6.71 (t, J=8.0 Hz, 2H), 6.62 (d, J=9.3 Hz, 1H), 6.38 (s, 1H), 6.31 (d, J=8.9 Hz, 1H), 5.08 (dt, J=10.8, 7.1 Hz, 2H), 4.73-4.68 (m, 3H), 4.52 (s, 1H), 4.15-4.10 (m, 2H), 3.90 (s, 2H), 3.66 (m, 5H), 3.60 (dd, J=11.5, 3.5 Hz, 2H), 3.23 (m, 7H), 3.12-3.08 (m, 2H), 3.02 (dd, J=13.9, 7.2 Hz, 2H), 2.51 (d, J=1.8 Hz, 4H), 2.42 (s, 4H), 2.30 (s, 9H), 2.22-2.16 (m, 4H), 2.13-2.07 (m, 3H), 1.48-1.39 (m, 8H), 1.16 (m, 4H), 1.05 (d, J=1.6 Hz, 9H), 0.99 (s, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N12-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)dodecanediamide (degrader #7): ¹H NMR (600 MHz, CDCl₃) δ 8.67 (s, 1H), 8.31 (t, J=2.7 Hz, 1H), 8.10-8.08 (m, 1H), 7.74 (d, J=8.5 Hz, 2H), 7.40-7.33 (m, 6H), 7.32-7.26 (m, 6H), 7.04-6.98 (m, 3H), 6.69 (d, J=8.6 Hz, 2H), 6.62 (d, J=9.3 Hz, 1H), 6.33 (s, 1H), 5.11-5.05 (m, 2H), 4.70 (ddd, J=12.5, 8.4, 4.4 Hz, 3H), 4.51 (s, 1H), 4.17-4.09 (m, 2H), 3.91 (s, 1H), 3.66 (s, 4H), 3.60 (dd, J=11.5, 3.5 Hz, 2H), 3.32-3.17 (m, 7H), 3.12-3.07 (m, 2H), 3.02 (dd, J=13.9, 7.2 Hz, 2H), 2.51 (d, J=1.2 Hz, 4H), 2.43 (s, 3H), 2.34 (d, J=22.9 Hz, 6H), 2.18 (d, J=9.6 Hz, 5H), 2.09 (t, J=10.1 Hz, 4H), 1.61 (d, J=7.2 Hz, 5H), 1.51 (t, J=9.0 Hz, 5H), 1.46 (dd, J=7.0, 1.5 Hz, 3H), 1.41 (t, J=7.3 Hz, 2H), 1.18-1.11 (m, 7H), 1.05 (s, 9H), 0.99 (s, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N13-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)tridecanediamide (degrader #8): ¹H NMR (600 MHz, CDCl₃) δ 8.67 (s, 1H), 8.32 (d, J=2.0 Hz, 1H), 8.08 (dt, J=9.3, 2.1 Hz, 1H), 7.72 (d, J=8.5 Hz, 2H), 7.41-7.33 (m, 7H), 7.32-7.27 (m, 5H), 7.00 (d, J=7.7 Hz, 3H), 6.67 (d, J=8.5 Hz, 2H), 6.62 (d, J=9.3 Hz, 1H), 6.29 (d, J=8.8 Hz, 1H), 5.07 (t, J=7.2 Hz, 1H), 4.72-4.69 (m, 1H), 4.65 (dd, J=8.9, 1.7 Hz, 1H), 4.51 (d, J=4.0 Hz, 1H), 4.13 (d, J=11.4 Hz, 1H), 3.90 (s, 1H), 3.66 (s, 4H), 3.62-3.58 (m, 1H), 3.27 (s, 4H), 3.18 (s, 2H), 3.10 (dd, J=13.9, 5.1 Hz, 2H), 3.02 (dd, J=13.8, 7.2 Hz, 2H), 2.51 (m, 6H), 2.38 (m, 8H), 2.18 (m, 8H), 2.09-2.02 (m, 3H), 1.68 (s, 2H), 1.60 (d, J=6.2 Hz, 3H), 1.53 (d, J=7.7 Hz, 4H), 1.46 (dd, J=6.9, 1.9 Hz, 3H), 1.23 (d, J=7.2 Hz, 2H), 1.15 (d, J=7.2 Hz, 12H), 1.05 (s, 9H), 1.01-0.96 (m, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N14-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-3,6,9,12-tetraoxatetradecanediamide (degrader #9): ¹H NMR (600 MHz, CDCl₃) δ 8.68 (s, 1H), 8.34 (d, J=2.3 Hz, 1H), 8.11 (dd, J=9.4, 2.3 Hz, 1H), 7.68-7.66 (m, 2H), 7.52 (d, J=7.5 Hz, 1H), 7.41-7.35 (m, 7H), 7.32-7.27 (m, 5H), 7.05 (d, J=8.6 Hz, 1H), 7.00 (d, J=7.7 Hz, 3H), 6.76 (d, J=8.6 Hz, 2H), 6.62 (d, J=9.4 Hz, 1H), 5.10 (dd, J=14.2, 7.0 Hz, 2H), 4.74 (t, J=8.0 Hz, 1H), 4.63 (d, J=8.1 Hz, 1H), 4.52 (s, 1H), 4.14 (d, J=11.4 Hz, 1H), 4.04 (s, 3H), 3.93 (d, J=6.4 Hz, 3H), 3.70-3.60 (m, 16H), 3.25 (m, 5H), 3.10 (q, J=7.2 Hz, 4H), 3.02 (dd, J=13.8, 7.2 Hz, 2H), 2.49 (d, J=1.1 Hz, 3H), 2.35 (m, 4H), 2.11 (m, 4H), 2.01 (d, J=17.0 Hz, 3H), 1.71-1.66 (m, 3H), 1.59-1.54 (m, 3H), 1.47 (s, 2H), 1.39 (d, J=7.4 Hz, 4H), 1.06 (s, 9H), 0.99 (d, J=2.5 Hz, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N17-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-3,6,9,12,15-pentaoxaheptadecanediamide (degrader #10): ¹H NMR (600 MHz, CDCl₃) δ 8.70 (s, 1H), 8.35 (s, 1H), 8.14 (dd, J=9.2, 2.3 Hz, 2H), 7.70 (dd, J=9.0, 3.9 Hz, 2H), 7.43-7.37 (m, 7H), 7.33 (d, J=7.7 Hz, 3H), 7.31-7.29 (m, 2H), 7.08 (d, J=8.6 Hz, 2H), 7.02 (d, J=8.1 Hz, 2H), 6.79 (d, J=8.7 Hz, 2H), 6.64 (d, J=9.3 Hz, 2H), 5.13 (d, J=17.0 Hz, 2H), 4.77 (s, 2H), 4.65 (dd, J=8.6, 2.9 Hz, 1H), 4.54 (s, 1H), 4.16 (d, J=11.4 Hz, 1H), 4.06 (s, 2H), 4.00 (d, J=6.6 Hz, 2H), 3.95 (dd, J=15.5, 8.4 Hz, 3H), 3.73-3.59 (m, 20H), 3.37 (s, 2H), 3.26 (s, 4H), 3.04 (dd, J=13.9, 7.2 Hz, 3H), 2.82 (s, 3H), 2.54-2.51 (m, 3H), 2.44 (s, 3H), 2.37-2.32 (m, 4H), 2.12 (m, 4H), 2.04 (t, J=13.6 Hz, 3H), 1.50 (d, J=6.9 Hz, 4H), 1.09 (s, 9H), 1.02 (s, 3H).

### Example 25: Preparation of Degraders #11-13

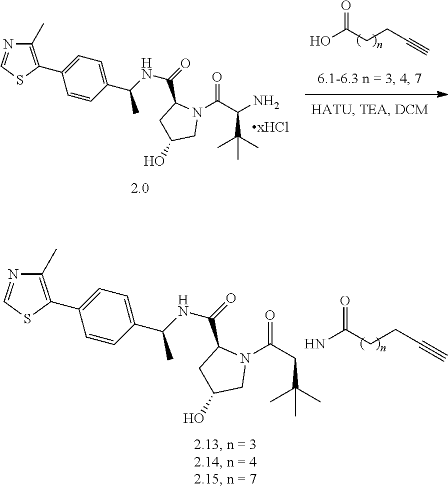

General procedure for the preparation of compounds 2.13 to 2.15: A mixture of amine 2.0 (1.0 equiv.), acid 6 (1.1 equiv.), HATU (1.2 equiv.) and TEA (5 equiv.) was taken in DCM and the reaction mixture was stirred at room temperature for 4 h. After the completion of the reaction, solvent was evaporated, and the crude mixture was purified by column chromatography to afford the desired compound.

(2S,4R)-1-((S)-2-(hept-6-ynamido)-3,3-dimethylbutanoyl)-4-hydroxy-N—((S)-1-(4-(4-methylthiazol-5-yl)phenyl)ethyl)pyrrolidine-2-carboxamide (2.13): 1H NMR (600 MHz, CDCl₃) δ 8.67 (s, 1H), 7.41-7.40 (m, 3H), 7.37 (d, J=8.2 Hz, 2H), 6.09 (d, J=8.2 Hz, 1H), 5.08 (p, J=7.0 Hz, 1H), 4.75 (t, J=7.9 Hz, 1H), 4.53 (d, J=8.5 Hz, 2H), 4.15 (d, J=11.5 Hz, 1H), 3.71 (tdd, J=10.9, 5.7, 3.3 Hz, 0.5H), 3.59 (dd, J=11.4, 3.5 Hz, 1H), 3.17 (ttd, J=7.5, 4.4, 2.2 Hz, 0.5H), 2.59 (dt, J=12.7, 5.6 Hz, 1H), 2.53 (s, 3H), 2.26 (t, J=7.4 Hz, 2H), 2.21 (td, J=7.0, 2.6 Hz, 2H), 2.09-2.02 (m, 1H), 1.95 (t, J=2.6 Hz, 1H), 1.75 (p, J=7.5 Hz, 2H), 1.48 (dd, J=11.1, 6.9 Hz, 5H), 1.05 (s, 9H).

(2S,4R)-1-((S)-3,3-dimethyl-2-(oct-7-ynamido)butanoyl)-4-hydroxy-N—((S)-1-(4-(4-methylthiazol-5-yl)phenyl)ethyl)pyrrolidine-2-carboxamide (2.14): 1H NMR (600 MHz, CDCl₃) δ 8.67 (s, 1H), 7.43-7.40 (m, 3H), 7.36 (d, J=8.2 Hz, 2H), 6.08 (d, J=8.5 Hz, 1H), 5.07 (q, J=7.1 Hz, 1H), 4.74 (t, J=7.9 Hz, 1H), 4.53 (d, J=8.6 Hz, 2H), 4.15 (d, J=11.5 Hz, 1H), 3.59 (dd, J=11.4, 3.6 Hz, 1H), 3.23 (q, J=7.3 Hz, 1H), 2.73 (s, 1H), 2.59 (ddd, J=12.6, 7.3, 4.8 Hz, 1H), 2.53 (s, 3H), 2.23 (t, J=7.6 Hz, 2H), 2.19 (td, J=7.0, 2.7 Hz, 2H), 2.10-2.04 (m, 1H), 1.93 (t, J=2.6 Hz, 1H), 1.56-1.50 (m, 3H), 1.47 (d, J=6.9 Hz, 3H), 1.05 (s, 9H).

(2S,4R)-1-((S)-3,3-dimethyl-2-(undec-10-ynamido)butanoyl)-4-hydroxy-N—((S)-1-(4-(4-methylthiazol-5-yl)phenyl)ethyl)pyrrolidine-2-carboxamide (2.15): 1H NMR (600 MHz, CDCl₃) δ 8.67 (s, 1H), 7.43 (d, J=7.7 Hz, 1H), 7.41 (d, J=7.8 Hz, 2H), 7.36 (d, J=8.2 Hz, 2H), 6.13-6.05 (m, 1H), 5.08 (p, J=7.0 Hz, 1H), 4.73 (q, J=7.8, 7.2 Hz, 1H), 4.56-4.49 (m, 2H), 4.18-4.09 (m, 1H), 3.59 (d, J=11.3 Hz, 1H), 3.23 (q, J=7.3 Hz, 1H), 2.58 (dq, J=12.8, 6.8, 6.1 Hz, 1H), 2.53 (s, 3H), 2.23-2.16 (m, 4H), 2.09-2.02 (m, 1H), 1.93 (t, J=2.6 Hz, 1H), 1.52-1.49 (m, 2H), 1.47 (d, J=6.9 Hz, 3H), 1.41-1.39 (m, 2H), 1.29 (s, 6H), 1.05 (s, 9H).

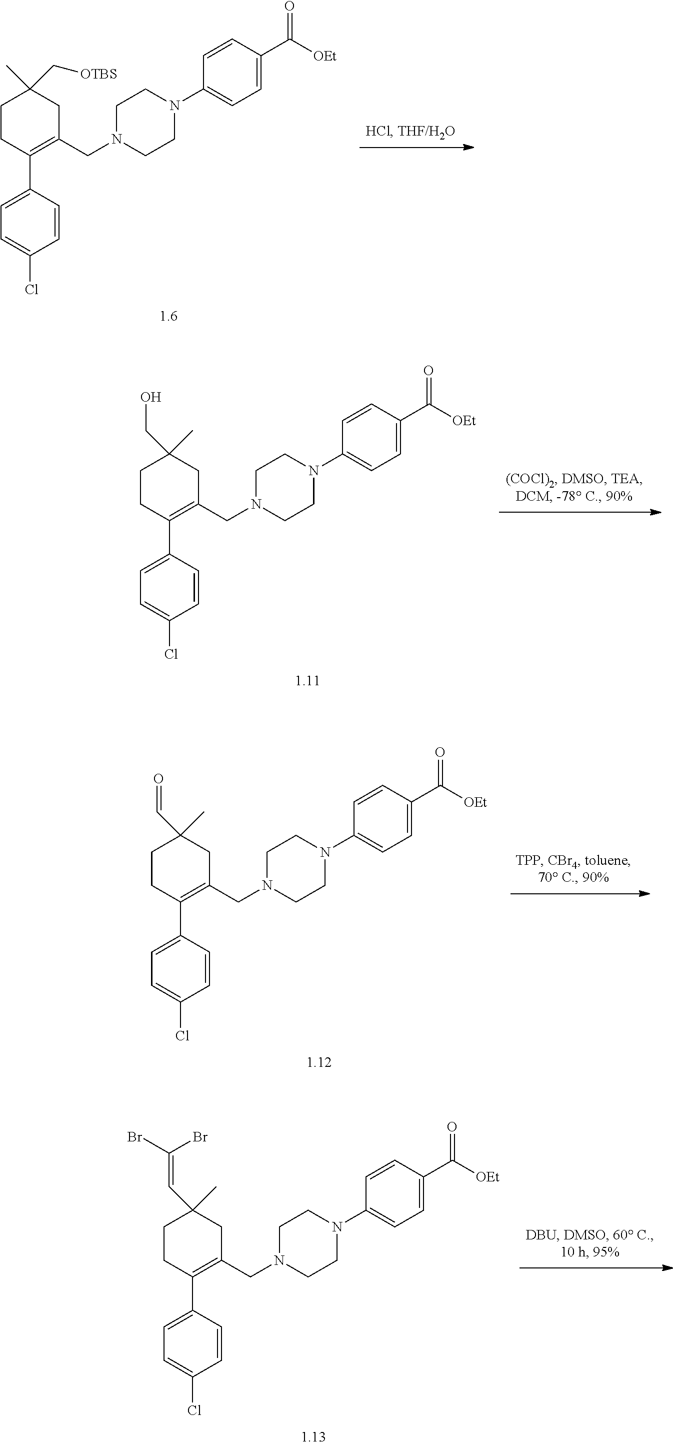 [SMILES: CCOC(=O)c1ccc(N2CCN(CC3=C(c4ccc(Cl)cc4)CCC(C)(CO[Si](C)(C)C(C)(C)C)C3)CC2)cc1 ; CCOC(=O)c1ccc(N2CCN(CC3=C(c4ccc(Cl)cc4)CCC(C)(CO)C3)CC2)cc1 ; CCOC(=O)c1ccc(N2CCN(CC3=C(c4ccc(Cl)cc4)CCC(C)(C=O)C3)CC2)cc1 ; CCOC(=O)c1ccc(N2CCN(CC3=C(c4ccc(Cl)cc4)CCC(C)(C=C(Br)Br)C3)CC2)cc1]

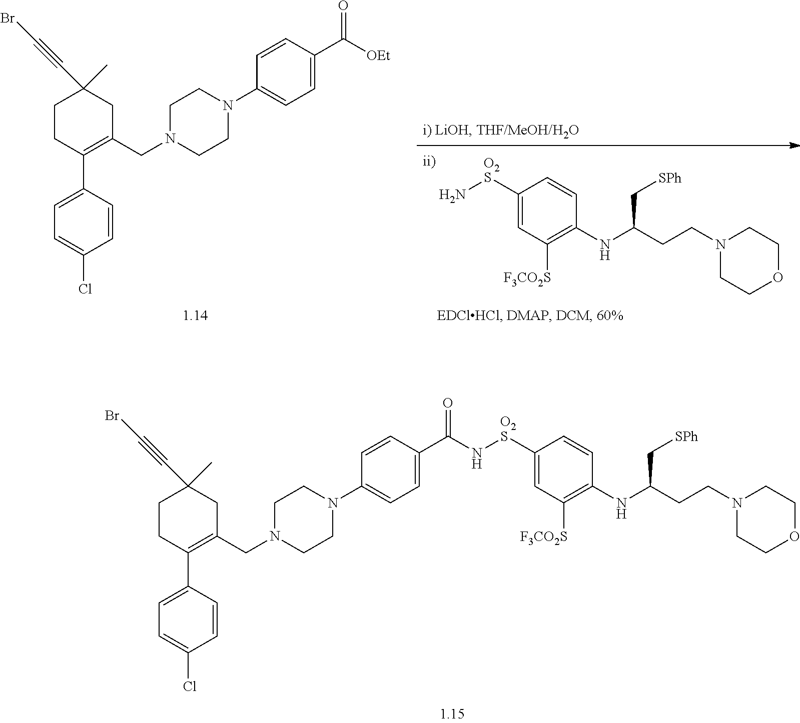

Preparation of ethyl 4-(4-((4′-chloro-4-(hydroxymethyl)-4-methyl-3,4,5,6-tetrahydro-[1,1′-biphenyl]-2-yl)methyl)piperazin-1-yl)benzoate (1.11): Alcohol 1.11 was synthesized as described in the synthesis of compound 1.7. 1H NMR (600 MHz, CDCl₃) δ 7.89 (d, J=9.0 Hz, 2H), 7.28 (d, J=8.4 Hz, 2H), 7.00 (d, J=8.4 Hz, 2H), 6.80 (d, J=9.0 Hz, 2H), 4.31 (q, J=7.1 Hz, 2H), 3.46 (d, J=2.9 Hz, 2H), 3.25 (t, J=5.1 Hz, 4H), 2.85-2.76 (m, 2H), 2.36 (tt, J=11.4, 6.1 Hz, 4H), 2.29 (d, J=7.0 Hz, 2H), 2.16 (dt, J=17.5, 2.3 Hz, 1H), 2.00 (d, J=17.3 Hz, 1H), 1.62 (dd, J=13.4, 6.9 Hz, 1H), 1.52-1.45 (m, 1H), 1.36 (t, J=7.1 Hz, 3H), 1.01 (s, 3H).

Preparation of ethyl 4-(4-((4′-chloro-4-formyl-4-methyl-3,4,5,6-tetrahydro-[1,1′-biphenyl]-2-yl)methyl)piperazin-1-yl)benzoate (1.12): To a stirring solution of oxalyl chloride (1.5 equiv.) in DCM was added DMSO (3 equiv.) dropwise at −78° C. and the mixture was stirred for 30 min at the same temperature. Alcohol 1.11 dissolved in DCM/DMSO was added to the above mixture dropwise and the mixture was stirred for 45 min. TEA (6 equiv.) was added to the above mixture and the temperature was allowed to warm to room temperature. Once the reaction was complete, the mixture was diluted with DCM and washed successively with saturate aqueous NaHCO₃, water, and brine. The organic portion was dried over anhydrous MgSO₄, filtered, and concentrated under reduced pressure. The crude product was purified by column chromatography. ¹H NMR (600 MHz, CDCl₃) δ 9.52 (s, 1H), 8.36 (s, 1H), 8.11 (dd, J=9.2, 1.9 Hz, 1H), 7.63 (d, J=8.6 Hz, 2H), 7.37 (d, J=7.5 Hz, 2H), 7.31 (t, J=7.4 Hz, 2H), 7.27 (d, J=7.5 Hz, 2H), 7.08 (d, J=8.3 Hz, 1H), 6.94 (d, J=8.4 Hz, 2H), 6.79 (d, J=8.7 Hz, 2H), 6.61 (d, J=9.3 Hz, 1H), 3.91 (s, 1H), 3.65 (t, J=7.6 Hz, 4H), 3.29 (t, J=4.7 Hz, 4H), 3.10 (dd, J=13.9, 5.0 Hz, 1H), 3.02 (dd, J=13.8, 7.2 Hz, 1H), 2.85 (s, 2H), 2.66 (d, J=17.6 Hz, 1H), 2.45-2.37 (m, 6H), 2.36-2.26 (m, 7H), 2.12 (dd, J=12.9, 4.7 Hz, 1H), 2.00 (dd, J=13.6, 6.8 Hz, 1H), 1.65 (ddt, J=36.1, 13.7, 6.5 Hz, 3H), 1.14 (s, 3H).

Preparation of ethyl 4-(4-((4′-chloro-4-(2,2-dibromovinyl)-4-methyl-3,4,5,6-tetrahydro-[1,1′-biphenyl]-2-yl)methyl)piperazin-1-yl)benzoate (1.13): To a solution of aldehyde 1.12 (1 equiv.) was mixed with triphenyl phosphate (8 equiv.) and CBr₄ (5 equiv.). The reaction mixture was heated to 70° C. and stirred for 10 h. Upon completion of the reaction (monitored by TLC), the solvent was evaporated in reduced pressure and the crude product was purified by column chromatography to afford the title compound. ¹H NMR (600 MHz, CDCl₃) δ 7.90 (d, J=9.0 Hz, 2H), 7.29 (d, J=8.4 Hz, 2H), 6.99 (d, J=8.4 Hz, 2H), 6.81 (d, J=9.0 Hz, 2H), 6.59 (s, 1H), 4.32 (q, J=7.1 Hz, 2H), 3.27 (t, J=6.1 Hz, 4H), 2.85-2.71 (m, 2H), 2.58 (s, 1H), 2.34 (dp, J=17.1, 5.6, 5.1 Hz, 5H), 2.28-2.19 (m, 2H), 2.11 (d, J=17.1 Hz, 1H), 1.57-1.51 (m, 1H), 1.36 (t, J=7.1 Hz, 3H), 1.32 (s, 3H).

Preparation of ethyl 4-(4-((4-(bromoethynyl)-4′-chloro-4-methyl-3,4,5,6-tetrahydro-[1,1′-biphenyl]-2-yl)methyl)piperazin-1-yl)benzoate (1.14): To a stirring solution of compound 1.13 (1 equiv.) in DMSO was added DBU (2 equiv.) and the mixture was stirred for 6 h at 65° C. Upon completion of the reaction, the mixture was diluted with EtOAc and washed with water several times followed by washing with brine. The organic portion was dried over anhydrous MgSO₄, filtered, and evaporated under reduced pressure. The crude product was purified by silica gel column chromatography to afford the title compound. ¹H NMR (600 MHz, CDCl₃) δ 7.89 (d, J=9.0 Hz, 2H), 7.29 (d, J=8.4 Hz, 2H), 7.00 (d, J=8.4 Hz, 2H), 6.81 (d, J=9.1 Hz, 2H), 4.32 (q, J=7.1 Hz, 2H), 3.26 (t, J=5.2 Hz, 4H), 2.82-2.77 (m, 2H), 2.62 (d, J=16.6 Hz, 1H), 2.60-2.53 (m, 1H), 2.43 (dt, J=10.6, 5.2 Hz, 2H), 2.30 (dt, J=10.8, 5.1 Hz, 2H), 2.22 (d, J=16.9 Hz, 1H), 2.07 (d, J=16.8 Hz, 1H), 1.89-1.84 (m, 1H), 1.59-1.53 (m, 1H), 1.36 (t, J=7.1 Hz, 3H), 1.33 (s, 3H).

Preparation of 4-(4-((4-(bromoethynyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (1.15): Compound 1.15 was prepared from compound 1.14 in the same way as compound 1.9 was prepared from the compound 1.8. ¹H NMR (600 MHz, CDCl₃) δ 8.36 (d, J=2.3 Hz, 1H), 8.11 (dd, J=9.4, 2.3 Hz, 1H), 7.62 (d, J=8.6 Hz, 2H), 7.40-7.35 (m, 2H), 7.32-7.27 (m, 4H), 7.10-7.06 (m, 1H), 6.99 (d, J=8.4 Hz, 2H), 6.79 (d, J=8.8 Hz, 2H), 6.61 (d, J=9.4 Hz, 1H), 3.90 (d, J=3.6 Hz, 1H), 3.65 (d, J=3.2 Hz, 4H), 3.29 (t, J=5.2 Hz, 4H), 3.10 (dd, J=13.8, 5.1 Hz, 1H), 3.02 (dd, J=13.9, 7.3 Hz, 1H), 2.80 (s, 2H), 2.62 (d, J=17.2 Hz, 1H), 2.56 (d, J=9.0 Hz, 1H), 2.46-2.41 (m, 4H), 2.39-2.34 (m, 2H), 2.34-2.28 (m, 4H), 2.21 (d, J=18.1 Hz, 1H), 2.15-2.09 (m, 1H), 1.86 (dd, J=10.0, 2.9 Hz, 1H), 1.67 (dd, J=14.6, 8.8 Hz, 2H), 1.59-1.52 (m, 2H), 1.32 (s, 3H).

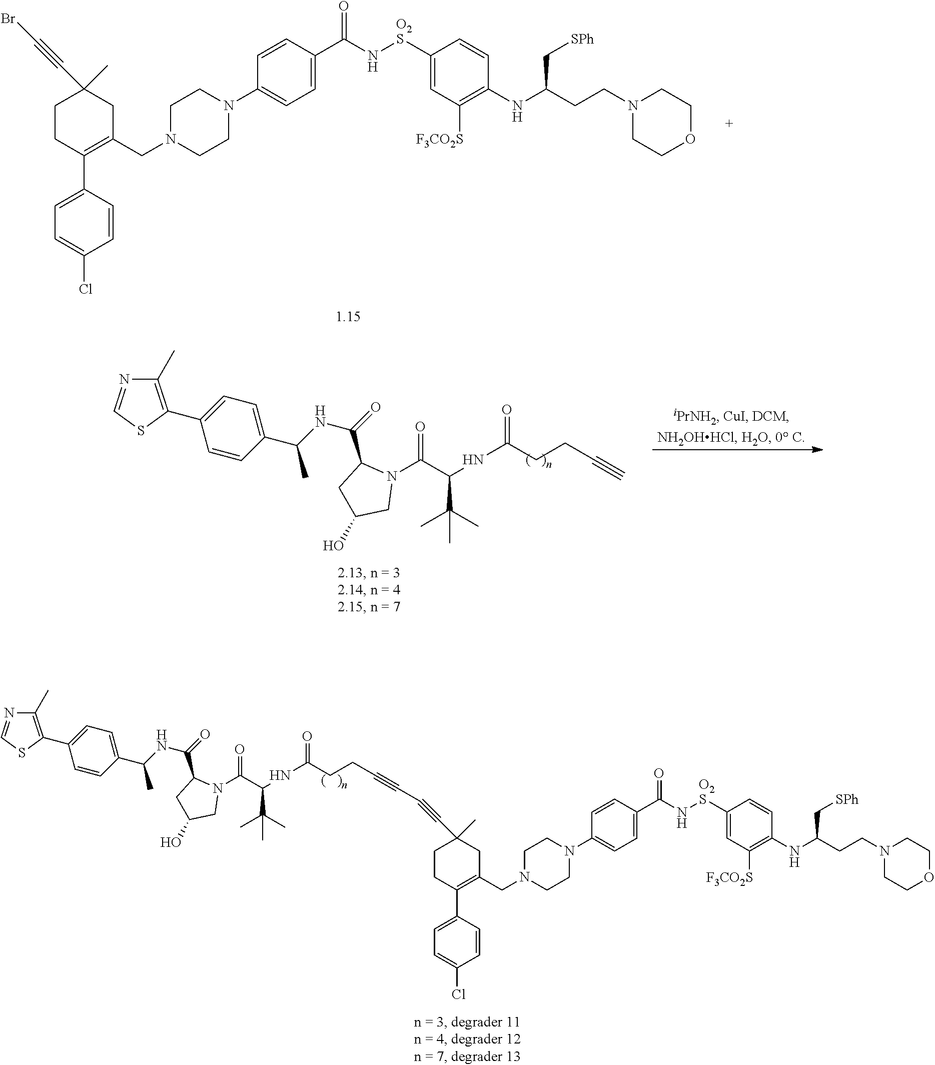

General procedure for the preparation of the degraders #11-13. Compound 1.15 (1.2 equiv.), terminal alkynes (1 equiv.), CuI (5 mol %) were taken in a reaction vessel and the vessel was purged with argon three times. To the above mixture, DCM was added along with iPrNH₂ (5 equiv.) and started stirring at 0° C. An aqueous solution of NH₂OH.HCl was added to the above reaction mixture whenever the reaction color turns blue until the blue color disappears. Once the reaction was complete (monitored by TLC), the reaction was diluted with DCM and washed with brine. The organic portion was dried over anhydrous MgSO₄, filtered, and then concentrated under reduced pressure. The crude product was purified by silica gel column chromatography to afford the title compounds.

(2S,4R)-1-((2S)-2-(9-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)nona-6,8-diynamido)-3,3-dimethylbutanoyl)-4-hydroxy-N—((S)-1-(4-(4-methylthiazol-5-yl)phenyl)ethyl)pyrrolidine-2-carboxamide (degrader #11): ¹H NMR (600 MHz, CDCl₃) δ 8.67 (s, 1H), 8.34 (t, J=2.8 Hz, 1H), 8.11 (t, J=6.8 Hz, 1H), 7.77 (t, J=9.3 Hz, 2H), 7.40 (dd, J=8.2, 3.0 Hz, 3H), 7.36 (d, J=8.3 Hz, 4H), 7.31-7.28 (m, 4H), 7.05 (d, J=8.5 Hz, 1H), 7.02 (dd, J=8.4, 2.0 Hz, 2H), 6.79-6.72 (m, 2H), 6.61 (dd, J=11.6, 9.7 Hz, 1H), 6.32 (t, J=8.4 Hz, 1H), 5.08 (td, J=14.8, 13.6, 8.1 Hz, 1H), 4.80-4.67 (m, 2H), 4.57-4.47 (m, 1H), 4.16 (q, J=13.5 Hz, 1H), 3.93-3.85 (m, 1H), 3.65 (s, 4H), 3.56 (dd, J=11.6, 3.0 Hz, 1H), 3.23 (s, 4H), 3.10 (dd, J=13.8, 4.8 Hz, 1H), 3.01 (dd, J=13.5, 6.8 Hz, 1H), 2.83-2.73 (m, 3H), 2.70-2.61 (m, 1H), 2.59-2.54 (m, 2H), 2.52 (d, J=4.3 Hz, 3H), 2.46-2.39 (m, 2H), 2.39-2.26 (m, 6H), 2.20 (t, J=8.2 Hz, 5H), 2.09 (dd, J=24.1, 10.2 Hz, 3H), 2.02 (s, 1H), 1.89-1.82 (m, 1H), 1.74 (p, J=7.3 Hz, 2H), 1.67 (d, J=9.1 Hz, 1H), 1.61-1.53 (m, 3H), 1.47 (t, J=7.1 Hz, 3H), 1.33 (s, 3H), 1.04 (d, J=6.0 Hz, 7H), 1.00 (s, 4H).

(2S,4R)-1-((2S)-2-(10-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)deca-7,9-diynamido)-3,3-dimethylbutanoyl)-4-hydroxy-N—((S)-1-(4-(4-methylthiazol-5-yl)phenyl)ethyl)pyrrolidine-2-carboxamide (degrader #12): ¹H NMR (600 MHz, CDCl₃) δ 8.67 (s, 1H), 8.30 (d, J=8.4 Hz, 1H), 8.07 (s, 1H), 7.87 (t, J=10.8 Hz, 2H), 7.39-7.34 (m, 7H), 7.31-7.27 (m, 4H), 7.02 (d, J=8.3 Hz, 2H), 6.99 (d, J=10.0 Hz, 1H), 6.72 (s, 2H), 6.58 (s, 1H), 5.13-5.03 (m, 1H), 4.74-4.65 (m, 1H), 4.48 (s, 1H), 4.18 (d, J=9.9 Hz, 1H), 3.87 (s, 1H), 3.64 (s, 4H), 3.53 (s, 1H), 3.22 (s, 4H), 3.08 (dd, J=13.7, 4.4 Hz, 1H), 3.00 (dd, J=13.7, 7.2 Hz, 1H), 2.87-2.62 (m, 5H), 2.57 (s, 2H), 2.52 (d, J=2.5 Hz, 3H), 2.46-2.22 (m, 11H), 2.18 (d, J=12.4 Hz, 5H), 2.12-2.05 (m, 2H), 1.97 (d, J=15.8 Hz, 1H), 1.85 (dd, J=12.9, 5.9 Hz, 1H), 1.66 (dt, J=13.1, 7.0 Hz, 2H), 1.61 (s, 1H), 1.53 (td, J=12.3, 6.0 Hz, 3H), 1.49-1.42 (m, 4H), 1.37 (s, 1H), 1.32 (s, 3H), 1.01 (s, 9H).

(2S,4R)-1-((2S)-2-(13-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)trideca-10,12-diynamido)-3,3-dimethylbutanoyl)-4-hydroxy-N—((S)-1-(4-(4-methylthiazol-5-yl)phenyl)ethyl)pyrrolidine-2-carboxamide (degrader #13): ¹H NMR (600 MHz, CDCl₃) δ 8.67 (s, 1H), 8.30 (s, 1H), 8.09 (d, J=10.7 Hz, 1H), 7.74 (t, J=8.4 Hz, 2H), 7.41-7.33 (m, 6H), 7.32-7.26 (m, 5H), 7.04 (dd, J=8.4, 3.1 Hz, 1H), 7.01 (dd, J=8.3, 3.1 Hz, 2H), 6.74 (d, J=7.6 Hz, 2H), 6.61 (dd, J=9.2, 5.0 Hz, 1H), 6.31 (dd, J=31.5, 8.5 Hz, 1H), 5.08 (q, J=7.7 Hz, 1H), 4.70 (dt, J=28.7, 8.4 Hz, 2H), 4.51 (s, 1H), 4.15 (dd, J=10.4, 4.6 Hz, 1H), 3.90 (s, 1H), 3.70-3.62 (m, 4H), 3.60 (dt, J=11.3, 3.8 Hz, 1H), 3.27 (s, 4H), 3.09 (dd, J=13.7, 4.9 Hz, 1H), 3.05-2.98 (m, 1H), 2.82-2.59 (m, 5H), 2.52 (s, 3H), 2.51-2.29 (m, 9H), 2.27 (t, J=6.8 Hz, 2H), 2.25-2.13 (m, 4H), 2.08 (d, J=7.6 Hz, 5H), 2.00 (d, J=16.7 Hz, 1H), 1.85 (dd, J=12.8, 6.0 Hz, 1H), 1.71-1.64 (m, 1H), 1.57-1.49 (m, 3H), 1.49-1.41 (m, 6H), 1.40-1.34 (m, 2H), 1.32 (d, J=2.3 Hz, 2H), 1.22-1.17 (m, 4H), 1.04 (d, J=3.2 Hz, 9H).

### Example 26: Preparation of Degraders #14-20

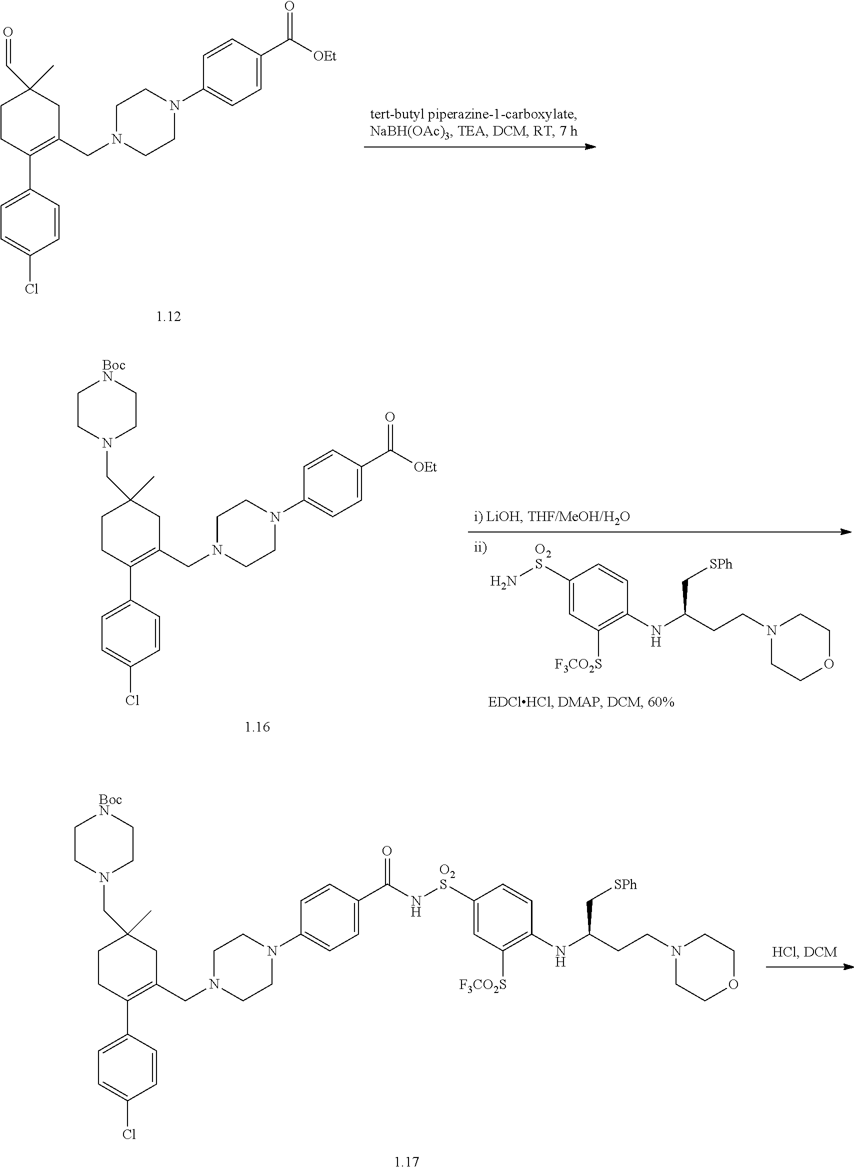

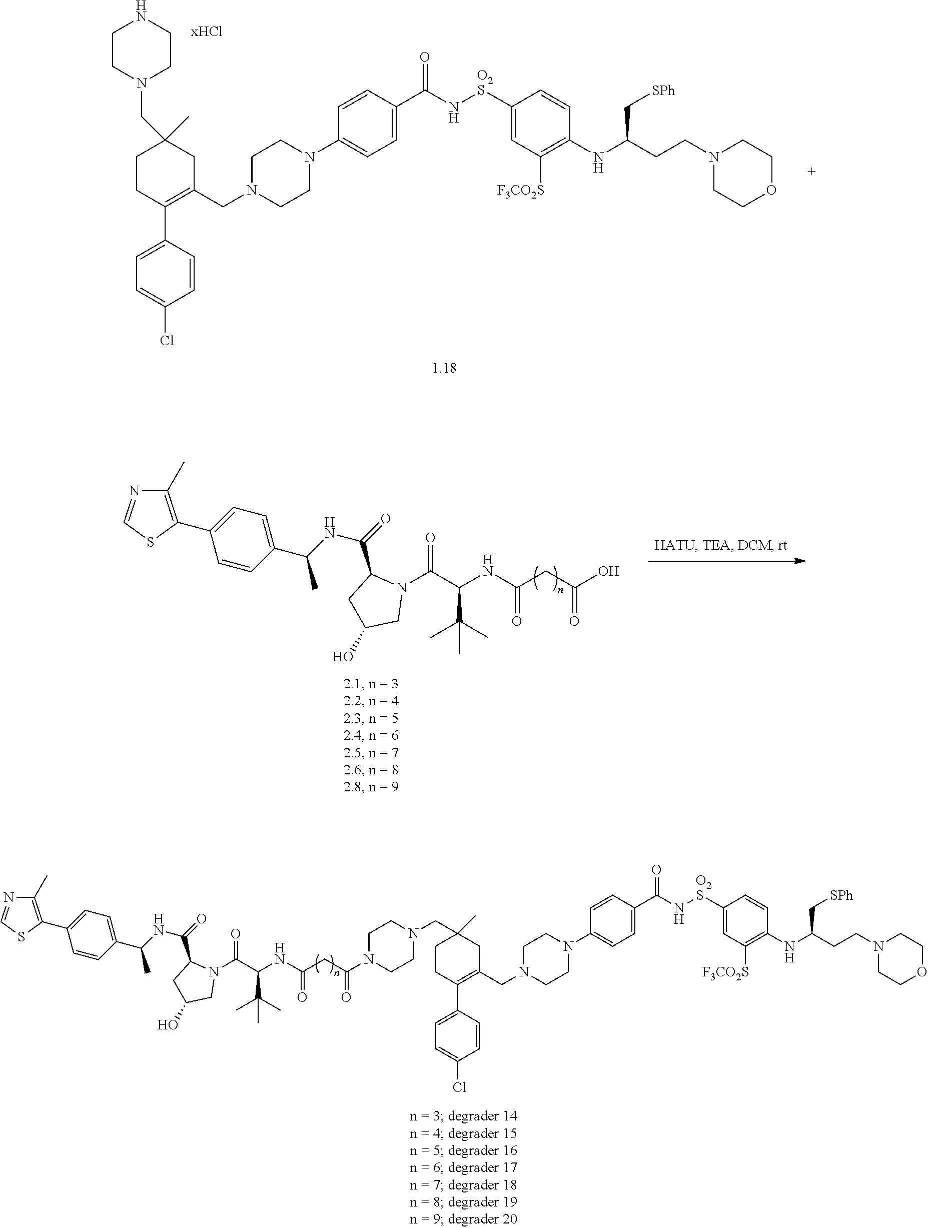

Preparation of tert-butyl 4-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)piperazine-1-carboxylate (1.16): To a stirring solution of aldehyde 1.12 (1 equiv.) in DCM was added tert-butyl piperazine-1-carboxylate (1.5 equiv.), NaBH(OAc)₃ (7 equiv.) and TEA (10 equiv.). The resulting mixture was stirred at room temperature for 8 h. After the completion of the reaction, the reaction mixture was diluted with DCM and then washed with water followed by brine. The organic portion was dried over anhydrous MgSO₄, filtered, and concentrated under reduced pressure. The crude product was purified by silica gel column chromatography to afford the title compound. ¹H NMR (600 MHz, CDCl₃) δ 7.89 (d, J=9.0 Hz, 2H), 7.27 (d, J=8.4 Hz, 2H), 6.99 (d, J=8.4 Hz, 2H), 6.81 (d, J=9.0 Hz, 2H), 4.32 (q, J=7.1 Hz, 2H), 3.40 (s, 4H), 3.24 (t, J=5.0 Hz, 4H), 2.79 (s, 2H), 2.55-2.44 (m, 4H), 2.35 (qt, J=11.0, 4.8 Hz, 4H), 2.31-2.25 (m, 1H), 2.25-2.17 (m, 3H), 2.13 (d, J=17.4 Hz, 1H), 1.93 (d, J=17.3 Hz, 1H), 1.62-1.59 (m, 2H), 1.45 (s, 9H), 1.36 (t, J=7.1 Hz, 3H), 0.95 (s, 3H).

Preparation of tert-butyl 4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carboxylate (1.17): Compound 1.17 was prepared from compound 1.16 following the same way as compound 1.9 was prepared from the compound 1.8. ¹H NMR (600 MHz, CDCl₃) δ 8.36 (d, J=2.3 Hz, 1H), 8.11 (dd, J=9.2, 2.3 Hz, 1H), 7.63 (d, J=8.8 Hz, 2H), 7.37 (d, J=7.2 Hz, 2H), 7.31 (t, J=7.4 Hz, 2H), 7.28 (d, J=8.4 Hz, 2H), 7.07 (d, J=8.6 Hz, 1H), 6.98 (d, J=8.4 Hz, 2H), 6.78 (d, J=9.2 Hz, 2H), 6.61 (d, J=9.4 Hz, 1H), 3.94-3.86 (m, 1H), 3.66 (p, J=7.2, 6.1 Hz, 4H), 3.40 (s, 4H), 3.27 (s, 4H), 3.10 (dd, J=13.9, 5.1 Hz, 1H), 3.02 (dd, J=13.9, 7.3 Hz, 1H), 2.83 (s, 2H), 2.49 (s, 4H), 2.44 (s, 2H), 2.39-2.34 (m, 5H), 2.34-2.25 (m, 4H), 2.25-2.18 (s, 3H), 2.14-2.11 (m, 2H), 1.92 (d, J=17.2 Hz, 1H), 1.67 (td, J=14.1, 5.6 Hz, 1H), 1.60 (dt, J=14.3, 7.6 Hz, 1H), 1.45 (s, 9H), 1.46-1.44 (m, 1H), 0.94 (s, 3H). ESI+, m/z [M+H]⁺=1158.3.

Preparation of 4-(4-((4′-chloro-4-methyl-4-(piperazin-1-ylmethyl)-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (1.18): Compound 1.18 was prepared from compound 1.17 following the same procedure as compound 1.10 was prepared from compound 1.9. ESI⁺, m/z [M+H]⁺=1058.5.

General procedure of the preparation of degraders #14-20: Degraders #14-20 was prepared following the same procedure as degrader 1 was prepared with amine 1.18 in place of amine 1.10.

(2S,4R)-1-((2S)-2-(5-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide (degrader #14): ¹H NMR (600 MHz, CDCl₃) δ 8.67 (s, 1H), 8.32 (s, 1H), 8.09 (d, J=9.1 Hz, 1H), 7.70 (t, J=9.2 Hz, 2H), 7.57-7.51 (m, 1H), 7.37 (q, J=7.8, 7.3 Hz, 6H), 7.31-7.23 (m, 5H), 7.02 (d, J=8.5 Hz, 1H), 6.98 (d, J=8.3 Hz, 2H), 6.91 (d, J=7.1 Hz, 1H), 6.74 (dd, J=8.5, 4.0 Hz, 2H), 6.60 (d, J=9.4 Hz, 1H), 5.13-5.06 (m, 1H), 4.73 (t, J=7.7 Hz, 1H), 4.54 (dd, J=8.2, 5.5 Hz, 1H), 4.47 (s, 1H), 4.11 (d, J=7.5 Hz, 1H), 3.93-3.85 (m, 1H), 3.69-3.63 (m, 4H), 3.57 (dt, J=21.2, 10.6 Hz, 3H), 3.40 (s, 2H), 3.26 (s, 4H), 3.10 (dd, J=13.8, 5.0 Hz, 1H), 3.02 (dd, J=13.8, 7.1 Hz, 1H), 2.92 (s, 2H), 2.53 (d, J=17.2 Hz, 3H), 2.49 (d, J=2.2 Hz, 3H), 2.44 (s, 6H), 2.40-2.16 (m, 15H), 2.15-2.03 (m, 2H), 1.98-1.83 (m, 3H), 1.72-1.56 (m, 2H), 1.46 (d, J=6.8 Hz, 3H), 1.42 (d, J=6.4 Hz, 1H), 1.28 (s, 1H), 1.05 (s, 9H), 0.93 (d, J=2.7 Hz, 3H).

(2S,4R)-1-((2S)-2-(6-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (degrader #15): ¹H NMR (600 MHz, CDCl₃) δ 8.67 (s, 1H), 8.33-8.30 (m, 1H), 8.10 (d, J=8.9 Hz, 1H), 7.70 (t, J=8.6 Hz, 2H), 7.47 (dd, J=17.6, 7.8 Hz, 1H), 7.40-7.35 (m, 6H), 7.27 (td, J=19.9, 17.7, 7.3 Hz, 5H), 7.03 (d, J=8.1 Hz, 1H), 6.98 (d, J=8.0 Hz, 2H), 6.74 (dd, J=8.8, 4.2 Hz, 2H), 6.60 (d, J=9.4 Hz, 1H), 6.56 (t, J=9.5 Hz, 1H), 5.13-5.04 (m, 1H), 4.75 (q, J=7.8 Hz, 1H), 4.63 (dd, J=8.7, 4.5 Hz, 1H), 4.48 (s, 1H), 4.11 (d, J=11.0 Hz, 1H), 3.90 (d, J=6.6 Hz, 1H), 3.67 (t, J=8.4 Hz, 4H), 3.61-3.49 (m, 3H), 3.39 (s, 2H), 3.25 (s, 4H), 3.10 (dd, J=13.9, 4.9 Hz, 1H), 3.02 (dd, J=13.8, 7.1 Hz, 1H), 2.89 (s, 2H), 2.50 (s, 9H), 2.47-2.29 (m, 11H), 2.29-2.04 (m, 12H), 1.91 (d, J=16.8 Hz, 1H), 1.68 (dd, J=14.1, 8.1 Hz, 1H), 1.59 (s, 3H), 1.46 (d, J=6.9 Hz, 3H), 1.42 (dd, J=11.9, 5.7 Hz, 1H), 1.28 (s, 1H), 1.05 (s, 9H), 0.94 (s, 3H).

(2S,4R)-1-((2S)-2-(7-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (degrader #16): ¹H NMR (600 MHz, CDCl₃) δ 8.67 (s, 1H), 8.32 (s, 1H), 8.10 (d, J=9.1 Hz, 1H), 7.69 (dd, J=12.3, 9.0 Hz, 2H), 7.45-7.34 (m, 7H), 7.29 (dd, J=17.6, 7.9 Hz, 5H), 7.04 (d, J=8.2 Hz, 1H), 6.98 (d, J=8.3 Hz, 2H), 6.74 (dd, J=8.7, 4.8 Hz, 2H), 6.61 (d, J=9.4 Hz, 1H), 6.35 (dd, J=19.3, 8.7 Hz, 1H), 5.09 (dt, J=13.6, 6.8 Hz, 1H), 4.73 (dt, J=10.8, 8.0 Hz, 1H), 4.63 (t, J=8.6 Hz, 1H), 4.48 (s, 1H), 4.10 (d, J=11.2 Hz, 1H), 3.94-3.86 (m, 1H), 3.66 (s, 4H), 3.58 (d, J=10.3 Hz, 3H), 3.40 (s, 2H), 3.25 (s, 4H), 3.10 (dd, J=13.8, 4.9 Hz, 1H), 3.02 (dd, J=13.8, 7.2 Hz, 1H), 2.93-2.80 (m, 2H), 2.63-2.41 (m, 11H), 2.41-2.30 (m, 6H), 2.30-2.03 (m, 11H), 1.91 (d, J=20.2 Hz, 1H), 1.59 (ddd, J=30.7, 14.5, 7.2 Hz, 7H), 1.46 (dd, J=6.8, 2.3 Hz, 3H), 1.45-1.38 (m, 1H), 1.35-1.27 (m, 3H), 1.04 (s, 9H), 0.96-0.92 (m, 3H).

(2S,4R)-1-((2S)-2-(8-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #17): ¹H NMR (600 MHz, CDCl₃) δ 8.67 (s, 1H), 8.32 (s, 1H), 8.10 (d, J=9.1 Hz, 1H), 7.71 (t, J=7.5 Hz, 2H), 7.44 (dd, J=27.6, 7.8 Hz, 1H), 7.37 (dd, J=15.4, 6.6 Hz, 6H), 7.31-7.23 (m, 5H), 7.03 (d, J=8.4 Hz, 1H), 6.98 (d, J=8.2 Hz, 2H), 6.74 (d, J=7.4 Hz, 2H), 6.60 (d, J=9.4 Hz, 1H), 6.33 (dd, J=13.9, 8.9 Hz, 1H), 5.12-5.05 (m, 1H), 4.72 (q, J=7.9 Hz, 1H), 4.64-4.59 (m, 1H), 4.49 (s, 1H), 4.10 (d, J=11.3 Hz, 1H), 3.89 (s, 1H), 3.66 (s, 4H), 3.58 (d, J=11.4 Hz, 3H), 3.41 (s, 2H), 3.24 (s, 4H), 3.09 (dd, J=13.8, 4.8 Hz, 1H), 3.02 (dd, J=13.8, 7.1 Hz, 1H), 2.87 (s, 2H), 2.50 (s, 8H), 2.38 (ddt, J=24.5, 18.9, 9.7 Hz, 10H), 2.23 (dq, J=25.6, 7.6 Hz, 8H), 2.14-2.05 (m, 2H), 1.91 (dd, J=16.5, 6.8 Hz, 1H), 1.66 (s, 1H), 1.58-1.56 (m, 2H), 1.45 (d, J=6.6 Hz, 4H), 1.26 (d, J=14.6 Hz, 8H), 1.04 (s, 9H), 0.94 (s, 3H).

(2S,4R)-1-((2S)-2-(9-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide (degrader #18): ¹H NMR (600 MHz, CDCl₃) δ 8.67 (s, 1H), 8.32 (d, J=2.3 Hz, 1H), 8.11 (dd, J=9.3, 2.3 Hz, 1H), 7.69 (dd, J=8.9, 6.3 Hz, 2H), 7.47 (dd, J=39.4, 7.9 Hz, 1H), 7.40-7.33 (m, 6H), 7.31-7.24 (m, 5H), 7.04 (d, J=8.5 Hz, 1H), 7.00-6.97 (m, 2H), 6.75 (dd, J=9.2, 2.6 Hz, 2H), 6.60 (d, J=9.4 Hz, 1H), 6.30 (dd, J=24.3, 8.9 Hz, 1H), 5.13-5.06 (m, 1H), 4.74-4.70 (m, 1H), 4.63 (dd, J=8.9, 5.2 Hz, 1H), 4.49 (s, 1H), 4.11 (d, J=11.4 Hz, 1H), 3.93-3.85 (m, 1H), 3.70-3.62 (m, 4H), 3.61-3.52 (m, 3H), 3.41 (s, 2H), 3.24 (s, 4H), 3.08 (d, J=5.0 Hz, 1H), 3.02 (dd, J=13.9, 7.2 Hz, 1H), 2.85 (s, 1H), 2.55-2.44 (m, 9H), 2.40-2.29 (m, 8H), 2.27-2.08 (m, 11H), 1.92-1.87 (m, 1H), 1.56 (d, J=15.1 Hz, 5H), 1.51-1.40 (m, 5H), 1.26 (d, J=8.2 Hz, 9H), 1.04 (s, 9H), 0.94 (s, 3H).

(2S,4R)-1-((2S)-2-(10-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-10-oxodecanamido)-3,3-dimethylbutanoyl)-4-hydroxy-N—((S)-1-(4-(4-methylthiazol-5-yl)phenyl)ethyl)pyrrolidine-2-carboxamide (degrader #19): ¹H NMR (600 MHz, CDCl₃) δ 8.67 (s, 1H), 8.32 (s, 1H), 8.10 (dd, J=9.2, 2.0 Hz, 1H), 7.70 (dd, J=8.9, 4.4 Hz, 2H), 7.44-7.33 (m, 7H), 7.32-7.23 (m, 5H), 7.03 (d, J=8.6 Hz, 1H), 6.98 (d, J=8.3 Hz, 2H), 6.74 (d, J=8.8 Hz, 2H), 6.60 (d, J=9.5 Hz, 1H), 6.29 (dd, J=17.3, 8.8 Hz, 1H), 5.07 (td, J=7.2, 3.4 Hz, 1H), 4.70 (q, J=7.8 Hz, 1H), 4.61 (dd, J=8.9, 4.3 Hz, 1H), 4.49 (s, 1H), 4.10 (d, J=11.5 Hz, 1H), 3.89 (s, 1H), 3.65 (q, J=5.9 Hz, 5H), 3.61-3.53 (m, 3H), 3.42 (s, 2H), 3.25 (s, 4H), 3.09 (dd, J=13.9, 5.0 Hz, 1H), 3.03-2.98 (m, 1H), 2.87 (s, 2H), 2.58-2.41 (m, 11H), 2.41-2.25 (m, 10H), 2.25-2.05 (m, 9H), 1.95-1.86 (m, 1H), 1.68 (ddd, J=19.8, 14.8, 6.8 Hz, 2H), 1.45 (dd, J=6.9, 2.9 Hz, 3H), 1.25 (s, 12H), 1.04 (s, 9H), 0.94 (s, 3H).

(2S,4R)-1-((2S)-2-(11-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-11-oxoundecanamido)-3,3-dimethylbutanoyl)-4-hydroxy-N—((S)-1-(4-(4-methylthiazol-5-yl)phenyl)ethyl)pyrrolidine-2-carboxamide (degrader #20): ¹H NMR (600 MHz, CDCl₃) δ 8.67 (s, 1H), 8.32 (d, J=2.1 Hz, 1H), 8.09 (dd, J=9.2, 2.0 Hz, 1H), 7.72 (dd, J=9.0, 2.9 Hz, 2H), 7.43-7.34 (m, 7H), 7.31-7.23 (m, 5H), 7.01 (d, J=8.6 Hz, 1H), 6.98 (d, J=8.4 Hz, 2H), 6.75 (d, J=9.0 Hz, 2H), 6.59 (d, J=9.4 Hz, 1H), 6.25 (t, J=9.2 Hz, 1H), 5.12-5.04 (m, 1H), 4.71 (td, J=7.9, 3.2 Hz, 1H), 4.60 (dd, J=8.8, 2.0 Hz, 1H), 4.50 (s, 1H), 4.11 (d, J=11.5 Hz, 1H), 3.89 (dt, J=8.0, 4.3 Hz, 1H), 3.65 (dt, J=14.4, 7.1 Hz, 5H), 3.61-3.56 (m, 3H), 3.43 (s, 2H), 3.25 (d, J=5.2 Hz, 4H), 3.09 (dd, J=13.9, 5.0 Hz, 1H), 3.01 (dd, J=13.9, 7.2 Hz, 1H), 2.86 (s, 2H), 2.59-2.45 (m, 9H), 2.44-2.26 (m, 12H), 2.25-2.04 (m, 9H), 1.93 (d, J=17.2 Hz, 1H), 1.67 (dt, J=14.2, 7.0 Hz, 1H), 1.46 (dd, J=6.9, 1.7 Hz, 4H), 1.25 (s, 14H), 1.04 (s, 9H), 0.94 (s, 3H).

### Example 27: Preparation of Degraders #21-23

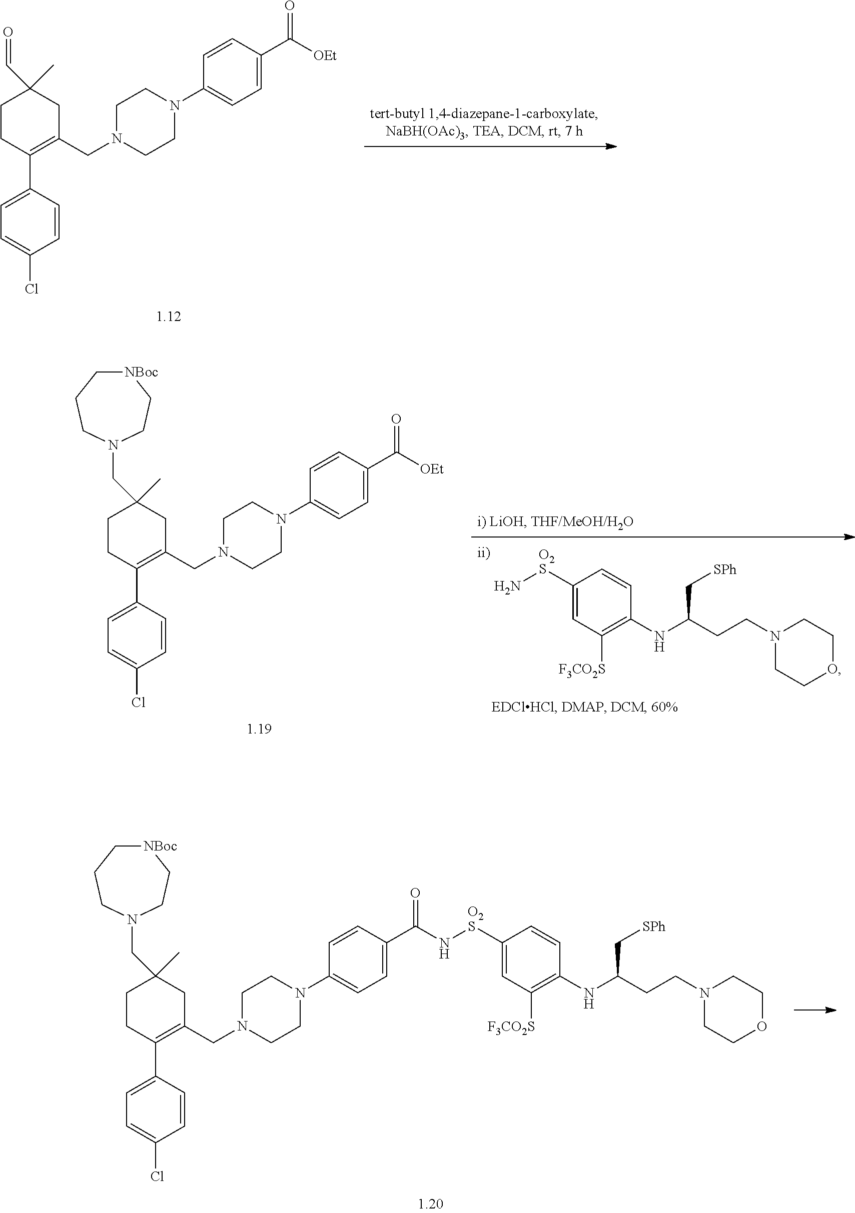

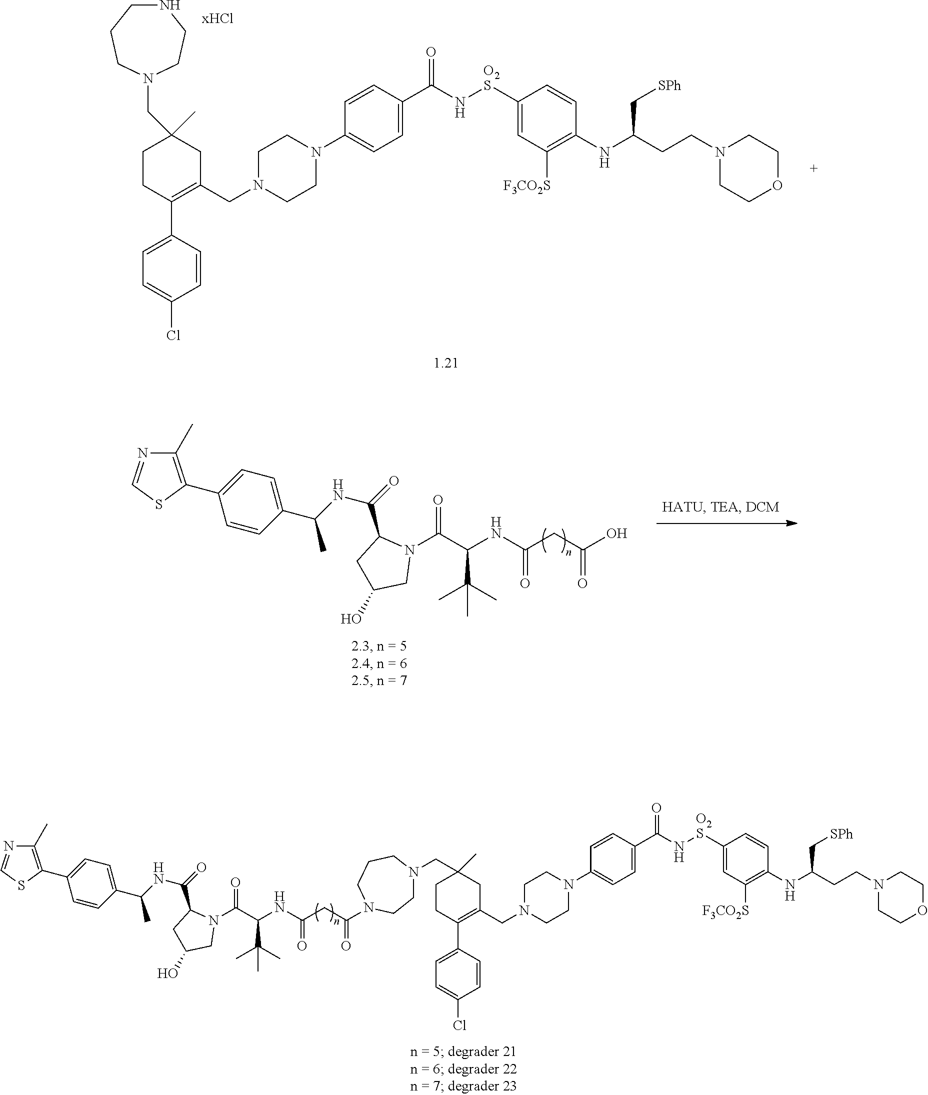

Preparation of tert-butyl 4-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)-1,4-diazepane-1-carboxylate (1.19): To a stirring solution of aldehyde 1.12 (1 equiv.) in DCM was added tert-butyl 1,4-diazepane-1-carboxylate (1.5 equiv.), NaBH(OAc)₃ (7 equiv.) and TEA (10 equiv.). The resulting mixture was stirred at room temperature for 8 h. After the completion of the reaction, the reaction mixture was diluted with DCM and then washed with water followed by brine. The organic portion was dried over anhydrous MgSO₄, filtered, and concentrated under reduced pressure. The crude product was purified by silica gel column chromatography to afford the title compound. ¹H NMR (600 MHz, CDCl₃) δ 7.89 (d, J=9.0 Hz, 2H), 7.27 (d, J=8.5 Hz, 2H), 6.99 (d, J=8.4 Hz, 2H), 6.81 (d, J=9.0 Hz, 2H), 4.31 (q, J=7.1 Hz, 2H), 3.45 (s, 2H), 3.40 (s, 2H), 3.25 (t, J=4.8 Hz, 4H), 2.82 (s, 1H), 2.79 (s, 3H), 2.77-2.73 (m, 2H), 2.45-2.31 (m, 6H), 2.26-2.17 (s, 2H), 2.09 (d, J=17.3 Hz, 1H), 1.90 (d, J=17.3 Hz, 1H), 1.78 (d, J=26.9 Hz, 2H), 1.58 (dd, J=13.8, 7.7 Hz, 2H), 1.46 (s, 9H), 1.36 (t, J=7.1 Hz, 3H), 0.93 (s, 3H). ESI⁺, m/z [M+H]⁺=665.3.

Preparation of tert-butyl 4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-1,4-diazepane-1-carboxylate (1.20): Compound 1.20 was prepared from compound 1.19 following the same procedure as compound 1.9 was prepared from compound 1.8. ¹H NMR (600 MHz, CDCl₃) δ 8.35 (s, 1H), 8.10 (d, J=9.2 Hz, 1H), 7.65 (s, 2H), 7.37 (d, J=7.6 Hz, 2H), 7.30 (d, J=6.2 Hz, 2H), 7.28 (d, J=8.0 Hz, 2H), 7.05 (s, 1H), 6.98 (d, J=8.0 Hz, 2H), 6.76 (s, 2H), 6.60 (d, J=7.9 Hz, 1H), 3.90 (s, 1H), 3.69-3.61 (m, 4H), 3.44 (s, 2H), 3.40 (s, 2H), 3.26 (s, 4H), 3.10 (dd, J=13.8, 4.7 Hz, 1H), 3.02 (dd, J=13.6, 7.1 Hz, 1H), 2.88-2.72 (m, 6H), 2.42 (s, 3H), 2.39-2.34 (m, 5H), 2.33-2.28 (m, 3H), 2.22 (s, 1H), 2.14-2.09 (m, 2H), 1.91 (d, J=20.3 Hz, 3H), 1.78 (d, J=21.3 Hz, 4H), 1.68 (t, J=14.1 Hz, 2H), 1.45 (s, 9H), 0.93 (s, 3H). ESI⁺, m/z [M+H]⁺=1172.4.

Preparation of 4-(4-((4-((1,4-diazepan-1-yl)methyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (1.21): Compound 1.21 was prepared from compound 1.20 following the same procedure as compound 1.10 was prepared from compound 1.9. ESI⁺, m/z [M+H]⁺=1072.4.

General procedure for the preparation of degraders #21-23: Degraders #21-23 were prepared following the same procedure as degrader 1 was prepared with amine 1.21 in place of amine 1.10.

(2S,4R)-1-((2S)-2-(7-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-1,4-diazepan-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (degrader #21): ¹H NMR (600 MHz, CDCl₃) δ 8.67 (s, 1H), 8.32 (d, J=1.7 Hz, 1H), 8.09 (d, J=9.2 Hz, 1H), 7.73 (dt, J=8.6, 4.4 Hz, 2H), 7.44-7.34 (m, 7H), 7.31-7.22 (m, 5H), 7.01 (d, J=8.4 Hz, 1H), 6.98 (d, J=8.1 Hz, 2H), 6.75 (d, J=8.4 Hz, 2H), 6.60 (dd, J=9.4, 2.5 Hz, 1H), 6.43 (t, J=7.5 Hz, 1H), 5.12-5.05 (m, 1H), 4.76-4.65 (m, 2H), 4.49 (s, 1H), 4.14-4.08 (m, 1H), 3.91-3.85 (m, 1H), 3.65 (tt, J=11.3, 6.0 Hz, 5H), 3.61-3.48 (m, 3H), 3.48-3.42 (m, 2H), 3.24 (s, 4H), 3.09 (dd, J=13.8, 4.9 Hz, 1H), 3.01 (dd, J=13.9, 7.2 Hz, 1H), 2.90-2.81 (m, 4H), 2.80-2.70 (m, 2H), 2.50 (d, J=1.9 Hz, 3H), 2.46-2.30 (m, 12H), 2.28-2.03 (m, 7H), 1.96-1.72 (m, 2H), 1.70-1.64 (m, 1H), 1.54 (dd, J=23.0, 7.2 Hz, 5H), 1.48-1.44 (m, 3H), 1.31 (s, 3H), 1.25 (s, 3H), 1.04 (s, 9H), 0.90 (s, 3H).

(2S,4R)-1-((2S)-2-(8-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-1,4-diazepan-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #22): ¹H NMR (600 MHz, CDCl₃) δ 8.66 (s, 1H), 8.31 (s, 1H), 8.09 (d, J=9.1 Hz, 1H), 7.74 (d, J=7.8 Hz, 2H), 7.47-7.32 (m, 7H), 7.32-7.22 (m, 5H), 7.01 (d, J=6.9 Hz, 1H), 6.99-6.96 (m, 2H), 6.75 (d, J=8.7 Hz, 2H), 6.60 (d, J=9.3 Hz, 1H), 6.41 (dt, J=26.2, 9.4 Hz, 1H), 5.11-5.02 (m, 1H), 4.73-4.65 (m, 2H), 4.49 (s, 1H), 4.11 (t, J=12.5 Hz, 1H), 3.92-3.86 (m, 1H), 3.70-3.50 (m, 8H), 3.50-3.41 (m, 2H), 3.24 (s, 4H), 3.09 (dd, J=13.9, 4.9 Hz, 1H), 3.01 (dd, J=13.8, 7.2 Hz, 1H), 2.86 (s, 4H), 2.76 (d, J=27.1 Hz, 2H), 2.50 (s, 3H), 2.47-2.34 (m, 10H), 2.34-2.18 (m, 8H), 2.10 (dt, J=18.6, 7.8 Hz, 2H), 1.84 (dd, J=74.4, 9.9 Hz, 2H), 1.68 (dt, J=14.1, 6.9 Hz, 1H), 1.54 (d, J=40.2 Hz, 5H), 1.48-1.41 (m, 3H), 1.39-1.18 (m, 8H), 1.04 (s, 9H), 0.90 (s, 3H).

(2S,4R)-1-((2S)-2-(9-(4-((4′-chloro-4-methyl-6-((4-(4-(((4-(((R)-4-morpholino-1-(phenylthio)butan-2-yl)amino)-3-(2S,4R)-1-((2S)-2-(9-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-1,4-diazepan-1-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide (degrader #23): ¹H NMR (600 MHz, CDCl₃) δ 8.67 (s, 1H), 8.32 (q, J=2.6 Hz, 1H), 8.12 (d, J=9.2 Hz, 1H), 7.75-7.69 (m, 2H), 7.47-7.32 (m, 7H), 7.32-7.24 (m, 5H), 7.04 (d, J=8.5 Hz, 1H), 6.98 (dd, J=8.4, 3.3 Hz, 2H), 6.80-6.76 (m, 2H), 6.60 (d, J=9.5 Hz, 1H), 6.43-6.29 (m, 1H), 5.07 (q, J=9.7, 8.4 Hz, 1H), 4.75-4.66 (m, 2H), 4.49 (s, 1H), 4.18-4.08 (m, 1H), 3.89 (dt, J=8.0, 4.2 Hz, 1H), 3.69-3.61 (m, 5H), 3.61-3.43 (m, 5H), 3.23 (q, J=10.4 Hz, 4H), 3.10 (dd, J=13.9, 4.9 Hz, 1H), 3.01 (dd, J=13.9, 7.2 Hz, 1H), 2.86 (dt, J=11.3, 5.3 Hz, 2H), 2.83-2.70 (m, 4H), 2.50 (s, 3H), 2.46-2.22 (m, 16H), 2.20-2.08 (m, 4H), 1.89-1.82 (m, 2H), 1.80-1.71 (m, 3H), 1.66 (dt, J=14.0, 7.0 Hz, 2H), 1.48-1.40 (m, 3H), 1.38 (dd, J=11.1, 4.7 Hz, 1H), 1.25 (s, 10H), 1.08-1.02 (m, 9H), 0.91 (s, 3H).

### Example 28: Preparation of Degraders #24-26

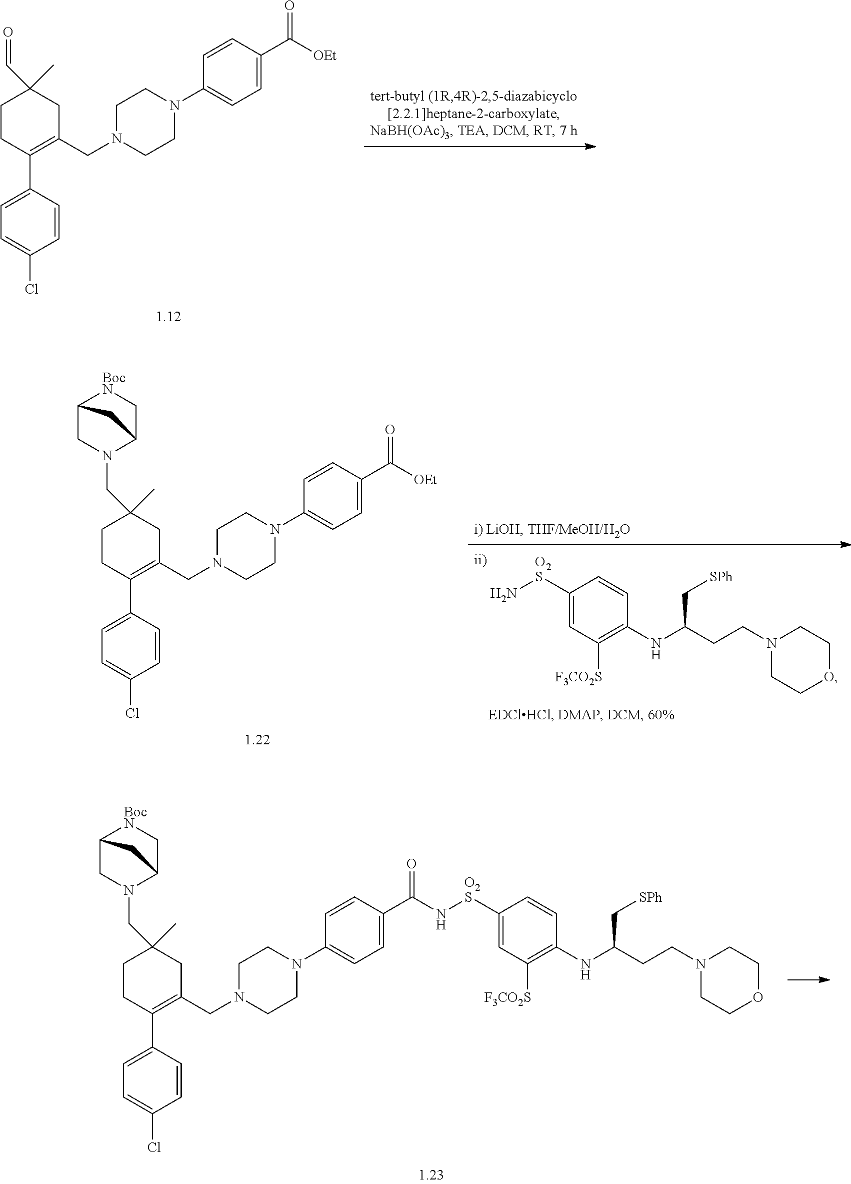

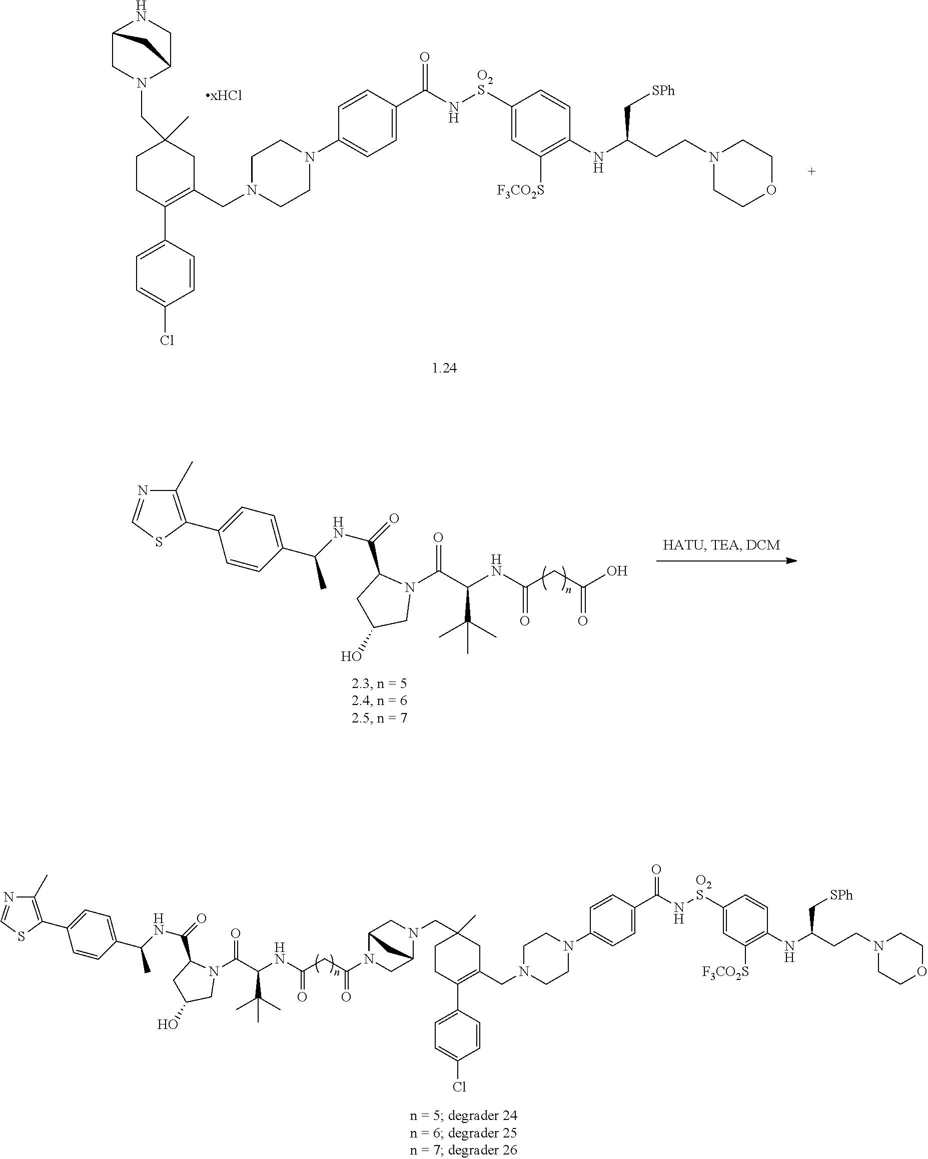

Preparation of tert-butyl (1R,4R)-5-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)-2,5-diazabicyclo[2.2.1]heptane-2-carboxylate (1.22): To a stirring solution of aldehyde 1.12 (1 equiv.) in DCM was added tert-butyl (1R,4R)-2,5-diazabicyclo [2.2.1] heptane-2-carboxylate (1.5 equiv.), NaBH(OAc)₃ (7 equiv.), and TEA (10 equiv.). The resulting mixture was stirred at room temperature for 8 h. After the completion of the reaction, the reaction mixture was diluted with DCM and then washed with water followed by brine. The organic portion was dried over anhydrous MgSO₄, filtered, and then concentrated under reduced pressure. The crude product was purified by silica gel column chromatography to afford the title compound. ¹H NMR (600 MHz, CDCl₃) δ 7.89 (d, J=8.9 Hz, 2H), 7.26 (d, J=10.2 Hz, 2H), 6.99 (d, J=8.4 Hz, 2H), 6.81 (d, J=9.0 Hz, 2H), 4.32 (q, J=7.2 Hz, 2H), 4.32-4.21 (m, 1H), 3.48 (m, 1H), 3.40-3.33 (m, 1H), 3.25 (s, 4H), 3.17 (m, 1H), 3.12-2.99 (m, 1H), 2.80 (s, 2H), 2.70-2.54 (m, 1H), 2.46 (s, 2H), 2.35 (d, J=5.7 Hz, 4H), 2.23 (d, J=27.3 Hz, 2H), 2.11 (m, 1H), 1.92 (d, J=18.5 Hz, 1H), 1.80 (s, 1H), 1.69 (s, 3H), 1.56 (s, 2H), 1.46 (s, 9H), 1.42 (s, 1H), 0.92 (s, 3H). ESI⁺, m/z [M+H]⁺=663.3.

Preparation of tert-butyl (1R,4R)-5-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,5-diazabicyclo[2.2.1]heptane-2-carboxylate (1.23): Compound 1.23 was prepared from compound 1.22 following the same procedure as compound 1.9 was prepared from the compound 1.8. ¹H NMR (600 MHz, CDCl₃) δ 8.35 (s, 1H), 8.10 (d, J=9.2 Hz, 1H), 7.68-7.61 (m, 2H), 7.37 (d, J=7.5 Hz, 2H), 7.30 (t, J=7.2 Hz, 2H), 7.27 (s, 2H), 7.09-7.01 (m, 1H), 7.01-6.95 (m, 2H), 6.76 (d, J=8.2 Hz, 2H), 6.59 (d, J=9.3 Hz, 1H), 4.34 (s, 0.5H), 4.22 (s, 0.5H), 3.89 (s, 1H), 3.65 (s, 4H), 3.55-3.47 (m, 1H), 3.41 (d, J=8.9 Hz, 1H), 3.27 (s, 4H), 3.20-3.13 (m, 1H), 3.10 (dd, J=13.8, 5.0 Hz, 1H), 3.02 (dd, J=13.8, 7.2 Hz, 1H), 2.83 (s, 2H), 2.71-2.56 (m, 1H), 2.46 (d, J=28.9 Hz, 4H), 2.40-2.34 (m, 4H), 2.32 (d, J=9.7 Hz, 3H), 2.27 (d, J=8.6 Hz, 1H), 2.22 (s, 1H), 2.12 (d, J=5.7 Hz, 3H), 1.93 (d, J=15.4 Hz, 2H), 1.82 (d, J=13.5 Hz, 2H), 1.73-1.62 (m, 3H), 1.46 (s, 9H), 0.93 (s, 3H). ESI⁺, m/z [M+H]⁺=1170.5.

Preparation of 4-(4-((4-(((1R,4R)-2,5-diazabicyclo[2.2.1]heptan-2-yl)methyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (1.24): Compound 1.24 was prepared from compound 1.23 following the same procedure as compound 1.10 was prepared from compound 1.9. ESI⁺, m/z [M+H]⁺=1070.3.

General procedure for the preparation of degraders #24-26: Degraders #24-26 was prepared following the same procedure as degrader 1 was prepared with amine 1.24 in place of amine 1.10.

(2S,4R)-1-((2S)-2-(7-((1R,4R)-5-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,5-diazabicyclo[2.2.1]heptan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (degrader #24): ¹H NMR (600 MHz, CDCl₃) δ 8.68 (s, 1H), 8.34-8.30 (m, 1H), 8.12 (t, J=7.9 Hz, 1H), 7.74 (t, J=9.3 Hz, 1H), 7.69 (d, J=7.9 Hz, 1H), 7.39 (ddd, J=15.0, 6.8, 2.1 Hz, 6H), 7.26 (s, 5H), 7.04 (t, J=7.3 Hz, 1H), 6.98 (dd, J=8.3, 4.8 Hz, 2H), 6.81-6.73 (m, 2H), 6.60 (dd, J=9.2, 6.1 Hz, 1H), 6.33 (dd, J=8.1, 5.0 Hz, 1H), 5.14-5.04 (m, 1H), 4.80-4.61 (m, 3H), 4.49 (s, 1H), 4.24-4.08 (m, 1H), 3.93-3.84 (m, 1H), 3.70-3.62 (m, 4H), 3.62-3.37 (m, 3H), 3.25 (d, J=23.8 Hz, 5H), 3.14-3.07 (m, 1H), 3.01 (dd, J=13.8, 7.2 Hz, 1H), 2.84 (d, J=18.8 Hz, 2H), 2.50 (d, J=3.5 Hz, 3H), 2.43-2.21 (m, 14H), 2.15-2.06 (m, 4H), 1.98-1.74 (m, 6H), 1.72-1.60 (m, 5H), 1.45 (dd, J=28.0, 6.9 Hz, 4H), 1.32 (d, J=15.2 Hz, 7H), 1.08-1.02 (m, 9H), 0.94-0.88 (m, 3H).

(2S,4R)-1-((2S)-2-(8-((1R,4R)-5-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,5-diazabicyclo[2.2.1]heptan-2-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #25): ¹H NMR (600 MHz, CDCl₃) δ 8.67 (s, 1H), 8.32 (dd, J=7.4, 1.8 Hz, 1H), 8.12-8.07 (m, 1H), 7.77-7.70 (m, 2H), 7.46 (dt, J=14.7, 8.1 Hz, 1H), 7.41-7.31 (m, 6H), 7.31-7.23 (m, 5H), 7.04-6.99 (m, 1H), 6.97 (d, J=7.5 Hz, 2H), 6.75 (dt, J=6.5, 4.0 Hz, 2H), 6.60 (d, J=9.4 Hz, 1H), 6.48-6.29 (m, 1H), 5.08 (dt, J=14.7, 7.3 Hz, 1H), 4.69 (dtd, J=24.3, 19.0, 17.6, 8.6 Hz, 3H), 4.49 (s, 1H), 4.23-4.09 (m, 1H), 3.89 (dt, J=8.1, 4.3 Hz, 1H), 3.64 (dd, J=11.7, 5.5 Hz, 4H), 3.62-3.41 (m, 3H), 3.30-3.13 (m, 5H), 3.09 (dd, J=13.9, 5.0 Hz, 1H), 3.01 (dd, J=13.9, 7.2 Hz, 1H), 2.94-2.58 (m, 3H), 2.50 (s, 3H), 2.48-2.27 (m, 13H), 2.28-2.08 (m, 9H), 1.95-1.79 (m, 1H), 1.74-1.53 (m, 6H), 1.50-1.43 (m, 3H), 1.42-1.18 (m, 9H), 1.07-1.01 (m, 9H), 0.92-0.87 (m, 3H).

(2S,4R)-1-((2S)-2-(9-((1R,4R)-5-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,5-diazabicyclo[2.2.1]heptan-2-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide (degrader #26): ¹H NMR (600 MHz, CDCl₃) δ 8.67 (d, J=1.4 Hz, 1H), 8.31 (dd, J=5.9, 3.0 Hz, 1H), 8.11 (d, J=9.2 Hz, 1H), 7.76-7.67 (m, 2H), 7.53-7.33 (m, 6H), 7.32-7.23 (m, 5H), 7.03 (dd, J=7.7, 4.4 Hz, 1H), 6.97 (dd, J=8.3, 2.0 Hz, 2H), 6.78-6.72 (m, 2H), 6.64-6.56 (m, 1H), 6.48-6.25 (m, 1H), 5.09 (ddd, J=28.6, 13.4, 7.3 Hz, 1H), 4.79-4.63 (m, 3H), 4.49 (s, 1H), 4.26-4.07 (m, 2H), 3.89 (s, 1H), 3.64 (dd, J=11.7, 5.5 Hz, 4H), 3.62-3.39 (m, 3H), 3.31-3.18 (m, 5H), 3.09 (dd, J=13.8, 4.9 Hz, 1H), 3.01 (dd, J=13.8, 7.2 Hz, 1H), 2.92-2.61 (m, 3H), 2.51-2.47 (m, 3H), 2.47-2.27 (m, 13H), 2.27-2.03 (m, 9H), 1.93-1.50 (m, 8H), 1.49-1.33 (m, 4H), 1.25 (s, 8H), 1.05 (d, J=2.8 Hz, 9H), 0.91 (dd, J=10.4, 3.0 Hz, 3H).

### Example 29: Preparation of Degraders #27-29

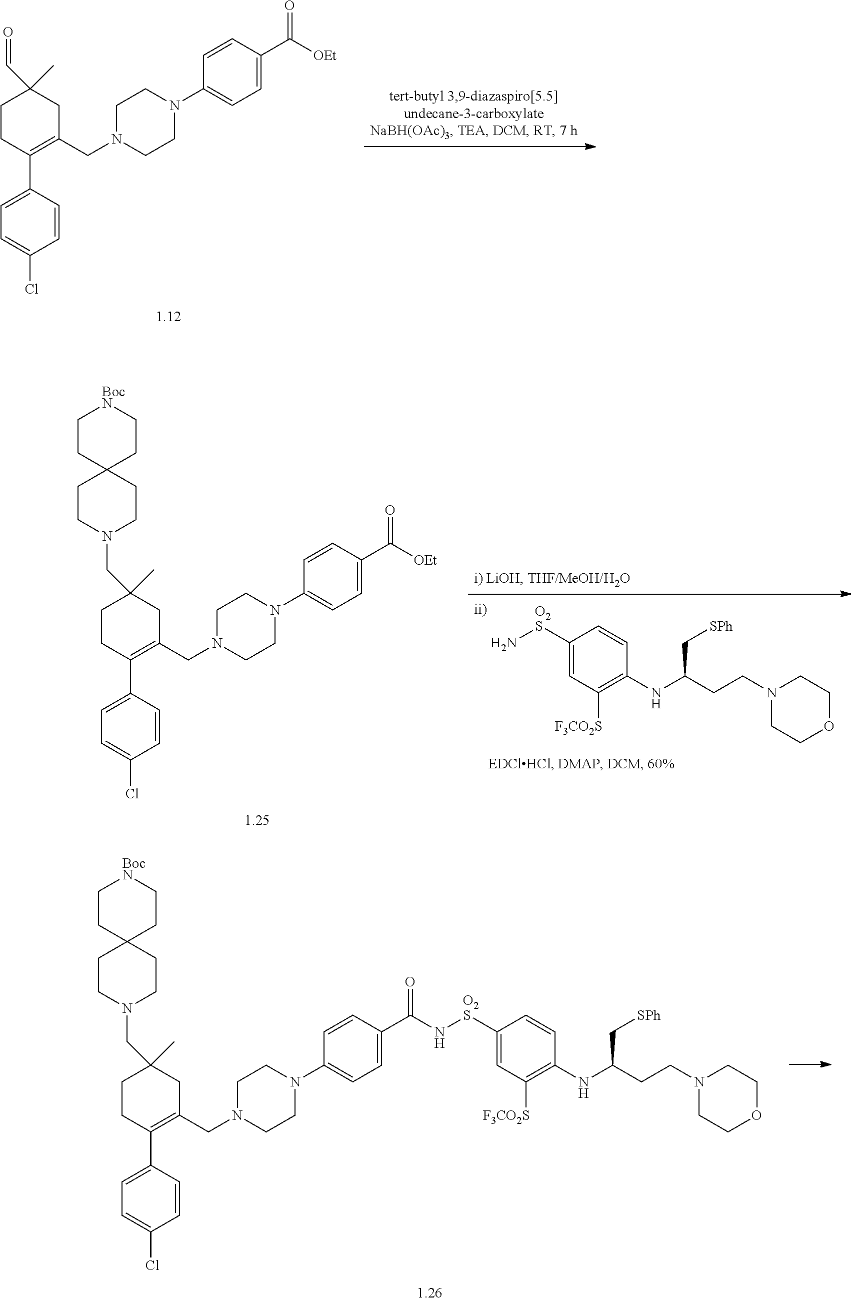

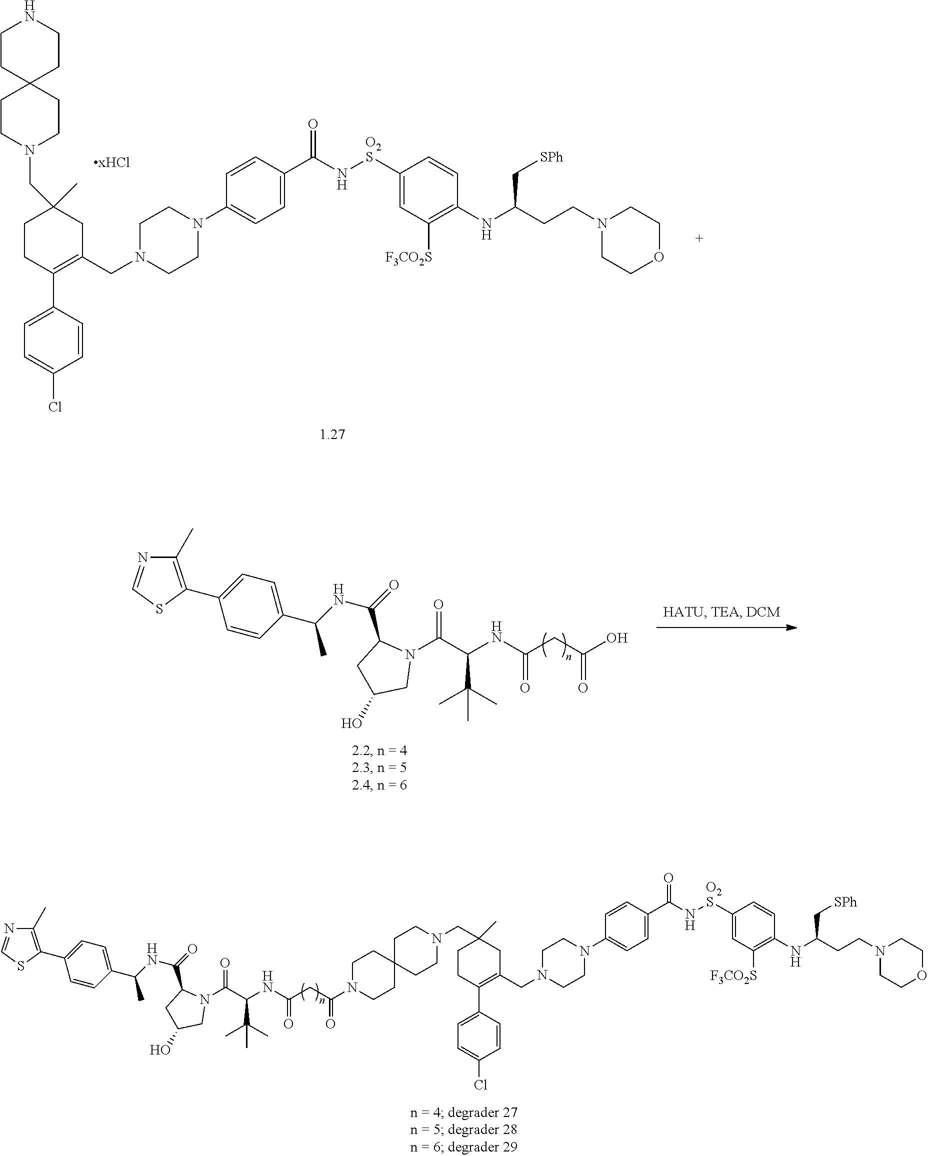

Preparation of tert-butyl 9-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)-3,9-diazaspiro[5.5]undecane-3-carboxylate (1.25): To a stirring solution of aldehyde 1.12 (1 equiv.) in DCM was added tert-butyl (1R,4R)-2,5-diazabicyclo [2.2.1]heptane-2-carboxylate (1.5 equiv.), NaBH(OAc)₃ (7 equiv.) and TEA (10 equiv.). The resulting mixture was stirred at room temperature for 7 h. After the completion of the reaction, the reaction mixture was diluted with DCM and then washed with water followed by brine. The organic portion was dried over anhydrous MgSO₄, filtered, and concentrated under reduced pressure. The crude product was purified by silica gel column chromatography to afford the title compound. ¹H NMR (600 MHz, CDCl₃) δ 7.89 (d, J=9.0 Hz, 2H), 7.26 (d, J=9.0 Hz, 2H), 6.99 (d, J=8.5 Hz, 2H), 6.81 (d, J=9.0 Hz, 2H), 4.32 (q, J=7.1 Hz, 2H), 3.35 (t, J=5.8 Hz, 4H), 3.25 (t, J=5.2 Hz, 4H), 2.79 (s, 2H), 2.53-2.45 (m, 4H), 2.35 (ddq, J=16.2, 11.0, 4.9 Hz, 4H), 2.27 (dd, J=16.8, 8.8 Hz, 1H), 2.21 (d, J=5.7 Hz, 1H), 2.18 (d, J=4.6 Hz, 2H), 2.12 (d, J=17.4 Hz, 1H), 1.90 (d, J=17.3 Hz, 1H), 1.60 (d, J=8.8 Hz, 2H), 1.50-1.47 (m, 4H), 1.45 (s, 9H), 1.41 (s, 4H), 1.36 (t, J=7.1 Hz, 3H), 0.93 (s, 3H). ESI⁺, m/z [M+H]⁺=719.4.

Preparation of tert-butyl 9-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-3,9-diazaspiro[5.5]undecane-3-carboxylate (1.26): Compound 1.26 was prepared from compound 1.25 following the same procedure as compound 1.9 was prepared from the compound 1.8. ¹H NMR (600 MHz, CDCl₃) δ 8.34 (d, J=1.6 Hz, 1H), 8.10 (d, J=8.1 Hz, 1H), 7.72 (d, J=8.4 Hz, 2H), 7.33 (t, J=9.0 Hz, 4H), 7.24 (d, J=7.7 Hz, 2H), 7.20 (t, J=7.3 Hz, 1H), 7.05 (d, J=6.9 Hz, 2H), 6.92 (s, 1H), 6.78 (d, J=8.3 Hz, 2H), 6.74 (d, J=9.0 Hz, 1H), 4.14-3.83 (m, 6H), 3.76 (s, 2H), 3.69 (d, J=12.3 Hz, 2H), 3.63-3.49 (m, 3H), 3.42 (d, J=12.6 Hz, 1H), 3.39-3.24 (m, 4H), 3.23-3.10 (m, 4H), 3.02 (dt, J=22.6, 14.9 Hz, 3H), 2.90 (dd, J=16.8, 9.2 Hz, 2H), 2.63-2.53 (m, 1H), 2.47 (dd, J=38.2, 16.6 Hz, 3H), 2.34 (dd, J=21.2, 9.9 Hz, 4H), 2.04 (s, 11H), 1.75-1.62 (m, 2H), 1.42 (s, 9H), 1.10 (s, 3H). ESI⁺, m/z [M+H]⁺=1225.9.

Preparation of 4-(4-((4-((3,9-diazaspiro[5.5]undecan-3-yl)methyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (1.27): Compound 1.27 was prepared from compound 1.26 following the same procedure as compound 1.10 was prepared from compound 1.9. ESI⁺, m/z [M+H]⁺=1125.6.

General procedure for the preparation of degraders #27-29: Degraders #27-29 was prepared by following the same procedure for the preparation of degrader 1 with amine 1.27 in place of amine 1.10.

(2S,4R)-1-((2S)-2-(6-(9-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-3,9-diazaspiro[5.5]undecan-3-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (degrader #27): ¹H NMR (600 MHz, CDCl₃) δ 8.67 (s, 1H), 8.34-8.32 (m, 1H), 8.05 (d, J=9.1 Hz, 1H), 7.76 (d, J=8.4 Hz, 2H), 7.49-7.33 (m, 7H), 7.32-7.23 (m, 5H), 7.00 (d, J=7.9 Hz, 2H), 6.93 (d, J=7.9 Hz, 1H), 6.68 (s, 2H), 6.58 (d, J=9.2 Hz, 1H), 6.47 (d, J=8.9 Hz, 1H), 5.13-5.04 (m, 1H), 4.78-4.71 (m, 1H), 4.58 (d, J=8.6 Hz, 1H), 4.48 (s, 1H), 4.07 (d, J=11.1 Hz, 1H), 3.91-3.82 (m, 1H), 3.69-3.62 (m, 4H), 3.58 (dd, J=11.2, 3.3 Hz, 1H), 3.46 (s, 2H), 3.27 (d, J=40.8 Hz, 6H), 3.14-3.07 (m, 3H), 3.01-2.97 (m, 1H), 2.86-2.63 (m, 4H), 2.51 (s, 3H), 2.50-2.34 (m, 9H), 2.34-2.24 (m, 9H), 2.23-2.17 (m, 3H), 2.15-2.07 (m, 5H), 1.65 (dt, J=17.3, 8.7 Hz, 4H), 1.49-1.36 (m, 11H), 1.04 (s, 12H).

(2S,4R)-1-((2S)-2-(7-(9-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-3,9-diazaspiro[5.5]undecan-3-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (degrader #28): ¹H NMR (600 MHz, CDCl₃) δ 8.66 (s, 1H), 8.33 (d, J=1.9 Hz, 1H), 8.04 (d, J=9.1 Hz, 1H), 7.78 (d, J=8.5 Hz, 2H), 7.47-7.33 (m, 7H), 7.31-7.22 (m, 5H), 6.99 (d, J=8.0 Hz, 2H), 6.90 (d, J=8.1 Hz, 1H), 6.65 (s, 2H), 6.58 (d, J=9.3 Hz, 1H), 6.33 (t, J=9.6 Hz, 1H), 5.07 (td, J=7.2, 3.0 Hz, 1H), 4.73 (td, J=7.9, 3.7 Hz, 1H), 4.59 (d, J=8.8 Hz, 1H), 4.48 (s, 1H), 4.06 (d, J=11.3 Hz, 1H), 3.91-3.84 (m, 1H), 3.65 (h, J=7.9 Hz, 4H), 3.59 (dd, J=11.2, 3.4 Hz, 1H), 3.45 (s, 2H), 3.27 (d, J=42.6 Hz, 6H), 3.12 (ddd, J=28.6, 14.2, 6.1 Hz, 3H), 2.99 (dd, J=13.8, 7.3 Hz, 1H), 2.89-2.70 (m, 4H), 2.69-2.53 (m, 4H), 2.51 (s, 3H), 2.49-2.19 (m, 17H), 2.19-2.05 (m, 4H), 1.61 (dtd, J=44.9, 14.4, 7.7 Hz, 9H), 1.50-1.38 (m, 9H), 1.03 (s, 12H).

(2S,4R)-1-((2S)-2-(8-(9-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-3,9-diazaspiro[5.5]undecan-3-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #29): ¹H NMR (600 MHz, CDCl₃) δ 8.66 (s, 1H), 8.32 (d, J=1.7 Hz, 1H), 8.04 (d, J=9.0 Hz, 1H), 7.78 (d, J=8.4 Hz, 2H), 7.49 (t, J=7.0 Hz, 1H), 7.40-7.35 (m, 6H), 7.30-7.22 (m, 5H), 6.99 (d, J=8.0 Hz, 2H), 6.91 (d, J=8.3 Hz, 1H), 6.67-6.64 (m, 1H), 6.57 (d, J=9.4 Hz, 1H), 6.30 (dd, J=8.5, 4.9 Hz, 1H), 5.07 (p, J=6.9 Hz, 1H), 4.72 (t, J=7.9 Hz, 1H), 4.59 (d, J=8.9 Hz, 1H), 4.49 (s, 1H), 4.08 (d, J=11.2 Hz, 1H), 3.91-3.84 (m, 1H), 3.64 (q, J=14.2, 10.4 Hz, 4H), 3.58 (dd, J=11.3, 3.5 Hz, 1H), 3.46 (s, 2H), 3.31 (s, 3H), 3.27-3.13 (m, 5H), 3.09 (dd, J=13.8, 4.9 Hz, 2H), 2.99 (dd, J=13.8, 7.3 Hz, 1H), 2.91-2.69 (m, 5H), 2.69-2.53 (m, 5H), 2.51 (s, 3H), 2.48-2.39 (m, 5H), 2.39-2.05 (m, 15H), 1.71-1.51 (m, 8H), 1.46 (d, J=6.9 Hz, 6H), 1.29 (s, 5H), 1.03 (s, 12H).

### Example 30: Preparation of Degraders #30-32

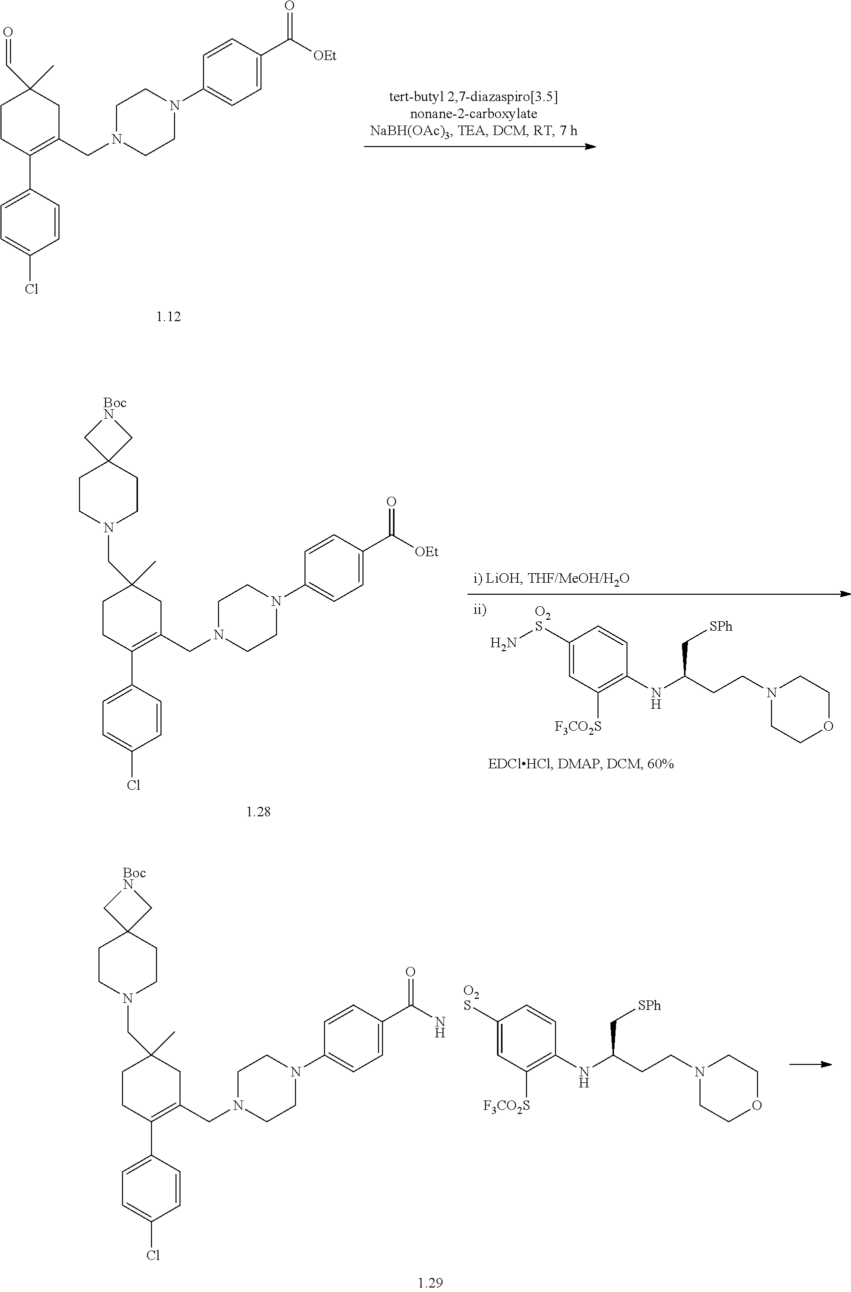

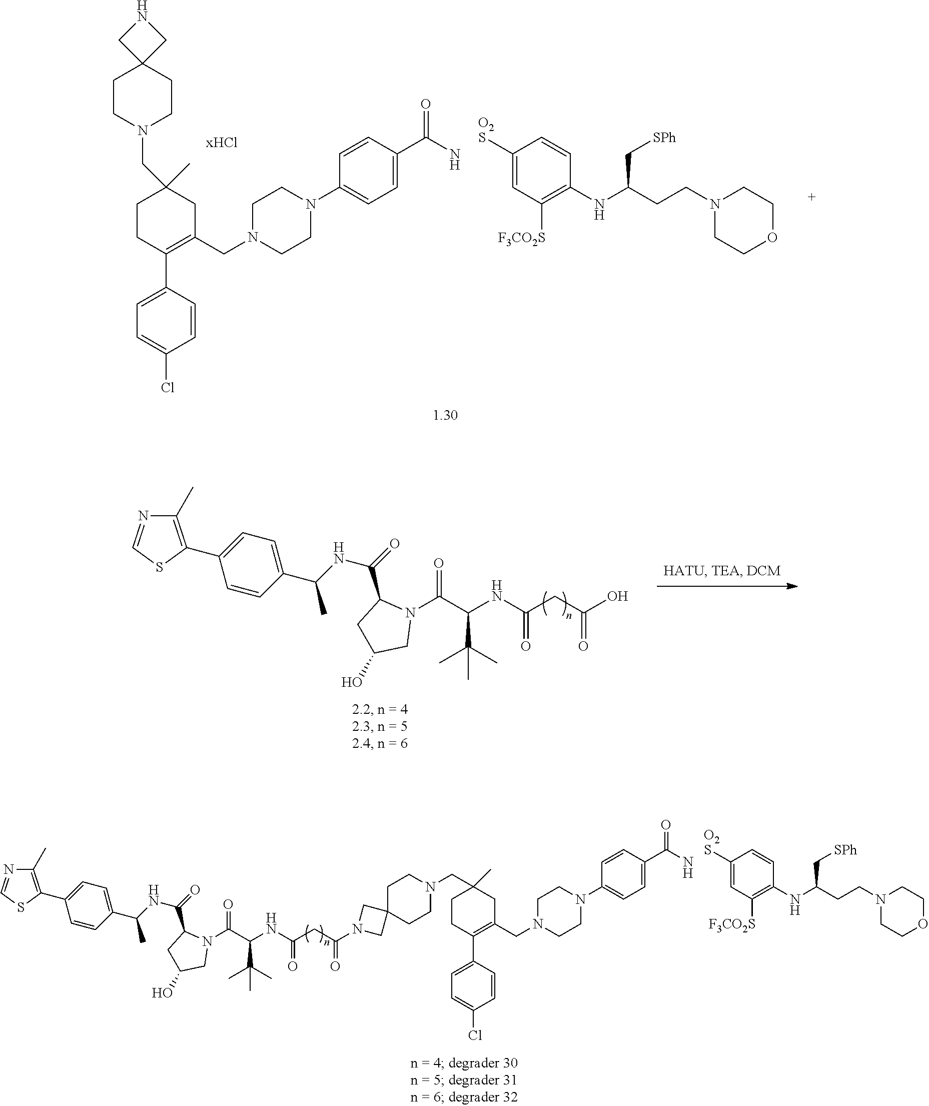

Preparation of tert-butyl 7-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)-2,7-diazaspiro[3.5]nonane-2-carboxylate (1.28): To a stirring solution of aldehyde 1.12 (1 equiv.) in DCM was added tert-butyl 2,7-diazaspiro[3.5]nonane-2-carboxylate (1.5 equiv.), NaBH(OAc)₃ (7 equiv.) and TEA (10 equiv.). The resulting mixture was stirred at room temperature for 7 h. After the completion of the reaction, the reaction mixture was diluted with DCM and then washed with water followed by brine solution. The organic portion was dried over anhydrous MgSO₄, filtered, and concentrated under reduced pressure. The crude product was purified by silica gel column chromatography to afford the title compound. ¹H NMR (600 MHz, CDCl₃) δ 7.90 (d, J=9.0 Hz, 2H), 7.26 (d, J=9.0 Hz, 2H), 6.99 (d, J=8.4 Hz, 2H), 6.81 (d, J=9.1 Hz, 2H), 4.32 (q, J=7.1 Hz, 2H), 3.59 (s, 4H), 3.24 (t, J=5.1 Hz, 4H), 2.79 (s, 2H), 2.44 (s, 4H), 2.38-2.31 (m, 4H), 2.23 (d, J=27.5 Hz, 2H), 2.15 (d, J=3.4 Hz, 2H), 2.11 (d, J=18.3 Hz, 1H), 1.90 (d, J=17.2 Hz, 1H), 1.72 (t, J=5.5 Hz, 4H), 1.60 (m, 2H), 1.44 (s, 9H), 1.36 (t, J=7.1 Hz, 3H), 0.92 (s, 3H). ESI⁺, m/z[M+H]⁺=691.4.

Preparation of tert-butyl 7-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,7-diazaspiro[3.5]nonane-2-carboxylate (1.29): Compound 1.29 was prepared from compound 1.28 following the same procedure as compound 1.9 was prepared from the compound 1.8. ¹H NMR (600 MHz, CDCl₃) δ 8.35 (d, J=2.3 Hz, 1H), 8.09 (dd, J=9.2, 2.3 Hz, 1H), 7.66 (d, J=8.5 Hz, 2H), 7.40-7.35 (m, 2H), 7.30 (t, J=7.4 Hz, 2H), 7.26 (s, 3H), 7.04 (d, J=8.5 Hz, 1H), 6.97 (d, J=8.4 Hz, 2H), 6.76 (d, J=8.6 Hz, 2H), 6.59 (d, J=9.3 Hz, 1H), 3.89 (d, J=9.7 Hz, 1H), 3.66 (d, J=3.4 Hz, 4H), 3.58 (s, 4H), 3.25 (t, J=5.2 Hz, 4H), 3.10 (dd, J=13.9, 5.0 Hz, 1H), 3.01 (dd, J=13.9, 7.3 Hz, 1H), 2.84 (s, 2H), 2.45 (d, J=21.7 Hz, 7H), 2.37 (dd, J=12.7, 6.3 Hz, 6H), 2.32 (s, 3H), 2.26 (s, 1H), 2.25-2.16 (m, 4H), 2.13 (d, J=15.1 Hz, 3H), 1.93 (d, J=17.0 Hz, 2H), 1.68-1.61 (m, 1H), 1.59 (d, J=6.5 Hz, 1H), 1.43 (s, 10H), 0.93 (s, 3H). ESI⁺, m/z [M+H]⁺=1197.9.

Preparation of 4-(4-((4-((2,7-diazaspiro[3.5]nonan-7-yl)methyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (1.30): Compound 1.30 was prepared from compound 1.29 following the same procedure as compound 1.10 was prepared from compound 1.9. ESI⁺, m/z [M+H]⁺=1097.8.

General procedure for the preparation of degraders #30-32: Degraders #30-32 was prepared following the same procedure as DEGRADER 1 was prepared, only amine 1.30 was taken in place amine 1.10.

(2S,4R)-1-((2S)-2-(6-(7-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,7-diazaspiro[3.5]nonan-2-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (degrader #30): ¹H NMR (600 MHz, CDCl₃) δ 8.67 (s, 1H), 8.33 (s, 1H), 8.09 (d, J=8.6 Hz, 1H), 7.70 (d, J=6.5 Hz, 2H), 7.47-7.32 (m, 7H), 7.31-7.22 (m, 5H), 7.00 (dd, J=17.6, 8.1 Hz, 3H), 6.73 (d, J=6.6 Hz, 2H), 6.60 (d, J=9.4 Hz, 1H), 6.50 (dd, J=21.5, 8.6 Hz, 1H), 5.08 (h, J=6.6 Hz, 1H), 4.74 (t, J=7.2 Hz, 1H), 4.63-4.58 (m, 1H), 4.47 (s, 1H), 4.09 (d, J=11.1 Hz, 1H), 3.94-3.87 (m, 1H), 3.76-3.55 (m, 9H), 3.24 (s, 4H), 3.10 (dd, J=13.8, 4.9 Hz, 1H), 3.01 (dd, J=13.8, 7.2 Hz, 1H), 2.96-2.63 (m, 4H), 2.62-2.51 (m, 3H), 2.51 (s, 3H), 2.47-2.29 (m, 13H), 2.15 (ddd, J=72.6, 34.1, 13.1 Hz, 12H), 1.70-1.52 (m, 8H), 1.45 (q, J=12.0, 9.3 Hz, 4H), 1.04 (s, 9H), 0.95 (s, 3H).

(2S,4R)-1-((2S)-2-(7-(7-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,7-diazaspiro[3.5]nonan-2-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (degrader #31): ¹H NMR (600 MHz, CDCl₃) δ 8.67 (s, 1H), 8.33 (s, 1H), 8.08 (d, J=8.8 Hz, 1H), 7.71 (d, J=5.8 Hz, 2H), 7.44-7.32 (m, 7H), 7.32-7.22 (m, 5H), 6.99 (t, J=9.8 Hz, 3H), 6.76-6.68 (m, 2H), 6.60 (d, J=9.3 Hz, 1H), 6.35 (dd, J=36.7, 8.7 Hz, 1H), 5.07 (h, J=7.1 Hz, 1H), 4.73 (q, J=7.7 Hz, 1H), 4.62 (t, J=8.5 Hz, 1H), 4.48 (s, 1H), 4.07 (d, J=11.2 Hz, 1H), 3.92-3.86 (m, 1H), 3.73 (q, J=8.7 Hz, 2H), 3.69-3.57 (m, 8H), 3.24 (s, 4H), 3.10 (dd, J=13.8, 4.9 Hz, 1H), 3.01 (dd, J=13.8, 7.2 Hz, 1H), 2.95-2.52 (m, 8H), 2.50 (s, 3H), 2.45-2.24 (m, 14H), 2.08 (dddt, J=55.2, 45.6, 27.6, 10.4 Hz, 9H), 1.65 (s, 3H), 1.57 (dt, J=12.2, 6.4 Hz, 4H), 1.45 (dd, J=12.9, 6.9 Hz, 4H), 1.31 (s, 3H), 1.03 (s, 9H), 0.95 (s, 3H).

(2S,4R)-1-((2S)-2-(8-(7-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,7-diazaspiro[3.5]nonan-2-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #32): ¹H NMR (600 MHz, CDCl₃) δ 8.66 (s, 1H), 8.32 (s, 1H), 8.07 (d, J=9.2 Hz, 1H), 7.73 (d, J=7.6 Hz, 2H), 7.48-7.44 (m, 1H), 7.41-7.34 (m, 6H), 7.32-7.22 (m, 5H), 6.98 (d, J=8.2 Hz, 3H), 6.70 (d, J=6.6 Hz, 2H), 6.59 (d, J=9.4 Hz, 1H), 6.31 (t, J=8.3 Hz, 1H), 5.10-5.04 (m, 1H), 4.71 (td, J=7.8, 2.2 Hz, 1H), 4.62-4.57 (m, 1H), 4.48 (s, 1H), 4.08 (d, J=11.3 Hz, 1H), 3.92-3.83 (m, 1H), 3.73 (s, 2H), 3.70-3.56 (m, 8H), 3.25 (s, 4H), 3.09 (dd, J=13.8, 4.9 Hz, 1H), 3.01 (dd, J=13.8, 7.2 Hz, 1H), 2.93 (s, 1H), 2.86-2.51 (m, 4H), 2.50 (s, 3H), 2.49-2.24 (m, 14H), 2.24-1.96 (m, 8H), 1.89-1.49 (m, 9H), 1.44 (dd, J=15.9, 6.6 Hz, 4H), 1.30 (dd, J=44.5, 9.2 Hz, 7H), 1.03 (s, 9H), 0.95 (s, 3H).

### Example 31: Preparation of Degraders #33-35

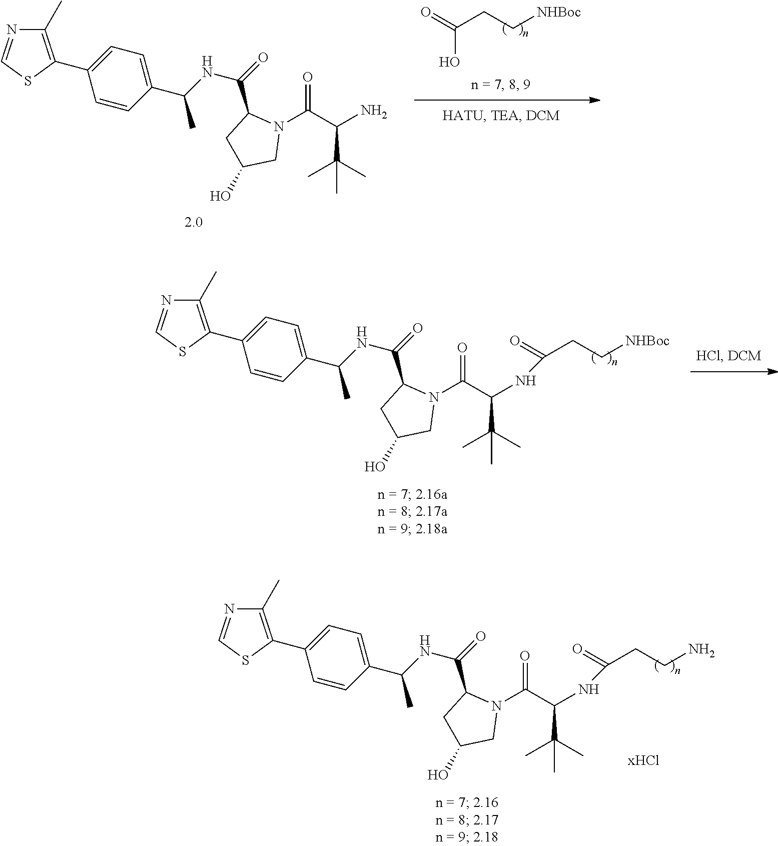

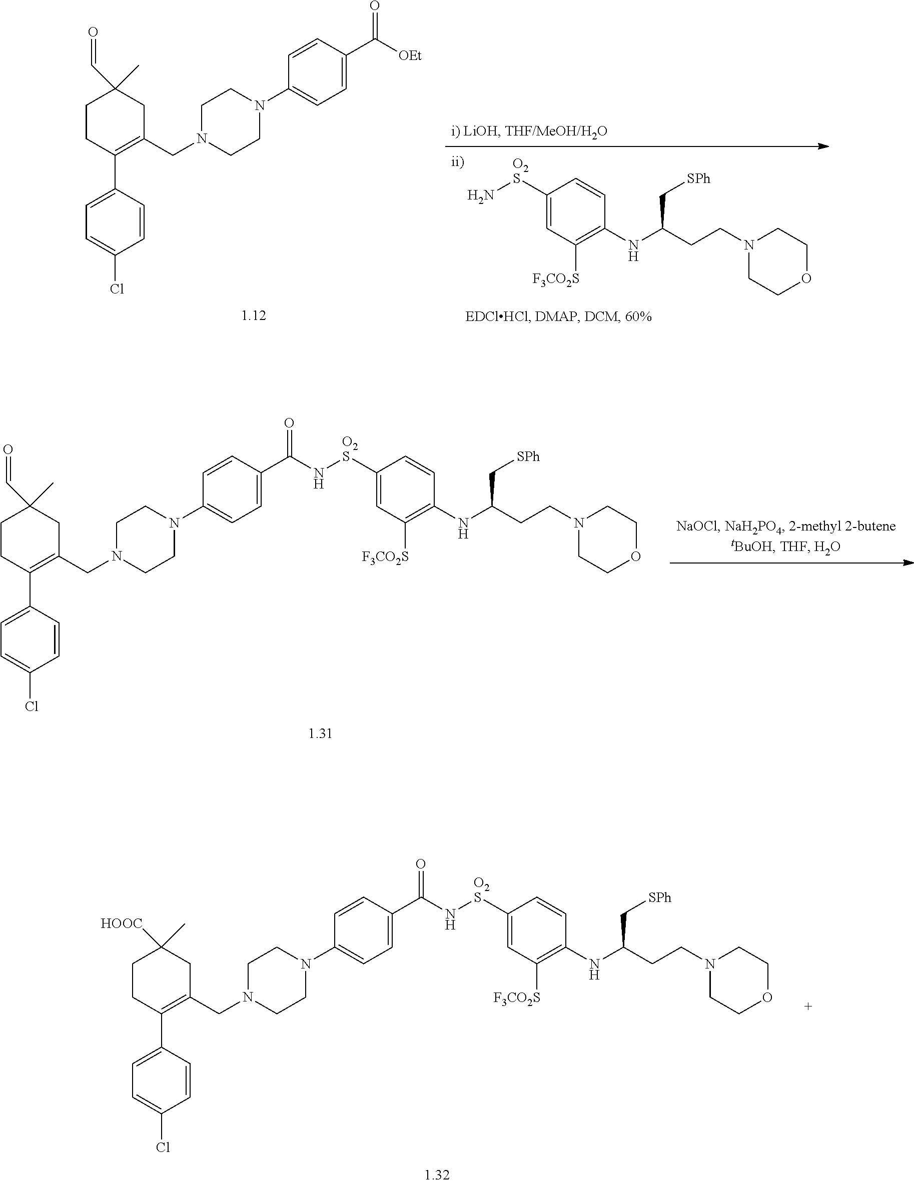

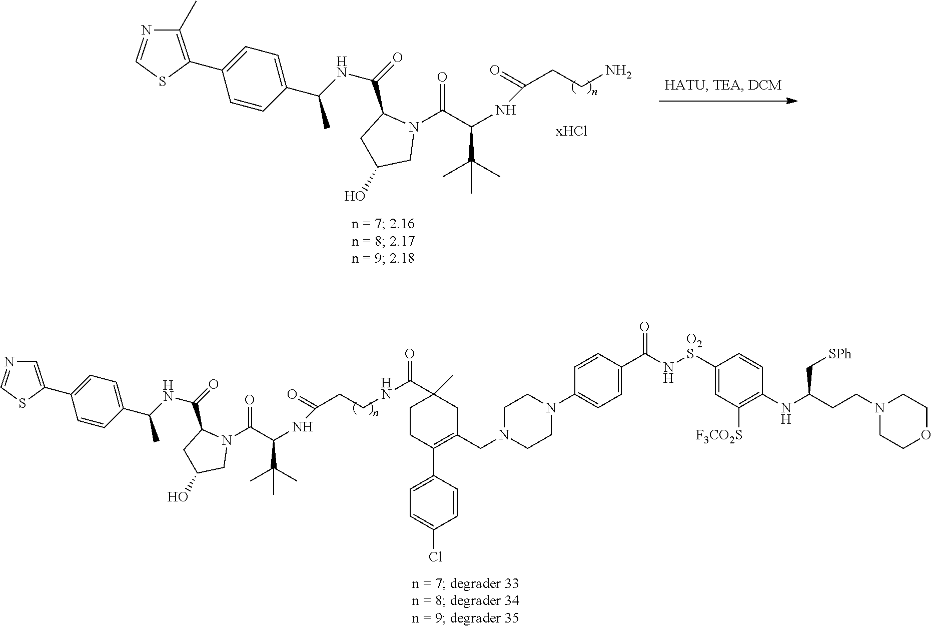

General procedure for the preparation of compounds 2.16-2.18: A mixture of amine 2.0 (1.0 equiv.), mono-protected amino acids (1.1 equiv.), HATU (1.2 equiv.), and TEA (5 equiv.) was taken in DCM and the reaction mixture was stirred at room temperature for 4 h. After completion of the reaction, DCM was evaporated, and the crude was directly charged to the column purification to afford compounds 2.16a-2.18a. The Boc group on 2.16a-2.18a was deprotected using HCl in DCM to afford the title compound 2.16-2.18, which were used in the next step without further purification.

tert-butyl (9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononyl)carbamate (2.16a): ¹H NMR (600 MHz, CDCl₃) δ 8.68 (s, 1H), 7.43 (d, J=7.2 Hz, 1H), 7.41 (d, J=8.3 Hz, 2H), 7.37 (d, J=8.2 Hz, 2H), 6.13 (d, J=7.7 Hz, 1H), 5.08 (p, J=7.0 Hz, 1H), 4.73 (t, J=8.0 Hz, 1H), 4.55 (d, J=8.7 Hz, 2H), 4.51 (s, 1H), 4.14 (d, J=11.5 Hz, 1H), 3.58 (dd, J=11.4, 3.5 Hz, 1H), 3.12-3.06 (m, 2H), 2.58-2.53 (m, 1H), 2.53 (s, 3H), 2.21 (td, J=7.3, 2.4 Hz, 2H), 2.09 (dd, J=12.8, 8.5 Hz, 1H), 1.64-1.56 (m, 3H), 1.47 (d, J=6.9 Hz, 3H), 1.43 (s, 9H), 1.28 (s, 9H), 1.05 (s, 9H).

tert-butyl (10-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-10-oxodecyl)carbamate (2.17a): ¹H NMR (600 MHz, CDCl3) δ 8.67 (s, 1H), 7.43 (s, 1H), 7.41 (d, J=8.3 Hz, 2H), 7.36 (d, J=8.2 Hz, 2H), 6.11 (d, J=8.8 Hz, 1H), 5.08 (p, J=7.0 Hz, 1H), 4.74 (t, J=7.9 Hz, 1H), 4.55 (d, J=8.7 Hz, 1H), 4.52 (s, 2H), 4.15 (d, J=11.4 Hz, 1H), 3.58 (dd, J=11.4, 3.6 Hz, 1H), 3.12-3.04 (m, 2H), 2.95 (s, 1H), 2.57 (ddd, J=12.7, 7.5, 4.7 Hz, 1H), 2.53 (s, 3H), 2.21 (t, J=7.5 Hz, 2H), 2.10-2.05 (m, 1H), 1.63-1.57 (m, 3H), 1.47 (d, J=6.9 Hz, 3H), 1.43 (s, 9H), 1.27 (s, 11H), 1.05 (s, 9H).

tert-butyl (11-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-11-oxoundecyl)carbamate (2.18a): ¹H NMR (600 MHz, CDCl3) δ 8.67 (s, 1H), 7.43 (d, J=7.4 Hz, 1H), 7.41 (d, J=8.3 Hz, 2H), 7.36 (d, J=8.2 Hz, 2H), 6.11 (d, J=8.1 Hz, 1H), 5.08 (p, J=7.0 Hz, 1H), 4.74 (t, J=7.9 Hz, 1H), 4.54 (d, J=8.7 Hz, 1H), 4.52 (s, 2H), 4.15 (d, J=11.5 Hz, 1H), 3.58 (dd, J=11.4, 3.6 Hz, 1H), 3.08 (dd, J=11.7, 6.9 Hz, 2H), 2.57 (ddd, J=12.8, 7.4, 4.8 Hz, 1H), 2.53 (s, 3H), 2.21 (t, J=7.6 Hz, 2H), 2.09-2.05 (m, 1H), 1.65-1.56 (m, 3H), 1.47 (d, J=6.9 Hz, 3H), 1.44 (s, 9H), 1.30-1.24 (m, 13H), 1.05 (s, 9H).

Preparation of 4-(4-((4′-chloro-4-formyl-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (1.31): Compound 1.31 was prepared from compound 1.12 by following the same procedure as compound 1.9 was prepared from the compound 1.8. ¹H NMR (600 MHz, CDCl3) δ 9.52 (s, 1H), 8.36 (s, 1H), 8.11 (dd, J=9.2, 1.9 Hz, 1H), 7.63 (d, J=8.6 Hz, 2H), 7.37 (d, J=7.5 Hz, 2H), 7.31 (t, J=7.4 Hz, 2H), 7.27 (d, J=7.5 Hz, 2H), 7.08 (d, J=8.3 Hz, 1H), 6.94 (d, J=8.4 Hz, 2H), 6.79 (d, J=8.7 Hz, 2H), 6.61 (d, J=9.3 Hz, 1H), 3.91 (s, 1H), 3.65 (t, J=7.6 Hz, 4H), 3.29 (t, J=4.7 Hz, 4H), 3.10 (dd, J=13.9, 5.0 Hz, 1H), 3.02 (dd, J=13.8, 7.2 Hz, 1H), 2.85 (s, 2H), 2.66 (d, J=17.6 Hz, 1H), 2.45-2.37 (m, 6H), 2.36-2.26 (m, 7H), 2.12 (dd, J=12.9, 4.7 Hz, 1H), 2.00 (dd, J=13.6, 6.8 Hz, 1H), 1.65 (ddt, J=36.1, 13.7, 6.5 Hz, 3H), 1.14 (s, 3H). ESI⁺, m/z [M+H]⁺=988.3.

Preparation of 4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carboxylic acid (1.32): To a stirring solution of aldehyde 1.31 in tert-butanol/THF (1/1) was added 2-methyl-2-butene (20 equiv.). The reaction mixture was cooled to −5° C. and a solution of NaOCl (2.5 equiv.) and NaH₂PO₄ in water was added dropwise. After 30 min, the reaction mixture was diluted with EtOAc. The organic portion was washed with water and brine, dried over anhydrous Na₂SO₄, filtered, and concentrated under reduced pressure. The crude material was purified by flash chromatography to afford the title compound. ¹H NMR (600 MHz, CD₃OD) δ 8.19 (d, J=2.2 Hz, 1H), 7.93 (dd, J=9.2, 2.3 Hz, 1H), 7.72 (d, J=8.8 Hz, 2H), 7.29-7.24 (m, 4H), 7.15 (t, J=7.7 Hz, 2H), 7.11-7.07 (m, 1H), 7.00 (d, J=8.4 Hz, 2H), 6.79 (d, J=9.1 Hz, 2H), 6.73 (d, J=9.4 Hz, 1H), 3.94 (dd, J=8.8, 5.0 Hz, 1H), 3.57 (td, J=6.2, 3.4 Hz, 4H), 3.28 (d, J=5.3 Hz, 4H), 3.18 (dd, J=14.3, 5.8 Hz, 1H), 3.15-3.12 (m, 2H), 3.09 (dd, J=14.3, 5.8 Hz, 1H), 2.76-2.67 (m, 3H), 2.64-2.57 (m, 2H), 2.51-2.42 (m, 3H), 2.41-2.32 (m, 4H), 2.24 (d, J=18.3 Hz, 1H), 2.10-1.99 (m, 3H), 1.75-1.67 (m, 1H), 1.63-1.57 (m, 1H), 1.24 (s, 3H). ESI⁺, m/z [M+H]⁺=1004.3.

General procedure for the preparation of degraders #33-35: Degraders #33-35 were prepared by following the same procedure as that of degrader 1, where acid 1.32 was coupled with amines (2.16, 2.17 and 2.18).

(2S,4R)-1-((2S)-2-(10-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carboxamido)decanamido)-3,3-dimethylbutanoyl)-4-hydroxy-N—((S)-1-(4-(4-methylthiazol-5-yl)phenyl)ethyl)pyrrolidine-2-carboxamide (degrader #33): ¹H NMR (600 MHz, CDCl₃) δ 8.68 (s, 1H), 8.29 (d, J=1.7 Hz, 1H), 8.09 (dd, J=9.2, 1.7 Hz, 1H), 7.80 (d, J=8.8 Hz, 1H), 7.76 (d, J=8.8 Hz, 1H), 7.42-7.33 (m, 7H), 7.33-7.23 (m, 5H), 7.03 (d, J=8.0 Hz, 1H), 6.96 (d, J=8.1 Hz, 2H), 6.76-6.71 (m, 2H), 6.61 (dd, J=9.3, 4.0 Hz, 1H), 6.33 (d, J=8.9 Hz, 1H), 5.09 (p, J=7.3, 6.9 Hz, 1H), 4.79-4.70 (m, 2H), 4.51 (s, 1H), 4.18 (d, J=11.6 Hz, 1H), 3.95-3.86 (m, 1H), 3.66 (q, J=9.7, 6.0 Hz, 4H), 3.59 (d, J=11.0 Hz, 1H), 3.44-3.28 (m, 1H), 3.28-3.16 (m, 4H), 3.16-3.06 (m, 3H), 3.01 (dd, J=13.8, 7.2 Hz, 2H), 2.85-2.64 (m, 2H), 2.54-2.29 (m, 14H), 2.25-2.05 (m, 8H), 1.71-1.50 (m, 5H), 1.48 (d, J=6.8 Hz, 5H), 1.41 (dt, J=22.2, 7.4 Hz, 4H), 1.26 (d, J=4.6 Hz, 6H), 1.05 (s, 9H).

(2S,4R)-1-((2S)-2-(11-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carboxamido)undecanamido)-3,3-dimethylbutanoyl)-4-hydroxy-N—((S)-1-(4-(4-methylthiazol-5-yl)phenyl)ethyl)pyrrolidine-2-carboxamide (degrader #34): ¹H NMR (600 MHz, CDCl₃) δ 8.67 (s, 1H), 8.32-8.28 (m, 1H), 8.10 (dd, J=9.0, 4.4 Hz, 1H), 7.81-7.74 (m, 2H), 7.43-7.34 (m, 6H), 7.33-7.24 (m, 5H), 7.03 (d, J=8.5 Hz, 1H), 6.96 (d, J=8.4 Hz, 2H), 6.73 (d, J=7.4 Hz, 2H), 6.61 (d, J=9.4 Hz, 1H), 6.32 (d, J=8.2 Hz, 1H), 6.22 (s, 1H), 5.09 (dt, J=14.1, 7.8 Hz, 1H), 4.77-4.68 (m, 2H), 4.51 (s, 1H), 4.16 (d, J=9.8 Hz, 1H), 3.94-3.87 (m, 1H), 3.64 (t, J=8.2 Hz, 4H), 3.59 (dd, J=11.5, 2.8 Hz, 1H), 3.30-3.13 (m, 5H), 3.12-3.08 (m, 1H), 3.01 (dd, J=13.9, 7.2 Hz, 1H), 2.85-2.67 (m, 1H), 2.58-2.46 (m, 6H), 2.46-2.26 (m, 9H), 2.25-2.03 (m, 7H), 1.68-1.57 (m, 3H), 1.51-1.46 (m, 5H), 1.34-1.19 (m, 19H), 1.05 (d, J=3.6 Hz, 9H).

(2S,4R)-1-((2S)-2-(11-(4′-chloro-4-methyl-6-((4-(4-(((4-(((R)-4-morpholino-1-(phenylthio)butan-2-yl)amino)-3 ((trifluoromethyl)sulfonyl)phenyl)sulfonyl)carbamoyl)phenyl)piperazin-1-yl)methyl)-2,3,4,5-tetrahydro-[1,1′-biphenyl]-4-carboxamido)undecanamido)-3,3-dimethylbutanoyl)-4-hydroxy-N—((S)-1-(4-(4-methylthiazol-5-yl)phenyl)ethyl)pyrrolidine-2-carboxamide (degrader #35): ¹H NMR (600 MHz, CDCl₃) δ 8.67 (s, 1H), 8.30 (s, 1H), 8.11-8.07 (m, 1H), 7.77 (dd, J=11.8, 9.0 Hz, 2H), 7.38 (dt, J=17.1, 6.5 Hz, 6H), 7.27 (d, J=17.7 Hz, 5H), 7.03 (d, J=8.4 Hz, 1H), 6.96 (d, J=8.1 Hz, 2H), 6.72 (d, J=8.8 Hz, 2H), 6.61 (d, J=9.2 Hz, 1H), 6.35 (dd, J=28.5, 8.7 Hz, 1H), 6.18 (s, 1H), 5.11-5.05 (m, 1H), 4.71 (dd, J=7.8, 4.9 Hz, 2H), 4.51 (s, 1H), 4.16 (d, J=11.5 Hz, 1H), 3.90 (s, 1H), 3.66 (h, J=9.8, 9.2 Hz, 4H), 3.62-3.56 (m, 1H), 3.33-3.14 (m, 7H), 3.10 (dd, J=13.7, 4.8 Hz, 1H), 3.01 (dd, J=13.7, 7.2 Hz, 1H), 2.91-2.65 (m, 1H), 2.52 (s, 15H), 2.23-2.05 (m, 7H), 1.70-1.59 (m, 3H), 1.52-1.43 (m, 7H), 1.26 (d, J=4.5 Hz, 17H), 1.05 (d, J=2.8 Hz, 9H).

### Example 32: Preparation of Degraders #36-38

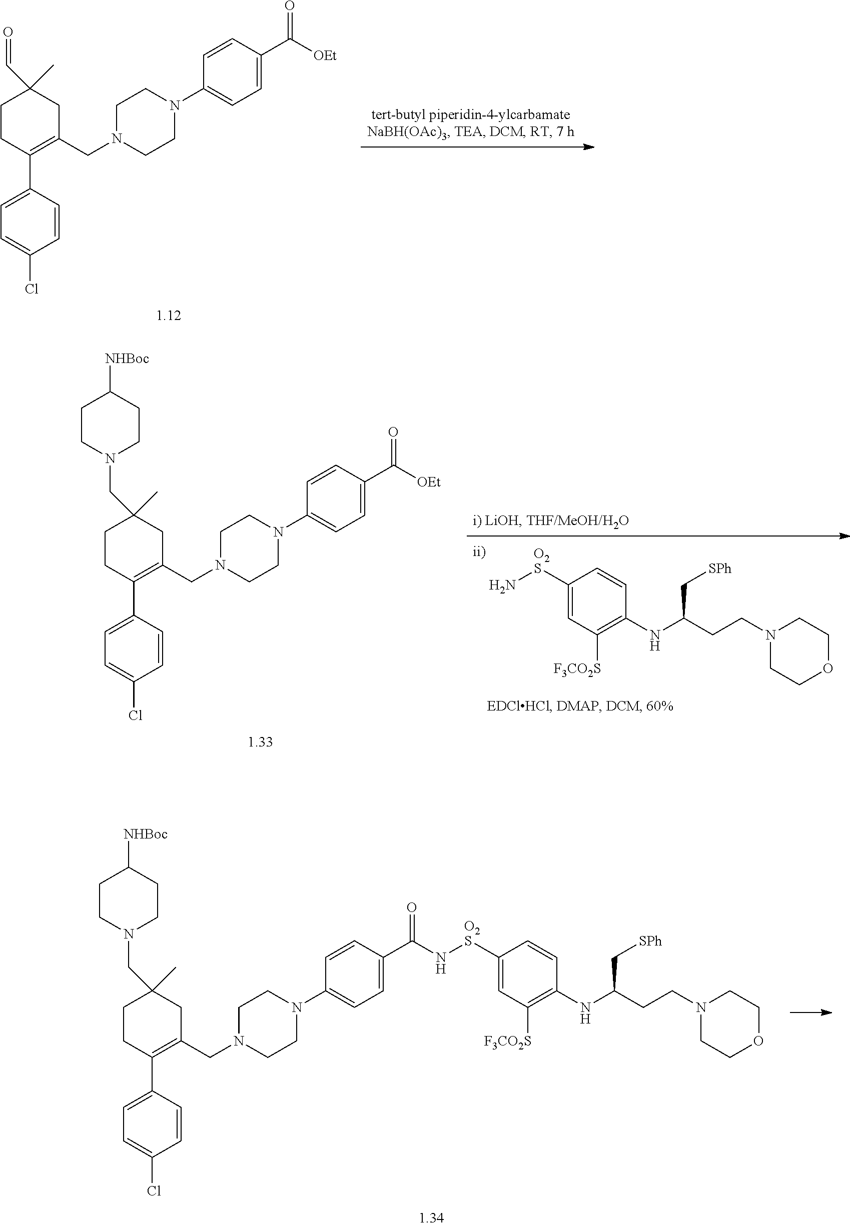

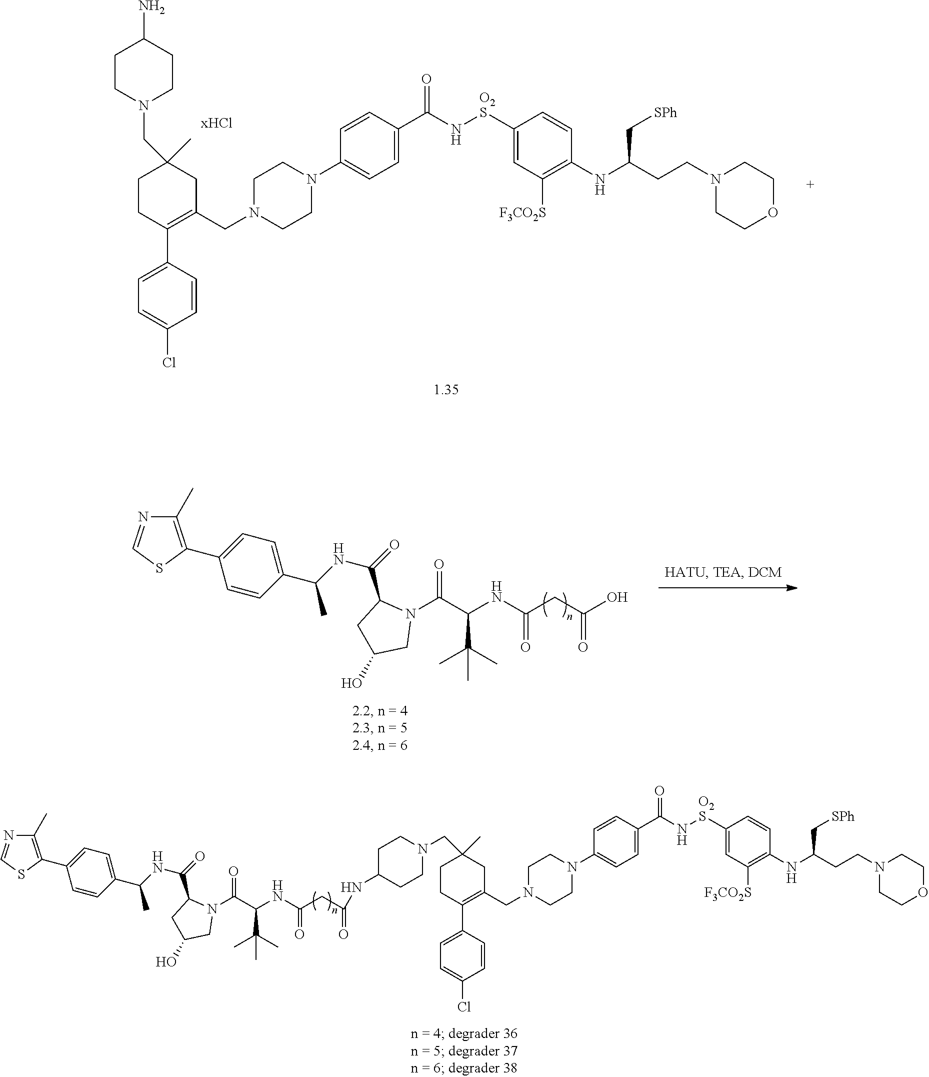

Preparation of ethyl 4-(4-((4-((4-((tert-butoxycarbonyl)amino)piperidin-1-yl)methyl)-4′-chloro-4-methyl-3,4,5,6-tetrahydro-[1,1′-biphenyl]-2-yl)methyl)piperazin-1-yl)benzoate (1.33): To a stirring solution of aldehyde 1.12 (1.0 equiv.) in DCM was added tert-butyl piperidin-4-ylcarbamate (1.5 equiv.), NaBH(OAc)₃ (7.0 equiv.) and TEA (10 equiv.). The resulting mixture was stirred at room temperature for 7 h. After the completion of the reaction, the reaction mixture was diluted with DCM and then washed with water followed by brine. The organic portion was dried over anhydrous MgSO₄, filtered, and concentrated under reduced pressure. The crude product was purified by silica gel column chromatography to afford the title compound. ¹H NMR (600 MHz, CDCl₃) δ 7.89 (d, J=9.0 Hz, 2H), 7.28 (s, 2H), 6.99 (d, J=8.4 Hz, 2H), 6.81 (d, J=9.1 Hz, 2H), 4.40 (s, 1H), 4.32 (q, J=7.1 Hz, 2H), 3.42 (s, 1H), 3.24 (t, J=5.0 Hz, 4H), 2.79 (s, 2H), 2.75 (d, J=11.5 Hz, 2H), 2.39-2.30 (m, 6H), 2.29-2.23 (m, 1H), 2.23-2.17 (m, 3H), 2.12 (d, J=17.3 Hz, 1H), 1.90 (d, J=17.0 Hz, 1H), 1.85 (d, J=8.1 Hz, 2H), 1.58 (d, J=6.7 Hz, 2H), 1.44 (s, 9H), 1.42 (d, J=4.5 Hz, 2H), 1.36 (t, J=7.1 Hz, 3H), 0.93 (s, 3H). ESI⁺, m/z [M+H]⁺=665.3.

Preparation of tert-butyl (1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-4-yl)carbamate (1.34): Compound 1.34 was prepared from compound 1.33 by following the same procedure as compound 1.9 was prepared from the compound 1.8. ¹H NMR (600 MHz, CDCl₃) δ 8.33 (s, 1H), 8.06 (d, J=9.1 Hz, 1H), 7.72 (d, J=7.5 Hz, 2H), 7.37 (d, J=7.4 Hz, 2H), 7.30 (t, J=7.5 Hz, 2H), 7.28-7.26 (m, 2H), 6.99 (d, J=7.3 Hz, 3H), 6.72 (d, J=7.6 Hz, 2H), 6.57 (d, J=9.0 Hz, 1H), 3.92-3.83 (m, 1H), 3.66 (p, J=7.2, 6.2 Hz, 4H), 3.47 (dd, J=12.5, 5.6 Hz, 1H), 3.24 (s, 4H), 3.10 (dd, J=13.8, 4.9 Hz, 1H), 3.00 (dd, J=13.9, 7.3 Hz, 1H), 2.95-2.85 (m, 3H), 2.52-2.40 (m, 8H), 2.40-2.29 (m, 8H), 2.29-2.16 (m, 5H), 2.16-2.08 (m, 2H), 2.05-1.98 (m, 1H), 1.85 (s, 2H), 1.67 (tt, J=14.2, 5.7 Hz, 2H), 1.62-1.49 (m, 1H), 1.43 (s, 9H), 0.96 (s, 3H).

Preparation of 4-(4-((4-((4-aminopiperidin-1-yl)methyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (1.35): Compound 1.35 was prepared from compound 1.34 by following the same procedure as compound 1.10 was prepared from compound 1.9. ESI⁺, m/z [M+H]⁺=1071.7.

General procedure for the preparation of degraders #36-38: Degraders #36-38 were prepared by following the same procedure as that of degrader 1 with amine 1.35 in place of amine 1.10.

N1-(1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-4-yl)-N6-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)adipamide (degrader #36): ¹H NMR (600 MHz, CDCl₃) δ 8.66 (s, 1H), 8.30 (s, 1H), 8.00 (d, J=8.5 Hz, 1H), 7.83-7.74 (m, 2H), 7.37 (d, J=7.3 Hz, 6H), 7.29 (t, J=7.5 Hz, 4H), 7.26-7.22 (m, 1H), 7.07-7.00 (m, 2H), 6.89 (d, J=7.0 Hz, 1H), 6.72-6.60 (m, 2H), 6.58 (d, J=8.3 Hz, 1H), 5.10 (q, J=7.1 Hz, 1H), 4.71 (s, 1H), 4.62 (t, J=7.7 Hz, 1H), 4.48 (s, 1H), 4.03 (t, J=8.8 Hz, 1H), 3.87 (s, 2H), 3.68-3.55 (m, 6H), 3.40-3.16 (m, 5H), 3.09 (d, J=4.7 Hz, 1H), 3.01-2.97 (m, 1H), 2.89-2.55 (m, 7H), 2.50 (s, 3H), 2.47-2.26 (m, 12H), 2.26-2.07 (m, 8H), 2.03-1.79 (m, 3H), 1.66 (dq, J=14.1, 5.8 Hz, 1H), 1.46 (t, J=6.6 Hz, 4H), 1.25 (s, 7H), 1.08 (s, 3H), 1.03 (s, 9H), 0.89-0.81 (m, 2H).

N1-(1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-4-yl)-N7-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)heptanediamide (degrader #37): ¹H NMR (600 MHz, CDCl₃) δ 8.67 (s, 1H), 8.31 (s, 1H), 8.04 (d, J=8.0 Hz, 1H), 7.76 (d, J=7.2 Hz, 2H), 7.41-7.32 (m, 7H), 7.32-7.26 (m, 5H), 7.00 (d, J=7.6 Hz, 2H), 6.97-6.91 (m, 1H), 6.71 (d, J=5.2 Hz, 1H), 6.57 (d, J=9.3 Hz, 1H), 6.38 (d, J=7.4 Hz, 1H), 5.08 (q, J=6.9 Hz, 1H), 4.71 (t, J=7.8 Hz, 1H), 4.61 (t, J=8.8 Hz, 1H), 4.49 (s, 1H), 4.07 (dd, J=10.6, 5.0 Hz, 1H), 3.89-3.75 (m, 2H), 3.65 (q, J=5.9, 5.5 Hz, 4H), 3.61-3.56 (m, 1H), 3.24 (dd, J=18.6, 7.0 Hz, 3H), 3.09 (dd, J=14.5, 4.8 Hz, 1H), 2.99 (dd, J=13.8, 7.3 Hz, 1H), 2.51 (s, 4H), 2.48-2.24 (m, 11H), 2.24-1.82 (m, 16H), 1.73-1.52 (m, 6H), 1.51-1.40 (m, 3H), 1.26 (d, J=9.2 Hz, 9H), 1.03 (s, 12H), 0.88 (t, J=6.9 Hz, 2H).

N1-(1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-4-yl)-N8-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)octanediamide (degrader #38): ¹H NMR (600 MHz, CDCl₃) δ 8.67 (s, 1H), 8.31 (s, 1H), 8.04 (d, J=9.0 Hz, 1H), 7.77 (d, J=8.2 Hz, 2H), 7.41-7.34 (m, 7H), 7.31-7.27 (m, 4H), 7.00 (d, J=7.8 Hz, 2H), 6.95-6.91 (m, 1H), 6.71-6.67 (m, 1H), 6.57 (d, J=9.4 Hz, 1H), 6.36-6.31 (m, 1H), 5.08 (td, J=7.3, 3.8 Hz, 1H), 4.69 (t, J=8.0 Hz, 1H), 4.59 (dd, J=8.7, 5.8 Hz, 1H), 4.48 (s, 1H), 4.08 (d, J=10.6 Hz, 1H), 3.91-3.77 (m, 2H), 3.68-3.61 (m, 4H), 3.57 (dt, J=11.0, 3.6 Hz, 1H), 3.33-3.16 (m, 5H), 3.09 (dd, J=13.8, 4.8 Hz, 1H), 2.99 (dd, J=13.8, 7.3 Hz, 1H), 2.96-2.85 (m, 2H), 2.51 (s, 3H), 2.45-2.26 (m, 15H), 2.24-2.03 (m, 9H), 1.90-1.79 (m, 2H), 1.66 (dd, J=14.2, 8.2 Hz, 2H), 1.56 (s, 4H), 1.49-1.40 (m, 4H), 1.25 (s, 9H), 1.03 (s, 9H), 1.01 (s, 3H), 0.96-0.80 (m, 2H).

### Example 33: Preparation of Degraders #39 and #40

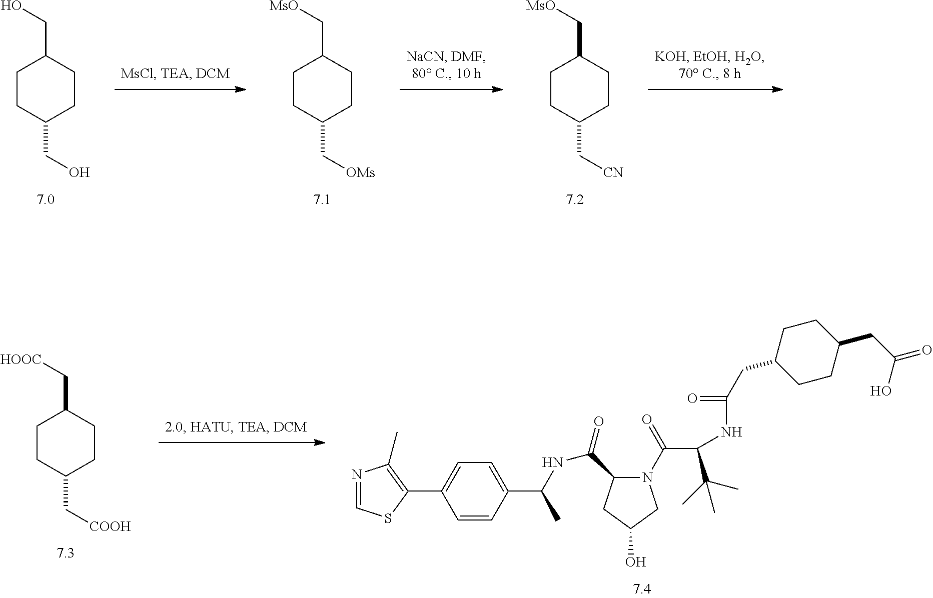

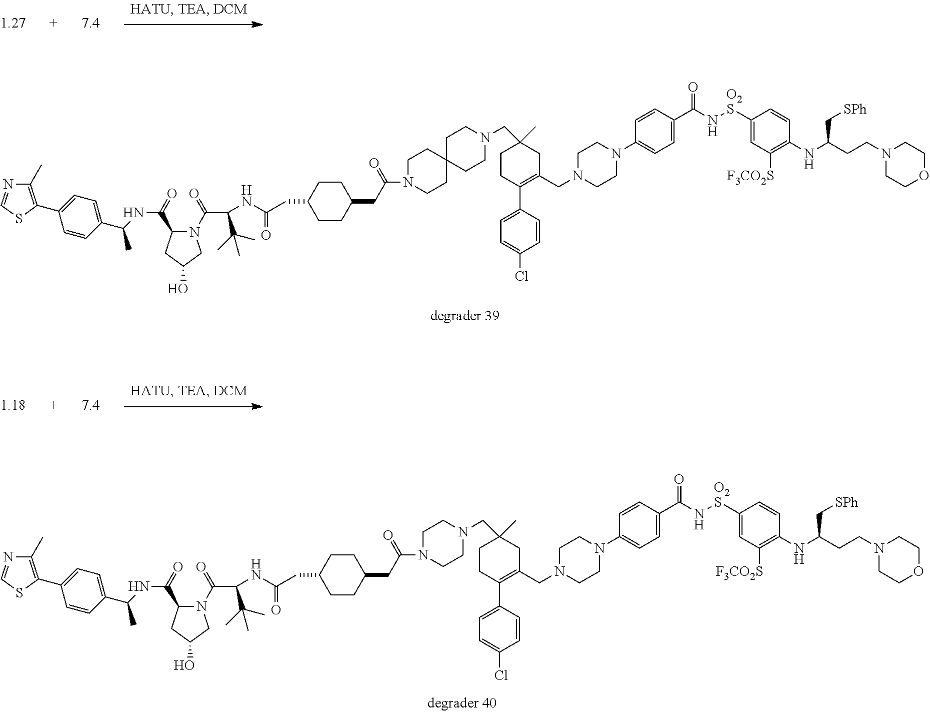

Preparation of (1,4-trans-cyclohexanediyl)bis(methylene) dimethanesulfonate (7.1): To a stirring solution of diol 7.0 (1.0 equiv.) and triethyl amine (5.0 equiv.) in DCM was added methanesulfonyl chloride (4.0 equiv.) dropwise at 0° C. The reaction was stirred for 7 h at room temperature and then diluted with DCM. The organic portion was washed with water and brine, dried over anhydrous Na₂SO₄, filtered, and concentrated under reduced pressure. The crude material was purified by flash chromatography. ¹H NMR (600 MHz, CDCl₃) δ 4.04 (d, J=6.3 Hz, 4H), 3.00 (s, 6H), 1.89 (d, J=7.0 Hz, 4H), 1.76-1.69 (m, 2H), 1.08 (td, J=9.3, 3.3 Hz, 4H).

Preparation of 2,2′-(1,4-trans-cyclohexanediyl)diacetonitrile (7.2): To a stirring solution of diol 7.1 (1.0 equiv.) in DMF was added NaCN (4.0 equiv.) and the reaction was stirred at 70° C. for 10 h. Once the reaction was complete (monitored by TLC), the reaction mixture was diluted with ethyl acetate. The organic portion was washed with water and brine, dried over anhydrous Na₂SO₄, filtered, and concentrated under reduced pressure. The crude material was purified by flash chromatography. ¹H NMR (600 MHz, CDCl₃) δ 2.28 (d, J=6.5 Hz, 4H), 1.93 (d, J=7.2 Hz, 4H), 1.68-1.63 (m, 2H), 1.22-1.12 (m, 4H).

2,2′-(1,4-trans-cyclohexanediyl)diacetic acid (7.3): To a stirring solution of compound 7.2 (1.0 equiv.) in ethanol was added KOH (5.0 equiv.) solution and the reaction was stirred 8 h at 70° C. Once the reaction was complete, the pH of the reaction was adjusted to pH 7 with 3N HCl solution. The ethanol from the reaction mixture was then evaporated and the remaining solid was collected by filtration. The solid was washed with cold water and diethyl ether. The solid then dried in reduced pressure to the title compound 7.3. ¹H NMR (600 MHz, CD₃OD) 2.09 (d, J=7.1 Hz, 4H), 1.73 (d, J=7.0 Hz, 4H), 1.66-1.57 (m, 2H), 0.97 (dd, J=11.2, 9.3 Hz, 4H).

Preparation of 2-((1,4-trans)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)acetic acid (7.4): A mixture of amine 2.0 (1.0 equiv.), acid 7.3 (1.1 equiv.), HATU (1.2 equiv.) and TEA (5.0 equiv.) was taken in DCM and the reaction mixture was stirred at room temperature for 4 h. After completion of the reaction, DCM was evaporated and the crude product was purified by column chromatography. ¹H NMR (600 MHz, CDCl₃) δ 8.67 (s, 1H), 7.40 (d, J=8.2 Hz, 2H), 7.36 (d, J=8.3 Hz, 3H), 6.51 (d, J=8.5 Hz, 1H), 5.08 (p, J=7.0 Hz, 1H), 4.70 (t, J=7.9 Hz, 1H), 4.59 (d, J=8.9 Hz, 1H), 4.50 (s, 1H), 4.15 (d, J=11.5 Hz, 1H), 3.58 (dd, J=11.4, 3.5 Hz, 1H), 2.52 (s, 3H), 2.51-2.47 (m, 2H), 2.14 (d, J=7.0 Hz, 2H), 2.10 (d, J=6.8 Hz, 2H), 2.08-2.04 (m, 1H), 1.82-1.75 (m, 2H), 1.75-1.70 (m, 2H), 1.69 (d, J=7.2 Hz, 2H), 1.47 (d, J=6.9 Hz, 3H), 1.03 (s, 9H), 1.01 (d, J=10.3 Hz, 3H).

General procedure for the synthesis of degraders #39 and #40: To a stirring solution of amines 1.28 or 1.18 (12 mg, 0.011 mmol) and acid 7.4 (7 mg, 0.012 mmol) in DCM (1 mL) was added TEA (0.01 mL, 0.066 mmol) at room temperature. To the mixture HATU (5 mg, 0.012 mmol) was added and the reaction were stirred for 8 h at the same temperature. Upon completion of the reaction, solvent was removed under reduced pressure and the crude product was purified by flash column chromatography (DCM/MeOH=96:4). The product from column was mixed with 15 mL DCM and washed with saturated aqueous NH₄Cl. The organic portion was dried over Na₂SO₄, filtered, and DCM was evaporated under reduced pressure to afford the title compound.

(2S,4R)-1-((2S)-2-(2-((1,4-trans)-4-(2-(9-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-3,9-diazaspiro[5.5]undecan-3-yl)-2-oxoethyl)cyclohexyl)acetamido)-3,3-dimethylbutanoyl)-4-hydroxy-N—((S)-1-(4-(4-methylthiazol-5-yl)phenyl)ethyl)pyrrolidine-2-carboxamide (degrader #39): ¹H NMR (600 MHz, CDCl₃) δ 8.67 (s, 1H), 8.33 (d, J=2.0 Hz, 1H), 8.05 (d, J=10.3 Hz, 1H), 7.76 (d, J=8.6 Hz, 2H), 7.46-7.33 (m, 8H), 7.31-7.23 (m, 3H), 6.98 (d, J=8.2 Hz, 2H), 6.94 (s, 1H), 6.73-6.64 (m, 2H), 6.57 (d, J=9.4 Hz, 1H), 6.19-6.14 (m, 1H), 5.07 (p, J=7.0 Hz, 1H), 4.72 (td, J=7.9, 3.4 Hz, 1H), 4.55 (d, J=8.1 Hz, 1H), 4.50 (s, 1H), 4.15-4.10 (m, 1H), 3.87 (d, J=10.3 Hz, 1H), 3.68-3.62 (m, 4H), 3.58 (dd, J=11.4, 3.5 Hz, 1H), 3.54-3.47 (m, 2H), 3.34 (s, 2H), 3.22 (s, 3H), 3.10 (dd, J=13.8, 5.0 Hz, 1H), 2.99 (dd, J=13.8, 7.4 Hz, 1H), 2.95-2.86 (m, 1H), 2.77-2.63 (m, 2H), 2.52 (s, 5H), 2.50-2.24 (m, 14H), 2.22-1.98 (m, 9H), 1.78-1.66 (m, 8H), 1.66-1.53 (m, 3H), 1.46 (dd, J=7.0, 1.5 Hz, 3H), 1.41 (d, J=22.5 Hz, 4H), 1.04 (s, 9H), 1.01 (s, 3H), 1.00-0.92 (m, 4H), 0.91-0.79 (m, 4H).

(2S,4R)-1-((2S)-2-(2-((1,4-trans)-4-(2-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-2-oxoethyl)cyclohexyl)acetamido)-3,3-dimethylbutanoyl)-4-hydroxy-N—((S)-1-(4-(4-methylthiazol-5-yl)phenyl)ethyl)pyrrolidine-2-carboxamide (degrader #40): ¹H NMR (600 MHz, CDCl₃) δ 8.67 (s, 1H), 8.30 (dd, J=17.6, 2.1 Hz, 1H), 8.10 (td, J=9.3, 2.0 Hz, 1H), 7.80 (d, J=9.0 Hz, 1H), 7.74 (d, J=8.9 Hz, 1H), 7.41-7.27 (m, 11H), 7.04 (d, J=8.6 Hz, 1H), 6.99 (d, J=8.3 Hz, 2H), 6.75 (d, J=7.6 Hz, 2H), 6.61 (dd, J=9.4, 3.5 Hz, 1H), 6.29 (dd, J=43.5, 8.8 Hz, 1H), 5.12-5.06 (m, 1H), 4.74-4.64 (m, 2H), 4.51 (s, 1H), 4.18-4.10 (m, 1H), 3.93-3.86 (m, 1H), 3.74-3.63 (m, 5H), 3.62-3.33 (m, 4H), 3.26 (d, J=32.2 Hz, 4H), 3.18-3.06 (m, 2H), 3.02 (ddd, J=13.9, 7.2, 1.9 Hz, 1H), 2.86 (s, 1H), 2.65-2.57 (m, 1H), 2.52 (d, J=3.3 Hz, 7H), 2.43 (s, 3H), 2.41-2.28 (m, 8H), 2.26 (d, J=8.1 Hz, 3H), 2.23-2.17 (m, 2H), 2.17-1.99 (m, 6H), 1.85 (dd, J=38.2, 18.4 Hz, 1H), 1.77-1.55 (m, 8H), 1.47 (d, J=6.9 Hz, 3H), 1.05 (d, J=2.5 Hz, 9H), 1.00 (d, J=12.8 Hz, 3H), 0.94 (d, J=11.7 Hz, 3H), 0.89 (dt, J=9.1, 6.7 Hz, 2H).

### Example 34: Preparation of Degraders #41 and #42

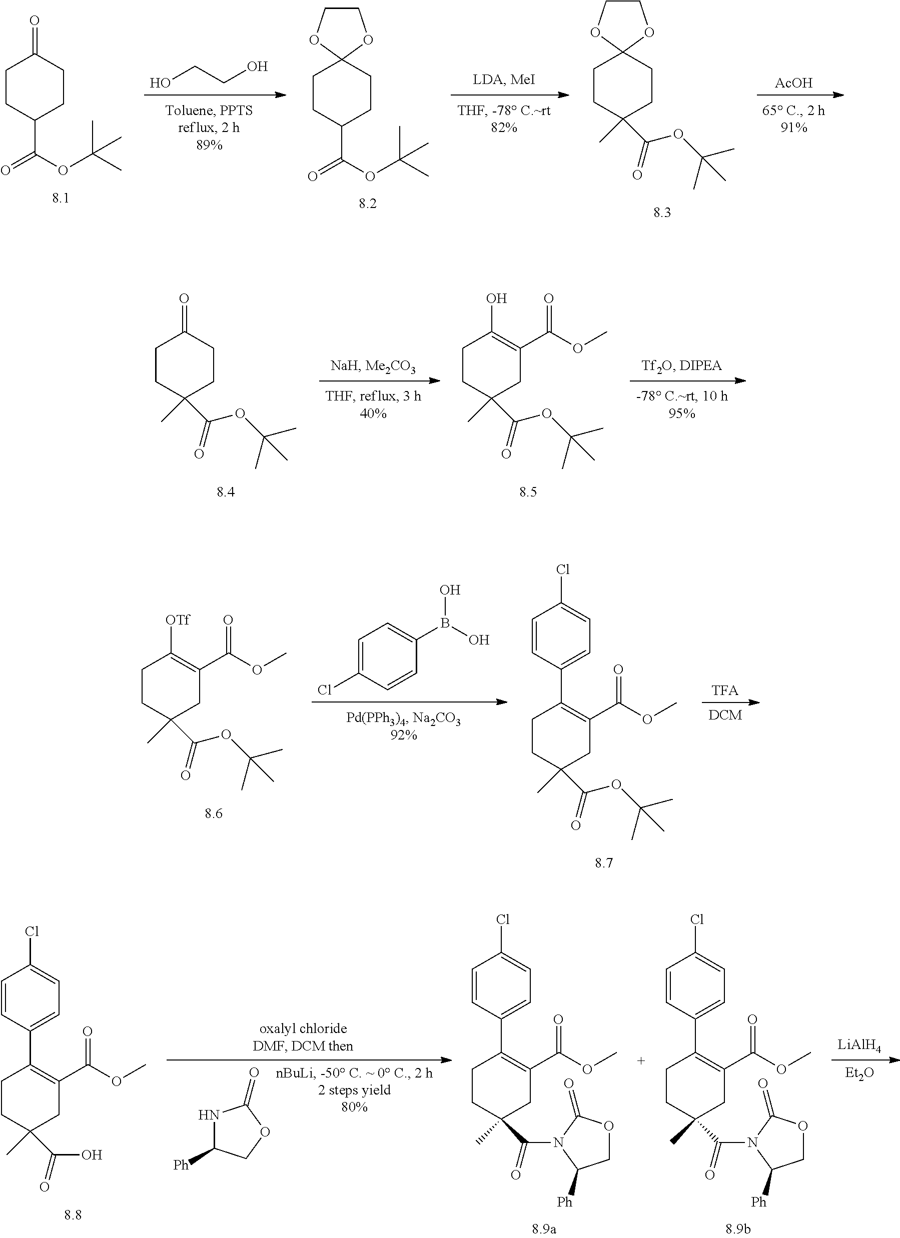

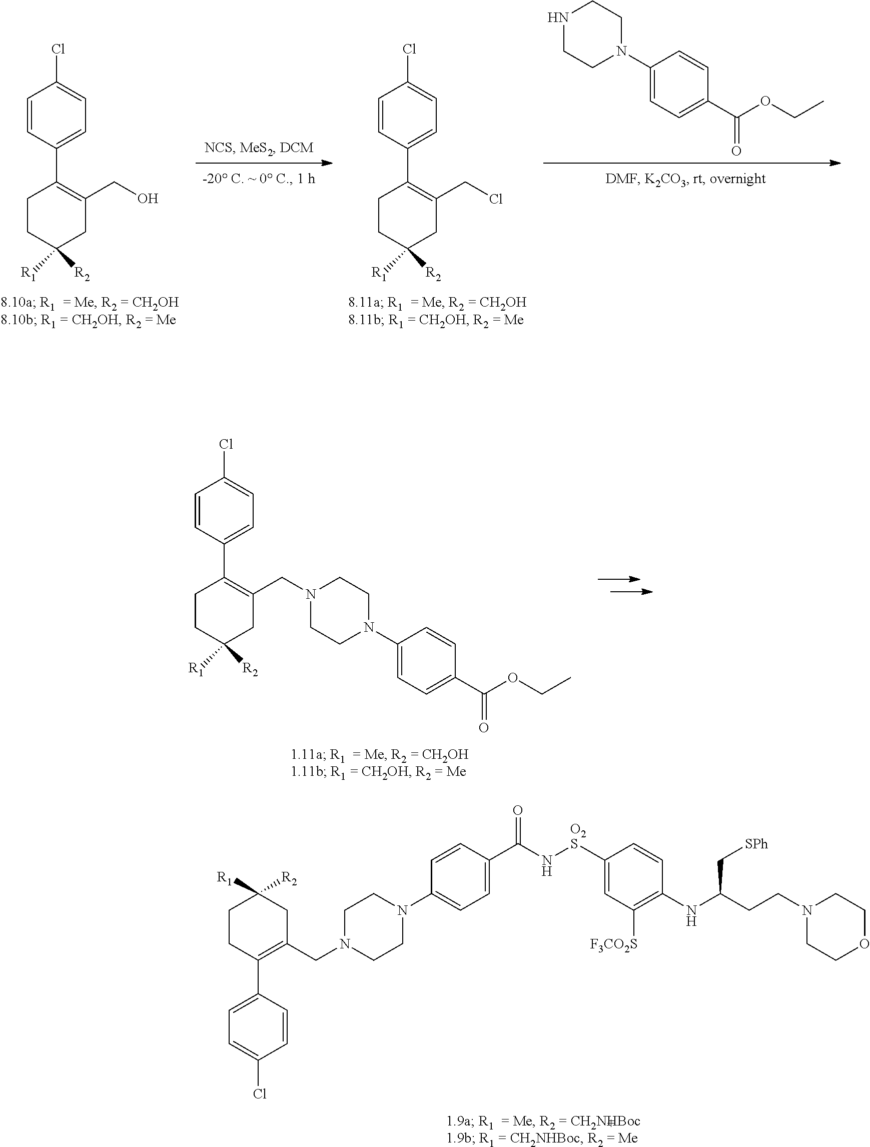

Preparation of tert-butyl 1,4-dioxaspiro[4.5]decane-8-carboxylate (8.2): To a stirring solution of compound 8.1 (1.0 equiv.) in toluene was added ethylene glycol (1.5 equiv.) and PPTS (5 mol %). The mixture was refluxed in a Dean-Stark apparatus for 2 h. Once the reaction was complete, temperature of the reaction was allowed to cool to room temperature and TEA was added to the mixture. The reaction mixture was diluted with ethyl acetate. The organic portion was washed with saturated aqueous NaHCO₃ solution and brine, dried over anhydrous Na₂SO₄, filtered, and concentrated under reduced pressure. The crude material was purified by flash chromatography to afford the title compound. ¹H NMR (600 MHz, CDCl₃) δ 3.94 (s, 4H), 2.27-2.19 (m, 1H), 1.94-1.85 (m, 2H), 1.81-1.70 (m, 4H), 1.57-1.50 (m, 2H), 1.43 (s, 9H).

Preparation of tert-butyl 8-methyl-1,4-dioxaspiro[4.5]decane-8-carboxylate (8.3): To a stirring solution of compound 8.2 (1.0 equiv.) in THF was added 1N LDA solution (1.5 equiv.) dropwise at −78° C. The temperature of the reaction was allowed to warm to room temperature slowly and the reaction was stirred at this temperature for 4 h. Once all the starting material was consumed, the reaction was quenched with saturated aqueous NH₄Cl solution. THF was removed under reduced pressure and the crude product was diluted with ethyl acetate. The organic portion was washed with water and brine, dried over anhydrous Na₂SO₄, filtered, and concentrated under reduced pressure. The crude material was purified by flash chromatography to afford the title compound. ¹H NMR (600 MHz, CDCl₃) δ 3.93 (s, 4H), 2.11-2.05 (m, 2H), 1.68-1.58 (m, 4H), 1.49-1.41 (m, 11H), 1.15 (s, 3H).

Preparation of tert-butyl 1-methyl-4-oxocyclohexane-1-carboxylate (8.4): Compound 8.3 was mixed with acetic acid and stirred for 2 h at 65° C. After completion of the reaction, acetic acid was removed under reduced pressure and the residue was diluted with ethyl acetate. The mixture was washed with water and brine, dried over anhydrous Na₂SO₄, filtered, and concentrated under reduced pressure. The crude material was purified by flash chromatography to afford the title compound. ¹H NMR (600 MHz, CDCl₃) δ 2.48-2.39 (m, 2H), 2.39-2.26 (m, 4H), 1.67-1.56 (m, 2H), 1.48 (s, 9H), 1.26 (s, 3H).

Preparation of 1-(tert-butyl) 3-methyl 4-hydroxy-1-methylcyclohex-3-ene-1,3-dicarboxylate (8.5): Compound 8.5 was synthesized from compound 8.4 by following the same procedure as that of compound 1.2 was prepared from compound 1.1. ¹H NMR (600 MHz, CDCl₃) δ 12.12 (s, 1H), 3.76 (s, 3H), 2.76 (dq, J=15.8, 1.3 Hz, 1H), 2.38 (dddt, J=20.6, 8.3, 6.5, 1.6 Hz, 1H), 2.29 (tt, 1H), 2.07-1.99 (m, 2H), 1.61-1.56 (m, 1H), 1.42 (s, 9H), 1.21 (s, 3H).

Preparation of 1-(tert-butyl) 3-methyl 1-methyl-4-(((trifluoromethyl)sulfonyl)oxy)cyclohex-3-ene-1,3-dicarboxylate (8.6): Compound 8.6 was synthesized from compound 8.5 by following the same procedure as that of compound 1.3 was prepared from compound 1.2. ¹H NMR (600 MHz, CDCl₃) δ 3.83 (s, 3H), 3.04 (dd, J=17.6, 2.0 Hz, 1H), 2.62-2.51 (m, 1H), 2.47-2.37 (m, 1H), 2.30 (tt, J=17.6, 3.4, 2.4 Hz, 1H), 2.19-2.12 (m, 1H), 1.67 (ddd, J=13.3, 8.7, 6.3 Hz, 1H), 1.46 (s, 9H), 1.27 (s, 3H).

Preparation of 4-(tert-butyl) 2-methyl 4′-chloro-4-methyl-3,4,5,6-tetrahydro-[1,1′-biphenyl]-2,4-dicarboxylate (8.7): Compound 8.7 was synthesized from compound 8.6 following the same procedure as that of compound 1.4 was prepared from compound 1.3. ¹H NMR (600 MHz, CDCl₃) δ 7.29-7.26 (m, 2H), 7.04-6.99 (m, 2H), 3.47 (s, 3H), 2.93 (dq, J=17.7, 2.0 Hz, 1H), 2.51-2.41 (m, 1H), 2.40-2.32 (m, 1H), 2.22 (dt, J=17.5, 2.7 Hz, 1H), 2.12-2.05 (m, 1H), 1.65-1.58 (m, 1H), 1.45 (s, 9H), 1.26 (s, 3H).

Preparation of 4′-chloro-6-(methoxycarbonyl)-4-methyl-2,3,4,5-tetrahydro-[1,1′-biphenyl]-4-carboxylic acid (8.8): To a stirring solution of compound 8.7 in DCM was added TFA (10 equiv.) and the reaction was stirred for 4 h at room temperature. After completion of the reaction, the volatiles were removed in reduced pressure to afford the title compound, which was used in the next step without further purification.

Preparation of methyl (S)-4′-chloro-4-methyl-4-((R)-2-oxo-4-phenyloxazolidine-3-carbonyl)-3,4,5,6-tetrahydro-[1,1′-biphenyl]-2-carboxylate (8.9a) and methyl (R)-4′-chloro-4-methyl-4-((R)-2-oxo-4-phenyloxazolidine-3-carbonyl)-3,4,5,6-tetrahydro-[1,1′-biphenyl]-2-carboxylate (8.9b): To a stirring solution of acid 8.8 (1.0 equiv.) in DCM was added oxalyl chloride (1.5 equiv.) and the mixture was stirred at room temperature for 4 h. Once the reaction was completed, the volatiles were removed under reduced pressure and the crude acid chloride was used in the next step without further purification.

To a stirring solution of (R)-4-phenyloxazolidin-2-one (1.5 equiv.) in THF was added 1M nBuLi solution (1.5 equiv.) dropwise at −78° C. The reaction was stirred at the same temperature for 30 min. A solution of acid chloride in THF was added dropwise at the same temperature. Once the acid chloride was completely consumed, the reaction was quenched with saturated aqueous NH₄Cl solution. THF was removed under reduced pressure and the crude product was diluted with ethyl acetate. The organic portion was washed with water and brine, dried over anhydrous Na₂SO₄, filtered, and concentrated under reduced pressure. The crude material was purified by flash chromatography to afford the title compound 8.9a and 8.9b in equal amount.

8.9a: ¹H NMR (600 MHz, CDCl₃) δ 7.36-7.31 (m, 3H), 7.31-7.27 (m, 2H), 7.22-7.17 (m, 2H), 6.77-6.71 (m, 2H), 5.54 (dd, J=8.9, 5.5 Hz, 1H), 4.69 (t, J=8.9 Hz, 1H), 4.22 (dd, J=9.0, 5.5 Hz, 1H), 3.50 (s, 3H), 3.43-3.35 (m, 1H), 2.48-2.39 (m, 1H), 2.35-2.23 (m, 3H), 1.82-1.74 (m, 1H), 1.53 (s, 3H).

8.9b: ¹H NMR (600 MHz, CDCl₃) δ 7.40-7.32 (m, 5H), 7.24-7.19 (m, 2H), 6.77-6.72 (m, 2H), 5.52 (dd, J=8.6, 4.0 Hz, 1H), 4.75 (t, J=8.8 Hz, 1H), 4.32 (dd, J=8.9, 3.9 Hz, 1H), 3.47 (s, 3H), 3.10 (dd, J=17.8, 2.0 Hz, 1H), 2.64-2.56 (m, 1H), 2.46-2.38 (m, 1H), 2.37-2.27 (m, 1H), 2.18-2.09 (m, 1H), 1.86-1.77 (m, 1H), 1.55 (s, 3H).

Preparation of (4′-chloro-4-methyl-3,4,5,6-tetrahydro-[1,1′-biphenyl]-2,4-diyl)dimethanol (8.10a and 8.10b): To a stirring solution of compound 8.9a or 8.9b (1.0 equiv.) in diethyl ether was added LAH (2.0 equiv.) portionwise at 0° C. and the resulting mixture was stirred for 2 h. Upon completion, the reaction was quenched with 10% NaOH solution followed by adding anhydrous MgSO₄. The mixture was filtered and the filtrate was concentrated under reduced pressure to give the crude product which was purified by flash chromatography to afford the title compound 8.10a or 8.10b. ¹H NMR (600 MHz, CDCl₃) δ 7.31-7.27 (m, 2H), 7.10-7.06 (m, 2H), 3.97-3.89 (m, 2H), 3.47 (s, 2H), 2.37-2.26 (m, 2H), 2.22 (d, J=17.4, 2.5 Hz, 1H), 1.99 (d, J=17.4, 1.8 Hz, 1H), 1.66-1.59 (m, 1H), 1.53-1.45 (m, 1H), 1.01 (s, 3H).

Preparation of (4′-chloro-6-(chloromethyl)-4-methyl-2,3,4,5-tetrahydro-[1,1′-biphenyl]-4-yl)methanol (8.11a and 8.11b): To a stirring solution of diol 8.10a or 8.10b (1.0 equiv.) and NCS (1.2 equiv.) in DCM was added DMS (1.2 equiv.) at −20° C. The reaction was stirred for 1 h at 0° C. Upon completion, the mixture was diluted with DCM and washed with water and brinen. The organic portion was dried over anhydrous Na₂SO₄, filtered, and concentrated under reduced pressure. The crude material was purified by flash chromatography to afford the title compound 8.11a or 8.11b. ¹H NMR (600 MHz, DMSO-d6) δ 7.50-7.40 (m, 2H), 7.29-7.18 (m, 2H), 4.72-4.41 (br, 1H), 3.96 (s, 2H), 3.21 (dd, 2H), 2.33-2.21 (m, 2H), 2.13 (d, J=17.3, 2.6 Hz, 1H), 1.89 (d, J=17.3, 1.8 Hz, 1H), 1.60-1.50 (m, 1H), 1.39-1.32 (m, 1H), 0.89 (s, 3H).

Preparation of ethyl 4-(4-((4′-chloro-4-(hydroxymethyl)-4-methyl-3,4,5,6-tetrahydro-[1,1′-biphenyl]-2-yl)methyl)piperazin-1-yl)benzoate (1.11a and 1.11b): Compound 8.11a or 8.11b (1.0 equiv.) was dissolved in DMF followed by the addition of K₂CO₃ (1.5 equiv.) and ethyl 4-(piperazin-1-yl)benzoate (1.2 equiv.). The mixture was stirred at 75° C. for 24 h. Upon consumption, the mixture was allowed to cool to room temperature and diluted with EtOAc and successively washed with water (25 mL×3) and brine. The organic portion was dried over anhydrous Na₂SO₄, filtered, and concentrated under reduced pressure. The crude material was purified by flash chromatography (Hexanes/EtOAc=2:1) to afford the title compound 1.11a or 1.11b. ¹H NMR 1.11a and 1.11b are same as that of compound 1.11.

Preparation of tert-butyl (((S)-4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)carbamate (1.9a): Compound 1.9a was prepared from alcohol 1.11a by following the same synthetic protocol as compound 1.9 was prepared from alcohol 1.11. ¹H NMR (600 MHz, CDCl₃) δ 8.34 (s, 1H), 8.07 (d, 1H), 7.66 (br, 2H), 7.36 (d, J=7.5 Hz, 2H), 7.32-7.17 (m, 4H), 7.06-6.92 (m, 3H), 6.73 (br, 2H), 6.57 (br, 1H), 4.74 (br, 1H), 3.87 (br, 1H), 3.69-3.54 (m, 4H), 3.23 (br, 4H), 3.16-2.92 (m, 4H), 2.87-2.72 (m, 2H), 2.48-2.19 (m, 12H), 2.09 (d, J=17.0 Hz, 2H), 1.97 (d, J=17.3 Hz, 1H), 1.82-1.51 (m, 3H), 1.51-1.44 (m, 1H), 1.42 (s, 9H), 0.96 (s, 3H).

Preparation of tert-butyl (((R)-4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)carbamate (1.9b): Compound 1.9b was prepared from alcohol 1.11b by following the same synthetic protocol as compound 1.9 was prepared from alcohol 1.11. ¹H NMR (600 MHz, CDCl₃) δ 8.35 (d, J=2.2 Hz, 1H), 8.09 (dd, J=9.3, 2.3 Hz, 1H), 7.69-7.61 (m, 2H), 7.40-7.33 (m, 2H), 7.33-7.19 (m, 4H), 7.04 (d, J=8.5 Hz, 1H), 6.99 (d, J=8.3 Hz, 2H), 6.75 (d, J=8.7 Hz, 2H), 6.60 (d, J=9.3 Hz, 1H), 4.82 (br, 1H), 3.94-3.83 (m, 1H), 3.73-3.58 (m, 4H), 3.27 (t, J=5.2 Hz, 4H), 3.16-3.06 (m, 2H), 3.06-2.95 (m, 2H), 2.88 (s, 2H), 2.51-2.19 (m, 12H), 2.16-2.07 (m, 2H), 1.99 (d, J=17.4 Hz, 1H), 1.73-1.62 (m, 1H), 1.58-1.51 (m, 1H), 1.51-1.44 (m, 2H), 1.41 (s, 9H), 0.95 (s, 3H).

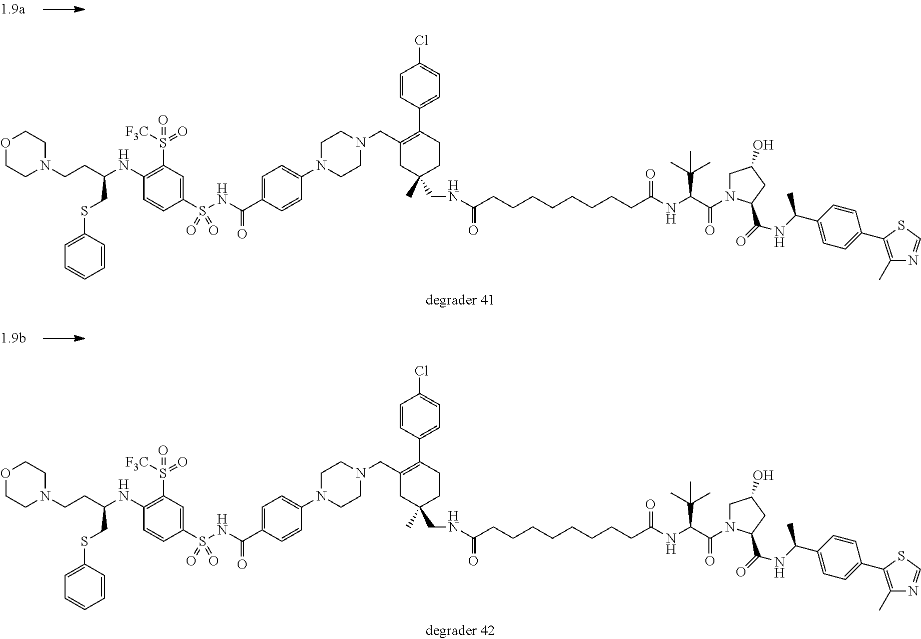 [SMILES: Cc1ncsc1-c1ccc([C@H](C)NC(=O)[C@@H]2C[C@@H](O)CN2C(=O)[C@@H](NC(=O)CCCCCCCCC(=O)NC[C@@]2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1 ; Cc1ncsc1-c1ccc([C@H](C)NC(=O)[C@@H]2C[C@@H](O)CN2C(=O)[C@@H](NC(=O)CCCCCCCCC(=O)NC[C@]2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

Preparation of N1-(((S)-4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N10-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)decanediamide (degrader #41): To a stirring solution of compound 1.9a (1.0 equiv.) in DCM was added HCl (10 equiv.) in dioxane. After completion of the reaction, the volatiles were removed under reduced pressure to afford a crude off-white power. The crude product was dissolved in DCM followed by the addition of acid 2.6 (1.1 equiv.), HATU (1.2 equiv.), TEA (10 equiv.) and the mixture was stirred for 8 h. Upon completion, solvent was removed under reduced pressure and the crude product was purified by flash column chromatography (DCM/MeOH/TEA=96:4:1). The product from the column was mixed with 15 mL DCM and washed with saturated aqueous NH₄Cl solution. The organic portion was dried over Na₂SO₄, filtered, and concentrated under reduced pressure to afford the title compound. ¹H NMR (600 MHz, CDCl₃) δ 8.67 (s, 1H), 8.28 (s, 1H), 8.02 (br, 1H), 7.82 (d, J=8.5 Hz, 2H), 7.42-7.33 (m, 7H), 7.33-7.17 (m, 5H), 7.02 (d, J=7.9 Hz, 2H), 6.93 (d, J=8.3 Hz, 1H), 6.74 (d, J=8.6 Hz, 2H), 6.58 (d, J=9.3 Hz, 1H), 6.32 (br, 1H), 5.13-5.01 (m, 1H), 4.72 (t, J=8.0 Hz, 1H), 4.62 (br, 1H), 4.51 (s, 1H), 4.16 (d, J=11.6 Hz, 1H), 3.87 (br, 1H), 3.72-3.53 (m, 5H), 3.35-3.13 (m, 5H), 3.09 (dd, J=13.7, 5.3 Hz, 1H), 3.00 (dd, J=13.7, 7.3 Hz, 1H), 2.55-2.49 (m, 4H), 2.49-2.23 (m, 15H), 2.23-2.14 (m, 2H), 2.14-2.04 (m, 6H), 1.77-1.61 (m, 1H), 1.61-1.50 (m, 5H), 1.47 (d, J=7.0 Hz, 3H), 1.44-1.31 (m, 2H), 1.30-1.18 (m, 2H), 1.18-1.08 (m, 5H), 1.05 (s, 9H), 0.99 (s, 3H).

N1-(((R)-4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N10-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)decanediamide (degrader #42): Degrader #42 was prepared from compound 1.9b by following the same synthetic procedure as described for the preparation of degrader 41. ¹H NMR (600 MHz, CDCl₃) δ 8.67 (s, 1H), 8.30 (d, J=2.3 Hz, 1H), 8.05 (d, 1H), 7.75 (d, 2H), 7.45 (br, 1H), 7.41-7.33 (m, 6H), 7.32-7.27 (m, 4H), 7.29-7.21 (m, 1H), 7.03-6.98 (m, 2H), 6.95 (d, J=8.5 Hz, 1H), 6.66 (d, J=8.5 Hz, 2H), 6.60 (d, J=9.3 Hz, 1H), 6.27 (d, J=8.3 Hz, 1H), 6.09 (br, 1H), 5.09 (p, J=7.1 Hz, 1H), 4.70 (t, J=8.1 Hz, 1H), 4.60 (br, 1H), 4.49 (br, 1H), 4.12 (d, J=11.4 Hz, 1H), 3.88 (br, 1H), 3.69-3.61 (m, 4H), 3.59 (d, J=10.7 Hz, 1H), 3.35-3.27 (m, 1H), 3.27-3.15 (m, 4H), 3.15-3.05 (m, 2H), 3.00 (dd, J=13.8, 7.2 Hz, 1H), 2.63-2.46 (m, 6H), 2.41 (br, 4H), 2.38-2.26 (m, 8H), 2.23 (d, J=17.5 Hz, 1H), 2.16 (t, J=7.6 Hz, 2H), 2.13-2.00 (m, 6H), 1.70-1.61 (m, 1H), 1.61-1.50 (m, 5H), 1.48 (d, J=7.0 Hz, 3H), 1.44-1.35 (m, 2H), 1.22-1.14 (m, 2H), 1.14-1.00 (m, 14H), 0.98 (s, 3H).

### Example 35: Preparation of Degraders #43 and #44

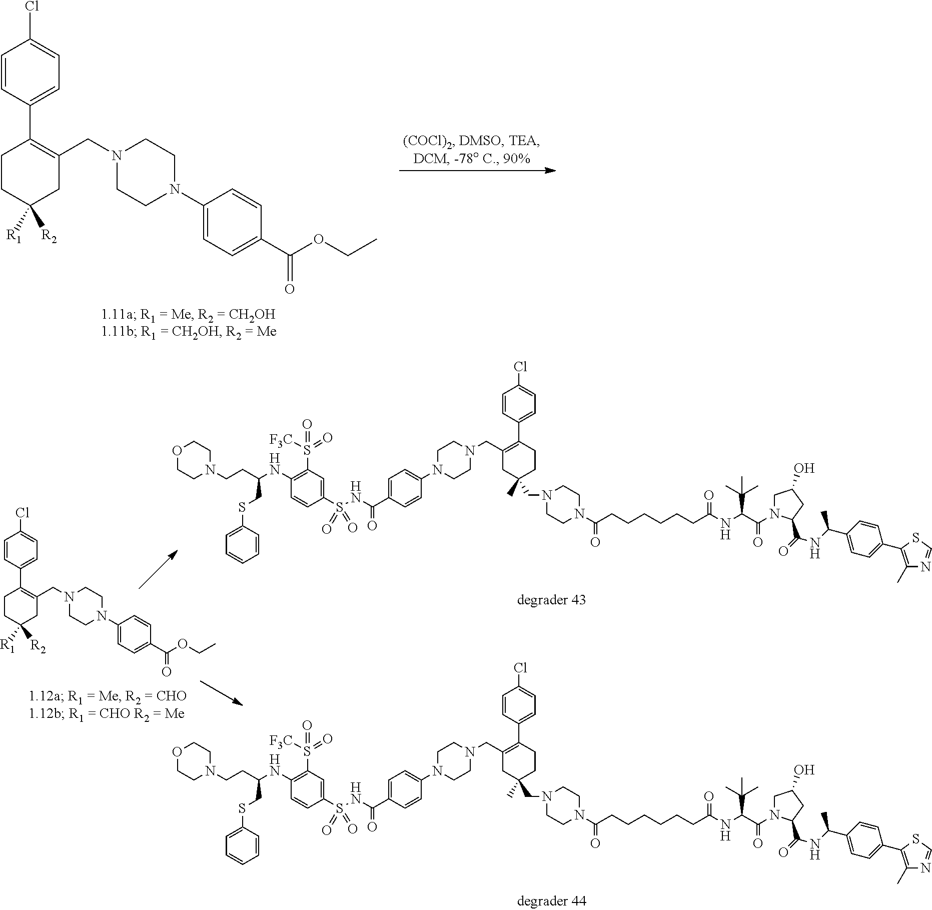

Preparation of ethyl 4-(4-((4′-chloro-4-formyl-4-methyl-3,4,5,6-tetrahydro-[1,1′-biphenyl]-2-yl)methyl)piperazin-1-yl)benzoate (1.12a and 1.12b): Alcohol 1.11a and 1.11b were converted to the aldehyde 1.12a and 1.12b, respectively, by following the same procedure as that of aldehyde 1.12 was prepared from alcohol 1.11. ¹H NMR of 1.12a and 1.12b are same as that of aldehyde 1.12.

Preparation of (2S,4R)-1-((S)-2-(8-(4-(((S)-4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #43): Degrader 43 was prepared from the aldehyde 1.12a by following the same protocol as degrader 17 was prepared from aldehyde 1.12. ¹H NMR (600 MHz, CDCl₃) δ 8.67 (s, 1H), 8.32 (d, J=2.2 Hz, 1H), 8.08 (d, J=9.1 Hz, 1H), 7.72 (d, J=8.5 Hz, 2H), 7.44 (br, 2H), 7.41-7.34 (m, 6H), 7.33-7.22 (m, 5H), 7.04-6.95 (m, 3H), 6.74 (d, J=8.4 Hz, 2H), 6.60 (d, J=9.3 Hz, 1H), 6.28 (d, J=8.6 Hz, 1H), 5.08 (p, J=7.1 Hz, 1H), 4.71 (t, J=8.1 Hz, 1H), 4.60 (d, J=8.7 Hz, 1H), 4.49 (br, 1H), 4.12 (d, J=11.5 Hz, 1H), 3.89 (br, 1H), 3.66 (dd, J=15.2, 9.1 Hz, 4H), 3.58 (dd, J=11.6, 3.5 Hz, 1H), 3.42 (br, 2H), 3.23 (br, 4H), 3.10 (dd, J=13.9, 5.0 Hz, 1H), 3.01 (dd, J=13.9, 7.2 Hz, 1H), 2.86 (br, 1H), 2.65-2.47 (m, 3H), 2.47-2.14 (m, 15H), 2.14-2.04 (m, 1H), 1.88 (br, 2H), 1.71-1.51 (m, 6H), 1.50-1.40 (m, 13H), 1.27 (br, 5H), 1.04 (s, 9H), 0.95 (s, 3H).

(2S,4R)-1-((S)-2-(8-(4-(((R)-4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #44): Degrader #44 was prepared from the aldehyde 1.12b by following the same protocol as degrader 17 was prepared from aldehyde 1.12. ¹H NMR (600 MHz, CDCl₃) δ 8.68 (s, 1H), 8.32 (d, J=2.3 Hz, 1H), 8.11 (d, J=9.2 Hz, 1H), 7.71 (br, 2H), 7.50 (d, J=7.8 Hz, 1H), 7.42-7.39 (m, 2H), 7.39-7.35 (m, 4H), 7.33-7.23 (m, 5H), 7.06-7.00 (m, 1H), 7.00-6.95 (m, 2H), 6.78 (d, J=8.8 Hz, 2H), 6.61 (d, J=9.3 Hz, 1H), 6.56 (d, J=9.3 Hz, 1H), 5.08 (p, J=7.1 Hz, 1H), 4.68 (t, J=8.2 Hz, 1H), 4.61 (d, J=9.2 Hz, 1H), 4.47 (s, 1H), 4.05 (d, J=11.4 Hz, 1H), 3.89 (br, 1H), 3.72-3.62 (m, 4H), 3.62-3.51 (m, 3H), 3.48-3.38 (m, 2H), 3.25 (br, 4H), 3.10 (dd, J=13.9, 5.0 Hz, 1H), 3.03 (dd, J=13.9, 7.2 Hz, 1H), 2.83 (br, 2H), 2.61-2.47 (m, 5H), 2.47-2.40 (m, 1H), 2.40-2.26 (m, 7H), 2.26-2.17 (m, 3H), 2.17-2.07 (m, 2H), 1.92 (d, J=17.4 Hz, 1H), 1.86-1.64 (m, 7H), 1.60 (p, J=14.8, 7.0 Hz, 5H), 1.50-1.41 (m, 5H), 1.31 (br, 5H), 1.03 (s, 9H), 0.95 (s, 3H).

### Example 36: Preparation of Degrader #45

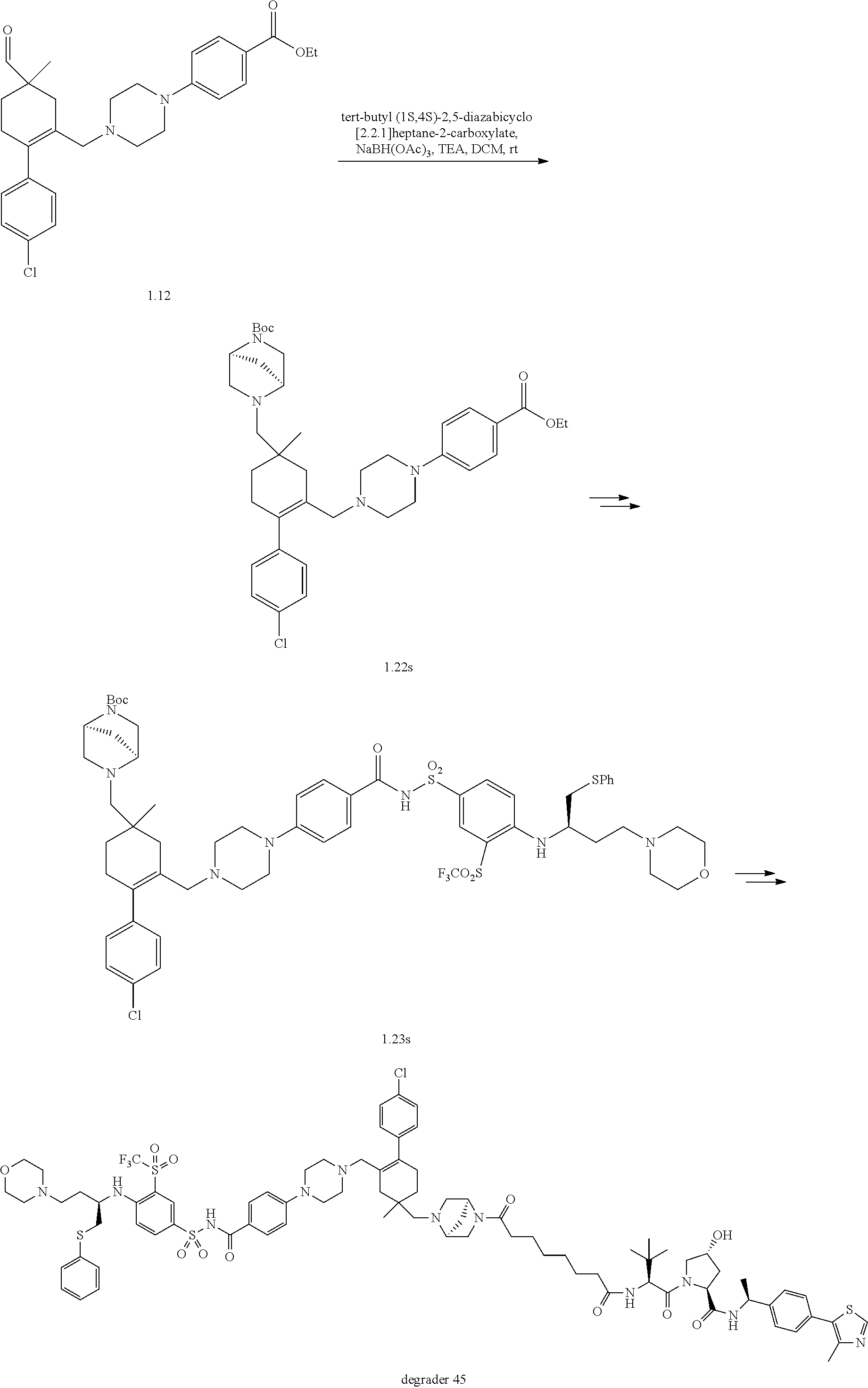

Preparation of tert-butyl (1S,4S)-5-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)-2,5-diazabicyclo[2.2.1]heptane-2-carboxylate (1.22s): Compound 1.22s was prepared from aldehyde 1.12 by following the same procedure as applied for the preparation of 1.22 starting with tert-butyl (1R,4R)-2,5-diazabicyclo[2.2.1]heptane-2-carboxylate. ¹H NMR (600 MHz, CDCl₃) δ 7.92-7.86 (m, 2H), 7.30-7.23 (m, 2H), 7.02-6.97 (m, 2H), 6.84-6.78 (m, 2H), 4.97 (br, 1H), 4.31 (q, J=7.1 Hz, 2H), 3.73 (br, 1H), 3.25 (t, J=5.2 Hz, 4H), 2.83-2.76 (m, 2H), 2.65 (br, 1H), 2.52 (br, 1H), 2.46-2.31 (m, 4H), 2.31-2.24 (m, OH), 2.24-2.14 (m, 2H), 2.11 (d, 1H), 1.91 (d, J=17.3 Hz, 1H), 1.67 (s, 1H), 1.63-1.49 (m, 7H), 1.43 (s, 9H), 1.36 (t, J=7.1 Hz, 3H), 0.94 (d, J=4.9 Hz, 3H).

Preparation of tert-butyl (1S,4S)-5-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,5-diazabicyclo[2.2.1]heptane-2-carboxylate (1.23s): Compound 1.23s was prepared from 1.22 by following the same procedure as compound 1.23 was prepared from 1.22. ¹H NMR (600 MHz, CDCl₃) δ 8.34 (s, 1H), 8.09 (d, J=9.2, 2.3 Hz, 1H), 7.67 (s, 2H), 7.36 (d, J=7.6 Hz, 2H), 7.27 (d, J=8.2 Hz, 6H), 7.02 (s, 1H), 6.98 (d, 2H), 6.75 (s, 2H), 6.58 (d, J=9.3 Hz, 1H), 4.97 (s, 1H), 3.88 (s, 1H), 3.79-3.49 (m, 5H), 3.27 (s, 4H), 3.09 (dd, J=13.8, 4.9 Hz, 1H), 3.01 (dd, J=13.9, 7.2 Hz, 1H), 2.86 (s, 2H), 2.68 (s, 1H), 2.55-2.03 (m, 11H), 1.93 (d, J=17.3 Hz, 1H), 1.77-1.33 (m, 20H), 0.94 (s, 3H).

(2S,4R)-1-((2S)-2-(8-((1S,4S)-5-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-2,5-diazabicyclo[2.2.1]heptan-2-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #45): ¹H NMR (600 MHz, CDCl₃) δ 8.66 (s, 1H), 8.30 (d, J=2.4 Hz, 1H), 8.05 (t, J=8.9 Hz, 1H), 7.77 (br, 2H), 7.48 (t, J=8.1 Hz, 1H), 7.40-7.30 (m, 6H), 7.31-7.20 (m, 3H), 7.00-6.92 (m, 3H), 6.75 (t, J=7.3 Hz, 2H), 6.56 (t, J=9.7 Hz, 1H), 6.46 (br, 1H), 5.11-5.01 (m, 1H), 4.72-4.62 (m, 2H), 4.62-4.52 (m, 1H), 4.47 (br, 1H), 4.21 (d, J=7.7 Hz, 1H), 4.08 (t, J=9.8 Hz, 1H), 3.86 (br, 1H), 3.69-3.59 (m, 5H), 3.59-3.51 (m, 1H), 3.51-3.37 (m, 2H), 3.32-3.10 (m, 6H), 3.08 (dd, J=13.8, 4.9 Hz, 1H), 2.99 (dd, J=13.6, 7.2 Hz, 1H), 2.90-2.68 (m, 3H), 2.54-2.47 (m, 4H), 2.46-2.03 (m, 9H), 1.96-1.74 (m, 3H), 1.73-1.62 (m, 2H), 1.62-1.48 (m, 7H), 1.45 (dd, J=17.8, 6.9 Hz, 3H), 1.41-1.28 (m, 4H), 1.24 (br, 7H), 1.02 (d, J=5.1 Hz, 9H), 0.90 (dd, J=7.2, 4.1 Hz, 3H).

### Example 37: Preparation of Degraders #46-48

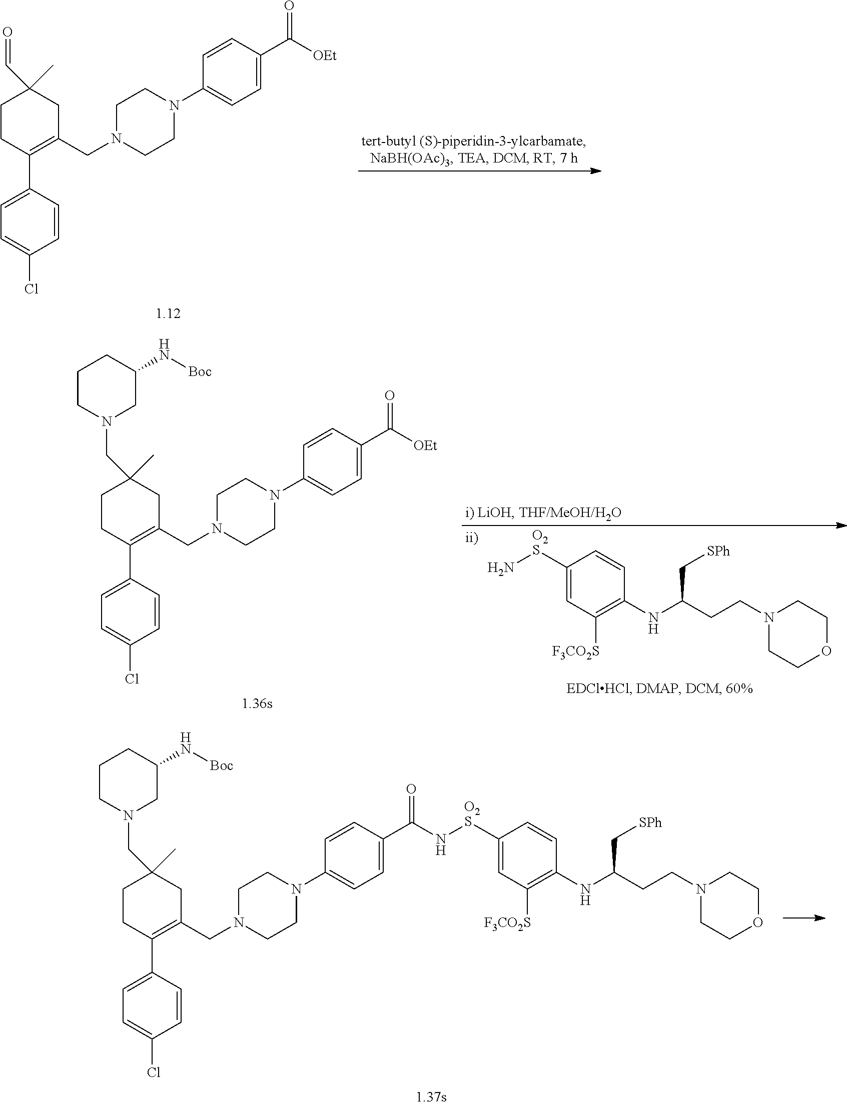

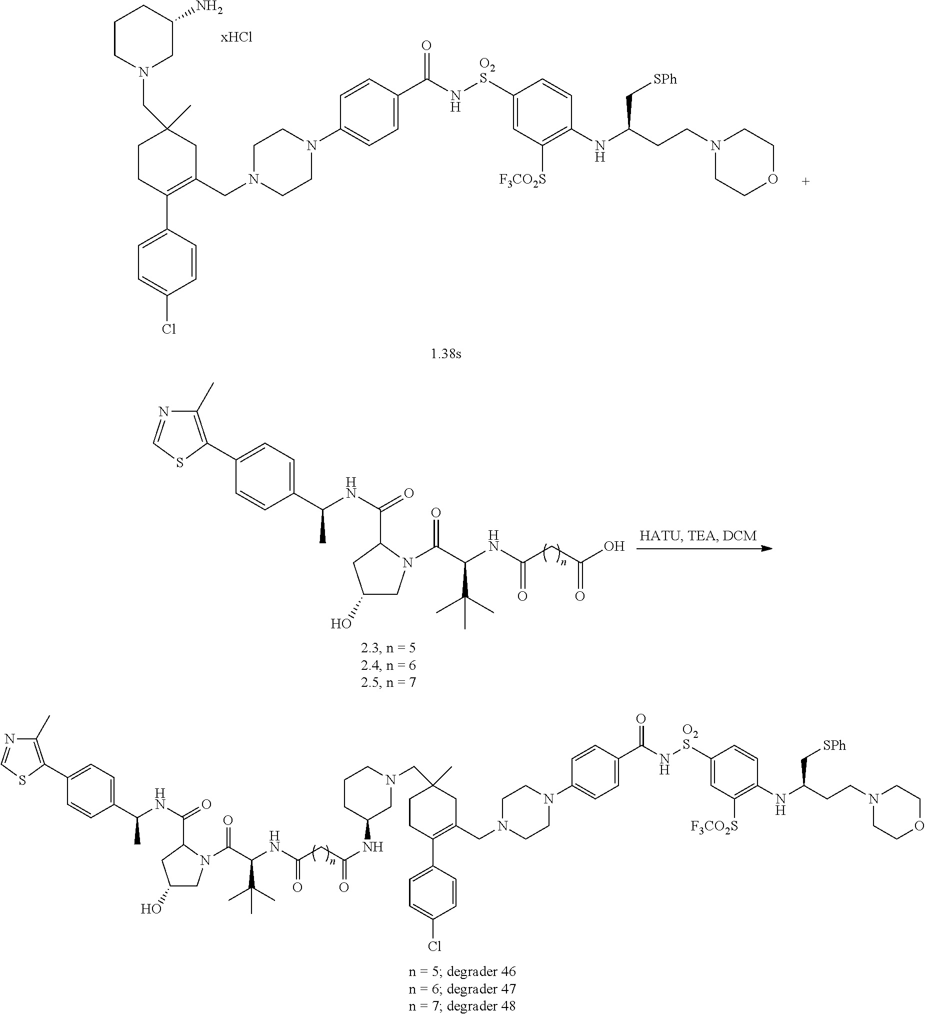

Preparation of ethyl 4-(4-((4-(((S)-3-((tert-butoxycarbonyl)amino)piperidin-1-yl)methyl)-4′-chloro-4-methyl-3,4,5,6-tetrahydro-[1,1′-biphenyl]-2-yl)methyl)piperazin-1-yl)benzoate (1.36s): To a stirring solution of aldehyde 1.12 (1.0 equiv.) in DCM was added tert-butyl (S)-piperidin-3-ylcarbamate (1.5 equiv.), NaBH(OAc)₃ (7.0 equiv.) and TEA (10 equiv.). The resulting mixture was stirred at room temperature for 7 h. After the completion of the reaction, the reaction mixture was diluted with DCM and then washed with water followed by brine. The organic portion was dried over anhydrous MgSO₄, filtered, and concentrated under reduced pressure. The crude product was purified by silica gel column chromatography to afford the title compound. ¹H NMR (600 MHz, CDCl₃) δ 7.92-7.86 (m, 2H), 7.29-7.24 (m, 2H), 7.01-6.97 (m, 2H), 6.81 (dt, J=9.8, 1.9 Hz, 2H), 4.97 (br, 1H), 4.31 (q, J=7.1 Hz, 2H), 3.72 (br, 1H), 3.25 (t, J=5.3 Hz, 4H), 2.85-2.76 (m, 2H), 2.70-2.58 (m, 1H), 2.51 (br, 1H), 2.45-2.30 (m, 4H), 2.31-2.14 (m, 2H), 2.11 (d, J=18.3, 3.0 Hz, 1H), 1.91 (d, J=17.3 Hz, 1H), 1.65 (d, J=15.4 Hz, 1H), 1.59 (s, 9H), 1.43 (s, 9H), 1.36 (t, J=7.1 Hz, 3H), 0.94 (d, J=4.9 Hz, 3H).

Preparation of tert-butyl ((3S)-1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-3-yl)carbamate (1.37s): Compound 1.37s was prepared from compound 1.36s by following the same procedure as compound 1.9 was prepared from the compound 1.8. ¹H NMR (600 MHz, CDCl₃) δ 8.34 (d, J=2.2 Hz, 1H), 8.07 (dd, J=9.2, 2.3 Hz, 1H), 7.69 (d, J=8.5 Hz, 2H), 7.36 (dt, J=6.2, 1.3 Hz, 2H), 7.32-7.20 (m, 7H), 7.03-6.93 (m, 3H), 6.73 (d, J=8.6 Hz, 2H), 6.57 (d, J=9.3 Hz, 1H), 4.98 (br, 1H), 3.92-3.80 (m, 1H), 3.76-3.57 (m, 5H), 3.25 (t, J=5.3 Hz, 4H), 3.09 (dd, J=13.9, 4.9 Hz, 1H), 3.00 (dd, J=13.9, 7.2 Hz, 1H), 2.86 (s, 2H), 2.59 (d, J=94.4 Hz, 2H), 2.48-2.24 (m, 11H), 2.24-2.06 (m, 3H), 1.92 (d, J=17.2 Hz, 1H), 1.72-1.62 (m, 2H), 1.62-1.49 (m, 1H), 1.49-1.32 (m, 11H), 0.93 (d, J=6.0 Hz, 3H).

Preparation of 4-(4-((4-(((S)-3-aminopiperidin-1-yl)methyl)-4′-chloro-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide hydrochloride (1.38s): Compound 1.38s was prepared from compound 1.37s by following the same procedure as compound 1.10 was prepared from compound 1.9.

General procedure for the preparation of degraders #46-48: Degraders #46-48 was prepared following the same procedure as that of degrader 1 with amine 1.38s in place of amine 1.10.

N1-((3S)-1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-3-yl)-N7-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)heptanediamide (degrader #46): ¹H NMR (600 MHz, CDCl₃) δ 8.66 (s, 1H), 8.30 (s, 1H), 8.07 (br, 1H), 7.74 (br, 2H), 7.43-7.31 (m, 8H), 7.31-7.18 (m, 4H), 6.98 (d, J=8.1 Hz, 3H), 6.74 (t, J=7.7 Hz, 2H), 6.58 (d, J=9.3 Hz, 1H), 6.48 (br, 1H), 5.07 (p, J=7.1 Hz, 1H), 4.71 (t, J=8.2, 2.0 Hz, 1H), 4.64 (d, 1H), 4.48 (br, 1H), 4.14-4.06 (m, 1H), 4.03 (br, 2H), 3.87 (br, 1H), 3.72-3.50 (m, 8H), 3.22 (br, 5H), 3.14-3.04 (m, 2H), 3.00 (dd, J=13.8, 7.2 Hz, 1H), 2.83 (br, 2H), 2.62 (br, 1H), 2.50 (s, 3H), 2.46-2.16 (m, 3H), 2.15-2.02 (m, 10H), 1.76-1.60 (m, 3H), 1.60-1.31 (m, 15H), 1.31-1.07 (m, 8H), 1.02 (s, 9H), 0.96 (s, 3H).

N1-((3S)-1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-3-yl)-N8-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)octanediamide (degrader #47): ¹H NMR (600 MHz, CDCl₃) δ 8.65 (s, 1H), 8.30 (s, 1H), 8.07 (s, 1H), 7.80-7.66 (m, 2H), 7.43 (s, 1H), 7.39-7.30 (m, 6H), 7.31-7.17 (m, 7H), 6.97 (d, J=8.1 Hz, 2H), 6.78-6.69 (m, 2H), 6.58 (d, J=9.3 Hz, 1H), 6.50 (s, 1H), 5.06 (q, J=6.7, 6.1 Hz, 1H), 4.72-4.58 (m, 2H), 4.47 (s, 1H), 4.09-3.99 (m, 2H), 3.86 (s, 1H), 3.70-3.49 (m, 6H), 3.22 (s, 4H), 3.08 (ddd, J=8.8, 5.4, 2.4 Hz, 3H), 3.00 (dd, J=13.8, 7.2 Hz, 1H), 2.82 (s, 2H), 2.49 (d, J=3.1 Hz, 2H), 2.45-2.24 (m, 16H), 2.09 (d, J=10.9 Hz, 5H), 1.66 (dd, J=14.3, 7.5 Hz, 1H), 1.49-1.33 (m, 16H), 1.22-1.13 (m, 6H), 1.02 (s, 7H), 0.95 (d, J=4.1 Hz, 3H).

N1-((3S)-1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-3-yl)-N9-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)nonanediamide (degrader #48): ¹H NMR (600 MHz, CDCl₃) δ 8.65 (s, 1H), 8.30 (s, 1H), 8.07 (d, J=9.1 Hz, 1H), 7.75 (br, 2H), 7.42 (br, 1H), 7.39-7.31 (m, 6H), 7.31-7.19 (m, 5H), 6.98 (d, J=8.0 Hz, 3H), 6.73 (d, J=8.6 Hz, 2H), 6.59 (d, J=9.3 Hz, 1H), 6.52 (br, 1H), 5.06 (p, J=7.1 Hz, 1H), 4.72-4.58 (m, 2H), 4.47 (s, 1H), 4.07 (d, J=11.5 Hz, 1H), 3.87 (br, 1H), 3.69-3.60 (m, 10H), 3.57 (d, J=11.1 Hz, 1H), 3.26 (br, 4H), 3.09 (q, J=7.5 Hz, 8H), 3.00 (dd, J=13.8, 7.1 Hz, 1H), 2.50 (s, 3H), 2.46-2.22 (m, 13H), 2.22-2.04 (m, 7H), 1.71-1.29 (m, 13H), 1.22-1.09 (m, 2H), 1.02 (s, 13H).

### Example 38: Preparation of Degraders #49-51

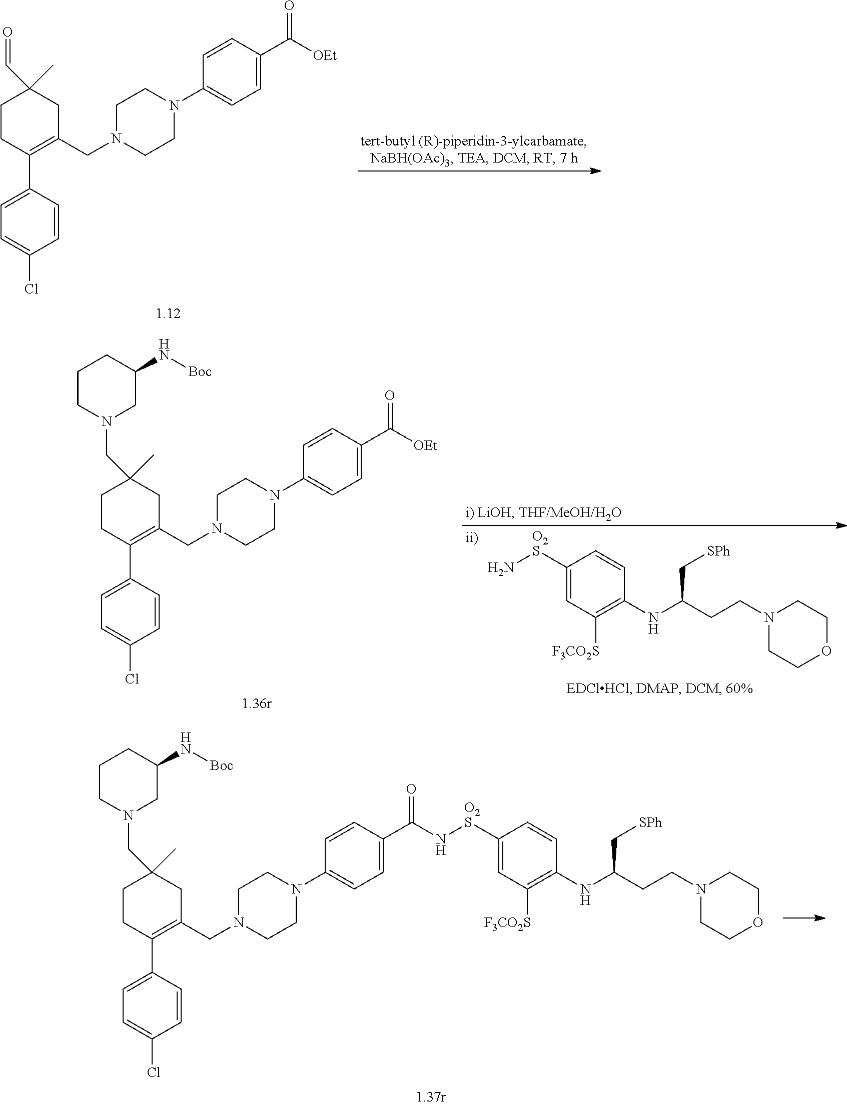

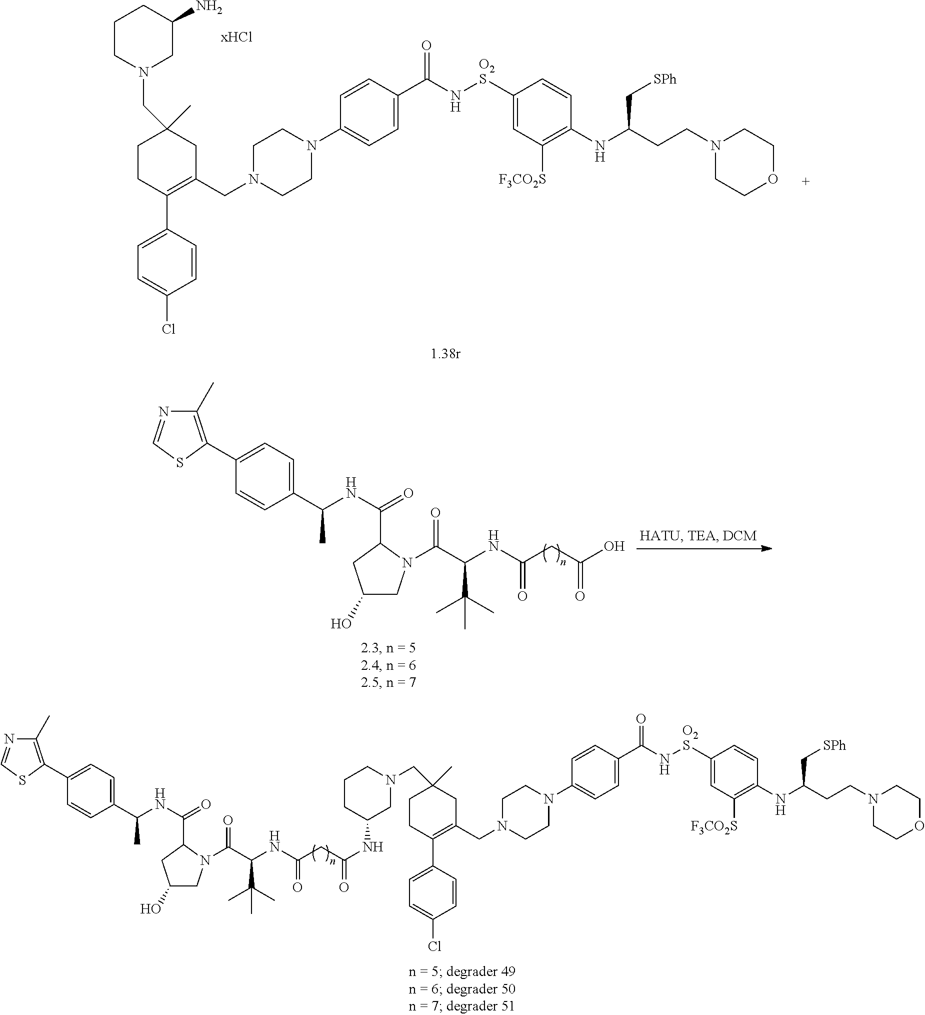

Degraders #49-51 were prepared from aldehyde 1.12 by following the same synthetic protocol as degrader #46 was prepared from aldehyde 1.12, with tert-butyl (R)-piperidin-3-ylcarbamate was used in place of tert-butyl (S)-piperidin-3-ylcarbamate in the synthetic sequence.

ethyl 4-(4-((4-(((R)-3-((tert-butoxycarbonyl)amino)piperidin-1-yl)methyl)-4′-chloro-4-methyl-3,4,5,6-tetrahydro-[1,1′-biphenyl]-2-yl)methyl)piperazin-1-yl)benzoate (1.36r): ¹H NMR (600 MHz, CDCl₃) δ 7.93-7.86 (m, 2H), 7.27 (d, J=8.1 Hz, 2H), 7.02-6.96 (m, 2H), 6.84-6.77 (m, 2H), 4.32 (q, J=7.1 Hz, 2H), 4.21 (s, 1H), 3.57-3.32 (m, 2H), 3.30-3.20 (m, 4H), 3.17 (d, J=11.4 Hz, 1H), 3.11-2.98 (m, 1H), 2.80 (s, 2H), 2.69-2.53 (m, 1H), 2.50-2.40 (m, 2H), 2.40-2.15 (m, 4H), 2.14-2.05 (m, 1H), 1.92 (d, J=17.6 Hz, 1H), 1.81 (d, J=9.1 Hz, 1H), 1.72-1.61 (m, 1H), 1.60-1.51 (m, 4H), 1.46 (s, 9H), 1.36 (t, J=7.1 Hz, 3H), 0.92 (d, J=4.3 Hz, 3H).

tert-butyl ((3R)-1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-3-yl)carbamate (1.37r): ¹H NMR (600 MHz, CDCl₃) δ 8.33 (d, J=2.4 Hz, 1H), 8.07 (d, J=9.0, 2.3 Hz, 1H), 7.70 (d, J=8.5 Hz, 2H), 7.40-7.32 (m, 2H), 7.32-7.19 (m, 6H), 7.03-6.92 (m, 3H), 6.72 (d, J=8.5 Hz, 2H), 6.56 (d, J=9.3 Hz, 1H), 4.38-4.17 (m, 1H), 3.87 (br, 1H), 3.72-3.57 (m, 4H), 3.56-3.36 (m, 2H), 3.23 (br, 4H), 3.19-3.11 (m, 1H), 3.09 (dd, J=13.8, 4.9 Hz, 1H), 3.00 (dd, J=13.8, 7.1 Hz, 1H), 2.86 (br, 2H), 2.81-2.55 (m, 1H), 2.55-2.05 (m, 15H), 2.00-1.88 (m, 1H), 1.86-1.77 (m, 1H), 1.72-1.61 (m, 2H), 1.61-1.51 (m, 1H), 1.49-1.36 (m, 8H), 0.92 (s, 3H).

N1-((3R)-1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-3-yl)-N7-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)heptanediamide (degrader #49): ¹H NMR (600 MHz, CDCl₃) δ 8.66 (s, 1H), 8.30 (t, J=2.3 Hz, 1H), 8.05 (d, J=8.7 Hz, 1H), 7.75 (d, J=8.3 Hz, 2H), 7.42-7.32 (m, 7H), 7.32-7.19 (m, 5H), 7.02-6.90 (m, 2H), 6.78-6.67 (m, 2H), 6.59 (d, J=9.3 Hz, 1H), 6.52 (br, 1H), 5.06 (p, J=7.1 Hz, 1H), 4.68 (t, J=8.2 Hz, 1H), 4.61-4.55 (m, 1H), 4.48 (br, 1H), 4.07 (d, J=11.5 Hz, 1H), 4.02 (br, 1H), 3.87 (br, 1H), 3.71-3.60 (m, 5H), 3.58 (dt, J=11.4, 3.3 Hz, 1H), 3.46 (s, 1H), 3.21 (br, 4H), 3.13 (q, J=7.5 Hz, 1H), 3.09 (dd, J=13.8, 4.9 Hz, 1H), 3.00 (dd, J=13.8, 7.2 Hz, 1H), 2.87 (br, 1H), 2.64 (br, 1H), 2.50 (s, 3H), 2.47-2.18 (m, 12H), 2.08 (tq, J=16.5, 8.6 Hz, 7H), 1.81-1.42 (m, 9H), 1.42-1.31 (m, 5H), 1.16 (br, 3H), 1.02 (s, 9H), 0.97 (s, 3H).

N1-((3R)-1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-3-yl)-N8-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)octanediamide (degrader #50): ¹H NMR (600 MHz, CDCl₃) δ 8.65 (s, 1H), 8.28 (d, J=1.9 Hz, 1H), 8.03 (d, J=8.7, 2.9 Hz, 1H), 7.77 (d, J=8.5 Hz, 2H), 7.50-7.40 (m, 1H), 7.39-7.31 (m, 6H), 7.30-7.24 (m, 3H), 7.24-7.19 (m, 1H), 6.98 (d, J=8.0 Hz, 2H), 6.92 (d, J=8.0 Hz, 1H), 6.72 (br, 2H), 6.60 (td, J=20.4, 9.0 Hz, 1H), 5.05 (p, J=7.1 Hz, 1H), 4.64 (t, J=8.2 Hz, 1H), 4.58 (t, J=8.0 Hz, 1H), 4.46 (s, 1H), 4.09-3.96 (m, 2H), 3.86 (br, 1H), 3.70-3.59 (m, 5H), 3.59-3.54 (m, 1H), 3.44 (s, 1H), 3.20 (br, 4H), 3.13 (q, J=7.4 Hz, 1H), 3.08 (dd, J=13.8, 5.0 Hz, 1H), 3.00 (dd, J=13.8, 7.1 Hz, 1H), 2.88 (br, 1H), 2.49 (s, 3H), 2.46-2.18 (m, 12H), 2.18-1.99 (m, 4H), 1.97-1.61 (m, 12H), 1.61-1.32 (m, 12H), 1.15 (d, J=14.1 Hz, 4H), 1.01 (s, 9H), 0.98 (s, 3H).

N1-((3R)-1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperidin-3-yl)-N9-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)nonanediamide (degrader #51): 1H NMR (600 MHz, CDCl₃) δ 8.66 (d, J=2.4 Hz, 1H), 8.29 (s, 1H), 8.06 (s, 1H), 7.78 (s, 2H), 7.36 (qd, J=8.1, 4.2 Hz, 7H), 7.29 (t, J=7.7 Hz, 4H), 7.23 (t, J=7.3 Hz, 1H), 7.04-6.93 (m, 3H), 6.72 (s, 2H), 6.60 (s, 1H), 6.35 (d, J=47.0 Hz, 1H), 5.08 (q, J=6.8 Hz, 1H), 4.69 (q, J=7.9 Hz, 1H), 4.58 (s, 1H), 4.49 (s, 1H), 4.12 (d, J=6.9 Hz, 1H), 4.04 (s, 1H), 3.88 (s, 1H), 3.73-3.54 (m, 7H), 3.22 (d, J=20.1 Hz, 4H), 3.15 (q, J=7.5 Hz, 2H), 3.09 (dd, J=13.8, 5.0 Hz, 1H), 3.01 (t, J=6.7 Hz, 1H), 2.51 (d, J=1.3 Hz, 3H), 2.47-2.21 (m, 19H), 2.09 (t, J=7.5 Hz, 9H), 1.66 (dd, J=13.6, 7.6 Hz, 3H), 1.46 (t, J=7.5 Hz, 8H), 1.43 (s, 6H), 1.13 (s, 6H), 1.04 (s, 9H), 0.97 (s, 3H).

### Example 39: Preparation of Degraders #52-54

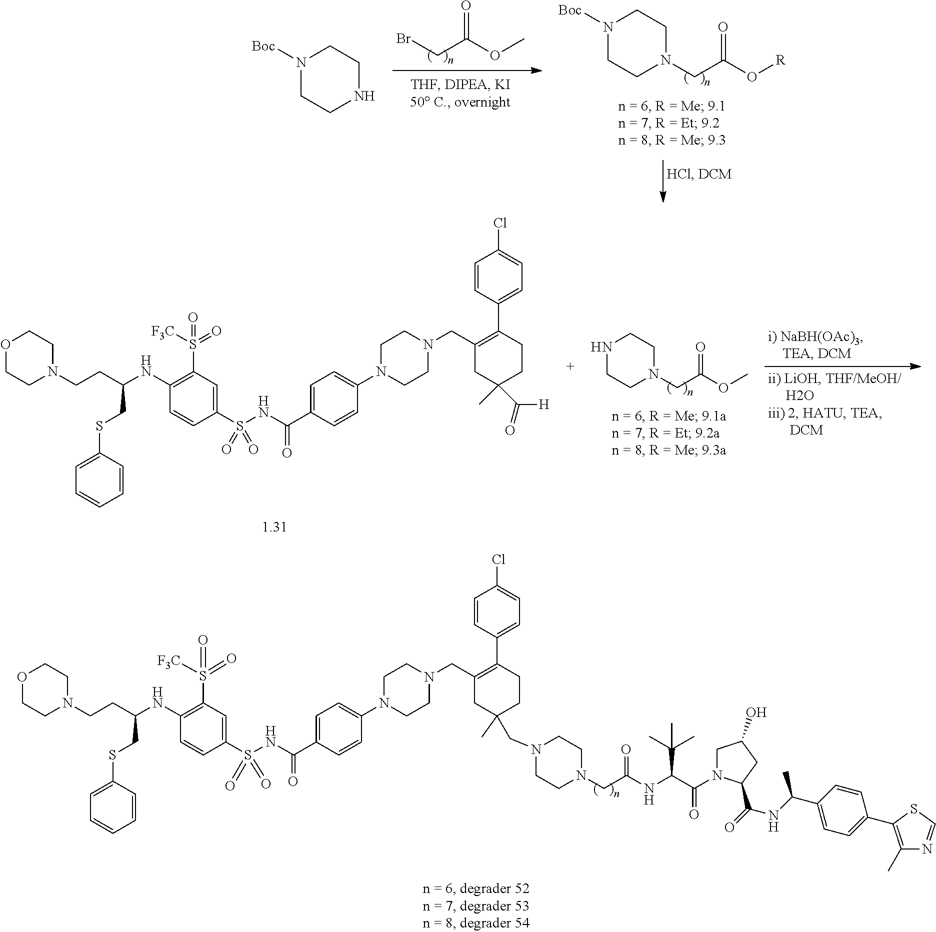

General procedure for the preparation of 9.1a-9.3a: To a stirring solution of tert-butyl piperazine-1-carboxylate (1.0 equiv.) in THF was added consecutively w-bromo ester (1.2 equiv.), KI (10 mol %) and DIPEA (2 equiv.). The mixture was stirred overnight at 50° C. THF was removed under reduced pressure and the residue was diluted with ethyl acetate. The organic portion was washed with water followed by brine, dried over anhydrous MgSO₄, filtered, and concentrated under reduced pressure. The crude product was purified by silica gel column chromatography to afford the corresponding esters 9.1-9.3.

Ester (9.1/9.2/9.3) was dissolved in DCM followed by the addition of HCl (10 equiv.) in dioxane. Once the reaction was complete, the volatiles were removed under reduced pressure to afford the corresponding title compounds, which were used in the next step without further purification.

tert-butyl 4-(7-methoxy-7-oxoheptyl)piperazine-1-carboxylate (9.1): ¹H NMR (600 MHz, CDCl₃) δ 3.66 (s, 3H), 3.42 (t, J=5.1 Hz, 4H), 2.36 (t, J=5.0 Hz, 4H), 2.31 (q, J=7.3 Hz, 4H), 1.67-1.57 (m, 2H), 1.52-1.46 (m, 2H), 1.46 (s, 9H), 1.38-1.26 (m, 4H).

tert-butyl 4-(8-ethoxy-8-oxooctyl)piperazine-1-carboxylate (9.2): ¹H NMR (600 MHz, CDCl3) δ 4.12 (q, J=7.1 Hz, 2H), 3.43 (t, J=5.1 Hz, 4H), 2.36 (t, J=5.1 Hz, 4H), 2.33-2.25 (m, 4H), 1.67-1.57 (m, 2H), 1.51-1.46 (m, 2H), 1.45 (s, 9H), 1.34-1.27 (m, 6H), 1.25 (t, J=7.1 Hz, 3H).

tert-butyl 4-(9-methoxy-9-oxononyl)piperazine-1-carboxylate (9.3): ¹H NMR (600 MHz, CDCl₃) δ 3.66 (s, 3H), 3.43 (t, J=5.1 Hz, 4H), 2.36 (t, J=5.0 Hz, 4H), 2.33-2.26 (m, 4H), 1.65-1.57 (m, 2H), 1.46 (s, 11H), 1.33-1.23 (m, 8H).

General procedure for the preparation of degraders #52-54: To a stirring solution of aldehyde 1.12 (1.0 equiv) in DCM was added amine 9.1a/9.2a/9.3a (1.0 equiv.), NaBH(OAc)₃ (7.0 equiv.) and TEA (10 equiv.). The resulting mixture was stirred at room temperature for 8 h. After the completion of the reaction, the reaction mixture was diluted with DCM and then washed with water followed by brine. The organic portion was dried over anhydrous MgSO₄, filtered, and concentrated under reduced pressure. The crude product was used in the next step without further purification.

To a stirring solution of the above crude product in MeOH/THF (1/1) was added aqueous LiOH.H₂O (3.0 equiv.) solution and the mixture was stirred for 10 h at room temperature. Once the starting material was consumed, the pH of the reaction was adjusted to 6.0 using 1N HCl. Organic solvents were removed from the mixture and the residue was diluted with EtOAc. The organic portion was washed with water and brine, dried over anhydrous Na₂SO₄, filtered, and concentrated under reduced pressure. The crude powder was used in the next step without further purification.

A mixture of amine 9.1a/9.2a/9.3a (1.0 equiv.), crude acid from above (1.1 equiv.), HATU (1.2 equiv.) and TEA (5.0 equiv.) was taken in DCM and the reaction mixture was stirred at room temperature for 4 h. After the reaction was completed, the mixture was diluted with DCM and washed with saturated aqueous NH₄Cl solution. The organic portion was dried over anhydrous MgSO₄, filtered, and concentrated under reduced pressure. The crude product was purified by silica gel column chromatography.

(2S,4R)-1-((2S)-2-(7-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)heptanamido)-3,3-dimethylbutanoyl)-4-hydroxy-N—((S)-1-(4-(4-methylthiazol-5-yl)phenyl)ethyl)pyrrolidine-2-carboxamide (degrader #52): ¹H NMR (600 MHz, CDCl₃) δ 8.65 (s, 1H), 8.33 (s, 1H), 7.96 (d, J=9.0 Hz, 1H), 7.88 (d, J=8.5 Hz, 2H), 7.45 (br, 1H), 7.38-7.30 (m, 6H), 7.27 (s, 3H), 6.97 (d, J=8.4, 1.1 Hz, 2H), 6.83 (d, J=8.5 Hz, 1H), 6.76 (d, J=8.6 Hz, 2H), 6.64 (br, 1H), 6.52 (d, J=9.3 Hz, 1H), 5.06 (p, 1H), 4.69 (t, J=8.2 Hz, 1H), 4.59 (d, J=8.9 Hz, 1H), 4.45 (br, 1H), 4.04 (d, J=11.4 Hz, 1H), 3.87-3.77 (m, 1H), 3.69-3.58 (m, 4H), 3.55 (d, J=10.8 Hz, 1H), 3.25-3.10 (m, 4H), 3.07 (dd, J=13.8, 4.8 Hz, 1H), 2.95 (dd, J=13.8, 7.5 Hz, 1H), 2.91-2.70 (m, 8H), 2.50 (s, 3H), 2.44-2.25 (m, 4H), 2.25-2.03 (m, 5H), 1.84 (t, J=15.6 Hz, 1H), 1.67-1.47 (m, 2H), 1.47-1.37 (m, 3H), 1.32-1.13 (m, 28H), 1.02 (s, 9H), 0.91 (d, J=3.1 Hz, 3H).

(2S,4R)-1-((2S)-2-(8-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)octanamido)-3,3-dimethylbutanoyl)-4-hydroxy-N—((S)-1-(4-(4-methylthiazol-5-yl)phenyl)ethyl)pyrrolidine-2-carboxamide (degrader #53): ¹H NMR (600 MHz, CDCl₃) δ 8.65 (s, 1H), 8.28 (d, J=2.5 Hz, 1H), 7.94 (d, J=9.4 Hz, 1H), 7.83 (d, J=8.6 Hz, 1H), 7.42-7.30 (m, 5H), 7.30-7.17 (m, 2H), 6.99 (d, J=7.7 Hz, 2H), 6.85 (d, J=8.5 Hz, 1H), 6.74 (d, J=8.6 Hz, 1H), 6.66-6.49 (m, 1H), 5.05 (p, J=7.6 Hz, 1H), 4.64 (t, J=8.3 Hz, 1H), 4.48 (br, 2H), 4.08 (d, J=11.5 Hz, 1H), 3.85 (br, 1H), 3.68-3.52 (m, 5H), 3.42 (s, 1H), 3.28-3.04 (m, 5H), 3.04-2.63 (m, 6H), 2.50 (s, 3H), 2.45-2.10 (m, 5H), 2.01-1.69 (m, 26H), 1.69-1.10 (m, 22H), 1.02 (d, J=1.9 Hz, 9H), 0.91 (s, 3H).

(2S,4R)-1-((2S)-2-(9-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)nonanamido)-3,3-dimethylbutanoyl)-4-hydroxy-N—((S)-1-(4-(4-methylthiazol-5-yl)phenyl)ethyl)pyrrolidine-2-carboxamide (degrader #54): ¹H NMR (600 MHz, CDCl₃) δ 8.65 (s, 1H), 8.31 (d, J=2.2 Hz, 1H), 7.97 (d, J=9.1, 2.1 Hz, 1H), 7.87 (d, J=8.5 Hz, 2H), 7.42-7.30 (m, 7H), 7.30-7.17 (m, 4H), 6.96 (d, 2H), 6.83 (d, J=8.5 Hz, 1H), 6.76 (d, J=8.6 Hz, 2H), 6.55-6.43 (m, 2H), 5.06 (p, J=7.1 Hz, 1H), 4.66 (t, 1H), 4.58 (d, J=8.9 Hz, 1H), 4.45 (br, 1H), 4.04 (d, J=11.4 Hz, 1H), 3.81 (br, 1H), 3.69-3.58 (m, 4H), 3.56 (dd, J=11.6, 3.4 Hz, 1H), 3.42 (p, J=1.6 Hz, 1H), 3.25-3.10 (m, 4H), 3.07 (dd, J=13.8, 4.7 Hz, 1H), 2.95 (dd, J=13.8, 7.5 Hz, 1H), 2.87-2.66 (m, 7H), 2.50 (s, 3H), 2.45-2.24 (m, 4H), 2.24-2.14 (m, 5H), 2.09 (d, J=13.6 Hz, 2H), 1.84 (d, J=17.3 Hz, 1H), 1.69-1.29 (m, 25H), 1.29-1.15 (m, 9H), 1.01 (s, 9H), 0.90 (s, 3H).

### Example 40: Preparation of Degraders #55 and #56

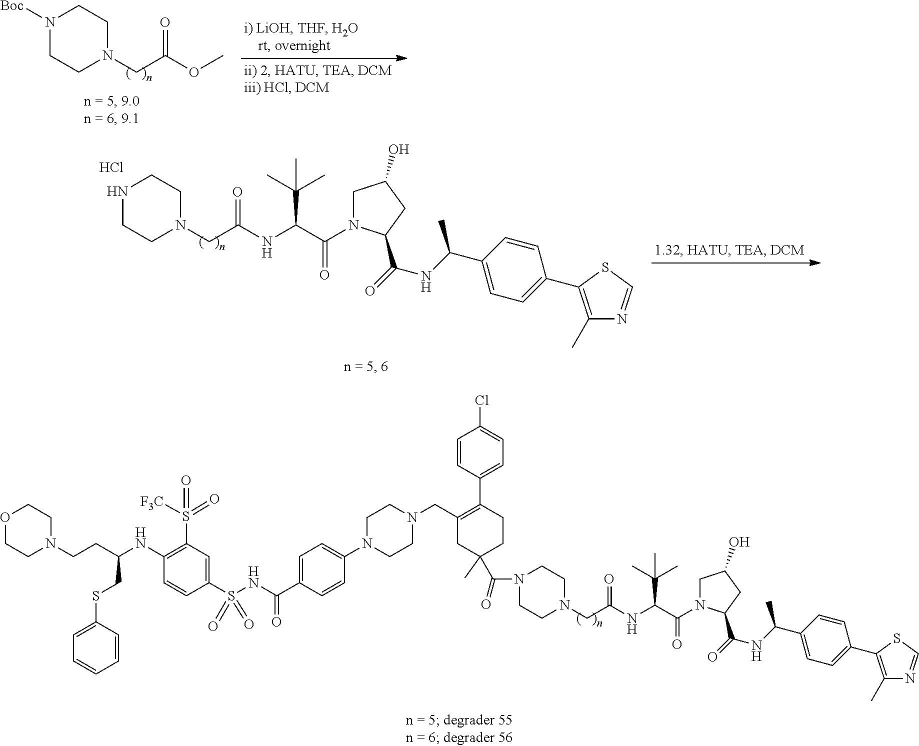

Preparation of tert-butyl 4-(6-methoxy-6-oxohexyl)piperazine-1-carboxylate (9.0): Compound 9.0 was prepared by following the same procedure as compound 9.1 was synthesized from tert-butyl piperazine-1-carboxylate. ¹H NMR (600 MHz, CDCl₃) δ 3.66 (s, 3H), 3.42 (t, J=5.1 Hz, 4H), 2.36 (t, J=5.0 Hz, 4H), 2.32 (q, J=7.1 Hz, 4H), 1.68-1.60 (m, 2H), 1.53-1.47 (m, 2H), 1.45 (s, 9H), 1.37-1.30 (m, 2H).

General procedure for the preparation of degraders #55 and #56: To a stirring solution of ester 9.0 or 9.1 in MeOH/THF (1/1) was added LiOH.H₂O (3 equiv.) solution in H₂O and the mixture was stirred for 8 h at room temperature. Once the starting material was consumed, the pH of the reaction was adjusted to 6.0 using 1N HCl. Organic solvents were removed from the mixture and crude was diluted with EtOAc. The organic portion was washed with water and brine, dried over anhydrous Na₂SO₄, filtered, and the solvent was removed under reduced pressure. A mixture of crude acid (1.0 equiv), amine 2.0 (1.1 euiv), HATU (1.2 equiv) and TEA (5.0 equiv) was taken in DCM and the reaction mixture was stirred at room temperature for 4 h. After the reaction was completed, the mixture was diluted with DCM and washed with saturated aqueous NH₄Cl solution. The organic portion was dried over anhydrous MgSO₄, filtered, and concentrated under reduced pressure. The crude product was dissolved in DCM followed by the addition of HCl (10 equiv.) in dioxane. Once the reaction was completed, the volatiles were removed in reduced pressure to afford the corresponding amine salt.

A mixture of crude amine salt from above (1.0 equiv.), acid 1.32 (1.0 equiv.), HATU (1.2 equiv.) and TEA (5 equiv.) was taken in DCM and the reaction mixture was stirred at room temperature for 4 h. After the reaction was completed, the mixture was diluted with DCM and washed with saturated aqueous NH₄Cl solution. The organic portion was dried over anhydrous MgSO₄, filtered, and concentrated under reduced pressure. The crude product was purified by silica gel column chromatography.

(2S,4R)-1-((2S)-2-(6-(4-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carbonyl)piperazin-1-yl)hexanamido)-3,3-dimethylbutanoyl)-4-hydroxy-N—((S)-1-(4-(4-methylthiazol-5-yl)phenyl)ethyl)pyrrolidine-2-carboxamide (degrader #55): ¹H NMR (600 MHz, CDCl₃) δ 8.66 (s, 1H), 8.30 (s, 1H), 8.06 (br, 1H), 7.72 (br, 2H), 7.42-7.31 (m, 7H), 7.31-7.15 (m, 4H), 7.03-6.90 (m, 3H), 6.76 (d, J=8.6 Hz, 2H), 6.64-6.46 (m, 1H), 5.06 (q, J=7.0 Hz, 1H), 4.65 (t, J=8.0 Hz, 1H), 4.60 (t, J=8.3 Hz, 1H), 4.46 (br, 1H), 4.04 (d, J=11.5 Hz, 1H), 3.86 (br, 1H), 3.76-3.59 (m, 7H), 3.57 (dd, J=11.5, 3.3 Hz, 1H), 3.41 (p, J=1.6 Hz, 2H), 3.28-3.13 (m, 4H), 3.07 (dd, 1H), 3.00 (dd, 1H), 2.84 (d, J=12.8 Hz, 1H), 2.78 (d, J=12.7 Hz, 1H), 2.50 (s, 3H), 2.47-2.21 (m, 19H), 2.12 (d, J=25.3 Hz, 3H), 1.96 (s, 14H), 1.86-1.79 (m, 1H), 1.47 (d, J=6.9 Hz, 3H), 1.32 (s, 3H), 1.01 (d, J=3.2 Hz, 9H).

(2S,4R)-1-((2S)-2-(7-(4-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carbonyl)piperazin-1-yl)heptanamido)-3,3-dimethylbutanoyl)-4-hydroxy-N—((S)-1-(4-(4-methylthiazol-5-yl)phenyl)ethyl)pyrrolidine-2-carboxamide (degrader #56): ¹H NMR (600 MHz, CDCl₃) δ 8.66 (s, 1H), 8.29 (s, 1H), 8.02 (d, J=9.1 Hz, 1H), 7.79 (br, 2H), 7.41-7.30 (m, 7H), 7.24 (s, 4H), 7.00-6.93 (m, 2H), 6.90 (d, J=8.5 Hz, 1H), 6.75 (d, J=8.7 Hz, 2H), 6.53 (d, J=9.3 Hz, 1H), 6.48 (br, 1H), 5.06 (p, J=7.1 Hz, 1H), 4.68-4.62 (m, 1H), 4.60 (dd, J=9.3, 4.8 Hz, 1H), 4.46 (s, 1H), 4.04 (d, J=11.5 Hz, 1H), 3.87-3.79 (m, 1H), 3.76-3.59 (m, 12H), 3.56 (dd, J=11.4, 3.3 Hz, 1H), 3.41 (p, J=1.7 Hz, 1H), 3.26-3.15 (m, 4H), 3.08 (q, J=7.3 Hz, 5H), 2.97 (dd, J=13.8, 7.4 Hz, 1H), 2.88-2.74 (m, 3H), 2.50 (s, 3H), 2.47-2.22 (m, 5H), 2.22-2.13 (m, 3H), 2.13-2.05 (m, 2H), 1.96 (br, 6H), 1.87-1.80 (m, 5H), 1.78-1.68 (m, 1H), 1.68-1.36 (m, 4H), 1.32 (s, 3H), 1.29 (t, J=7.3 Hz, 6H), 1.01 (s, 9H).

### Example 41: Preparation of Degraders #57-59

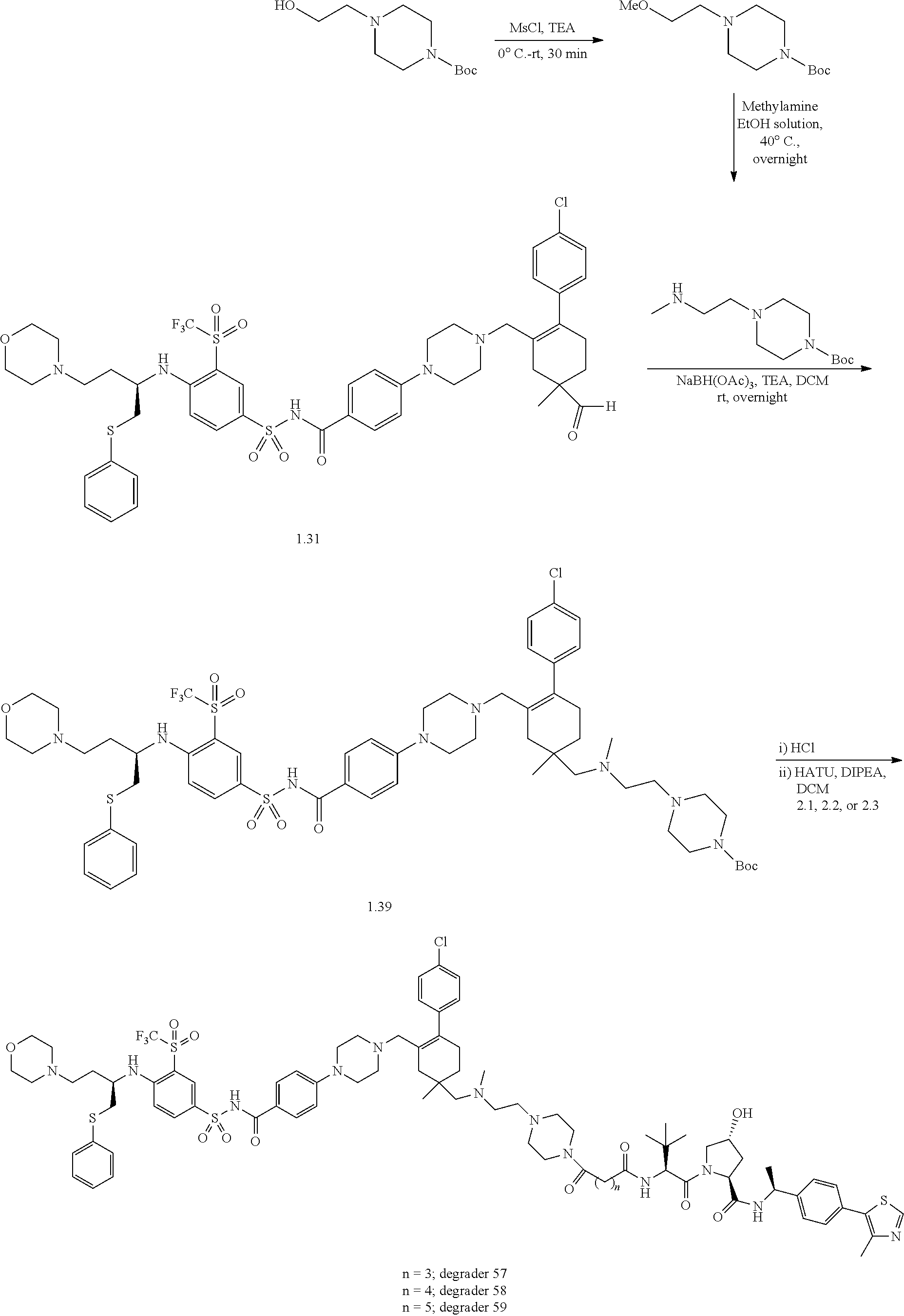

Preparation of tert-butyl 4-(2-(methylamino)ethyl)piperazine-1-carboxylate: To a stirring solution of tert-butyl 4-(2-hydroxyethyl)piperazine-1-carboxylate (1.0 equiv.) in DCM at 0° C. was added TEA (3.0 equiv.) followed by methanesulfonyl chloride (1.5 equiv.). The resulting mixture was stirred for 2 h at room temperature and then quenched with saturated aqueous NaHCO₃ solution. The mixture was diluted with DCM and the organic portion was washed with water followed by brine, dried over anhydrous Na₂SO₄, filtered, and concentrated under reduced pressure. The crude product was used in the next step without further purification.

The crude product from above was dissolved in ethanol followed by the addition of methyl amine solution (1.5 equiv.) in ethanol and the mixture was stirred at 40° C. overnight. After the reaction was complete, volatiles were evaporated in reduced pressure and the crude was purified by flash chromatography to get the title compound. 1H NMR (600 MHz, CDCl3) δ 3.48 (t, J=5.9, 4.2 Hz, 4H), 3.06 (t, J=6.0 Hz, 2H), 2.79 (t, J=6.0 Hz, 2H), 2.74 (s, 3H), 2.48 (t, J=5.1 Hz, 4H), 1.44 (s, 9H).

Preparation of tert-butyl 4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazine-1-carboxylate (1.39): To a stirring solution of aldehyde 1.31 (1.0 equiv.) in DCM was added tert-butyl 4-(2-(methylamino)ethyl)piperazine-1-carboxylate (1.5 equiv.), NaBH(OAc)₃ (5.0 equiv.) and TEA (10 equiv.). The resulting mixture was stirred at room temperature for 8 h. The reaction mixture was diluted with DCM and then washed with water followed by brine. The organic portion was dried over anhydrous MgSO₄, filtered, and concentrated under reduced pressure. The crude product was purified by silica gel column chromatography to afford the title compound. ¹H NMR (600 MHz, CDCl₃) δ 8.31 (d, J=2.2 Hz, 1H), 8.06 (d, J=9.1 Hz, 1H), 7.72 (d, J=8.5 Hz, 2H), 7.36 (dd, J=7.5, 1.7 Hz, 2H), 7.32-7.21 (m, 4H), 7.01-6.93 (m, 3H), 6.75 (d, J=8.8 Hz, 2H), 6.57 (d, J=9.3 Hz, 1H), 3.91-3.80 (m, 1H), 3.71-3.56 (m, 4H), 3.47-3.35 (m, 4H), 3.29-3.17 (m, 4H), 3.13-3.02 (m, 3H), 2.99 (dd, J=13.8, 7.3 Hz, 1H), 2.87-2.78 (m, 2H), 2.78-2.64 (m, 4H), 2.57 (s, 2H), 2.50-2.20 (m, 16H), 2.15-2.06 (m, 1H), 2.03-1.89 (m, 2H), 1.76-1.10 (m, 15H), 0.97 (s, 3H).

General procedure for the synthesis of degraders #57-59: To a stirring solution of compound 1.39 (1.0 equiv.) in DCM was added 4N HCl solution (10 equiv.) in dioxane and the mixture was stirred at room temperature for 5 h. Solvents were removed under reduced pressure and the remaining white power was washed with Et₂O and used in the next step without further purification.

To a stirring solution of crude amine salt from above (1.0 equiv.) and acid 2.1, 2.2, or 2.3 (1.1 equiv.) in DCM was added DIPEA (5.0 equiv.) at room temperature. To the mixture HATU (1.2 equiv.) was then added and the reaction were stirred for 8 h at the same temperature. Solvent was removed under reduced pressure and the crude product was purified by flash column chromatography (DCM/MeOH/TEA=96:7:1). The product from column was mixed with 15 mL DCM and washed with saturated aqueous NH₄Cl solution. The organic portion was dried over Na₂SO₄, filtered, and concentrated under reduced pressure to afford the corresponding degraders.

(2S,4R)-1-((2S)-2-(5-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide (degrader #57): ¹H NMR (600 MHz, CDCl₃) δ 8.66 (s, 1H), 8.31 (d, J=2.2 Hz, 1H), 8.06 (d, J=9.2 Hz, 1H), 7.77 (br, 2H), 7.49 (br, 1H), 7.41-7.31 (m, 6H), 7.31-7.17 (m, 4H), 7.06-6.89 (m, 3H), 6.75 (d, J=8.7 Hz, 2H), 6.56 (d, J=9.3 Hz, 1H), 5.06 (p, J=7.1 Hz, 1H), 4.70 (t, J=8.2 Hz, 1H), 4.53 (d, J=8.1 Hz, 1H), 4.47 (br, 1H), 4.12 (d, J=11.4 Hz, 1H), 3.85 (br, 1H), 3.70-3.59 (m, 4H), 3.57 (dd, J=11.3, 3.3 Hz, 1H), 3.50 (br, 3H), 3.33 (br, 2H), 3.18 (br, 4H), 3.13-3.03 (m, 1H), 3.03-2.91 (m, 1H), 2.86-2.75 (m, 2H), 2.63 (br, 2H), 2.53-2.46 (m, 5H), 2.46-2.14 (m, 22H), 2.14-2.04 (m, 2H), 1.91 (d, J=17.2 Hz, 1H), 1.85-1.73 (m, 2H), 1.69-1.51 (m, 2H), 1.50-1.30 (m, 1 OH), 1.04 (s, 9H), 0.92 (s, 3H).

(2S,4R)-1-((2S)-2-(6-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (degrader #58): ¹H NMR (600 MHz, CDCl₃) δ 8.65 (s, 1H), 8.30 (d, J=2.2 Hz, 1H), 8.05 (d, J=8.6 Hz, 1H), 7.76 (br, 2H), 7.42 (br, 1H), 7.39-7.31 (m, 6H), 7.31-7.18 (m, 2H), 7.00-6.91 (m, 2H), 6.81 (br, 1H), 6.75 (d, J=8.6 Hz, 2H), 6.56 (d, J=9.3 Hz, 1H), 5.05 (p, J=7.1 Hz, 1H), 4.66 (t, J=8.6 Hz, 1H), 4.59 (d, J=8.6 Hz, 1H), 4.46 (br, 1H), 4.07 (d, J=11.3 Hz, 1H), 3.85 (br, 1H), 3.71-3.52 (m, 5H), 3.52-3.38 (m, 3H), 3.38-3.26 (m, 2H), 3.19 (br, 4H), 3.12-3.03 (m, 1H), 3.03-2.92 (m, 1H), 2.86-2.73 (m, 2H), 2.68-2.54 (m, 2H), 2.54-1.99 (m, 22H), 1.89 (d, J=17.3 Hz, 1H), 1.83-1.48 (m, 15H), 1.48-1.28 (m, 10H), 1.03 (s, 9H), 0.91 (d, J=2.3 Hz, 3H).

(2S,4R)-1-((2S)-2-(7-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (degrader #59): ¹H NMR (600 MHz, CDCl₃) δ 8.65 (s, 1H), 8.30 (d, J=2.2 Hz, 1H), 8.01 (d, J=9.1 Hz, 1H), 7.81 (d, J=8.3 Hz, 2H), 7.43 (d, J=7.3 Hz, 1H), 7.39-7.30 (m, 6H), 7.31-7.16 (m, 4H), 6.98 (d, J=8.0 Hz, 2H), 6.88 (d, J=8.4 Hz, 1H), 6.72 (br, 1H), 6.66-6.49 (m, 2H), 5.06 (p, J=7.1 Hz, 1H), 4.67 (t, J=8.3 Hz, 1H), 4.51 (d, J=8.3 Hz, 1H), 4.47 (s, 1H), 4.11 (d, J=11.4 Hz, 1H), 3.85 (br, 1H), 3.69-3.58 (m, 4H), 3.55 (dd, J=11.4, 3.3 Hz, 1H), 3.48-3.34 (m, 2H), 3.29 (br, 2H), 3.16 (br, 3H), 3.08 (dd, J=13.8, 5.0 Hz, 1H), 2.98 (dd, J=13.6, 7.4 Hz, 1H), 2.87 (br, 2H), 2.62 (br, 2H), 2.56-2.14 (m, 29H), 2.14-2.02 (m, 3H), 1.97-1.80 (m, 1H), 1.73-1.27 (m, 15H), 1.27-1.13 (m, 2H), 1.02 (s, 9H), 0.93 (s, 3H).

### Example 42: Preparation of Degraders #60-62

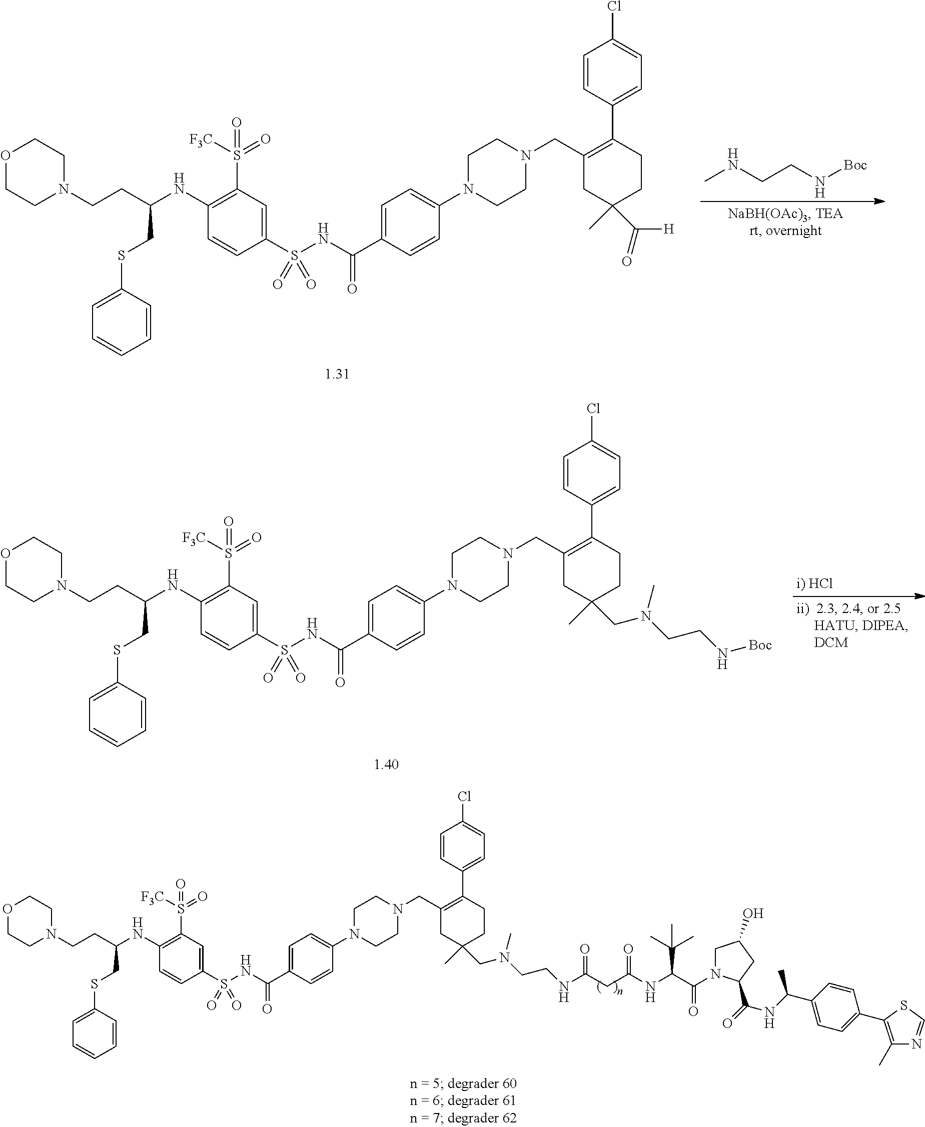

General procedure for the preparation of degraders #60-62: To a stirring solution of aldehyde 1.31 (1.0 equiv.) in DCM was added tert-butyl (2-(methylamino)ethyl)carbamate (1.2 equiv.), NaBH(OAc)₃ (5.0 equiv.) and TEA (10 equiv.). The resulting mixture was stirred at room temperature for 8 h then diluted with DCM. The mixture was washed with saturated aqueous NH₄Cl solution followed by brine. The organic portion was dried over anhydrous MgSO₄, filtered, and then concentrated under reduced pressure. The crude product 1.40 was dissolved in DCM followed by the addition of HCl in dioxane (10 equiv.). After completion of the reaction, the volatiles were removed under reduced pressure to yield a crude powder, which was used in the next step without further purification.

To a stirring solution of crude amine salt from above (1.0 equiv.) and acid 2.3, 2.4, or 2.5 (1.1 equiv.) in DCM was added DIPEA (5 equiv.) at room temperature. To the mixture HATU (1.2 equiv.) was added and the reaction were stirred for 8 h at the same temperature. Solvent was removed under reduced pressure and the crude product was purified by flash column chromatography (DCM:MeOH:TEA=96:7:1). The product from column was mixed with 15 mL DCM and washed with saturated aqueous NH₄Cl solution. The organic portion was dried over Na₂SO₄, filtered, and concentrated under reduced pressure to afford the corresponding degraders.

N1-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)-N7-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)heptanediamide (degrader #60): ¹H NMR (600 MHz, CDCl₃) δ 8.66 (s, 1H), 8.32 (d, J=2.2 Hz, 1H), 8.00 (d, J=9.1, 2.2 Hz, 1H), 7.88 (d, J=8.5 Hz, 2H), 7.43 (d, 1H), 7.40-7.32 (m, 6H), 7.30-7.19 (m, 5H), 7.01-6.92 (m, 2H), 6.80 (d, J=8.5 Hz, 1H), 6.74 (d, J=9.0 Hz, 2H), 6.49 (d, J=9.2 Hz, 1H), 6.43 (d, J=8.8 Hz, 1H), 5.07 (p, J=7.1 Hz, 1H), 4.70 (t, J=8.1 Hz, 1H), 4.58 (d, J=8.9 Hz, 1H), 4.47 (s, 1H), 4.05 (d, J=11.3 Hz, 1H), 3.86-3.76 (m, 1H), 3.69-3.58 (m, 5H), 3.56 (dd, J=11.5, 3.5 Hz, 1H), 3.36-3.21 (m, 2H), 3.17 (t, J=5.3 Hz, 4H), 3.12-3.01 (m, 3H), 2.94 (dd, J=13.8, 7.6 Hz, 1H), 2.79 (s, 2H), 2.56 (t, J=6.1 Hz, 2H), 2.51 (s, 3H), 2.46-2.24 (m, 10H), 2.24-2.04 (m, 6H), 2.03-1.94 (m, 1H), 1.67-1.58 (m, 1H), 1.58-1.48 (m, 9H), 1.46 (d, J=6.9 Hz, 3H), 1.35 (d, J=6.7 Hz, 6H), 1.27-1.15 (m, 2H), 1.01 (s, 9H), 0.96 (s, 3H).

N1-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)-N8-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)octanediamide (degrader #61): ¹H NMR (600 MHz, CDCl₃) δ 8.66 (s, 1H), 8.31 (d, J=2.2 Hz, 1H), 8.01 (d, J=9.1, 2.2 Hz, 1H), 7.85 (d, J=8.4 Hz, 2H), 7.51-7.44 (m, 1H), 7.41-7.31 (m, 6H), 7.31-7.17 (m, 4H), 6.97 (d, 2H), 6.84 (d, J=8.5 Hz, 1H), 6.74 (d, 2H), 6.55-6.43 (m, 2H), 5.07 (p, J=7.1 Hz, 1H), 4.68 (t, J=8.2 Hz, 1H), 4.59 (d, J=8.9 Hz, 1H), 4.47 (s, 1H), 4.07 (d, J=11.5 Hz, 1H), 3.82 (br, 1H), 3.70-3.58 (m, 5H), 3.56 (dd, J=11.4, 3.4 Hz, 1H), 3.36-3.22 (m, 2H), 3.22-3.12 (m, 4H), 3.11-3.03 (m, 2H), 2.96 (dd, J=13.8, 7.5 Hz, 1H), 2.79 (s, 2H), 2.56 (t, J=6.1 Hz, 2H), 2.50 (s, 3H), 2.46-2.24 (m, 12H), 2.24-1.92 (m, 7H), 1.63 (dq, J=14.0, 6.2 Hz, 1H), 1.58-1.41 (m, 12H), 1.37 (d, J=15.0, 7.0 Hz, 6H), 1.18 (s, 4H), 1.02 (s, 9H), 0.96 (s, 3H).

N1-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)-N9-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)nonanediamide (degrader #62): ¹H NMR (600 MHz, CDCl₃) δ 8.65 (s, 1H), 8.30 (d, J=2.2 Hz, 1H), 8.04 (d, 1H), 7.79 (d, J=8.4 Hz, 2H), 7.50-7.41 (m, 1H), 7.40-7.30 (m, 6H), 7.30-7.23 (m, 4H), 7.24-7.17 (m, 1H), 6.96 (d, 2H), 6.91 (d, J=8.6 Hz, 1H), 6.73 (d, J=8.7 Hz, 2H), 6.58-6.46 (m, 2H), 5.06 (p, J=7.1 Hz, 1H), 4.66 (t, J=8.2 Hz, 1H), 4.62 (d, J=9.1 Hz, 1H), 4.46 (s, 1H), 4.04 (d, J=11.3 Hz, 1H), 3.84 (br, 1H), 3.69-3.59 (m, 5H), 3.57 (dd, J=11.4, 3.4 Hz, 1H), 3.37-3.25 (m, 2H), 3.24-3.16 (m, 4H), 3.12-3.04 (m, 4H), 2.97 (dd, J=13.8, 7.3 Hz, 1H), 2.84 (br, 2H), 2.64 (br, 2H), 2.49 (s, 3H), 2.45-2.25 (m, 10H), 2.25-1.98 (m, 6H), 1.68-1.59 (m, 1H), 1.59-1.48 (m, 12H), 1.44 (d, J=6.9 Hz, 6H), 1.25-1.13 (m, 6H), 1.01 (s, 9H), 0.98 (s, 3H).

### Example 43: Preparation of Degraders #63-66

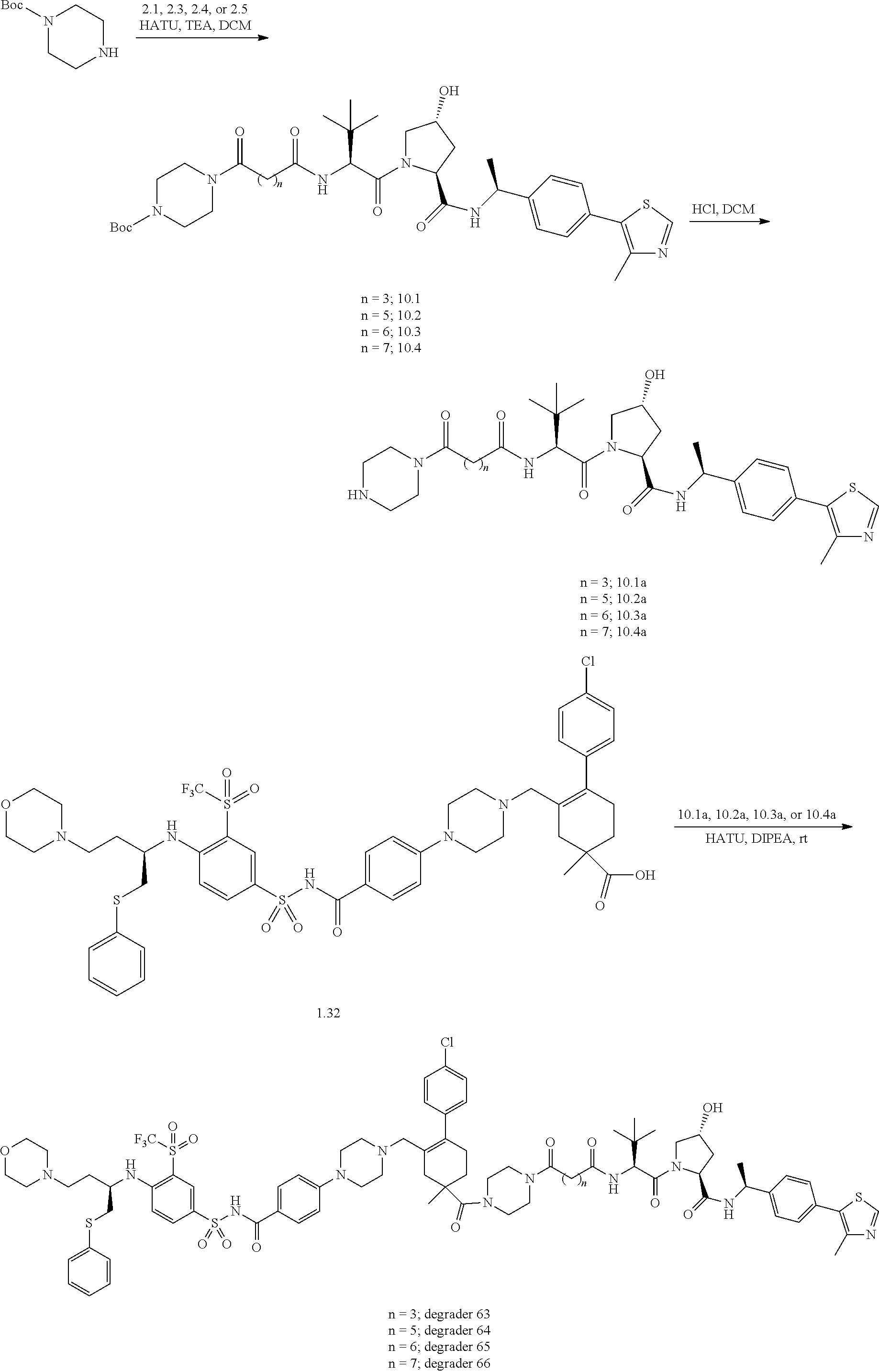

General procedure for the preparation of amine salts 10.1a-10.4a: A mixture of acid 2.1, 2.3, 2.4, or 2.5 (1.1 equiv.), tert-butyl piperazine-1-carboxylate (1.0 equiv.), HATU (1.2 equiv.) and TEA (5.0 equiv.) was taken in DCM and the reaction mixture was stirred at room temperature for 4 h. The mixture was diluted with DCM and washed with saturated aqueous NH₄Cl solution. The organic portion was dried over anhydrous MgSO₄, filtered, and concentrated under reduced pressure. The crude product was purified by flash column chromatography to afford 10.1, 10.2, 10.3, and 10.4, respectively.

Compound 10.1, 10.2, 10.3, or 10.4 was dissolved in DCM followed by the addition of HCl (10 equiv.) in dioxane. Once the reaction was complete, the volatiles were removed under reduced pressure to acquire the pure amine salt which was used in the next step without further purification.

tert-butyl 4-(5-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-5-oxopentanoyl)piperazine-1-carboxylate (10.1): ¹H NMR (600 MHz, CDCl₃) δ 8.67 (s, 1H), 7.50 (d, J=7.9 Hz, 1H), 7.40 (d, 2H), 7.36 (d, 2H), 6.79 (br, 1H), 5.08 (p, J=7.1 Hz, 1H), 4.72 (t, J=8.0 Hz, 1H), 4.54-4.44 (m, 2H), 4.11 (d, J=11.7, 1.8 Hz, 1H), 3.63-3.50 (m, 4H), 3.47 (d, J=4.1 Hz, 1H), 3.45-3.33 (m, 6H), 2.52 (s, 3H), 2.52-2.46 (m, 1H), 2.46-2.21 (m, 4H), 2.11-2.03 (m, 1H), 1.98-1.87 (m, 3H), 1.50-1.40 (m, 12H), 1.05 (s, 9H).

tert-butyl 4-(7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoyl)piperazine-1-carboxylate (10.2): ¹H NMR (600 MHz, CDCl₃) δ 8.67 (s, 1H), 7.46 (d, J=7.8 Hz, 1H), 7.40 (d, 2H), 7.36 (d, 2H), 6.29 (d, J=8.8 Hz, 1H), 5.08 (p, J=7.1 Hz, 1H), 4.72 (t, J=8.0 Hz, 1H), 4.57 (d, J=8.8 Hz, 1H), 4.49 (br, 1H), 4.07 (d, J=11.6, 1.8 Hz, 1H), 3.72 (br, 1H), 3.59 (dd, J=11.3, 3.7 Hz, 1H), 3.56-3.50 (m, 2H), 3.47 (s, 1H), 3.44-3.33 (m, 6H), 2.52 (s, 3H), 2.51-2.45 (m, 1H), 2.37-2.12 (m, 4H), 2.10-2.03 (m, 1H), 2.00 (br, 3H), 1.64-1.56 (m, 2H), 1.50-1.41 (m, 12H), 1.03 (s, 9H).

tert-butyl 4-(8-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-8-oxooctanoyl)piperazine-1-carboxylate (10.3): ¹H NMR (600 MHz, CDCl₃) δ 8.67 (s, 1H), 7.48 (d, J=7.9 Hz, 1H), 7.40 (d, 2H), 7.37 (d, 2H), 6.20 (d, J=8.8 Hz, 1H), 5.08 (p, J=7.1 Hz, 1H), 4.72 (t, J=8.0 Hz, 1H), 4.57 (d, J=8.8 Hz, 1H), 4.50 (br, 1H), 4.10 (d, J=11.5, 1.8 Hz, 1H), 3.64-3.51 (m, 3H), 3.51-3.45 (m, 1H), 3.43 (br, 4H), 3.40-3.32 (m, 2H), 2.52 (s, 3H), 2.52-2.46 (m, 1H), 2.34-2.27 (m, 2H), 2.26-2.14 (m, 2H), 2.11-2.03 (m, 1H), 1.85 (br, 4H), 1.66-1.54 (m, 4H), 1.51-1.43 (m, 12H), 1.04 (s, 9H).

tert-butyl 4-(9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoyl)piperazine-1-carboxylate (10.4): ¹H NMR (600 MHz, CDCl₃) δ 8.66 (s, 1H), 7.51 (d, J=7.9 Hz, 1H), 7.39 (d, J=8.3 Hz, 2H), 7.36 (d, J=8.3 Hz, 2H), 6.20 (d, J=8.8 Hz, 1H), 5.08 (p, J=7.1 Hz, 1H), 4.71 (t, J=8.0 Hz, 1H), 4.58 (d, J=8.9 Hz, 1H), 4.49 (br, 1H), 4.07 (d, J=11.4, 1.9 Hz, 1H), 3.69 (br, 1H), 3.59 (dd, J=11.3, 3.6 Hz, 1H), 3.57-3.50 (m, 2H), 3.46 (br, 2H), 3.42 (br, 4H), 3.39-3.33 (m, 2H), 2.52 (s, 3H), 2.49-2.41 (m, 1H), 2.35-2.25 (m, 2H), 2.25-2.11 (m, 2H), 2.11-2.00 (m, 3H), 1.67-1.50 (m, 5H), 1.50-1.40 (m, 12H), 1.03 (s, 9H).

General procedure for the preparation of degraders #63-66: To a stirring solution of crude amine salt (1.0 equiv.) and acid 1.32 (1.1 equiv.) in DCM was added DIPEA (5 equiv.) at room temperature. To the mixture HATU (1.2 equiv.) was added and the reaction were stirred for 8 h at the same temperature. Solvent was removed under reduced pressure and the crude product was purified by flash column chromatography (DCM:MeOH:TEA=96:4:1). The product from column was mixed with 15 mL DCM and washed with saturated aqueous NH₄Cl solution. The organic portion was dried over Na₂SO₄, filtered, and concentrated under reduced pressure to afford the corresponding degraders.

(2S,4R)-1-((2S)-2-(5-(4-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carbonyl)piperazin-1-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide (degrader #63): ¹H NMR (600 MHz, CDCl₃) δ 8.66 (s, 1H), 8.31 (d, J=2.2 Hz, 1H), 8.07 (d, 1H), 7.70 (br, 2H), 7.50 (br, 1H), 7.41-7.31 (m, 6H), 7.25 (s, 4H), 7.02-6.92 (m, 3H), 6.88 (br, 1H), 6.72 (d, 2H), 6.58 (d, J=9.3 Hz, 1H), 5.07 (p, J=7.1 Hz, 1H), 4.72-4.64 (m, 1H), 4.56-4.48 (m, 1H), 4.46 (br, 1H), 4.07 (d, 1H), 3.87 (br, 1H), 3.74-3.59 (m, 10H), 3.56 (d, J=10.7 Hz, 2H), 3.49-3.36 (m, 3H), 3.18 (br, 4H), 3.08 (dd, J=13.8, 4.9 Hz, 1H), 3.00 (dd, J=13.8, 7.2 Hz, 1H), 2.90-2.78 (m, 3H), 2.48 (d, J=3.1 Hz, 3H), 2.46-2.22 (m, 15H), 2.22-2.14 (m, 1H), 2.14-2.05 (m, 3H), 1.89-1.77 (m, 2H), 1.76-1.69 (m, 1H), 1.69-1.61 (m, 1H), 1.46 (d, J=7.0, 1.7 Hz, 3H), 1.33 (s, 3H), 1.03 (s, 9H).

(2S,4R)-1-((2S)-2-(7-(4-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carbonyl)piperazin-1-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (degrader #64): ¹H NMR (600 MHz, CDCl₃) δ 8.66 (s, 1H), 8.29 (t, J=3.9, 2.3 Hz, 1H), 8.07 (d, J=9.2 Hz, 1H), 7.73 (d, J=8.4 Hz, 2H), 7.46-7.32 (m, 7H), 7.32-7.19 (m, 4H), 7.04-6.90 (m, 3H), 6.72 (d, 2H), 6.58 (d, J=9.3 Hz, 1H), 6.46 (dd, J=15.7, 8.6 Hz, 1H), 5.08 (p, J=7.1 Hz, 1H), 4.71-4.64 (m, 1H), 4.62-4.53 (m, 1H), 4.47 (br, 1H), 4.09 (d, J=11.3 Hz, 1H), 3.87 (br, 1H), 3.78-3.59 (m, 9H), 3.59-3.50 (m, 1H), 3.50-3.38 (m, 1H), 3.17 (br, 4H), 3.09 (dd, J=13.8, 5.0 Hz, 1H), 3.00 (dd, J=13.9, 7.2 Hz, 1H), 2.84 (dt, J=32.1, 15.0 Hz, 3H), 2.54-2.45 (m, 5H), 2.45-2.21 (m, 12H), 2.21-2.03 (m, 4H), 1.75-1.60 (m, 3H), 1.58-1.41 (m, 9H), 1.38-1.30 (m, 5H), 1.29-1.11 (m, 2H), 1.03 (d, J=4.5 Hz, 9H).

(2S,4R)-1-((2S)-2-(8-(4-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carbonyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #65): ¹H NMR (600 MHz, CDCl₃) δ 8.66 (s, 1H), 8.31 (d, J=2.2 Hz, 1H), 8.08 (d, J=9.2 Hz, 1H), 7.71 (d, J=8.5 Hz, 2H), 7.41-7.31 (m, 7H), 7.32-7.19 (m, 5H), 7.04-6.92 (m, 3H), 6.74 (d, J=8.5 Hz, 2H), 6.59 (d, J=9.3 Hz, 1H), 6.42 (dd, J=26.5, 8.9 Hz, 1H), 5.08 (h, J=6.9 Hz, 1H), 4.72-4.62 (m, 1H), 4.62-4.54 (m, 1H), 4.47 (br, 1H), 4.09 (d, J=11.4 Hz, 1H), 3.88 (br, 1H), 3.77-3.51 (m, 11H), 3.51-3.38 (m, 2H), 3.19 (br, 4H), 3.09 (dd, J=13.9, 5.0 Hz, 1H), 3.00 (dd, J=13.9, 7.2 Hz, 1H), 2.90-2.75 (m, 3H), 2.50 (s, 3H), 2.47-2.22 (m, 12H), 2.22-2.00 (m, 4H), 1.78-1.69 (m, 1H), 1.69-1.61 (m, 1H), 1.52 (br, 4H), 1.46 (t, J=6.7 Hz, 3H), 1.35 (s, 3H), 1.31-1.12 (m, 9H), 1.03 (s, 9H).

(2S,4R)-1-((2S)-2-(9-(4-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carbonyl)piperazin-1-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide (degrader #65): ¹H NMR (600 MHz, CDCl₃) δ 8.68 (s, 1H), 8.33 (d, J=2.2 Hz, 1H), 8.10 (d, J=9.1 Hz, 1H), 7.74 (d, J=8.4 Hz, 2H), 7.50-7.34 (m, 7H), 7.34-7.21 (m, 5H), 7.06-6.94 (m, 3H), 6.81-6.71 (m, 2H), 6.61 (d, J=9.3 Hz, 1H), 6.45 (dd, J=14.1, 8.9 Hz, 1H), 5.10 (td, J=7.3, 2.9 Hz, 1H), 4.69 (t, J=8.2 Hz, 1H), 4.61 (dd, J=9.0, 4.3 Hz, 1H), 4.49 (br, 1H), 4.10 (d, J=11.5 Hz, 1H), 3.90 (br, 1H), 3.79-3.62 (m, 11H), 3.59 (d, J=11.1 Hz, 2H), 3.54-3.42 (m, 3H), 3.23 (br, 4H), 3.11 (dd, J=13.8, 5.0 Hz, 1H), 3.02 (dd, J=13.8, 7.2 Hz, 1H), 2.93-2.80 (m, 3H), 2.52 (s, 3H), 2.49-2.25 (m, 12H), 2.25-2.06 (m, 4H), 1.81-1.72 (m, 1H), 1.72-1.63 (m, 1H), 1.56 (br, 6H), 1.48 (dd, J=6.9, 3.6 Hz, 3H), 1.37 (s, 3H), 1.25 (br, 8H), 1.05 (s, 9H).

### Example 44: Preparation of Degraders #67-69

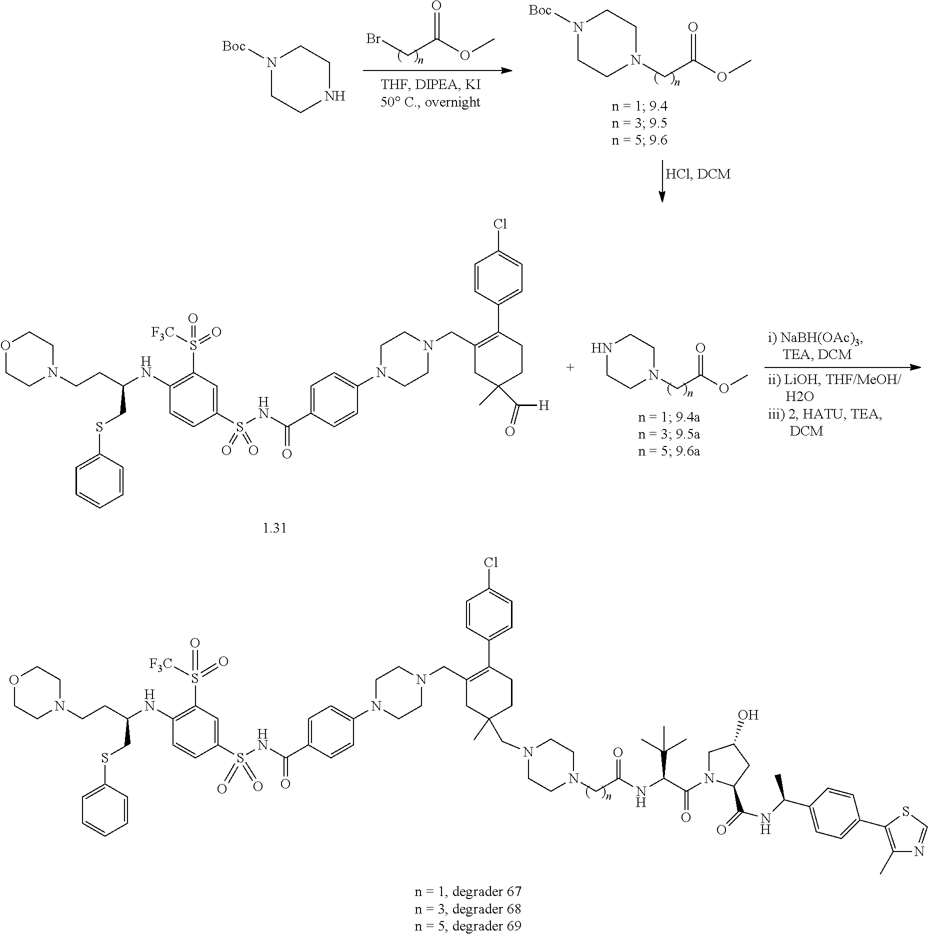

Degraders #67-69 were prepared by following the same synthetic protocol as that of degrader #52.

tert-butyl 4-(2-methoxy-2-oxoethyl)piperazine-1-carboxylate (9.4): ¹H NMR (600 MHz, Chloroform-d) δ 3.73 (s, 3H), 3.48 (t, J=5.0 Hz, 4H), 3.24 (s, 2H), 2.52 (t, J=5.1 Hz, 4H), 1.46 (s, 9H).

tert-butyl 4-(4-methoxy-4-oxobutyl)piperazine-1-carboxylate (9.5): ¹H NMR (600 MHz, Chloroform-d) δ 3.67 (s, 3H), 3.40 (t, J=5.1 Hz, 4H), 2.40-2.30 (m, 8H), 1.81 (p, J=7.3 Hz, 2H), 1.45 (s, 9H).

tert-butyl 4-(6-methoxy-6-oxohexyl)piperazine-1-carboxylate (9.6): ¹H NMR (600 MHz, Chloroform-d) δ 3.66 (s, 3H), 3.42 (t, J=5.1 Hz, 4H), 2.36 (t, J=4.9 Hz, 4H), 2.32 (q, 4H), 1.68-1.59 (m, 2H), 1.53-1.47 (m, 2H), 1.45 (s, 9H), 1.37-1.29 (m, 2H).

(2S,4R)-1-((2S)-2-(2-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)acetamido)-3,3-dimethylbutanoyl)-4-hydroxy-N—((S)-1-(4-(4-methylthiazol-5-yl)phenyl)ethyl)pyrrolidine-2-carboxamide (degrader #67): ¹H NMR (600 MHz, CDCl₃) δ 8.67 (d, J=1.6 Hz, 1H), 8.30 (d, J=2.3 Hz, 1H), 8.10 (dd, J=9.3, 2.3 Hz, 1H), 7.67 (t, J=9.2 Hz, 2H), 7.41-7.34 (m, 7H), 7.31-7.27 (m, 2H), 7.26-7.22 (m, 3H), 7.02 (d, J=8.6 Hz, 1H), 6.99-6.94 (m, 2H), 6.75 (dd, J=9.1, 6.7 Hz, 2H), 6.59 (d, J=9.1 Hz, 1H), 5.07 (p, J=7.1 Hz, 1H), 4.66 (q, J=8.0 Hz, 1H), 4.47 (d, J=10.1 Hz, 2H), 4.06 (d, J=13.6 Hz, 1H), 3.88 (q, J=7.0, 5.9 Hz, 1H), 3.64 (td, J=6.3, 3.5 Hz, 4H), 3.57 (dd, J=11.4, 3.5 Hz, 1H), 3.27-3.21 (m, 4H), 3.09 (dd, J=13.9, 5.0 Hz, 1H), 3.03-2.97 (m, 3H), 2.81 (s, 2H), 2.62 (s, 4H), 2.54 (s, 4H), 2.48 (s, 3H), 2.44-2.20 (m, 16H), 2.14-2.05 (m, 3H), 1.90 (s, 1H), 1.69-1.65 (m, 1H), 1.60-1.54 (m, 1H), 1.47 (d, J=7.8 Hz, 3H), 1.45-1.40 (m, 1H), 1.03 (d, J=2.8 Hz, 9H), 0.92 (d, J=4.3 Hz, 3H).

(2S,4R)-1-((2S)-2-(4-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)butanamido)-3,3-dimethylbutanoyl)-4-hydroxy-N—((S)-1-(4-(4-methylthiazol-5-yl)phenyl)ethyl)pyrrolidine-2-carboxamide (degrader #68): ¹H NMR (600 MHz, CDCl₃) δ 8.65 (d, J=6.9 Hz, 2H), 8.30 (t, J=2.8 Hz, 1H), 7.95 (t, J=11.4 Hz, 1H), 7.89-7.84 (m, 2H), 7.54 (t, J=8.2 Hz, 1H), 7.40-7.29 (m, 9H), 7.24-7.18 (m, 2H), 6.97 (dd, J=8.4, 3.2 Hz, 2H), 6.82 (d, J=8.6 Hz, 1H), 6.73 (t, J=7.8 Hz, 2H), 6.51 (dd, J=9.5, 3.4 Hz, 1H), 5.06 (p, J=7.3 Hz, 2H), 4.70 (dq, J=17.0, 8.2 Hz, 2H), 4.57-4.41 (m, 3H), 4.03 (d, J=11.2 Hz, 1H), 3.86-3.73 (m, 2H), 3.68-3.50 (m, 7H), 3.27-3.04 (m, 7H), 2.95 (dd, Hz, 1H), 3.86-3.73 (m, 2H), 3.68-3.50 (m, 7H), 3.27-3.04 (m, 7H), 2.95 (dd, J=13.7, 7.4 Hz, 2H), 2.89-2.58 (m, 12H), 2.50 (d, J=8.1 Hz, 5H), 2.35-2.16 (m, 17H), 1.03-0.99 (m, 12H).

(2S,4R)-1-((2S)-2-(6-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)hexanamido)-3,3-dimethylbutanoyl)-4-hydroxy-N—((S)-1-(4-(4-methylthiazol-5-yl)phenyl)ethyl)pyrrolidine-2-carboxamide (degrader #69): ¹H NMR (600 MHz, CDCl₃) δ 8.64 (s, 1H), 8.29 (s, 1H), 7.94 (d, J=8.9 Hz, 1H), 7.85 (d, J=8.3 Hz, 2H), 7.40-7.29 (m, 8H), 7.25-7.18 (m, 4H), 6.98 (d, J=7.9 Hz, 2H), 6.82 (d, J=8.5 Hz, 1H), 6.73 (d, J=8.8 Hz, 2H), 6.54 (d, J=9.3 Hz, 1H), 5.04 (p, J=6.6 Hz, 1H), 4.67 (d, J=15.6 Hz, 1H), 4.44 (s, 2H), 4.04 (s, 1H), 3.80 (d, J=20.1 Hz, 1H), 3.64-3.55 (m, 5H), 3.13 (s, 4H), 3.09-3.05 (m, 1H), 2.99-2.95 (m, 1H), 2.79 (s, 8H), 2.49 (d, J=12.1 Hz, 4H), 2.41-2.12 (m, 21H), 1.83 (s, 7H), 1.68-1.42 (m, 13H), 0.99 (d, J=9.0 Hz, 12H), 0.93-0.88 (m, 3H).

### Example 45: Preparation of Degraders #70-75

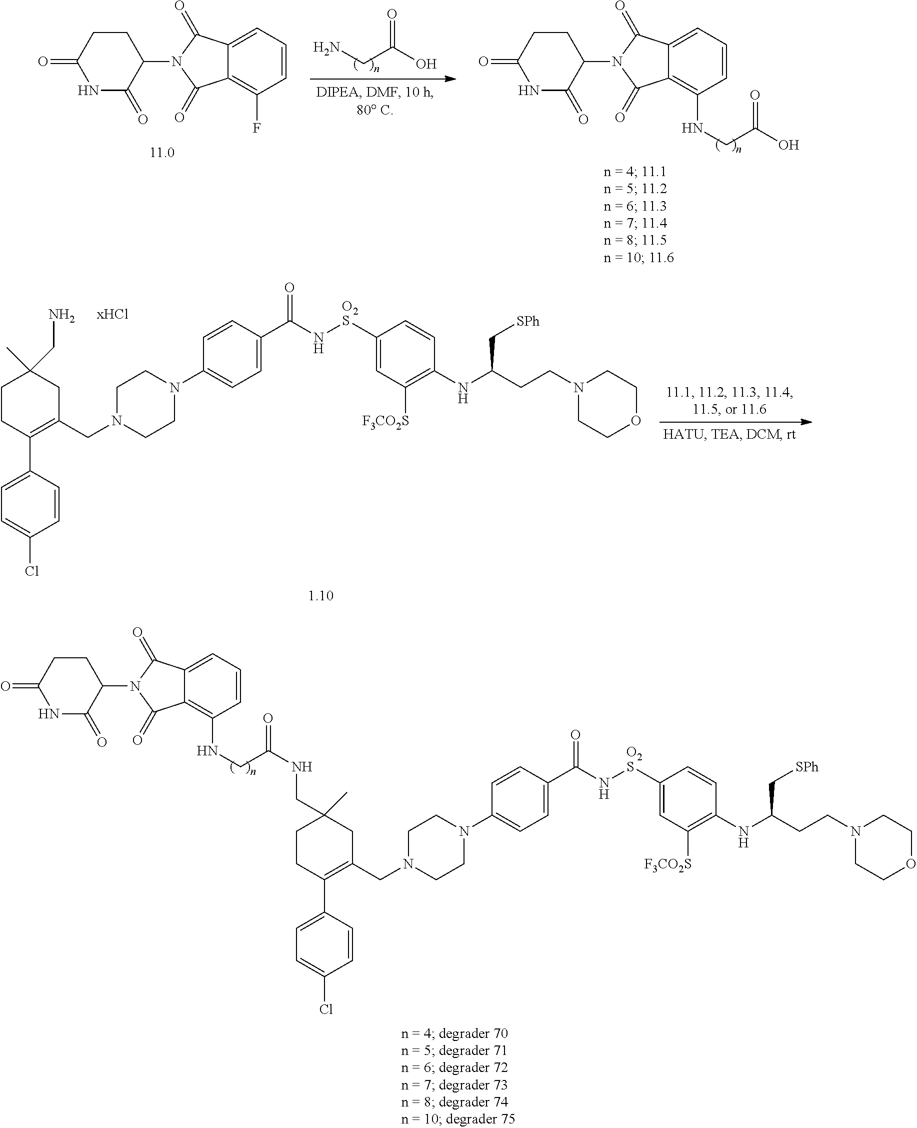

General procedure for the preparation of acids 11.1-11.6: To a stirring solution of compound 11.0 (1.0 equiv.) and a suitable amino acid (1.5 equiv.) in DMF was added DIPEA (2.0 equiv.) and the mixture was stirred for 10 h at 80° C. DMF was removed under reduced pressure and the crude product was purified by silica gel flash chromatography.

5-((2-(2,6-dioxopiperidin-3-yl)-1,3-dioxoisoindolin-4-yl)amino)pentanoic acid (11.1): ¹H NMR (600 MHz, CDCl₃) δ 8.67 (s, 1H), 7.48 (dd, J=8.6, 7.1 Hz, 1H), 7.09 (d, J=7.1 Hz, 1H), 6.87 (d, J=8.5 Hz, 1H), 6.26 (t, J=5.7 Hz, 1H), 4.94-4.89 (m, 1H), 3.33-3.25 (m, 2H), 2.90-2.83 (m, 1H), 2.82-2.70 (m, 2H), 2.41 (t, J=6.8 Hz, 2H), 2.15-2.07 (m, 1H), 1.74 (dddt, J=16.5, 9.8, 7.0, 3.5 Hz, 4H).

6-((2-(2,6-dioxopiperidin-3-yl)-1,3-dioxoisoindolin-4-yl)amino)hexanoic acid (11.2): ¹H NMR (600 MHz, CDCl₃) δ 8.79 (s, 1H), 7.51-7.42 (m, 1H), 7.07 (d, J=7.0 Hz, 1H), 6.86 (d, J=8.5 Hz, 1H), 6.23 (s, 1H), 4.92 (dd, J=12.3, 5.4 Hz, 1H), 3.27 (q, J=6.2 Hz, 2H), 2.89-2.83 (m, 1H), 2.82-2.69 (m, 2H), 2.35 (t, J 15=7.3 Hz, 2H), 2.14-2.08 (m, 1H), 1.68 (q, J=7.4 Hz, 4H), 1.46 (p, J=7.8 Hz, 2H).

7-((2-(2,6-dioxopiperidin-3-yl)-1,3-dioxoisoindolin-4-yl)amino)heptanoic acid (11.3): ¹H NMR (600 MHz, CDCl₃) δ 8.44 (s, 1H), 7.48 (dd, J=8.5, 7.1 Hz, 1H), 7.08 (d, J=7.0 Hz, 1H), 6.87 (d, J=8.5 Hz, 1H), 6.23 (t, J=5.7 Hz, 1H), 4.92 (dd, J=12.4, 5.3 Hz, 1H), 3.98 (p, J=5.0 Hz, 1H), 3.26 (q, J=6.7 Hz, 2H), 2.88 (dd, J=16.5, 3.5 Hz, 1H), 2.83-2.70 (m, 2H), 2.36 (t, J=7.4 Hz, 2H), 2.13 (dtd, J=10.3, 5.2, 3.0 Hz, 1H), 1.66 (t, J=6.9 Hz, 4H), 1.42 (dd, J=13.8, 8.9 Hz, 3H).

8-((2-(2,6-dioxopiperidin-3-yl)-1,3-dioxoisoindolin-4-yl)amino)octanoic acid (11.4): ¹H NMR (600 MHz, CDCl₃) δ 8.22 (s, 1H), 7.49 (dd, J=8.6, 7.1 Hz, 1H), 7.09 (d, J=6.9 Hz, 1H), 6.88 (d, J=8.5 Hz, 1H), 6.23 (t, J=5.6 Hz, 1H), 4.95-4.86 (m, 1H), 3.26 (q, J=6.8 Hz, 2H), 2.93-2.86 (m, 1H), 2.84-2.69 (m, 2H), 2.35 (t, J=7.4 Hz, 2H), 2.13 (dtd, J=10.2, 5.2, 3.0 Hz, 1H), 1.70-1.60 (m, 4H), 1.46-1.40 (m, 2H), 1.37 (dt, J=7.4, 3.7 Hz, 4H).

9-((2-(2,6-dioxopiperidin-3-yl)-1,3-dioxoisoindolin-4-yl)amino)nonanoic acid (11.5): ¹H NMR (600 MHz, CDCl₃) δ 8.46 (s, 1H), 7.48 (dd, J=8.5, 7.1 Hz, 1H), 7.08 (d, J=7.1 Hz, 1H), 6.87 (d, J=8.5 Hz, 1H), 6.23 (t, J=5.6 Hz, 1H), 4.92 (dd, J=12.3, 5.4 Hz, 1H), 3.26 (q, J=6.5 Hz, 2H), 2.92-2.85 (m, 1H), 2.84-2.69 (m, 2H), 2.34 (t, J=7.4 Hz, 2H), 2.13 (ddd, J=12.6, 6.3, 4.1 Hz, 1H), 1.65 (dq, J=15.5, 8.1, 7.5 Hz, 4H), 1.45-1.39 (m, 2H), 1.34 (s, 6H).

11-((2-(2,6-dioxopiperidin-3-yl)-1,3-dioxoisoindolin-4-yl)amino)undecanoic acid (11.6): ¹H NMR (600 MHz, CDCl₃) δ 8.48 (s, 1H), 7.48 (dd, J=8.5, 7.1 Hz, 1H), 7.08 (d, J=7.1 Hz, 1H), 6.88 (d, J=8.5 Hz, 1H), 6.23 (t, J=5.6 Hz, 1H), 4.92 (dd, J=12.4, 5.4 Hz, 1H), 3.25 (q, J=6.7 Hz, 2H), 2.88 (dd, J=16.6, 3.5 Hz, 1H), 2.84-2.70 (m, 2H), 2.34 (t, J=7.4 Hz, 2H), 2.13 (ddd, J=12.7, 6.3, 4.1 Hz, 1H), 1.68-1.60 (m, 4H), 1.40 (q, J=7.3 Hz, 2H), 1.30 (d, J=15.1 Hz, 12H).

General procedure for the preparation of degraders #70-75: To a stirring solution of amine 1.10 (12 mg, 0.011 mmol) and acid 11.x (1.1 equiv.) in DCM (1.5 mL) was added TEA (0.01 ml, 0.066 mmol) at room temperature. To the mixture HATU (5 mg, 0.012 mmol) was added and the reaction were stirred for 8 h at the same temperature. Solvent was removed under reduced pressure and the crude product was purified by flash column chromatography (DCM/MeOH/TEA=96:3:1). The product from column was mixed with 15 mL DCM and washed with saturated aqueous NH₄Cl solution. The organic portion was dried over Na₂SO₄, filtered, and concentrated under reduced pressure to afford the corresponding degrader.

4-(4-((4′-chloro-4-((5-((2-(2,6-dioxopiperidin-3-yl)-1,3-dioxoisoindolin-4-yl)amino)pentanamido)methyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (degrader #70): ¹H NMR (600 MHz, CDCl₃) δ 9.01 (s, 1H), 8.35 (d, J=10.2 Hz, 1H), 8.06 (d, J=5.7 Hz, 1H), 7.67-7.47 (m, 2H), 7.39 (t, J=5.9 Hz, 4H), 7.35-7.29 (m, 3H), 7.28 (d, J=4.8 Hz, 3H), 7.03 (t, J=7.1 Hz, 2H), 6.90 (s, 2H), 6.78-6.58 (m, 1H), 6.41-6.11 (m, 1H), 4.87 (dd, J=13.2, 5.1 Hz, 1H), 4.02-3.86 (m, 1H), 3.70 (s, 4H), 3.18-2.94 (m, 5H), 2.94-2.79 (m, 4H), 2.79-2.68 (m, 3H), 2.34 (s, 11H), 2.14 (s, 3H), 1.71-1.60 (m, 9H), 1.25 (s, 6H), 1.02 (d, J=11.9 Hz, 3H).

4-(4-((4′-chloro-4-((6-((2-(2,6-dioxopiperidin-3-yl)-1,3-dioxoisoindolin-4-yl)amino)hexanamido)methyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (degrader #71): ¹H NMR (600 MHz, CDCl₃) δ 8.88 (s, 1H), 8.36 (s, 1H), 8.08-8.01 (m, 1H), 7.63 (d, J=8.5 Hz, 2H), 7.37 (dd, J=13.9, 7.7 Hz, 5H), 7.31 (t, J=7.7 Hz, 2H), 7.02 (d, J=8.3 Hz, 2H), 6.97-6.88 (m, 2H), 6.70 (s, 1H), 6.66 (d, J=9.2 Hz, 1H), 6.48-6.32 (m, 2H), 6.00-5.90 (m, 1H), 4.87 (dd, J=11.1, 5.0 Hz, 1H), 3.98-3.84 (m, 1H), 3.66 (s, 5H), 3.54 (dd, J=6.4, 4.2 Hz, 1H), 3.39 (d, J=18.2 Hz, 1H), 3.25-3.05 (m, 4H), 3.02 (dd, J=13.8, 7.2 Hz, 1H), 2.94 (dd, J=11.9, 4.5 Hz, 2H), 2.85 (d, J=10.3 Hz, 1H), 2.74 (t, J=9.9 Hz, 2H), 2.50-2.26 (m, 8H), 2.26-2.15 (m, 2H), 2.10 (dt, J=17.3, 5.1 Hz, 2H), 2.03-1.71 (m, 2H), 1.70-1.57 (m, 4H), 1.54 (d, J=5.9 Hz, 3H), 1.47-1.33 (m, 2H), 1.24 (d, J=14.8 Hz, 7H), 1.05-0.97 (m, 3H).

4-(4-((4′-chloro-4-((7-((2-(2,6-dioxopiperidin-3-yl)-1,3-dioxoisoindolin-4-yl)amino)heptanamido)methyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (degrader #72): ¹H NMR (600 MHz, CDCl₃) δ 8.85 (d, J=21.2 Hz, 1H), 8.35 (d, J=5.2 Hz, 1H), 8.04 (d, J=9.1 Hz, 1H), 7.70-7.63 (m, 2H), 7.45-7.41 (m, 1H), 7.38 (d, J=7.4 Hz, 2H), 7.35-7.28 (m, 4H), 7.02 (dd, J=7.0, 3.3 Hz, 1H), 6.99 (d, J=8.1 Hz, 2H), 6.94 (d, J=7.5 Hz, 1H), 6.80 (d, J=8.4 Hz, 1H), 6.63 (d, J=9.3 Hz, 1H), 6.54-6.44 (m, 2H), 6.06 (s, 1H), 4.90 (dt, J=11.8, 6.0 Hz, 1H), 3.91 (s, 1H), 3.67 (s, 4H), 3.56-3.44 (m, 2H), 3.23 (s, 3H), 3.14-2.94 (m, 4H), 2.90-2.82 (m, 1H), 2.74 (td, J=17.8, 9.2 Hz, 2H), 2.52-2.26 (m, 9H), 2.21 (t, J=6.6 Hz, 2H), 2.17-2.07 (m, 2H), 1.72-1.50 (m, 6H), 1.41 (q, J=8.4, 7.3 Hz, 3H), 1.33-1.17 (m, 11H), 1.00 (s, 3H).

4-(4-((4′-chloro-4-((8-((2-(2,6-dioxopiperidin-3-yl)-1,3-dioxoisoindolin-4-yl)amino)octanamido)methyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (degrader #73): ¹H NMR (600 MHz, CDCl₃) δ 8.87 (s, 1H), 8.37-8.32 (m, 1H), 8.05 (d, J=9.1 Hz, 1H), 7.67 (d, J=8.5 Hz, 2H), 7.45 (q, J=7.2 Hz, 1H), 7.38 (d, J=7.5 Hz, 2H), 7.34-7.28 (m, 4H), 7.04 (t, J=8.0 Hz, 1H), 7.00 (d, J=8.3 Hz, 2H), 6.94 (d, J=7.9 Hz, 1H), 6.82 (d, J=7.5 Hz, 1H), 6.63 (d, J=9.3 Hz, 1H), 6.54 (s, 2H), 6.09 (s, 1H), 4.91 (dd, J=10.6, 4.6 Hz, 1H), 3.90 (s, 1H), 3.66 (s, 4H), 3.51-3.37 (m, 2H), 3.25 (s, 4H), 3.11 (dd, J=13.5, 4.8 Hz, 4H), 3.02 (dd, J=13.7, 7.2 Hz, 2H), 2.86 (d, J=15.0 Hz, 2H), 2.79-2.70 (m, 3H), 2.47-2.30 (m, 11H), 2.20 (s, 3H), 2.17-2.06 (m, 4H), 1.68 (dd, J=13.8, 8.1 Hz, 2H), 1.64-1.56 (m, 3H), 1.48-1.43 (m, 2H), 1.24-1.15 (m, 6H), 1.00 (s, 3H).

4-(4-((4′-chloro-4-((9-((2-(2,6-dioxopiperidin-3-yl)-1,3-dioxoisoindolin-4-yl)amino)nonanamido)methyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (degrader #74): ¹H NMR (600 MHz, CDCl₃) δ 8.78 (s, 1H), 8.35 (d, J=1.9 Hz, 1H), 8.05 (d, J=9.0 Hz, 1H), 7.67 (d, J=7.7 Hz, 2H), 7.48-7.43 (m, 1H), 7.37 (d, J=7.5 Hz, 2H), 7.30 (dt, J=7.6, 3.3 Hz, 4H), 7.06 (d, J=7.1 Hz, 1H), 6.99 (d, J=8.3 Hz, 2H), 6.95 (d, J=8.2 Hz, 1H), 6.84 (d, J=8.6 Hz, 1H), 6.61 (d, J=9.0 Hz, 1H), 6.56 (d, J=8.0 Hz, 2H), 6.16 (t, J=5.1 Hz, 1H), 4.91 (dd, J=12.3, 5.4 Hz, 1H), 3.94-3.84 (m, 1H), 3.66 (td, J=6.4, 3.5 Hz, 4H), 3.45-3.38 (m, 1H), 3.25 (s, 5H), 3.21-3.15 (m, 2H), 3.12-3.08 (m, 2H), 3.01 (dd, J=13.8, 7.3 Hz, 2H), 2.86 (d, J=15.6 Hz, 1H), 2.82-2.64 (m, 4H), 2.44 (s, 2H), 2.41-2.30 (m, 8H), 2.19 (t, J=7.0 Hz, 2H), 2.14-2.09 (m, 2H), 1.71-1.64 (m, 1H), 1.55 (dt, J=21.6, 7.6 Hz, 6H), 1.35-1.27 (m, 3H), 1.25 (s, 3H), 1.21 (s, 6H), 0.99 (s, 3H).

4-(4-((4′-chloro-4-((11-((2-(2,6-dioxopiperidin-3-yl)-1,3-dioxoisoindolin-4-yl)amino)undecanamido)methyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (degrader #75): ¹H NMR (600 MHz, CDCl₃) δ 8.65 (d, J=27.6 Hz, 1H), 8.35 (d, J=1.9 Hz, 1H), 8.07 (d, J=9.1 Hz, 1H), 7.66 (d, J=8.8 Hz, 2H), 7.50-7.45 (m, 1H), 7.38 (d, J=7.4 Hz, 2H), 7.30 (t, J=7.2 Hz, 4H), 7.07 (d, J=7.1 Hz, 1H), 7.00 (d, J=8.3 Hz, 3H), 6.87 (d, J=8.6 Hz, 1H), 6.61 (d, J=9.3 Hz, 3H), 6.19 (t, J=5.4 Hz, 1H), 4.91 (dt, J=12.4, 4.9 Hz, 1H), 3.93-3.85 (m, 1H), 3.66 (s, 4H), 3.40 (dd, J=15.3, 6.6 Hz, 1H), 3.29 (d, J=15.3 Hz, 3H), 3.22 (q, J=6.7 Hz, 3H), 3.10 (dd, J=13.8, 5.0 Hz, 1H), 3.06 (d, J=3.8 Hz, 1H), 3.02 (dd, J=13.8, 7.3 Hz, 1H), 2.89-2.82 (m, 1H), 2.82-2.67 (m, 3H), 2.67-2.51 (m, 2H), 2.50-2.42 (m, 2H), 2.42-2.23 (m, 9H), 2.20 (t, J=7.3 Hz, 3H), 2.15-2.10 (m, 2H), 1.67 (dd, J=13.2, 5.8 Hz, 2H), 1.60 (dq, J=12.7, 6.5, 5.8 Hz, 5H), 1.53 (dt, J=12.9, 7.0 Hz, 2H), 1.37-1.31 (m, 3H), 1.20 (dd, J=9.1, 4.7 Hz, 10H), 0.99 (s, 3H).

### Example 46: Preparation of Degraders #76-78

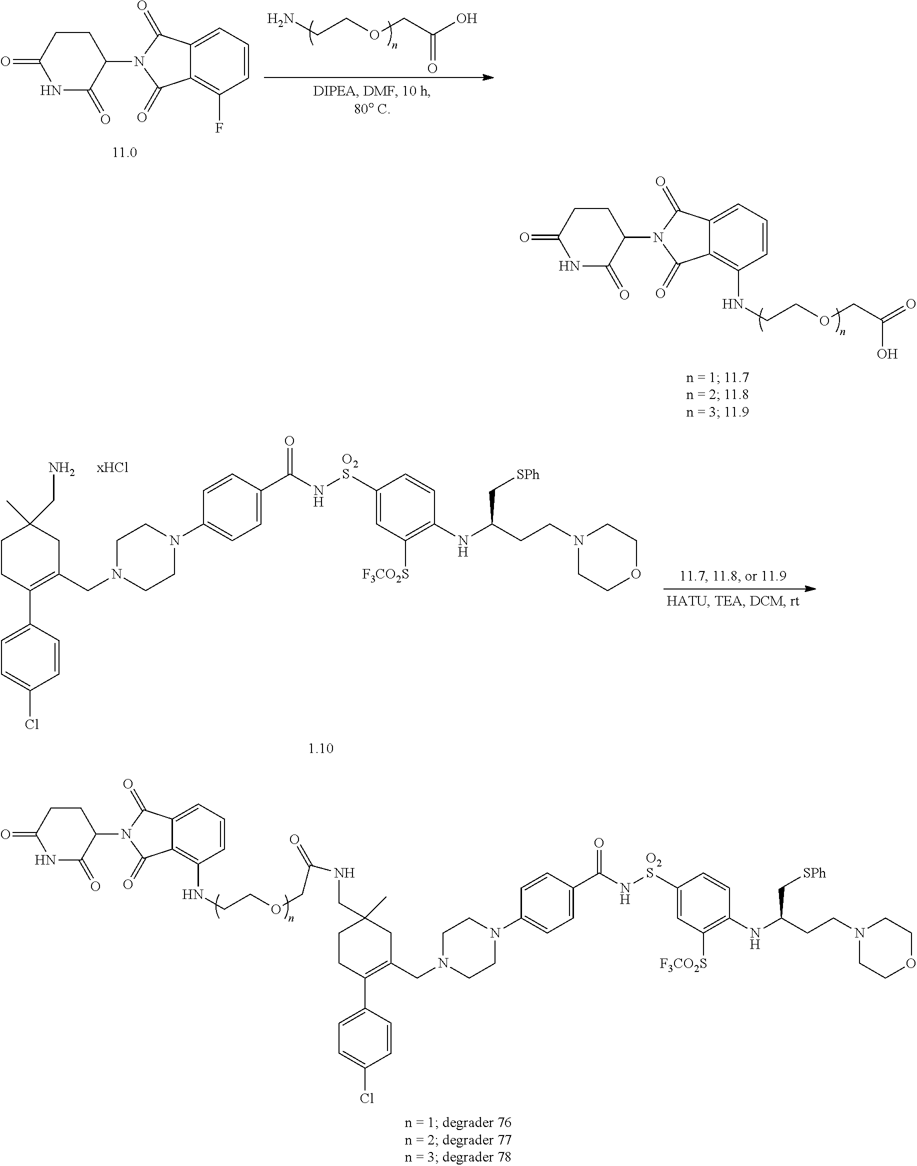

General procedure for the preparation of acids 11.7-11.9: To a stirring solution of compound 11.0 (1.0 equiv.) and a suitable amino acid (1.5 equiv.) in DMF was added DIPEA (2.0 equiv.). The mixture was stirred for 10 h at 80° C. DMF was removed under reduced pressure and the crude product was purified by silica gel flash chromatography to afford the desired acid.

2-(2-((2-(2,6-dioxopiperidin-3-yl)-1,3-dioxoisoindolin-4-yl)amino)ethoxy)acetic acid (11.7): ¹H NMR (600 MHz, CDCl₃) δ 7.58 (d, J=47.1 Hz, 1H), 7.36 (d, J=7.8 Hz, 1H), 6.95 (d, J=6.6 Hz, 1H), 6.80 (t, J=7.0 Hz, 1H), 4.86 (dd, J=13.1, 6.1 Hz, 1H), 3.95 (s, 3H), 3.81 (s, 1H), 3.69 (s, 3H), 2.76-2.61 (m, 3H), 2.11-1.96 (m, 1H).

2-(2-(2-((2-(2,6-dioxopiperidin-3-yl)-1,3-dioxoisoindolin-4-yl)amino)ethoxy)ethoxy)acetic acid (11.8): ¹H NMR (600 MHz, CDCl₃) δ 9.05 (s, 1H), 7.51-7.43 (m, 1H), 7.08 (d, J=7.1 Hz, 1H), 6.90 (d, J=8.6 Hz, 1H), 4.95 (dd, J=12.1, 5.6 Hz, 1H), 4.18 (s, 2H), 3.72 (d, J=35.3 Hz, 6H), 3.48 (t, J=5.1 Hz, 2H), 2.85 (s, 1H), 2.77 (s, 2H), 2.10 (s, 1H).

2-(2-(2-(2-((2-(2,6-dioxopiperidin-3-yl)-1,3-dioxoisoindolin-4-yl)amino)ethoxy)ethoxy)ethoxy)acetic acid (11.9): ¹H NMR (600 MHz, CDCl₃) δ 8.90 (s, 1H), 7.47 (dd, J=8.5, 7.2 Hz, 1H), 7.08 (d, J=7.0 Hz, 1H), 6.91 (d, J=8.5 Hz, 1H), 4.93 (dd, J=12.2, 5.5 Hz, 1H), 4.14 (s, 2H), 3.70 (d, J=38.3 Hz, 10H), 3.47 (t, J=5.3 Hz, 2H), 2.85 (d, J=26.5 Hz, 1H), 2.75 (d, J=46.4 Hz, 2H), 2.11 (d, J=36.7 Hz, 1H).

General procedure for the preparation of degraders #76-78: To a stirring solution of amine 1.10 (1.0 equiv.) and acid 11.7, 11.8, or 11.9 (1.1 equiv.) in DCM was added TEA (10 equiv.) at room temperature. To the mixture HATU (1.2 equiv.) was added and the reaction were stirred for 8 h at the same temperature. Solvent was removed under reduced pressure and the crude product was purified by flash column chromatography (DCM/MeOH/TEA=96:5:1). The product from column was mixed with 15 mL DCM and washed with saturated aqueous NH₄Cl solution. The organic portion was dried over Na₂SO₄, filtered, and concentrated under reduced pressure to afford the corresponding degrader.

4-(4-((4′-chloro-4-((2-(2-((2-(2,6-dioxopiperidin-3-yl)-1,3-dioxoisoindolin-4-yl)amino)ethoxy)acetamido)methyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (degrader #76): ¹H NMR (600 MHz, CDCl₃) δ 8.34 (t, J=2.5 Hz, 1H), 8.12-8.06 (m, 1H), 7.62 (d, J=8.8 Hz, 2H), 7.48 (q, J=7.1 Hz, 1H), 7.37 (d, J=7.5 Hz, 2H), 7.32-7.27 (m, 4H), 7.07 (ddd, J=21.8, 9.2, 6.3 Hz, 2H), 6.99 (dd, J=8.4, 2.0 Hz, 2H), 6.92-6.88 (m, 1H), 6.82 (s, 1H), 6.66 (t, J=9.9 Hz, 2H), 6.61 (d, J=9.3 Hz, 1H), 6.50 (dt, J=68.3, 4.7 Hz, 1H), 4.94-4.87 (m, 1H), 4.10-4.02 (m, 2H), 3.92 (s, 1H), 3.82-3.70 (m, 2H), 3.70-3.62 (m, 4H), 3.53-3.45 (m, 2H), 3.40-3.35 (m, 1H), 3.27-3.12 (m, 4H), 3.12-3.08 (m, 1H), 3.02 (dd, J=13.9, 7.2 Hz, 1H), 2.98-2.82 (m, 2H), 2.80-2.68 (m, 2H), 2.36 (ddd, J=44.3, 32.2, 18.9 Hz, 12H), 2.18-2.05 (m, 4H), 2.05-1.97 (m, 1H), 1.68 (s, 3H), 1.52 (dt, J=14.0, 6.8 Hz, 3H), 0.97 (d, J=29.6 Hz, 3H).

4-(4-((4′-chloro-4-((2-(2-(2-((2-(2,6-dioxopiperidin-3-yl)-1,3-dioxoisoindolin-4-yl)amino)ethoxy)ethoxy)acetamido)methyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (degrader #77): ¹H NMR (600 MHz, CDCl3) δ 8.34 (s, 1H), 8.10 (dd, J=9.2, 1.7 Hz, 1H), 7.64 (d, J=8.5 Hz, 2H), 7.50-7.44 (m, 1H), 7.37 (d, J=7.5 Hz, 2H), 7.30 (t, J=7.5 Hz, 2H), 7.28-7.26 (m, 2H), 7.10-7.03 (m, 2H), 6.97 (dd, J=8.3, 3.9 Hz, 3H), 6.88 (d, J=8.5 Hz, 1H), 6.68 (dd, J=9.0, 3.2 Hz, 2H), 6.60 (d, J=9.4 Hz, 1H), 6.47 (dd, J=12.1, 6.1 Hz, 1H), 4.90 (dd, J=12.2, 5.0 Hz, 1H), 4.10-3.99 (m, 2H), 3.90 (dt, J=7.7, 4.1 Hz, 1H), 3.76-3.60 (m, 10H), 3.50-3.40 (m, 2H), 3.36-3.28 (m, 1H), 3.26-3.18 (m, 4H), 3.10 (dd, J=13.9, 5.0 Hz, 1H), 3.04-2.98 (m, 1H), 2.97-2.81 (m, 3H), 2.81-2.64 (m, 2H), 2.57-2.17 (m, 14H), 2.16-2.07 (m, 3H), 2.01 (d, J=17.4 Hz, 1H), 1.68 (dt, J=14.1, 6.9 Hz, 1H), 1.51 (dq, J=19.8, 6.7 Hz, 2H), 0.99 (s, 3H).

4-(4-((4′-chloro-4-(13-((2-(2,6-dioxopiperidin-3-yl)-1,3-dioxoisoindolin-4-yl)amino)-3-oxo-5,8,11-trioxa-2-azatridecyl)-4-methyl-3,4,5,6-tetrahydro-[1,1′-biphenyl]-2-yl)methyl)piperazin-1-yl)-N-((4-(((R)-4-morpholino-1-(phenylthio)butan-2-yl)amino)-3-((trifluoromethyl)sulfonyl)phenyl)sulfonyl)benzamide (degrader #78): ¹H NMR (600 MHz, CDCl₃) δ 8.35 (s, 1H), 8.11 (d, J=9.2 Hz, 1H), 7.63 (d, J=8.9 Hz, 2H), 7.50-7.46 (m, 1H), 7.37 (d, J=7.6 Hz, 2H), 7.33-7.29 (m, 2H), 7.27 (s, 2H), 7.07 (dd, J=12.2, 7.9 Hz, 2H), 6.99 (d, J=8.3 Hz, 3H), 6.90 (dd, J=8.5, 2.9 Hz, 1H), 6.70 (d, J=7.9 Hz, 2H), 6.61 (d, J=9.4 Hz, 1H), 6.47 (t, J=5.5 Hz, 1H), 4.93 (td, J=13.9, 13.1, 5.2 Hz, 1H), 4.05 (d, J=7.4 Hz, 2H), 3.93-3.87 (m, 1H), 3.68 (dd, J=17.1, 7.1 Hz, 15H), 3.48-3.42 (m, 2H), 3.32-3.18 (m, 6H), 3.10 (dd, J=13.9, 5.0 Hz, 1H), 3.02 (dd, J=13.9, 7.2 Hz, 1H), 2.85 (d, J=23.6 Hz, 2H), 2.75 (d, J=9.0 Hz, 2H), 2.43 (s, 2H), 2.34 (d, J=7.9 Hz, 10H), 2.17-2.09 (m, 3H), 2.03-1.96 (m, 2H), 1.59-1.53 (m, 2H), 1.49 (dt, J=12.7, 6.1 Hz, 2H), 1.04-0.97 (m, 3H).

### Example 47: Preparation of Degraders #79-85

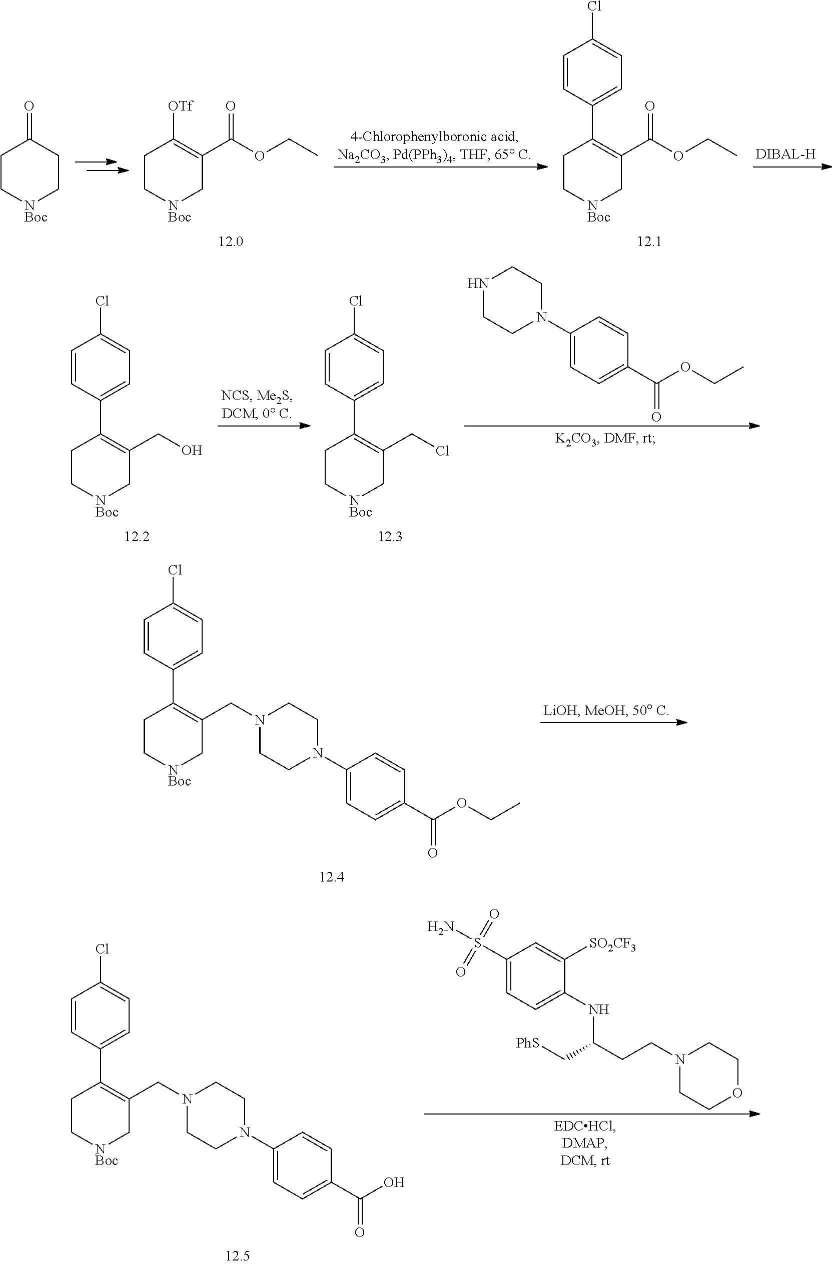

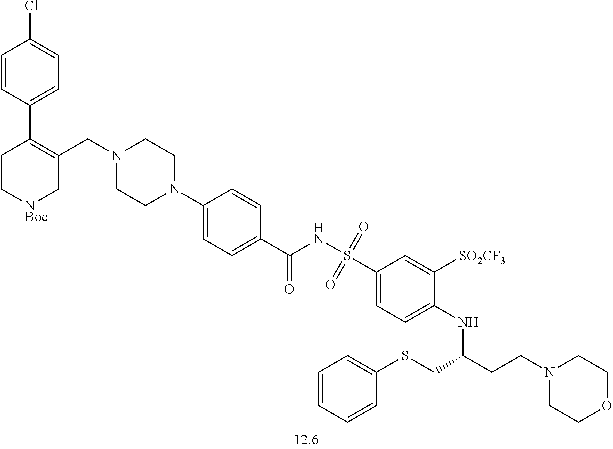

Preparation of 1-(tert-butyl) 3-ethyl 4-(((trifluoromethyl)sulfonyl)oxy)-5,6-dihydropyridine-1,3(2H)-dicarboxylate (12.0): Compound 12.0 was prepared from tert-butyl 4-oxopiperidine-1-carboxylate by following the same synthetic procedure as compound 1.3 was prepared from compound 1.1. ¹H NMR (600 MHz, CDCl₃) δ 4.36-4.21 (m, 4H), 3.61 (t, J=5.5 Hz, 2H), 2.53-2.45 (m, 2H), 1.47 (s, 9H), 1.32 (t, J=7.1 Hz, 3H).

Preparation of 1-(tert-butyl) 3-ethyl 4-(4-chlorophenyl)-5,6-dihydropyridine-1,3(2H)-dicarboxylate (12.1): To a solution of triflate 12.0 (200 mg, 0.5 mmol) and 4-chlorophenylboronic acid (93 mg, 0.6 mmol) in THF (3.4 mL) was added aqueous Na₂CO₃ solution (2.0 M, 0.77 mL). The resulting mixture was purged with N2 gas. Pd(PPh₃)₄ (10 mg, 0.0087 mmol) was then added and the mixture was stirred at 65° C. for 3 h. The mixture was filtered through a celite pad. The filtrate was diluted with ethyl acetate and washed with water and followed by brine. The organic layer was collected, dried over sodium sulfate, filtered, and condensed under reduced pressure to afford compound the title compound (150 mg, 83% yield). ¹H NMR (600 MHz, CDCl₃) δ 7.30 (d, J=8.5 Hz, 2H), 7.06 (d, J=8.5 Hz, 2H), 4.24 (s, 2H), 3.96 (q, J=7.1 Hz, 2H), 3.60 (t, J=5.6 Hz, 2H), 2.46 (s, 2H), 1.50 (s, 9H), 0.97 (s, 3H).

Preparation of tert-butyl 4-(4-chlorophenyl)-5-(hydroxymethyl)-3,6-dihydropyridine-1(2H)-carboxylate (12.2): To a solution of ester 12.1 (80 mg, 0.22 mmol) in THF (2 mL) at −78° C. was added DIBAL-H solution (1.2 M in tolulene, 0.73 mL, 0.88 mmol). The resulting mixture was stirred at −78° C. for 2-3 h until TLC showed completed consumption of the ester compound. Several drops of methanol were added to quench the reaction. After warming to room temperature, the mixture was diluted with ethyl acetate and poured into 10 mL saturated aqueous Rochelle salt solution. After stirring at room temperature overnight, the mixture was well layered. The organic phase was collected and washed with water and followed by brine, dried over sodium sulfate, filtered, and concentrated to afford a residue which was column purified (ethyl acetate/hexanes 5:1-3:1) to yield alcohol 12.2 (60 mg, 86%). ¹H NMR (600 MHz, CDCl₃) δ 7.33-7.28 (m, 2H), 7.16-7.07 (m, 2H), 4.11 (s, 2H), 4.00 (s, 2H), 3.58 (t, J=5.7 Hz, 2H), 2.37 (s, 2H), 1.48 (s, 9H).

Preparation of tert-butyl 5-(chloromethyl)-4-(4-chlorophenyl)-3,6-dihydropyridine-1(2H)-carboxylate (12.3): To a stirring solution of NCS (83 mg, 0.62 mmol) in dry DCM (1 mL) was added Me₂S (50 μL, 0.68 mmol) at 0° C. Alcohol 12.2 (100 mg, 0.31 mmol) dissolved in DCM (0.5 mL) was then added dropwise. The resulting mixture was stirred at 0° C. until full consumption of alcohol compound (approximately 1 h). Water was added to quench the reaction, and the mixture was then extracted with ethyl acetate for three times. The combined organic phases were washed with brine, dried over sodium sulfate, filtered, and condensed under reduced pressure to afford a residue which was chromatographed on silica gel (hexanes/ethyl acetate 4:1) to yield the chloride product 12.3 (100 mg, 95% yield). ¹H NMR (600 MHz, CDCl₃) δ 7.37-7.33 (m, 2H), 7.22-7.13 (m, 2H), 4.11 (s, 2H), 3.93 (s, 2H), 3.60 (t, J=5.6 Hz, 2H), 2.41 (s, 2H), 1.50 (s, 9H).

Preparation of tert-butyl 4-(4-chlorophenyl)-5-((4-(4-(ethoxycarbonyl)phenyl)piperazin-1-yl)methyl)-3,6-dihydropyridine-1(2H)-carboxylate (12.4): To a stirring solution of chloride 12.3 (50 mg, 0.15 mmol) in DMF was added ethyl 4-(piperazin-1-yl)benzoate (34.4 mg, 0.15 mmol) and Cs₂CO₃ (95 mg, 0.29 mmol). After stirring at room temperature for 1.5 h, water was added, and the mixture was extracted with ethyl acetate for three times. The combined organic phases were washed with water and brine, dried over sodium sulfate, filtered, and condensed under reduced pressure to afford a residue which was chromatographed on silica gel (hexanes/ethyl acetate 5:1) to yield ester compound 12.4 (40 mg, 51% yield). ¹H NMR (600 MHz, CDCl₃) δ 7.90 (d, J=8.9 Hz, 2H), 7.30 (d, J=8.3 Hz, 2H), 7.03 (d, J=8.3 Hz, 2H), 6.81 (d, J=8.8 Hz, 2H), 4.32 (q, J=7.1 Hz, 2H), 4.07 (s, 2H), 3.59 (t, J=5.2 Hz, 2H), 3.32-3.22 (m, 4H), 2.90 (s, 2H), 2.38 (dd, J=11.3, 6.4 Hz, 6H), 1.50 (s, 9H), 1.36 (t, J=7.1 Hz, 3H); ESI⁺, m/z[M+H]⁺=534.2.

Preparation of 4-(4-((1-(tert-butoxycarbonyl)-4-(4-chlorophenyl)-1,2,5,6-tetrahydropyridin-3-yl)methyl)piperazin-1-yl)benzoic acid (12.5): To a stirring solution of 12.4 (200 mg, 0.37 mmol) in methanol (3 mL) was added aqueous LiOH (2 N, 1 mL). The resulting mixture was heated to 55° C. and stirred at this temperature for 3 h. Upon cool down to room temperature, the pH of the mixture was adjusted to 7.0 with 3 N aqueous HCl solution. The mixture was then extracted with ethyl acetate (×3) and the combined organic layers were washed with brine, dried over sodium sulphate, and condensed to afford a residue which was chromatographed on silica gel (hexanes/ethyl acetate 3:1) to afford product 12.5 (180 mg, 95% yield). ¹H NMR (600 MHz, CDCl₃) δ 7.95 (d, J=8.7 Hz, 2H), 7.33 (d, J=8.1 Hz, 2H), 7.07-7.00 (m, 2H), 6.81 (d, J=8.6 Hz, 2H), 4.11 (s, 2H), 3.60 (t, J=5.4 Hz, 2H), 3.35 (s, 4H), 3.07 (s, 2H), 2.8-2.15 (m, 6H), 1.49 (s, 9H); ESI⁺, m/z [M+H]⁺=512.2.

Preparation of tert-butyl (R)-4-(4-chlorophenyl)-5-((4-(4-(((4-((4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridine-1(2H)-carboxylate (12.6): To a stirring solution of 12.5 (100 mg, 0.2 mmol) in DCM (2.5 mL) was added (R)-4-((4-morpholino-1-(phenylthio)butan-2-yl)amino)-3-((trifluoromethyl)sulfonyl)benzenesulfonamide (97 mg, 0.18 mmol), DMAP (48 mg, 0.39 mmol), and N-(3-Dimethylaminopropyl)-N′-ethylcarbodiimide hydrochloride (41 mg, 0.22 mmol) separately. The resulting mixture was allowed to stir at room temperature overnight and then condensed under reduced pressure to afford a residue which was chromatographed on silica gel (20:1 DCM:MeOH) to yield 12.6 as a yellowish solid (127 mg, 62% yield). 1H NMR (600 MHz, CDCl₃) δ 8.36 (d, J=2.2 Hz, 1H), 8.11 (dd, J=9.2, 2.1 Hz, 1H), 7.66 (d, J=9.0 Hz, 2H), 7.37 (dd, J=5.2, 3.4 Hz, 2H), 7.34-7.26 (m, 5H), 7.26-7.24 (m, 1H), 7.07 (d, J=8.6 Hz, 1H), 7.03-6.98 (m, 2H), 6.78 (d, J=8.8 Hz, 2H), 6.61 (d, J=9.5 Hz, 1H), 4.06 (s, 2H), 3.96-3.87 (m, 1H), 3.70-3.64 (m, 5H), 3.64-3.57 (m, 3H), 3.49-3.42 (m, 1H), 3.26 (s, 4H), 3.10 (dd, J=13.9, 5.1 Hz, 1H), 3.02 (dd, J=13.9, 7.2 Hz, 1H), 2.89 (s, 2H), 2.50-2.42 (m, 2H), 2.41-2.30 (m, 10H), 2.12 (ddd, J=10.4, 5.1, 1.9 Hz, 1H), 2.10 (s, 1H), 1.68 (dq, J=8.1, 5.6 Hz, 1H), 1.49 (s, 9H). ESI⁺, m/z [M+H]⁺=1047.2.

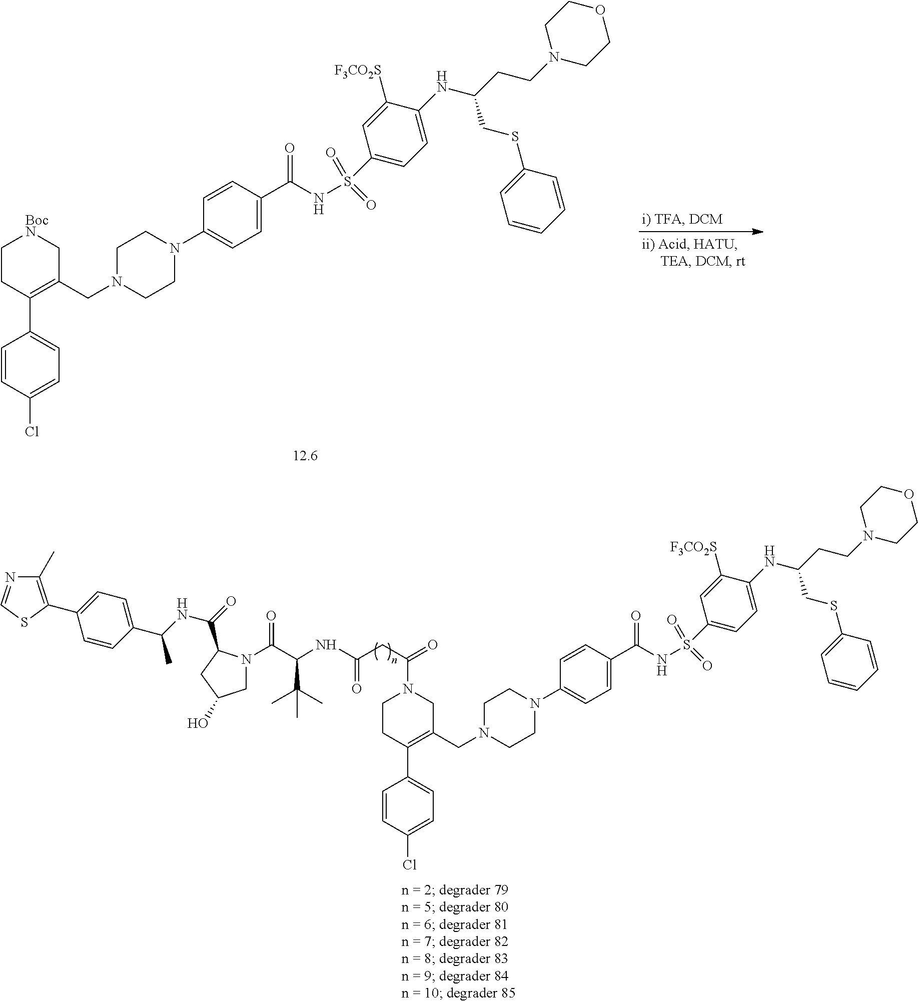

General procedure for the preparation of degraders #79-85: To a stirring solution of compound 12.6 was added TFA (10 equiv.) and the mixture was stirred for 3 h. The volatiles were removed under reduced pressure and the crude product was used in the next step without further purification. To a stirring solution of the crude amine salt (1.0 equiv.) and an acid (2.3-2.9) (1.1 equiv.) in DCM was added TEA (10 equiv.) at room temperature. To the mixture HATU (1.2 equiv.) was added and the reaction were stirred for 8 h at the same temperature. Upon completion of the reaction the solvent was removed under reduced pressure and the crude product was purified by flash column chromatography (DCM/MeOH/TEA=96:5:1). The product from column was mixed with 15 mL DCM and washed with saturated aqueous NH₄Cl solution. The organic portion was dried over Na₂SO₄, filtered, and concentrated under reduced pressure to afford the corresponding degrader.

(2S,4R)-1-((S)-2-(4-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-4-oxobutanamido)-3,3-dimethylbutanoyl)-4-hydroxy-N—((S)-1-(4-(4-methylthiazol-5-yl)phenyl)ethyl)pyrrolidine-2-carboxamide (degrader #79): ¹H NMR (600 MHz, CDCl₃) δ 8.71 (d, J=10.1 Hz, 1H), 8.33 (d, J=1.9 Hz, 1H), 8.15 (ddd, J=9.2, 4.2, 2.2 Hz, 1H), 7.95 (d, J=29.5 Hz, 1H), 7.67 (dd, J=18.5, 8.9 Hz, 2H), 7.43-7.37 (m, 6H), 7.32 (dd, J=7.9, 6.9 Hz, 2H), 7.28-7.21 (m, 3H), 7.06 (d, J=8.6 Hz, 1H), 6.89 (d, J=8.0 Hz, 1H), 6.86-6.77 (m, 3.5H), 6.69 (d, J=8.6 Hz, 0.5H), 6.63 (dd, J=9.5, 2.5 Hz, 1H), 5.12 (dd, J=13.4, 6.9 Hz, 1H), 4.76 (dt, J=28.4, 8.3 Hz, 1H), 4.62 (d, J=9.0 Hz, 0.5H), 4.46 (s, 0.5H), 4.42-4.36 (m, 1H), 4.25-4.19 (d, J=17.7 Hz, 0.5H), 4.10 (d, J=17.6 Hz, 0.5H), 4.06-3.99 (m, 1H), 3.92 (d, J=10.9 Hz, 2H), 3.88-3.76 (m, 1H), 3.68 (d, J=2.1 Hz, 4H), 3.59-3.53 (m, 1.5H), 3.33-3.18 (m, 4.5H), 3.12 (dd, J=14.0, 5.1 Hz, 1H), 3.04 (ddd, J=13.8, 7.1, 4.1 Hz, 1.5H), 2.95-2.79 (m, 3H), 2.79-2.57 (m, 3H), 2.51 (dd, J=8.9, 4.4 Hz, 3H), 2.48-2.27 (m, 12H), 2.18-2.10 (m, 2H), 2.03-1.95 (m, 1H), 1.70 (dd, J=13.6, 7.0 Hz, 1.5H), 1.52 (dd, J=6.8, 4.6 Hz, 3H), 1.07 (d, J=11.6 Hz, 9H). ESI⁺, m/z [M+H]⁺=1474.4.

(2S,4R)-1-((S)-2-(7-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-7-oxoheptanamido)-3,3-dimethylbutanoyl)-4-hydroxy-N—((S)-1-(4-(4-methylthiazol-5-yl)phenyl)ethyl)pyrrolidine-2-carboxamide (degrader #80): ¹H NMR (600 MHz, CDCl₃) δ 8.71 (t, J=7.6 Hz, 1H), 8.38-8.34 (m, 1H), 8.15 (d, J=9.3 Hz, 1H), 7.67 (d, J=8.3 Hz, 2H), 7.53-7.37 (m, 8H), 7.36-7.31 (m, 4H), 7.08 (d, J=8.6 Hz, 1H), 7.04-7.00 (m, 2H), 6.78 (dd, J=16.8, 9.1 Hz, 2H), 6.65 (t, J=9.7 Hz, 1H), 6.27 (dd, J=18.1, 9.8 Hz, 1H), 5.16-5.06 (m, 1H), 4.82-4.73 (m, 1H), 4.65-4.59 (m, 1H), 4.53 (d, J=21.7 Hz, 1H), 4.26 (s, 1H), 4.19-4.10 (m, 2H), 3.94 (s, 1H), 3.83-3.75 (m, 1H), 3.73-3.55 (m, 6H), 3.26 (s, 4H), 3.13 (dd, J=13.9, 5.0 Hz, 1H), 3.09-3.02 (m, 1H), 2.93 (d, J=11.0 Hz, 2H), 2.56-2.48 (m, 5H), 2.38 (ddd, J=24.8, 13.2, 7.2 Hz, 11H), 2.23-2.03 (m, 5H), 1.76-1.69 (m, 2H), 1.67-1.57 (m, 5H), 1.52-1.47 (m, 3H), 1.38 (ddd, J=21.8, 14.7, 7.4 Hz, 2H), 1.06 (s, 9H). ESI⁺, m/z [M+H]⁺=1515.4.

(2S,4R)-1-((S)-2-(8-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #81): ¹H NMR (600 MHz, CDCl₃) δ 8.71 (t, J=8.1 Hz, 1H), 8.36 (dd, J=4.6, 2.1 Hz, 1H), 8.15 (dd, J=9.2, 2.1 Hz, 1H), 7.71 (dd, J=53.3, 8.9 Hz, 2H), 7.55-7.36 (m, 7H), 7.32 (dd, J=12.0, 5.7 Hz, 4H), 7.12-6.94 (m, 3H), 6.79 (dd, J=34.6, 9.0 Hz, 2H), 6.64 (t, J=9.9 Hz, 1H), 6.28 (d, J=8.9 Hz, 1H), 5.17-5.05 (m, 1H), 4.81-4.46 (m, 3H), 4.32-4.10 (m, 3H), 3.97-3.87 (m, 1H), 3.86-3.72 (m, 2H), 3.71-3.53 (m, 6H), 3.24 (s, 4H), 3.13 (dd, J=13.9, 5.1 Hz, 1H), 3.05 (ddd, J=13.7, 7.1, 4.5 Hz, 1H), 2.96-2.89 (m, 2H), 2.55-2.50 (m, 3H), 2.46 (s, 2H), 2.45-2.30 (m, 12H), 2.16-2.08 (m, 2H), 2.07 (t, J=7.7 Hz, 1H), 1.72-1.62 (m, 4H), 1.57-1.46 (m, 4H), 1.40-1.29 (m, 5H), 1.07 (d, J=2.5 Hz, 9H). ESI⁺, m/z [M+H]⁺=1529.8.

(2S,4R)-1-((S)-2-(9-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide (degrader #82): ¹H NMR (600 MHz, CDCl₃) δ 8.71 (d, J=9.9 Hz, 1H), 8.41-8.26 (m, 1H), 8.14 (t, J=9.8 Hz, 1H), 7.71 (dd, J=84.6, 8.9 Hz, 2H), 7.61-7.37 (m, 7H), 7.36-7.30 (m, 4H), 7.09 (dd, J=15.0, 6.9 Hz, 1H), 7.03 (t, J=8.5 Hz, 2H), 6.79 (dd, J=31.8, 9.0 Hz, 2H), 6.64 (t, J=9.1 Hz, 1H), 6.26 (dd, J=42.7, 8.5 Hz, 1H), 5.18-5.07 (m, 1H), 4.83-4.44 (m, 3H), 4.33-4.10 (m, 3H), 3.87 (dd, J=29.7, 22.3 Hz, 2H), 3.78-3.47 (m, 7H), 3.24 (d, J=4.3 Hz, 4H), 3.13 (dd, J=13.9, 5.0 Hz, 1H), 3.05 (dd, J=13.8, 7.1 Hz, 1H), 2.93 (d, J=10.6 Hz, 2H), 2.54 (s, 3H), 2.46 (s, 2H), 2.39 (ddd, J=20.9, 11.9, 6.4 Hz, 14H), 2.17-2.10 (m, 2H), 2.06-1.98 (m, 1H), 1.72-1.63 (m, 4H), 1.51 (dd, J=12.9, 6.9 Hz, 3H), 1.48-1.43 (m, 1H), 1.39-1.31 (m, 5H), 1.07 (d, J=7.9 Hz, 9H). ESI⁺, m/z [M+H]⁺=1544.8.

(2S,4R)-1-((S)-2-(10-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-10-oxodecanamido)-3,3-dimethylbutanoyl)-4-hydroxy-N—((S)-1-(4-(4-methylthiazol-5-yl)phenyl)ethyl)pyrrolidine-2-carboxamide (degrader #83): ¹H NMR (600 MHz, CDCl₃) δ 8.70 (d, J=6.2 Hz, 1H), 8.39-8.25 (m, 1H), 8.13 (t, J=10.4 Hz, 1H), 7.74 (dd, J=94.6, 8.9 Hz, 2H), 7.53-7.37 (m, 6H), 7.33 (dt, J=9.9, 8.2 Hz, 5H), 7.08 (t, J=8.6 Hz, 1H), 7.03 (t, J=7.7 Hz, 2H), 6.78 (dd, J=19.5, 9.1 Hz, 2H), 6.64 (t, J=8.9 Hz, 1H), 6.36-6.16 (m, 1H), 5.12 (dd, J=14.9, 7.6 Hz, 1H), 4.86-4.47 (m, 3H), 4.32-4.19 (m, 2H), 4.14 (dt, J=24.4, 10.4 Hz, 1H), 3.97 (dt, J=22.9, 8.4 Hz, 2H), 3.74-3.58 (m, 7H), 3.29-3.19 (m, 4H), 3.12 (dd, J=13.8, 5.1 Hz, 1H), 3.05 (dd, J=13.8, 7.1 Hz, 1H), 2.94 (s, 2H), 2.54 (s, 3H), 2.48-2.29 (m, 14H), 2.12 (dd, J=19.6, 12.7 Hz, 2H), 1.75-1.58 (m, 6H), 1.50 (dd, J=14.3, 6.9 Hz, 3H), 1.43 (d, J=15.1 Hz, 2H), 1.38-1.29 (m, 5H), 1.20-1.15 (m, 2H), 1.07 (d, J=6.1 Hz, 9H). ESI⁺, m/z[M+H]⁺=1557.6.

(2S,4R)-1-((S)-2-(11-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-11-oxoundecanamido)-3,3-dimethylbutanoyl)-4-hydroxy-N—((S)-1-(4-(4-methylthiazol-5-yl)phenyl)ethyl)pyrrolidine-2-carboxamide (degrader #84): ¹H NMR (600 MHz, CDCl₃) δ 8.68 (s, 1H), 8.31 (dd, J=33.0, 1.8 Hz, 1H), 8.16-8.06 (m, 1H), 7.73 (dd, J=74.2, 8.9 Hz, 2H), 7.46-7.35 (m, 7H), 7.35-7.28 (m, 5H), 7.25 (t, J=4.7 Hz, 1H), 7.08-6.96 (m, 3H), 6.76 (dd, J=15.5, 9.0 Hz, 2H), 6.62 (t, J=9.7 Hz, 1H), 6.27 (dd, J=76.0, 8.7 Hz, 1H), 5.13-5.06 (m, 1H), 4.82-4.48 (m, 3H), 4.17 (ddd, J=33.1, 22.7, 13.3 Hz, 3H), 3.91 (s, 1H), 3.83-3.71 (m, 2H), 3.70-3.56 (m, 6H), 3.22 (s, 4H), 3.10 (dd, J=13.9, 5.1 Hz, 1H), 3.03 (dd, J=13.8, 7.1 Hz, 1H), 2.91 (s, 2H), 2.51 (d, J=6.8 Hz, 3H), 2.46-2.27 (m, 14H), 2.14-2.03 (m, 4H), 1.73-1.60 (m, 4H), 1.48 (dd, J=10.4, 7.1 Hz, 3H), 1.34 (dt, J=22.5, 7.4 Hz, 4H), 1.27 (s, 2H), 1.19 (d, J=7.0 Hz, 2H), 1.16-1.07 (m, 3H), 1.05 (d, J=9.7 Hz, 9H). ESI⁺, m/z[M+H]⁺=1571.

(2S,4R)-1-((S)-2-(12-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)-12-oxododecanamido)-3,3-dimethylbutanoyl)-4-hydroxy-N—((S)-1-(4-(4-methylthiazol-5-yl)phenyl)ethyl)pyrrolidine-2-carboxamide (degrader #85): ¹H NMR (600 MHz, CDCl₃) δ 8.69 (s, 1H), 8.33 (dd, J=31.9, 1.8 Hz, 1H), 8.17-8.08 (m, 1H), 7.76 (dd, J=63.9, 8.9 Hz, 2H), 7.46-7.36 (m, 7H), 7.35-7.29 (m, 5H), 7.10-6.94 (m, 3H), 6.77 (t, J=8.2 Hz, 2H), 6.64 (t, J=9.9 Hz, 1H), 6.45-6.19 (m, 1H), 5.14-5.05 (m, 1H), 4.82-4.68 (m, 2H), 4.54 (s, 1H), 4.23 (dd, J=34.1, 17.2 Hz, 2H), 4.14 (dd, J=11.9, 4.6 Hz, 1H), 3.98-3.86 (m, 2H), 3.66 (ddd, J=16.1, 14.1, 11.1 Hz, 7H), 3.25 (s, 4H), 3.12 (dd, J=13.8, 5.0 Hz, 1H), 3.04 (dd, J=13.8, 7.1 Hz, 1H), 2.93 (s, 2H), 2.54 (d, J=4.1 Hz, 3H), 2.38 (td, J=27.5, 14.1 Hz, 14H), 2.17-2.07 (m, 4H), 1.75-1.56 (m, 5H), 1.49 (d, J=6.9 Hz, 3H), 1.46 (d, J=6.2 Hz, 2H), 1.39-1.30 (m, 4H), 1.24-1.19 (m, 2H), 1.18-1.10 (m, 4H), 1.07 (d, J=9.1 Hz, 9H). ESI⁺, m/z [M+H]⁺=1585.

### Example 48: Preparation of Degraders #86 and #87

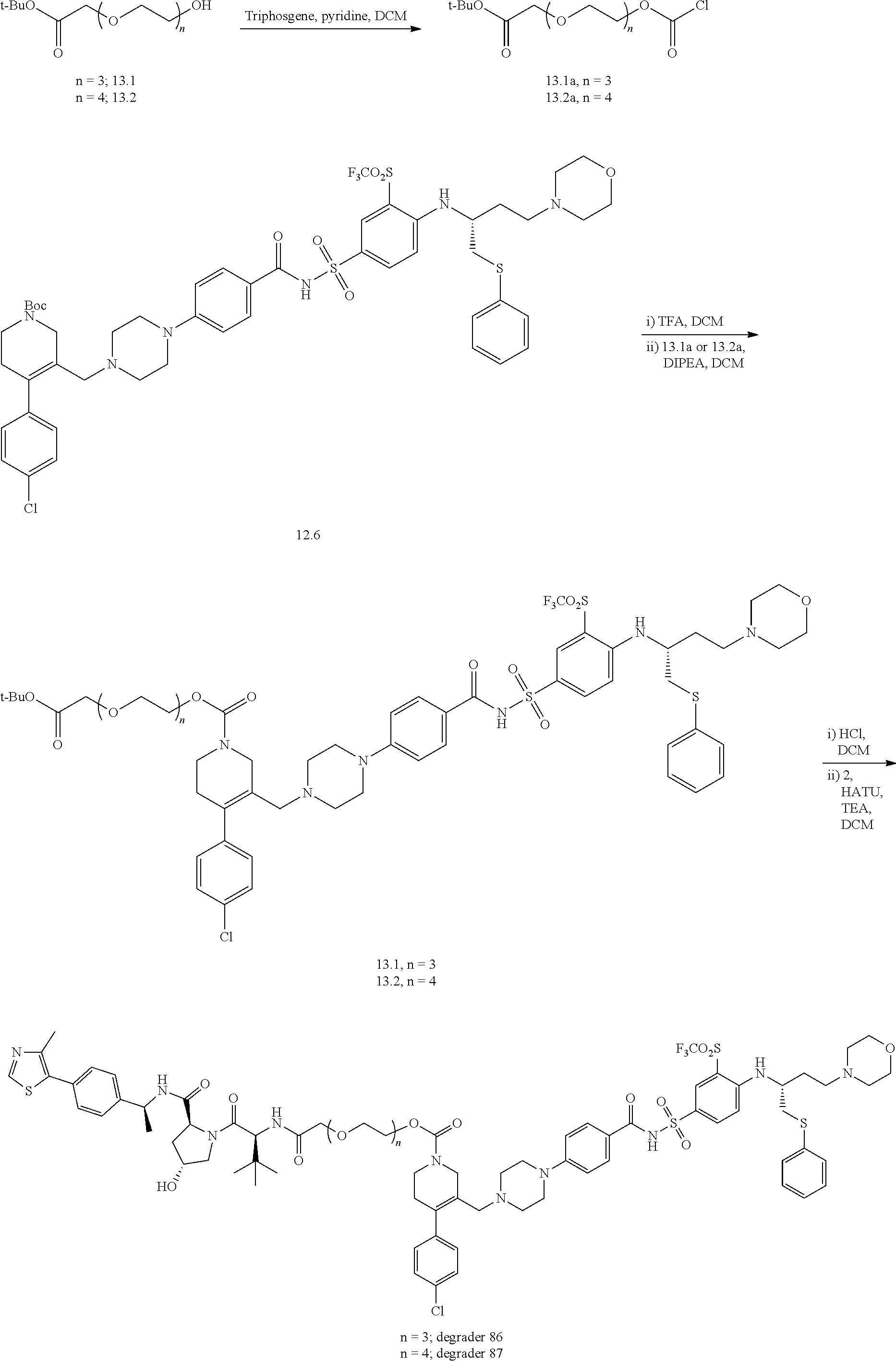

Preparation of compounds 13.3 and 13.4: To a solution of alcohols 13.1 or 13.2 (1 eq) in DCM was added triphosgene (0.5 equiv.) and pyridine (1.0 equiv.) at 0° C. The resulting mixture was warm to room temperature and stirred at room temperature for 2 h. The mixture was then diluted with ethyl acetate and washed with aqueous HCl solution, brine, and dried over sodium sulfate. Condensation of the mixture gave 13.1a and 13.2a, respectively, as a residue, which were used in the next step without further purification. TFA (20 equiv.) was added to a solution of compound 12.6 (1.0 equiv.) in DCM. After stirring at 0° C. for 1 h, the resulting mixture was condensed and added to a solution of compound 13.1a or 13.2a (2.0 equiv.) and DIPEA (6.0 equiv.) in DCM. The mixture was allowed to stir at room temperature for overnight and then condensed to give a residue which was chromatographed on silica gel to afford product 13.3 and 13.4, respectively.

13-dimethyl-11-oxo-3,6,9,12-tetraoxatetradecyl (R)-4-(4-chlorophenyl)-5-((4-(4-(((4-((4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridine-1(2H)-carboxylate (13.3): ¹H NMR (600 MHz, CDCl₃) δ 8.35 (s, 1H), 8.12 (d, J=8.8 Hz, 1H), 7.67 (d, J=24.4 Hz, 2H), 7.37 (d, J=7.4 Hz, 2H), 7.33-7.27 (m, 5H), 7.10-I 7.03 (m, 1H), 7.01 (d, J=8.4 Hz, 2H), 6.79 (s, 2H), 6.61 (d, J=9.3 Hz, 1H), 4.34-4.19 (m, 2H), 4.12 (s, 2H), 4.01 (s, 2H), 3.90 (s, 1H), 3.75-3.72 (m, 2H), 3.66 (dd, J=14.8, 9.6 Hz, 12H), 3.57 (s, 2H), 3.26 (s, 4H), 3.10 (dd, J=13.9, 5.0 Hz, 1H), 3.02 (dd, J=13.9, 7.2 Hz, 1H), 2.90 (s, 2H), 2.49-2.24 (m, 12H), 2.19-2.06 (m, 1H), 1.75-1.62 (m, 1H), 1.47 (s, 9H); ESI⁺, m/z [M+H]⁺=1237.3.

16-dimethyl-14-oxo-3,6,9,12,15-pentaoxaheptadecyl (R)-4-(4-chlorophenyl)-5-((4-(4-(((4-((4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridine-1(2H)-carboxylate (13.4): ¹H NMR (600 MHz, CDCl₃) δ 8.33 (d, J=1.6 Hz, 1H), 8.00 (dd, J=12.0, 5.1 Hz, 1H), 7.86 (s, 2H), 7.38 (dd, J=6.3, 5.0 Hz, 2H), 7.32-7.27 (m, 5H), 7.04-6.98 (m, 2H), 6.86 (s, 1H), 6.77 (d, J=8.0 Hz, 2H), 6.51 (dd, J=9.3, 5.7 Hz, 1H), 4.31-4.26 (m, 2H), 4.12 (s, 2H), 4.02 (s, 2H), 3.88-3.80 (m, 1H), 3.76-3.72 (m, 2H), 3.70-3.62 (m, 14H), 3.62-3.59 (m, 4H), 3.22 (s, 4H), 3.08 (dd, J=13.8, 4.8 Hz, 1H), 2.95 (dd, J=13.8, 7.8 Hz, 1H), 2.89 (s, 2H), 2.41 (s, 2H), 2.40-2.30 (m, 10H), 2.16-2.07 (m, 2H), 1.65-1.61 (m, 1H), 1.47 (s, 9H); ESI⁺, m/z [M+H]⁺=1281.3.

General procedure for the preparation of degraders #86 and #87: To a solution of t-butyl compound 13.3 or 13.4 (1.0 equiv.) in THF was added 4 N HCl solution in dioxane. The resulting mixture was stirred at room temperature for 2 h and then condensed under reduced pressure to give a residue which was then treated with TEA (3.0 equiv.), amine 2.0 (1.0 equiv.) and HATU (1.1 equiv.) in DCM for overnight. Solvent was removed under reduced pressure and the crude product was purified by flash column chromatography (DCM/MeOH/TEA=96:5:1). The product from column was mixed with 15 mL DCM and washed with saturated aqueous NH₄Cl solution. The organic portion was dried over Na₂SO₄, filtered, and concentrated under reduced pressure to afford the corresponding degrader.

(S)-13-((2S,4R)-4-hydroxy-2-(((S)-1-(4-(4-methylthiazol-5-yl)phenyl)ethyl)carbamoyl)pyrrolidine-1-carbonyl)-14,14-dimethyl-11-oxo-3,6,9-trioxa-12-azapentadecyl 4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridine-1(2H)-carboxylate (degrader #86): ¹H NMR (600 MHz, CDCl₃) δ 8.71 (s, 1H), 8.36 (s, 1H), 8.14 (d, J=9.0 Hz, 1H), 7.77-7.70 (m, 2H), 7.58-7.52 (m, 1H), 7.40 (ddd, J=9.1, 8.2, 6.4 Hz, 6H), 7.33 (dd, J=9.8, 4.6 Hz, 5H), 7.29 (d, J=1.2 Hz, 1H), 7.08 (d, J=8.5 Hz, 1H), 7.05-7.01 (m, 2H), 6.80 (d, J=7.3 Hz, 2H), 6.65 (d, J=8.9 Hz, 1H), 5.13 (dd, J=13.2, 6.0 Hz, 1H), 4.79 (s, 1H), 4.67 (s, 1H), 4.56 (s, 1H), 4.33 (dt, J=8.9, 5.8 Hz, 3H), 4.27-4.17 (m, 1H), 4.15 (s, 2H), 4.01 (p, J=5.0 Hz, 2H), 3.93 (s, 1H), 3.74 (d, J=4.2 Hz, 2H), 3.72-3.64 (m, 10H), 3.62-3.53 (m, 2H), 3.31-3.22 (m, 4H), 3.13 (dd, J=13.9, 5.1 Hz, 1H), 3.05 (dd, J=13.9, 7.1 Hz, 1H), 2.93 (s, 2H), 2.52 (s, 3H), 2.45 (s, 2H), 2.43-2.33 (m, 10H), 2.18-2.09 (m, 2H), 1.51 (d, J=6.9 Hz, 3H), 1.39-1.35 (m, 4H), 1.09 (s, 9H). ESI⁺, m/z [M+H]⁺=1607.5.

(S)-16-((2S,4R)-4-hydroxy-2-(((S)-1-(4-(4-methylthiazol-5-yl)phenyl)ethyl)carbamoyl)pyrrolidine-1-carbonyl)-17,17-dimethyl-14-oxo-3,6,9,12-tetraoxa-15-azaoctadecyl 4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridine-1(2H)-carboxylate (degrader #87): ¹H NMR (600 MHz, CDCl₃) δ 8.70 (s, 1H), 8.35 (s, 1H), 8.13 (dd, J=9.2, 2.0 Hz, 1H), 7.76 (s, 1H), 7.73-7.64 (m, 1H), 7.40 (dt, J=17.9, 8.4 Hz, 6H), 7.36-7.31 (m, 6H), 7.06 (d, J=8.6 Hz, 1H), 7.03 (d, J=8.4 Hz, 2H), 6.79 (d, J=8.5 Hz, 2H), 6.63 (d, J=9.3 Hz, 1H), 5.16-5.06 (m, 1H), 4.77 (t, J=7.9 Hz, 1H), 4.66 (s, 1H), 4.54 (s, 1H), 4.36-4.28 (m, 2H), 4.16 (d, J=18.6 Hz, 3H), 3.97 (s, 1H), 3.92 (dd, J=7.7, 4.2 Hz, 2H), 3.75 (dd, J=8.3, 3.5 Hz, 2H), 3.68 (dd, J=10.6, 5.1 Hz, 12H), 3.59 (s, 4H), 3.27 (d, J=4.7 Hz, 4H), 3.12 (dd, J=13.9, 5.0 Hz, 1H), 3.04 (dd, J=13.9, 7.3 Hz, 1H), 2.92 (s, 2H), 2.53 (s, 3H), 2.44 (s, 2H), 2.37 (dd, J=12.0, 6.3 Hz, 8H), 2.34-2.29 (m, 2H), 2.12 (dd, J=18.1, 12.5 Hz, 2H), 1.74-1.64 (m, 4H), 1.49 (d, J=6.9 Hz, 3H), 1.35 (t, J=7.3 Hz, 2H), 1.09 (s, 9H). ESI⁺, m/z [M+H]⁺=1651.

### Example 49: Preparation of Degrader #88

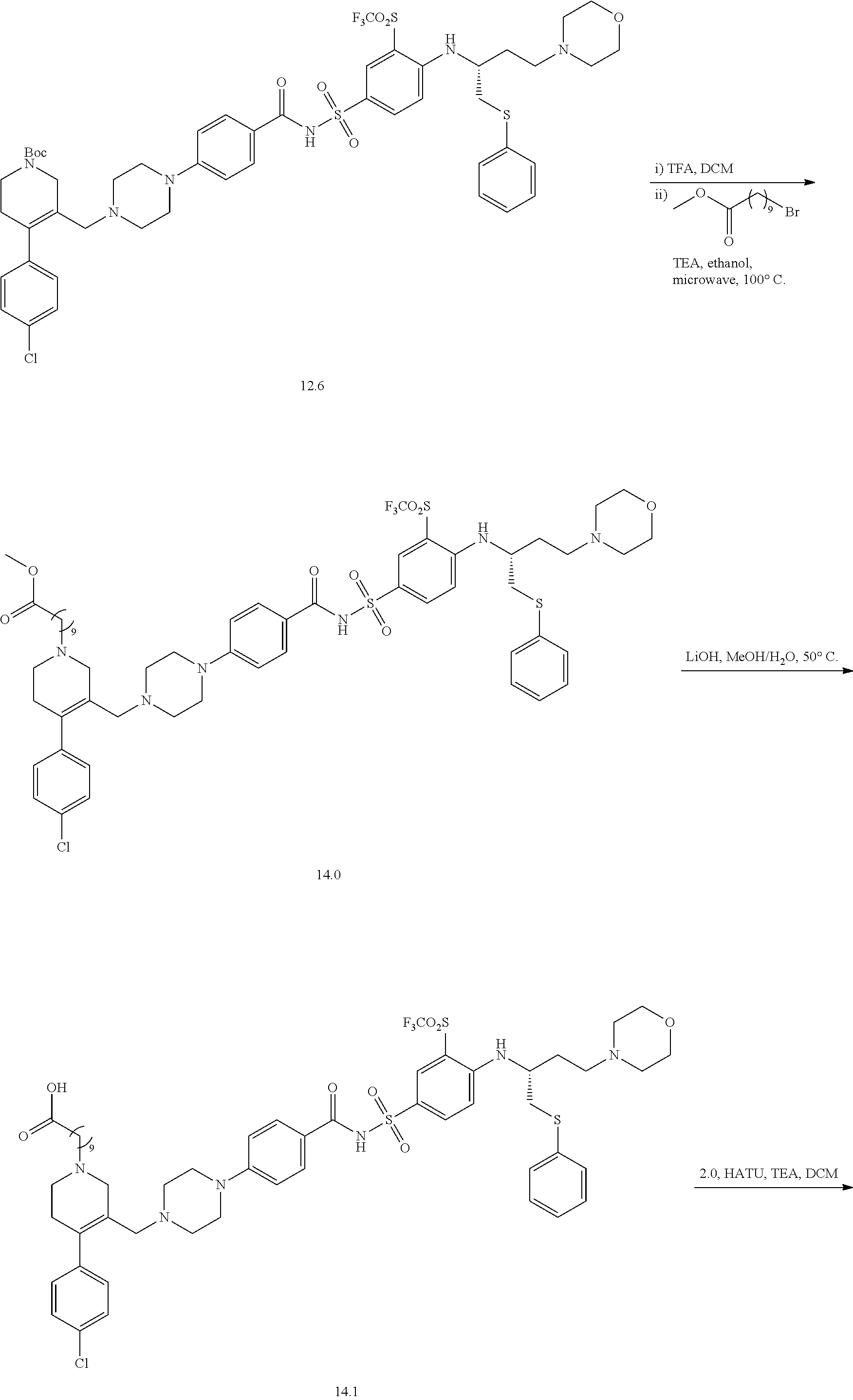

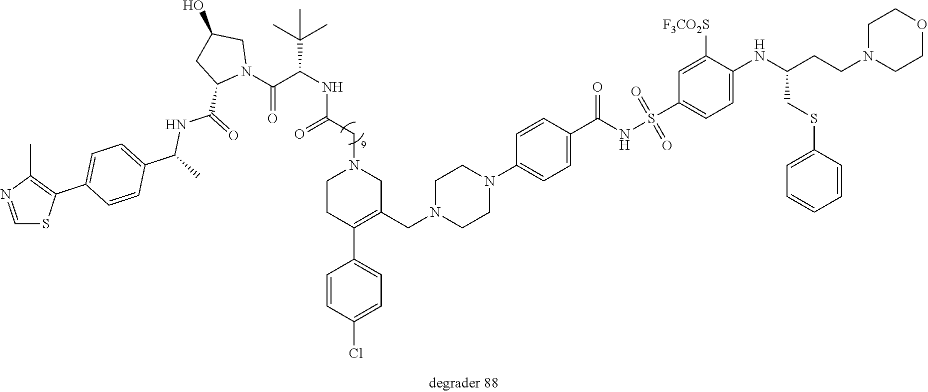

Preparation of methyl (R)-10-(4-(4-chlorophenyl)-5-((4-(4-(((4-((4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)decanoate (14.0): TFA (0.5 mL) was added to a solution of compound 12.6 (50 mg, 0.053 mmol) in DCM (1 mL). After stirring at 0° C. for 1 h, the resulting mixture was condensed to afford a residue which was mixed with TEA (44 μL, 0.318 mmol) and bromo ester (44 mg, 0.159 mmol) in ethanol (3 mL). The resulting mixture was stirred under microwave radiation at 100° C. for 2-4 h. Removal of the solvent under reduced pressure afforded a residue which was chromatographed on silica gel to yield the title compound (36 mg, 61% yield). ¹H NMR (600 MHz, CDCl₃) δ 8.36 (s, 1H), 8.06 (d, J=8.7 Hz, 1H), 7.87 (d, J=8.6 Hz, 2H), 7.37 (d, J=7.4 Hz, 2H), 7.34-7.27 (m, 4H), 7.24 (t, J=7.2 Hz, 1H), 7.07 (d, J=8.2 Hz, 2H), 6.92 (d, J=7.5 Hz, 1H), 6.75 (d, J=8.5 Hz, 2H), 6.56 (d, J=9.3 Hz, 1H), 3.87 (s, 1H), 3.73-3.65 (m, 5H), 3.65 (s, 3H), 3.31-3.20 (m, 5H), 3.18-3.06 (m, 4H), 3.04-2.95 (m, 3H), 2.67-2.33 (m, 10H), 2.27 (t, J=7.5 Hz, 2H), 2.19-2.11 (m, 1H), 1.98-1.88 (m, 2H), 1.75-1.65 (m, 1H), 1.64-1.53 (m, 2H), 1.38-1.18 (m, 14H). ESI⁺, m/z [M+H]⁺=1131.5.

Preparation of (R)-10-(4-(4-chlorophenyl)-5-((4-(4-(((4-((4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)decanoic acid (14.1): To a solution of ester 14.0 (36 mg, 0.032 mmol) in methanol was added 3N LiOH (0.2 mL, 0.64 mmol) aqueous solution. After stirring at 50° C. for 2 h, the mixture was cooled down in ice bath and the pH was adjusted to <3.0 with 1N HCl. The mixture was extracted with DCM for 3 times and the combined organic layers were washed with water and brine, dried over anhydrous sodium sulfate, filtered, and condensed to afford residue which was purified through a column packed with silica gel to yield product 14.1 (25 mg, 70% yield). ¹H NMR (600 MHz, CD₃OD) δ 8.25 (s, 1H), 8.02-7.97 (m, 1H), 7.92 (d, J=8.6 Hz, 2H), 7.41 (d, J=8.4 Hz, 2H), 7.38-7.34 (m, 2H), 7.26-7.20 (m, 4H), 7.17 (t, J=7.4 Hz, 1H), 6.87 (dd, J=18.3, 9.2 Hz, 3H), 4.05 (dd, J=8.3, 4.8 Hz, 1H), 3.97 (s, 2H), 3.65 (dd, J=12.1, 6.0 Hz, 4H), 3.51 (s, 2H), 3.28-3.25 (m, 4H), 3.18 (dd, J=14.2, 5.9 Hz, 1H), 3.00 (s, 2H), 2.75 (s, 2H), 2.59-2.40 (m, 9H), 2.23 (t, J=7.5 Hz, 2H), 2.11 (dt, J=12.4, 7.8 Hz, 1H), 1.89-1.83 (m, 2H), 1.83-1.77 (m, 1H), 1.60-1.55 (m, 2H), 1.42 (dd, J=25.7, 9.4 Hz, 4H), 1.34 (t, J=7.3 Hz, 10H). ESI⁺, m/z [M+H]⁺=1117.7.

Preparation of (2S,4R)-1-((S)-2-(10-(4-(4-chlorophenyl)-5-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-3,6-dihydropyridin-1(2H)-yl)decanamido)-3,3-dimethylbutanoyl)-4-hydroxy-N—((R)-1-(4-(4-methylthiazol-5-yl)phenyl)ethyl)pyrrolidine-2-carboxamide (degrader 88): To a solution of 14.1 (5 mg, 4.5 μmol) in DCM (1.5 mL) was added amine 2.0 (2.1 mg, 4.5 μmol), TEA (1.37 μL, 9.9 μmol) and HATU (1.87 mg, 4.95 mmol). After stirring at room temperature overnight, the mixture was condensed and purified through preparation TLC to afford degrader #88 (5.5 mg, 80% yield). ¹H NMR (600 MHz, CDCl₃) δ 8.70 (s, 1H), 8.35 (s, 1H), 8.12-8.04 (s, 1H), 7.90-7.80 (s, 2H), 7.43-7.37 (m, 7H), 7.35-7.30 (m, 4H), 7.28-7.25 (m, 1H), 7.09 (d, J=8.0 Hz, 2H), 6.96 (s, 1H), 6.80 (d, J=8.5 Hz, 2H), 6.59 (d, J=9.1 Hz, 1H), 6.32 (s, 1H), 5.16-5.07 (m, 1H), 4.73 (t, J=7.9 Hz, 1H), 4.66 (s, 1H), 4.53 (s, 1H), 4.14-4.08 (m, 1H), 3.94-3.86 (s, 1H), 3.73-3.64 (m, 5H), 3.63-3.58 (m, 1H), 3.23 (s, 5H), 3.16-3.07 (m, 4H), 3.01 (dd, J=13.8, 7.5 Hz, 1H), 2.97 (s, 2H), 2.53 (s, 3H), 2.52-2.30 (m, 10H), 2.24-2.19 (m, 1H), 2.18-2.10 (m, 3H), 1.88 (s, 2H), 1.70-1.66 (m, 1H), 1.53 (s, 2H), 1.50 (d, J=6.9 Hz, 3H), 1.41-1.26 (m, 14H), 1.07 (s, 9H); ESI⁺, m/z [M+H]⁺=1543.1.

### Example 50: Preparation of Degrader #89-91

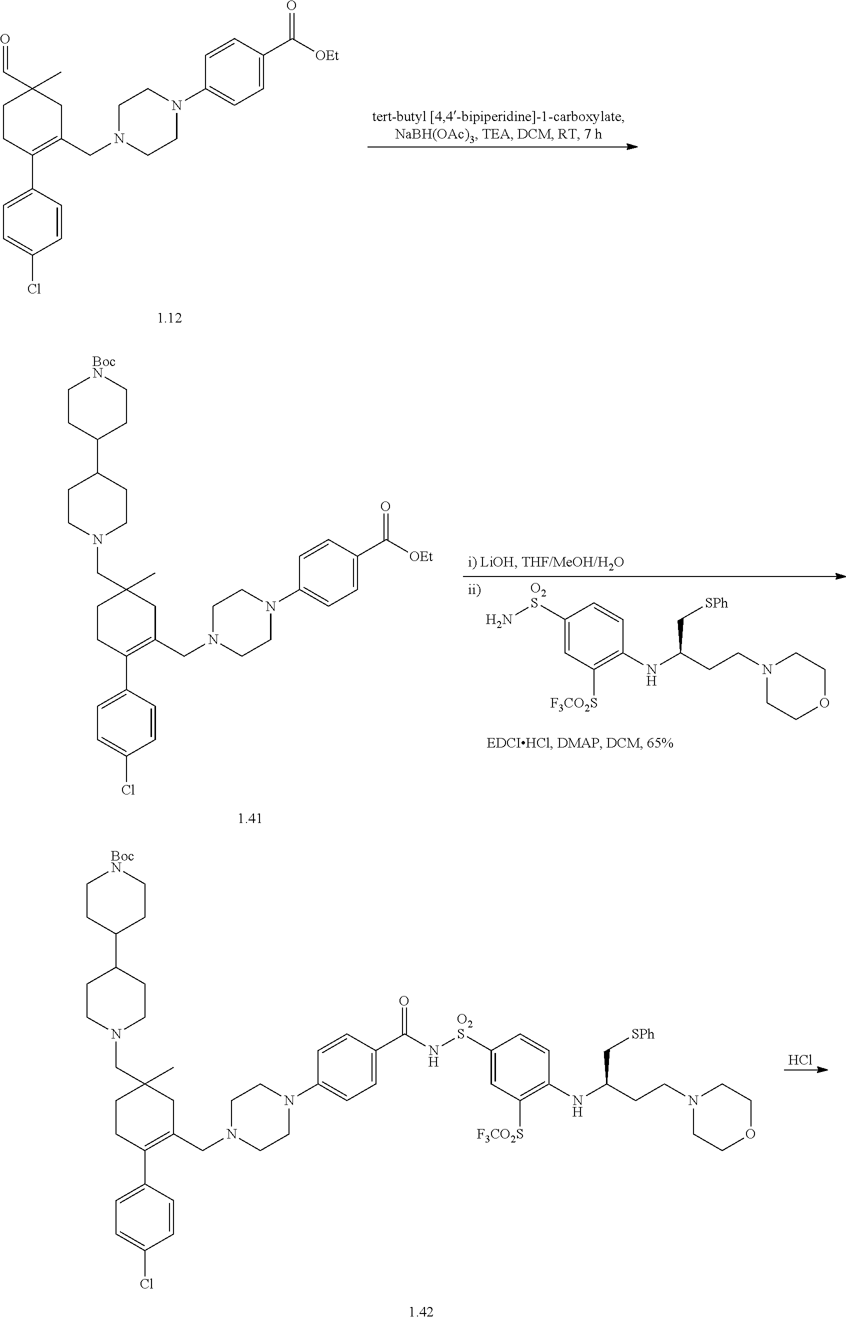

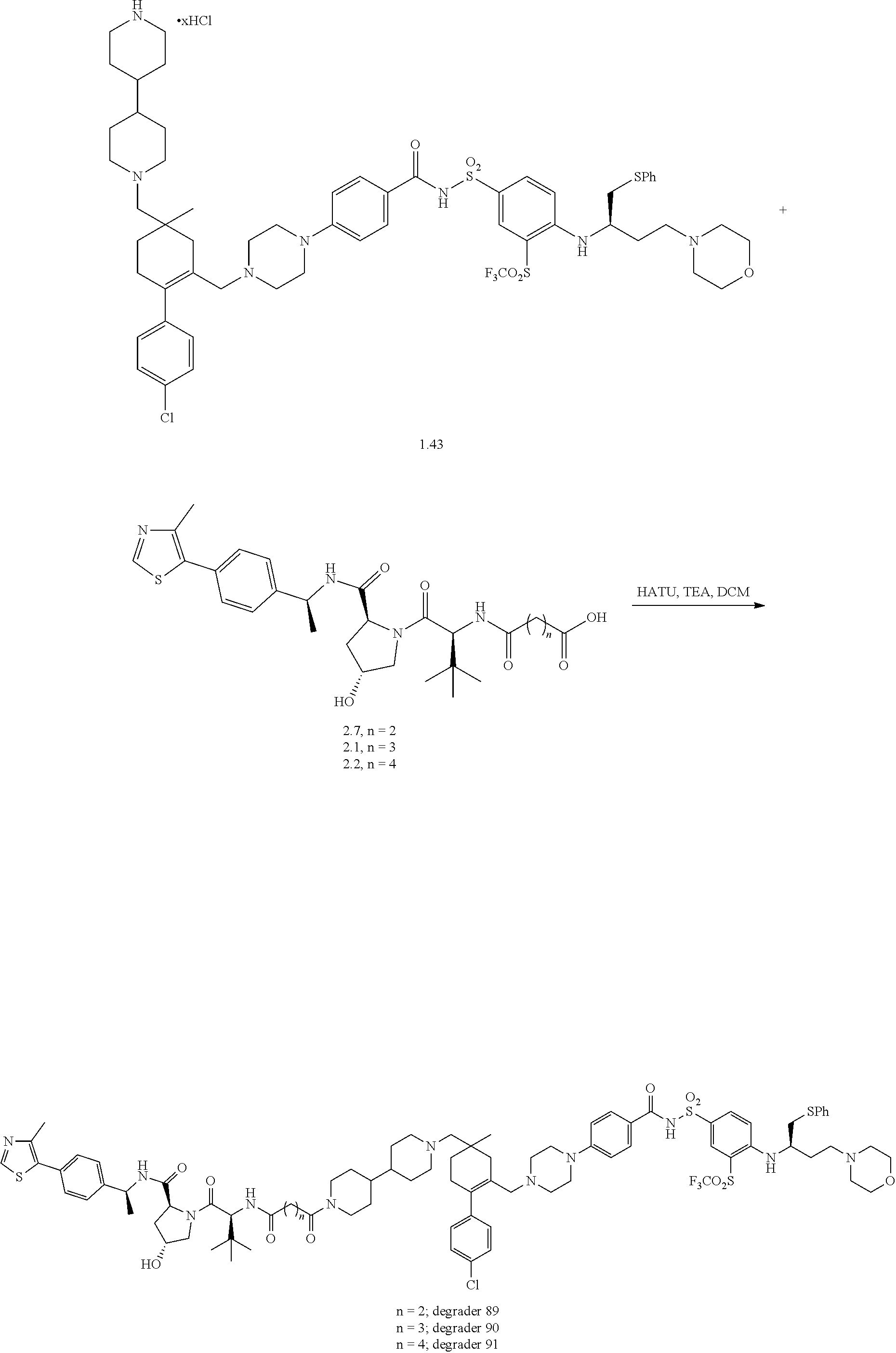

Degraders #89-91 were prepared from aldehyde 1.12 by following the same synthetic protocol as degrader #46 was prepared from aldehyde 1.12, with tert-butyl [4,4-bipiperidine]-1-carboxylate was used in place of tert-butyl (S)-piperidin-3-ylcarbamate in the synthetic sequence.

tert-butyl 1′-((4′-chloro-6-((4-(4-(ethoxycarbonyl)phenyl)piperazin-1-yl)methyl)-4-methyl-2,3,4,5-tetrahydro-[1,1′-biphenyl]-4-yl)methyl)-[4,4′-bipiperidine]-1-carboxylate (1.41): ¹H NMR (600 MHz, Chloroform-d) δ 7.89 (d, J=9.0 Hz, 2H), 7.27 (d, J=8.4 Hz, 2H), 6.99 (d, J=8.4 Hz, 2H), 6.81 (d, J=9.0 Hz, 2H), 4.32 (q, J=7.1 Hz, 2H), 3.25 (t, J=5.2 Hz, 4H), 2.83 (d, J=10.8 Hz, 2H), 2.79 (s, 2H), 2.62 (s, 2H), 2.35 (qt, J=10.9, 4.9 Hz, 4H), 2.30-2.24 (m, 1H), 2.21 (d, J=8.5 Hz, 1H), 2.18-2.14 (m, 3H), 2.11 (s, 1H), 1.89 (d, J=17.3 Hz, 1H), 1.66 (d, J=12.1 Hz, 2H), 1.63-1.60 (m, 4H), 1.58-1.52 (m, 3H), 1.45 (s, 9H), 1.36 (t, J=7.1 Hz, 3H), 1.28-1.24 (m, 2H), 1.20-1.16 (m, 1H), 1.14-1.08 (m, 2H), 1.02-0.98 (m, 1H), 0.93 (s, 3H).

tert-butyl 1′-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-[4,4′-bipiperidine]-1-carboxylate (1.42): ¹H NMR (600 MHz, Chloroform-d) δ 8.34 (d, J=2.2 Hz, 1H), 8.01 (d, J=8.7 Hz, 1H), 7.84 (d, J=8.4 Hz, 2H), 7.37 (d, J=7.5 Hz, 2H), 7.31-7.27 (m, 4H), 7.01 (d, J=8.0 Hz, 2H), 6.85 (d, J=8.5 Hz, 1H), 6.71-6.63 (m, 2H), 6.53 (d, J=9.0 Hz, 1H), 4.07 (dd, J=14.7, 7.8 Hz, 2H), 3.84 (dd, J=10.5, 4.3 Hz, 1H), 3.64 (t, J=8.1 Hz, 5H), 3.33-3.16 (m, 6H), 2.98 (d, J=12.2 Hz, 2H), 2.73-2.46 (m, 9H), 2.46-2.24 (m, 13H), 2.11 (d, J=14.1 Hz, 2H), 1.69 (d, J=15.2 Hz, 3H), 1.62 (dd, J=13.2, 5.3 Hz, 6H), 1.43 (s, 9H), 1.10 (s, 3H), 1.06 (d, J=5.3 Hz, 3H).

(2S,4R)-1-((2S)-2-(4-(1′-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-[4,4′-bipiperidin]-1-yl)-4-oxobutanamido)-3,3-dimethylbutanoyl)-4-hydroxy-N—((S)-1-(4-(4-methylthiazol-5-yl)phenyl)ethyl)pyrrolidine-2-carboxamide (degrader #89): ¹H NMR (600 MHz, Chloroform-d) δ 8.66 (d, J=9.4 Hz, 1H), 8.33-8.28 (m, 1H), 8.09 (s, 1H), 7.83-7.74 (m, 2H), 7.64-7.51 (m, 1H), 7.41-7.32 (m, 6H), 7.32-7.27 (m, 4H), 6.99 (d, J=8.0 Hz, 4H), 6.73 (s, 2H), 6.58 (t, J=12.5 Hz, 1H), 5.11-5.00 (m, 1H), 4.76 (d, J=8.9 Hz, 1H), 4.52-4.42 (m, 2H), 4.10 (dd, J=22.1, 15.1 Hz, 1H), 3.84 (d, J=38.8 Hz, 2H), 3.64 (s, 4H), 3.53 (d, J=12.6 Hz, 1H), 3.20 (s, 3H), 3.12-3.07 (m, 1H), 3.02-2.98 (m, 1H), 2.92-2.82 (m, 2H), 2.60-2.48 (m, 7H), 2.44-2.40 (m, 3H), 2.31 (ddt, J=28.7, 15.1, 7.6 Hz, 9H), 2.16-2.05 (m, 3H), 1.76-1.57 (m, 14H), 1.25 (s, 15H), 1.06 (s, 12H).

(2S,4R)-1-((2S)-2-(5-(1′-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-[4,4′-bipiperidin]-1-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide (degrader #90): ¹H NMR (600 MHz, Chloroform-d) δ 8.66 (s, 1H), 8.34 (d, J=7.0 Hz, 1H), 8.06 (t, J=8.3 Hz, 1H), 7.80 (d, J=7.6 Hz, 2H), 7.36 (dd, J=14.2, 8.4 Hz, 6H), 7.30 (t, J=7.1 Hz, 4H), 7.26-7.23 (m, 3H), 7.03 (s, 2H), 6.93-6.88 (m, 1H), 6.64 (s, 1H), 6.59 (d, J=9.2 Hz, 1H), 5.07 (dt, J=13.7, 6.9 Hz, 1H), 4.77-4.67 (m, 1H), 4.50 (dd, J=26.0, 7.5 Hz, 3H), 4.14-4.07 (m, 1H), 3.88 (s, 1H), 3.78 (s, 1H), 3.65 (q, J=5.9 Hz, 4H), 3.57 (dd, J=11.6, 3.9 Hz, 1H), 3.22 (s, 4H), 3.11 (dd, J=11.9, 7.8 Hz, 2H), 3.00 (d, J=6.5 Hz, 1H), 2.86 (s, 3H), 2.64 (s, 3H), 2.51 (s, 3H), 2.46-2.26 (m, 19H), 2.15-2.07 (m, 3H), 1.91-1.81 (m, 3H), 1.67-1.54 (m, 5H), 1.45 (ddd, J=16.5, 8.3, 4.9 Hz, 5H), 1.28-1.23 (m, 4H), 1.21-1.12 (m, 2H), 1.07-1.03 (m, 14H).

(2S,4R)-1-((2S)-2-(6-(1′-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-[4,4′-bipiperidin]-1-yl)-6-oxohexanamido)-3,3-dimethylbutanoyl)-4-hydroxy-N—((S)-1-(4-(4-methylthiazol-5-yl)phenyl)ethyl)pyrrolidine-2-carboxamide (degrader #91): ¹H NMR (600 MHz, Chloroform-d) δ 8.66 (s, 1H), 8.35-8.31 (m, 1H), 8.06 (d, J=9.0 Hz, 1H), 7.79 (d, J=8.0 Hz, 2H), 7.51-7.43 (m, 1H), 7.41-7.34 (m, 6H), 7.29 (q, J=6.9, 6.2 Hz, 5H), 7.01 (d, J=7.9 Hz, 2H), 6.92 (s, 1H), 6.66 (s, 2H), 6.58 (d, J=8.6 Hz, 1H), 6.49 (d, J=9.7 Hz, 1H), 5.07 (q, J=6.5, 5.8 Hz, 1H), 4.75 (td, J=8.3, 3.8 Hz, 1H), 4.61-4.56 (m, 1H), 4.52 (d, J=14.2 Hz, 1H), 4.48 (s, 1H), 4.14-4.04 (m, 1H), 3.88 (d, J=12.4 Hz, 1H), 3.77 (s, 1H), 3.68-3.61 (m, 4H), 3.58 (dd, J=11.1, 3.8 Hz, 1H), 3.21 (s, 4H), 3.14-3.08 (m, 2H), 3.01-2.97 (m, 1H), 2.88 (d, J=13.7 Hz, 2H), 2.52-2.51 (m, 3H), 2.49 (s, 2H), 2.42 (s, 4H), 2.40-2.33 (m, 6H), 2.31 (s, 5H), 2.26 (s, 4H), 2.23-2.15 (m, 3H), 2.10 (s, 4H), 1.69-1.57 (m, 9H), 1.46 (dd, J=6.9, 2.4 Hz, 3H), 1.42 (d, J=6.9 Hz, 1H), 1.25 (d, J=2.5 Hz, 3H), 1.22-1.08 (m, 3H), 1.05 (d, J=3.8 Hz, 13H).

### Example 51: Preparation of Degrader #92

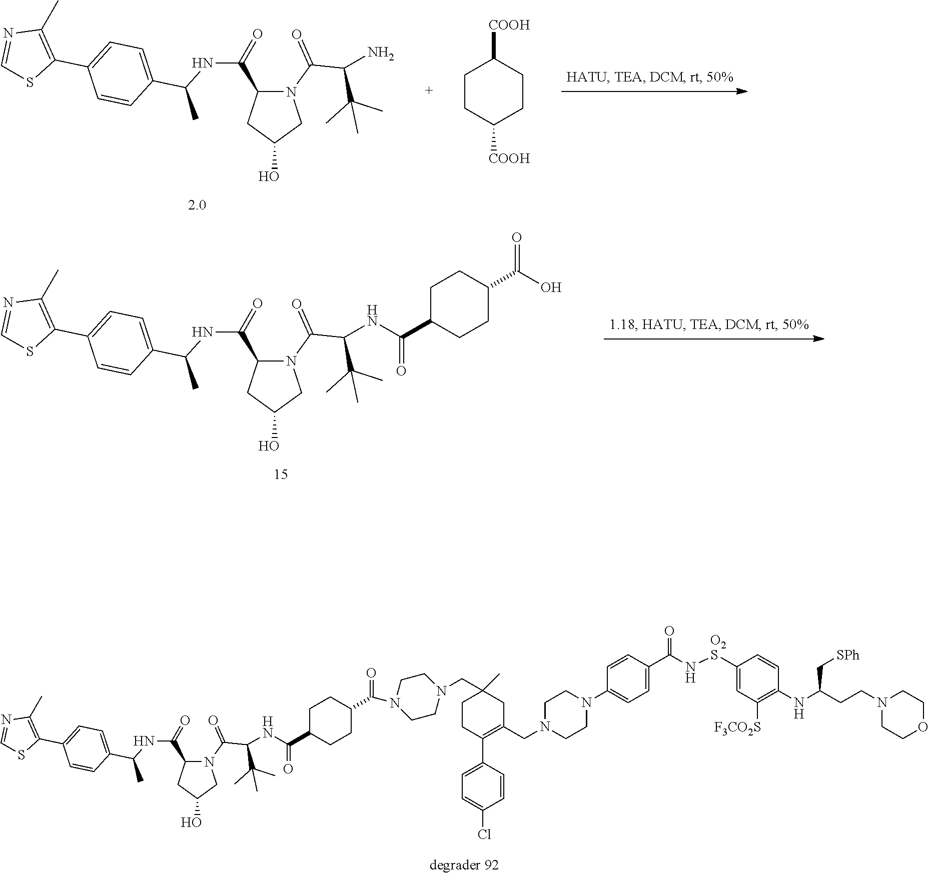

(1,4-trans)-4-(((S)-1-((2S,4R)-4-Hydroxy-2-(((S)-1-(4-(4-methylthiazol-5-yl)phenyl)ethyl)carbamoyl)pyrrolidin-1-yl)-3,3-dimethyl-1-oxobutan-2-yl)carbamoyl)cyclohexane-1-carboxylic acid (15): A mixture of amine 2.0 (1.0 equiv.), trans-1,4-Cyclohexanedicarboxylic acid (1.1 equiv.), HATU (1.2 equiv.) and TEA (5.0 equiv.) was taken in DCM and the reaction mixture was stirred at room temperature for 4 h. After completion of the reaction, DCM was evaporated and the crude product was purified by column chromatography. ¹H NMR (600 MHz, Chloroform-d) δ 8.67 (s, 1H), 7.45 (d, J=7.8 Hz, 1H), 7.40 (d, J=8.2 Hz, 2H), 7.36 (d, J=8.3 Hz, 2H), 6.21 (d, J=8.6 Hz, 1H), 5.07 (p, J=7.0 Hz, 1H), 4.75 (t, J=7.9 Hz, 1H), 4.54 (d, J=8.7 Hz, 1H), 4.51 (s, 1H), 4.14 (d, J=11.4 Hz, 1H), 3.57 (dd, J=11.4, 3.6 Hz, 1H), 2.56 (ddd, J=12.6, 7.3, 4.8 Hz, 1H), 2.53 (s, 3H), 2.19-2.10 (m, 2H), 2.10-2.03 (m, 3H), 1.92 (t, J=13.5 Hz, 2H), 1.47 (d, J=7.0 Hz, 3H), 1.45-1.38 (m, 3H), 1.04 (s, 9H).

(2S,4R)-1-((2S)-2-((1,4-trans)-4-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazine-1-carbonyl)cyclohexane-1-carboxamido)-3,3-dimethylbutanoyl)-4-hydroxy-N—((S)-1-(4-(4-methylthiazol-5-yl)phenyl)ethyl)pyrrolidine-2-carboxamide (degrader #92): To a stirring solution of amines 1.18 (12 mg, 0.011 mmol) and acid 15 (7 mg, 0.012 mmol) in DCM (1 mL) was added TEA (0.01 mL, 0.066 mmol) at room temperature. To the mixture HATU (5 mg, 0.012 mmol) was added and the reaction were stirred for 8 h at the same temperature. Upon completion of the reaction, solvent was removed under reduced pressure and the crude product was purified by flash column chromatography (DCM/MeOH=96:4). The product from column was mixed with 15 mL DCM and washed with saturated aqueous NH₄Cl. The organic portion was dried over Na₂SO₄, filtered, and DCM was evaporated under reduced pressure to afford the title compound. ¹H NMR (600 MHz, Chloroform-d) δ 8.68 (s, 1H), 8.29 (t, J=1.8 Hz, 1H), 8.09 (ddd, J=15.0, 9.2, 2.3 Hz, 1H), 7.78 (dd, J=21.5, 8.6 Hz, 2H), 7.43-7.33 (m, 7H), 7.32-7.27 (m, 4H), 7.04 (d, J=8.5 Hz, 1H), 6.98 (dd, J=8.4, 2.5 Hz, 2H), 6.77 (dd, J=9.0, 5.0 Hz, 2H), 6.61 (d, J=9.4 Hz, 1H), 6.42 (dd, J=34.8, 8.9 Hz, 1H), 5.09 (p, J=7.1 Hz, 1H), 4.81 (dd, J=20.2, 8.7 Hz, 1H), 4.75 (q, J=7.9 Hz, 1H), 4.51 (d, J=15.2 Hz, 1H), 4.25-4.16 (m, 1H), 3.93 (dd, J=32.8, 10.6 Hz, 2H), 3.65 (s, 4H), 3.60-3.54 (m, 1H), 3.43 (d, J=9.2 Hz, 1H), 3.37-3.22 (m, 2H), 3.17 (s, 4H), 3.12-3.08 (m, 1H), 3.02 (dd, J=13.5, 6.8 Hz, 1H), 2.92-2.71 (m, 2H), 2.57 (dd, J=9.4, 4.3 Hz, 1H), 2.52 (d, J=1.2 Hz, 3H), 2.45-2.24 (m, 14H), 2.24-1.98 (m, 5H), 1.97-1.62 (m, 6H), 1.55 (dt, J=22.0, 11.5 Hz, 3H), 1.47 (dd, J=7.0, 3.1 Hz, 5H), 1.45-1.28 (m, 3H), 1.25 (s, 2H), 1.06 (s, 9H), 0.98 (d, J=5.0 Hz, 3H).

### Example 52: Preparation of Degrader #93

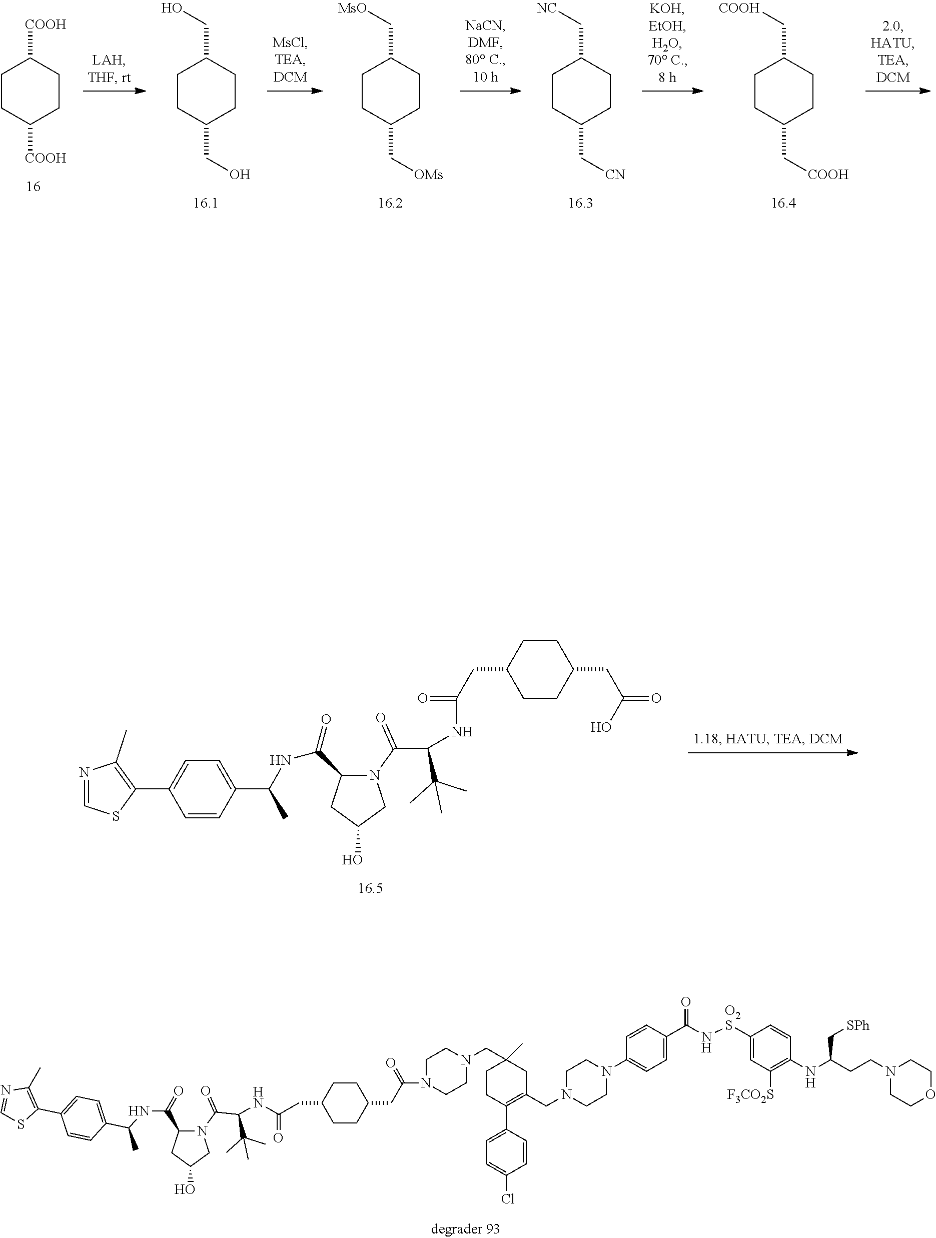

Degrader 93 was synthesized from diol 16.1 following the same synthetic sequence as degrader 40 was synthesized from diol 7.0. Diol 16.1 was synthesized from commercially available compound 16.

(1,4-cis-Cyclohexanediyl)dimethanol (16.1): Compound 16 (1 equiv.) was dissolved in THF followed by the addition of LAH (2 equiv.). The reaction was stirred at room temperature overnight. Upon completion of the reaction 2N NaOH (2 mL) solution was added to the reaction mixture followed by addition of 2 mL water. Upon stirring the mixture for 4 h, solid MgSO₄ was added and the reaction mixture was stirred overnight. The solution was filtered through celite and washed with EtOAc several times. The filtrate was concentrated and purified by flash column chromatography (Hexane/EtOAc=50:50) to get the desired compound 16.1. ¹H NMR (600 MHz, Chloroform-d) δ 3.54 (d, J=7.0 Hz, 4H), 1.69 (ddt, J=11.5, 6.0, 3.0 Hz, 2H), 1.54 (ddt, J=8.5, 7.1, 2.8 Hz, 4H), 1.42 (tt, J=10.6, 6.1 Hz, 4H).

(1,4-cis-cyclohexanediyl)bis(methylene) dimethanesulfonate (16.2): ¹H NMR (600 MHz, Chloroform-d) δ 4.13 (d, J=7.1 Hz, 4H), 3.01 (s, 6H), 2.02-1.94 (m, 2H), 1.65-1.60 (m, 4H), 1.50-1.44 (m, 4H).

2,2′-(1,4-cis-cyclohexanediyl)diacetonitrile (16.3): ¹H NMR (600 MHz, Chloroform-d) δ 2.35 (dd, J=7.4, 1.4 Hz, 4H), 2.01-1.94 (m, 2H), 1.72-1.66 (m, 4H), 1.54-1.47 (m, 4H).

2,2′-(1,4-cis-cyclohexanediyl)diacetic acid (16.4): ¹H NMR (600 MHz, Methanol-d₄) δ 2.28 (d, J=7.5 Hz, 4H), 2.03-1.95 (m, 2H), 1.58 (ddq, J=13.4, 7.3, 3.8, 2.8 Hz, 4H), 1.44-1.38 (m, 4H).

2-((1,4-cis)-4-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethyl)cyclohexyl)acetic acid (16.5): ¹H NMR (600 MHz, Chloroform-d) δ 8.67 (s, 1H), 7.45 (d, J=7.8 Hz, 1H), 7.40 (d, J=8.2 Hz, 2H), 7.36 (d, J=8.3 Hz, 2H), 6.21 (d, J=8.6 Hz, 1H), 5.07 (p, J=7.0 Hz, 1H), 4.75 (t, J=7.9 Hz, 1H), 4.54 (d, J=8.7 Hz, 1H), 4.51 (s, 1H), 4.14 (d, J=11.4 Hz, 1H), 3.57 (dd, J=11.4, 3.6 Hz, 1H), 2.56 (ddd, J=12.6, 7.3, 4.8 Hz, 1H), 2.53 (s, 3H), 2.19-2.10 (m, 2H), 2.10-2.03 (m, 3H), 1.92 (t, J=13.5 Hz, 2H), 1.47 (d, J=7.0 Hz, 3H), 1.45-1.38 (m, 3H), 1.04 (s, 9H).

(2S,4R)-1-((2S)-2-(2-((1,4-cis)-4-(2-(4-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)piperazin-1-yl)-2-oxoethyl)cyclohexyl)acetamido)-3,3-dimethylbutanoyl)-4-hydroxy-N—((S)-1-(4-(4-Methylthiazol-5-yl)phenyl)ethyl)pyrrolidine-2-carboxamide (degrader #93): ¹H NMR (600 MHz, Chloroform-d) δ 8.67 (s, 1H), 8.32 (s, 1H), 8.11 (t, J=9.5 Hz, 1H), 7.69 (dd, J=13.8, 8.8 Hz, 2H), 7.43-7.34 (m, 7H), 7.30 (dd, J=14.2, 6.5 Hz, 4H), 7.05 (d, J=8.6 Hz, 1H), 6.98 (d, J=8.2 Hz, 2H), 6.76 (dd, J=8.7, 4.6 Hz, 2H), 6.61 (d, J=9.4 Hz, 1H), 6.21 (dd, J=14.2, 9.5 Hz, 1H), 5.09 (p, J=7.2 Hz, 1H), 4.74 (t, J=7.6 Hz, 1H), 4.64 (dd, J=14.8, 8.8 Hz, 1H), 4.51 (s, 1H), 4.17 (d, J=11.5 Hz, 1H), 3.91 (s, 1H), 3.65 (d, J=8.7 Hz, 4H), 3.58 (d, J=11.4 Hz, 2H), 3.43 (s, 2H), 3.23 (s, 4H), 3.11 (dt, J=13.9, 6.3 Hz, 2H), 3.02 (dd, J=13.8, 7.1 Hz, 1H), 2.89-2.74 (m, 2H), 2.62 (s, 1H), 2.51 (d, J=2.4 Hz, 3H), 2.34 (d, J=9.3 Hz, 6H), 2.26 (d, J=5.0 Hz, 5H), 2.14-2.05 (m, 3H), 1.70-1.58 (m, 8H), 1.54-1.49 (m, 5H), 1.47 (d, J=8.2 Hz, 5H), 1.35-1.27 (m, 7H), 1.25 (s, 3H), 1.05 (s, 10H), 0.96 (s, 3H).

### Example 53: Preparation of Degrader #94-95

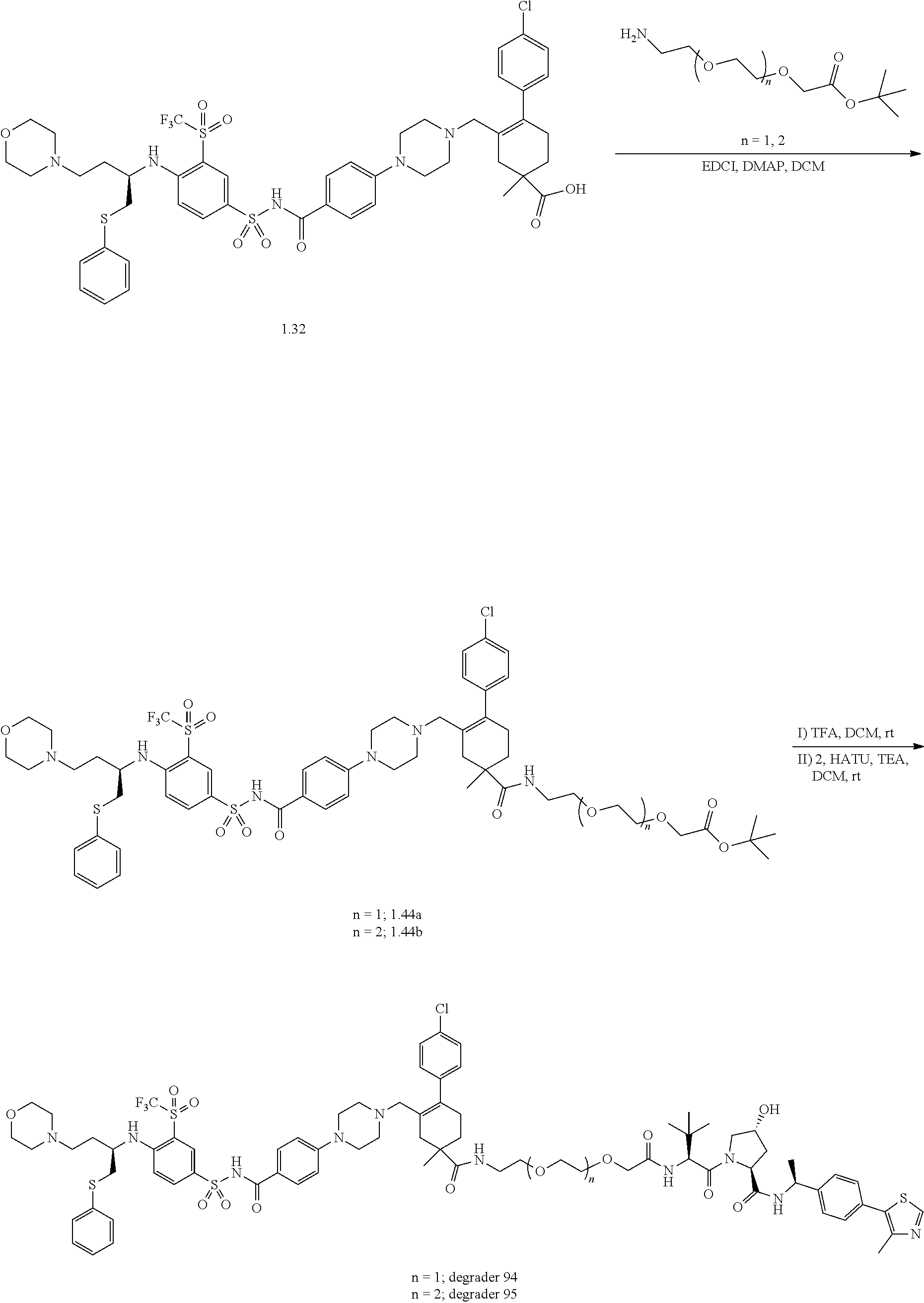

Synthesis of tert-butyl 2-(2-(2-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-carboxamido)ethoxy)ethoxy)acetate (1.44a): To a stirring solution of acid 1.32 (1 equiv.) in DCM was added appropriate amine (1 equiv.), EDCI (2 equiv.) and DMAP (2 equiv.). The reaction mixture was stirred for 6 h at room temperature. After consumption if the starting material the solvent was evaporated in reduced pressure and the crude was purified by flash chromatography to get the desired compound. ¹H NMR (600 MHz, Chloroform-d) δ 8.33 (s, 1H), 8.07 (d, 1H), 7.81 (br, 2H), 7.36 (d, J=7.5 Hz, 2H), 7.32-7.25 (m, 3H), 7.25-7.18 (m, 1H), 6.99 (d, J=8.0 Hz, 2H), 6.92 (br, 1H), 6.71 (d, J=8.5 Hz, 2H), 6.66 (br, 1H), 6.54 (d, J=9.3 Hz, 1H), 3.99 (s, 2H), 3.85 (br, 1H), 3.66 (d, J=7.2 Hz, 9H), 3.58 (br, 2H), 3.54-3.43 (m, 2H), 3.24 (br, 4H), 3.09 (dd, J=13.7, 4.7 Hz, 1H), 2.99 (dd, J=13.8, 7.4 Hz, 1H), 2.96-2.84 (m, 1H), 2.75 (d, J=17.7 Hz, 1H), 2.56-2.28 (m, 13H), 2.28-2.14 (m, 1H), 2.08 (br, 2H), 1.72-1.59 (m, 2H), 1.45 (s, 9H), 1.27 (s, 3H).

tert-butyl 1-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)-1-oxo-5,8,11-trioxa-2-azatridecan-13-oate (1.44b): Compound 1.44b was prepared following the same synthetic procedure as compound 1.44a was synthesized from acid 1.32. ¹H NMR (600 MHz, Chloroform-d) δ 8.34 (brs, 1H), 8.08 (br, 1H), 7.79 (br, 2H), 7.38 (br, 2H), 7.34-7.18 (m, 4H), 7.03-6.89 (m, 3H), 6.74 (br, 2H), 6.69 (br, 1H), 6.56 (br, 1H), 4.02 (s, 2H), 3.87 (br, 1H), 3.76-3.40 (m, 19H), 3.22 (br, 4H), 3.10 (d, J=13.4 Hz, 1H), 3.04-2.95 (m, 1H), 2.91 (d, J=12.2 Hz, 1H), 2.83 (d, J=12.6 Hz, 1H), 2.70 (d, J=17.6 Hz, 1H), 2.56-2.29 (m, 11H), 2.28-1.99 (m, 3H), 1.77-1.58 (m, 2H), 1.46 (s, 9H), 1.26 (s, 3H).

General synthetic procedure for the synthesis of degraders #94-95: To the stirring solution of the compound 1.44a or 1.44b (1 equiv.) in DCM was added TFA (20 equiv.) and the reaction mixture was stirred overnight. Upon completion of the reaction the volatiles were removed in the reduced pressure and the crude was used in the next step without further purification. To the stirring solution of the crude in DCM from each of the reaction was added amine 2 (1 equiv.), HATU (1.2 equiv.), TEA (15 equiv.). The reaction was stirred until the consumption of the starting material. The solvent was removed under reduced pressure and the crude product was purified by flash column chromatography (DCM/MeOH/TEA=96:5:1). The purified compound was dissolved in DCM and washed with saturated aqueous NH₄Cl. The organic portion was dried over Na₂SO₄, filtered, and DCM was evaporated under reduced pressure to afford the corresponding degraders 94 and 95.

(2S,4R)-1-((12S)-12-(tert-butyl)-1-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)-1,10-dioxo-5,8-dioxa-2,11-diazatridecan-13-oyl)-4-hydroxy-N—((S)-1-(4-(4-methylthiazol-5-yl)phenyl)ethyl)pyrrolidine-2-carboxamide (degrader #94): ¹H NMR (600 MHz, Chloroform-d) δ 8.69 (d, J=1.6 Hz, 1H), 8.34 (dd, J=8.2, 2.3 Hz, 1H), 8.11 (dtd, J=9.2, 6.8, 2.2 Hz, 1H), 7.71-7.62 (m, 2H), 7.43-7.35 (m, 6H), 7.37-7.24 (m, 5H), 7.04 (dd, J=8.7, 5.1 Hz, 1H), 7.01-6.94 (m, 2H), 6.73 (t, J=8.4 Hz, 3H), 6.63 (dd, J=9.6, 2.0 Hz, 1H), 5.14-5.06 (m, 1H), 4.78-4.61 (m, 2H), 4.54 (d, J=4.5 Hz, 1H), 4.11-4.02 (m, 1H), 3.95-3.87 (m, 1H), 3.87-3.71 (m, 2H), 3.71-3.50 (m, 12H), 3.49-3.35 (m, 1H), 3.32-3.17 (m, 4H), 3.12 (dd, J=13.9, 5.0 Hz, 1H), 3.03 (dd, 1H), 3.01-2.82 (m, 2H), 2.74 (d, J=17.5 Hz, 1H), 2.54-2.48 (m, 5H), 2.48-2.35 (m, 7H), 2.35-2.29 (m, 2H), 2.28-2.17 (m, 3H), 2.17-2.07 (m, 3H), 1.74-1.58 (m, 2H), 1.49 (dd, J=7.0, 1.6 Hz, 3H), 1.34-1.18 (m, 6H), 1.07 (d, J=4.5 Hz, 9H).

(2S,4R)-1-((15S)-15-(tert-butyl)-1-(4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)-1,13-dioxo-5,8,11-trioxa-2,14-diazahexadecan-16-oyl)-4-hydroxy-N—((S)-1-(4-(4-methylthiazol-5-yl)phenyl)ethyl)pyrrolidine-2-carboxamide (degrader #95): ¹H NMR (600 MHz, Chloroform-d) δ 8.69 (s, 1H), 8.34 (t, J=2.1 Hz, 1H), 8.12 (dd, J=9.3, 2.2 Hz, 1H), 7.74-7.64 (m, 2H), 7.43-7.36 (m, 7H), 7.37-7.24 (m, 5H), 7.05 (d, J=8.6 Hz, 1H), 6.98 (d, 2H), 6.77 (dd, J=9.2, 2.7 Hz, 2H), 6.63 (d, J=9.4 Hz, 1H), 6.60-6.51 (m, 1H), 5.10 (p, J=7.1 Hz, 1H), 4.72 (q, J=7.8 Hz, 1H), 4.64 (d, 1H), 4.52 (br, 1H), 4.10 (d, J=11.4 Hz, 1H), 3.97-3.84 (m, 2H), 3.73-3.39 (m, 17H), 3.26 (br, 4H), 3.12 (dd, J=13.9, 5.1 Hz, 1H), 3.04 (dd, J=13.9, 7.2 Hz, 1H), 2.93 (d, J=12.7 Hz, 1H), 2.86-2.76 (m, 1H), 2.70 (dd, J=17.6, 5.4 Hz, 1H), 2.51 (d, J=2.1 Hz, 3H), 2.50-2.28 (m, 9H), 2.28-2.21 (m, 2H), 2.21-2.16 (m, 2H), 2.16-2.07 (m, 4H), 1.78-1.56 (m, 2H), 1.49 (dd, J=6.9, 1.2 Hz, 3H), 1.33-1.18 (m, 6H), 1.07 (d, J=2.1 Hz, 9H).

### Example 54: Preparation of Degrader #96-97

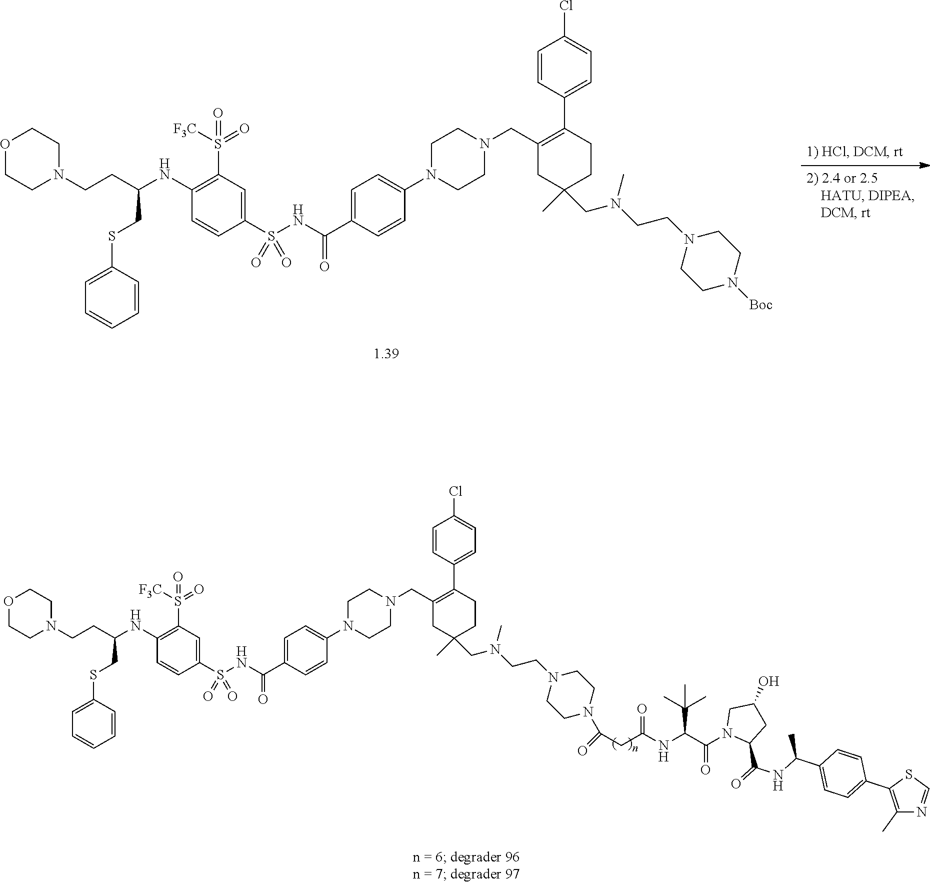

General synthesis of degraders #96-97: To the stirring solution of the compound 1.39 (1 equiv.) in DCM was added HCl solution in dioxane (10 equiv.) and the reaction mixture was stirred overnight. Upon completion of the reaction the volatiles were removed in the reduced pressure and the crude was used in the next step without further purification. To a stirring solution of crude in DCM added amine 2.4 or 2.5 (1 equiv.), HATU (1.2 equiv.), DIPEA (15 equiv.). The reaction was stirred until the consumption of the starting material. The solvent was removed under reduced pressure and the crude product was purified by flash column chromatography (DCM/MeOH/TEA=96:8:1). The purified compound was mixed dissolved in DCM and washed with saturated aqueous NH₄Cl. The organic portion was dried over Na₂SO₄, filtered, and DCM was evaporated under reduced pressure to afford the corresponding degraders 96 and 97.

(2S,4R)-1-((2S)-2-(8-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)-8-oxooctanamido)-3,3-dimethylbutanoyl)-4-hydroxy-N—((S)-1-(4-(4-methylthiazol-5-yl)phenyl)ethyl)pyrrolidine-2-carboxamide (degrader #96): ¹H NMR (600 MHz, Chloroform-d) δ 8.68 (s, 1H), 8.34 (d, J=2.2 Hz, 1H), 8.11 (dd, J=9.2, 2.2 Hz, 1H), 7.74 (dd, J=9.0, 2.1 Hz, 2H), 7.44-7.34 (m, 7H), 7.35-7.21 (m, 5H), 7.01 (dd, J=16.2, 8.4 Hz, 3H), 6.76 (d, J=8.7 Hz, 2H), 6.61 (d, J=9.4 Hz, 1H), 6.49 (dd, J=9.1, 5.5 Hz, 1H), 5.09 (p, J=7.3 Hz, 1H), 4.71 (td, J=8.1, 2.0 Hz, 1H), 4.65 (dd, J=9.0, 2.1 Hz, 1H), 4.50 (s, 1H), 4.11 (d, J=11.4 Hz, 1H), 3.95-3.83 (m, 1H), 3.74-3.62 (m, 4H), 3.62-3.50 (m, 3H), 3.48-3.37 (m, 2H), 3.24 (br, 4H), 3.12 (dd, J=13.8, 5.0 Hz, 1H), 3.03 (dd, J=13.6, 7.0 Hz, 1H), 2.86 (br, 2H), 2.67 (br, 2H), 2.60-2.17 (m, 29H), 2.17-2.06 (m, 3H), 2.04-1.92 (m, 1H), 1.75-1.65 (m, 2H), 1.65-1.50 (m, 4H), 1.51-1.40 (m, 4H), 1.38-1.13 (m, 9H), 1.06 (s, 9H), 0.97 (s, 3H).

(2S,4R)-1-((2S)-2-(9-(4-(2-(((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)(methyl)amino)ethyl)piperazin-1-yl)-9-oxononanamido)-3,3-dimethylbutanoyl)-4-hydroxy-N—((S)-1-(4-(4-methylthiazol-5-yl)phenyl)ethyl)pyrrolidine-2-carboxamide (degrader #97): ¹H NMR (600 MHz, Chloroform-d) δ 8.66 (s, 1H), 8.32 (d, J=2.2 Hz, 1H), 8.07 (d, 1H), 7.71 (dd, J=9.0, 3.2 Hz, 2H), 7.45-7.39 (m, 1H), 7.39-7.32 (m, 6H), 7.32-7.19 (m, 5H), 7.04-6.91 (m, 3H), 6.67 (br, 2H), 6.60 (d, J=9.4 Hz, 1H), 6.44 (d, J=9.1 Hz, 1H), 5.06 (p, J=7.1 Hz, 1H), 4.67 (t, 1H), 4.62 (d, J=9.2 Hz, 1H), 4.46 (br, 1H), 4.05 (d, J=11.2 Hz, 1H), 3.92-3.83 (m, 1H), 3.71-3.60 (m, 4H), 3.60-3.48 (m, 3H), 3.42 (br, 2H), 3.26 (br, 4H), 3.10 (dd, J=13.8, 5.0 Hz, 1H), 3.01 (dd, J=13.8, 7.2 Hz, 1H), 2.82-2.56 (m, 6H), 2.56-2.47 (m, 5H), 2.44 (br, 6H), 2.40-2.26 (m, 10H), 2.26-2.14 (m, 6H), 2.14-2.01 (m, 3H), 1.93-1.47 (m, 6H), 1.45 (d, J=6.9, 1.3 Hz, 3H), 1.32-1.14 (m, 12H), 1.03 (s, 12H).

### Example 55: Preparation of Degrader #98-100

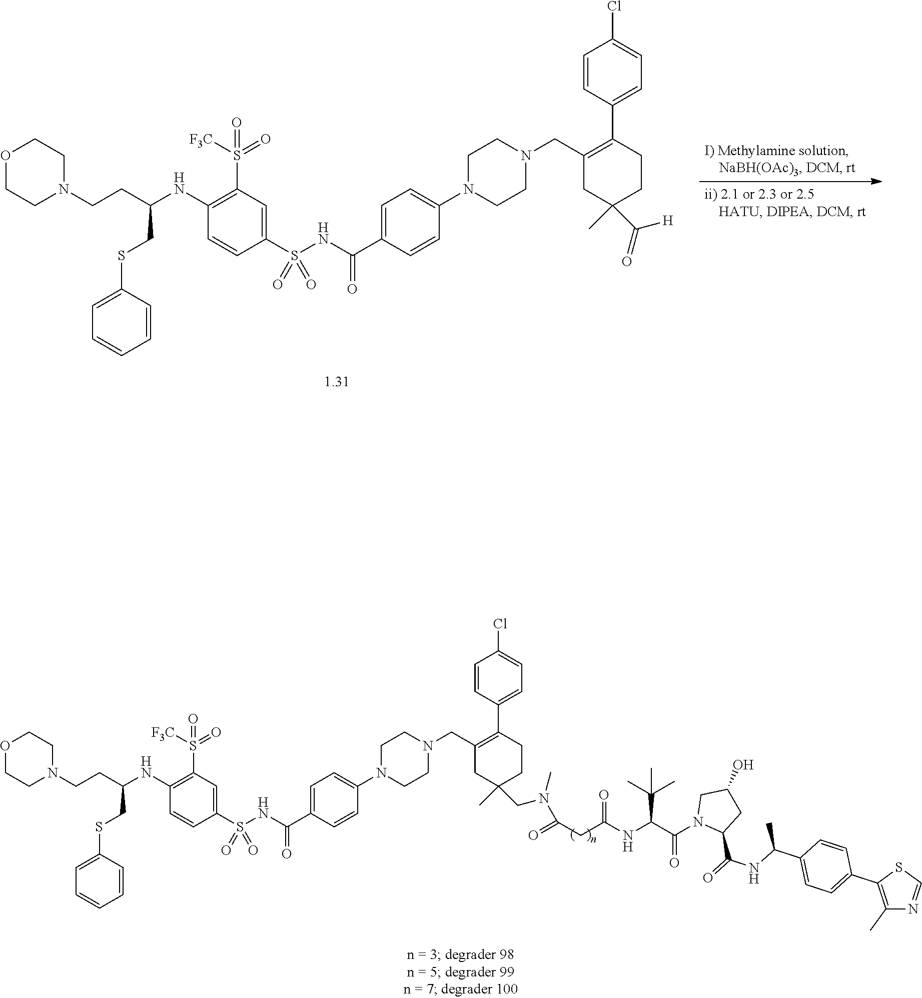

General synthesis of degraders #98-100: To a stirring solution of the aldehyde 1.31 (1 equiv.) was added methyl amine solution (5 equiv.) in THF and NaBH(OAc)₃ (3 equiv.). The above mixture was stirred until the consumption of the starting aldehyde. Once the reaction was complete, the mixture was further diluted with DCM and washed with saturated solution of NH₄Cl followed by the water and brine. The organic portion was dried over anhydrous MgSO₄, filtered, and then concentrated under reduced pressure. The crude was directly used in the next step.

To the stirring solution of the crude in DCM was added 1 equiv. of amine 2.1, 2.3, or 2.5 followed by the addition of HATU (1.2 equiv.) and TEA (15 equiv.). The reaction was stirred until the consumption of the starting material. The solvent was removed under reduced pressure and the crude product was purified by flash column chromatography (DCM/MeOH/TEA=96:5:1). The purified compound was dissolved in DCM and washed with saturated aqueous NH₄Cl.

The organic portion was dried over Na₂SO₄, filtered, and DCM was evaporated under reduced pressure to afford the corresponding degraders #98-100.

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N5-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N1-methylglutaramide (degrader #98): ¹H NMR (600 MHz, Chloroform-d) δ 8.70 (d, 1H), 8.34 (d, J=2.3 Hz, 1H), 8.12 (dd, J=9.4, 2.2 Hz, 1H), 7.68 (dd, J=9.2, 3.0 Hz, 2H), 7.53-7.44 (m, 1H), 7.43-7.34 (m, 6H), 7.35-7.22 (m, 5H), 7.06-6.95 (m, 3H), 6.80-6.71 (m, 2H), 6.61 (d, J=9.3 Hz, 1H), 5.09 (p, J=7.1 Hz, 1H), 4.75-4.64 (m, 1H), 4.55-4.50 (m, 1H), 4.48 (br, 1H), 4.15-4.06 (m, 1H), 3.95-3.85 (m, 1H), 3.73-3.60 (m, 4H), 3.56 (dd, J=11.6, 3.8 Hz, 1H), 3.37 (br, 2H), 3.31-3.20 (m, 4H), 3.20-3.10 (m, 1H), 3.09 (s, 3H), 3.06-2.98 (m, 2H), 2.93-2.79 (m, 2H), 2.50 (d, J=2.4 Hz, 3H), 2.48-2.21 (m, 12H), 2.18-1.90 (m, 5H), 1.74-1.63 (m, 3H), 1.63-1.51 (m, 2H), 1.48 (d, J=7.0, 1.5 Hz, 3H), 1.41-1.12 (m, 6H), 1.06 (s, 9H), 1.00 (s, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N7-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N1-methylheptanediamide (degrader #99): ¹H NMR (600 MHz, Chloroform-d) δ 8.69 (s, 1H), 8.33 (d, J=2.3 Hz, 1H), 8.11 (dd, J=9.3, 2.6 Hz, 1H), 7.71 (dd, J=8.6, 5.4 Hz, 2H), 7.45-7.34 (m, 7H), 7.34-7.22 (m, 5H), 7.05-6.94 (m, 3H), 6.81-6.73 (m, 2H), 6.61 (d, J=9.4, 2.7 Hz, 1H), 6.51-6.41 (m, 1H), 5.10 (p, J=7.1 Hz, 1H), 4.75-4.67 (m, 1H), 4.63 (dd, J=9.0, 1.9 Hz, 1H), 4.50 (s, 1H), 4.08 (d, J=11.3 Hz, 1H), 3.95-3.84 (m, 1H), 3.73-3.61 (m, 4H), 3.59 (dd, J=11.5, 3.4 Hz, 1H), 3.41-3.30 (m, 1H), 3.30-3.20 (m, 4H), 3.20-3.10 (m, 2H), 3.09 (s, 3H), 3.05-2.98 (m, 2H), 2.94-2.81 (m, 2H), 2.52 (s, 3H), 2.48-2.24 (m, 12H), 2.24-2.07 (m, 4H), 2.07-1.93 (m, 1H), 1.77-1.51 (m, 6H), 1.49 (d, J=6.9, 1.6 Hz, 3H), 1.39-1.30 (m, 3H), 1.30-1.17 (m, 5H), 1.04 (d, J=3.0 Hz, 9H), 1.00 (s, 3H).

N1-((4′-chloro-4-methyl-6-((4-(4-(((4-(((R)-4-morpholino-1-(phenylthio)butan-2-yl)amino)-3-((trifluoromethyl)sulfonyl)phenyl)sulfonyl)carbamoyl)phenyl)piperazin-1-yl)methyl)-2,3,4,5-tetrahydro-[1,1′-biphenyl]-4-yl)methyl)-N9-((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)-N1-methylnonanediamide (degrader #100): ¹H NMR (600 MHz, Chloroform-d) δ 8.69 (s, 1H), 8.33 (s, 1H), 8.13 (d, J=8.9 Hz, 1H), 7.80-7.65 (m, 2H), 7.48-7.35 (m, 7H), 7.35-7.21 (m, 5H), 7.07 (d, J=8.6 Hz, 1H), 7.00 (dd, J=10.4, 7.7 Hz, 2H), 6.79 (d, J=8.3 Hz, 2H), 6.63 (d, J=9.3 Hz, 1H), 6.42-6.26 (m, 1H), 5.11 (q, J=7.2 Hz, 1H), 4.82-4.64 (m, 2H), 4.51 (br, 1H), 4.22-4.08 (m, 1H), 3.92 (br, 1H), 3.76-3.63 (m, 4H), 3.63-3.52 (m, 1H), 3.42-3.29 (m, 1H), 3.29-3.16 (m, 4H), 3.16-3.07 (m, 4H), 3.04 (dd, J=14.6, 6.0 Hz, 2H), 2.92-2.70 (m, 2H), 2.53 (s, 3H), 2.50-2.23 (m, 12H), 2.23-2.06 (m, 4H), 2.06-1.92 (m, 1H), 1.80-1.66 (m, 2H), 1.66-1.54 (m, 4H), 1.54-1.46 (m, 4H), 1.36-1.09 (m, 12H), 1.07 (s, 9H), 1.02 (s, 3H).

### Example 56: Preparation of Degrader #101

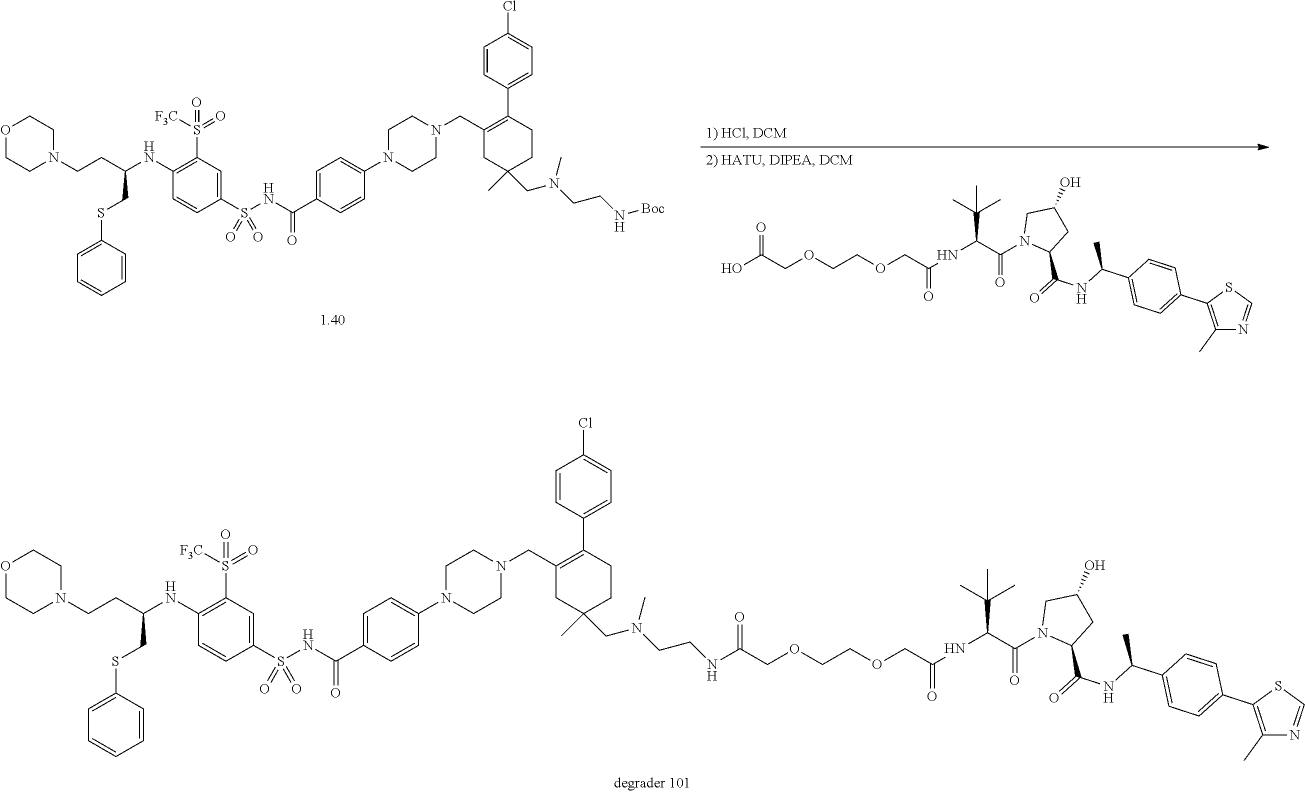 [SMILES: CN(CCNC(=O)OC(C)(C)C)CC1(C)CCC(c2ccc(Cl)cc2)=C(CN2CCN(c3ccc(C(=O)NS(=O)(=O)c4ccc(N[C@H](CCN5CCOCC5)CSc5ccccc5)c(S(=O)(=O)C(F)(F)F)c4)cc3)CC2)C1 ; Cc1ncsc1-c1ccc([C@H](C)NC(=O)[C@@H]2C[C@@H](O)CN2C(=O)[C@@H](NC(=O)COCCOCC(=O)O)C(C)(C)C)cc1 ; Cc1ncsc1-c1ccc([C@H](C)NC(=O)[C@@H]2C[C@@H](O)CN2C(=O)[C@@H](NC(=O)COCCOCC(=O)NCCN(C)CC2(C)CCC(c3ccc(Cl)cc3)=C(CN3CCN(c4ccc(C(=O)NS(=O)(=O)c5ccc(N[C@H](CCN6CCOCC6)CSc6ccccc6)c(S(=O)(=O)C(F)(F)F)c5)cc4)CC3)C2)C(C)(C)C)cc1]

To the stirring solution of the compound 1.40 (1 equiv.) in DCM was added HCl solution in dioxane (10 equiv.) and the reaction mixture was stirred overnight. Upon completion of the reaction the volatiles were removed in the reduced pressure and the crude was used in the next step without further purification. To a stirring solution of crude in DCM added 2-(2-(2-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-2-oxoethoxy)ethoxy)acetic acid (1 equiv.), HATU (1.2 equiv.), DIPEA (15 equiv.). The reaction was stirred until the consumption of the starting material. The solvent was removed under reduced pressure and the crude product was purified by flash column chromatography (DCM/MeOH/TEA=96:8:1). The purified compound was mixed dissolved in DCM and washed with saturated aqueous NH₄Cl. The organic portion was dried over Na₂SO₄, filtered, and DCM was evaporated under reduced pressure to afford the corresponding degrader #101. ¹H NMR (600 MHz, Chloroform-d) δ 8.68 (s, 1H), 8.33 (d, J=2.2 Hz, 1H), 8.10 (dd, J=9.3, 2.2 Hz, 1H), 7.70 (d, J=8.5 Hz, 2H), 7.47-7.42 (m, 1H), 7.42-7.33 (m, 7H), 7.33-7.20 (m, 5H), 7.02 (d, J=8.6 Hz, 1H), 6.98 (d, 2H), 6.76 (dd, 2H), 6.60 (d, J=9.4 Hz, 1H), 5.08 (p, J=7.1 Hz, 1H), 4.66 (td, J=8.1, 2.8 Hz, 1H), 4.62 (d, J=9.2 Hz, 1H), 4.49 (s, 1H), 4.06 (dd, J=15.6, 1.3 Hz, 1H), 4.03-3.99 (m, 2H), 3.98 (dd, J=11.0, 2.3 Hz, 1H), 3.93-3.85 (m, 2H), 3.76-3.59 (m, 9H), 3.45-3.33 (m, 2H), 3.23 (t, J=5.2 Hz, 4H), 3.11 (dd, J=13.9, 5.0 Hz, 1H), 3.03 (dd, J=13.9, 7.2 Hz, 1H), 2.83 (d, J=4.7 Hz, 2H), 2.67 (br, 2H), 2.54-2.48 (m, 3H), 2.48-2.19 (m, 17H), 2.17-2.06 (m, 2H), 1.98 (d, J=15.7 Hz, 1H), 1.85 (br, 2H), 1.73-1.63 (m, 1H), 1.61-1.54 (m, 1H), 1.54-1.50 (m, 1H), 1.49 (s, 3H), 1.34-1.17 (m, 3H), 1.04 (s, 9H), 0.99 (s, 3H).

### Example 57: Cell Viability Assay

Acute lymphoblastic leukemia cells (MOLT-4 and RS4; 11) or small cell lung cancer (NCI-H146 or simply H146) were incubated with increasing concentrations of Bcl-xL degraders for 48 h. Cell viability was measured by tetrazolium-based MTS assay. The IC₅₀ values of individual agents were calculated with GraphPad Prism and presented in Table 3, Table 4, and FIG. 7.

### Example 58: Protein Degradation Assays in MOLT-4 Cells and Human Platelets

MOLT-4 cells and human platelets were incubated with increasing concentrations of test compounds for 16 h. The cells were harvested and lysed in RIPA lysis buffer supplemented with protease and phosphatase inhibitor cocktails. An equal amount of protein (20 μg/lane) was resolved on a pre-cast 4-20% SDS-PAGE gel. Proteins were subsequently transferred to NOVEX PVDF membranes by electrophoresis. The membranes were blocked in blocking buffer (5% non-fat dry milk in TBS-T), and incubated with primary antibodies (at optimized concentrations) overnight at 4° C. After three washings in TBS-T, the membranes were incubated with an appropriate HRP-conjugated secondary antibody for 1 h at room temperature. After extensive washing for three times, the proteins of interest were detected with ECL western blotting detection reagents and recorded with autoradiography (Pierce Biotech, Rockford, Ill., USA). The primary antibodies for Bcl-xL (Cat #2762), Bcl-2 (Cat #2872), Mcl-1 (Cat #5453) and β-actin (Cat #4970) were purchased from Cell Signaling technology. The relative band intensity was measured using ImageJ software and normalized to β-actin. The DC₅₀ was calculated using GraphPad Prism. Representative data are presented in FIG. 8 and FIG. 9.

### Example 59: Elucidation of Downstream Apoptotic Mechanism by Degraders

MOLT-4 cells were incubated with increasing concentrations of degrader #5 or #83 for 24 h. At the end of incubation, cells were harvested for western blot analysis of cleaved and full length caspase-3 and poly (ADP) ribose polymerase (PARP). The antibodies for cleaved caspase-3 (Cat #9661) and PARP (Cat #9532) were purchased from Cell Signaling Technology. Representative data are presented in FIG. 10.

### Example 60: Ternary Complex Assay

To detect ternary complex formation induced by the compounds, AlphaLISA assay can be used to measure luminescence signals arisen from proximity of Bcl-xL bounded acceptor beads and VHL- or CRBN-bounded donor beads. Briefly, to a 96-well PCR plate, 10 μL of 20 nM 6-His tagged Bcl-xL protein can be mixed with 10 μL of 20 nM GST-tagged VHL complex protein and 10 μL of serially diluted testing compounds. After incubating at room temperature for 30 min, 5 μL of 160 μg/mL Glutathione donor beads (PerkinElmer) can be added and the mixture can be incubated at dark for 15 min. 5 μL of 160 μg/mL of anti-His acceptor beads can be added lastly and the mixture can be incubated for an additional 45 min before being transferred to two adjacent wells (17 μL each) of 384-well white OptiPlate (PerkinElmer). The luminescence signals can be detected on a Biotek's Synergy Neo2 multi-mode plate reader installed with an AphaScreen filter cube. All reagents can be diluted in an assay buffer of 25 mM HEPES, pH 7.5, 100 mM NaCl, 0.1% BSA, and 0.005% tween 20 prior incubation. Representative data are presented in FIG. 11.

### Results

### Compounds of the Invention Reduce the On-Target Toxicity (Thrombocytopenia) Relative to ABT-263

FIG. 7 depict the inhibitory effects of degrader #5, degrader #41 and degrader #42 (the chiral pure diastereomers of degrader #5), and ABT-263 on MOLT-4, RS4; 11, NCI-H146 cells, and human platelets. One diastereomer, degrader #42, was more potent than the other diastereomer, degrader #41, in all assays. Degrader #42 was more potent in killing MOLT-4 and RS4; 11 cells and equally potent in killing NCI-H146 cells (anti-cancer effect) when compared with ABT-263, but demonstrated substantially less effects on human platelets, as summarized below in Table 4.

TABLE 4

| | Platelets | MOLT-4 | NCI-H146 | RS4; 11 |
|---|---|---|---|---|
| | IC₅₀ | IC₅₀ | IC₅₀ | IC₅₀ |
| Compound | (μM, 48 h) | (nM, 48 h) | (nM, 48 h) | (nM, 48 h) |
| ABT-263 | 0.50 | 201 | 36 | 35 |
| degrader #5 | 3.50 | 31 | 88 | 22.2 |
| degrader #41 | 10.0 | 154 | 314 | 497 |
| degrader #42 | 1.09 | 15.2 | 45 | 9.9 |

### Compounds of the Invention Dose-Dependently Induce Bcl-xL Degradation

in MOLT-4 Cells but not in Human Platelets Degraders #5, #41, and #42 dose-dependently induced the degradation of Bcl-xL in MOLT-4 cells with DC₅₀ (concentration with 50% degradation) values of 21.5 nM, 100.5 nM, and 11.5 nM, respectively (FIG. 8). Degrader #5 did not affect Bcl-xL levels in human platelets (FIG. 9).

### Compounds of the Invention Induce Apoptotic Response in MOLT-4 Cells

Degraders #5 and #83 induced cleavage of caspase-3 and PARP in MOLT-4 cells after 16 h treatment (FIG. 10).

### Compounds of the Invention Form Ternary Complexes with VCB-Complex and Bcl-xL

Degraders #83, #84, and #85 formed ternary complexes with the VHL E3 ligase complex and Bcl-xL while their Bcl-xL binding portion (Bcl-xL ligand) did not (FIG. 11).

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

What is claimed:

1. A compound of Formula (I), or a pharmaceutically acceptable salt thereof:
Y-L₂-R-L₁-Y₂ Formula (I);
wherein L₁ is independently 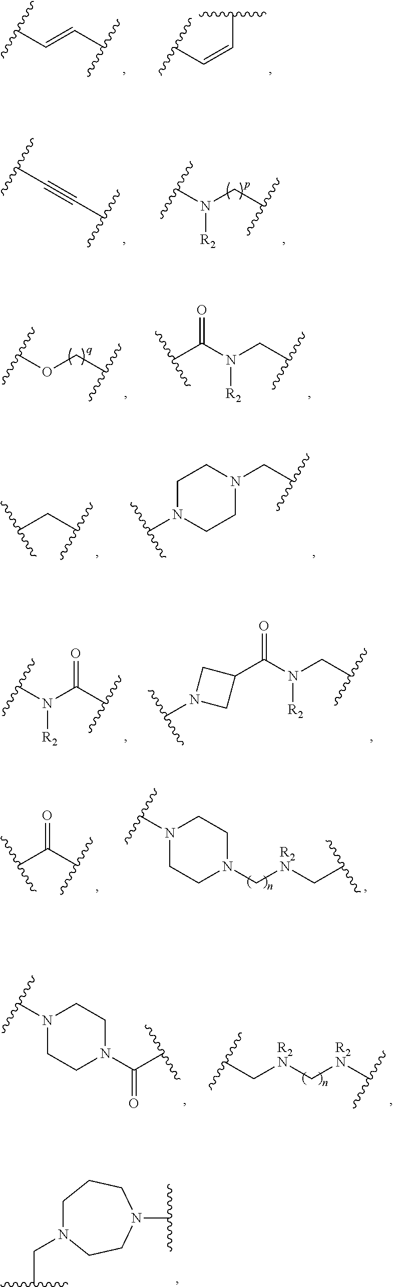 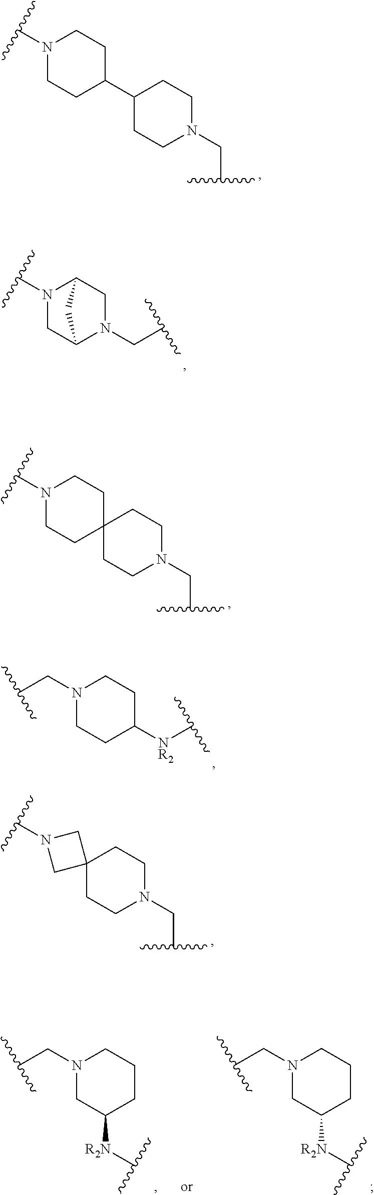
R is independently 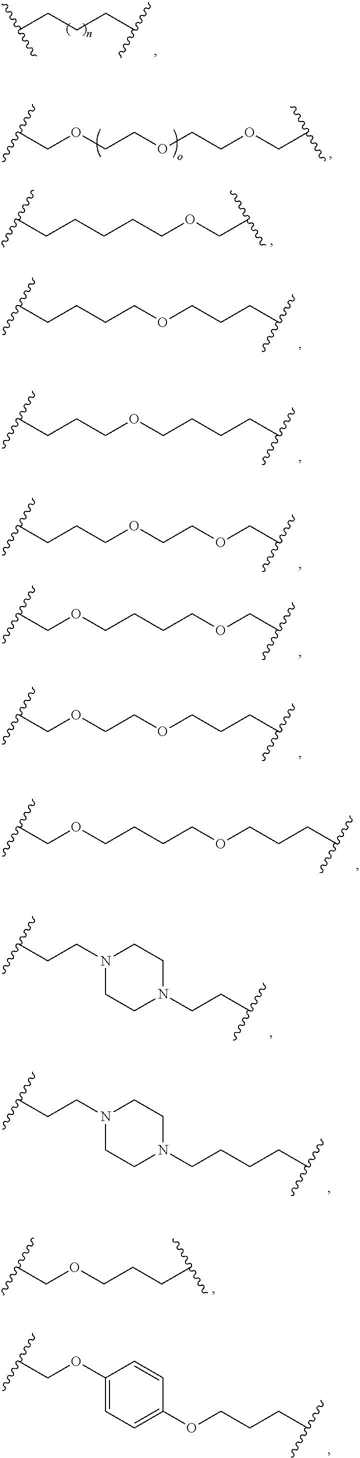 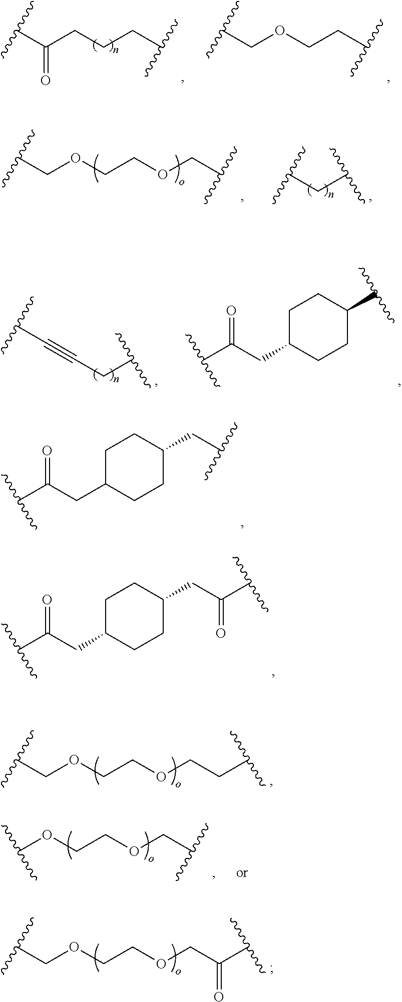
L₂ is independently 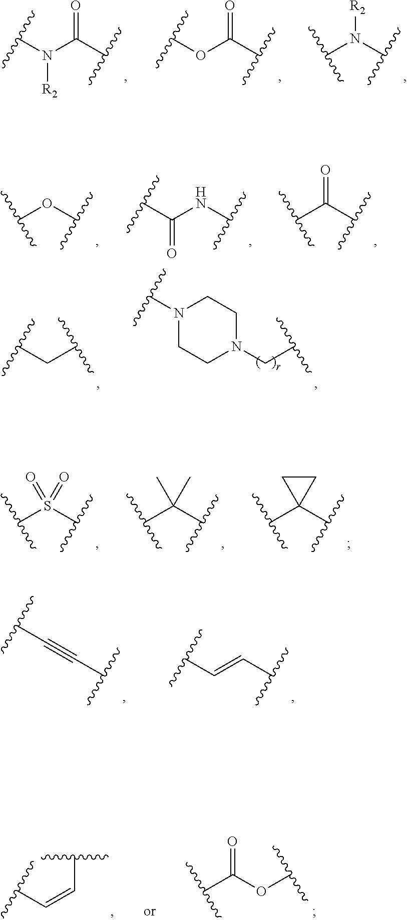
Y is independently 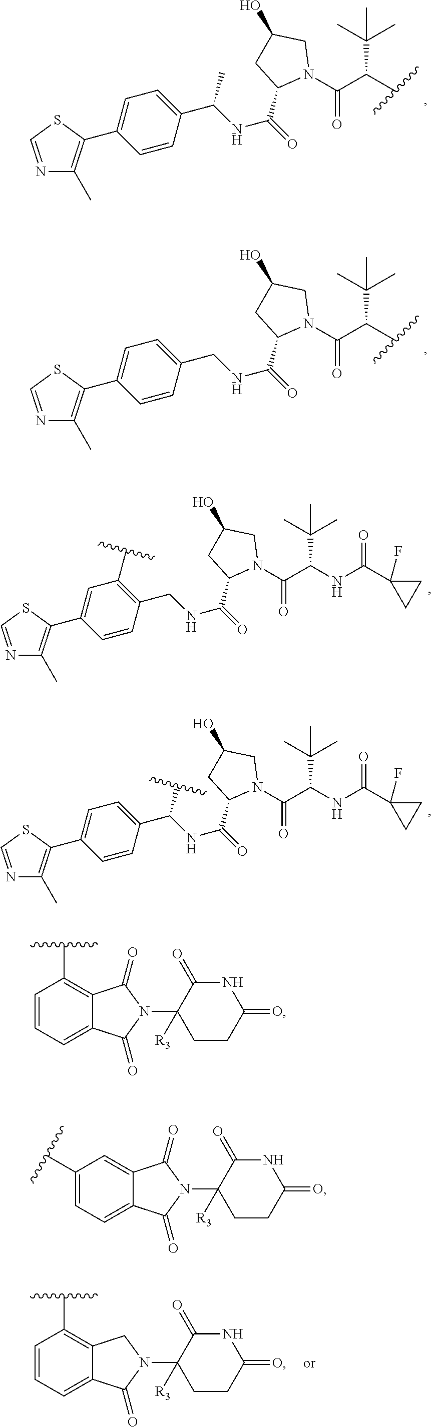 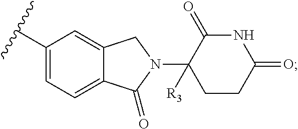
Y₂ is independently 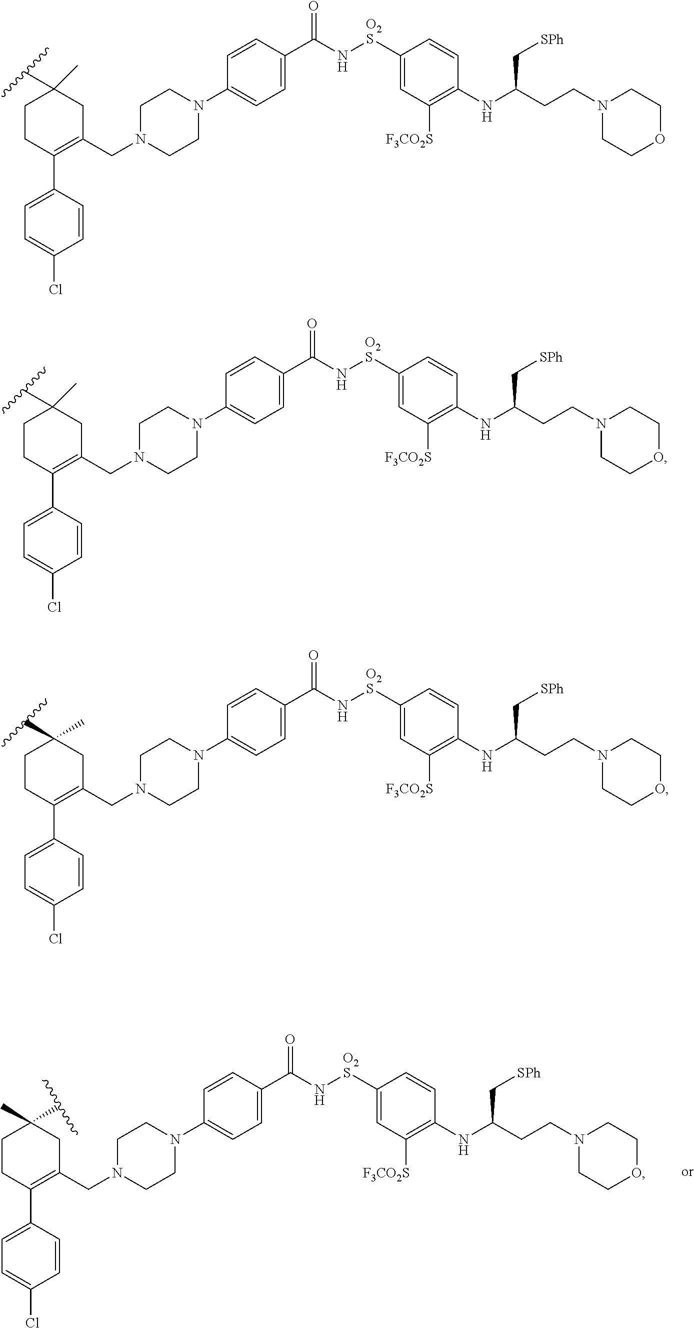 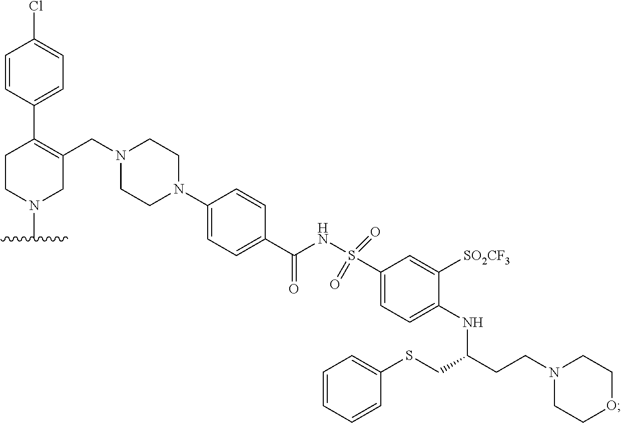
each R₂ is independently H, optionally substituted alkyl, or optionally substituted cycloalkyl;
each R₃ is independently H, D, CH₃, or F; and
each n, o, p, and q is independently 0-10, inclusive.

2. The compound of claim 1, or a pharmaceutically acceptable salt thereof, wherein R is

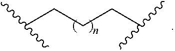

3. The compound of claim 1 or 2, or a pharmaceutically acceptable salt thereof, wherein n is 3-8, inclusive.

4. The compound of claim 1, or a pharmaceutically acceptable salt thereof, wherein L₂ is independently

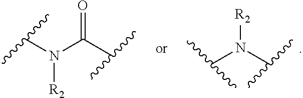

5. The compound of claim 1, or a pharmaceutically acceptable salt thereof, wherein L₂ is independently 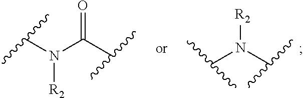
and R is

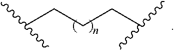

6. The compound of claim 1, or a pharmaceutically acceptable salt thereof, wherein L₂ is independently 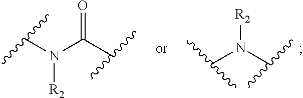
R is 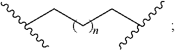
and n is 3-8, inclusive.

7. The compound of claim 1, or a pharmaceutically acceptable salt thereof, wherein L₁ is independently

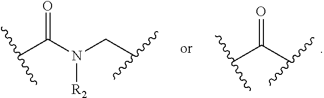

8. The compound of claim 1, or a pharmaceutically acceptable salt thereof, wherein L₁ is independently 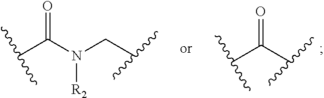
and R is

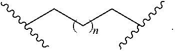

9. The compound of claim 1, or a pharmaceutically acceptable salt thereof, wherein L₁ is independently 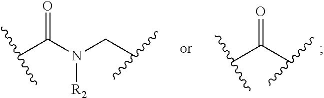
R is 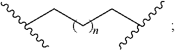
and n is 3-8, inclusive.

10. The compound of claim 1, or a pharmaceutically acceptable salt thereof, wherein L₂ is independently 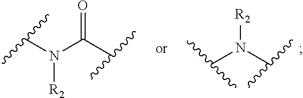
and L₁ is independently

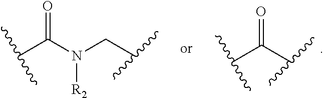

11. The compound of claim 1, or a pharmaceutically acceptable salt thereof, wherein L₂ is independently 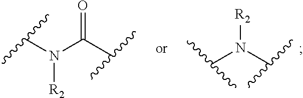
L₁ is independently 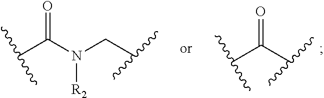
and R is

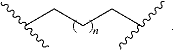

12. The compound of claim 1, or a pharmaceutically acceptable salt thereof, wherein L₂ is independently 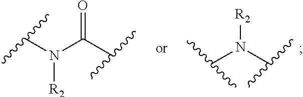
L₁ is independently 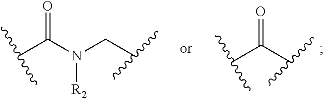
R is 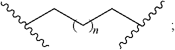
and n is 3-8, inclusive.

13. The compound of claim 1, or a pharmaceutically acceptable salt thereof, wherein Y is

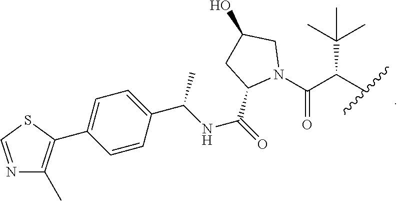 [SMILES: *[C@H](C(=O)N1C[C@H](O)C[C@H]1C(=O)N[C@@H](C)c1ccc(-c2scnc2C)cc1)C(C)(C)C]

14. The compound of claim 1, or a pharmaceutically acceptable salt thereof, wherein Y is 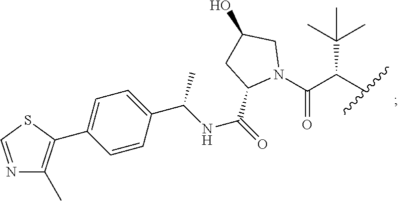 [SMILES: *[C@H](C(=O)N1C[C@H](O)C[C@H]1C(=O)N[C@@H](C)c1ccc(-c2scnc2C)cc1)C(C)(C)C]
and L₂ is

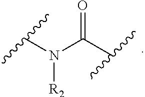

15. The compound of claim 1, or a pharmaceutically acceptable salt thereof, wherein Y is 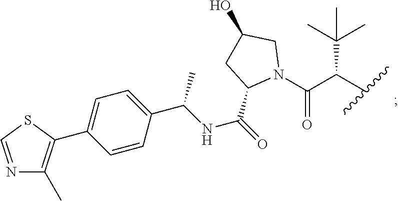 [SMILES: *[C@H](C(=O)N1C[C@H](O)C[C@H]1C(=O)N[C@@H](C)c1ccc(-c2scnc2C)cc1)C(C)(C)C]
L₂ is 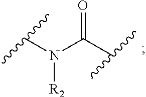
and R is

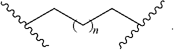

16. The compound of claim 1, or a pharmaceutically acceptable salt thereof, wherein Y is 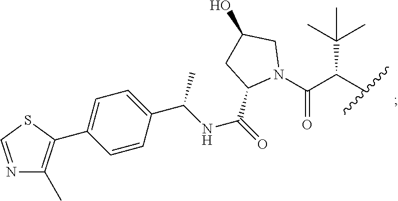 [SMILES: *[C@H](C(=O)N1C[C@H](O)C[C@H]1C(=O)N[C@@H](C)c1ccc(-c2scnc2C)cc1)C(C)(C)C]
L₂ is 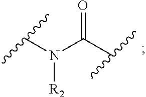
R is 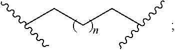
and n is 3-8, inclusive.

17. The compound of claim 1, or a pharmaceutically acceptable salt thereof, wherein Y is 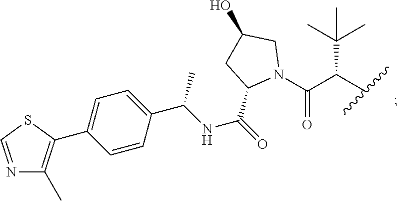 [SMILES: *[C@H](C(=O)N1C[C@H](O)C[C@H]1C(=O)N[C@@H](C)c1ccc(-c2scnc2C)cc1)C(C)(C)C]
L₂ is 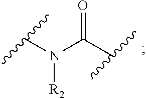
and L₁ is independently

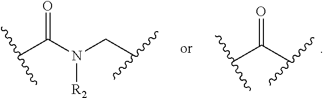

18. The compound of claim 1, or a pharmaceutically acceptable salt thereof, wherein Y is 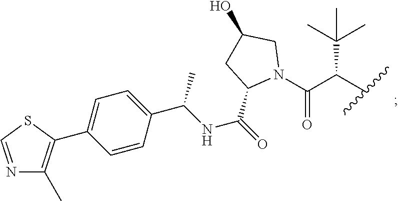 [SMILES: *[C@H](C(=O)N1C[C@H](O)C[C@H]1C(=O)N[C@@H](C)c1ccc(-c2scnc2C)cc1)C(C)(C)C]
L₂ is 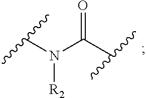
L₁ is independently 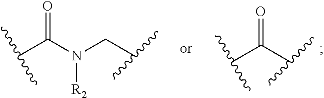
and R is

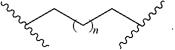

19. The compound of claim 1, or a pharmaceutically acceptable salt thereof, wherein Y is 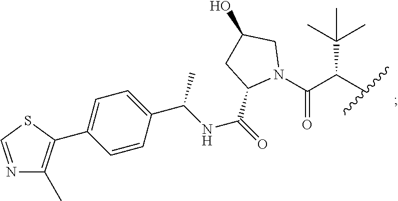 [SMILES: *[C@H](C(=O)N1C[C@H](O)C[C@H]1C(=O)N[C@@H](C)c1ccc(-c2scnc2C)cc1)C(C)(C)C]
L₂ is 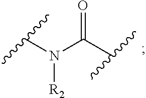
L₁ is independently 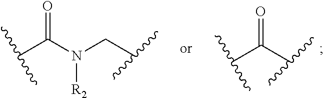
R is 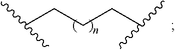
and n is 3-8, inclusive.

20. The compound of claim 1, or a pharmaceutically acceptable salt thereof, wherein Y₂ is

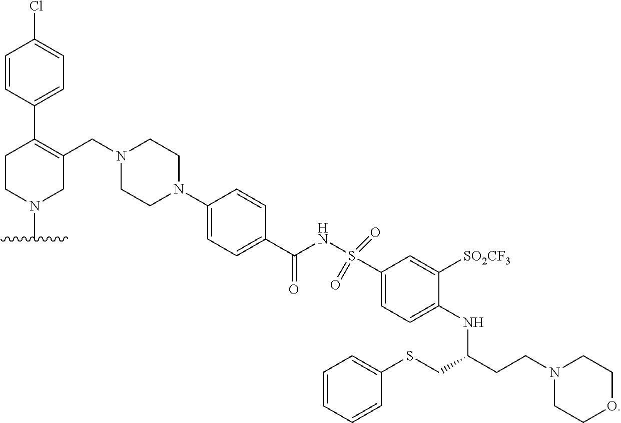

21. The compound of claim 1, or a pharmaceutically acceptable salt thereof, wherein Y₂ is 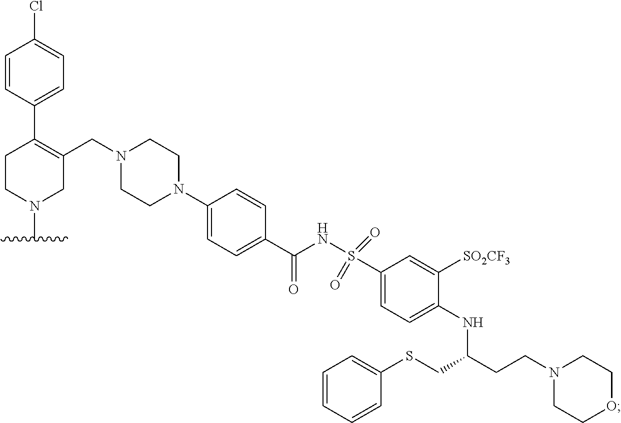
and L₂ is

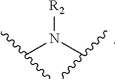

22. The compound of claim 1, or a pharmaceutically acceptable salt thereof, wherein Y₂ is 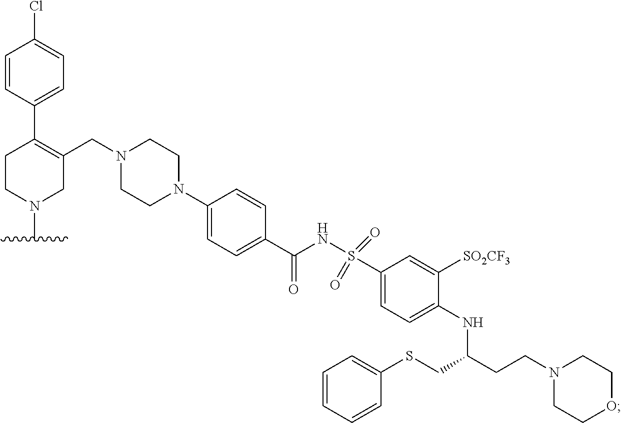
L₂ is 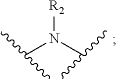
and R is

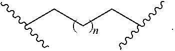

23. The compound of claim 1, or a pharmaceutically acceptable salt thereof, wherein Y₂ is 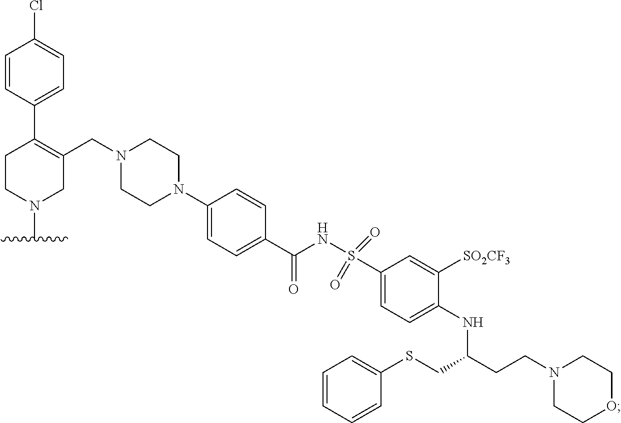
L₂ is 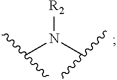
R is 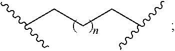
and n is 3-8, inclusive.

24. The compound of claim 1, or a pharmaceutically acceptable salt thereof, wherein Y₂ is 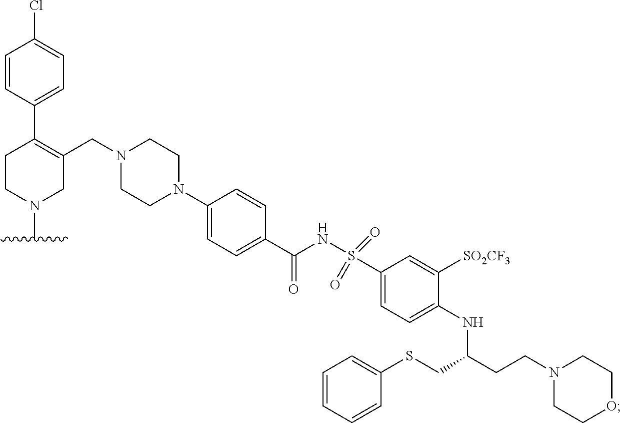
L₂ is 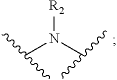
and L₁ is independently

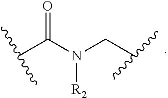

25. The compound of claim 1, or a pharmaceutically acceptable salt thereof, wherein Y₂ is 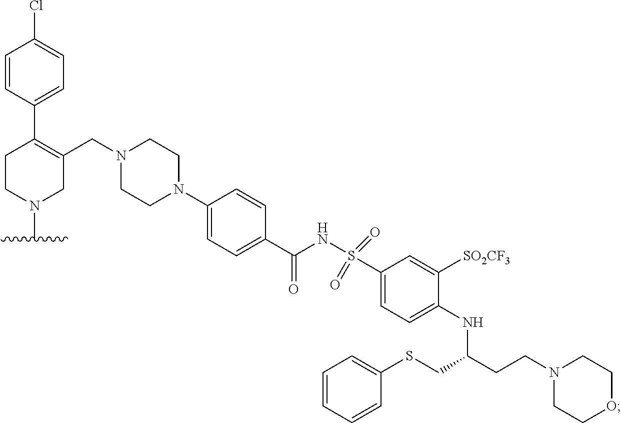
L₂ is 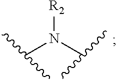
L₁ is independently 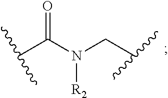
and R is

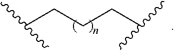

26. The compound of claim 1, or a pharmaceutically acceptable salt thereof, wherein Y₂ is 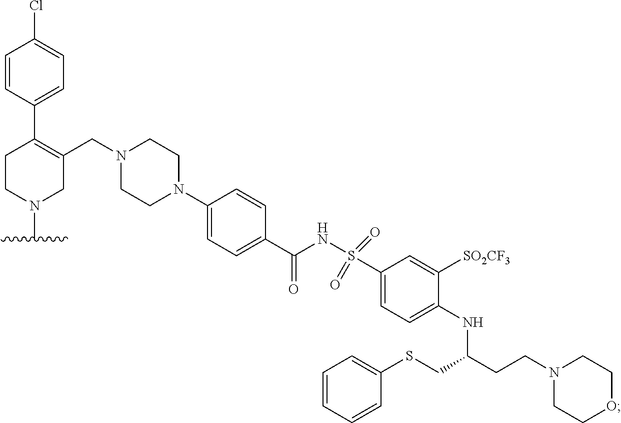
L₂ is 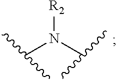
L₁ is independently 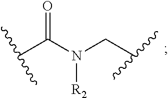
R is 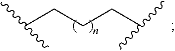
and n is 3-8, inclusive.

27. The compound of claim 1, or a pharmaceutically acceptable salt thereof, wherein Y is 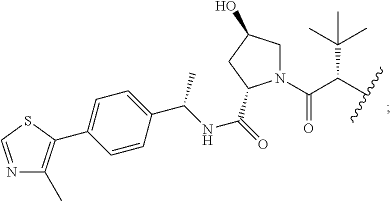 [SMILES: *[C@H](C(=O)N1C[C@H](O)C[C@H]1C(=O)N[C@@H](C)c1ccc(-c2scnc2C)cc1)C(C)(C)C]
Y₂ is 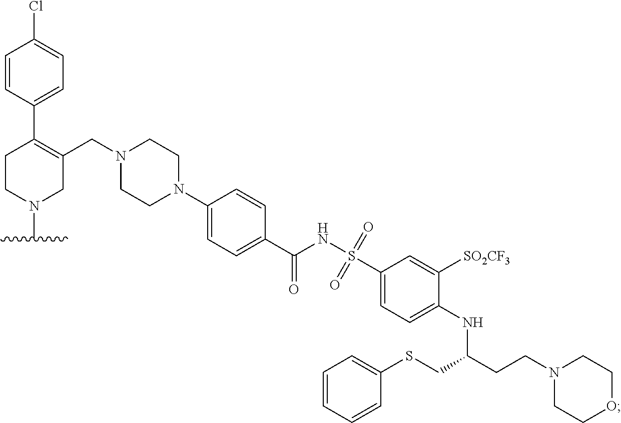
and L₂ is

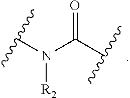

28. The compound of claim 1, or a pharmaceutically acceptable salt thereof, wherein
Y is 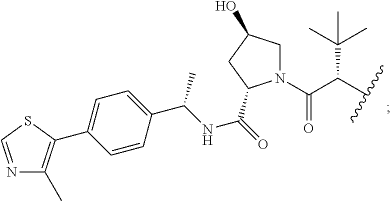 [SMILES: *[C@H](C(=O)N1C[C@H](O)C[C@H]1C(=O)N[C@@H](C)c1ccc(-c2scnc2C)cc1)C(C)(C)C]
Y₂ is 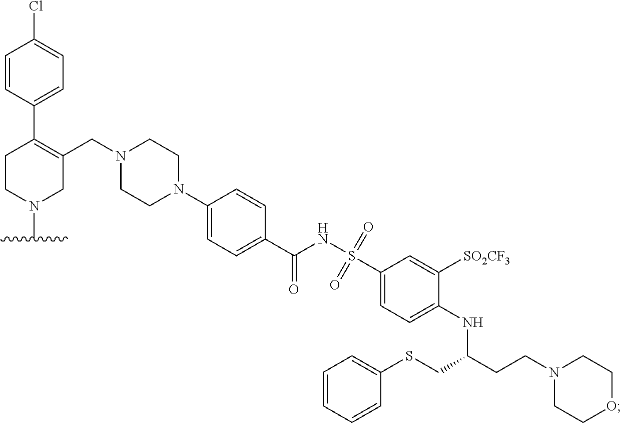
L₂ is 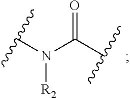
and R is

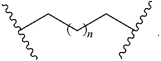

29. The compound of claim 1, or a pharmaceutically acceptable salt thereof, wherein Y is 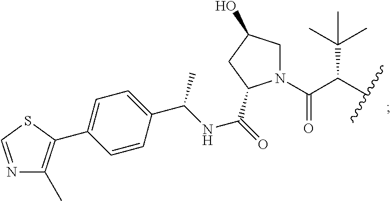 [SMILES: *[C@H](C(=O)N1C[C@H](O)C[C@H]1C(=O)N[C@@H](C)c1ccc(-c2scnc2C)cc1)C(C)(C)C]
Y₂ is 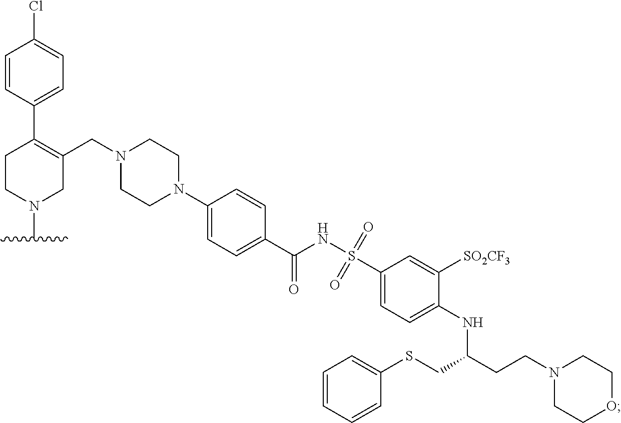
L₂ is 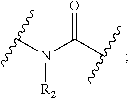
R is 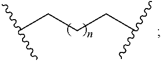
and n is 3-8, inclusive.

30. The compound of claim 1, or a pharmaceutically acceptable salt thereof, wherein Y is 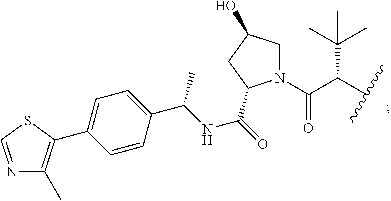 [SMILES: *[C@H](C(=O)N1C[C@H](O)C[C@H]1C(=O)N[C@@H](C)c1ccc(-c2scnc2C)cc1)C(C)(C)C]
Y₂ is 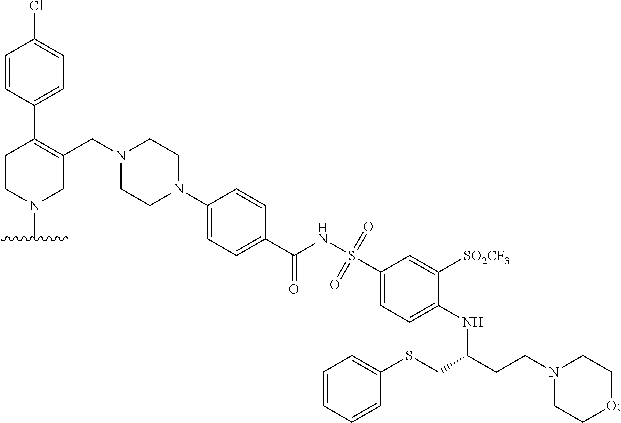
L₂ is 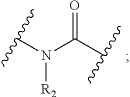
and L₁ is independently

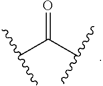 [SMILES: *C(*)=O]

31. The compound of claim 1, or a pharmaceutically acceptable salt thereof, wherein Y is 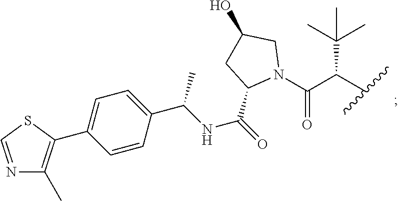 [SMILES: *[C@H](C(=O)N1C[C@H](O)C[C@H]1C(=O)N[C@@H](C)c1ccc(-c2scnc2C)cc1)C(C)(C)C]
Y₂ is 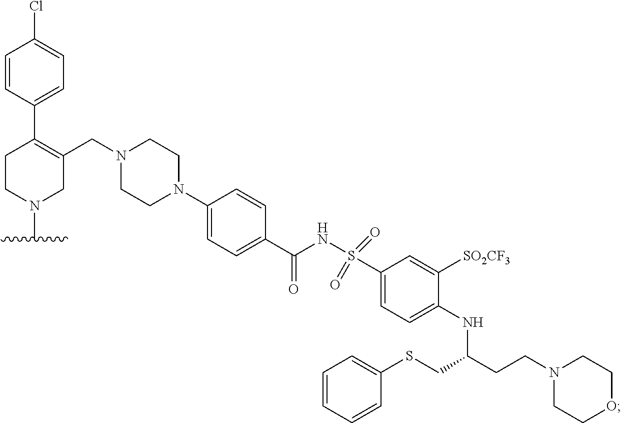
L₂ is 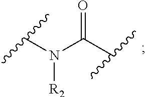
L₁ is independently 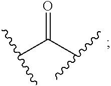 [SMILES: *C(*)=O]
and R is

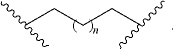

32. The compound of claim 1, or a pharmaceutically acceptable salt thereof, wherein Y is 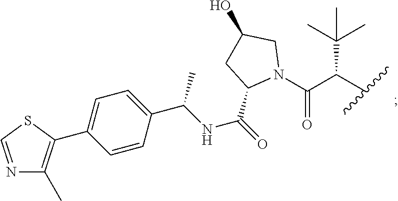 [SMILES: *[C@H](C(=O)N1C[C@H](O)C[C@H]1C(=O)N[C@@H](C)c1ccc(-c2scnc2C)cc1)C(C)(C)C]
Y₂ is 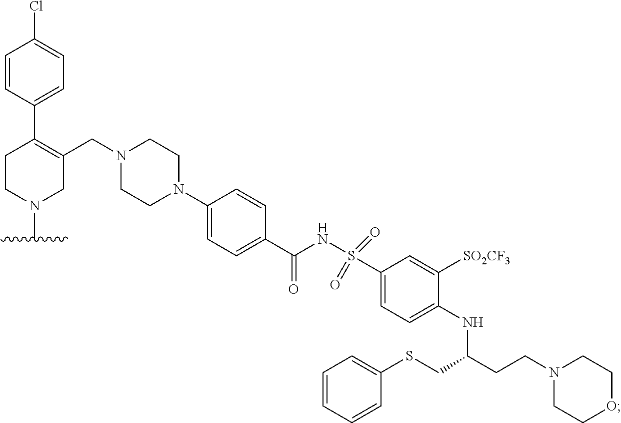
L₂ is 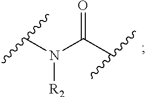
L₁ is independently 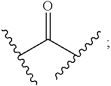 [SMILES: *C(*)=O]
R is 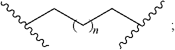
and n is 3-8, inclusive.

33. The compound of any one of claims 1-32, or a pharmaceutically acceptable salt thereof, wherein the compound is: 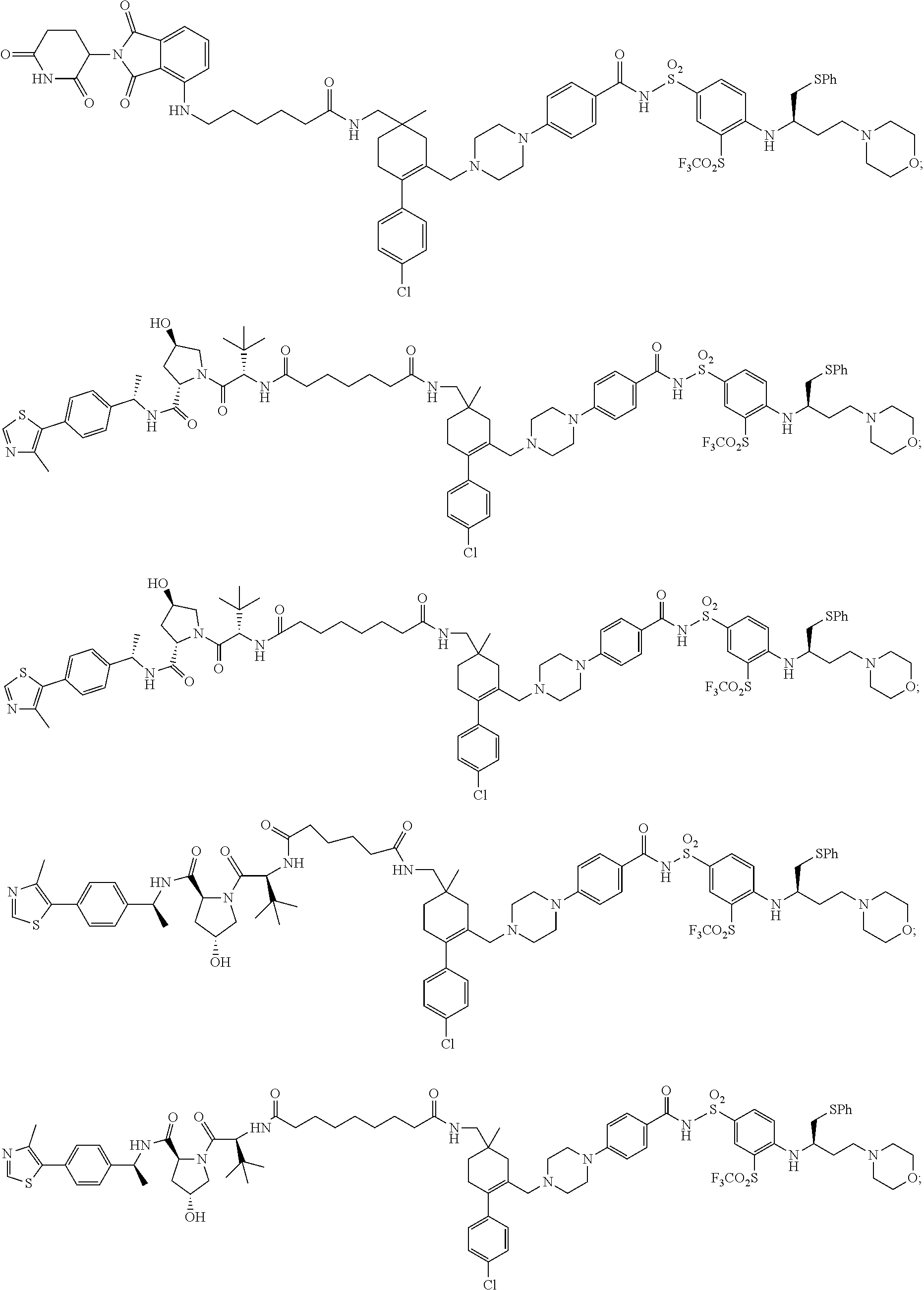 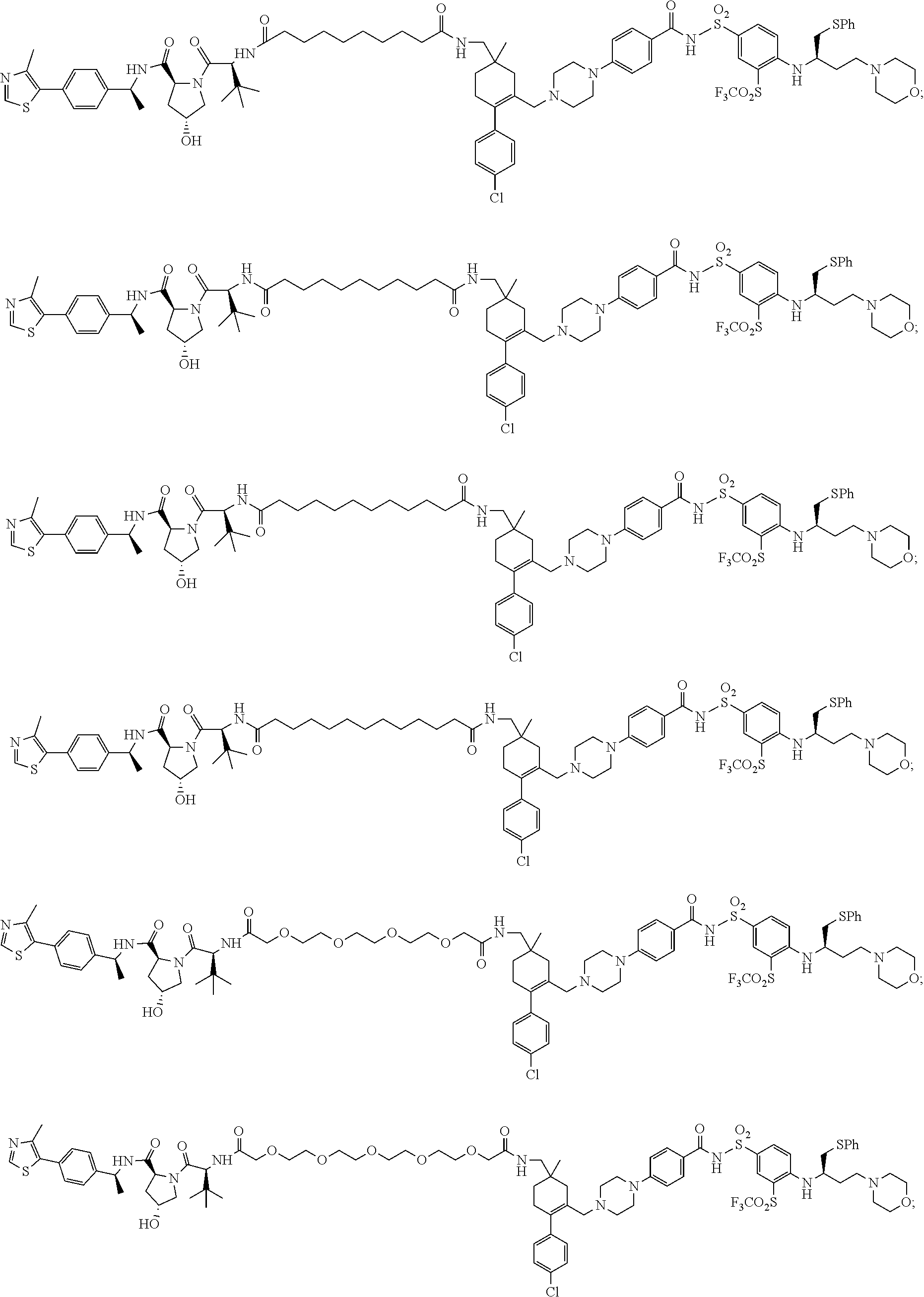 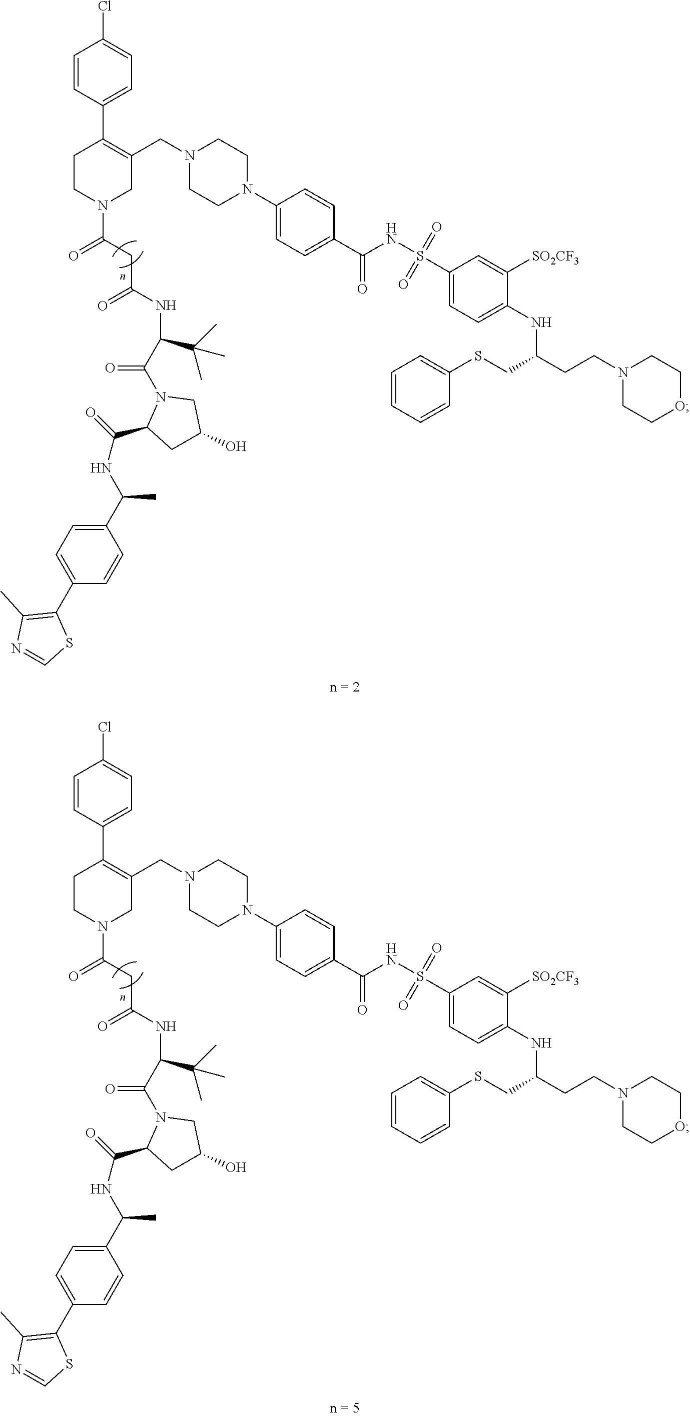 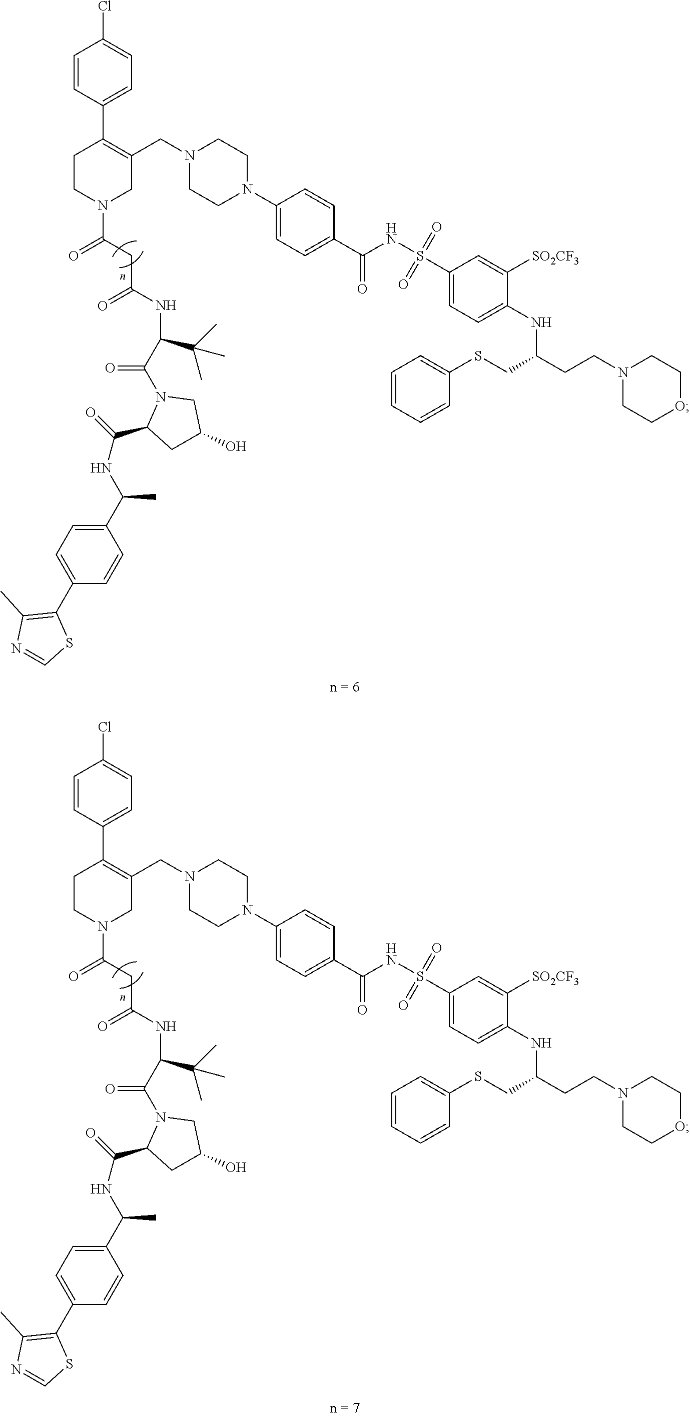 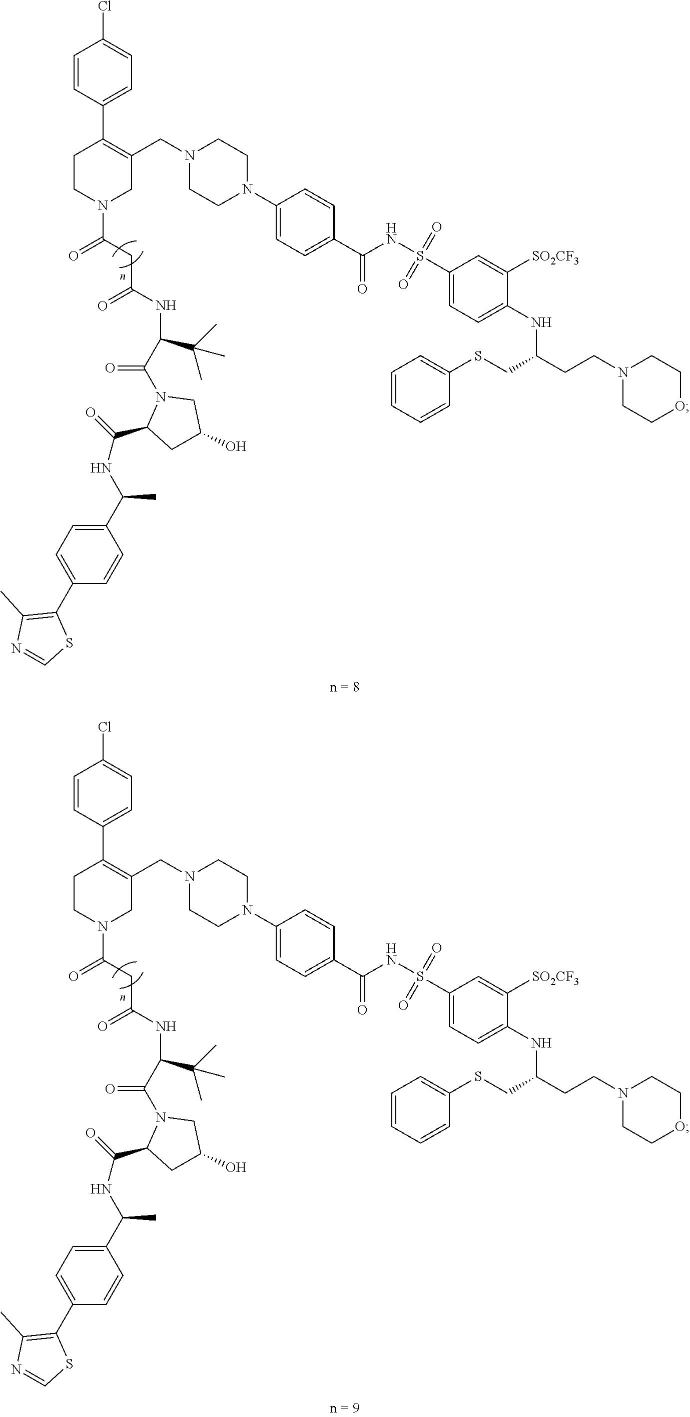 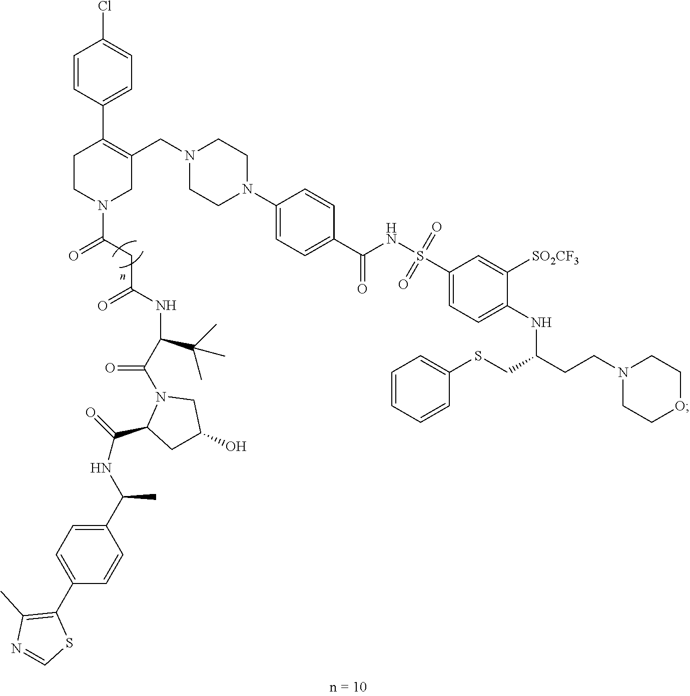 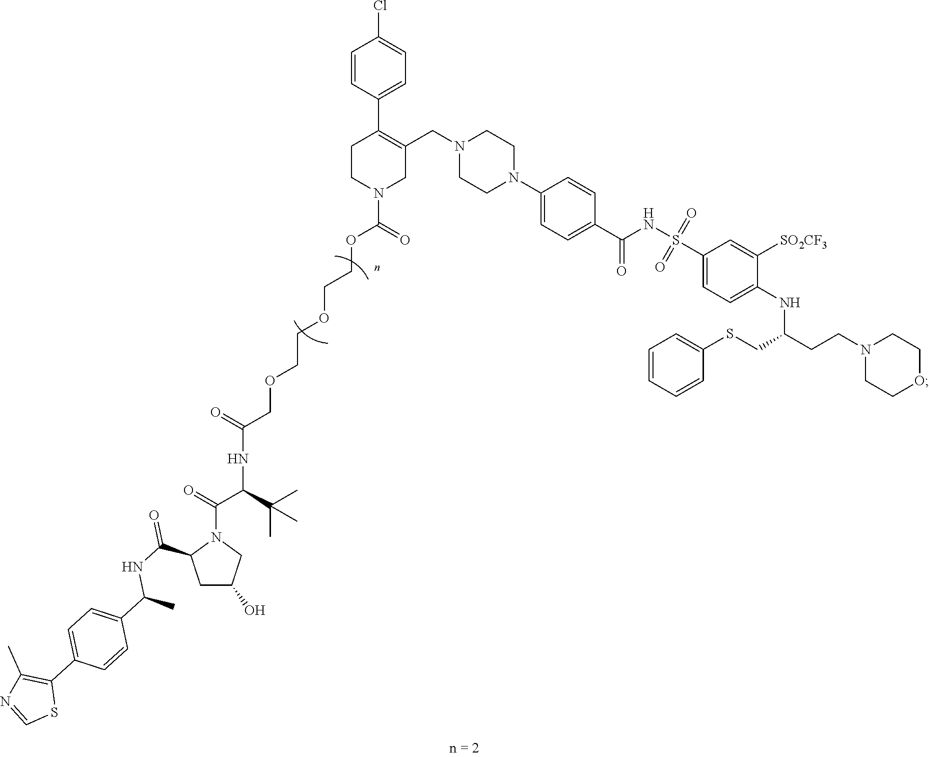 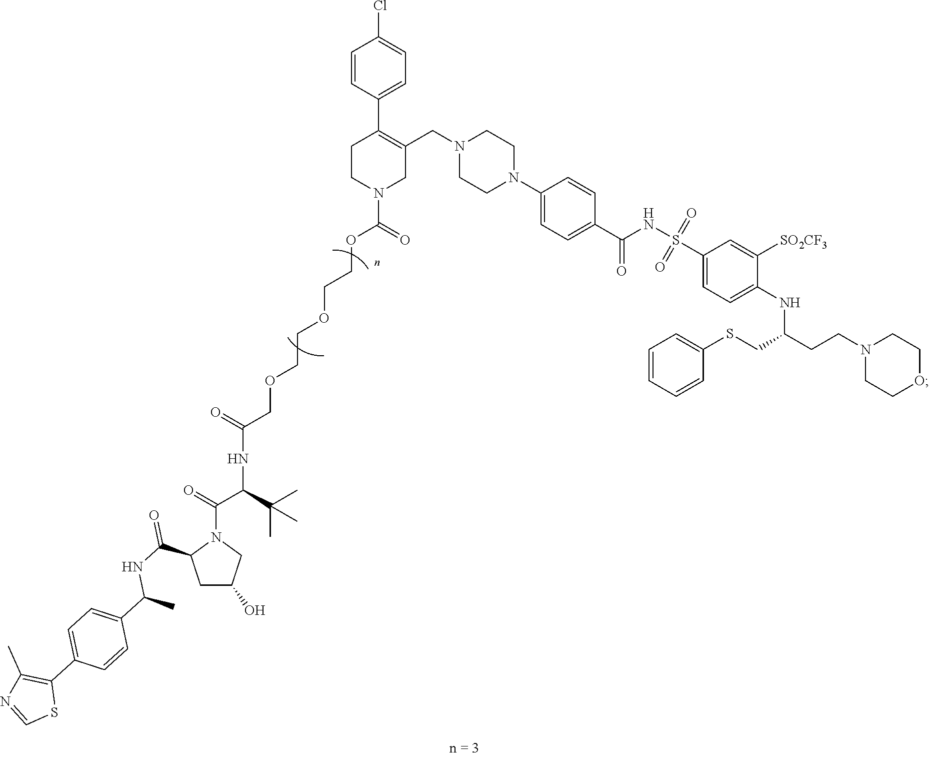
or a pharmaceutically acceptable salt thereof.

34. A pharmaceutical composition comprising a compound of any one of claims 1-33, or a pharmaceutically acceptable salt thereof, and a pharmaceutically acceptable carrier.

35. The pharmaceutical composition of claim 34, further comprising an additional agent.

36. The pharmaceutical composition of claim 35, wherein the additional agent is an anti-cancer agent.

37. The pharmaceutical composition of claim 36, wherein the anti-cancer agent is an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

38. A method of degrading Bcl-2 proteins, the method comprising administering an effective amount of a compound of any one of claims 1-33, or a pharmaceutically acceptable salt thereof.

39. The method of claim 38, wherein the compound is administered in vitro.

40. The method of claim 38, wherein the compound is administered in vivo.

41. The method of claim 38, further comprising administering the compound to a subject.

42. A method of treating a disease or disorder in a subject in need thereof, the method comprising administering an effective amount of a compound of any one of claims 1-33, or a pharmaceutically acceptable salt thereof.

43. A method of treating a subject suffering from or susceptible to a disease or disorder, the method comprising administering an effective amount of a compound of any one of claims 1-33, or a pharmaceutically acceptable salt thereof.

44. The method of claim 42 or 43, wherein the disease is cancer.

45. The method of claim 44, wherein the cancer is a solid tumor.

46. The method of claim 44, wherein the cancer is chronic lymphocyctic leukemia.

47. The method of claim 42 or 43, wherein the subject is a mammal.

48. The method of claim 42 or 43, wherein the subject is a human.

49. A method of treating a Bcl-2-mediated cancer in a subject in need thereof, the method comprising administering an effective amount of a compound of any one of claims 1-33, or a pharmaceutically acceptable salt thereof, wherein the platelet toxicity of the compound is less than that of other Bcl-2 inhibitors.

50. A method of treating a subject suffering from or susceptible to a Bcl-2-mediated cancer, the method comprising administering an effective amount of a compound of any one of claims 1-33, or a pharmaceutically acceptable salt thereof, wherein the platelet toxicity of the compound is less than that of other Bcl-2 inhibitors.

51. The method of claim 49 or 50, wherein the Bcl-2-mediated cancer is chronic lymphocyctic leukemia.

52. The method of claim 49 or 50, wherein the other Bcl-2 inhibitor is venetoclax or ABT-263.

53. A method of treating a Bcl-2-mediated cancer in a subject in need thereof, the method comprising administering an effective amount of a compound of any one of claims 1-33, or a pharmaceutically acceptable salt thereof, wherein the ratio of human platelet toxicity (IC₅₀) to anticancer activity (IC₅₀) of the compound is greater than one.

54. A method of treating a subject suffering from or susceptible to a Bcl-2-mediated cancer, the method comprising administering an effective amount of a compound of any one of claims 1-33, or a pharmaceutically acceptable salt thereof, wherein the ratio of human platelet toxicity (IC₅₀) to anticancer activity (IC₅₀) of the compound is greater than one.

55. The method of claim 53 or 54, wherein the Bcl-2-mediated cancer is chronic lymphocyctic leukemia.

56. The method of claim 53 or 54, wherein the anticancer activity is measured in MOLT-4 cells.

57. The method of claim 53 or 54, wherein the ratio is greater than 2.5.

58. The method of claim 53 or 54, wherein the ratio is greater than 5.

59. The method of claim 53 or 54, wherein the ratio is greater than 10.

60. The method of claim 53 or 54, wherein the ratio is greater than 20.

61. The method of claim 53 or 54, wherein the ratio is greater than 40.

62. A compound of Formula (I), or a pharmaceutically acceptable salt, hydrate, solvate, or prodrug thereof:
Y-L₂-R-L₁-Y₂ Formula (I);
wherein L₁ is independently 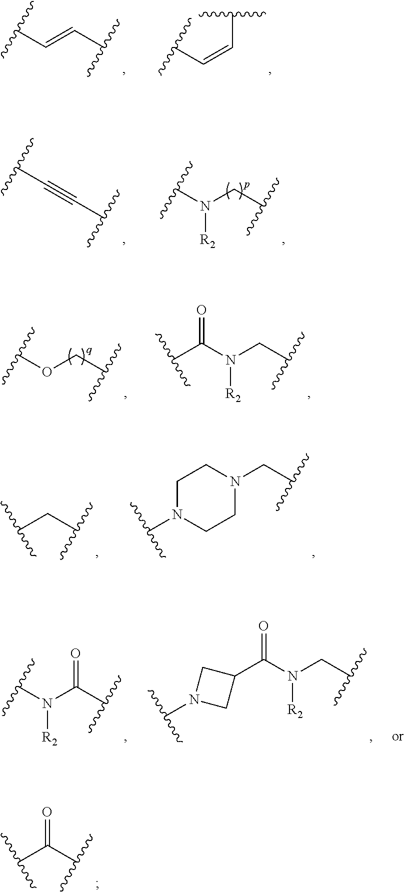
R is independently 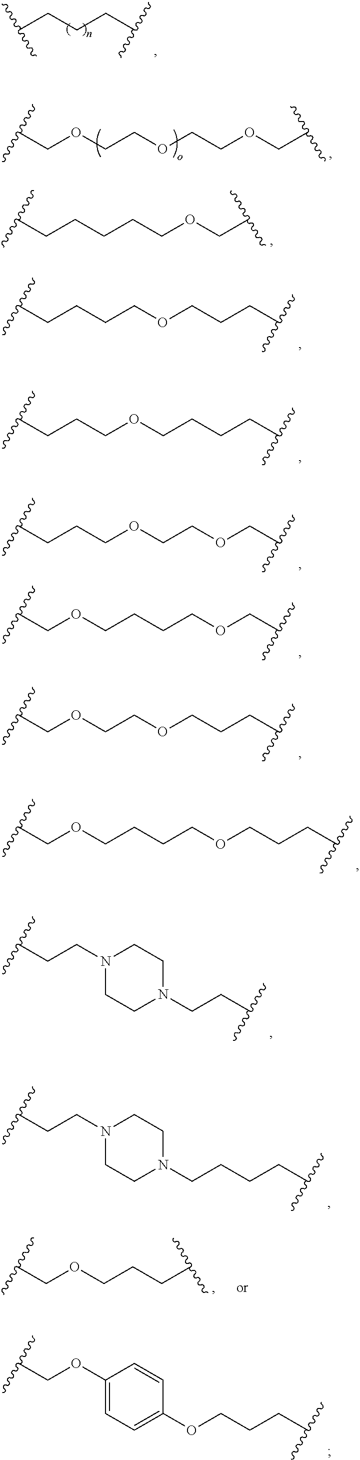
L₂ is independently 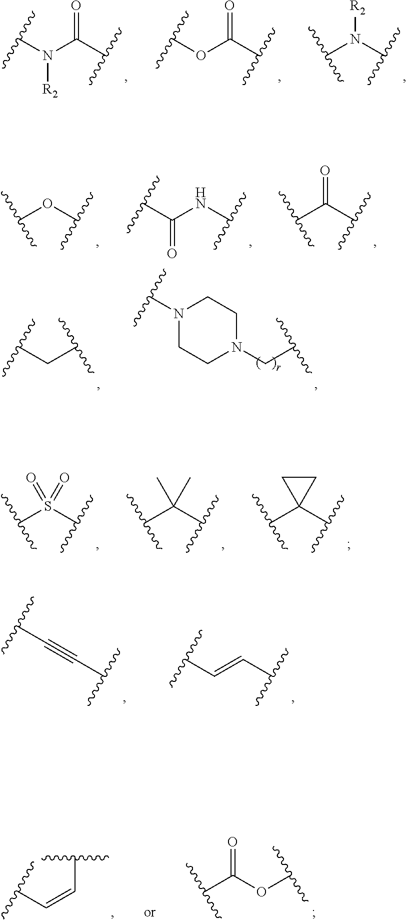
Y is independently 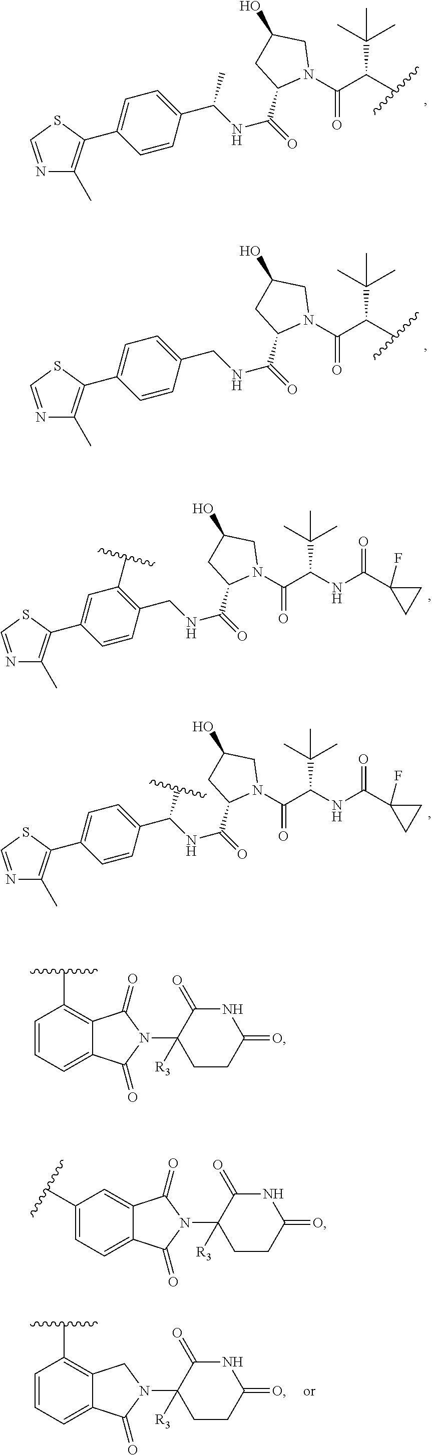 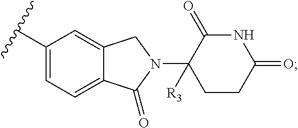
Y₂ is independently 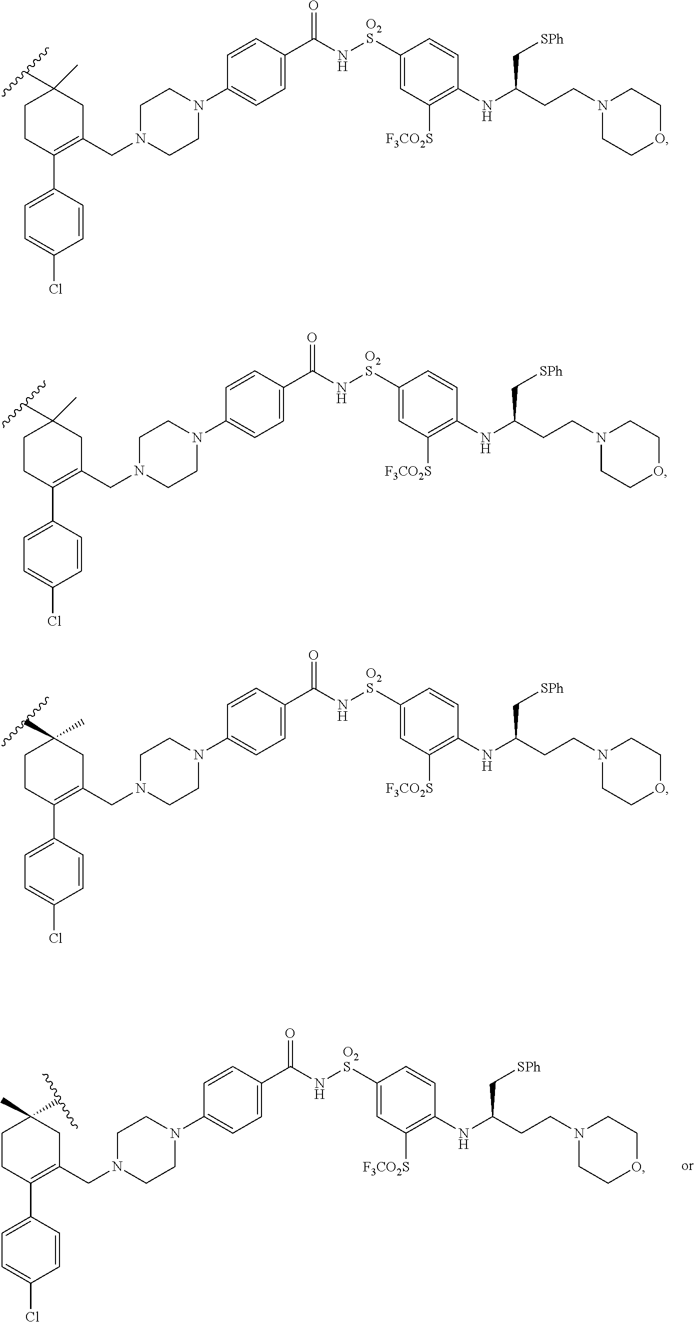 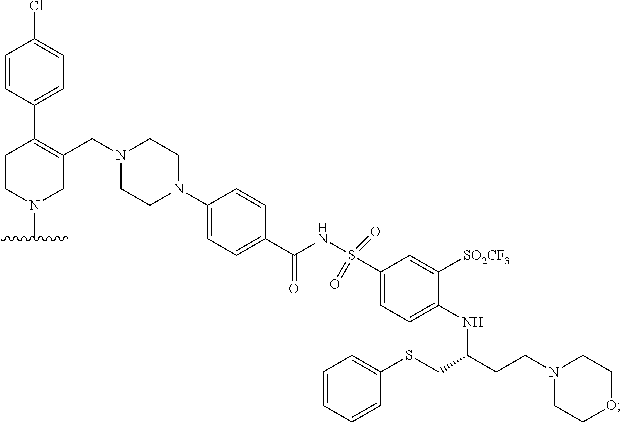
each R₂ is independently H, optionally substituted alkyl, or optionally substituted cycloalkyl;
each R₃ is independently H, D, CH₃, or F; and
each n, o, p, and q is independently 0-10, inclusive.

63. A compound of Table 3, or a pharmaceutically acceptable salt, hydrate, solvate, or prodrug thereof.

64. A pharmaceutical composition comprising a compound of claim 63, or a pharmaceutically acceptable salt thereof, and a pharmaceutically acceptable carrier.

65. The pharmaceutical composition of claim 64, further comprising an additional agent.

66. The pharmaceutical composition of claim 65, wherein the additional agent is an anti-cancer agent.

67. The pharmaceutical composition of claim 64, wherein the anti-cancer agent is an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof.

68. A method of degrading Bcl-2 proteins, the method comprising administering an effective amount of a compound of claim 63, or a pharmaceutically acceptable salt thereof.

69. The method of claim 68, wherein the compound is administered in vitro.

70. The method of claim 68, wherein the compound is administered in vivo.

71. The method of claim 68, further comprising administering the compound to a subject.

72. A method of treating a disease or disorder in a subject in need thereof, the method comprising administering an effective amount of a compound of claim 63, or a pharmaceutically acceptable salt thereof.

73. A method of treating a subject suffering from or susceptible to a disease or disorder, the method comprising administering an effective amount of a compound of claim 63, or a pharmaceutically acceptable salt thereof.

74. The method of claim 72 or 73, wherein the disease is cancer.

75. The method of claim 74, wherein the cancer is a solid tumor.

76. The method of claim 74, wherein the cancer is chronic lymphocyctic leukemia.

77. The method of claim 72 or 73, wherein the subject is a mammal.

78. The method of claim 72 or 73, wherein the subject is a human.

79. A method of treating a Bcl-2-mediated cancer in a subject in need thereof, the method comprising administering an effective amount of a compound of claim 63, or a pharmaceutically acceptable salt thereof, wherein the platelet toxicity of the compound is less than that of other Bcl-2 inhibitors.

80. A method of treating a subject suffering from or susceptible to a Bcl-2-mediated cancer, the method comprising administering an effective amount of a compound of claim 63, or a pharmaceutically acceptable salt thereof, wherein the platelet toxicity of the compound is less than that of other Bcl-2 inhibitors.

81. The method of claim 79 or 80, wherein the Bcl-2-mediated cancer is chronic lymphocyctic leukemia.

82. The compound of claim 1, or a pharmaceutically acceptable salt thereof, wherein Y is 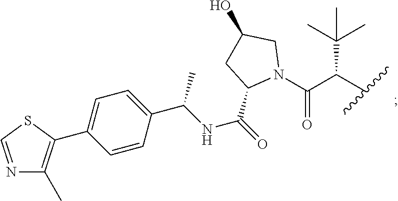 [SMILES: *[C@H](C(=O)N1C[C@H](O)C[C@H]1C(=O)N[C@@H](C)c1ccc(-c2scnc2C)cc1)C(C)(C)C]
Y₂ is 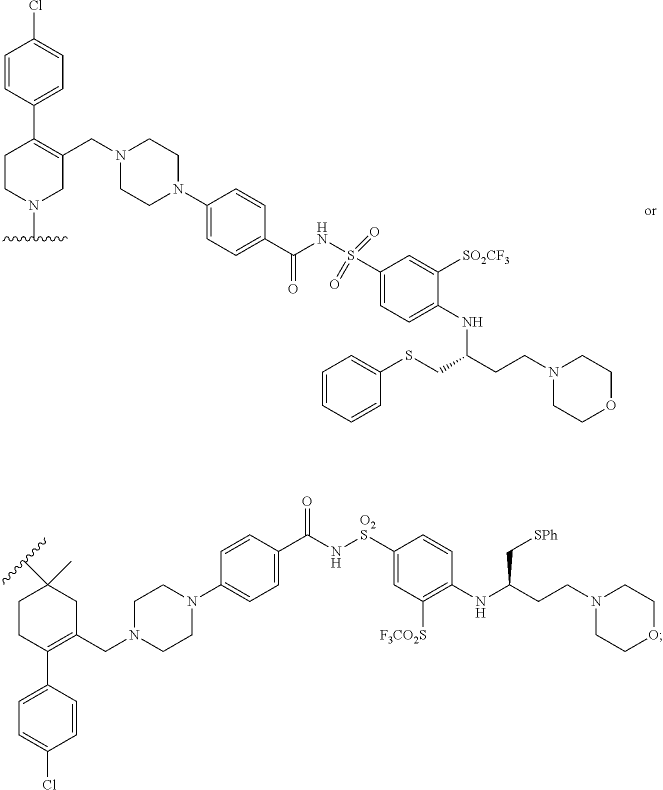
and
R is independently

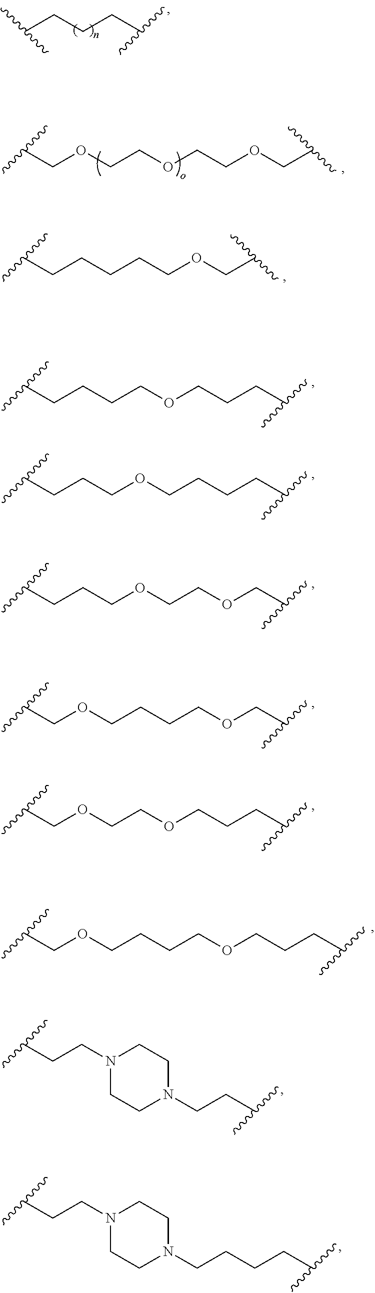

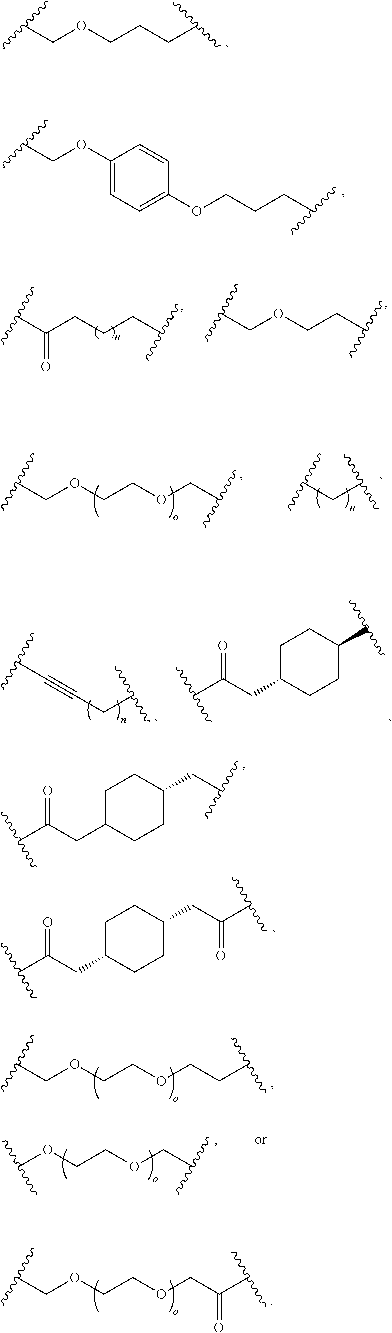

83. The compound of claim 1, or a pharmaceutically acceptable salt thereof, wherein Y is 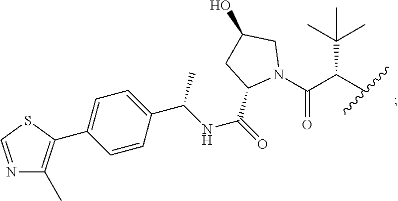 [SMILES: *[C@H](C(=O)N1C[C@H](O)C[C@H]1C(=O)N[C@@H](C)c1ccc(-c2scnc2C)cc1)C(C)(C)C]
Y₂ is 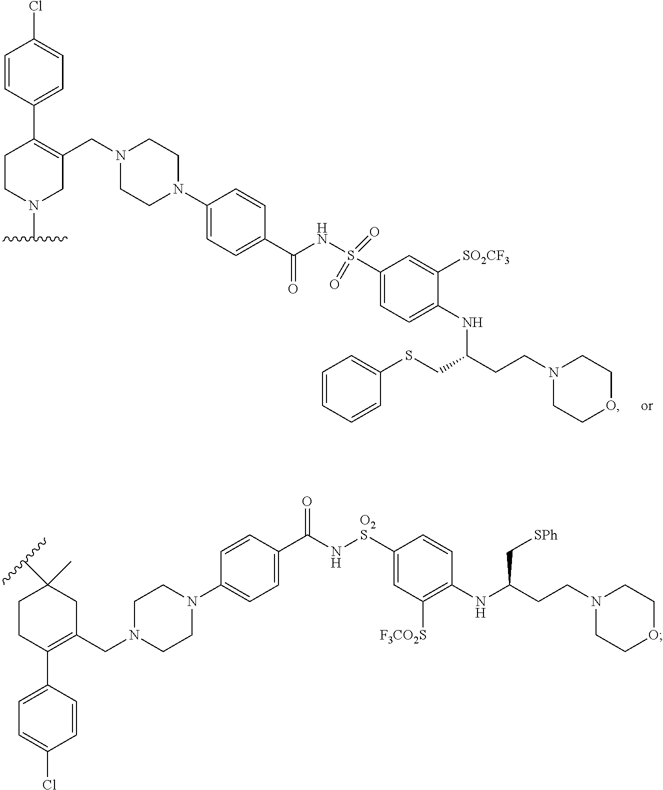
and
L₁ is independently 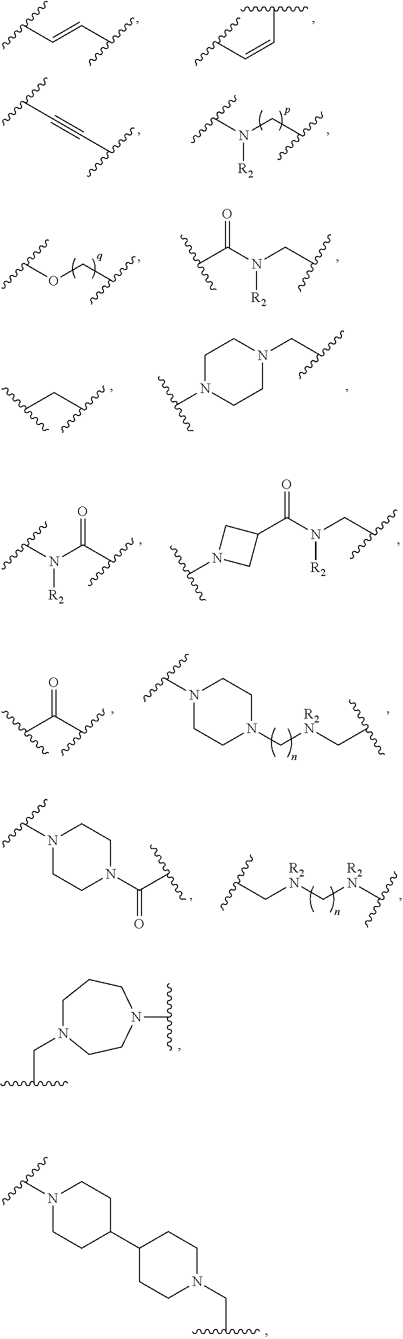 [chemical structure]
